(19) United States
(12) Patent Application Publication
(10) Pub. No.: US 2025/0195614 A1
(43) Pub. Date: Jun. 19, 2025

(54) METHOD OF TREATING PANCREATIC CANCER

(71) Applicant: IMMUNITYBIO, INC., San Diego, CA (US)

(72) Inventors: Hing C. WONG, Miramar, FL (US); Peter RHODE, Miramar, FL (US)

(21) Appl. No.: 18/843,074

(22) PCT Filed: Mar. 2, 2023
(86) PCT No.: PCT/US2023/063618

§ 371 Date: Aug. 30, 2024

Related U.S. Application Data
(60) Provisional application No. 63/315,820, filed on Mar. 2, 2022.
(60) Provisional application No. 63/315,840, filed on Mar. 2, 2022.

(51) Int. Cl. A61K 38/17; A61K 38/20; A61K 38/36; A61P 35/00

(52) U.S. Cl. CPC A61K 38/179; A61K 38/1793; A61K 38/2086; A61K 38/36; A61P 35/00

(57) ABSTRACT

Provided herein are methods of treating unresectable advanced/metastatic pancreatic cancer in a subject.

### CROSS-REFERENCE TO RELATED APPLICATIONS

This application claims priority to U.S. Provisional Patent Application Ser. No. 63/315,820, filed Mar. 2, 2022, and U.S. Provisional Patent Application Ser. No. 63/315,840, filed Mar. 2, 2022; the entire contents of which are herein incorporated by reference.

### SEQUENCE LISTING

This application contains a Sequence Listing that has been submitted electronically as an XML file named 47039-0030WO1.xml. The XML file, created on Feb. 23, 2023, is 96,770 bytes in size. The material in the XML file is hereby incorporated by reference in its entirety.

### TECHNICAL FIELD

The present disclosure relates to the field of biotechnology, and more specifically, to methods for treating pancreatic cancer in a subject using a multi-chain chimeric polypeptide.

### BACKGROUND

Tissue factor (TF), a 263 amino acid integral membrane glycoprotein with a molecular weight of ˜46 kDa and the trigger protein of the extrinsic blood coagulation pathway, is the primary initiator of coagulation in vivo. Tissue factor, normally not in contact with circulating blood, initiates the coagulation cascade upon exposure to the circulating coagulation serine protease factors. Vascular damage exposes sub-endothelial cells expressing tissue factor, resulting in the formation of a calcium-dependent, high-affinity complex with pre-existing plasma factor VIIa (FVIIa). Binding of the serine protease FVIIa to tissue factor promotes rapid cleavage of FX to FXa and FIX to FIXa. The proteolytic activity of the resulting FXa and an active membrane surface then inefficiently converts a small amount of prothrombin to thrombin. The thrombin generated by FXa initiates platelet activation and activates minute amounts of the pro-cofactors factor V (FV) and factor VIII (FVIII) to become active cofactors, factor Va (FVa) and factor VIIIa (FVIIIa). FIXa complexes with FVIIIa on the platelet surface forming the intrinsic tenase complex, which results in rapid generation of FXa. FXa complexes with FVa to form the pro-thrombinase complex on the activated platelet surface which results in rapid cleavage of prothrombin to thrombin.

In addition to the tissue factor-FVIIa complex, a recent study showed that the tissue factor-FVIIa-FXa complex can activate FVIII, which would provide additional levels of FVIIIa during the initiation phase. The extrinsic pathway is paramount in initiating coagulation via the activation of limited amounts of thrombin, whereas the intrinsic pathway maintains coagulation by dramatic amplification of the initial signal.

Much of the tissue factor expressed on a cell surface is “encrypted,” which must be “decrypted” for full participation in coagulation. The mechanism of “decryption” of cell-surface tissue factor is still unclear at this time, however, exposure of anionic phospholipids plays a major role in this process. Healthy cells actively sequester anionic phospholipids such as phosphatidyl serine (PS) to the inner leaflet of the plasma membrane. Following cellular damage, activation, or increased levels of cytosolic Ca²⁺, this bilayer asymmetry is lost, resulting in increased PS exposure on the outer leaflet, which increases the specific activity of cell-surface tissue factor-FVIIa complexes. PS exposure is known to decrease the apparent Km for activation of FIX and FX by tissue factor-FVIIa complexes, but additional mechanisms could include conformational rearrangement of tissue factor or tissue factor-FVIIa and subsequent exposure of substrate binding sites.

More than 62,000 Americans are expected to be diagnosed with pancreatic cancer in 2022 (see, Pancreatic Cancer Action Network website). Effective treatments for pancreatic cancer are desired.

### SUMMARY

The present invention is based on the discovery that a multi-chain chimeric polypeptide that includes (a) a first chimeric polypeptide including: (i) a first target-binding domain; (ii) a soluble tissue factor domain; and (iii) a first domain of a pair of affinity domains; and (b) a second chimeric polypeptide including: (i) a second domain of a pair of affinity domains; and (ii) a second target-binding domain, where the first chimeric polypeptide and the second chimeric polypeptide associate through the binding of the first domain and the second domain of the pair of affinity domains, and the first target-binding domain binds specifically to a ligand of TGF-β receptor II (TGF-βRII) and the second target-binding domain binds specifically to a ligand of TGF-βRII can provide treatment for pancreatic cancer in a subject.

Provided herein are methods of treating unresectable advanced/metastatic pancreatic cancer in a subject that include administering to the subject a therapeutically effective amount of a multi-chain chimeric polypeptide, wherein the multi-chain chimeric polypeptide comprises: (a) a first chimeric polypeptide comprising: (i) a first target-binding domain; (ii) a soluble tissue factor domain; and (iii) a first domain of a pair of affinity domains; (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII.

Also provided herein are methods of improving the objective response rate in subjects having unresectable advanced/metastatic pancreatic cancer that include administering to the subjects a therapeutically effective amount of a multi-chain chimeric polypeptide, wherein the multi-chain chimeric polypeptide comprises: (a) a first chimeric polypeptide comprising: (i) a first target-binding domain; (ii) a soluble tissue factor domain; and (iii) a first domain of a pair of affinity domains; (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII.

Also provided herein are methods of increasing progression-free survival or progression-free survival rate in a subject or population of subjects having unresectable advanced/metastatic pancreatic cancer that include administering to the subject(s) a therapeutically effective amount of a multi-chain chimeric polypeptide, wherein the multi-chain chimeric polypeptide comprises: (a) a first chimeric polypeptide comprising: (i) a first target-binding domain; (ii) a soluble tissue factor domain; and (iii) a first domain of a pair of affinity domains; (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII.

Also provided herein are methods of increasing time to progression in a subject or population of subjects having unresectable advanced/metastatic pancreatic cancer that include administering to the subject(s) a therapeutically effective amount of a multi-chain chimeric polypeptide, wherein the multi-chain chimeric polypeptide comprises: (a) a first chimeric polypeptide comprising: (i) a first target-binding domain; (ii) a soluble tissue factor domain; and (iii) a first domain of a pair of affinity domains; (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-receptor II (TGF-βRII) and the second target-binding domain binds specifically to a ligand of TGF-βRII.

Also provided herein are methods of increasing duration of response in a subject or population of subjects having unresectable advanced/metastatic pancreatic cancer that include administering to the subject(s) a therapeutically effective amount of a multi-chain chimeric polypeptide, wherein the multi-chain chimeric polypeptide comprises: (a) a first chimeric polypeptide comprising: (i) a first target-binding domain; (ii) a soluble tissue factor domain; and (iii) a first domain of a pair of affinity domains; (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII.

Also provided herein are methods of increasing overall survival in a population of subjects having unresectable advanced/metastatic pancreatic cancer that include administering to each subject of the population a therapeutically effective amount of a multi-chain chimeric polypeptide, wherein the multi-chain chimeric polypeptide comprises: (a) a first chimeric polypeptide comprising: (i) a first target-binding domain; (ii) a soluble tissue factor domain; and (iii) a first domain of a pair of affinity domains; (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII.

In some embodiments of any of the methods described herein, the subject(s) has/have an age of 18 years or more. In some embodiments of any of the methods described herein, the subject(s) has/have received previous treatment with standard first-line systemic therapy for pancreatic cancer, and the subject's/subjects' pancreatic cancer had progressed on and/or was intolerant to the previous treatment. In some embodiments of any of the methods described herein, the subject(s) has/have received previous treatment with standard first-line systemic therapy for pancreatic cancer, and the subject(s) was/were intolerant to the first-line systemic therapy. In some embodiments of any of the methods described herein, the standard first-line systemic therapy comprises one or more of: FOLFIRINOX, modified FOLFINIROX, gemcitabine, albumin-bound paclitaxel, cisplatin, erlotinib, capecitabine, docetaxel, fluoropyrimidine, and oxaliplatin. In some embodiments of any of the methods described herein, the first-line systemic therapy comprises one of: (i) FOLFIRINOX; (ii) modified FOLFIRINOX; (iii) gemcitabine and albumin-bound paclitaxel; (iv) gemcitabine and erlotinib; (v) gemcitabine; (vi) gemcitabine and capecitabine; (vii) gemcitabine, docetaxel, and capecitabine; and (viii) fluoropyrimidine and oxaliplatin. In some embodiments of any of the methods described herein, the subject(s) has/have previously been identified as having a BRCA1, BRCA2, or PALB2 mutation, and the first-line systemic therapy comprises one of: (i) FOLFIRINOX; (ii) modified FOLFIRINOX; and (iii) gemcitabine and cisplatin.

In some embodiments of any of the methods described herein, the subject(s) has/have received previous treatment with second- or later-line systemic therapy for pancreatic cancer, and the subject's/subjects' pancreatic cancer had progressed on and/or was intolerant to the previous treatment. In some embodiments of any of the methods described herein, the second- or later-line systemic therapy comprises one or more of: a different first-line systemic therapy, 5-fluorouracil, leucovorin, liposomal irinotecan, irinotecan, FOLFIRINOX, modified FOLFIRINOX, oxaliplatin, FOLFOX, capecitabine, gemcitabine, albumin-bound paclitaxel, cisplatin, erlotinib, pembrolizumab, larotrectinib, and entrectinib. In some embodiments of any of the methods described herein, the second- or later-line systemic therapy is a different first-line systemic therapy. In some embodiments of any of the methods described herein, the second- or later-line systemic therapy comprises one of: (i) 5-fluorouracil, leucovorin, and liposomal irinotecan; (ii) 5-fluorouracil, leucovorin, and irinotecan (FOLFIRI); (iii) FOLFIRINOX or modified FOLFIRINOX; (iv) oxaliplatin, 5-fluorouracil, and leucovorin (OFF), (v) FOLFOX; (vi) capecitabine and oxaliplatin; (vii) capecitabine; and (viii) continuous infusion 5-fluorouracil. In some embodiments of any of the methods described herein, the subject(s) was/were previously treated with fluoropyrimidine-based therapy and the second- or later-line systemic therapy comprises one of: (i) gemcitabine; (ii) gemcitabine and albumin-bound paclitaxel; and (iii) gemcitabine with erlotinib. In some embodiments of any of the methods described herein, the subject(s) was/were previously treated with fluoropyrimidine-based therapy and was/were previously identified as having a BRCA1, BRCA2, or PALB2 mutation, and the second- or later-line systemic therapy comprises gemcitabine and cisplatin. In some embodiments of any of the methods described herein, the subject(s) was/were previously treated with fluoropyrimidine-based therapy and has/have not received prior treatment with irinotecan, and the second- or later-line systemic therapy comprises 5-fluorouracil, leucovorin, and liposomal irinotecan. In some embodiments of any of the methods described herein, the subject(s) was/were previously identified as having an MSI-H or dMMR tumor, and the second- or later-line systemic therapy comprises pembrolizumab. In some embodiments of any of the methods described herein, the subject(s) was/were previously identified as having a NTRK gene fusion, and the second- or later-line systemic therapy comprises larotrectinib or entrectinib.

In some embodiments of any of the methods described herein, the subject(s) has/have distant metastatic disease. In some embodiments of any of the methods described herein, the subject(s) has/have adequate cardiac, pulmonary, liver, and kidney function. In some embodiments of any of the methods described herein, the subject(s) has/have an Eastern Cooperative Oncology Group (ECOG) performance status of 0, 1, or 2. In some embodiments of any of the methods described herein, the subject(s) has/have a life expectancy, prior to the administering step, of at least 12 weeks.

In some embodiments of any of the methods described herein, subject(s), prior to the administering step, has/have been determined to have measurable disease as assessed by imaging studies. In some embodiments of any of the methods described herein, the subject(s) has/have received prior radiation therapy at least four weeks before the administering step. In some embodiments of any of the methods described herein, any acute effects of any prior therapy in the subject(s) has/have reduced to baseline or a grade less than or equal to 1 NCI CTCAE v5.0, before the administering step.

In some embodiments of any of the methods described herein, the subject(s) has/have: an absolute neutrophil count of greater than or equal to 1,500/microliter; a platelet count of greater than or equal to 100,000/microliter; a hemoglobin level of greater than or equal to 9 g/dL; a glomerular filtration rate (GFR) of greater than 40 mL/min or serum creatinine level of less than or equal to 1.5×Upper Limit of Normal (ULN); a total bilirubin level of less than or equal to 2.0×ULN or less than or equal to 3.0×ULN for subjects having Gilbert's syndrome; and aspartate aminotransferase (AST), alanine aminotransferase (ALT), and alkaline phosphatase (ALP) levels of less than or equal to 2.5×ULN or less than or equal to 5.0×ULN if liver metastasis is present. In some embodiments of any of the methods described herein, the subject(s) has/have a level of Pulmonary Function Test (PFT) greater than 50% Forced Expiratory Volume (FEV1) if symptomatic or prior known impairment.

In some embodiments of any of the methods described herein, the subject(s) is/are female, and the female(s) has/have had a negative pregnancy test within 14 days prior to the administering step. In some embodiments of any of the methods described herein, the female(s) has/have received birth control at least 14 days prior, and during, the administering step, or is surgically sterilized.

In some embodiments of any of the methods described herein, the subject(s) is/are male, and the subject(s) uses/use barrier method birth control during the administering step, and at least 28 days after the administering step.

In some embodiments of any of the methods described herein, the subject(s) does/do not have a history of clinically significant vascular disease. In some embodiments of any of the methods described herein, the subject(s) does/do not have a Corrected QT interval (QTc) of greater than or equal to 470 milliseconds by Fridericia's correction. In some embodiments of any of the methods described herein, the subject(s) does/do not have an untreated CNS metastasis. In some embodiments of any of the methods described herein, the subject(s) has/have received prior treatment for CNS metastasis and the subject(s) is/are neurologically stable for at least two weeks prior to the administering step. In some embodiments of any of the methods described herein, the subject(s) is/are not receiving, during the administering step, a corticosteroid. In some embodiments of any of the methods described herein, the subject(s) is/are receiving a stable or decreasing dose of a corticosteroid of less than or equal to 10 mg daily, during the administering step.

In some embodiments of any of the methods described herein, the subject(s) has/have not received surgery, radiotherapy, chemotherapy, other immunotherapy, or investigational therapy within 14 days prior to the administering step. In some embodiments of any of the methods described herein, the subject(s) does/do not have any other prior malignancy except for adequately-treated basal cell or squamous cell skin cancer, in situ cervical cancer, adequately-treated stage I or II cancer from which the subject(s) is/are currently in complete remission, or any other cancer from which the subject(s) has/have been disease-free for 3 years after surgical treatment.

In some embodiments of any of the methods described herein, the subject(s) does/do not have known hypersensitivity or a history of allergic reactions attributed to compounds of similar chemical or biological composition to the multi-chain chimeric polypeptide. In some embodiments of any of the methods described herein, the subject(s) has/have not received prior treatment with a TGF-beta antagonist or IL-15 or analog thereof.

In some embodiments of any of the methods described herein, the subject(s) is/are not receiving concurrent herbal or unconventional therapy. In some embodiments of any of the methods described herein, the subject(s) does/do not have an autoimmune disease requiring active treatment. In some embodiments of any of the methods described herein, the subject(s) does/do not have a condition requiring systemic treatment with a corticosteroid or an immunosuppressive treatment within 14 days of the administering step. In some embodiments of any of the methods described herein, the subject(s) does/do not have active autoimmune disease, and has received inhaled or topical steroids or adrenal replacement steroid doses of equal to or less than 10 mg daily prednisone equivalent.

In some embodiments of any of the methods described herein, the subject(s) does/do not have an active systemic infection requiring parenteral antibiotic therapy. In some embodiments of any of the methods described herein, the subject(s) has/have not previously received an organ allograft or allogeneic transplantation. In some embodiments of any of the methods described herein, the subject(s) has/have not been identified or diagnosed as being HIV-positive or having AIDS. In some embodiments of any of the methods described herein, the subject(s) is/are a female and the female(s) is/are not pregnant or nursing.

In some embodiments of any of the methods described herein, the subject(s) does/do not have any ongoing toxicity from a prior treatment. In some embodiments of any of the methods described herein, the ongoing toxicity is greater than grade 1 using NCI CTCAE v5.0 or greater than baseline. In some embodiments of any of the methods described herein, the ongoing toxicity excludes peripheral neuropathy, alopecia, and fatigue. In some embodiments of any of the methods described herein, the subject(s) does/do not have psychiatric illness.

In some embodiments of any of the methods described herein, the first target-binding domain and the soluble tissue factor domain directly abut each other in the first chimeric polypeptide. In some embodiments of any of the methods described herein, the first chimeric polypeptide further comprises a linker sequence between the first target-binding domain and the soluble tissue factor domain in the first chimeric polypeptide. In some embodiments of any of the methods described herein, the soluble tissue factor domain and the first domain of the pair of affinity domains directly abut each other in the first chimeric polypeptide. In some embodiments of any of the methods described herein, the first chimeric polypeptide further comprises a linker sequence between the soluble tissue factor domain and the first domain of the pair of affinity domains in the first chimeric polypeptide.

In some embodiments of any of the methods described herein, the second domain of the pair of affinity domains and the second target-binding domain directly abut each other in the second chimeric polypeptide. In some embodiments of any of the methods described herein, the second chimeric polypeptide further comprises a linker sequence between the second domain of the pair of affinity domains and the second target-binding domain in the second chimeric polypeptide.

In some embodiments of any of the methods described herein, one or both of the first target-binding domain and the second target-binding domain is an antigen-binding domain. In some embodiments of any of the methods described herein, one or both of the first target-binding domain and the second target-binding domain is a soluble interleukin or cytokine receptor. In some embodiments of any of the methods described herein, the first chimeric polypeptide further comprises one or more additional target-binding domain(s). In some embodiments of any of the methods described herein, the second chimeric polypeptide further comprises one or more additional target-binding domain(s).

In some embodiments of any of the methods described herein, the soluble tissue factor domain is a soluble human tissue factor domain. In some embodiments of any of the methods described herein, the soluble human tissue factor domain comprises a sequence that is at least 80% identical to SEQ ID NO: 1. In some embodiments of any of the methods described herein, the pair of affinity domains is a sushi domain from an alpha chain of human IL-15 receptor (IL-15Rα) and a soluble IL-15.

In some embodiments of any of the methods described herein, the first target-binding domain comprises a soluble TGF-βRII. In some embodiments of any of the methods described herein, the first target-binding domain comprises a first sequence that is at least 80% identical to SEQ ID NO: 66 and a second sequence that is at least 80% identical to SEQ ID NO: 66, wherein the first and second sequence are separated by a linker. In some embodiments of any of the methods described herein, the first target-binding domain comprises a first sequence that is at least 90% identical to SEQ ID NO: 66 and a second sequence that is at least 90% identical to SEQ ID NO: 66. In some embodiments of any of the methods described herein, the first target-binding domain comprises a first sequence of SEQ ID NO: 66 and a second sequence of SEQ ID NO: 66.

In some embodiments of any of the methods described herein, the linker comprises a sequence of SEQ ID NO: 7.

In some embodiments of any of the methods described herein, the first target-binding domain comprises a sequence that is at least 80% identical to SEQ ID NO: 69. In some embodiments of any of the methods described herein, the first target-binding domain comprises a sequence that is at least 90% identical to SEQ ID NO: 69. In some embodiments of any of the methods described herein, the first target-binding domain comprises a sequence of SEQ ID NO: 69.

In some embodiments of any of the methods described herein, the first chimeric polypeptide comprises a sequence that is at least 80% identical to SEQ ID NO: 70. In some embodiments of any of the methods described herein, the first chimeric polypeptide comprises a sequence that is at least 90% identical to SEQ ID NO: 70. In some embodiments of any of the methods described herein, the first chimeric polypeptide comprises a sequence of SEQ ID NO: 70. In some embodiments of any of the methods described herein, the first chimeric polypeptide comprises a sequence of SEQ ID NO: 72.

In some embodiments of any of the methods described herein, the second target-binding domain comprises a soluble TGF-βRII. In some embodiments of any of the methods described herein, the second target-binding domain comprises a first sequence that is at least 80% identical to SEQ ID NO: 66 and a second sequence that is at least 80% identical to SEQ ID NO: 66, wherein the first and second sequence are separated by a linker. In some embodiments of any of the methods described herein, the second target-binding domain comprises a first sequence that is at least 90% identical to SEQ ID NO: 66 and a second sequence that is at least 90% identical to SEQ ID NO: 66. In some embodiments of any of the methods described herein, the second target-binding domain comprises a first sequence of SEQ ID NO: 66 and a second sequence of SEQ ID NO: 66.

In some embodiments of any of the methods described herein, the linker comprises a sequence of SEQ ID NO: 7.

In some embodiments of any of the methods described herein, the second target-binding domain comprises a sequence that is at least 80% identical to SEQ ID NO: 69. In some embodiments of any of the methods described herein, the second target-binding domain comprises a sequence that is at least 90% identical to SEQ ID NO: 69. In some embodiments of any of the methods described herein, the second target-binding domain comprises a sequence of SEQ ID NO: 69.

In some embodiments of any of the methods described herein, the second chimeric polypeptide comprises a sequence that is at least 80% identical to SEQ ID NO: 74. In some embodiments of any of the methods described herein, the first chimeric polypeptide comprises a sequence that is at least 80% identical to SEQ ID NO: 70. In some embodiments of any of the methods described herein, the second chimeric polypeptide comprises a sequence that is at least 90% identical to SEQ ID NO: 74. In some embodiments of any of the methods described herein, the first chimeric polypeptide comprises a sequence that is at least 90% identical to SEQ ID NO: 70. In some embodiments of any of the methods described herein, the second chimeric polypeptide comprises a sequence of SEQ ID NO: 74. In some embodiments of any of the methods described herein, the first chimeric polypeptide comprises a sequence of SEQ ID NO: 70.

In some embodiments of any of the methods described herein, the multi-chain chimeric polypeptide is subcutaneously administered to the subject(s). In other embodiments of any of the methods described herein, the multi-chain chimeric polypeptide is administered to the subject(s) intravenously, intraperitoneally, intramuscularly, intratumorally, or subdermally. In some embodiments of any of the methods described herein, the subject(s) is/are administered a single dose of the multi-chain chimeric polypeptide. In some embodiments of any of the methods described herein, the single dose is 0.1 mg of the multi-chain chimeric polypeptide per kg of the subject's body weight (mg/kg). In some embodiments of any of the methods described herein, the single dose is 0.25 mg/kg. In some embodiments of any of the methods described herein, the single dose is 0.5 mg/kg. In some embodiments of any of the methods described herein, the single dose is 0.8 mg/kg. In some embodiments of any of the methods described herein, the single dose is 1.2 mg/kg.

In some embodiments of any of the methods described herein, the subject(s) is/are administered two or more doses of the multi-chain chimeric polypeptide over a treatment period. In some embodiments of any of the methods described herein, at least one of the two or more doses is 0.1 mg of the multi-chain chimeric polypeptide per kg of the subject's body weight (mg/kg). In some embodiments of any of the methods described herein, at least one of the two or more doses is 0.25 mg/kg. In some embodiments of any of the methods described herein, at least one of the two or more doses is 0.5 mg/kg. In some embodiments of any of the methods described herein, at least one of the two or more doses is 0.8 mg/kg. In some embodiments of any of the methods described herein, at least one of the two or more doses is 1.2 mg/kg. In some embodiments of any of the methods described herein, the treatment period is about 4 weeks.

As used herein, the term “chimeric” refers to a polypeptide that includes amino acid sequences (e.g., domains) originally derived from two different sources (e.g., two different naturally-occurring proteins, e.g., from the same or different species). For example, a chimeric polypeptide can include domains from at least two different naturally occurring human proteins. In some examples, a chimeric polypeptide can include a domain that is a synthetic sequence (e.g., an scFv) and a domain that is derived from a naturally-occurring protein (e.g., a naturally-occurring human protein). In some embodiments, a chimeric polypeptide can include at least two different domains that are synthetic sequences (e.g., two different scFvs).

An “antigen-binding domain” is one or more protein domain(s) (e.g., formed from amino acids from a single polypeptide or formed from amino acids from two or more polypeptides (e.g., the same or different polypeptides) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof. In some embodiments, an antigen-binding domain can include an alternative scaffold. Non-limiting examples of antigen-binding domains are described herein. Additional examples of antigen-binding domains are known in the art.

A “soluble tissue factor domain” refers to a polypeptide having at least 70% identity (e.g., at least 75% identity, at least 80% identity, at least 85% identity, at least 90% identity, at least 95% identity, at least 99% identity, or 100% identical) to a segment of a wildtype mammalian tissue factor protein (e.g., a wildtype human tissue factor protein) that lacks the transmembrane domain and the intracellular domain. Non-limiting examples of soluble tissue factor domains are described herein.

The term “soluble interleukin receptor” is used herein in the broadest sense to refer to a polypeptide that lacks a transmembrane domain (and optionally an intracellular domain) that is capable of binding one or more of its natural ligands (e.g., under physiological conditions, e.g., in phosphate buffered saline at room temperature). For example, a soluble interleukin receptor can include a sequence that is at least 70% identical (e.g., at least 75% identical, at least 80% identical, at least 85% identical, at least 90% identical, at least 95% identical, at least 99% identical, or 100% identical) to an extracellular domain of wildtype interleukin receptor and retains its ability to specifically bind to one or more of its natural ligands, but lacks its transmembrane domain (and optionally, further lacks its intracellular domain). Non-limiting examples of soluble interleukin receptors are described herein.

The term “soluble cytokine receptor” is used herein in the broadest sense to refer to a polypeptide that lacks a transmembrane domain (and optionally an intracellular domain) that is capable of binding one or more of its natural ligands (e.g., under physiological conditions, e.g., in phosphate buffered saline at room temperature). For example, a soluble cytokine receptor can include a sequence that is at least 70% identical (e.g., at least 75% identical, at least 80% identical, at least 85% identical, at least 90% identical, at least 95% identical, at least 99% identical, or 100% identical) to an extracellular domain of wildtype cytokine receptor and retains its ability to specifically bind to one or more of its natural ligands, but lacks its transmembrane domain (and optionally, further lacks its intracellular domain). Non-limiting examples of soluble cytokine receptors are described herein.

The term “antibody” is used herein in its broadest sense and includes certain types of immunoglobulin molecules that include one or more antigen-binding domains that specifically bind to an antigen or epitope. An antibody specifically includes, e.g., intact antibodies (e.g., intact immunoglobulins), antibody fragments, and multi-specific antibodies. One example of an antigen-binding domain is an antigen-binding domain formed by a VH-VL dimer. Additional examples of an antibody are described herein. Additional examples of an antibody are known in the art.

“Affinity” refers to the strength of the sum total of non-covalent interactions between an antigen-binding site and its binding partner (e.g., an antigen or epitope). Unless indicated otherwise, as used herein, “affinity” refers to intrinsic binding affinity, which reflects a 1:1 interaction between members of an antigen-binding domain and an antigen or epitope. The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein. Affinity can be determined, for example, using surface plasmon resonance (SPR) technology (e.g., BIACORE®) or biolayer interferometry (e.g., FORTEBIO®). Additional methods for determining the affinity for an antigen-binding domain and its corresponding antigen or epitope are known in the art.

A “multi-chain polypeptide” as used herein to refers to a polypeptide comprising two or more (e.g., three, four, five, six, seven, eight, nine, or ten) protein chains (e.g., at least a first chimeric polypeptide and a second polypeptide), where the two or more proteins chains associate through non-covalent bonds to form a quaternary structure.

The term “pair of affinity domains” is two different protein domain(s) that bind specifically to each other with a K_{D} of less than of less than 1×10⁻⁷ M (e.g., less than 1×10⁻⁸ M, less than 1×10⁻⁹ M, less than 1×10⁻¹⁰ M, or less than 1×10⁻¹¹ M). In some examples, a pair of affinity domains can be a pair of naturally-occurring proteins. In some embodiments, a pair of affinity domains can be a pair of synthetic proteins. Non-limiting examples of pairs of affinity domains are described herein.

The term “epitope” means a portion of an antigen that specifically binds to an antigen-binding domain. Epitopes can, e.g., consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. Methods for identifying an epitope to which an antigen-binding domain binds are known in the art.

The term “treatment” means to ameliorate at least one symptom of a disorder. In some examples, the disorder being treated is cancer and to ameliorate at least one symptom of cancer includes, e.g., reducing aberrant proliferation, gene expression, signaling, translation, and/or secretion of factors. In some embodiments, treatment of cancer can include, e.g., decreasing the rate of progression of cancer in the subject and/or the rate of development of metastasis in a subject (e.g., as compared to the rate of progression of cancer and/or the rate of development of metastasis in a similar subject not receiving treatment or receiving a different treatment). Generally, the methods of treatment include administering a therapeutically effective amount of composition that reduces at least one symptom of a disorder to a subject who is in need of, or who has been determined to be in need of such treatment.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 shows a schematic of the TGFRt15-TGFRs construct.

FIG. 2 shows an additional schematic of the TGFRt15-TGFRs construct.

FIG. 3 shows results of TGFβ1 inhibition by TGFRt15-TGFRs and TGFR-Fc.

FIG. 4 shows results of 32Dβ cell proliferation assay with TGFRt15-TGFRs or recombinant IL-15

FIGS. 5A and 5B show results of detecting IL-15 and TGF□RII in TGFRt15-TGFRs with corresponding antibodies using ELISA.

FIG. 6 is a line graph showing the chromatographic profile of TGFRt15-TGFRs protein containing cell culture supernatant following binding and elution on anti-TF antibody resin.

FIG. 7 shows the analytical SEC profile of TGFRt15-TGFRs.

FIG. 8 shows TGFRt15-TGFRs before and after deglycosylation as analyzed by reduced SDS-PAGE.

FIGS. 9A and 9B show spleen weight and the percentages of immune cell types in TGFRt15-TGFRs-treated and control-treated mice. FIG. 9A shows spleen weight in mice treated with TGFRt15-TGFRs as compared to PBS control. FIG. 9B shows the percentage of CD4⁺ T cells, CD8⁺ T cells, and NK cells in mice treated with TGFRt15-TGFRs as compared to PBS control.

FIGS. 10A and 10B show the spleen weight and immunostimulation over 92 hours in mice treated with TGFRt15-TGFRs. FIG. 10A shows spleen weight of mice treated with TGFRt15-TGFRs at 16, 24, 48, 72, and 92 hours after treatment. FIG. 10B shows the percentages of immune cells in mice treated with TGFRt15-TGFRs at 16, 24, 48, 72, and 92 hours after treatment.

FIGS. 11A and 11B show Ki67 and Granzyme B expression in mice treated with TGFRt15-TGFRs over time.

FIG. 12 shows enhancement of cytotoxicity of splenocytes by TGFRt15-TGFRs in C57BL/6 Mice.

FIG. 13 shows changes in tumor size in response to PBS treatment, chemotherapy alone, TGFRt15-TGFRs alone, or chemotherapy and TGFRt15-TGFRs combination, in a pancreatic cancer mouse model.

FIG. 14 shows the cytotoxicity of NK cells isolated from mice treated with TGFRt15-TGFRs.

FIGS. 15A-15B show the results of immunostimulation of an exemplary multi-chain polypeptide in C57BL/6 mice. FIG. 15A shows the spleen weight of mice treated with increasing dosage of the exemplary multi-chain polypeptide as compared to mice treated with the control solution. FIG. 15B shows the percentages of immune cell types present in the spleen of mice treated with increasing dosage of the exemplary multi-chain polypeptide as compared to mice treated with the control solution.

FIGS. 16A-16B show the duration of immunostimulation of an exemplary multi-chain polypeptide in C57BL/6 mice. FIG. 16A shows the spleen weight over a period of 92 hours in mice treated with 3 mg/kg of the exemplary multi-chain polypeptide.

FIG. 16B shows the percentages of immune cell types present in the spleen over a period of 92 hours in mice treated with 3 mg/kg of the exemplary multi-chain polypeptide.

FIGS. 17A-17B show the expression of Ki67 and Granzyme B in immune cells induced by the exemplary multi-chain polypeptide. FIG. 17A shows the expression of Ki67 in CD4⁺ T cells, CD8⁺ T cells, natural killer (NK) cells, and CD19⁺ B cells at various time points post-treatment with the multi-chain polypeptide. FIG. 17B shows the expression of Granzyme B in CD4⁺ T cells, CD8⁺ T cells, natural killer (NK) cells, and CD19⁺ B cells at various time points post-treatment with the multi-chain polypeptide.

FIG. 18 shows the effect of tumor inhibition by splenocytes prepared from mice treated with an exemplary multi-chain polypeptide at various time points after treatment.

FIGS. 19A and 19B show the percentages and the proliferation rate of CD4⁺ T cells, CD8⁺ T cells, Natural Killer (NK) cells, and CD19⁺ B cells in the blood of B6.129P2-ApoE^{tm1Unc}/J mice (purchased from The Jackson Laboratory) fed a control diet, a high fat diet and untreated, and mice fed a high fat diet and treated with TGFRt15-TGFRs, 2t2, or 21t15-TGFRs. FIG. 19A shows the percentages of the different cell types in each control and experimental group. FIG. 19B shows the proliferation rate of the of the different cell types in each control and experimental group.

FIGS. 20A-20E show exemplary physical appearance of mice fed either a control or high fat diet and were either untreated or treated with TGFRt15-TGFRs, 2t2, or 21t15-TGFRs.

FIG. 21 shows the fasting body weight of mice fed either a control or a high fat diet and were either untreated or treated with TGFRt15-TGFRs, 2t2, or 21t15-TGFRs.

FIG. 22 shows the fasting blood glucose levels of mice fed either a control or a high fat diet and were either untreated or treated with TGFRt15-TGFRs, 2t2, or 21t15-TGFRs.

FIGS. 23A-23F show chemotherapy-induced senescent B16F10 cells and expression of senescent genes. FIG. 23A shows chemotherapy induction of senescent B16F10 cells visualized using SA β-gal staining. FIGS. 23B-23F show expression of p21^{CIP1}, IL6, DPP4, RATE1E, and ULBP1 over time in the chemotherapy-induced senescent B16F10 cells.

FIGS. 24A-24F show colony formation and expression of stem cell markers by chemotherapy-induced senescent B16F10 cells. FIG. 24A shows colony formation by chemotherapy-induced senescent B16F10 cells. FIGS. 24B and 24C show expression of Oct4 mRNA and Notch4 mRNA by chemotherapy-induced senescent B16F10 cells as compared to control B16F10 cells. FIGS. 24D-24F show percentage of chemotherapy-induced senescent B16F10 cells double-positive for two out of the three stem cell markers including CD44, CD24, and CD133.

FIGS. 25A-25C show migratory and invasive properties of chemotherapy-induced senescent B16F10 cells. FIG. 25A shows the results of a migration assay comparing chemotherapy-induced senescent cells with stem cell properties (B16F10-SNC-CSC) with control B16F10 cells. FIGS. 25B and 25C show the results of an invasion assay comparing chemotherapy-induced senescent cells with stem cell properties (B16F10-SNC-CSC) with control B16F10 cells.

FIGS. 26A and 26B show in vitro expanded NK cells and their cytotoxicity against chemotherapy-induced senescent cells with stem cell properties (B16F10-SNC-CSC) or control B16F10 cells. FIG. 26A shows an exemplary schematic of a process of obtaining in vitro expanded NK cells. FIG. 26B shows cytotoxicity of the expanded NK cells against chemotherapy-induced senescent cells with stem cell properties (B16F10-SNC-CSC) or control B16F10 cells.

FIGS. 27A-27C show results of combination treatment using a mouse melanoma model. FIG. 27A shows an exemplary schematic for treating melanoma in a mouse model. FIGS. 27B and 27C show the change in tumor volume over time with combination treatments including TGFRt15-TGFRs as compared to chemotherapy or TA99 treatment alone.

FIG. 28A-28C are a set of graphs showing immunostimulation in C57BL/6 mice following treatment with 2t2.

FIGS. 29A and 29B are a set of graphs showing immunostimulation in C57BL/6 mice following treatment with TGFRt15-TGFRs.

FIGS. 30A-30C are a set of graphs showing in vivo stimulation of Tregs, NK cells, and CD8⁺ T cells in ApoE^{-/-} mice fed with a Western diet and treated with TGFRt15-TGFRs or 2t2.

FIGS. 31A and 31B are a set of graphs showing induction of splenocyte proliferation by 2t2 in C57BL/6 mice.

FIGS. 32A-32C are a set of graphs showing immunostimulation in C57BL/6 mice following treatment with TGFRt15-TGFRs.

FIGS. 33A and 33B are a set of graphs showing in vivo induction of proliferation of NK cells and CD8⁺ T cells in ApoE^{-/-} mice fed with a Western diet and treated with TGFRt15-TGFRs or 2t2.

FIG. 34 is a schematic and a set of graphs showing the persistence of 7t15-21s and anti-TF antibody-expanded NK cells in NSG mice following treatment with 7t15-21, TGFRt15-TGFRs or 2t2.

FIGS. 35A and 35B are a set of graphs showing enhancement of cytotoxicity of NK cells following treatment of NK cells with TGFRt15-TGFRs.

FIGS. 36A and 36B are a set of graphs showing enhancement of ADCC activity of NK cells following treatment of NK cells with TGFRt15-TGFRs.

FIG. 37 is a graph of in vitro killing of senescent B16F10 melanoma cells by TGFRt15-TGFRs/2t2-activated mouse NK cells.

FIGS. 38A-38H are a set of graphs showing antitumor activity of TGFRt15-TGFRs plus anti-TRP1 antibody (TA99) in combination with chemotherapy in a melanoma mouse model.

FIGS. 39A-39C are a set of graphs showing amelioration of the Western diet-induced hyperglycemia in ApoE^{-/-} mice by 2t2.

FIG. 40 shows upregulation of CD44 memory T cells. The upper panel shows upregulation of CD44 memory T cells upon treatment with TGFRt15-TGFRs. The lower panel shows upregulation of CD44 memory T cells upon treatment with 2t2.

FIG. 41 is a set of graphs showing immune-phenotype and cell proliferation following treatment with IL-15-based agents at day 3 post treatment.

FIGS. 42A-42C are graphs showing TGFRt15-TGFRs treatment reduces senescence-associated gene expression in C57BL/6 mice. The graphs show expression of p21^{CIP1}p21 and CD26 in lung (42A and 42B) and p21^{CIP1}p21 in liver (42C) tissues respectively.

FIG. 43 is a set of graphs showing CD4⁺, CD8⁺, and Treg cell percentages and proliferation.

FIG. 44 is a set of graphs showing NK, CD19+ and monocyte cell percentages and proliferation.

FIGS. 45A-45C are graphs showing evaluation of senescence markers p21^{CIP1}p21 and CD26 in lung and liver tissues. FIGS. 208A and 208B show lung p21^{CIP1}p21 (45A) and lung CD26 (45B) senescence markers. FIG. 45C shows liver p21^{CIP1}p21 senescence marker.

FIG. 46 is a set of graphs showing the immune-phenotype from peripheral blood analysis after 4 days post single dose treatment with TGFRt15-TGFRs.

FIG. 47 is a set of graphs showing the immune-phenotype from peripheral blood analysis after 4 days post single dose treatment with TGFRt15-TGFRs.

FIG. 48 is a graph showing β-Gal staining analysis by FACS at seven days after the second administration with TGFRt15-TGFRs.

FIG. 49 is a set of graphs showing the levels of senescence markers in liver tissue determined using qPCR at 7 days after the second administration with TGFRt15-TGFRs.

FIG. 50 is a set of graphs showing the levels of senescence markers in kidney tissue determined using qPCR at 7 days after the second administration with TGFRt15-TGFRs.

FIG. 51 is a set of graphs showing the levels of senescence markers in skin tissue determined using qPCR at 7 days after the second administration with TGFRt15-TGFRs.

FIG. 52 is a set of graphs showing the levels of senescence markers in lung tissue determined using qPCR at 7 days after the second administration with TGFRt15-TGFRs.

FIG. 53 is a set of histological images showing β-Gal staining on kidney tissue at 7 days post second treatment with TGFRt15-TGFRs.

FIG. 54 shows a schematic diagram of the interaction between the exemplary TGFβRII/IL-15RαSu and TGFβRII/TF/IL-15Mut proteins resulting in TGFRt15*-TGFRs complex.

FIG. 55 shows a schematic diagram of the interaction between the exemplary TGFβRII/IL-15RαSu and TGFβRII/TF/IL-15Mut proteins.

FIG. 56A is a graph showing the binding activity of TGFRt15-TGFRs to TGF-β1 and LAP.

FIG. 56B is a graph showing the binding activity of TGFRII/Fc to TGF-β1 and LAP.

FIG. 56C is a graph showing the binding activity of TGFRt15-TGFRs to TGF-1 and LAP.

FIG. 56D is a graph showing the binding activity of TGFRt15*-TGFRs to TGF-1 and LAP.

FIG. 56E is a graph showing the binding activity of TGFRt15-TGFRs, TGFRt15*-TGFRs, and 7t15-21s to CTLL-2 cells.

FIG. 57A is a graph of TGF-β1 blocking activity of TGFRt15-TGFRs and TGFRt15*-TGFRs.

FIG. 57B is a graph of the IL-15 biological activity of TGFRt15-TGFRs and TGFRt15*-TGFRs.

FIG. 57C is a graph showing that TGF-β1, TGF-β2, and TGF-β3 each similarly inhibit IL-4-induced CTLL-2 growth in the absence of TGFRt15*-TGFRs.

FIG. 57D is a graph showing that TGFRt15*-TGFRs significantly reversed the inhibition of TGF-1 and TGF-3 of IL-4-induced CTLL-2 cell growth.

FIG. 58A shows that there is no significant damage to the IL-15 domain of TGFRt15-TGFRs following 10-day incubation 4° C., 25° C., or 37° C.

FIG. 58B shows that there is no significant damage to the TGFβ-RII domain of TGFRt15-TGFRs following 10-day incubation 4° C., 25° C., or 37° C.

FIG. 58C is a graph showing TGF-β1 neutralizing activity of TGFRt15-TGFRs following incubation in human serum for 10 days at 4° C., 25° C., or 37° C.

FIG. 58D is a graph showing IL-15 activity of TGFRt15-TGFRs following incubation in human serum for 10 days at 4° C., 25° C., or 37° C.

FIG. 59A is a graph showing cell-mediated cell cytotoxicity in an assay using NK cells and the constructs shown.

FIG. 59B is a graph showing cell-mediated cell cytotoxicity in an assay using PMBCs and the constructs shown.

FIG. 59C is a graph showing intracellular granzyme B production in an assay using NK cells and the constructs shown.

FIG. 59D is a graph showing intracellular granzyme B production in an assay using PBMCs and the constructs shown.

FIG. 59E is a graph showing interferon-gamma production in an assay using NK cells and the constructs shown.

FIG. 59F is a graph showing interferon-gamma production in an assay using PMBCs and the constructs shown.

FIG. 60 is a graph showing the pharmacokinetics (half-life, t_{1/2}) of TGFRt15-TGFRs evaluated in female C57BL/6 mice.

FIG. 61 is a graph showing toxicity of TGFRt15-TGFRs in C57BL/6 mice.

FIG. 62 is a graph showing antitumor activity of TGFRt15-TGFRs in a C57BL/6 murine melanoma model.

FIG. 63 shows activity of TGFRt15-TGFRs in nine-week old C57BL6/j male mice, wherein the mice were given 50 μl of bleomycin (2.5 mg/kg, single dose) through the oropharyngeal route and then were given TGFRt15-TGFRs subcutaneously (3 mg/kg) on day 17 following bleomycin treatment.

FIG. 64 shows fasting plasma glucose levels in db/db mice 4 days post treatment with TGFRt15-TGFRs or TGFRt15*-TGFRs.

FIGS. 65A-65C show TGFβ1-3 levels in db/db mice 4 days post treatment with TGFRt15-TGFRs or TGFRt15*-TGFRs: TGFβ1 (FIG. 65A), TGFβ2 (FIG. 65B), and TGFβ3 (FIG. 65C).

FIGS. 66A-66E show lymphocyte subsets in db/db mice 4 days post treatment with TGFRt15-TGFRs or TGFRt15*-TGFRs: blood NK cells (FIG. 66A), blood Ki67+NK cells (FIG. 66B), blood granzyme B⁺ (GzmB⁺) (FIG. 66C), blood CD8⁺ (FIG. 66D), and blood CD8⁺Ki67⁺ T cells (FIG. 66E).

FIG. 67A shows the interaction of TGFRt15*-TGFRs or TGFRt15-TGFRs with latent TGFβ1 (SLC) or with CD39 (control).

FIG. 67B shows the interaction of TGFRt15*-TGFRs and TGFRII-Fc with latent TGFβ1.

FIG. 68 is a graph showing the clotting time of Innovin in the PT assay.

FIG. 69 is a graph showing the clotting time of TGFRt15-TGFRs in the PT assay.

FIG. 70 is a set of graphs showing gene expression of senescence markers PAI-1, IL-1α, IL6, and IL-1β in kidney and comparing young vs PBS or TGFRt15-TGFRs treated aged mice with short term vs long term follow-up.

FIG. 71 is a set of graphs showing gene expression of senescence markers IL-1a and IL6 in liver.

FIG. 72 shows protein expression of senescence marker PAI-1 in kidney.

FIG. 73 is a set of graphs showing that IL15SA (positive control) or TGFRt15*-TGFRs+IL15SA mediated an increase in the percentages of CD3⁺CD8⁺, CD3⁻NK1.1⁺, and CD3⁺CD45⁺ immune cells in the blood, whereas treatment with TGFRt15*-TGFRs had little or no effect on the percentage of these cell populations.

FIG. 74 is a set of graphs showing that IL15SA (positive control) or TGFRt15*-TGFRs+IL15SA mediated an increase in the percentages of CD3⁺CD8⁺, CD3⁻NK1.1⁺, and CD3⁺CD45⁺ immune cells in the spleen, whereas treatment with TGFRt15*-TGFRs had little or no effect on the percentage of these cell populations.

FIG. 75A shows gene expression of senescence marker p21, in kidney and liver tissues, post test article treatment.

FIG. 75B shows gene expression of senescence marker PAI1, in kidney and liver tissues, post study treatment.

FIG. 75C shows gene expression of senescence marker IL-1α, in kidney and liver tissues, post study treatment.

FIG. 75D shows gene expression of senescence marker IL-6, in kidney and liver tissues, post study treatment.

FIG. 76A shows CD4⁺, CD8⁺, and Treg cell percentages and proliferation following treatment with the agents shown.

FIG. 76B shows NK, CD19⁺, and monocyte cell percentages and proliferation following treatment with the agents shown.

FIG. 77A shows evaluation of gene expression of senescence markers p21 in lung tissue of mice following chemotherapy and treatment with the agents shown.

FIG. 77B shows evaluation of gene expression of senescence marker CD26 in lung tissue of mice following chemotherapy and treatment with the agents shown.

FIG. 77C shows evaluation of gene expression of senescence marker p21 in liver tissue of mice following chemotherapy and treatment with the agents shown.

FIGS. 78A and 78B are graphs showing TGFRt15-TGFRs treatment enhances the immune cell proliferation, expansion and activation in the peripheral blood of B16F10 tumor bearing mice.

FIG. 79 is a set of graphs showing TGFRt15-TGFRs treatment decreases levels of TGFβ in the plasma of B16F10 tumor bearing mice.

FIG. 80 is a set of graphs showing TGFRt15-TGFRs treatment reduces levels of proinflammatory cytokines in the plasma of B16F10 tumor bearing mice.

FIG. 81 shows TGFRt15-TGFRs treatment enhances NK and CD8 expansion in the spleen of B16F10 tumor bearing mice.

FIGS. 82A and 82B show TGFRt15-TGFRs treatment enhances glycolytic activity of splenocytes in B16F10 tumor bearing mice.

FIGS. 83A and 83B show TGFRt15-TGFRs treatment enhances mitochondrial respiration of splenocytes in B16F10 tumor bearing mice.

FIGS. 84A and 84B show TGFRt15-TGFRs treatment enhances NK and CD8 immune cell infiltration (TILs) into tumors of B16F10 tumor bearing mice.

FIG. 85 shows histopathological analysis of tumors following TGFRt15-TGFRs treatment, wherein following TGFRt15-TGFRs+TA99 antibody treatment, tumors displayed less mitotic and necrotic activity. The mitotic index is correlated to the dividing cells and presence of necrosis is a measure of more aggressive features and poor prognosis.

FIG. 86 is a graph showing anti-PD-L1 antibody in combination with TGFRt15-TGFRs+TA99 antibody and chemotherapy in B16F10 melanoma mouse model.

FIG. 87 is a graph showing that anti-tumor efficacy of TGFRt15-TGFRs in B16F10 melanoma mouse model is dependent on NK and CD8 T cells.

FIGS. 88A and 88B are graphs showing gene expression of senescence markers p21, IL-1a and IL6 in liver and lung tissues of tumor bearing mice following chemotherapy.

FIG. 89 is a graph showing induction of gene expression of senescence markers p21, IL6, H2AX, and NK cell ligands, Rae1e and ULBP1 by docetaxel treatment of B16F10 GFP cells.

FIG. 90 shows tumor infiltrating lymphocytes/day after 4 days post treatment in tumor bearing mice.

FIGS. 91A and 91B show flow cytometry analysis on tumor cells indicating that mice which received immunotherapy treatment showed lower number of GFP positive senescent tumor cells post 4 days and 10 days of treatment as compared to the PBS control group (FIG. 91A), and tumor cells plated in 24 well plate evaluated by fluorescence microscopy (FIG. 91B).

FIG. 92 shows TGFβ levels in kidney of mice after inducing kidney injury with cisplatin and treatment with TGFRt15-TGFRs.

FIGS. 93A-93C show the toxicological effects of repeat dose subcutaneous administration of TGFRt15-TGFRs in C57BL/6 mice. Changes in body weights are shown through SD21 (FIG. 93A). Spleen weights (FIG. 93B) and blood cells counts and differentials (FIG. 93C) are indicated for mice at SD7 after one dose and SD21 after two doses of TGFRt15-TGFRs.

FIG. 94 shows plasma levels of TGF-β isoforms in mice after in vivo treatment with PBS, TGFRt15-TGFRs (3 mg/kg) or TGFRt15*-TGFRs (3 mg/kg).

FIGS. 95A and 95B show the changes in rates of glycolytic capacity (ECAR) (FIG. 95A) and mitochondrial respiratory capacity (OCR) (FIG. 95B) in splenocytes of mice following in vivo treatment with PBS, TGFRt15-TGFRs, TGFRt15*-TGFRs or IL15SA.

FIGS. 96A and 96B show the changes in rates of glycolytic capacity (ECAR) (FIG. 96A) and mitochondrial respiratory capacity (OCR) (FIG. 96B) in mouse splenocytes following in vitro treatment with PBS, TGFRt15-TGFRs, or TGFRt15*-TGFRs.

FIGS. 97A-97E show the changes in tumor growth and survival of B16F10 melanoma tumors in C57BL/6 mice following in vitro treatment with PBS, TGFRt15-TGFRs, or TGFRt15*-TGFRs. Tumor volume (FIG. 97A) and mouse survival (based on tumor volume<4000 mm³) (FIG. 97B) were assessed. Mice were intraperitoneally treated with anti-CD8, anti-NK, or anti-CD8 and anti-NK Abs for 1 week to deplete immune cells prior to injection with B16F10 melanoma tumor cells as in FIG. 97A. Tumor bearing mice were then treated with PBS or 20 mg/kg TGFRt15-TGFRs on day 1 and 4 post-tumor cell inoculation. Tumor volume of animals (FIG. 97C) and mouse survival (FIG. 97D) were assessed. B16F10 tumor bearing mice were treated with PBS or 20 mg/kg of TGFRt15-TGFRs on day 1 and 7 post-tumor inoculation (FIG. 97E). On day 11 post tumor inoculation, tumors were collected and tumor-infiltrating NK1.1⁺ cells and CD8⁺ T cells were quantitated by flow cytometry.

FIG. 98A shows the fold change in gene expression levels in pancreas of db/db mice receiving TGFRt15-TGFRs compared to PBS control.

FIGS. 98B-98D show the average fold change in pancreatic expression levels for genes of the SASP, Aging and Beta cell indices, respectively, for db/db mice receiving TGFRt15-TGFRs compared to PBS control.

FIGS. 99A and 99B show multispectral imaging of pancreatic tissue sections from db/db mice treated with PBS (control) (FIG. 99A) or TGFRt15-TGFRs (FIG. 99B). A representative pancreatic islet is shown, insulin⁺ islet beta cells as OPAL-520, insulin⁺p21⁺ beta cells as OPAL-570 (seen as white cells in gray-scale image) was reduced in TGRt15-TGFRs treated group (FIG. 99B) compared to PBS treated group (FIG. 99A).

FIGS. 99C and 99D show levels of islet insulin⁺ (FIG. 99C) and islet insulin⁺p21⁺ (FIG. 99D) cells in pancreatic tissue sections from db/db mice treated with PBS (control) or TGFRt15-TGFRs.

FIGS. 100A-100C show treatment effects on the percentage of blood immune cell subsets in db/db mice receiving PBS (control) or TGFRt15-TGFRs.

FIG. 101 shows the percentage of Ki67 positive immune cells induced in the blood following subcutaneous treatment of Cynomolgus monkeys with TGFRt15-TGFRs compared to PBS (vehicle).

FIG. 102 shows the extracellular acidification rate (ECAR) representing glycolytic function of splenocytes isolated from young (6-week-old) and aged (72-week-old) mice 4 days after in vivo treatment with PBS, TGFRt15-TGFRs (3 mg/kg) or TGFRt15*-TGFRs (3 mg/kg).

FIG. 103 shows the oxygen consumption rate (OCR) representing mitochondrial respiration of splenocytes isolated from young (6-week-old) and aged (72-week-old) mice 4 days after in vivo treatment with PBS, TGFRt15-TGFRs (3 mg/kg) or TGFRt15*-TGFRs (3 mg/kg).

FIG. 104 shows the percentages of immune cell subsets in the blood of young (6-week-old) and aged (72-week-old) mice 4 days after in vivo treatment with PBS, TGFRt15-TGFRs (3 mg/kg) or TGFRt15*-TGFRs (3 mg/kg).

FIG. 105 shows the percentages of immune cell subsets in the spleen of young (6-week-old) and aged (72-week-old) mice 4 days after in vivo treatment with PBS, TGFRt15-TGFRs or TGFRt15*-TGFRs.

FIG. 106 shows gene expression levels for IL1-α, IL1-β, IL-6, p21 and PAI-1 in liver of aged mice after one or two doses of TGFRt15-TGFRs treatment.

FIG. 107 shows the inflammation score of liver tissues of aged mice after one or two doses of TGFRt15-TGFRs treatment.

FIG. 108 shows expression levels of IL1-α, IL1-β, IL-6, IL-8, TGF-β, PAI-1, collagen and fibronectin protein in liver of aged mice after with one or two doses treatment of TGFRt15-TGFRs.

FIG. 109 shows the levels of β-galactosidase in liver tissues of aged mice 4 days after in vivo treatment with PBS or TGFRt15-TGFRs.

FIG. 110 shows the survival curves of 72-week-old C57BL/6 mice following subcutaneous treatment with PBS or one dose of TGFRt15-TGFRs (3 mg/kg).

FIG. 111 shows protein levels of SASP factors in livers of B16F10 tumor-bearing mice following chemotherapy and TGFRt15-TGFRs+TA99 therapy.

FIGS. 112A and 112B show effects of CD8⁺ T cells (dpCD8) and NK cell (dpNK) antibody depletion on the levels of TIS B16F10-GFP cells (FIG. 112A) and NK and CD8⁺ T cells (FIG. 112B) in the tumors of mice following chemotherapy and TGFRt15-TGFRs+TA99 therapy.

FIGS. 113A-113E show the anti-tumor activity and mechanism of action of TGFRt15-TGFRs+TA99 in combination with immune checkpoint inhibitor in B16F10 tumor-bearing mice. FIG. 113A shows an exemplary schematic for treating B16F10 melanoma in a mouse model. FIG. 113B shows the change in tumor volume over time and at day 18 following combination treatments including TGFRt15-TGFRs+TA99+anti-PD-L1 antibody following doxetaxel as compared to PBS or chemotherapy treatment alone. FIGS. 113C and 113D show treatment effects on the percentages of tumor infiltrating CD28⁺CD8⁺ T cells and splenic IFNγ CD8⁺ T cells on day 18. FIG. 113E shows treatment effects on the levels (MFI) of NKG2D of tumor infiltrating CD8⁺ and CD8⁺CD44^{hi} T cells on day 18.

FIGS. 114A-114D show the changes in tumor growth and survival of SW1990 human pancreatic tumors in C57BL/6 scid mice following in vitro treatment with PBS, gemcitabine and nab-paclitaxel chemotherapy, TGFRt15-TGFRs, or TGFRt15-TGFRs+chemotherapy. FIG. 114A shows an exemplary schematic for treating SW1990 human pancreatic tumors in a xenograft mouse model. FIGS. 114B and 114C show the change in tumor volume over time and at day 38, respectively, following combination treatments including TGFRt15-TGFRs+chemotherapy as compared to PBS or chemotherapy treatment alone. FIG. 114D shows treatment effects on survival of mice bearing SW1990 human pancreatic tumors.

FIGS. 115A-115C are a set of graphs showing levels of gene expression of senescence markers (IL-1α, IL-6, and PAI-1, respectively) in tissues of aged mice following treatment with PBS; TGFRt15-TGFRs; 2t2; first dose TGFRt15-TGFRs at day 0 with second dose 2t2 at day 60; or first dose 212 at day 0 with second dose TGFRt15-TGFRs at day 60.

FIG. 116 is an exemplary schematic of the experimental design using a melanoma mouse model.

FIGS. 117A-117H are graphs showing the effect of administration of TGFRt15-TGFRs on NK/T cell proliferation, expansion, and activation in the blood of the melanoma mouse model.

FIGS. 118A-118C are graphs showing the effect of TGFRt15-TGFRs treatment on TGF-β1, TGF-β2, and TGF-β3 levels in the plasma of the melanoma mouse model.

FIGS. 119A-119E are graphs showing the effect of treatment with dexamethasone or a combination of TGFRt15-TGFRs and dexamethasone on plasma levels of IL-2, IL-1β, IL-6, and GM-CSF in the melanoma mouse model.

FIGS. 120A and 120B are graphs showing the effect of treatment with dexamethasone or a combination of TGFRt15-TGFRs and dexamethasone on the levels of NK cells and CD8⁺ T-cells in the spleens of the melanoma mouse model.

FIGS. 121A-121C are a set of graphs showing the effect of treatment with saline (black line), dexamethasone (dark grey line), or a combination of dexamethasone, TGFRt15-TGFRs, and TA99 (light gray line) on the glycolytic activity of splenocytes.

FIGS. 122A-122L are a set of graphs the effect of treatment with saline, dexamethasone, or a combination of dexamethasone, TGFRt15-TGFRs, and TA99 on glycolytic activity (glycolysis, glycolytic capacity, glycolytic reserve, and non-glycolytic acidification) of splenocytes from a melanoma mouse model.

FIGS. 123A-123C are a set of graphs showing the effect of treatment with PBS, dexamethasone, or a combination of dexamethasone, TGFRt15-TGFRs, and TA99 on mitochondrial respiration of splenocytes from a melanoma mouse model.

FIGS. 124A-124L are a set of graphs showing the effect of treatment with PBS, dexamethasone, or a combination of dexamethasone, TGFRt15-TGFRs, and TA99 on mitochondrial respiration of splenocytes (basal respiration, maximal respiration, spare respiratory capacity, and ATP production) from a mouse melanoma model.

FIGS. 125A-125H are a set of graphs showing the effect of treatment with PBS, dexamethasone, or a combination of dexamethasone, TGFRt15-TGFRs, and TA99 on the infiltration of NK/Ki67 cells, CD8/Ki67 cells, NK cells, CD8 cells, NK/CD25 cells, NK/Granzyme B cells, CD8/CD25 cells, and CD8/Granzyme B cells into melanoma tumors in a melanoma mouse model.

FIG. 126A is a schematic of the experimental design for therapy-induced senescence in B16F10 tumors in a melanoma mouse model.

FIGS. 126B-126E are a set of graphs showing the effect of DTX treatment on senescence-associated gene expression (DPP4, IL-6, p16, and p21, respectively) in B16F10 tumor cells in the mice.

FIG. 127A is a schematic of the experimental design for therapy-induced senescence in B16F10 tumors in a melanoma mouse model.

FIGS. 127B and 127C are graphs showing the effect of treatment with saline, dexamethasone, or a combination of dexamethasone, TGFRt15-TGFRs, and TA99 on expression of p21 and IL-6, respectively in B16F10 tumors in a melanoma tumor model.

FIGS. 128A-128D are a set of graphs showing levels of protein expression of senescence markers (PAI1, IL-1α, CXCL1, and IL-2, respectively) in plasma of aged mice following treatment with PBS; TGFRt15-TGFRs; 2t2; first dose TGFRt15-TGFRs at day 0 with second dose 2t2 at day 60; or first dose 2t2 at day 0 with second dose TGFRt15-TGFRs at day 60.

FIG. 129 shows RNA-seq analysis of differentially expressed genes between the PBS (control group) or TGFRt15-TGFRs (TGFRt15-TGFRs group) in the liver of db/db mice.

FIG. 130 shows RNA-seq analysis of differentially expressed genes between the PBS (control group) or TGFRt15-TGFRs (TGFRt15-TGFRs group) in aged mice liver.

### DETAILED DESCRIPTION

Provided herein are methods of treating unresectable advanced/metastatic pancreatic cancer in a subject that include administering to the subject a therapeutically effective amount of a multi-chain chimeric polypeptide, wherein the multi-chain chimeric polypeptide that include (a) a first chimeric polypeptide including: (i) a first target-binding domain; (ii) a soluble tissue factor domain; and (iii) a first domain of a pair of affinity domains; (b) a second chimeric polypeptide including: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII.

Also provided herein are methods of improving the objective response rate in subjects having unresectable advanced/metastatic pancreatic cancer that include administering to the subjects a therapeutically effective amount of any of the multi-chain chimeric polypeptides described herein.

Also provided herein are methods of increasing progression-free survival or the progression-free survival rate in a subject or population of subjects having unresectable advanced/metastatic pancreatic cancer that include administering to the subject(s) a therapeutically effective amount of any of the multi-chain chimeric polypeptides described herein.

Also provided herein are methods of increasing time to progression in a subject or population of subjects having unresectable advanced/metastatic pancreatic cancer that include administering to the subject(s) a therapeutically effective amount of any of the multi-chain chimeric polypeptides described herein.

Also provided herein are methods of increasing duration of response in a subject or population of subjects having unresectable advanced/metastatic pancreatic cancer that include administering to the subject(s) a therapeutically effective amount of any of the multi-chain chimeric polypeptides described herein.

Also provided herein are methods of increasing overall survival in a population of subjects having unresectable advanced/metastatic pancreatic cancer that include administering to the subjects a therapeutically effective amount of any of the multi-chain chimeric polypeptides described herein.

In some examples of any of the multi-chain chimeric polypeptides described herein the total length of first chimeric polypeptide and/or the second chimeric polypeptide can each independently be about 50 amino acids to about 3000 amino acids, about 50 amino acids to about 2500 amino acids, about 50 amino acids to about 2000 amino acids, about 50 amino acids to about 1500 amino acids, about 50 amino acids to about 1000 amino acids, about 50 amino acids to about 800 amino acids, about 50 amino acids to about 600 amino acids, about 50 amino acids to about 500 amino acids, about 50 amino acids to about 450 amino acids, about 50 amino acids to about 400 amino acids, about 50 amino acids to about 350 amino acids, about 50 amino acids to about 300 amino acids, about 50 amino acids to about 250 amino acids, about 50 amino acids to about 200 amino acids, about 50 amino acids to about 150 amino acids, about 50 amino acids to about 100 amino acids, about 100 amino acids to about 3000 amino acids, about 100 amino acids to about 2500 amino acids, about 100 amino acids to about 2000 amino acids, about 100 amino acids to about 1500 amino acids, about 100 amino acids to about 1000 amino acids, about 100 amino acids to about 800 amino acids, about 100 amino acids to about 600 amino acids, about 100 amino acids to about 500 amino acids, about 100 amino acids to about 450 amino acids, about 100 amino acids to about 400 amino acids, about 100 amino acids to about 350 amino acids, about 100 amino acids to about 300 amino acids, about 100 amino acids to about 250 amino acids, about 100 amino acids to about 200 amino acids, about 100 amino acids to about 150 amino acids, about 150 amino acids to about 3000 amino acids, about 150 amino acids to about 2500 amino acids, about 150 amino acids to about 2000 amino acids, about 150 amino acids to about 1500 amino acids, about 150 amino acids to about 1000 amino acids, about 150 amino acids to about 800 amino acids, about 150 amino acids to about 600 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 250 amino acids, about 150 amino acids to about 200 amino acids, about 200 amino acids to about 3000 amino acids, about 200 amino acids to about 2500 amino acids, about 200 amino acids to about 2000 amino acids, about 200 amino acids to about 1500 amino acids, about 200 amino acids to about 1000 amino acids, about 200 amino acids to about 800 amino acids, about 200 amino acids to about 600 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 250 amino acids, about 250 amino acids to about 3000 amino acids, about 250 amino acids to about 2500 amino acids, about 250 amino acids to about 2000 amino acids, about 250 amino acids to about 1500 amino acids, about 250 amino acids to about 1000 amino acids, about 250 amino acids to about 800 amino acids, about 250 amino acids to about 600 amino acids, about 250 amino acids to about 500 amino acids, about 250 amino acids to about 450 amino acids, about 250 amino acids to about 400 amino acids, about 250 amino acids to about 350 amino acids, about 250 amino acids to about 300 amino acids, about 300 amino acids to about 3000 amino acids, about 300 amino acids to about 2500 amino acids, about 300 amino acids to about 2000 amino acids, about 300 amino acids to about 1500 amino acids, about 300 amino acids to about 1000 amino acids, about 300 amino acids to about 800 amino acids, about 300 amino acids to about 600 amino acids, about 300 amino acids to about 500 amino acids, about 300 amino acids to about 450 amino acids, about 300 amino acids to about 400 amino acids, about 300 amino acids to about 350 amino acids, about 350 amino acids to about 3000 amino acids, about 350 amino acids to about 2500 amino acids, about 350 amino acids to about 2000 amino acids, about 350 amino acids to about 1500 amino acids, about 350 amino acids to about 1000 amino acids, about 350 amino acids to about 800 amino acids, about 350 amino acids to about 600 amino acids, about 350 amino acids to about 500 amino acids, about 350 amino acids to about 450 amino acids, about 350 amino acids to about 400 amino acids, about 400 amino acids to about 3000 amino acids, about 400 amino acids to about 2500 amino acids, about 400 amino acids to about 2000 amino acids, about 400 amino acids to about 1500 amino acids, about 400 amino acids to about 1000 amino acids, about 400 amino acids to about 800 amino acids, about 400 amino acids to about 600 amino acids, about 400 amino acids to about 500 amino acids, about 400 amino acids to about 450 amino acids, about 450 amino acids to about 3000 amino acids, about 450 amino acids to about 2500 amino acids, about 450 amino acids to about 2000 amino acids, about 450 amino acids to about 1500 amino acids, about 450 amino acids to about 1000 amino acids, about 450 amino acids to about 800 amino acids, about 450 amino acids to about 600 amino acids, about 450 amino acids to about 500 amino acids, about 500 amino acids to about 3000 amino acids, about 500 amino acids to about 2500 amino acids, about 500 amino acids to about 2000 amino acids, about 500 amino acids to about 1500 amino acids, about 500 amino acids to about 1000 amino acids, about 500 amino acids to about 800 amino acids, about 500 amino acids to about 600 amino acids, about 600 amino acids to about 3000 amino acids, about 600 amino acids to about 2500 amino acids, about 600 amino acids to about 2000 amino acids, about 600 amino acids to about 1500 amino acids, about 600 amino acids to about 1000 amino acids, about 600 amino acids to about 800 amino acids, about 800 amino acids to about 3000 amino acids, about 800 amino acids to about 2500 amino acids, about 800 amino acids to about 2000 amino acids, about 800 amino acids to about 1500 amino acids, about 800 amino acids to about 1000 amino acids, about 1000 amino acids to about 3000 amino acids, about 1000 amino acids to about 2500 amino acids, about 1000 amino acids to about 2000 amino acids, about 1000 amino acids to about 1500 amino acids, about 1500 amino acids to about 3000 amino acids, about 1500 amino acids to about 2500 amino acids, about 1500 amino acids to about 2000 amino acids, about 2000 amino acids to about 3000 amino acids, about 2000 amino acids to about 2500 amino acids, or about 2500 amino acids to about 3000 amino acids.

In some embodiments of any of the multi-chain chimeric polypeptides described herein, the first target-binding domain (e.g., any of the first target-binding domains described herein) and the soluble tissue factor domain (e.g., any of the exemplary soluble tissue factor domains described herein) directly abut each other in the first chimeric polypeptide. In some embodiments of any of the multi-chain chimeric polypeptides described herein, the first chimeric polypeptide further comprises a linker sequence (e.g., any of the exemplary linker sequences described herein or known in the art) between the first target-binding domain (e.g., any of the exemplary first target-binding domains described herein) and the soluble tissue factor domain (e.g., any of the exemplary soluble tissue factor domains described herein) in the first chimeric polypeptide.

In some embodiments of any of the multi-chain chimeric polypeptides described herein, the soluble tissue factor domain (e.g., any of the exemplary soluble tissue factor domains described herein) and the first domain of the pair of affinity domains (e.g., any of the exemplary first domains of any of the exemplary pairs of affinity domains described herein) directly abut each other in the first chimeric polypeptide. In some embodiments of any of the multi-chain chimeric polypeptides described herein, the first chimeric polypeptide further comprises a linker sequence (e.g., any of the exemplary linker sequences described herein or known in the art) between the soluble tissue factor domain (e.g., any of the exemplary soluble tissue factor domains described herein) and the first domain of the pair of affinity domains (e.g., any of the exemplary first domains of any of the exemplary pairs of affinity domains described herein) in the first chimeric polypeptide.

In some embodiments of any of the multi-chain chimeric polypeptides described herein, the second domain of the pair of affinity domains (e.g., any of the exemplary second domains of any of the exemplary pairs of affinity domains described herein) and the second target-binding domain (e.g., any of the exemplary second target-binding domains described herein) directly abut each other in the second chimeric polypeptide. In some embodiments of any of the multi-chain chimeric polypeptides described herein, the second chimeric polypeptide further comprises a linker sequence (e.g., any of the exemplary linker sequences described herein or known in the art) between the second domain of the pair of affinity domains (e.g., any of the exemplary second domains of any of the exemplary pairs of affinity domains described herein) and the second target-binding domain (e.g., any of the exemplary second target-binding domains described herein) in the second chimeric polypeptide.

Non-limiting aspects of these chimeric polypeptides, nucleic acids, vectors, cells, and methods are described below, and can be used in any combination without limitation. Additional aspects of these chimeric polypeptides, nucleic acids, vectors, cells, and methods are known in the art.

### Tissue Factor

Human tissue factor is a 263 amino-acid transmembrane protein containing three domains: (1) a 219-amino acid N-terminal extracellular domain (residues 1-219); (2) a 22-amino acid transmembrane domain (residues 220-242); and (3) a 21-amino acid cytoplasmic C-terminal tail (residues 242-263) ((UniProtKB Identifier Number: P13726). The cytoplasmic tail contains two phosphorylation sites at Ser253 and Ser258, and one S-palmitoylation site at Cys245. Deletion or mutation of the cytoplasmic domain was not found to affect tissue factor coagulation activity. Tissue factor has one S-palmitoylation site in the intracellular domain of the protein at Cys245. The Cys245 is located at the amino acid terminus of the intracellular domain and close to the membrane surface. The tissue factor transmembrane domain is composed of a single-spanning α-helix.

The extracellular domain of tissue factor, composed of two fibronectin type III domains, is connected to the transmembrane domain through a six-amino acid linker. This linker provides conformational flexibility to decouple the tissue factor extracellular domain from its transmembrane and cytoplasmic domains. Each tissue factor fibronectin type III module is composed of two overlapping β sheets with the top sheet domain containing three antiparallel β-strands and the bottom sheet containing four β-strands. The β-strands are connected by β-loops between strand βA and βB, βC and βD, and βE and βF, all of which are conserved in conformation in the two modules. There are three short α-helix segments connecting the β-strands. A unique feature of tissue factor is a 17-amino acid β-hairpin between strand β10 and strand β11, which is not a common element of the fibronectin superfamily. The N-terminal domain also contains a 12 amino acid loop between β6F and β7G that is not present in the C-terminal domain and is unique to tissue factor. Such a fibronectin type III domain structure is a feature of the immunoglobulin-like family of protein folds and is conserved among a wide variety of extracellular proteins.

The zymogen FVII is rapidly converted to FVIIa by limited proteolysis once it binds to tissue to form the active tissue factor-FVIIa complex. The FVIIa, which circulates as an enzyme at a concentration of approximately 0.1 nM (1% of plasma FVII), can also bind directly to tissue factor. The allosteric interaction between tissue factor and FVIIa on the tissue factor-FVIIa complex greatly increases the enzymatic activity of FVIIa: an approximate 20- to 100-fold increase in the rate of hydrolysis of small, chromogenic peptidyl substrates, and nearly a million-fold increase in the rate of activation of the natural macromolecular substrates FIX and FX. In concert with allosteric activation of the active site of FVIIa upon binding to tissue factor, the formation of tissue factor-FVIIa complex on phospholipid bilayer (i.e., upon exposure of phosphatidyl-L-serine on membrane surfaces) increases the rate of FIX or FX activation, in a Ca²⁺-dependent manner, an additional 1,000-fold. The roughly million-fold overall increase in FX activation by tissue factor-FVIIa-phospholipid complex relative to free FVIIa is a critical regulatory point for the coagulation cascade.

FVII is a ˜50 kDa, single-chain polypeptide consisting of 406 amino acid residues, with an N-terminal γ-carboxyglutamate-rich (GLA) domain, two epidermal growth factor-like domains (EGF1 and EFG2), and a C-terminal serine protease domain. FVII is activated to FVIIa by a specific proteolytic cleavage of the Ile-¹⁵⁴-Arg¹⁵² bond in the short linker region between the EGF2 and the protease domain. This cleavage results in the light and heavy chains being held together by a single disulfide bond of Cys¹³⁵ and Cys²⁶². FVIIa binds phospholipid membrane in a Ca²⁺-dependent manner through its N-terminal GLA-domain. Immediately C-terminal to the GLA domain is an aromatic stack and two EGF domains. The aromatic stack connects the GLA to EGF1 domain which binds a single Ca²⁺ ion. Occupancy of this Ca²⁺-binding site increases FVIIa amidolytic activity and tissue factor association. The catalytic triad consist of His¹⁹³, Asp²⁴², and Ser³⁴⁴, and binding of a single Ca²⁺ ion within the FVIIa protease domain is critical for its catalytic activity. Proteolytic activation of FVII to FVIIa frees the newly formed amino terminus at Ile¹⁵³ to fold back and be inserted into the activation pocket forming a salt bridge with the carboxylate of Asp³⁴³ to generate the oxyanion hole. Formation of this salt bridge is critical for FVIIa activity. However, oxyanion hole formation does not occur in free FVIIa upon proteolytic activation. As a result, FVIIa circulates in a zymogen-like state that is poorly recognized by plasma protease inhibitors, allowing it to circulate with a half-life of approximately 90 minutes.

Tissue factor-mediated positioning of the FVIIa active site above the membrane surface is important for FVIIa towards cognate substrates. Free FVIIa adopts a stable, extended structure when bound to the membrane with its active site positioned ˜80 Å above the membrane surface. Upon FVIIa binding to tissue factor, the FVa active site is repositioned ˜6 Å closer to the membrane. This modulation may aid in a proper alignment of the FVIIa catalytic triad with the target substrate cleavage site. Using GLA-domainless FVIIa, it has been shown that the active site was still positioned a similar distance above the membrane, demonstrating that tissue factor is able to fully support FVIIa active site positioning even in the absence of FVIIa-membrane interaction. Additional data showed that tissue factor supported full FVIIa proteolytic activity as long as the tissue factor extracellular domain was tethered in some way to the membrane surface. However, raising the active site of FVIIa greater than 80 Å above the membrane surface greatly reduced the ability of the tissue factor-FVIIa complex to activate FX but did not diminish tissue factor-FVIIa amidolytic activity.

Alanine scanning mutagenesis has been used to assess the role of specific amino acid side chains in the tissue factor extracellular domain for interaction with FVIIa (Gibbs et al., Biochemistry 33 (47): 14003-14010, 1994; Schullek et al., J Biol Chem 269 (30): 19399-19403, 1994). Alanine substitution identified a limited number of residue positions at which alanine replacements cause 5- to 10-fold lower affinity for FVIIa binding. Most of these residue side chains were found to be well-exposed to solvent in the crystal structure, concordant with macromolecular ligand interaction. The FVIIa ligand-binding site is located over an extensive region at the boundary between the two modules. In the C-module, residues Arg¹³⁵ and Phe¹⁴⁰ located on the protruding B-C loop provide an independent contact with FVIIa. Leu¹³³ is located at the base of the fingerlike structure and packed into the cleft between the two modules. This provides continuity to a major cluster of important binding residues consisting of Lys²⁰, Thr⁶⁰, Asp⁵⁸, and Ile²². Thr⁶⁰ is only partially solvent-exposed and may play a local structural role rather than making a significant contact with ligand. The binding site extends onto the concave side of the intermodule angle involving Glu²⁴ and Gln¹¹⁰, and potentially the more distant residue Val²⁰⁷. The binding region extends from Asp⁵⁸ onto a convex surface area formed by Lys⁴⁸, Lys⁴⁶, Gln³⁷, Asp⁴⁴, and Trp⁴⁵. Trp⁴⁵ and Asp⁴⁴ do not interact independently with FVIIa, indicating that the mutational effect at the Trp⁴⁵ position may reflect a structural importance of this side chain for the local packing of the adjacent Asp⁴⁴ and Gln³⁷ side chain. The interactive area further includes two surface-exposed aromatic residues, Phe⁷⁶ and Tyr⁷⁸, which form part of the hydrophobic cluster in the N-module.

The known physiologic substrates of tissue factor-FVIIa are FVII, FIX, and FX and certain proteinase-activated receptors. Mutational analysis has identified a number of residues that, when mutated, support full FVIIa amidolytic activity towards small peptidyl substrates but are deficient in their ability to support macromolecular substrate (i.e., FVII, FIX, and FX) activation (Ruf et al., J Biol Chem 267 (31): 22206-22210, 1992; Ruf et al., J Biol Chem 267 (9): 6375-6381, 1992; Huang et al., J Biol Chem 271 (36): 21752-21757, 1996; Kirchhofer et al., Biochemistry 39 (25): 7380-7387, 2000). The tissue factor loop region at residues 159-165, and residues in or adjacent to this flexible loop have been shown to be critical for the proteolytic activity of the tissue factor-FVIIa complex. This defines the proposed substrate-binding exosite region of tissue factor that is quite distant from the FVIIa active site. A substitution of the glycine residue by a marginally bulkier residue alanine, significantly impairs tissue factor-FVIIa proteolytic activity. This suggests that the flexibility afforded by glycine is critical for the loop of residues 159-165 for tissue factor macromolecular substrate recognition.

The residues Lys¹⁶⁵ and Lys¹⁶⁶ have also been demonstrated to be important for substrate recognition and binding. Mutation of either of these residues to alanine results in a significant decrease in the tissue factor co-factor function. Lys¹⁶⁵ and Lys¹⁶⁶ face away from each other, with Lys¹⁶⁵ pointing towards FVIIa in most tissue factor-FVIIa structures, and Lys¹⁶⁶ pointing into the substrate binding exosite region in the crystal structure. Putative salt bridge formation between Lys¹⁶⁵ of and Gla³⁵ of FVIIa would support the notion that tissue factor interaction with the GLA domain of FVIIa modulates substrate recognition. These results suggest that the C-terminal portion of the tissue factor ectodomain directly interacts with the GLA-domain, the possible adjacent EGF1 domains, of FIX and FX, and that the presence of the FVIIa GLA-domain may modulate these interactions either directly or indirectly.

### Soluble Tissue Factor Domain

In some embodiments of any of the polypeptides, compositions, or methods described herein, the soluble tissue factor domain can be a wildtype tissue factor polypeptide lacking the signal sequence, the transmembrane domain, and the intracellular domain. In some examples, the soluble tissue factor domain can be a tissue factor mutant, wherein a wildtype tissue factor polypeptide lacking the signal sequence, the transmembrane domain, and the intracellular domain, and has been further modified at selected amino acids. In some examples, the soluble tissue factor domain can be a soluble human tissue factor domain. In some examples, the soluble tissue factor domain can be a soluble mouse tissue factor domain. In some examples, the soluble tissue factor domain can be a soluble rat tissue factor domain. Non-limiting examples of soluble human tissue factor domains, a mouse soluble tissue factor domain, a rat soluble tissue factor domain, and mutant soluble tissue factor domains are shown below.

Exemplary Soluble Human Tissue Factor Domain
(SEQ ID NO: 1)
SGTTNTVAAYNLTWKSTNFKTILEWEPKPVNQVYTVQISTKSGDWKSKC
FYTTDTECDLTDEIVKDVKQTYLARVFSYPAGNVESTGSAGEPLYENSP
EFTPYLETNLGQPTIQSFEQVGTKVNVTVEDERTLVRRNNTFLSLRDVF
GKDLIYTLYYWKSSSSGKKTAKTNTNEFLIDVDKGENYCFSVQAVIPSR
TVNRKSTDSPVECMGQEKGEFRE
Exemplary Nucleic Acid Encoding Soluble Human
Tissue Factor Domain
(SEQ ID NO: 2)
AGCGGCACAACCAACACAGTCGCTGCCTATAACCTCACTTGGAAGAGCA
CCAACTTCAAAACCATCCTCGAATGGGAACCCAAACCCGTTAACCAAGT
TTACACCGTGCAGATCAGCACCAAGTCCGGCGACTGGAAGTCCAAATGT
TTCTATACCACCGACACCGAGTGCGATCTCACCGATGAGATCGTGAAAG
ATGTGAAACAGACCTACCTCGCCCGGGTGTTTAGCTACCCCGCCGGCAA
TGTGGAGAGCACTGGTTCCGCTGGCGAGCCTTTATACGAGAACAGCCCC
GAATTTACCCCTTACCTCGAGACCAATTTAGGACAGCCCACCATCCAAA
GCTTTGAGCAAGTTGGCACAAAGGTGAATGTGACAGTGGAGGACGAGCG
GACTTTAGTGCGGCGGAACAACACCTTTCTCAGCCTCCGGGATGTGTTC
GGCAAAGATTTAATCTACACACTGTATTACTGGAAGTCCTCTTCCTCCG
GCAAGAAGACAGCTAAAACCAACACAAACGAGTTTTTAATCGACGTGGA
TAAAGGCGAAAACTACTGTTTCAGCGTGCAAGCTGTGATCCCCTCCCGG
ACCGTGAATAGGAAAAGCACCGATAGCCCCGTTGAGTGCATGGGCCAAG
AAAAGGGCGAGTTCCGGGAG
Exemplary Mutant Soluble Human Tissue Factor
Domain
(SEQ ID NO: 3)
SGTTNTVAAYNLTWKSTNFATALEWEPKPVNQVYTVQISTKSGDWKSKC
FYTTDTECALTDEIVKDVKQTYLARVFSYPAGNVESTGSAGEPLYENSP
EFTPYLETNLGQPTIQSFEQVGTKVNVTVEDERTLVARNNTALSLRDVF
GKDLIYTLYYWKSSSSGKKTAKTNTNEFLIDVDKGENYCFSVQAVIPSR
TVNRKSTDSPVECMGQEKGEFRE
Exemplary Mutant Soluble Human Tissue Factor
Domain
(SEQ ID NO: 4)
SGTTNTVAAYNLTWKSTNFATALEWEPKPVNQVYTVQISTKSGDAKSKC
FYTTDTECALTDEIVKDVKQTYLARVFSYPAGNVESTGSAGEPLAENSP
EFTPYLETNLGQPTIQSFEQVGTKVNVTVEDERTLVARNNTALSLRDVF
GKDLIYTLYYWKSSSSGKKTAKTNTNEFLIDVDKGENYCFSVQAVIPSR
TVNRKSTDSPVECMGQEKGEFRE
Exemplary Soluble Mouse Tissue Factor Domain
(SEQ ID NO: 5)
agipekafnltwistdfktilewqpkptnytytvqisdrsrnwknkcfs
ttdtecdltdeivkdvtwayeakvlsvprrnsvhgdgdqlvihgeeppf
tnapkflpyrdtnlgqpviqqfeqdgrklnvvvkdsltlvrkngtfltl
rqvfgkdlgyiityrkgsstgkktnitntnefsidveegvsycffvqam
ifsrktnqnspgsstvcteqwksflge
Exemplary Soluble Rat Tissue Factor Domain
(SEQ ID NO: 6)
Agtppgkafnltwistdfktilewqpkptnytytvqisdrsrnwkykct
gttdtecdltdeivkdvnwtyearvlsvpwinsthgketlfgthgeepp
ftnarkflpyrdtkigqpviqkyeqggtklkvtvkdsftlvrkngtflt
lrqvfgndlgyiltyrkdsstgrktntthtneflidvekgvsycffaqa
vifsrktnhkspesitkcteqwksvlge

In some embodiments, a soluble tissue factor domain can include a sequence that is at least 70% identical, at least 72% identical, at least 74% identical, at least 76% identical, at least 78% identical, at least 80% identical, at least 82% identical, at least 84% identical, at least 86% identical, at least 88% identical, at least 90% identical, at least 92% identical, at least 94% identical, at least 96% identical, at least 98% identical, at least 99% identical, or 100% identical to SEQ ID NO: 1, 3, 4, 5, or 6. In some embodiments, a soluble tissue factor domain can include a sequence of SEQ ID NO: 1, 3, 4, 5, or 6, with one to twenty amino acids (e.g., 2, 3, 4, 5, 6, 7, 8, 9, 10, 11, 12, 13, 14, 15, 16, 17, 18, 19, or 20) amino acids removed from its N-terminus and/or one to twenty amino acids (e.g., 2, 3, 4, 5, 6, 7, 8, 9, 10, 11, 12, 13, 14, 15, 16, 17, 18, 19, or 20) amino acids removed from its C-terminus.

As can be appreciated in the art, one skilled in the art would understand that mutation of amino acids that are conserved between different mammalian species is more likely to decrease the activity and/or structural stability of the protein, while mutation of amino acids that are not conserved between different mammalian species is less likely to decrease the activity and/or structural stability of the protein.

In some examples of any of the multi-chain chimeric polypeptides described herein, the soluble tissue factor domain is not capable of binding to Factor VIIa. In some examples of any of the multi-chain chimeric polypeptides described herein, the soluble tissue factor domain does not convert inactive Factor X into Factor Xa. In some embodiments of any of the multi-chain chimeric polypeptides described herein, the multi-chain chimeric polypeptide does not stimulate blood coagulation in a mammal. In some embodiments of any of the single-chain chimeric polypeptides provided herein, the human soluble tissue factor domain does not initiate blood coagulation.

In some examples, the soluble tissue factor domain can be a soluble human tissue factor domain. In some embodiments, the soluble tissue factor domain can be a soluble mouse tissue factor domain. In some embodiments, the soluble tissue factor domain can be a soluble rat tissue factor domain.

In some examples, the soluble tissue factor domain does not include one or more (e.g., two, three, four, five, six, or seven) of: a lysine at an amino acid position that corresponds to amino acid position 20 of mature wildtype human tissue factor protein; an isoleucine at an amino acid position that corresponds to amino acid position 22 of mature wildtype human tissue factor protein; a tryptophan at an amino acid position that corresponds to amino acid position 45 of mature wildtype human tissue factor protein; an aspartic acid at an amino acid position that corresponds to amino acid position 58 of mature wildtype human tissue factor protein; a tyrosine at an amino acid position that corresponds to amino acid position 94 of mature wildtype human tissue factor protein; an arginine at an amino acid position that corresponds to amino acid position 135 of mature wildtype human tissue factor protein; and a phenylalanine at an amino acid position that corresponds to amino acid position 140 of mature wildtype human tissue factor protein. In some embodiments, the mutant soluble tissue factor possesses the amino acid sequence of SEQ ID NO: 3 or SEQ ID NO: 4.

In some examples, the soluble tissue factor domain can be encoded by a nucleic acid including a sequence that is at least 70% identical, at least 72% identical, at least 74% identical, at least 76% identical, at least 78% identical, at least 80% identical, at least 82% identical, at least 84% identical, at least 86% identical, at least 88% identical, at least 90% identical, at least 92% identical, at least 94% identical, at least 96% identical, at least 98% identical, at least 99% identical, or 100% identical to SEQ ID NO: 2.

In some embodiments, the soluble tissue factor domain can have a total length of about 20 amino acids to about 220 amino acids, about 20 amino acids to about 215 amino acids, about 20 amino acids to about 210 amino acids, about 20 amino acids to about 205 amino acids, about 20 amino acids to about 200 amino acids, about 20 amino acids to about 195 amino acids, about 20 amino acids to about 190 amino acids, about 20 amino acids to about 185 amino acids, about 20 amino acids to about 180 amino acids, about 20 amino acids to about 160 amino acids, about 20 amino acids to about 140 amino acids, about 20 amino acids to about 120 amino acids, about 20 amino acids to about 100 amino acids, about 20 amino acids to about 80 amino acids, about 20 amino acids to about 60 amino acids, about 20 amino acids to about 40 amino acids, about 40 amino acids to about 220 amino acids, about 40 amino acids to about 215 amino acids, about 40 amino acids to about 210 amino acids, about 40 amino acids to about 205 amino acids, about 40 amino acids to about 200 amino acids, about 40 amino acids to about 195 amino acids, about 40 amino acids to about 190 amino acids, about 40 amino acids to about 185 amino acids, about 40 amino acids to about 180 amino acids, about 40 amino acids to about 160 amino acids, about 40 amino acids to about 140 amino acids, about 40 amino acids to about 120 amino acids, about 40 amino acids to about 100 amino acids, about 40 amino acids to about 80 amino acids, about 40 amino acids to about 60 amino acids, about 60 amino acids to about 220 amino acids, about 60 amino acids to about 215 amino acids, about 60 amino acids to about 210 amino acids, about 60 amino acids to about 205 amino acids, about 60 amino acids to about 200 amino acids, about 60 amino acids to about 195 amino acids, about 60 amino acids to about 190 amino acids, about 60 amino acids to about 185 amino acids, about 60 amino acids to about 180 amino acids, about 60 amino acids to about 160 amino acids, about 60 amino acids to about 140 amino acids, about 60 amino acids to about 120 amino acids, about 60 amino acids to about 100 amino acids, about 60 amino acids to about 80 amino acids, about 80 amino acids to about 220 amino acids, about 80 amino acids to about 215 amino acids, about 80 amino acids to about 210 amino acids, about 80 amino acids to about 205 amino acids, about 80 amino acids to about 200 amino acids, about 80 amino acids to about 195 amino acids, about 80 amino acids to about 190 amino acids, about 80 amino acids to about 185 amino acids, about 80 amino acids to about 180 amino acids, about 80 amino acids to about 160 amino acids, about 80 amino acids to about 140 amino acids, about 80 amino acids to about 120 amino acids, about 80 amino acids to about 100 amino acids, about 100 amino acids to about 220 amino acids, about 100 amino acids to about 215 amino acids, about 100 amino acids to about 210 amino acids, about 100 amino acids to about 205 amino acids, about 100 amino acids to about 200 amino acids, about 100 amino acids to about 195 amino acids, about 100 amino acids to about 190 amino acids, about 100 amino acids to about 185 amino acids, about 100 amino acids to about 180 amino acids, about 100 amino acids to about 160 amino acids, about 100 amino acids to about 140 amino acids, about 100 amino acids to about 120 amino acids, about 120 amino acids to about 220 amino acids, about 120 amino acids to about 215 amino acids, about 120 amino acids to about 210 amino acids, about 120 amino acids to about 205 amino acids, about 120 amino acids to about 200 amino acids, about 120 amino acids to about 195 amino acids, about 120 amino acids to about 190 amino acids, about 120 amino acids to about 185 amino acids, about 120 amino acids to about 180 amino acids, about 120 amino acids to about 160 amino acids, about 120 amino acids to about 140 amino acids, about 140 amino acids to about 220 amino acids, about 140 amino acids to about 215 amino acids, about 140 amino acids to about 210 amino acids, about 140 amino acids to about 205 amino acids, about 140 amino acids to about 200 amino acids, about 140 amino acids to about 195 amino acids, about 140 amino acids to about 190 amino acids, about 140 amino acids to about 185 amino acids, about 140 amino acids to about 180 amino acids, about 140 amino acids to about 160 amino acids, about 160 amino acids to about 220 amino acids, about 160 amino acids to about 215 amino acids, about 160 amino acids to about 210 amino acids, about 160 amino acids to about 205 amino acids, about 160 amino acids to about 200 amino acids, about 160 amino acids to about 195 amino acids, about 160 amino acids to about 190 amino acids, about 160 amino acids to about 185 amino acids, about 160 amino acids to about 180 amino acids, about 180 amino acids to about 220 amino acids, about 180 amino acids to about 215 amino acids, about 180 amino acids to about 210 amino acids, about 180 amino acids to about 205 amino acids, about 180 amino acids to about 200 amino acids, about 180 amino acids to about 195 amino acids, about 180 amino acids to about 190 amino acids, about 180 amino acids to about 185 amino acids, about 185 amino acids to about 220 amino acids, about 185 amino acids to about 215 amino acids, about 185 amino acids to about 210 amino acids, about 185 amino acids to about 205 amino acids, about 185 amino acids to about 200 amino acids, about 185 amino acids to about 195 amino acids, about 185 amino acids to about 190 amino acids, about 190 amino acids to about 220 amino acids, about 190 amino acids to about 215 amino acids, about 190 amino acids to about 210 amino acids, about 190 amino acids to about 205 amino acids, about 190 amino acids to about 200 amino acids, about 190 amino acids to about 195 amino acids, about 195 amino acids to about 220 amino acids, about 195 amino acids to about 215 amino acids, about 195 amino acids to about 210 amino acids, about 195 amino acids to about 205 amino acids, about 195 amino acids to about 200 amino acids, about 200 amino acids to about 220 amino acids, about 200 amino acids to about 215 amino acids, about 200 amino acids to about 210 amino acids, about 200 amino acids to about 205 amino acids, about 205 amino acids to about 220 amino acids, about 205 amino acids to about 215 amino acids, about 205 amino acids to about 210 amino acids, about 210 amino acids to about 220 amino acids, about 210 amino acids to about 215 amino acids, or about 215 amino acids to about 220 amino acids.

### Linker Sequences

In some embodiments, the linker sequence can be a flexible linker sequence. Non-limiting examples of linker sequences that can be used are described in Klein et al., Protein Engineering, Design & Selection 27 (10): 325-330, 2014; Priyanka et al., Protein Sci. 22 (2): 153-167, 2013. In some examples, the linker sequence is a synthetic linker sequence.

In some embodiments of any of the multi-chain chimeric polypeptides described herein, the first chimeric polypeptide can include one, two, three, four, five, six, seven, eight, nine, or ten linker sequence(s) (e.g., the same or different linker sequences, e.g., any of the exemplary linker sequences described herein or known in the art). In some embodiments of any of the multi-chain chimeric polypeptides described herein, the second chimeric polypeptide can include one, two, three, four, five, six, seven, eight, nine, or ten linker sequence(s) (e.g., the same or different linker sequences, e.g., any of the exemplary linker sequences described herein or known in the art).

In some embodiments, a linker sequence can have a total length of 1 amino acid to about 100 amino acids, 1 amino acid to about 90 amino acids, 1 amino acid to about 80 amino acids, 1 amino acid to about 70 amino acids, 1 amino acid to about 60 amino acids, 1 amino acid to about 50 amino acids, 1 amino acid to about 40 amino acids, 1 amino acid to about 30 amino acids, 1 amino acid to about 25 amino acids, 1 amino acid to about 20 amino acids, 1 amino acid to about 15 amino acids, 1 amino acid to about 10 amino acids, 1 amino acid to about 8 amino acids, 1 amino acid to about 6 amino acids, 1 amino acid to about 4 amino acids, about 2 amino acids to about 100 amino acids, about 2 amino acids to about 90 amino acids, about 2 amino acids to about 80 amino acids, about 2 amino acids to about 70 amino acids, about 2 amino acids to about 60 amino acids, about 2 amino acids to about 50 amino acids, about 2 amino acids to about 40 amino acids, about 2 amino acids to about 30 amino acids, about 2 amino acids to about 25 amino acids, about 2 amino acids to about 20 amino acids, about 2 amino acids to about 15 amino acids, about 2 amino acids to about 10 amino acids, about 2 amino acids to about 8 amino acids, about 2 amino acids to about 6 amino acids, about 2 amino acids to about 4 amino acids, about 4 amino acids to about 100 amino acids, about 4 amino acids to about 90 amino acids, about 4 amino acids to about 80 amino acids, about 4 amino acids to about 70 amino acids, about 4 amino acids to about 60 amino acids, about 4 amino acids to about 50 amino acids, about 4 amino acids to about 40 amino acids, about 4 amino acids to about 30 amino acids, about 4 amino acids to about 25 amino acids, about 4 amino acids to about 20 amino acids, about 4 amino acids to about 15 amino acids, about 4 amino acids to about 10 amino acids, about 4 amino acids to about 8 amino acids, about 4 amino acids to about 6 amino acids, about 6 amino acids to about 100 amino acids, about 6 amino acids to about 90 amino acids, about 6 amino acids to about 80 amino acids, about 6 amino acids to about 70 amino acids, about 6 amino acids to about 60 amino acids, about 6 amino acids to about 50 amino acids, about 6 amino acids to about 40 amino acids, about 6 amino acids to about 30 amino acids, about 6 amino acids to about 25 amino acids, about 6 amino acids to about 20 amino acids, about 6 amino acids to about 15 amino acids, about 6 amino acids to about 10 amino acids, about 6 amino acids to about 8 amino acids, about 8 amino acids to about 100 amino acids, about 8 amino acids to about 90 amino acids, about 8 amino acids to about 80 amino acids, about 8 amino acids to about 70 amino acids, about 8 amino acids to about 60 amino acids, about 8 amino acids to about 50 amino acids, about 8 amino acids to about 40 amino acids, about 8 amino acids to about 30 amino acids, about 8 amino acids to about 25 amino acids, about 8 amino acids to about 20 amino acids, about 8 amino acids to about 15 amino acids, about 8 amino acids to about 10 amino acids, about 10 amino acids to about 100 amino acids, about 10 amino acids to about 90 amino acids, about 10 amino acids to about 80 amino acids, about 10 amino acids to about 70 amino acids, about 10 amino acids to about 60 amino acids, about 10 amino acids to about 50 amino acids, about 10 amino acids to about 40 amino acids, about 10 amino acids to about 30 amino acids, about 10 amino acids to about 25 amino acids, about 10 amino acids to about 20 amino acids, about 10 amino acids to about 15 amino acids, about 15 amino acids to about 100 amino acids, about 15 amino acids to about 90 amino acids, about 15 amino acids to about 80 amino acids, about 15 amino acids to about 70 amino acids, about 15 amino acids to about 60 amino acids, about 15 amino acids to about 50 amino acids, about 15 amino acids to about 40 amino acids, about 15 amino acids to about 30 amino acids, about 15 amino acids to about 25 amino acids, about 15 amino acids to about 20 amino acids, about 20 amino acids to about 100 amino acids, about 20 amino acids to about 90 amino acids, about 20 amino acids to about 80 amino acids, about 20 amino acids to about 70 amino acids, about 20 amino acids to about 60 amino acids, about 20 amino acids to about 50 amino acids, about 20 amino acids to about 40 amino acids, about 20 amino acids to about 30 amino acids, about 20 amino acids to about 25 amino acids, about 25 amino acids to about 100 amino acids, about 25 amino acids to about 90 amino acids, about 25 amino acids to about 80 amino acids, about 25 amino acids to about 70 amino acids, about 25 amino acids to about 60 amino acids, about 25 amino acids to about 50 amino acids, about 25 amino acids to about 40 amino acids, about 25 amino acids to about 30 amino acids, about 30 amino acids to about 100 amino acids, about 30 amino acids to about 90 amino acids, about 30 amino acids to about 80 amino acids, about 30 amino acids to about 70 amino acids, about 30 amino acids to about 60 amino acids, about 30 amino acids to about 50 amino acids, about 30 amino acids to about 40 amino acids, about 40 amino acids to about 100 amino acids, about 40 amino acids to about 90 amino acids, about 40 amino acids to about 80 amino acids, about 40 amino acids to about 70 amino acids, about 40 amino acids to about 60 amino acids, about 40 amino acids to about 50 amino acids, about 50 amino acids to about 100 amino acids, about 50 amino acids to about 90 amino acids, about 50 amino acids to about 80 amino acids, about 50 amino acids to about 70 amino acids, about 50 amino acids to about 60 amino acids, about 60 amino acids to about 100 amino acids, about 60 amino acids to about 90 amino acids, about 60 amino acids to about 80 amino acids, about 60 amino acids to about 70 amino acids, about 70 amino acids to about 100 amino acids, about 70 amino acids to about 90 amino acids, about 70 amino acids to about 80 amino acids, about 80 amino acids to about 100 amino acids, about 80 amino acids to about 90 amino acids, or about 90 amino acids to about 100 amino acids.

In some embodiments, the linker is rich in glycine (Gly or G) residues. In some embodiments, the linker is rich in serine (Ser or S) residues. In some embodiments, the linker is rich in glycine and serine residues. In some embodiments, the linker has one or more glycine-serine residue pairs (GS), e.g., 1, 2, 3, 4, 5, 6, 7, 8, 9, or 10 or more GS pairs. In some embodiments, the linker has one or more Gly-Gly-Gly-Ser (GGGS) sequences, e.g., 1, 2, 3, 4, 5, 6, 7, 8, 9, or 10 or more GGGS sequences. In some embodiments, the linker has one or more Gly-Gly-Gly-Gly-Ser (GGGGS) sequences, e.g., 1, 2, 3, 4, 5, 6, 7, 8, 9, or 10 or more GGGGS sequences. In some embodiments, the linker has one or more Gly-Gly-Ser-Gly (GGSG) sequences, e.g., 1, 2, 3, 4, 5, 6, 7, 8, 9, or 10 or more GGSG sequences.

In some embodiments, the linker sequence can comprise or consist of GGGGSGGGGSGGGGS (SEQ ID NO: 7). In some embodiments, the linker sequence can be encoded by a nucleic acid comprising or consisting of: GGCGGTGGAGGATCCGGAGGAGGTGGCTCCGGCGGCGGAGGATCT (SEQ ID NO: 8). In some embodiments, the linker sequence can comprise or consist of: GGGSGGGS (SEQ ID NO: 9).

### Target-Binding Domains

In some embodiments of any of the multi-chain chimeric polypeptides described herein, the first target-binding domain, the second target-binding domain, and/or the additional one or more target-binding domains can be an antigen-binding domain (e.g., any of the exemplary antigen-binding domains described herein or known in the art), a soluble interleukin or cytokine protein (e.g., any of the exemplary soluble interleukin proteins or soluble cytokine proteins described herein), and a soluble interleukin or cytokine receptor (e.g., any of the exemplary soluble interleukin receptors or soluble cytokine receptors described herein).

In some embodiments of any of the multi-chain chimeric polypeptides described herein, one or both of the first target-binding domain and the second target-binding domain is an antigen-binding domain. In some embodiments of any of the multi-chain chimeric polypeptides described herein, one or both of the first target-binding domain and the second target-binding domain is a soluble interleukin or cytokine receptor.

In some embodiments of any of the multi-chain chimeric polypeptides described herein, the first chimeric polypeptide further comprises one or more additional target-binding domain(s). In some embodiments of any of the multi-chain chimeric polypeptides described herein, the second chimeric polypeptide further comprises one or more additional target-binding domain(s).

In some embodiments of any of the multi-chain chimeric polypeptides described herein, the one or more additional target binding domains can each, independently, bind specifically to a target selected from the group of: bind specifically to a target selected from the group consisting of: CD16a, CD28, CD3 (e.g., one or more of CD3α, CD3β, CD3δ, CD3ε, and CD3γ), CD33, CD20, CD19, CD22, CD123, IL-1R, IL-1, VEGF, IL-6R, IL-4, IL-10, PDL-1, TIGIT, PD-1, TIM3, CTLA4, MICA, MICB, IL-6, IL-8, TNFα, CD26a, CD36, ULBP2, CD30, CD200, IGF-1R, MUC4AC, MUC5AC, Trop-2, CMET, EGFR, HER1, HER2, HER3, PSMA, CEA, B7H3, EPCAM, BCMA, P-cadherin, CEACAM5, a UL16-binding protein (e.g., ULBP1, ULBP2, ULBP3, ULBP4, ULBP5, and ULBP6), HLA-DR, DLL4, TYRO3, AXL, MER, CD122, CD155, PDGF-DD, a ligand of TGF-β receptor II (TGF-β RII), a ligand of TGF-β RIII, a ligand of DNAM-1, a ligand of NKD46, a ligand of NKD44, a ligand of NKG2D, a ligand of NKD30, a ligand for a scMHCI, a ligand for a scMHCII, a ligand for a scTCR, a receptor for IL-1, a receptor for IL-2, a receptor for IL-3, a receptor for IL-7, a receptor for IL-8, a receptor for IL-10, a receptor for IL-12, a receptor for IL-15, a receptor for IL-17, a receptor for IL-18, a receptor for IL-21, a receptor for PDGF-DD, a receptor for stem cell factor (SCF), a receptor for stem cell-like tyrosine kinase 3 ligand (FLT3L), a receptor for MICA, a receptor for MICB, a receptor for a ULP16-binding protein, a receptor for CD155, a receptor for CD122, and a receptor for CD28.

In some embodiments of any of the multi-chain chimeric polypeptides described herein, the first target-binding domain, the second target-binding domain, and/or the one or more additional target-binding domains can each independent have a total number of amino acids of about 5 amino acids to about 1000 amino acids, about 5 amino acids to about 900 amino acids, about 5 amino acids to about 800 amino acids, about 5 amino acids to about 700 amino acids, about 5 amino acids to about 600 amino acids, about 5 amino acids to about 500 amino acids, about 5 amino acids to about 400 amino acids, about 5 amino acids to about 300 amino acids, about 5 amino acids to about 280 amino acids, about 5 amino acids to about 260 amino acids, about 5 amino acids to about 240 amino acids, about 5 amino acids to about 220 amino acids, about 5 amino acids to about 200 amino acids, about 5 amino acids to about 180 amino acids, about 5 amino acids to about 160 amino acids, about 5 amino acids to about 140 amino acids, about 5 amino acids to about 120 amino acids, about 5 amino acids to about 100 amino acids, about 5 amino acids to about 80 amino acids, about 5 amino acids to about 60 amino acids, about 5 amino acids to about 40 amino acids, about 5 amino acids to about 20 amino acids, about 5 amino acids to about 10 amino acids, about 10 amino acids to about 1000 amino acids, about 10 amino acids to about 900 amino acids, about 10 amino acids to about 800 amino acids, about 10 amino acids to about 700 amino acids, about 10 amino acids to about 600 amino acids, about 10 amino acids to about 500 amino acids, about 10 amino acids to about 400 amino acids, about 10 amino acids to about 300 amino acids, about 10 amino acids to about 280 amino acids, about 10 amino acids to about 260 amino acids, about 10 amino acids to about 240 amino acids, about 10 amino acids to about 220 amino acids, about 10 amino acids to about 200 amino acids, about 10 amino acids to about 180 amino acids, about 10 amino acids to about 160 amino acids, about 10 amino acids to about 140 amino acids, about 10 amino acids to about 120 amino acids, about 10 amino acids to about 100 amino acids, about 10 amino acids to about 80 amino acids, about 10 amino acids to about 60 amino acids, about 10 amino acids to about 40 amino acids, about 10 amino acids to about 20 amino acids, about 20 amino acids to about 1000 amino acids, about 20 amino acids to about 900 amino acids, about 20 amino acids to about 800 amino acids, about 20 amino acids to about 700 amino acids, about 20 amino acids to about 600 amino acids, about 20 amino acids to about 500 amino acids, about 20 amino acids to about 400 amino acids, about 20 amino acids to about 300 amino acids, about 20 amino acids to about 280 amino acids, about 20 amino acids to about 260 amino acids, about 20 amino acids to about 240 amino acids, about 20 amino acids to about 220 amino acids, about 20 amino acids to about 200 amino acids, about 20 amino acids to about 180 amino acids, about 20 amino acids to about 160 amino acids, about 20 amino acids to about 140 amino acids, about 20 amino acids to about 120 amino acids, about 20 amino acids to about 100 amino acids, about 20 amino acids to about 80 amino acids, about 20 amino acids to about 60 amino acids, about 20 amino acids to about 40 amino acids, about 40 amino acids to about 1000 amino acids, about 40 amino acids to about 900 amino acids, about 40 amino acids to about 800 amino acids, about 40 amino acids to about 700 amino acids, about 40 amino acids to about 600 amino acids, about 40 amino acids to about 500 amino acids, about 40 amino acids to about 400 amino acids, about 40 amino acids to about 300 amino acids, about 40 amino acids to about 280 amino acids, about 40 amino acids to about 260 amino acids, about 40 amino acids to about 240 amino acids, about 40 amino acids to about 220 amino acids, about 40 amino acids to about 200 amino acids, about 40 amino acids to about 180 amino acids, about 40 amino acids to about 160 amino acids, about 40 amino acids to about 140 amino acids, about 40 amino acids to about 120 amino acids, about 40 amino acids to about 100 amino acids, about 40 amino acids to about 80 amino acids, about 40 amino acids to about 60 amino acids, about 60 amino acids to about 1000 amino acids, about 60 amino acids to about 900 amino acids, about 60 amino acids to about 800 amino acids, about 60 amino acids to about 700 amino acids, about 60 amino acids to about 600 amino acids, about 60 amino acids to about 500 amino acids, about 60 amino acids to about 400 amino acids, about 60 amino acids to about 300 amino acids, about 60 amino acids to about 280 amino acids, about 60 amino acids to about 260 amino acids, about 60 amino acids to about 240 amino acids, about 60 amino acids to about 220 amino acids, about 60 amino acids to about 200 amino acids, about 60 amino acids to about 180 amino acids, about 60 amino acids to about 160 amino acids, about 60 amino acids to about 140 amino acids, about 60 amino acids to about 120 amino acids, about 60 amino acids to about 100 amino acids, about 60 amino acids to about 80 amino acids, about 80 amino acids to about 1000 amino acids, about 80 amino acids to about 900 amino acids, about 80 amino acids to about 800 amino acids, about 80 amino acids to about 700 amino acids, about 80 amino acids to about 600 amino acids, about 80 amino acids to about 500 amino acids, about 80 amino acids to about 400 amino acids, about 80 amino acids to about 300 amino acids, about 80 amino acids to about 280 amino acids, about 80 amino acids to about 260 amino acids, about 80 amino acids to about 240 amino acids, about 80 amino acids to about 220 amino acids, about 80 amino acids to about 200 amino acids, about 80 amino acids to about 180 amino acids, about 80 amino acids to about 160 amino acids, about 80 amino acids to about 140 amino acids, about 80 amino acids to about 120 amino acids, about 80 amino acids to about 100 amino acids, about 100 amino acids to about 1000 amino acids, about 100 amino acids to about 900 amino acids, about 100 amino acids to about 800 amino acids, about 100 amino acids to about 700 amino acids, about 100 amino acids to about 600 amino acids, about 100 amino acids to about 500 amino acids, about 100 amino acids to about 400 amino acids, about 100 amino acids to about 300 amino acids, about 100 amino acids to about 280 amino acids, about 100 amino acids to about 260 amino acids, about 100 amino acids to about 240 amino acids, about 100 amino acids to about 220 amino acids, about 100 amino acids to about 200 amino acids, about 100 amino acids to about 180 amino acids, about 100 amino acids to about 160 amino acids, about 100 amino acids to about 140 amino acids, about 100 amino acids to about 120 amino acids, about 120 amino acids to about 1000 amino acids, about 120 amino acids to about 900 amino acids, about 120 amino acids to about 800 amino acids, about 120 amino acids to about 700 amino acids, about 120 amino acids to about 600 amino acids, about 120 amino acids to about 500 amino acids, about 120 amino acids to about 400 amino acids, about 120 amino acids to about 300 amino acids, about 120 amino acids to about 280 amino acids, about 120 amino acids to about 260 amino acids, about 120 amino acids to about 240 amino acids, about 120 amino acids to about 220 amino acids, about 120 amino acids to about 200 amino acids, about 120 amino acids to about 180 amino acids, about 120 amino acids to about 160 amino acids, about 120 amino acids to about 140 amino acids, about 140 amino acids to about 1000 amino acids, about 140 amino acids to about 900 amino acids, about 140 amino acids to about 800 amino acids, about 140 amino acids to about 700 amino acids, about 140 amino acids to about 600 amino acids, about 140 amino acids to about 500 amino acids, about 140 amino acids to about 400 amino acids, about 140 amino acids to about 300 amino acids, about 140 amino acids to about 280 amino acids, about 140 amino acids to about 260 amino acids, about 140 amino acids to about 240 amino acids, about 140 amino acids to about 220 amino acids, about 140 amino acids to about 200 amino acids, about 140 amino acids to about 180 amino acids, about 140 amino acids to about 160 amino acids, about 160 amino acids to about 1000 amino acids, about 160 amino acids to about 900 amino acids, about 160 amino acids to about 800 amino acids, about 160 amino acids to about 700 amino acids, about 160 amino acids to about 600 amino acids, about 160 amino acids to about 500 amino acids, about 160 amino acids to about 400 amino acids, about 160 amino acids to about 300 amino acids, about 160 amino acids to about 280 amino acids, about 160 amino acids to about 260 amino acids, about 160 amino acids to about 240 amino acids, about 160 amino acids to about 220 amino acids, about 160 amino acids to about 200 amino acids, about 160 amino acids to about 180 amino acids, about 180 amino acids to about 1000 amino acids, about 180 amino acids to about 900 amino acids, about 180 amino acids to about 800 amino acids, about 180 amino acids to about 700 amino acids, about 180 amino acids to about 600 amino acids, about 180 amino acids to about 500 amino acids, about 180 amino acids to about 400 amino acids, about 180 amino acids to about 300 amino acids, about 180 amino acids to about 280 amino acids, about 180 amino acids to about 260 amino acids, about 180 amino acids to about 240 amino acids, about 180 amino acids to about 220 amino acids, about 180 amino acids to about 200 amino acids, about 200 amino acids to about 1000 amino acids, about 200 amino acids to about 900 amino acids, about 200 amino acids to about 800 amino acids, about 200 amino acids to about 700 amino acids, about 200 amino acids to about 600 amino acids, about 200 amino acids to about 500 amino acids, about 200 amino acids to about 400 amino acids, about 200 amino acids to about 300 amino acids, about 200 amino acids to about 280 amino acids, about 200 amino acids to about 260 amino acids, about 200 amino acids to about 240 amino acids, about 200 amino acids to about 220 amino acids, about 220 amino acids to about 1000 amino acids, about 220 amino acids to about 900 amino acids, about 220 amino acids to about 800 amino acids, about 220 amino acids to about 700 amino acids, about 220 amino acids to about 600 amino acids, about 220 amino acids to about 500 amino acids, about 220 amino acids to about 400 amino acids, about 220 amino acids to about 300 amino acids, about 220 amino acids to about 280 amino acids, about 220 amino acids to about 260 amino acids, about 220 amino acids to about 240 amino acids, about 240 amino acids to about 1000 amino acids, about 240 amino acids to about 900 amino acids, about 240 amino acids to about 800 amino acids, about 240 amino acids to about 700 amino acids, about 240 amino acids to about 600 amino acids, about 240 amino acids to about 500 amino acids, about 240 amino acids to about 400 amino acids, about 240 amino acids to about 300 amino acids, about 240 amino acids to about 280 amino acids, about 240 amino acids to about 260 amino acids, about 260 amino acids to about 1000 amino acids, about 260 amino acids to about 900 amino acids, about 260 amino acids to about 800 amino acids, about 260 amino acids to about 700 amino acids, about 260 amino acids to about 600 amino acids, about 260 amino acids to about 500 amino acids, about 260 amino acids to about 400 amino acids, about 260 amino acids to about 300 amino acids, about 260 amino acids to about 280 amino acids, about 280 amino acids to about 1000 amino acids, about 280 amino acids to about 900 amino acids, about 280 amino acids to about 800 amino acids, about 280 amino acids to about 700 amino acids, about 280 amino acids to about 600 amino acids, about 280 amino acids to about 500 amino acids, about 280 amino acids to about 400 amino acids, about 280 amino acids to about 300 amino acids, about 300 amino acids to about 1000 amino acids, about 300 amino acids to about 900 amino acids, about 300 amino acids to about 800 amino acids, about 300 amino acids to about 700 amino acids, about 300 amino acids to about 600 amino acids, about 300 amino acids to about 500 amino acids, about 300 amino acids to about 400 amino acids, about 400 amino acids to about 1000 amino acids, about 400 amino acids to about 900 amino acids, about 400 amino acids to about 800 amino acids, about 400 amino acids to about 700 amino acids, about 400 amino acids to about 600 amino acids, about 400 amino acids to about 500 amino acids, about 500 amino acids to about 1000 amino acids, about 500 amino acids to about 900 amino acids, about 500 amino acids to about 800 amino acids, about 500 amino acids to about 700 amino acids, about 500 amino acids to about 600 amino acids, about 600 amino acids to about 1000 amino acids, about 600 amino acids to about 900 amino acids, about 600 amino acids to about 800 amino acids, about 600 amino acids to about 700 amino acids, about 700 amino acids to about 1000 amino acids, about 700 amino acids to about 900 amino acids, about 700 amino acids to about 800 amino acids, about 800 amino acids to about 1000 amino acids, about 800 amino acids to about 900 amino acids, or about 900 amino acids to about 1000 amino acids.

Any of the target-binding domains described herein can bind to its target with a dissociation equilibrium constant (K_{D}) of less than 1×10⁻⁷M, less than 1×10⁻⁸ M, less than 1×10⁻⁹M, less than 1×10⁻¹⁰ M, less than 1×10⁻¹¹ M, less than 1×10⁻¹² M, or less than 1×10⁻¹³ M. In some embodiments, the antigen-binding protein construct provided herein can bind to an identifying antigen with a K_{D} of about 1×10⁻³ M to about 1×10⁻⁵ M, about 1×10⁻⁴ M to about 1×10⁻⁶ M, about 1×10⁻⁵M to about 1×10⁻⁷ M, about 1×10⁻⁶ M to about 1×10⁻⁸ M, about 1×10⁻⁷ M to about 1×10⁻⁹M, about 1×10⁻⁸ M to about 1×10⁻¹⁰ M, or about 1×10⁻⁹ M to about 1×10⁻¹¹ M (inclusive).

Any of the target-binding domains described herein can bind to its target with a K_{D} of between about 1 pM to about 30 nM (e.g., about 1 pM to about 25 nM, about 1 pM to about 20 nM, about 1 pM to about 15 nM, about 1 pM to about 10 nM, about 1 pM to about 5 nM, about 1 pM to about 2 nM, about 1 pM to about 1 nM, about 1 pM to about 950 pM, about 1 pM to about 900 pM, about 1 pM to about 850 pM, about 1 pM to about 800 pM, about 1 pM to about 750 pM, about 1 pM to about 700 pM, about 1 pM to about 650 pM, about 1 pM to about 600 pM, about 1 pM to about 550 pM, about 1 pM to about 500 pM, about 1 pM to about 450 pM, about 1 pM to about 400 pM, about 1 pM to about 350 pM, about 1 pM to about 300 pM, about 1 pM to about 250 pM, about 1 pM to about 200 pM, about 1 pM to about 150 pM, about 1 pM to about 100 pM, about 1 pM to about 90 pM, about 1 pM to about 80 pM, about 1 pM to about 70 pM, about 1 pM to about 60 pM, about 1 pM to about 50 pM, about 1 pM to about 40 pM, about 1 pM to about 30 pM, about 1 pM to about 20 pM, about 1 pM to about 10 pM, about 1 pM to about 5 pM, about 1 pM to about 4 pM, about 1 pM to about 3 pM, about 1 pM to about 2 pM, about 2 pM to about 30 nM, about 2 pM to about 25 nM, about 2 pM to about 20 nM, about 2 pM to about 15 nM, about 2 pM to about 10 nM, about 2 pM to about 5 nM, about 2 pM to about 2 nM, about 2 pM to about 1 nM, about 2 pM to about 950 pM, about 2 pM to about 900 pM, about 2 pM to about 850 pM, about 2 pM to about 800 pM, about 2 pM to about 750 pM, about 2 pM to about 700 pM, about 2 pM to about 650 pM, about 2 pM to about 600 pM, about 2 pM to about 550 pM, about 2 pM to about 500 pM, about 2 pM to about 450 pM, about 2 pM to about 400 pM, about 2 pM to about 350 pM, about 2 pM to about 300 pM, about 2 pM to about 250 pM, about 2 pM to about 200 pM, about 2 pM to about 150 pM, about 2 pM to about 100 pM, about 2 pM to about 90 pM, about 2 pM to about 80 pM, about 2 pM to about 70 pM, about 2 pM to about 60 pM, about 2 pM to about 50 pM, about 2 pM to about 40 pM, about 2 pM to about 30 pM, about 2 pM to about 20 pM, about 2 pM to about 10 pM, about 2 pM to about 5 pM, about 2 pM to about 4 pM, about 2 pM to about 3 pM, about 5 pM to about 30 nM, about 5 pM to about 25 nM, about 5 pM to about 20 nM, about 5 pM to about 15 nM, about 5 pM to about 10 nM, about 5 pM to about 5 nM, about 5 pM to about 2 nM, about 5 pM to about 1 nM, about 5 pM to about 950 pM, about 5 pM to about 900 pM, about 5 pM to about 850 pM, about 5 pM to about 800 pM, about 5 pM to about 750 pM, about 5 pM to about 700 pM, about 5 pM to about 650 pM, about 5 pM to about 600 pM, about 5 pM to about 550 pM, about 5 pM to about 500 pM, about 5 pM to about 450 pM, about 5 pM to about 400 pM, about 5 pM to about 350 pM, about 5 pM to about 300 pM, about 5 pM to about 250 pM, about 5 pM to about 200 pM, about 5 pM to about 150 pM, about 5 pM to about 100 pM, about 5 pM to about 90 pM, about 5 pM to about 80 pM, about 5 pM to about 70 pM, about 5 pM to about 60 pM, about 5 pM to about 50 pM, about 5 pM to about 40 pM, about 5 pM to about 30 pM, about 5 pM to about 20 pM, about 5 pM to about 10 pM, about 10 pM to about 30 nM, about 10 pM to about 25 nM, about 10 pM to about 20 nM, about 10 pM to about 15 nM, about 10 pM to about 10 nM, about 10 pM to about 5 nM, about 10 pM to about 2 nM, about 10 pM to about 1 nM, about 10 pM to about 950 pM, about 10 pM to about 900 pM, about 10 pM to about 850 pM, about 10 pM to about 800 pM, about 10 pM to about 750 pM, about 10 pM to about 700 pM, about 10 pM to about 650 pM, about 10 pM to about 600 pM, about 10 pM to about 550 pM, about 10 pM to about 500 pM, about 10 pM to about 450 pM, about 10 pM to about 400 pM, about 10 pM to about 350 pM, about 10 pM to about 300 pM, about 10 pM to about 250 pM, about 10 pM to about 200 pM, about 10 pM to about 150 pM, about 10 pM to about 100 pM, about 10 pM to about 90 pM, about 10 pM to about 80 pM, about 10 pM to about 70 pM, about 10 pM to about 60 pM, about 10 pM to about 50 pM, about 10 pM to about 40 pM, about 10 pM to about 30 pM, about 10 pM to about 20 pM, about 15 pM to about 30 nM, about 15 pM to about 25 nM, about 15 pM to about 20 nM, about 15 pM to about 15 nM, about 15 pM to about 10 nM, about 15 pM to about 5 nM, about 15 pM to about 2 nM, about 15 pM to about 1 nM, about 15 pM to about 950 pM, about 15 pM to about 900 pM, about 15 pM to about 850 pM, about 15 pM to about 800 pM, about 15 pM to about 750 pM, about 15 pM to about 700 pM, about 15 pM to about 650 pM, about 15 pM to about 600 pM, about 15 pM to about 550 pM, about 15 pM to about 500 pM, about 15 pM to about 450 pM, about 15 pM to about 400 pM, about 15 pM to about 350 pM, about 15 pM to about 300 pM, about 15 pM to about 250 pM, about 15 pM to about 200 pM, about 15 pM to about 150 pM, about 15 pM to about 100 pM, about 15 pM to about 90 pM, about 15 pM to about 80 pM, about 15 pM to about 70 pM, about 15 pM to about 60 pM, about 15 pM to about 50 pM, about 15 pM to about 40 pM, about 15 pM to about 30 pM, about 15 pM to about 20 pM, about 20 pM to about 30 nM, about 20 pM to about 25 nM, about 20 pM to about 20 nM, about 20 pM to about 15 nM, about 20 pM to about 10 nM, about 20 pM to about 5 nM, about 20 pM to about 2 nM, about 20 pM to about 1 nM, about 20 pM to about 950 pM, about 20 pM to about 900 pM, about 20 pM to about 850 pM, about 20 pM to about 800 pM, about 20 pM to about 750 pM, about 20 pM to about 700 pM, about 20 pM to about 650 pM, about 20 pM to about 600 pM, about 20 pM to about 550 pM, about 20 pM to about 500 pM, about 20 pM to about 450 pM, about 20 pM to about 400 pM, about 20 pM to about 350 pM, about 20 pM to about 300 pM, about 20 pM to about 250 pM, about 20 pM to about 20 pM, about 200 pM to about 150 pM, about 20 pM to about 100 pM, about 20 pM to about 90 pM, about 20 pM to about 80 pM, about 20 pM to about 70 pM, about 20 pM to about 60 pM, about 20 pM to about 50 pM, about 20 pM to about 40 pM, about 20 pM to about 30 pM, about 30 pM to about 30 nM, about 30 pM to about 25 nM, about 30 pM to about 30 nM, about 30 pM to about 15 nM, about 30 pM to about 10 nM, about 30 pM to about 5 nM, about 30 pM to about 2 nM, about 30 pM to about 1 nM, about 30 pM to about 950 pM, about 30 pM to about 900 pM, about 30 pM to about 850 pM, about 30 pM to about 800 pM, about 30 pM to about 750 pM, about 30 pM to about 700 pM, about 30 pM to about 650 pM, about 30 pM to about 600 pM, about 30 pM to about 550 pM, about 30 pM to about 500 pM, about 30 pM to about 450 pM, about 30 pM to about 400 pM, about 30 PM to about 350 pM, about 30 pM to about 300 pM, about 30 pM to about 250 pM, about 30 pM to about 200 pM, about 30 pM to about 150 pM, about 30 pM to about 100 pM, about 30 pM to about 90 pM, about 30 pM to about 80 pM, about 30 pM to about 70 pM, about 30 pM to about 60 pM, about 30 pM to about 50 pM, about 30 pM to about 40 pM, about 40 pM to about 30 nM, about 40 pM to about 25 nM, about 40 pM to about 30 nM, about 40 pM to about 15 nM, about 40 pM to about 10 nM, about 40 pM to about 5 nM, about 40 pM to about 2 nM, about 40 pM to about 1 nM, about 40 pM to about 950 pM, about 40 pM to about 900 pM, about 40 pM to about 850 pM, about 40 pM to about 800 pM, about 40 pM to about 750 pM, about 40 pM to about 700 pM, about 40 pM to about 650 pM, about 40 pM to about 600 pM, about 40 pM to about 550 pM, about 40 pM to about 500 pM, about 40 pM to about 450 pM, about 40 pM to about 400 pM, about 40 pM to about 350 pM, about 40 pM to about 300 pM, about 40 pM to about 250 pM, about 40 pM to about 200 pM, about 40 pM to about 150 pM, about 40 pM to about 100 pM, about 40 pM to about 90 pM, about 40 pM to about 80 pM, about 40 pM to about 70 pM, about 40 pM to about 60 pM, about 40 pM to about 50 pM, about 50 pM to about 30 nM, about 50 pM to about 25 nM, about 50 pM to about 30 nM, about 50 pM to about 15 nM, about 50 pM to about 10 nM, about 50 pM to about 5 nM, about 50 pM to about 2 nM, about 50 pM to about 1 nM, about 50 pM to about 950 pM, about 50 pM to about 900 pM, about 50 pM to about 850 pM, about 50 pM to about 800 pM, about 50 pM to about 750 pM, about 50 pM to about 700 pM, about 50 pM to about 650 pM, about 50 pM to about 600 pM, about 50 pM to about 550 pM, about 50 pM to about 500 pM, about 50 pM to about 450 pM, about 50 pM to about 400 pM, about 50 pM to about 350 pM, about 50 pM to about 300 PM, about 50 pM to about 250 pM, about 50 pM to about 200 pM, about 50 pM to about 150 pM, about 50 pM to about 100 pM, about 50 pM to about 90 pM, about 50 pM to about 80 pM, about 50 pM to about 70 pM, about 50 pM to about 60 pM, about 60 pM to about 30 nM, about 60 pM to about 25 nM, about 60 pM to about 30 nM, about 60 pM to about 15 nM, about 60 pM to about 10 nM, about 60 pM to about 5 nM, about 60 pM to about 2 nM, about 60 pM to about 1 nM, about 60 pM to about 950 pM, about 60 pM to about 900 pM, about 60 pM to about 850 pM, about 60 pM to about 800 pM, about 60 pM to about 750 pM, about 60 pM to about 700 pM, about 60 pM to about 650 pM, about 60 pM to about 600 pM, about 60 pM to about 550 pM, about 60 pM to about 500 pM, about 60 pM to about 450 pM, about 60 pM to about 400 pM, about 60 pM to about 350 pM, about 60 pM to about 300 pM, about 60 pM to about 250 pM, about 60 pM to about 200 pM, about 60 pM to about 150 pM, about 60 pM to about 100 pM, about 60 pM to about 90 pM, about 60 pM to about 80 pM, about 60 pM to about 70 pM, about 70 pM to about 30 nM, about 70 pM to about 25 nM, about 70 pM to about 30 nM, about 70 pM to about 15 nM, about 70 pM to about 10 nM, about 70 pM to about 5 nM, about 70 pM to about 2 nM, about 70 pM to about 1 nM, about 70 pM to about 950 pM, about 70 pM to about 900 pM, about 70 pM to about 850 pM, about 70 pM to about 800 pM, about 70 pM to about 750 pM, about 70 pM to about 700 pM, about 70 pM to about 650 pM, about 70 pM to about 600 pM, about 70 pM to about 550 pM, about 70 pM to about 500 pM, about 70 pM to about 450 pM, about 70 pM to about 400 pM, about 70 pM to about 350 pM, about 70 pM to about 300 pM, about 70 pM to about 250 pM, about 70 pM to about 200 pM, about 70 pM to about 150 pM, about 70 pM to about 100 pM, about 70 pM to about 90 pM, about 70 pM to about 80 pM, about 80 pM to about 30 nM, about 80 pM to about 25 nM, about 80 pM to about 30 nM, about 80 pM to about 15 nM, about 80 pM to about 10 nM, about 80 pM to about 5 nM, about 80 pM to about 2 nM, about 80 pM to about 1 nM, about 80 pM to about 950 pM, about 80 pM to about 900 pM, about 80 pM to about 850 pM, about 80 pM to about 800 pM, about 80 pM to about 750 pM, about 80 pM to about 700 pM, about 80 pM to about 650 pM, about 80 pM to about 600 pM, about 80 pM to about 550 pM, about 80 pM to about 500 pM, about 80 pM to about 450 pM, about 80 pM to about 400 pM, about 80 pM to about 350 pM, about 80 pM to about 300 pM, about 80 pM to about 250 pM, about 80 pM to about 200 pM, about 80 pM to about 150 pM, about 80 pM to about 100 pM, about 80 pM to about 90 pM, about 90 pM to about 30 nM, about 90 pM to about 25 nM, about 90 pM to about 30 nM, about 90 pM to about 15 nM, about 90 pM to about 10 nM, about 90 pM to about 5 nM, about 90 pM to about 2 nM, about 90 pM to about 1 nM, about 90 pM to about 950 pM, about 90 pM to about 900 pM, about 90 pM to about 850 pM, about 90 pM to about 800 pM, about 90 pM to about 750 pM, about 90 pM to about 700 pM, about 90 pM to about 650 pM, about 90 pM to about 600 pM, about 90 pM to about 550 pM, about 90 pM to about 500 pM, about 90 pM to about 450 pM, about 90 pM to about 400 pM, about 90 pM to about 350 pM, about 90 pM to about 300 pM, about 90 pM to about 250 pM, about 90 pM to about 200 pM, about 90 pM to about 150 pM, about 90 pM to about 100 pM, about 100 pM to about 30 nM, about 100 pM to about 25 nM, about 100 pM to about 30 nM, about 100 pM to about 15 nM, about 100 pM to about 10 nM, about 100 pM to about 5 nM, about 100 pM to about 2 nM, about 100 pM to about 1 nM, about 100 pM to about 950 pM, about 100 pM to about 900 pM, about 100 pM to about 850 pM, about 100 pM to about 800 pM, about 100 pM to about 750 pM, about 100 pM to about 700 pM, about 100 pM to about 650 pM, about 100 pM to about 600 pM, about 100 pM to about 550 pM, about 100 pM to about 500 pM, about 100 pM to about 450 pM, about 100 pM to about 400 pM, about 100 pM to about 350 pM, about 100 pM to about 300 pM, about 100 pM to about 250 pM, about 100 pM to about 200 pM, about 100 pM to about 150 pM, about 150 pM to about 30 nM, about 150 pM to about 25 nM, about 150 pM to about 30 nM, about 150 pM to about 15 nM, about 150 pM to about 10 nM, about 150 pM to about 5 nM, about 150 pM to about 2 nM, about 150 pM to about 1 nM, about 150 pM to about 950 pM, about 150 pM to about 900 pM, about 150 pM to about 850 pM, about 150 pM to about 800 pM, about 150 pM to about 750 pM, about 150 pM to about 700 pM, about 150 pM to about 650 pM, about 150 pM to about 600 pM, about 150 pM to about 550 pM, about 150 pM to about 500 pM, about 150 pM to about 450 pM, about 150 pM to about 400 pM, about 150 pM to about 350 pM, about 150 pM to about 300 pM, about 150 pM to about 250 pM, about 150 pM to about 200 pM, about 200 pM to about 30 nM, about 200 pM to about 25 nM, about 200 pM to about 30 nM, about 200 pM to about 15 nM, about 200 pM to about 10 nM, about 200 pM to about 5 nM, about 200 pM to about 2 nM, about 200 pM to about 1 nM, about 200 pM to about 950 pM, about 200 pM to about 900 pM, about 200 pM to about 850 pM, about 200 pM to about 800 pM, about 200 pM to about 750 pM, about 200 pM to about 700 pM, about 200 pM to about 650 pM, about 200 pM to about 600 pM, about 200 pM to about 550 pM, about 200 pM to about 500 pM, about 200 pM to about 450 pM, about 200 pM to about 400 pM, about 200 pM to about 350 pM, about 200 pM to about 300 pM, about 200 pM to about 250 pM, about 300 pM to about 30 nM, about 300 pM to about 25 nM, about 300 pM to about 30 nM, about 300 pM to about 15 nM, about 300 pM to about 10 nM, about 300 pM to about 5 nM, about 300 pM to about 2 nM, about 300 pM to about 1 nM, about 300 pM to about 950 pM, about 300 pM to about 900 pM, about 300 pM to about 850 pM, about 300 pM to about 800 pM, about 300 pM to about 750 pM, about 300 pM to about 700 pM, about 300 pM to about 650 pM, about 300 pM to about 600 pM, about 300 pM to about 550 pM, about 300 pM to about 500 pM, about 300 pM to about 450 pM, about 300 pM to about 400 pM, about 300 pM to about 350 pM, about 400 pM to about 30 nM, about 400 pM to about 25 nM, about 400 pM to about 30 nM, about 400 pM to about 15 nM, about 400 pM to about 10 nM, about 400 pM to about 5 nM, about 400 pM to about 2 nM, about 400 pM to about 1 nM, about 400 pM to about 950 pM, about 400 pM to about 900 pM, about 400 pM to about 850 pM, about 400 pM to about 800 pM, about 400 pM to about 750 pM, about 400 pM to about 700 pM, about 400 pM to about 650 pM, about 400 pM to about 600 pM, about 400 pM to about 550 pM, about 400 pM to about 500 pM, about 500 pM to about 30 nM, about 500 pM to about 25 nM, about 500 pM to about 30 nM, about 500 pM to about 15 nM, about 500 pM to about 10 nM, about 500 pM to about 5 nM, about 500 pM to about 2 nM, about 500 pM to about 1 nM, about 500 pM to about 950 pM, about 500 pM to about 900 pM, about 500 pM to about 850 pM, about 500 pM to about 800 pM, about 500 pM to about 750 pM, about 500 pM to about 700 pM, about 500 pM to about 650 pM, about 500 pM to about 600 pM, about 500 pM to about 550 pM, about 600 pM to about 30 nM, about 600 pM to about 25 nM, about 600 pM to about 30 nM, about 600 pM to about 15 nM, about 600 pM to about 10 nM, about 600 pM to about 5 nM, about 600 pM to about 2 nM, about 600 pM to about 1 nM, about 600 pM to about 950 pM, about 600 pM to about 900 pM, about 600 pM to about 850 pM, about 600 pM to about 800 pM, about 600 pM to about 750 pM, about 600 pM to about 700 pM, about 600 pM to about 650 pM, about 700 pM to about 30 nM, about 700 pM to about 25 nM, about 700 pM to about 30 nM, about 700 pM to about 15 nM, about 700 pM to about 10 nM, about 700 pM to about 5 nM, about 700 pM to about 2 nM, about 700 pM to about 1 nM, about 700 pM to about 950 pM, about 700 pM to about 900 pM, about 700 pM to about 850 pM, about 700 pM to about 800 pM, about 700 pM to about 750 pM, about 800 pM to about 30 nM, about 800 pM to about 25 nM, about 800 pM to about 30 nM, about 800 pM to about 15 nM, about 800 pM to about 10 nM, about 800 pM to about 5 nM, about 800 pM to about 2 nM, about 800 pM to about 1 nM, about 800 pM to about 950 pM, about 800 pM to about 900 pM, about 800 pM to about 850 pM, about 900 pM to about 30 nM, about 900 pM to about 25 nM, about 900 pM to about 30 nM, about 900 pM to about 15 nM, about 900 pM to about 10 nM, about 900 pM to about 5 nM, about 900 pM to about 2 nM, about 900 pM to about 1 nM, about 900 pM to about 950 pM, about 1 nM to about 30 nM, about 1 nM to about 25 nM, about 1 nM to about 20 nM, about 1 nM to about 15 nM, about 1 nM to about 10 nM, about 1 nM to about 5 nM, about 2 nM to about 30 nM, about 2 nM to about 25 nM, about 2 nM to about 20 nM, about 2 nM to about 15 nM, about 2 nM to about 10 nM, about 2 nM to about 5 nM, about 4 nM to about 30 nM, about 4 nM to about 25 nM, about 4 nM to about 20 nM, about 4 nM to about 15 nM, about 4 nM to about 10 nM, about 4 nM to about 5 nM, about 5 nM to about 30 nM, about 5 nM to about 25 nM, about 5 nM to about 20 nM, about 5 nM to about 15 nM, about 5 nM to about 10 nM, about 10 nM to about 30 nM, about 10 nM to about 25 nM, about 10 nM to about 20 nM, about 10 nM to about 15 nM, about 15 nM to about 30 nM, about 15 nM to about 25 nM, about 15 nM to about 20 nM, about 20 nM to about 30 nM, and about 20 nM to about 25 nM).

Any of the target-binding domains described herein can bind to its target with a K_{D} of between about 1 nM to about 10 nM (e.g., about 1 nM to about 9 nM, about 1 nM to about 8 nM, about 1 nM to about 7 nM, about 1 nM to about 6 nM, about 1 nM to about 5 nM, about 1 nM to about 4 nM, about 1 nM to about 3 nM, about 1 nM to about 2 nM, about 2 nM to about 10 nM, about 2 nM to about 9 nM, about 2 nM to about 8 nM, about 2 nM to about 7 nM, about 2 nM to about 6 nM, about 2 nM to about 5 nM, about 2 nM to about 4 nM, about 2 nM to about 3 nM, about 3 nM to about 10 nM, about 3 nM to about 9 nM, about 3 nM to about 8 nM, about 3 nM to about 7 nM, about 3 nM to about 6 nM, about 3 nM to about 5 nM, about 3 nM to about 4 nM, about 4 nM to about 10 nM, about 4 nM to about 9 nM, about 4 nM to about 8 nM, about 4 nM to about 7 nM, about 4 nM to about 6 nM, about 4 nM to about 5 nM, about 5 nM to about 10 nM, about 5 nM to about 9 nM, about 5 nM to about 8 nM, about 5 nM to about 7 nM, about 5 nM to about 6 nM, about 6 nM to about 10 nM, about 6 nM to about 9 nM, about 6 nM to about 8 nM, about 6 nM to about 7 nM, about 7 nM to about 10 nM, about 7 nM to about 9 nM, about 7 nM to about 8 nM, about 8 nM to about 10 nM, about 8 nM to about 9 nM, and about 9 nM to about 10 nM).

A variety of different methods known in the art can be used to determine the K_{D} values of any of the antigen-binding protein constructs described herein (e.g., an electrophoretic mobility shift assay, a filter binding assay, surface plasmon resonance, and a biomolecular binding kinetics assay, etc.).

### Antigen-Binding Domains

In some embodiments of any of the multi-chain chimeric polypeptides described herein, the first target-binding domain and the second target-binding domain bind specifically to the same antigen. In some embodiments of these multi-chain chimeric polypeptides, the first target-binding domain and the second target-binding domain bind specifically to the same epitope. In some embodiments of these multi-chain chimeric polypeptides, the first target-binding domain and the second target-binding domain include the same amino acid sequence.

In some embodiments of any of the multi-chain chimeric polypeptides described herein, the first target-binding domain and the second target-binding domain bind specifically to different antigens.

In some embodiments of any of the multi-chain chimeric polypeptides described herein, one or both of the first target-binding domain and the second target-binding domain is an antigen-binding domain. In some embodiments of any of the multi-chain chimeric polypeptides described herein, the first target-binding domain and the second target-binding domain are each antigen-binding domains.

In some embodiments of any of the multi-chain chimeric polypeptides described herein, the antigen-binding domain includes or is a scFv or a single domain antibody (e.g., a VHH or a VNAR domain).

In some examples, the first and/or second target-binding domain is an antigen-binding domain. Non-limiting examples of antigen-binding domains that can bind specifically to a ligand of a ligand of TGF-βRII include the antigen-binding domains of SAR-439459, NIS793, SRK-181, and GC1008 (fresolimumab).

In some examples, the one or more additional target-binding domains can be an antigen-binding domain (e.g., any of the antigen-binding domains described herein) that binds specifically to any one of CD16a (see, e.g., those described in U.S. Pat. No. 9,035,026), CD28 (see, e.g., those described in U.S. Pat. No. 7,723,482), CD3 (see, e.g., those described in U.S. Pat. No. 9,226,962), CD33 (see, e.g., those described in U.S. Pat. No. 8,759,494), CD20 (see, e.g., those described in WO 2014/026054), CD19 (see, e.g., those described in U.S. Pat. No. 9,701,758), CD22 (see, e.g., those described in WO 2003/104425), CD123 (see, e.g., those described in WO 2014/130635), IL-1R (see, e.g., those described in U.S. Pat. No. 8,741,604), IL-1 (see, e.g., those described in WO 2014/095808), VEGF (see, e.g., those described in U.S. Pat. No. 9,090,684), IL-6R (see, e.g., those described in U.S. Pat. No. 7,482,436), IL-4 (see, e.g., those described in U.S. Patent Application Publication No. 2012/0171197), IL-10 (see, e.g., those described in U.S. Patent Application Publication No. 2016/0340413), PDL-1 (see, e.g., those described in Drees et al., Protein Express. Purif. 94:60-66, 2014), TIGIT (see, e.g., those described in U.S. Patent Application Publication No. 2017/0198042), PD-1 (see, e.g., those described in U.S. Pat. No. 7,488,802), TIM3 (see, e.g., those described in U.S. Pat. No. 8,552,156), CTLA4 (see, e.g., those described in WO 2012/120125), MICA (see, e.g., those described in WO 2016/154585), MICB (see, e.g., those described in U.S. Pat. No. 8,753,640), IL-6 (see, e.g., those described in Gejima et al., Human Antibodies 11 (4): 121-129, 2002), IL-8 (see, e.g., those described in U.S. Pat. No. 6,117,980), TNFα (see, e.g., those described in Geng et al., Immunol. Res. 62 (3): 377-385, 2015), CD26a (see, e.g., those described in WO 2017/189526), CD36 (see, e.g., those described in U.S. Patent Application Publication No. 2015/0259429), ULBP2 (see, e.g., those described in U.S. Pat. No. 9,273,136), CD30 (see, e.g., those described in Homach et al., Scand. J. Immunol. 48 (5): 497-501, 1998), CD200 (see, e.g., those described in U.S. Pat. No. 9,085,623), IGF-1R (see, e.g., those described in U.S. Patent Application Publication No. 2017/0051063), MUC4AC (see, e.g., those described in WO 2012/170470), MUC5AC (see, e.g., those described in U.S. Pat. No. 9,238,084), Trop-2 (see, e.g., those described in WO 2013/068946), CMET (see, e.g., those described in Edwardraja et al., Biotechnol. Bioeng. 106 (3): 367-375, 2010), EGFR (see, e.g., those described in Akbari et al., Protein Expr. Purif. 127:8-15, 2016), HER1 (see, e.g., those described in U.S. Patent Application Publication No. 2013/0274446), HER2 (see, e.g., those described in Cao et al., Biotechnol. Lett. 37 (7): 1347-1354, 2015), HER3 (see, e.g., those described in U.S. Pat. No. 9,505,843), PSMA (see, e.g., those described in Parker et al., Protein Expr. Purif. 89 (2): 136-145, 2013), CEA (see, e.g., those described in WO 1995/015341), B7H3 (see, e.g., those described in U.S. Pat. No. 9,371,395), EPCAM (see, e.g., those described in WO 2014/159531), BCMA (see, e.g., those described in Smith et al., Mol. Ther. 26 (6): 1447-1456, 2018), P-cadherin (see, e.g., those described in U.S. Pat. No. 7,452,537), CEACAM5 (see, e.g., those described in U.S. Pat. No. 9,617,345), a UL16-binding protein (see, e.g., those described in WO 2017/083612), HLA-DR (see, e.g., Pistillo et al., Exp. Clin. Immunogenet. 14 (2): 123-130, 1997), DLL4 (see, e.g., those described in WO 2014/007513), TYRO3 (see, e.g., those described in WO 2016/166348), AXL (see, e.g., those described in WO 2012/175692), MER (see, e.g., those described in WO 2016/106221), CD122 (see, e.g., those described in U.S. Patent Application Publication No. 2016/0367664), CD155 (see, e.g., those described in WO 2017/149538), or PDGF-DD (see, e.g., those described in U.S. Pat. No. 9,441,034).

The antigen-binding domains present in any of the multi-chain chimeric polypeptides described herein are each independently selected from the group consisting of: a VHH domain, a VNAR domain, and a scFv. In some embodiments, any of the antigen-binding domains described herein is a BiTe, a (scFv)₂, a nanobody, a nanobody-HSA, a DART, a TandAb, a scDiabody, a scDiabody-CH3, scFv-CH-CL-scFv, a HSAbody, scDiabody-HAS, or a tandem-scFv. Additional examples of antigen-binding domains that can be used in any of the multi-chain chimeric polypeptide are known in the art.

A VHH domain is a single monomeric variable antibody domain that can be found in camelids. A VNAR domain is a single monomeric variable antibody domain that can be found in cartilaginous fish. Non-limiting aspects of VHH domains and VNAR domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr. Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments, each of the antigen-binding domains in the multi-chain chimeric polypeptides described herein are both VHH domains, or at least one antigen-binding domain is a VHH domain. In some embodiments, each of the antigen-binding domains in the multi-chain chimeric polypeptides described herein are both VNAR domains, or at least one antigen-binding domain is a VNAR domain. In some embodiments, each of the antigen-binding domains in the multi-chain chimeric polypeptides described herein are both scFv domains, or at least one antigen-binding domain is a scFv domain.

In some embodiments, two or more of polypeptides present in the multi-chain chimeric polypeptide can assemble (e.g., non-covalently assemble) to form any of the antigen-binding domains described herein, e.g., an antigen-binding fragment of an antibody (e.g., any of the antigen-binding fragments of an antibody described herein), a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a kλ-body, an orthogonal Fab, a DVD-IgG, a IgG (H)-scFv, a scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv₂, a scFv-KIH, a Fab-scFv-Fc, a tetravalent HCAb, a scDiabody-Fc, a Diabody-Fc, a tandem scFv-Fc, an Intrabody, a dock and lock, a lmmTAC, an IgG-IgG conjugate, a Cov-X-Body, and a scFv₁-PEG-scFv₂. See, e.g., Spiess et al., Mol. Immunol. 67:95-106, 2015, incorporated in its entirety herewith, for a description of these elements. Non-limiting examples of an antigen-binding fragment of an antibody include an Fv fragment, a Fab fragment, a F(ab′)₂ fragment, and a Fab′ fragment. Additional examples of an antigen-binding fragment of an antibody is an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

An “Fv” fragment includes a non-covalently-linked dimer of one heavy chain variable domain and one light chain variable domain.

A “Fab” fragment includes the constant domain of the light chain and the first constant domain (CH1) of the heavy chain, in addition to the heavy and light chain variable domains of the Fv fragment.

A “F(ab′)₂” fragment includes two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

DARTs are described in, e.g., Garber, Nature Reviews Drug Discovery 13:799-801, 2014.

In some embodiments of any of the antigen-binding domains described herein can bind to an antigen selected from the group consisting of: a protein, a carbohydrate, a lipid, and a combination thereof.

Additional examples and aspects of antigen-binding domains are known in the art.

### Soluble Interleukin or Cytokine Protein

In some embodiments of any of the multi-chain chimeric polypeptides described herein, one or more additional target-binding domains can be a soluble interleukin protein or soluble cytokine protein. In some embodiments, the soluble interleukin or soluble cytokine protein is selected from the group of: IL-2, IL-3, IL-7, IL-8, IL-10, IL-12, IL-15, IL-17, IL-18, IL-21, PDGF-DD, SCF, and FLT3L. Non-limiting examples of soluble IL-2, IL-3, IL-7, IL-8, IL-10, IL-15, IL-17, IL-18, IL-21, PDGF-DD, SCF, and FLT3L are provided below.

Human Soluble IL-2
(SEQ ID NO: 10)
aptssstkkt qlqlehllld lqmilnginn yknpkltrml tfkfympkka
telkhlqcle eelkpleevl nlaqsknfhl rprdlisnin vivlelkgse
ttfmceyade tativeflnr witfcqsiis tlt
Human Soluble IL-3
(SEQ ID NO: 11)
apmtqttplkt swvncsnmid eiithlkqpp lplldfnnln gedqdilmen
nlrrpnleaf nravkslgna saiesilknl lpclplataa ptrhpihikd
gdwnefrrkl tfylktlena qaqqttlsla if
Human Soluble IL-7
(SEQ ID NO: 12)
dcdiegkdgkqyesv lmvsidqlld smkeigsncl nnefnffkrh icdankegmf
lfraarklrq flkmnstgdf dlhllkvseg ttillnctgq vkgrkpaalg
eaqptkslee nkslkeqkkl ndlcflkrll qeiktcwnki lmgtkeh
Human Soluble IL-8
(SEQ ID NO: 13)
egavlprsak elrcqcikty skpfhpkfik elrviesgph canteiivkl
sdgrelcldp kenwvqrvve kflkraens
Human Soluble IL-10
(SEQ ID NO: 14)
spgqgtqsensc thfpgnlpnm lrdlrdafsr vktffqmkdq ldnlllkesl
ledfkgylgc qalsemiqfy leevmpqaen qdpdikahvn slgenlktlr
lrlrrchrfl pcenkskave qvknafnklq ekgiykamse fdifinyiea
ymtmkirn
Human Soluble IL-15
(SEQ ID NO: 15)
Nwvnvisdlkki edliqsmhid atlytesdvh psckvtamkc fllelqvisl
esgdasihdt venliilann slssngnvte sgckeceele eknikeflqs
fvhivqmfin ts
Human Soluble IL-17
(SEQ ID NO: 16)
gitiprn pgcpnsedkn fprtvmvnln ihnrntntnp krssdyynrs
tspwnlhrne dperypsviw eakcrhlgci nadgnvdyhm nsvpiqqeil
vlrrepphcp nsfrlekilv svgctcvtpi vhhva
Human Soluble IL-18
(SEQ ID NO: 17)
yfgklesklsvirn lndqvlfidq gnrplfedmt dsdcrdnapr tifiismykd
sqprgmavti svkcekistl scenkiisfk emnppdnikd tksdiiffqr
svpghdnkmq fesssyegyf lacekerdlf klilkkedel gdrsimftvq ned
Human Soluble PDGF-DD
(SEQ ID NO: 18)
rdtsatpqsasi kalrnanlrr desnhltdly rrdetiqvkg ngyvqsprfp
nsyprnlllt wrlhsqentr iqlvfdnqfg leeaendicr ydfvevedis
etstiirgrw cghkevppri ksrtnqikit fksddyfvak pgfkiyysll
edfqpaaase tnwesvtssi sgvsynspsv tdptliadal dkkiaefdtv
edllkyfnpe swqedlenmy ldtpryrgrs yhdrkskvdl drlnddakry
sctprnysvn ireelklanv vffprcllvq rcggncgcgt vnwrsctcns
gktvkkyhev lqfepghikr rgraktmalv diqldhherc dcicssrppr
Human Soluble SCF
(SEQ ID NO: 19)
egicrnrvtnnvkdv tklvanlpkd ymitlkyvpg mdvlpshcwi semvvqlsds
ltdlldkfsn iseglsnysi idklvnivdd lvecvkenss kdlkksfksp
eprlftpeef frifnrsida fkdfvvaset sdcvvsstls pekdsrvsvt
kpfmlppvaa sslrndssss nrkaknppgd sslhwaamal palfsliigf
afgalywkkr qpsltraven iqineednei smlqekeref qev
Human Soluble FLT3L
(SEQ ID NO: 20)
tqdcsfqhspissd favkirelsd yllqdypvtv asnlqdeelc gglwrlvlaq
rwmerlktva gskmqgller vnteihfvtk cafqpppscl rfvqtnisrl
lqetseqlva lkpwitrqnf srclelqcqp dsstlpppws prpleatapt
apqpplllll llpvglllla aawclhwqrt rrrtprpgeq vppvpspqdl
llveh

Non-limiting examples of soluble MICA, MICB, ULBP1, ULBP2, ULBP3, ULBP4, ULBP5, and ULBP6 are provided below.

Human Soluble MICA
(SEQ ID NO: 21)
ephslry nltvlswdgs vqsgfltevh ldgqpflrcd rqkcrakpqg
qwaedvlgnk twdretrdlt gngkdlrmtl ahikdqkegl hslqeirvce
ihednstrss qhfyydgelf lsqnletkew tmpqssraqt lamnvrnflk
edamktkthy hamhadclqe lrrylksgvv lrrtvppmvn vtrseasegn
itvtcrasgf ypwnitlswr qdgvslshdt qqwgdvlpdg ngtyqtwvat
ricqgeeqrf tcymehsgnh sthpvpsgkv lvlqshwqtf hvsavaaaai
fviiifyvrc ckkktsaaeg pelvslqvld qhpvgtsdhr datqlgfqpl
msdlgstgst ega
Human Soluble MICB
(SEQ ID NO: 22)
aephslry nlmvlsqdes vqsgflaegh ldgqpflryd rqkrrakpqg
qwaedvlgak twdtetedlt engqdlrrtl thikdqkggl hslqeirvce
ihedsstrgs rhfyydgelf lsqnletes tvpqssraqt lamnvtnfwk
edamktkthy ramqadclqk lqrylksgva irrtvppmvn vtcsevsegn
itvtcrassf yprnitltwr qdgvslshnt qqwgdvlpdg ngtyqtwvat
rirqgeeqrf tcymehsgnh gthpvpsgkv lvlqsqrtdf pyvsaampcf
viiiilcvpc ckkktsaaeg pelvslqvld qhpvgtgdhr
daaqlgfqpl msatgstgst ega
Human Soluble ULBP1
(SEQ ID NO: 23)
wvdthclcydfiit pksrpepqwc evqglvderp flhydcvnhk akafaslgkk
vnvtktweeq tetlrdvvdf 1kgqlldiqv enlipieplt lqarmscehe
ahghgrgswq flingqkfll fdsnnrkwta lhpgakkmte kweknrdvtm
ffqkislgdc kmwleeflmy weqmldptkp pslapg
Human Soluble ULBP2
(SEQ ID NO: 24)
gradphslcyditvi pkfrpgprwc avqgqvdekt flhydcgnkt vtpvsplgkk
lnvttawkaq npvlrevvdi lteqlrdiql enytpkeplt lqarmsceqk
aeghssgswq fsfdgqifll fdsekrmwtt vhpgarkmke kwendkvvam
sfhyfsmgdc igwledflmg mdstlepsag aplams
Human Soluble ULBP3
(SEQ ID NO: 25)
dahslwynfti ihlprhgqqw cevqsqvdqk nflsydcgsd kvlsmghlee
qlyatdawgk qlemlrevgq rlrleladte ledftpsgpl tlqvrmscec
eadgyirgsw qfsfdgrkfl lfdsnnrkwt vvhagarrmk ekwekdsglt
Human Soluble ULBP5
(SEQ ID NO: 27)
gladp hslcyditvi pkfrpgprwc avqgqvdekt flhydcgskt
vtpvsplgkk lnvttawkaq npvlrevvdi lteqlldiql enyipkeplt
lqarmsceqk aeghgsgswq lsfdgqifll fdsenrmwtt vhpgarkmke
kwendkdmtm sfhyismgdc tgwledflmg mdstlepsag apptmssg
Human Soluble ULBP6
(SEQ ID NO: 28)
rrddp hslcyditvi pkfrpgprwc avqgqvdekt flhydcgnkt
vtpvsplgkk lnvtmawkaq npvlrevvdi lteqlldiql enytpkeplt
lqarmsceqk aeghssgswq fsidgqtfll fdsekrmwtt vhpgarkmke
kwendkdvam sfhyismgdc igwledflmg mdstlepsag aplamssg
tffkmvsmrd ckswirdflm hrkkrlepta pptmapg
Human Soluble ULBP4
(SEQ ID NO: 26
hslcfnftik slsrpgqpwc eaqvflnknl flqynsdnnm vkplgllgkk
vyatstwgel tqtlgevgrd lrmllcdikp qiktsdpstl qvemfcqrea
erctgaswqf atngeksllf damnmtwtvi nheaskiket wkkdrgleky
frklskgdcd hwlreflghw eampeptvsp vnasdihwss sslpdrwiil
gafillvlmg ivlicvwwqn gewqaglwpl rts

Additional examples of soluble interleukin proteins and soluble cytokine proteins are known in the art.

### Soluble Receptor

In some embodiments of any of the multi-chain chimeric polypeptides described herein, one or both of the first target-binding domain and the second target-binding domain is a soluble interleukin receptor or a soluble cytokine receptor or a ligand receptor. In some embodiments, the first and/or second target-binding domains can be a soluble TGF-β receptor II (TGF-β RII) (see, e.g., those described in Yung et al., Am. J. Resp. Crit. Care Med. 194 (9): 1140-1151, 2016).

In some embodiments, the first target-binding domain includes a soluble TGF-β receptor (e.g., a soluble TGFRβRII (e.g., a soluble human TGFRβRII)). In some embodiments, the second target-binding domain includes a soluble TGF-β receptor (e.g., a soluble TGFRβRII (e.g., a soluble human TGFRβRII)). In some embodiments, the soluble human TGFRβRII includes a first sequence of soluble human TGFRβRII and a second sequence of soluble human TGFRβRII. In some embodiments of these multi-chain chimeric polypeptides, the soluble human TGFRβRII includes a linker disposed between the first sequence of soluble human TGFRβRII and the second sequence of soluble human TGFRβRII. In some examples of these multi-chain chimeric polypeptides, the linker includes the sequence GGGGSGGGGSGGGGS (SEQ ID NO: 7).

In some embodiments, the first sequence of soluble human TGFRβRII comprises a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 66)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPD.

In some embodiments, the second sequence of soluble human TGFRβRII comprises a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 66)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPD.

In some embodiments, the first sequence of soluble human TGFRβRII is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 67)
ATTCCTCCCCACGTGCAGAAGAGCGTGAATAATGACATGATCGTGACCG
ATAACAATGGCGCCGTGAAATTTCCCCAGCTGTGCAAATTCTGCGATGT
GAGGTTTTCCACCTGCGACAACCAGAAGTCCTGTATGAGCAACTGCACA
ATCACCTCCATCTGTGAGAAGCCTCAGGAGGTGTGCGTGGCTGTCTGGC
GGAAGAATGACGAGAATATCACCCTGGAAACCGTCTGCCACGATCCCAA
GCTGCCCTACCACGATTTCATCCTGGAAGACGCCGCCAGCCCTAAGTGC
ATCATGAAAGAGAAAAAGAAGCCTGGCGAGACCTTTTTCATGTGCTCCT
GCAGCAGCGACGAATGCAACGACAATATCATCTTTAGCGAGGAATACAA
TACCAGCAACCCCGAC.

In some embodiments, the second sequence of soluble human TGFRβRII is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 67)
ATTCCTCCCCACGTGCAGAAGAGCGTGAATAATGACATGATCGTGACCG
ATAACAATGGCGCCGTGAAATTTCCCCAGCTGTGCAAATTCTGCGATGT
GAGGTTTTCCACCTGCGACAACCAGAAGTCCTGTATGAGCAACTGCACA
ATCACCTCCATCTGTGAGAAGCCTCAGGAGGTGTGCGTGGCTGTCTGGC
GGAAGAATGACGAGAATATCACCCTGGAAACCGTCTGCCACGATCCCAA
GCTGCCCTACCACGATTTCATCCTGGAAGACGCCGCCAGCCCTAAGTGC
ATCATGAAAGAGAAAAAGAAGCCTGGCGAGACCTTTTTCATGTGCTCCT
GCAGCAGCGACGAATGCAACGACAATATCATCTTTAGCGAGGAATACAA
TACCAGCAACCCCGAC.

In some embodiments, the soluble human TGFRβRII is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 68)
ATCCCCCCCCATGTGCAAAAGAGCGTGAACAACGATATGATCGTGACCG
ACAACAACGGCGCCGTGAAGTTTCCCCAGCTCTGCAAGTTCTGCGATGT
CAGGTTCAGCACCTGCGATAATCAGAAGTCCTGCATGTCCAACTGCACG
ATCACCTCCATCTGCGAGAAGCCCCAAGAAGTGTGCGTGGCCGTGTGGC
GGAAAAATGACGAGAACATCACCCTGGAGACCGTGTGTCACGACCCCAA
GCTCCCTTATCACGACTTCATTCTGGAGGACGCTGCCTCCCCCAAATGC
ATCATGAAGGAGAAGAAGAAGCCCGGAGAGACCTTCTTTATGTGTTCCT
GTAGCAGCGACGAGTGTAACGACAACATCATCTTCAGCGAAGAGTACAA
CACCAGCAACCCTGATGGAGGTGGCGGATCCGGAGGTGGAGGTTCTGGT
GGAGGTGGGAGTATTCCTCCCCACGTGCAGAAGAGCGTGAATAATGACA
TGATCGTGACCGATAACAATGGCGCCGTGAAATTTCCCCAGCTGTGCAA
ATTCTGCGATGTGAGGTTTTCCACCTGCGACAACCAGAAGTCCTGTATG
AGCAACTGCACAATCACCTCCATCTGTGAGAAGCCTCAGGAGGTGTGCG
TGGCTGTCTGGCGGAAGAATGACGAGAATATCACCCTGGAAACCGTCTG
CCACGATCCCAAGCTGCCCTACCACGATTTCATCCTGGAAGACGCCGCC
AGCCCTAAGTGCATCATGAAAGAGAAAAAGAAGCCTGGCGAGACCTTTT
TCATGTGCTCCTGCAGCAGCGACGAATGCAACGACAATATCATCTTTAG
CGAGGAATACAATACCAGCAACCCCGAC.

In some embodiments, the soluble human TGFβRII includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 69)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPD.

In some embodiments, the one or more additional target-binding domains can be a soluble TGF-β receptor II (TGF-β RII) (see, e.g., those described in Yung et al., Am. J. Resp. Crit. Care Med. 194 (9): 1140-1151, 2016), a soluble TGF-βRIII (see, e.g., those described in Heng et al., Placenta 57:320, 2017), a soluble NKG2D (see, e.g., Cosman et al., Immunity 14 (2): 123-133, 2001; Costa et al., Front. Immunol., Vol. 9, Article 1150 May 29, 2018; doi: 10.3389/fimmu.2018.01150), a soluble NKD30 (see, e.g., Costa et al., Front. Immunol., Vol. 9, Article 1150 May 29, 2018; doi: 10.3389/fimmu.2018.01150), a soluble NKD44 (see, e.g., those described in Costa et al., Front. Immunol., Vol. 9, Article 1150 May 29, 2018; doi: 10.3389/fimmu.2018.01150), a soluble NKD46 (see, e.g., Mandelboim et al., Nature 409:1055-1060, 2001; Costa et al., Front. Immunol., Vol. 9, Article 1150 May 29, 2018; doi: 10.3389/fimmu.2018.01150), a soluble DNAM-1 (see, e.g., those described in Costa et al., Front. Immunol., Vol. 9, Article 1150 May 29, 2018; doi: 10.3389/fimmu.2018.01150), a scMHCI (see, e.g., those described in Washburn et al., PLOS One 6 (3): e18439, 2011), a scMHCII (see, e.g., those described in Bishwajit et al., Cellular Immunol. 170 (1): 25-33, 1996), a scTCR (see, e.g., those described in Weber et al., Nature 356 (6372): 793-796, 1992), a soluble CD155 (see, e.g., those described in Tahara-Hanaoka et al., Int. Immunol. 16 (4): 533-538, 2004), or a soluble CD28 (see, e.g., Hebbar et al., Clin. Exp. Immunol. 136:388-392, 2004).

Additional examples of soluble interleukin receptors and soluble cytokine receptors are known in the art.

### Additional Target-Binding Domains

In some embodiments of any of the multi-chain chimeric polypeptides, the first chimeric polypeptide further includes one or more (e.g., two, three, four, five, six, seven, eight, nine, or ten) additional target-binding domain(s) (e.g., any of the exemplary target-binding domains described herein or known in the art), where at least one of the one or more additional antigen-binding domain(s) is positioned between the soluble tissue factor domain (e.g., any of the exemplary soluble tissue factor domains described herein or known in the art) and the first domain of the pair of affinity domains (e.g., any of the exemplary first domains of any of the exemplary pairs of affinity domains described herein). In some embodiments, the first chimeric polypeptide can further include a linker sequence (e.g., any of the exemplary linker sequences described herein or known in the art) between the soluble tissue factor domain (e.g., any of the exemplary soluble tissue factor domains described herein) and the at least one of the one or more additional target-binding domain(s) (e.g., any of the exemplary target-binding domains described herein or known in the art), and/or a linker sequence (e.g., any of the exemplary linker sequences described herein or known in the art) between the at least one of the one or more additional target-binding domain(s) (e.g., any of the exemplary target-binding domains described herein or known in the art) and the first domain of the pair of affinity domains (e.g., any of the exemplary first domains described herein of any of the exemplary pairs of affinity domains described herein).

In some embodiments of any of the multi-chain chimeric polypeptides described herein, the first chimeric polypeptide further includes one or more (e.g., two, three, four, five, six, seven, eight, nine, or ten) additional target-binding domains at the N-terminal and/or C-terminal end of the first chimeric polypeptide. In some embodiments, at least one of the one or more additional target-binding domains (e.g., any of the exemplary target-binding domains described herein or known in the art) directly abuts the first domain of the pair of affinity domains (e.g., any of the exemplary first domains described herein of any of the exemplary pairs of affinity domains described herein) in the first chimeric polypeptide. In some embodiments, the first chimeric polypeptide further includes a linker sequence (e.g., any of the exemplary linker sequences described herein or known in the art) between the at least one of the one or more additional target-binding domains (e.g., any of the exemplary target-binding domains described herein or known in the art) and the first domain of the pair of affinity domains (e.g., any of the exemplary first domains described herein of any of the exemplary pairs of affinity domains described herein). In some embodiments, the at least one of the one or more additional target-binding domains (e.g., any of the exemplary target-binding domains described herein or known in the art) directly abuts the first target-binding domain (e.g., any of the exemplary target-binding domains described herein or known in the art) in the first chimeric polypeptide. In some embodiments, the first chimeric polypeptide further comprises a linker sequence (e.g., any of the exemplary linker sequences described herein or known in the art) between the at least one of the one or more additional target-binding domains (e.g., any of the exemplary target-binding domains described herein or known in the art) and the first target-binding domain (e.g., any of the exemplary target-binding domains described herein or known in the art).

In some embodiments of any of the multi-chain chimeric polypeptides described herein, at least one of the one or more additional target-binding domains (e.g., any of the exemplary target-binding domains described herein or known in the art) is disposed at the N- and/or C-terminus of the first chimeric polypeptide, and at least one of the one or more additional target-binding domains (e.g., any of the exemplary target-binding domains described herein or known in the art) is positioned between the soluble tissue factor domain (e.g., any of the exemplary soluble tissue factor domains described herein or known in the art) and the first domain of the pair of affinity domains (e.g., any of the exemplary first domains of any of the exemplary pairs of affinity domains described herein) in the first chimeric polypeptide. In some embodiments, the at least one additional target-binding domain (e.g., any of the exemplary target-binding domains described herein or known in the art) of the one or more additional target-binding domains disposed at the N-terminus directly abuts the first target-binding domain (e.g., any of the exemplary target-binding domains described herein or known in the art) or the first domain of the pair of affinity domains (e.g., any of the exemplary first domains described herein of any of the exemplary pairs of affinity domains described herein) in the first chimeric polypeptide. In some embodiments, the first chimeric polypeptide further comprises a linker sequence (e.g., any of the linker sequences described herein or known in the art) disposed between the at least one additional target-binding domain (e.g., any of the exemplary target-binding domains described herein or known in the art) and the first target-binding domain (e.g., any of the exemplary target-binding domains described herein or known in the art) or the first domain of the pair of affinity domains (e.g., any of the exemplary first domains described herein of any of the exemplary pairs of affinity domains described herein) in the first chimeric polypeptide. In some embodiments, the at least one additional target-binding domain (e.g., any of the exemplary target-binding domains described herein or known in the art) of the one or more additional target-binding domains disposed at the C-terminus directly abuts the first target-binding domain (e.g., any of the exemplary target-binding domains described herein or known in the art) or the first domain of the pair of affinity domains (e.g., any of the exemplary first domains of any of the exemplary pairs of affinity domains described herein) in the first chimeric polypeptide. In some embodiments, the first chimeric polypeptide further includes a linker sequence (e.g., any of the exemplary linker sequences described herein or known in the art) disposed between the at least one additional target-binding domain (e.g., any of the exemplary target-binding domains described herein or known in the art) and the first target-binding domain (e.g., any of the exemplary target-binding domains described herein or known in the art) or the first domain of the pair of affinity domains (e.g., any of the exemplary first domains described herein of any of the exemplary pairs of affinity domains described herein) in the first chimeric polypeptide. In some embodiments, the at least one of the one or more additional target-binding domains (e.g., any of the exemplary target-binding domains described herein or known in the art) positioned between the soluble tissue factor domain (e.g., any of the exemplary soluble tissue factor domains described herein) and the first domain of the pair of affinity domains (e.g., any of the first domains described herein or any of the exemplary pairs of affinity domains described herein), directly abuts the soluble tissue factor domain and/or the first domain of the pair of affinity domains. In some embodiments, the first chimeric polypeptide further comprises a linker sequence (e.g., any of the exemplary linker sequences described herein or known in the art) disposed (i) between the soluble tissue factor domain (e.g., any of the exemplary soluble tissue factor domains described herein) and the at least one of the one or more additional target-binding domains (e.g., any of the exemplary target-binding domains described herein or known in the art) positioned between the soluble tissue factor domain (e.g., any of the exemplary soluble tissue factor domains described herein) and the first domain of the pair of affinity domains (e.g., any of the exemplary first domains of any of the exemplary pairs of affinity domains described herein), and/or (ii) between the first domain of the pair of affinity domains and the at least one of the one or more additional target-binding domains positioned between the soluble tissue factor domain and the first domain of the pair of affinity domains.

In some embodiments of any of the multi-chain chimeric polypeptides described herein, the second chimeric polypeptide further includes one or more (e.g., two, three, four, five, six, seven, eight, nine, or ten) additional target-binding domains (e.g., any of the exemplary target-binding domains described herein or known in the art) at the N-terminal end and/or the C-terminal end of the second chimeric polypeptide. In some embodiments, at least one of the one or more additional target-binding domains (e.g., any of the exemplary target-binding domains described herein or known in the art) directly abuts the second domain of the pair of affinity domains (e.g., any of the exemplary second domains of any of the exemplary pairs of affinity domains described herein) in the second chimeric polypeptide. In some embodiments, the second chimeric polypeptide further includes a linker sequence (e.g., any of the exemplary linker sequences described herein or known in the art) between at least one of the one or more additional target-binding domains (e.g., any of the exemplary target-binding domains described herein or known in the art) and the second domain of the pair of affinity domains (e.g., any of the second domains described herein of any of the exemplary pairs of affinity domains described herein) in the second chimeric polypeptide. In some embodiments, at least one of the one or more additional target-binding domains (e.g., any of the exemplary target-binding domains described herein or known in the art) directly abuts the second target-binding domain (e.g., any of the target-binding domains described herein or known in the art) in the second chimeric polypeptide. In some embodiments, the second chimeric polypeptide further includes a linker sequence (e.g., any of the exemplary linker sequences described herein or known in the art) between at least one of the one or more additional target-binding domains (e.g., any of the exemplary target binding domains described herein or known in the art) and the second target-binding domain (e.g., any of the exemplary target binding domains described herein or known in the art) in the second chimeric polypeptide.

In some embodiments of any of the multi-chain chimeric polypeptides described herein, two or more (e.g., three or more, four or more, five or more, six or more, seven or more, eight or more, nine or more, or ten or more) of the first target-binding domain, the second target-binding domain, and the one or more additional target-binding domains bind specifically to the same antigen. In some embodiments, two or more (e.g., three or more, four or more, five or more, six or more, seven or more, eight or more, nine or more, or ten or more) of the first target-binding domain, the second target-binding domain, and the one or more additional target-binding domains bind specifically to the same epitope. In some embodiments, two or more (e.g., three or more, four or more, five or more, six or more, seven or more, eight or more, nine or more, or ten or more) of the first target-binding domain, the second target-binding domain, and the one or more additional target-binding domains include the same amino acid sequence. In some embodiments, the first target-binding domain, the second target-binding domain, and the one or more additional target-binding domains each bind specifically to the same antigen. In some embodiments, the first target-binding domain, the second target-binding domain, and the one or more additional target-binding domains each bind specifically to the same epitope. In some embodiments, the first target-binding domain, the second target-binding domain, and the one or more additional target-binding domains each include the same amino acid sequence.

In some embodiments of any of the multi-chain chimeric polypeptides described herein, the first target-binding domain, the second target-binding domain, and the one or more additional target-binding domains bind specifically to different antigens. In some embodiments of any of the multi-chain chimeric polypeptides described herein, one or more (e.g., two or more, three or more, four or more, five or more, six or more, seven or more, eight or more, nine or more, or ten or more) of the first target-binding domain, the second target-binding domain, and the one or more target-binding domains is an antigen-binding domain. In some embodiments, the first target-binding domain, the second target-binding domain, and the one or more additional target-binding domains are each an antigen-binding domain (e.g., a scFv or a single-domain antibody).

### Pairs of Affinity Domains

In some embodiments, a multi-chain chimeric polypeptide includes: 1) a first chimeric polypeptide that includes a first domain of a pair of affinity domains, and 2) a second chimeric polypeptide that includes a second domain of a pair of affinity domains such that the first chimeric polypeptide and the second chimeric polypeptide associate through the binding of the first domain and the second domain of the pair of affinity domains. In some embodiments, the pair of affinity domains is a sushi domain from an alpha chain of human IL-15 receptor (IL15Rα) and a soluble IL-15. A sushi domain, also known as a short consensus repeat or type 1 glycoprotein motif, is a common motif in protein-protein interaction. Sushi domains have been identified on a number of protein-binding molecules, including complement components C1r, C1s, factor H, and C2m, as well as the nonimmunologic molecules factor XIII and β2-glycoprotein. A typical Sushi domain has approximately 60 amino acid residues and contains four cysteines (Ranganathan, Pac. Symp Biocomput. 2000:155-67). The first cysteine can form a disulfide bond with the third cysteine, and the second cysteine can form a disulfide bridge with the fourth cysteine. In some embodiments in which one member of the pair of affinity domains is a soluble IL-15, the soluble IL15 has a D8N or D8A amino acid substitution. In some embodiments in which one member of the pair of affinity domains is an alpha chain of human IL-15 receptor (IL15Rα), the human IL15Rα is a mature full-length IL15Rα. In some embodiments, the pair of affinity domains is barnase and barnstar. In some embodiments, the pair of affinity domains is a PKA and an AKAP. In some embodiments, the pair of affinity domains is an adapter/docking tag module based on mutated RNase I fragments (Rossi, Proc Natl Acad Sci USA. 103:6841-6846, 2006; Sharkey et al., Cancer Res. 68:5282-5290, 2008; Rossi et al., Trends Pharmacol Sci. 33:474-481, 2012) or SNARE modules based on interactions of the proteins syntaxin, synaptotagmin, synaptobrevin, and SNAP25 (Deyev et al., Nat Biotechnol. 1486-1492, 2003).

In some embodiments, a first chimeric polypeptide of a multi-chain chimeric polypeptide includes a first domain of a pair of affinity domains and a second chimeric polypeptide of the multi-chain chimeric polypeptide includes a second domain of a pair of affinity domains, wherein the first domain of the pair of affinity domains and the second domain of the pair of affinity domains bind to each other with a dissociation equilibrium constant (K_{D}) of less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M. In some embodiments, the first domain of the pair of affinity domains and the second domain of the pair of affinity domains bind to each other with a K_{D} of about 1×10⁻⁴ M to about 1×10⁻⁶ M, about 1×10⁻⁵ M to about 1×10⁻⁷ M, about 1×10⁻⁶ M to about 1×10⁻⁸ M, about 1×10⁻⁷ M to about 1×10⁻⁹ M, about 1×10⁻⁸ M to about 1×10⁻¹⁰ M, about 1×10⁻⁹ M to about 1×10⁻¹¹ M, about 1×10⁻¹⁰ M to about 1×10⁻¹² M, about 1×10⁻¹¹ M to about 1×10⁻¹³ M, about 1×10⁻⁴ M to about 1×10⁻⁵ M, about 1×10⁻⁵ M to about 1×10⁻⁶ M, about 1×10⁻⁶ M to about 1×10⁻⁷ M, about 1×10⁻⁷ M to about 1×10⁻⁸ M, about 1×10⁻⁸ M to about 1×10⁻⁹ M, about 1×10⁻⁹ M to about 1×10⁻¹⁰ M, about 1×10⁻¹⁰ M to about 1×10⁻¹¹ M, about 1×10⁻¹¹ M to about 1×10⁻¹² M, or about 1×10⁻¹² M to about 1×10⁻¹³ M (inclusive). Any of a variety of different methods known in the art can be used to determine the K_{D} value of the binding of the first domain of the pair of affinity domains and the second domain of the pair of affinity domains (e.g., an electrophoretic mobility shift assay, a filter binding assay, surface plasmon resonance, and a biomolecular binding kinetics assay, etc.).

In some embodiments, a first chimeric polypeptide of a multi-chain chimeric polypeptide includes a first domain of a pair of affinity domains and a second chimeric polypeptide of the multi-chain chimeric polypeptide includes a second domain of a pair of affinity domains, wherein the first domain of the pair of affinity domains, the second domain of the pair of affinity domains, or both is about 10 to 100 amino acids in length. For example, a first domain of a pair of affinity domains, a second domain of a pair of affinity domains, or both can be about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about 10 to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a first domain of a pair of affinity domains, a second domain of a pair of affinity domains, or both is about 10, 15, 20, 25, 30, 35, 40, 45, 50, 55, 60, 65, 70, 75, 80, 85, 90, 95, or 100 amino acids in length.

In some embodiments, any of the first and/or second domains of a pair of affinity domains disclosed herein can include one or more additional amino acids (e.g., 1, 2, 3, 5, 6, 7, 8, 9, 10, or more amino acids) at its N-terminus and/or C-terminus, so long as the function of the first and/or second domains of a pair of affinity domains remains intact. For example, a sushi domain from an alpha chain of human IL-15 receptor (IL15Rα) can include one or more additional amino acids at the N-terminus and/or the C-terminus, while still retaining the ability to bind to a soluble IL-15. Additionally or alternatively, a soluble IL-15 can include one or more additional amino acids at the N-terminus and/or the C-terminus, while still retaining the ability to bind to a sushi domain from an alpha chain of human IL-15 receptor (IL15Rα).

A non-limiting example of a sushi domain from an alpha chain of IL-15 receptor alpha (IL15Rα) can include a sequence that is at least 70% identical, at least 75% identical, at least 80% identical, at least 85% identical, at least 90% identical, at least 95% identical, at least 99% identical, or 100% identical to ITCPPPMSVEHADIWVKSYSLYSRERYICNSGFKRKAGTSSLTECVLNKATNVAH WTTPSLKCIR (SEQ ID NO: 29). In some embodiments, a sushi domain from an alpha chain of IL15Rα can be encoded by a nucleic acid including

(SEQ ID NO: 30)
ATTACATGCCCCCCTCCCATGAGCGTGGAGCACGCCGACATCTGGGTGA
AGAGCTATAGCCTCTACAGCCGGGAGAGGTATATCTGTAACAGCGGCTT
CAAGAGGAAGGCCGGCACCAGCAGCCTCACCGAGTGCGTGCTGAATAAG
GCTACCAACGTGGCTCACTGGACAACACCCTCTTTAAAGTGCATCCGG.

In some embodiments, a soluble IL-15 can include a sequence that is at least 70% identical, at least 75% identical, at least 80% identical, at least 85% identical, at least 90% identical, at least 95% identical, at least 99% identical, or 100% identical to NWVNVISDLKKIEDLIQSMHIDATLYTESDVHPSCKVTAMKCFLLELQVISLESGD ASIHDTVENLIILANNSLSSNGNVTESGCKECEELEEKNIKEFLQSFVHIVQMFINT S (SEQ ID NO: 15). In some embodiments, a soluble IL-15 can be encoded by a nucleic acid including the sequence of

(SEQ ID NO: 31)
AACTGGGTGAACGTCATCAGCGATTTAAAGAAGATCGAAGATTTAATTC
AGTCCATGCATATCGACGCCACTTTATACACAGAATCCGACGTGCACCC
CTCTTGTAAGGTGACCGCCATGAAATGTTTTTTACTGGAGCTGCAAGTT
ATCTCTTTAGAGAGCGGAGACGCTAGCATCCACGACACCGTGGAGAATT
TAATCATTTTAGCCAATAACTCTTTATCCAGCAACGGCAACGTGACAGA
GTCCGGCTGCAAGGAGTGCGAAGAGCTGGAGGAGAAGAACATCAAGGAG
TTTCTGCAATCCTTTGTGCACATTGTCCAGATGTTCATCAATACCTCC.

### Signal Sequence

In some embodiments, a multi-chain chimeric polypeptide includes a first chimeric polypeptide that includes a signal sequence at its N-terminal end. In some embodiments, a multi-chain chimeric polypeptide includes a second chimeric polypeptide that includes a signal sequence at its N-terminal end. In some embodiments, both the first chimeric polypeptide of a multi-chain chimeric polypeptide and a second chimeric polypeptide of the multi-chain chimeric polypeptide include a signal sequence. As will be understood by those of ordinary skill in the art, a signal sequence is an amino acid sequence that is present at the N-terminus of a number of endogenously produced proteins that directs the protein to the secretory pathway (e.g., the protein is directed to reside in certain intracellular organelles, to reside in the cell membrane, or to be secreted from the cell). Signal sequences are heterogeneous and differ greatly in their primary amino acid sequences. However, signal sequences are typically 16 to 30 amino acids in length and include a hydrophilic, usually positively charged N-terminal region, a central hydrophobic domain, and a C-terminal region that contains the cleavage site for signal peptidase.

In some embodiments, a first chimeric polypeptide of a multi-chain chimeric polypeptide, a second chimeric polypeptide of the multi-chain chimeric polypeptide, or both includes a signal sequence having an amino acid sequence MKWVTFISLLFLFSSAYS (SEQ ID NO: 32). In some embodiments, a first chimeric polypeptide of a multi-chain chimeric polypeptide, a second chimeric polypeptide of the multi-chain chimeric polypeptide, or both includes a signal sequence encoded by the nucleic acid sequence

(SEQ ID NO: 33)
ATGAAATGGGTGACCTTTATTTCTTTACTGTTCCTCTTTAGCAGCGCCT
ACTCC,
(SEQ ID NO: 34)
ATGAAGTGGGTCACATTTATCTCTTTACTGTTCCTCTTCTCCAGCGCCT
ACAGC,
or
(SEQ ID NO: 35)
ATGAAATGGGTGACCTTTATTTCTTTACTGTTCCTCTTTAGCAGCGCCT
ACTCC.

In some embodiments, a first chimeric polypeptide of a multi-chain chimeric polypeptide, a second chimeric polypeptide of the multi-chain chimeric polypeptide, or both includes a signal sequence having an amino acid sequence MKCLLYLAFLFLGVNC (SEQ ID NO: 36). In some embodiments, a first chimeric polypeptide of a multi-chain chimeric polypeptide, a second chimeric polypeptide of the multi-chain chimeric polypeptide, or both includes a signal sequence having an amino acid sequence MGQIVTMFEALPHIIDEVINIVIIVLIIITSIKAVYNFATCGILALVSFLFLAGRSCG (SEQ ID NO: 37). In some embodiments, a first chimeric polypeptide of a multi-chain chimeric polypeptide, a second chimeric polypeptide of the multi-chain chimeric polypeptide, or both includes a signal sequence having an amino acid sequence

(SEQ ID NO: 38)
MPNHQSGSPTGSSDLLLSGKKQRPHLALRRKRRREMRKINRKVRRMNLA
PIKEKTAWQHLQALISEAEEVLKTSQTPQNSLTLFLALLSVLGPPVTG.

In some embodiments, a first chimeric polypeptide of a multi-chain chimeric polypeptide, a second chimeric polypeptide of the multi-chain chimeric polypeptide, or both includes a signal sequence having an amino acid sequence MDSKGSSQKGSRLLLLLVVSNLLLCQGVVS (SEQ ID NO: 39). Those of ordinary skill in the art will be aware of other appropriate signal sequences for use in a first chimeric polypeptide and/or a second chimeric polypeptide of multi-chain chimeric polypeptides described herein.

In some embodiments, a first chimeric polypeptide of a multi-chain chimeric polypeptide, a second chimeric polypeptide of the multi-chain chimeric polypeptide, or both includes a signal sequence that is about 10 to 100 amino acids in length. For example, a signal sequence can be about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about 10 to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a signal sequence is about 10, 15, 20, 25, 30, 35, 40, 45, 50, 55, 60, 65, 70, 75, 80, 85, 90, 95, or 100 amino acids in length.

In some embodiments, any of the signal sequences disclosed herein can include one or more additional amino acids (e.g., 1, 2, 3, 5, 6, 7, 8, 9, 10, or more amino acids) at its N-terminus and/or C-terminus, so long as the function of the signal sequence remains intact. For example, a signal sequence having the amino acid sequence MKCLLYLAFLFLGVNC (SEQ ID NO: 36) can include one or more additional amino acids at the N-terminus or C-terminus, while still retaining the ability to direct a first chimeric polypeptide of a multi-chain chimeric polypeptide, a second chimeric polypeptide of the multi-chain chimeric polypeptide, or both to the secretory pathway.

In some embodiments, a first chimeric polypeptide of a multi-chain chimeric polypeptide, a second chimeric polypeptide of the multi-chain chimeric polypeptide, or both includes a signal sequence that directs the multi-chain chimeric polypeptide into the extracellular space. Such embodiments are useful in producing multi-chain chimeric polypeptides that are relatively easy to be isolated and/or purified.

### Peptide Tags

In some embodiments, a multi-chain chimeric polypeptide includes a first chimeric polypeptide that includes a peptide tag (e.g., at the N-terminal end or the C-terminal end of the first chimeric polypeptide). In some embodiments, a multi-chain chimeric polypeptide includes a second chimeric polypeptide that includes a peptide tag (e.g., at the N-terminal end or the C-terminal end of the second chimeric polypeptide). In some embodiments, both the first chimeric polypeptide of a multi-chain chimeric polypeptide and a second chimeric polypeptide of the multi-chain chimeric polypeptide include a peptide tag. In some embodiments, a first chimeric polypeptide of a multi-chain chimeric polypeptide, a second chimeric polypeptide of the multi-chain chimeric polypeptide, or both include two or more peptide tags.

Exemplary peptide tags that can be included in a first chimeric polypeptide of a multi-chain chimeric polypeptide, a second chimeric polypeptide of the multi-chain chimeric polypeptide, or both include, without limitation, AviTag (GLNDIFEAQKIEWHE; SEQ ID NO: 40), a calmodulin-tag (KRRWKKNFIAVSAANRFKKISSSGAL; SEQ ID NO: 41), a polyglutamate tag (EEEEEE; SEQ ID NO: 42), an E-tag (GAPVPYPDPLEPR; SEQ ID NO: 43), a FLAG-tag (DYKDDDDK; SEQ ID NO: 44), an HA-tag, a peptide from hemagglutinin (YPYDVPDYA; SEQ ID NO: 45), a his-tag (HHHHH (SEQ ID NO: 46); HHHHHH (SEQ ID NO: 47); HHHHHHH (SEQ ID NO: 48); HHHHHHHH (SEQ ID NO: 49); HHHHHHHHH (SEQ ID NO: 50); or HHHHHHHHHH (SEQ ID NO: 51), a myc-tag (EQKLISEEDL; SEQ ID NO: 52), NE-tag (TKENPRSNQEESYDDNES; SEQ ID NO: 53), S-tag, (KETAAAKFERQHMDS; SEQ ID NO: 54), SBP-tag (MDEKTTGWRGGHVVEGLAGELEQLRARLEHHPQGQREP; SEQ ID NO: 55), Softag 1 (SLAELLNAGLGGS; SEQ ID NO: 56), Softag 3 (TQDPSRVG; SEQ ID NO: 57), Spot-tag (PDRVRAVSHWSS; SEQ ID NO: 58), Strep-tag (WSHPQFEK; SEQ ID NO: 59), TC tag (CCPGCC; SEQ ID NO: 60), Ty tag (EVHTNQDPLD; SEQ ID NO: 61), V5 tag (GKPIPNPLLGLDST; SEQ ID NO: 62), VSV-tag (YTDIEMNRLGK; SEQ ID NO: 63), and Xpress tag (DLYDDDDK; SEQ ID NO: 64). In some embodiments, tissue factor protein is a peptide tag.

Peptide tags that can be included in a first chimeric polypeptide of a multi-chain chimeric polypeptide, a second chimeric polypeptide of the multi-chain chimeric polypeptide, or both can be used in any of a variety of applications related to the multi-chain chimeric polypeptide. For example, a peptide tag can be used in the purification of a multi-chain chimeric polypeptide. As one non-limiting example, a first chimeric polypeptide of a multi-chain chimeric polypeptide (e.g., a recombinantly expressed first chimeric polypeptide), a second chimeric polypeptide of the multi-chain chimeric polypeptide (e.g., a recombinantly expressed second chimeric polypeptide), or both can include a myc tag; the multi-chain chimeric polypeptide that includes the myc-tagged first chimeric polypeptide, the myc-tagged second chimeric polypeptide, or both can be purified using an antibody that recognizes the myc tag(s). One non-limiting example of an antibody that recognizes a myc tag is 9E10, available from the non-commercial Developmental Studies Hybridoma Bank. As another non-limiting example, a first chimeric polypeptide of a multi-chain chimeric polypeptide (e.g., a recombinantly expressed first chimeric polypeptide), a second chimeric polypeptide of the multi-chain chimeric polypeptide (e.g., a recombinantly expressed second chimeric polypeptide), or both can include a histidine tag; the multi-chain chimeric polypeptide that includes the histidine-tagged first chimeric polypeptide, the histidine-tagged second chimeric polypeptide, or both can be purified using a nickel or cobalt chelate. Those of ordinary skill in the art will be aware of other suitable tags and agent that bind those tags for use in purifying multi-chain chimeric polypeptide. In some embodiments, a peptide tag is removed from the first chimeric polypeptide and/or the second chimeric polypeptide of the multi-chain chimeric polypeptide after purification. In some embodiments, a peptide tag is not removed from the first chimeric polypeptide and/or the second chimeric polypeptide of the multi-chain chimeric polypeptide after purification.

Peptide tags that can be included in a first chimeric polypeptide of a multi-chain chimeric polypeptide, a second chimeric polypeptide of the multi-chain chimeric polypeptide, or both can be used, for example, in immunoprecipitation of the multi-chain chimeric polypeptide, imaging of the multi-chain chimeric polypeptide (e.g., via Western blotting, ELISA, flow cytometry, and/or immunocytochemistry), and/or solubilization of the multi-chain chimeric polypeptide.

In some embodiments, a first chimeric polypeptide of a multi-chain chimeric polypeptide, a second chimeric polypeptide of the multi-chain chimeric polypeptide, or both includes a peptide tag that is about 10 to 100 amino acids in length. For example, a peptide tag can be about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a peptide tag is about 10, 15, 20, 25, 30, 35, 40, 45, 50, 55, 60, 65, 70, 75, 80, 85, 90, 95, or 100 amino acids in length.

Peptide tags included in a first chimeric polypeptide of a multi-chain chimeric polypeptide, a second chimeric polypeptide of the multi-chain chimeric polypeptide, or both can be of any suitable length. For example, peptide tags can be 5, 6, 7, 8, 9, 10, 11, 12, 13, 14, 15, 16, 17, 18, 19, 20, or more amino acids in length. In embodiments in which a multi-chain chimeric polypeptide includes two or more peptide tags, the two or more peptide tags can be of the same or different lengths. In some embodiments, any of the peptide tags disclosed herein may include one or more additional amino acids (e.g., 1, 2, 3, 5, 6, 7, 8, 9, 10, or more amino acids) at the N-terminus and/or C-terminus, so long as the function of the peptide tag remains intact. For example, a myc tag having the amino acid sequence EQKLISEEDL (SEQ ID NO: 65) can include one or more additional amino acids (e.g., at the N-terminus and/or the C-terminus of the peptide tag), while still retaining the ability to be bound by an antibody (e.g., 9E10).

### Exemplary Multi-Chain Chimeric Polypeptides

In some examples of the multi-chain chimeric polypeptides, the first target-binding domain and the soluble tissue factor domain directly abut each other in the first chimeric polypeptide. In some embodiments of the multi-chain chimeric polypeptides, the soluble tissue factor domain and the first domain of the pair of affinity domains directly abut each other in the first chimeric polypeptide.

In some embodiments of these multi-chain chimeric polypeptides, the second domain of the pair of affinity domains and the second target-binding domain directly abut each other in the second chimeric polypeptide.

In some embodiments of these multi-chain chimeric polypeptides, the soluble tissue factor domain can be any of the exemplary soluble tissue factor domains described herein. In some embodiments of these multi-chain chimeric polypeptides, the pair of affinity domains can be any of the exemplary pairs of affinity domains described herein.

In some embodiments of these multi-chain chimeric polypeptides, one or both of the first target-binding domain and the second target-binding domain is a soluble TGF-β receptor (e.g., a soluble TGFRβRII, e.g., a soluble human TGFRβRII).

In some embodiments of the multi-chain chimeric polypeptides, the soluble human TGFRβRII includes a first sequence of soluble human TGFRβRII and a second sequence of soluble human TGFRβRII. In some embodiments of these multi-chain chimeric polypeptides, the soluble human TGFRβRII includes a linker disposed between the first sequence of soluble human TGFRβRII and the second sequence of soluble human TGFRβRII. In some examples of these multi-chain chimeric polypeptides, the linker includes the sequence GGGGSGGGGSGGGGS (SEQ ID NO: 7).

In some embodiments of these multi-chain chimeric polypeptides, the first sequence of soluble human TGFRβRII receptor comprises a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 66)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPD.

In some embodiments of these multi-chain chimeric polypeptides, the second sequence of soluble human TGFRβRII receptor comprises a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 66)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPD.

In some embodiments of these multi-chain chimeric polypeptides, the first sequence of soluble human TGFRβRII receptor is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 67)
ATTCCTCCCCACGTGCAGAAGAGCGTGAATAATGACATGATCGTGACCG
ATAACAATGGCGCCGTGAAATTTCCCCAGCTGTGCAAATTCTGCGATGT
GAGGTTTTCCACCTGCGACAACCAGAAGTCCTGTATGAGCAACTGCACA
ATCACCTCCATCTGTGAGAAGCCTCAGGAGGTGTGCGTGGCTGTCTGGC
GGAAGAATGACGAGAATATCACCCTGGAAACCGTCTGCCACGATCCCAA
GCTGCCCTACCACGATTTCATCCTGGAAGACGCCGCCAGCCCTAAGTGC
ATCATGAAAGAGAAAAAGAAGCCTGGCGAGACCTTTTTCATGTGCTCCT
GCAGCAGCGACGAATGCAACGACAATATCATCTTTAGCGAGGAATACAA
TACCAGCAACCCCGAC.

In some embodiments of these multi-chain chimeric polypeptides, the second sequence of soluble human TGFRβRII receptor is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 67)
ATTCCTCCCCACGTGCAGAAGAGCGTGAATAATGACATGATCGTGACCG
ATAACAATGGCGCCGTGAAATTTCCCCAGCTGTGCAAATTCTGCGATGT
GAGGTTTTCCACCTGCGACAACCAGAAGTCCTGTATGAGCAACTGCACA
ATCACCTCCATCTGTGAGAAGCCTCAGGAGGTGTGCGTGGCTGTCTGGC
GGAAGAATGACGAGAATATCACCCTGGAAACCGTCTGCCACGATCCCAA
GCTGCCCTACCACGATTTCATCCTGGAAGACGCCGCCAGCCCTAAGTGC
ATCATGAAAGAGAAAAAGAAGCCTGGCGAGACCTTTTTCATGTGCTCCT
GCAGCAGCGACGAATGCAACGACAATATCATCTTTAGCGAGGAATACAA
TACCAGCAACCCCGAC.

In some embodiments of these multi-chain chimeric polypeptides, the soluble human TGFRβRII receptor is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 68)
ATCCCCCCCCATGTGCAAAAGAGCGTGAACAACGATATGATCGTGACCG
ACAACAACGGCGCCGTGAAGTTTCCCCAGCTCTGCAAGTTCTGCGATGT
CAGGTTCAGCACCTGCGATAATCAGAAGTCCTGCATGTCCAACTGCACG
ATCACCTCCATCTGCGAGAAGCCCCAAGAAGTGTGCGTGGCCGTGTGGC
GGAAAAATGACGAGAACATCACCCTGGAGACCGTGTGTCACGACCCCAA
GCTCCCTTATCACGACTTCATTCTGGAGGACGCTGCCTCCCCCAAATGC
ATCATGAAGGAGAAGAAGAAGCCCGGAGAGACCTTCTTTATGTGTTCCT
GTAGCAGCGACGAGTGTAACGACAACATCATCTTCAGCGAAGAGTACAA
CACCAGCAACCCTGATGGAGGTGGCGGATCCGGAGGTGGAGGTTCTGGT
GGAGGTGGGAGTATTCCTCCCCACGTGCAGAAGAGCGTGAATAATGACA
TGATCGTGACCGATAACAATGGCGCCGTGAAATTTCCCCAGCTGTGCAA
ATTCTGCGATGTGAGGTTTTCCACCTGCGACAACCAGAAGTCCTGTATG
AGCAACTGCACAATCACCTCCATCTGTGAGAAGCCTCAGGAGGTGTGCG
TGGCTGTCTGGCGGAAGAATGACGAGAATATCACCCTGGAAACCGTCTG
CCACGATCCCAAGCTGCCCTACCACGATTTCATCCTGGAAGACGCCGCC
AGCCCTAAGTGCATCATGAAAGAGAAAAAGAAGCCTGGCGAGACCTTTT
TCATGTGCTCCTGCAGCAGCGACGAATGCAACGACAATATCATCTTTAG
CGAGGAATACAATACCAGCAACCCCGAC.

In some embodiments of these multi-chain chimeric polypeptides, the human TGFβRII receptor includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 69)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPD.

In some embodiments, the first chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 70)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPDSGTTNTV
AAYNLTWKSTNFKTILEWEPKPVNQVYTVQISTKSGDWKSKCFYTTDTE
CDLTDEIVKDVKQTYLARVFSYPAGNVESTGSAGEPLYENSPEFTPYLE
TNLGQPTIQSFEQVGTKVNVTVEDERTLVRRNNTFLSLRDVFGKDLIYT
LYYWKSSSSGKKTAKTNTNEFLIDVDKGENYCFSVQAVIPSRTVNRKST
DSPVECMGQEKGEFRENWVNVISDLKKIEDLIQSMHIDATLYTESDVHP
SCKVTAMKCFLLELQVISLESGDASIHDTVENLIILANNSLSSNGNVTE
SGCKECEELEEKNIKEFLQSFVHIVQMFINTS.

In some embodiments, a first chimeric polypeptide is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 71)
ATCCCCCCCCATGTGCAAAAGAGCGTGAACAACGATATGATCGTGACCG
ACAACAACGGCGCCGTGAAGTTTCCCCAGCTCTGCAAGTTCTGCGATGT
CAGGTTCAGCACCTGCGATAATCAGAAGTCCTGCATGTCCAACTGCAGC
ATCACCTCCATCTGCGAGAAGCCCCAAGAAGTGTGCGTGGCCGTGTGGC
GGAAAAATGACGAGAACATCACCCTGGAGACCGTGTGTCACGACCCCAA
GCTCCCTTATCACGACTTCATTCTGGAGGACGCTGCCTCCCCCAAATGC
ATCATGAAGGAGAAGAAGAAGCCCGGAGAGACCTTCTTTATGTGTTCCT
GTAGCAGCGACGAGTGTAACGACAACATCATCTTCAGCGAAGAGTACAA
CACCAGCAACCCTGATGGAGGTGGCGGATCCGGAGGTGGAGGTTCTGGT
GGAGGTGGGAGTATTCCTCCCCACGTGCAGAAGAGCGTGAATAATGACA
TGATCGTGACCGATAACAATGGCGCCGTGAAATTTCCCCAGCTGTGCAA
ATTCTGCGATGTGAGGTTTTCCACCTGCGACAACCAGAAGTCCTGTATG
AGCAACTGCTCCATCACCTCCATCTGTGAGAAGCCTCAGGAGGTGTGCG
TGGCTGTCTGGCGGAAGAATGACGAGAATATCACCCTGGAAACCGTCTG
CCACGATCCCAAGCTGCCCTACCACGATTTCATCCTGGAAGACGCCGCC
AGCCCTAAGTGCATCATGAAAGAGAAAAAGAAGCCTGGCGAGACCTTTT
TCATGTGCTCCTGCAGCAGCGACGAATGCAACGACAATATCATCTTTAG
CGAGGAATACAATACCAGCAACCCCGACAGCGGCACAACCAACACAGTC
GCTGCCTATAACCTCACTTGGAAGAGCACCAACTTCAAAACCATCCTCG
AATGGGAACCCAAACCCGTTAACCAAGTTTACACCGTGCAGATCAGCAC
CAAGTCCGGCGACTGGAAGTCCAAATGTTTCTATACCACCGACACCGAG
TGCGATCTCACCGATGAGATCGTGAAAGATGTGAAACAGACCTACCTCG
CCCGGGTGTTTAGCTACCCCGCCGGCAATGTGGAGAGCACTGGTTCCGC
TGGCGAGCCTTTATACGAGAACAGCCCCGAATTTACCCCTTACCTCGAG
ACCAATTTAGGACAGCCCACCATCCAAAGCTTTGAGCAAGTTGGCACAA
AGGTGAATGTGACAGTGGAGGACGAGCGGACTTTAGTGCGGCGGAACAA
CACCTTTCTCAGCCTCCGGGATGTGTTCGGCAAAGATTTAATCTACACA
CTGTATTACTGGAAGTCCTCTTCCTCCGGCAAGAAGACAGCTAAAACCA
ACACAAACGAGTTTTTAATCGACGTGGATAAAGGCGAAAACTACTGTTT
CAGCGTGCAAGCTGTGATCCCCTCCCGGACCGTGAATAGGAAAAGCACC
GATAGCCCCGTTGAGTGCATGGGCCAAGAAAAGGGCGAGTTCCGGGAGA
ACTGGGTGAACGTCATCAGCGATTTAAAGAAGATCGAAGATTTAATTCA
GTCCATGCATATCGACGCCACTTTATACACAGAATCCGACGTGCACCCC
TCTTGTAAGGTGACCGCCATGAAATGTTTTTTACTGGAGCTGCAAGTTA
TCTCTTTAGAGAGCGGAGACGCTAGCATCCACGACACCGTGGAGAATTT
AATCATTTTAGCCAATAACTCTTTATCCAGCAACGGCAACGTGACAGAG
TCCGGCTGCAAGGAGTGCGAAGAGCTGGAGGAGAAGAACATCAAGGAGT
TTCTGCAATCCTTTGTGCACATTGTCCAGATGTTCATCAATACCTCC.

In some embodiments, a first chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 72)
MKWVTFISLLFLFSSAYSIPPHVQKSVNNDMIVTDNNGAVKFPQLCKFC
DVRFSTCDNQKSCMSNCSITSICEKPQEVCVAVWRKNDENITLETVCHD
PKLPYHDFILEDAASPKCIMKEKKKPGETFFMCSCSSDECNDNIIFSEE
YNTSNPDGGGGSGGGGSGGGGSIPPHVQKSVNNDMIVTDNNGAVKFPQL
CKFCDVRFSTCDNQKSCMSNCSITSICEKPQEVCVAVWRKNDENITLET
VCHDPKLPYHDFILEDAASPKCIMKEKKKPGETFFMCSCSSDECNDNII
FSEEYNTSNPDSGTTNTVAAYNLTWKSTNFKTILEWEPKPVNQVYTVQI
STKSGDWKSKCFYTTDTECDLTDEIVKDVKQTYLARVFSYPAGNVESTG
SAGEPLYENSPEFTPYLETNLGQPTIQSFEQVGTKVNVTVEDERTLVRR
NNTFLSLRDVFGKDLIYTLYYWKSSSSGKKTAKTNTNEFLIDVDKGENY
CFSVQAVIPSRTVNRKSTDSPVECMGQEKGEFRENWVNVISDLKKIEDL
IQSMHIDATLYTESDVHPSCKVTAMKCFLLELQVISLESGDASIHDTVE
NLIILANNSLSSNGNVTESGCKECEELEEKNIKEFLQSFVHIVQMFINT
S.

In some embodiments, a first chimeric polypeptide is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 73)
ATGAAGTGGGTGACCTTCATCAGCCTGCTGTTCCTGTTCTCCAGCGCCT
ACTCCATCCCCCCCCATGTGCAAAAGAGCGTGAACAACGATATGATCGT
GACCGACAACAACGGCGCCGTGAAGTTTCCCCAGCTCTGCAAGTTCTGC
GATGTCAGGTTCAGCACCTGCGATAATCAGAAGTCCTGCATGTCCAACT
GCAGCATCACCTCCATCTGCGAGAAGCCCCAAGAAGTGTGCGTGGCCGT
GTGGCGGAAAAATGACGAGAACATCACCCTGGAGACCGTGTGTCACGAC
CCCAAGCTCCCTTATCACGACTTCATTCTGGAGGACGCTGCCTCCCCCA
AATGCATCATGAAGGAGAAGAAGAAGCCCGGAGAGACCTTCTTTATGTG
TTCCTGTAGCAGCGACGAGTGTAACGACAACATCATCTTCAGCGAAGAG
TACAACACCAGCAACCCTGATGGAGGTGGCGGATCCGGAGGTGGAGGTT
CTGGTGGAGGTGGGAGTATTCCTCCCCACGTGCAGAAGAGCGTGAATAA
TGACATGATCGTGACCGATAACAATGGCGCCGTGAAATTTCCCCAGCTG
TGCAAATTCTGCGATGTGAGGTTTTCCACCTGCGACAACCAGAAGTCCT
GTATGAGCAACTGCTCCATCACCTCCATCTGTGAGAAGCCTCAGGAGGT
GTGCGTGGCTGTCTGGCGGAAGAATGACGAGAATATCACCCTGGAAACC
GTCTGCCACGATCCCAAGCTGCCCTACCACGATTTCATCCTGGAAGACG
CCGCCAGCCCTAAGTGCATCATGAAAGAGAAAAAGAAGCCTGGCGAGAC
CTTTTTCATGTGCTCCTGCAGCAGCGACGAATGCAACGACAATATCATC
TTTAGCGAGGAATACAATACCAGCAACCCCGACAGCGGCACAACCAACA
CAGTCGCTGCCTATAACCTCACTTGGAAGAGCACCAACTTCAAAACCAT
CCTCGAATGGGAACCCAAACCCGTTAACCAAGTTTACACCGTGCAGATC
AGCACCAAGTCCGGCGACTGGAAGTCCAAATGTTTCTATACCACCGACA
CCGAGTGCGATCTCACCGATGAGATCGTGAAAGATGTGAAACAGACCTA
CCTCGCCCGGGTGTTTAGCTACCCCGCCGGCAATGTGGAGAGCACTGGT
TCCGCTGGCGAGCCTTTATACGAGAACAGCCCCGAATTTACCCCTTACC
TCGAGACCAATTTAGGACAGCCCACCATCCAAAGCTTTGAGCAAGTTGG
CACAAAGGTGAATGTGACAGTGGAGGACGAGCGGACTTTAGTGCGGCGG
AACAACACCTTTCTCAGCCTCCGGGATGTGTTCGGCAAAGATTTAATCT
ACACACTGTATTACTGGAAGTCCTCTTCCTCCGGCAAGAAGACAGCTAA
AACCAACACAAACGAGTTTTTAATCGACGTGGATAAAGGCGAAAACTAC
TGTTTCAGCGTGCAAGCTGTGATCCCCTCCCGGACCGTGAATAGGAAAA
GCACCGATAGCCCCGTTGAGTGCATGGGCCAAGAAAAGGGCGAGTTCCG
GGAGAACTGGGTGAACGTCATCAGCGATTTAAAGAAGATCGAAGATTTA
ATTCAGTCCATGCATATCGACGCCACTTTATACACAGAATCCGACGTGC
ACCCCTCTTGTAAGGTGACCGCCATGAAATGTTTTTTACTGGAGCTGCA
AGTTATCTCTTTAGAGAGCGGAGACGCTAGCATCCACGACACCGTGGAG
AATTTAATCATTTTAGCCAATAACTCTTTATCCAGCAACGGCAACGTGA
CAGAGTCCGGCTGCAAGGAGTGCGAAGAGCTGGAGGAGAAGAACATCAA
GGAGTTTCTGCAATCCTTTGTGCACATTGTCCAGATGTTCATCAATACC
TCC.

In some embodiments, the second chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 74)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPDITCPPPM
SVEHADIWVKSYSLYSRERYICNSGFKRKAGTSSLTECVLNKATNVAHW
TTPSLKCIR.

In some embodiments, a second chimeric polypeptide is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 75)
ATCCCCCCCCATGTGCAAAAGAGCGTGAACAACGATATGATCGTGACCG
ACAACAACGGCGCCGTGAAGTTTCCCCAGCTCTGCAAGTTCTGCGATGT
CAGGTTCAGCACCTGCGATAATCAGAAGTCCTGCATGTCCAACTGCAGC
ATCACCTCCATCTGCGAGAAGCCCCAAGAAGTGTGCGTGGCCGTGTGGC
GGAAAAATGACGAGAACATCACCCTGGAGACCGTGTGTCACGACCCCAA
GCTCCCTTATCACGACTTCATTCTGGAGGACGCTGCCTCCCCCAAATGC
ATCATGAAGGAGAAGAAGAAGCCCGGAGAGACCTTCTTTATGTGTTCCT
GTAGCAGCGACGAGTGTAACGACAACATCATCTTCAGCGAAGAGTACAA
CACCAGCAACCCTGATGGAGGTGGCGGATCCGGAGGTGGAGGTTCTGGT
GGAGGTGGGAGTATTCCTCCCCACGTGCAGAAGAGCGTGAATAATGACA
TGATCGTGACCGATAACAATGGCGCCGTGAAATTTCCCCAGCTGTGCAA
ATTCTGCGATGTGAGGTTTTCCACCTGCGACAACCAGAAGTCCTGTATG
AGCAACTGCTCCATCACCTCCATCTGTGAGAAGCCTCAGGAGGTGTGCG
TGGCTGTCTGGCGGAAGAATGACGAGAATATCACCCTGGAAACCGTCTG
CCACGATCCCAAGCTGCCCTACCACGATTTCATCCTGGAAGACGCCGCC
AGCCCTAAGTGCATCATGAAAGAGAAAAAGAAGCCTGGCGAGACCTTTT
TCATGTGCTCCTGCAGCAGCGACGAATGCAACGACAATATCATCTTTAG
CGAGGAATACAATACCAGCAACCCCGACATTACATGCCCCCCTCCCATG
AGCGTGGAGCACGCCGACATCTGGGTGAAGAGCTATAGCCTCTACAGCC
GGGAGAGGTATATCTGTAACAGCGGCTTCAAGAGGAAGGCCGGCACCAG
CAGCCTCACCGAGTGCGTGCTGAATAAGGCTACCAACGTGGCTCACTGG
ACAACACCCTCTTTAAAGTGCATCCGG.

In some embodiments, a second chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 76)
MKWVTFISLLFLFSSAYSIPPHVQKSVNNDMIVTDNNGAVKFPQLCKFC
DVRFSTCDNQKSCMSNCSITSICEKPQEVCVAVWRKNDENITLETVCHD
PKLPYHDFILEDAASPKCIMKEKKKPGETFFMCSCSSDECNDNIIFSEE
YNTSNPDGGGGSGGGGSGGGGSIPPHVQKSVNNDMIVTDNNGAVKFPQL
CKFCDVRFSTCDNQKSCMSNCSITSICEKPQEVCVAVWRKNDENITLET
VCHDPKLPYHDFILEDAASPKCIMKEKKKPGETFFMCSCSSDECNDNII
FSEEYNTSNPDITCPPPMSVEHADIWVKSYSLYSRERYICNSGFKRKAG
TSSLTECVLNKATNVAHWTTPSLKCIR.

In some embodiments, a second chimeric polypeptide is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 77)
ATGAAGTGGGTGACCTTCATCAGCCTGCTGTTCCTGTTCTCCAGCGCCT
ACTCCATCCCCCCCCATGTGCAAAAGAGCGTGAACAACGATATGATCGT
GACCGACAACAACGGCGCCGTGAAGTTTCCCCAGCTCTGCAAGTTCTGC
GATGTCAGGTTCAGCACCTGCGATAATCAGAAGTCCTGCATGTCCAACT
GCAGCATCACCTCCATCTGCGAGAAGCCCCAAGAAGTGTGCGTGGCCGT
GTGGCGGAAAAATGACGAGAACATCACCCTGGAGACCGTGTGTCACGAC
CCCAAGCTCCCTTATCACGACTTCATTCTGGAGGACGCTGCCTCCCCCA
AATGCATCATGAAGGAGAAGAAGAAGCCCGGAGAGACCTTCTTTATGTG
TTCCTGTAGCAGCGACGAGTGTAACGACAACATCATCTTCAGCGAAGAG
TACAACACCAGCAACCCTGATGGAGGTGGCGGATCCGGAGGTGGAGGTT
CTGGTGGAGGTGGGAGTATTCCTCCCCACGTGCAGAAGAGCGTGAATAA
TGACATGATCGTGACCGATAACAATGGCGCCGTGAAATTTCCCCAGCTG
TGCAAATTCTGCGATGTGAGGTTTTCCACCTGCGACAACCAGAAGTCCT
GTATGAGCAACTGCTCCATCACCTCCATCTGTGAGAAGCCTCAGGAGGT
GTGCGTGGCTGTCTGGCGGAAGAATGACGAGAATATCACCCTGGAAACC
GTCTGCCACGATCCCAAGCTGCCCTACCACGATTTCATCCTGGAAGACG
CCGCCAGCCCTAAGTGCATCATGAAAGAGAAAAAGAAGCCTGGCGAGAC
CTTTTTCATGTGCTCCTGCAGCAGCGACGAATGCAACGACAATATCATC
TTTAGCGAGGAATACAATACCAGCAACCCCGACATTACATGCCCCCCTC
CCATGAGCGTGGAGCACGCCGACATCTGGGTGAAGAGCTATAGCCTCTA
CAGCCGGGAGAGGTATATCTGTAACAGCGGCTTCAAGAGGAAGGCCGGC
ACCAGCAGCCTCACCGAGTGCGTGCTGAATAAGGCTACCAACGTGGCTC
ACTGGACAACACCCTCTTTAAAGTGCATCCGG.

### Nucleic Acids/Vectors

Also provided herein are nucleic acids that encode any of the multi-chain chimeric polypeptides described herein. In some embodiments, a first nucleic acid can encode the first chimeric polypeptide and a second nucleic acid can encode the second chimeric polypeptide. In some embodiments, a single nucleic acid can encode both the first chimeric polypeptide and the second chimeric polypeptide.

Also provided herein are vectors that include any of the nucleic acids encoding any of the multi-chain chimeric polypeptides described herein. In some embodiments, a first vector can include a nucleic acid encoding the first chimeric polypeptide and a second vector can include a nucleic acid encoding the second chimeric polypeptide. In some embodiments, a single vector can include a first nucleic acid encoding the first chimeric polypeptide and a second nucleic acid encoding the second chimeric polypeptide.

Any of the vectors described herein can be an expression vector. For example, an expression vector can include a promoter sequence operably linked to the sequence encoding the first chimeric polypeptide and the second chimeric polypeptide.

Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. A vector can, e.g., include sufficient cis-acting elements for expression; other elements for expression can be supplied by the host mammalian cell or in an in vitro expression system. Skilled practitioners will be capable of selecting suitable vectors and mammalian cells for making any of the multi-chain chimeric polypeptides described herein.

### Cells

Also provided herein are cells (e.g., any of the exemplary cells described herein or known in the art) comprising any of the nucleic acids described herein that encode any of the multi-chain chimeric polypeptides described herein (e.g., encoding both the first and second chimeric polypeptides). Also provided herein are cells (e.g., any of the exemplary cells described herein or known in the art) comprising any of the nucleic acids described herein that encode any of the first chimeric polypeptides described herein. Also provided are cells (e.g., any of the exemplary cells described herein or known in the art) comprising any of the nucleic acids described herein that encode any of the second chimeric polypeptides described herein.

Also provided herein are cells (e.g., any of the exemplary cells described herein or known in the art) that include any of the vectors described herein that encode any of the multi-chain chimeric polypeptides described herein (e.g., encoding both the first and second chimeric polypeptides). Also provided herein are cells (e.g., any of the exemplary cells described herein or known in the art) that include any of the vectors described herein that encode any of the first chimeric polypeptides described herein. Also provided herein are cells (e.g., any of the exemplary cells described herein or known in the art) that include any of the vectors described herein that encode any of the second chimeric polypeptides described herein).

In some embodiments of any of the methods described herein, the cell can be a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Non-limiting examples of mammalian cells include Chinese hamster ovary cells and human embryonic kidney cells (e.g., HEK293 cells).

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral and lentiviral transduction), and nanoparticle transfection.

### Methods of Producing Multi-Chain Chimeric Polypeptides

Also provided herein are methods of producing any of the multi-chain chimeric polypeptides described herein that include culturing any of the cells described herein in a culture medium under conditions sufficient to result in the production of the multi-chain chimeric polypeptide; and recovering the multi-chain chimeric polypeptide from the cell and/or the culture medium.

Also provided herein are method of producing any of the multi-chain chimeric polypeptides described herein that include: culturing any of cells described herein in a first culture medium under conditions sufficient to result in the production of the first chimeric polypeptide; recovering the first chimeric polypeptide from the cell and/or the first culture medium; culturing any of the cells described herein in a second culture medium under conditions sufficient to result in the production of the second chimeric polypeptide; recovering the second chimeric polypeptide from the cell and/or the second culture medium; and combining (e.g., mixing) the recovered first chimeric polypeptide and the recovered second chimeric polypeptide to form the multi-chain chimeric polypeptide (e.g., any of the multi-chain chimeric polypeptides described herein).

The recovery of the multi-chain chimeric polypeptide, the first chimeric polypeptide, or the second chimeric polypeptide from a cell (e.g., a eukaryotic cell) can be performed using techniques well-known in the art (e.g., ammonium sulfate precipitation, polyethylene glycol precipitation, ion-exchange chromatography (anion or cation), chromatography based on hydrophobic interaction, metal-affinity chromatography, ligand-affinity chromatography, and size exclusion chromatography).

Methods of culturing cells are well known in the art. Cells can be maintained in vitro under conditions that favor proliferation, differentiation and growth. Briefly, cells can be cultured by contacting a cell (e.g., any cell) with a cell culture medium that includes the necessary growth factors and supplements to support cell viability and growth.

Also provided herein are multi-chain chimeric polypeptides (e.g., any of the multi-chain chimeric polypeptides described herein), first chimeric polypeptides (e.g., any of the first chimeric polypeptides), or second chimeric polypeptides (e.g., any of the second chimeric polypeptides described herein) produced by any of the methods described herein.

### Methods of Treatment

Provided herein are methods of treating unresectable advanced/metastatic pancreatic cancer in a subject (e.g., any of the exemplary subjects described herein or known in the art) that include administering to the subject a therapeutically effective amount of a multi-chain chimeric polypeptide, wherein the multi-chain chimeric polypeptide comprises: (a) a first chimeric polypeptide comprising: (i) a first target-binding domain; (ii) a soluble tissue factor domain; and (iii) a first domain of a pair of affinity domains; and (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII. Any of the exemplary multi-chain chimeric polypeptides described herein can be used in these methods.

In some embodiments, the methods described herein can result in a decrease (e.g., at least a 1% decrease, at least a 5% decrease, at least a 10% decrease, at least a 15% decrease, at least a 20% decrease, at least a 25% decrease, at least a 30% decrease, at least a 35% decrease, at least a 40% decrease, at least a 45% decrease, at least a 50% decrease, at least a 55% decrease, at least a 60% decrease, at least a 65% decrease, at least a 70% decrease, at least a 75% decrease, at least a 80% decrease, at least a 85% decrease, at least a 90% decrease, at least a 95% decrease, or at least a 99% decrease, or about a 1% decrease to about a 99% decrease, about a 1% decrease to about a 95% decrease, about a 1% decrease to about a 90% decrease, about a 1% decrease to about a 85% decrease, about a 1% decrease to about a 80% decrease, about a 1% decrease to about a 75% decrease, about a 1% decrease to about a 70% decrease, about a 1% decrease to about a 65% decrease, about a 1% decrease to about a 60% decrease, about a 1% decrease to about a 55% decrease, about a 1% decrease to about a 50% decrease, about a 1% decrease to about a 45% decrease, about a 1% decrease to about a 40% decrease, about a 1% decrease to about a 35% decrease, about a 1% decrease to about a 30% decrease, about a 1% decrease to about a 25% decrease, about a 1% decrease to about a 20% decrease, about a 1% decrease to about a 15% decrease, about a 1% decrease to about a 10% decrease, about a 1% decrease to about a 5% decrease, about a 5% decrease to about a 99% decrease, about a 5% decrease to about a 95% decrease, about a 5% decrease to about a 90% decrease, about a 5% decrease to about a 85% decrease, about a 5% decrease to about a 80% decrease, about a 5% decrease to about a 75% decrease, about a 5% decrease to about a 70% decrease, about a 5% decrease to about a 65% decrease, about a 5% decrease to about a 60% decrease, about a 5% decrease to about a 55% decrease, about a 5% decrease to about a 50% decrease, about a 5% decrease to about a 45% decrease, about a 5% decrease to about a 40% decrease, about a 5% decrease to about a 35% decrease, about a 5% decrease to about a 30% decrease, about a 5% decrease to about a 25% decrease, about a 5% decrease to about a 20% decrease, about a 5% decrease to about a 15% decrease, about a 5% decrease to about a 10% decrease, about a 10% decrease to about a 99% decrease, about a 10% decrease to about a 95% decrease, about a 10% decrease to about a 90% decrease, about a 10% decrease to about a 85% decrease, about a 10% decrease to about a 80% decrease, about a 10% decrease to about a 75% decrease, about a 10% decrease to about a 70% decrease, about a 10% decrease to about a 65% decrease, about a 10% decrease to about a 60% decrease, about a 10% decrease to about a 55% decrease, about a 10% decrease to about a 50% decrease, about a 10% decrease to about a 45% decrease, about a 10% decrease to about a 40% decrease, about a 10% decrease to about a 35% decrease, about a 10% decrease to about a 30% decrease, about a 10% decrease to about a 25% decrease, about a 10% decrease to about a 20% decrease, about a 10% decrease to about a 15% decrease, about a 15% decrease to about a 99% decrease, about a 15% decrease to about a 95% decrease, about a 15% decrease to about a 90% decrease, about a 15% decrease to about a 85% decrease, about a 15% decrease to about a 80% decrease, about a 15% decrease to about a 75% decrease, about a 15% decrease to about a 70% decrease, about a 15% decrease to about a 65% decrease, about a 15% decrease to about a 60% decrease, about a 15% decrease to about a 55% decrease, about a 15% decrease to about a 50% decrease, about a 15% decrease to about a 45% decrease, about a 15% decrease to about a 40% decrease, about a 15% decrease to about a 35% decrease, about a 15% decrease to about a 30% decrease, about a 15% decrease to about a 25% decrease, about a 15% decrease to about a 20% decrease, about a 20% decrease to about a 99% decrease, about a 20% decrease to about a 95% decrease, about a 20% decrease to about a 90% decrease, about a 20% decrease to about a 85% decrease, about a 20% decrease to about a 80% decrease, about a 20% decrease to about a 75% decrease, about a 20% decrease to about a 70% decrease, about a 20% decrease to about a 65% decrease, about a 20% decrease to about a 60% decrease, about a 20% decrease to about a 55% decrease, about a 20% decrease to about a 50% decrease, about a 20% decrease to about a 45% decrease, about a 20% decrease to about a 40% decrease, about a 20% decrease to about a 35% decrease, about a 20% decrease to about a 30% decrease, about a 20% decrease to about a 25% decrease, about a 25% decrease to about a 99% decrease, about a 25% decrease to about a 95% decrease, about a 25% decrease to about a 90% decrease, about a 25% decrease to about a 85% decrease, about a 25% decrease to about a 80% decrease, about a 25% decrease to about a 75% decrease, about a 25% decrease to about a 70% decrease, about a 25% decrease to about a 65% decrease, about a 25% decrease to about a 60% decrease, about a 25% decrease to about a 55% decrease, about a 25% decrease to about a 50% decrease, about a 25% decrease to about a 45% decrease, about a 25% decrease to about a 40% decrease, about a 25% decrease to about a 35% decrease, about a 25% decrease to about a 30% decrease, about a 30% decrease to about a 99% decrease, about a 30% decrease to about a 95% decrease, about a 30% decrease to about a 90% decrease, about a 30% decrease to about a 85% decrease, about a 30% decrease to about a 80% decrease, about a 30% decrease to about a 75% decrease, about a 30% decrease to about a 70% decrease, about a 30% decrease to about a 65% decrease, about a 30% decrease to about a 60% decrease, about a 30% decrease to about a 55% decrease, about a 30% decrease to about a 50% decrease, about a 30% decrease to about a 45% decrease, about a 30% decrease to about a 40% decrease, about a 30% decrease to about a 35% decrease, about a 35% decrease to about a 99% decrease, about a 35% decrease to about a 95% decrease, about a 35% decrease to about a 90% decrease, about a 35% decrease to about a 85% decrease, about a 35% decrease to about a 80% decrease, about a 35% decrease to about a 75% decrease, about a 35% decrease to about a 70% decrease, about a 35% decrease to about a 65% decrease, about a 35% decrease to about a 60% decrease, about a 35% decrease to about a 55% decrease, about a 35% decrease to about a 50% decrease, about a 35% decrease to about a 45% decrease, about a 35% decrease to about a 40% decrease, about a 40% decrease to about a 99% decrease, about a 40% decrease to about a 95% decrease, about a 40% decrease to about a 90% decrease, about a 40% decrease to about a 85% decrease, about a 40% decrease to about a 80% decrease, about a 40% decrease to about a 75% decrease, about a 40% decrease to about a 70% decrease, about a 40% decrease to about a 65% decrease, about a 40% decrease to about a 60% decrease, about a 40% decrease to about a 55% decrease, about a 40% decrease to about a 50% decrease, about a 40% decrease to about a 45% decrease, about a 45% decrease to about a 99% decrease, about a 45% decrease to about a 95% decrease, about a 45% decrease to about a 90% decrease, about a 45% decrease to about a 85% decrease, about a 45% decrease to about a 80% decrease, about a 45% decrease to about a 75% decrease, about a 45% decrease to about a 70% decrease, about a 45% decrease to about a 65% decrease, about a 45% decrease to about a 60% decrease, about a 45% decrease to about a 55% decrease, about a 45% decrease to about a 50% decrease, about a 50% decrease to about a 99% decrease, about a 50% decrease to about a 95% decrease, about a 50% decrease to about a 90% decrease, about a 50% decrease to about a 85% decrease, about a 50% decrease to about a 80% decrease, about a 50% decrease to about a 75% decrease, about a 50% decrease to about a 70% decrease, about a 50% decrease to about a 65% decrease, about a 50% decrease to about a 60% decrease, about a 50% decrease to about a 55% decrease, about a 55% decrease to about a 99% decrease, about a 55% decrease to about a 95% decrease, about a 55% decrease to about a 90% decrease, about a 55% decrease to about a 85% decrease, about a 55% decrease to about a 80% decrease, about a 55% decrease to about a 75% decrease, about a 55% decrease to about a 70% decrease, about a 55% decrease to about a 65% decrease, about a 55% decrease to about a 60% decrease, about a 60% decrease to about a 99% decrease, about a 60% decrease to about a 95% decrease, about a 60% decrease to about a 90% decrease, about a 60% decrease to about a 85% decrease, about a 60% decrease to about a 80% decrease, about a 60% decrease to about a 75% decrease, about a 60% decrease to about a 70% decrease, about a 60% decrease to about a 65% decrease, about a 65% decrease to about a 99% decrease, about a 65% decrease to about a 95% decrease, about a 65% decrease to about a 90% decrease, about a 65% decrease to about a 85% decrease, about a 65% decrease to about a 80% decrease, about a 65% decrease to about a 75% decrease, about a 65% decrease to about a 70% decrease, about a 70% decrease to about a 99% decrease, about a 70% decrease to about a 95% decrease, about a 70% decrease to about a 90% decrease, about a 70% decrease to about a 85% decrease, about a 70% decrease to about a 80% decrease, about a 70% decrease to about a 75% decrease, about a 75% decrease to about a 99% decrease, about a 75% decrease to about a 95% decrease, about a 75% decrease to about a 90% decrease, about a 75% decrease to about a 85% decrease, about a 75% decrease to about a 80% decrease, about a 80% decrease to about a 99% decrease, about a 80% decrease to about a 95% decrease, about a 80% decrease to about a 90% decrease, about a 80% decrease to about a 85% decrease, about a 85% decrease to about a 99% decrease, about a 85% decrease to about a 95% decrease, about a 85% decrease to about a 90% decrease, about a 90% decrease to about a 99% decrease, about a 90% decrease to about a 95% decrease, or about a 95% decrease to about a 99% decrease) in the size and/or volume of a tumor in the subject or population of subjects, e.g., as compared to the size and/or volume of the tumor prior to administration or compared to similar subjects not receiving a treatment or receiving a different treatment. In some embodiments, the size and/or volume of a tumor in a subject can be assessed by X-ray, ultrasound, computer tomography (CT) scan, magnetic resonance imaging (MRI), and positron-emission tomography (PET).

In some embodiments, the methods described herein can result in a decrease (e.g., at least a 1% decrease, at least a 5% decrease, at least a 10% decrease, at least a 15% decrease, at least a 20% decrease, at least a 25% decrease, at least a 30% decrease, at least a 35% decrease, at least a 40% decrease, at least a 45% decrease, at least a 50% decrease, at least a 55% decrease, at least a 60% decrease, at least a 65% decrease, at least a 70% decrease, at least a 75% decrease, at least a 80% decrease, at least a 85% decrease, at least a 90% decrease, at least a 95% decrease, or at least a 99% decrease, or about a 1% decrease to about a 99% decrease (or any of the subranges of this range described herein)), in the rate of growth of a tumor in the subject or population of subjects, e.g., as compared to the rate of growth of the tumor in the subject prior to administration or compared to similar subjects not receiving a treatment or receiving a different treatment. In some embodiments, the rate of growth of a tumor in a subject can be determined by imaging the subject over time, e.g., using X-ray, ultrasound, computer tomography (CT) scan, magnetic resonance imaging (MRI), and positron-emission tomography (PET).

Also provided herein are methods of improving the objective response rate in subjects (e.g., any of the exemplary subjects described herein) having unresectable advanced/metastatic pancreatic cancer that include administering to the subjects a therapeutically effective amount of a multi-chain chimeric polypeptide, wherein the multi-chain chimeric polypeptide comprises: (a) a first chimeric polypeptide comprising: (i) a first target-binding domain; (ii) a soluble tissue factor domain; and (iii) a first domain of a pair of affinity domains; and (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII. Any of the exemplary multi-chain chimeric polypeptides described herein can be used in these methods.

As used herein, the term “objective response rate” refers to international criteria proposed by the Response Evaluation Criteria in Solid Tumors Committee (RECIST) v1.1 (as described in Eisenhauer et al., Eur. J. Cancer 45:228-247, 2009).

In some embodiments, the methods can result in an increase (e.g., at least a 1% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase, at least a 95% increase, at least a 100% increase, at least a 110% increase, at least a 120% increase, at least a 130% increase, at least a 140% increase, at least a 150% increase, at least a 160% increase, at least a 170% increase, at least a 180% increase, at least a 190% increase, at least a 200% increase, at least a 210% increase, at least a 220% increase, at least a 230% increase, at least a 240% increase, at least a 250% increase, at least a 260% increase, at least a 270% increase, at least a 280% increase, at least a 290% increase, or at least a 300% increase, or about a 1% increase to about a 300% increase, about a 1% increase to about a 280% increase, about a 1% increase to about a 260% increase, about a 1% increase to about a 240% increase, about a 1% increase to about a 220% increase, about a 1% increase to about a 200% increase, about a 1% increase to about a 180% increase, about a 1% increase to about a 160% increase, about a 1% increase to about a 140% increase, about a 1% increase to about a 120% increase, about a 1% increase to about a 100% increase, about a 1% increase to about a 95% increase, about a 1% increase to about a 90% increase, about a 1% increase to about a 85% increase, about a 1% increase to about a 80% increase, about a 1% increase to about a 75% increase, about a 1% increase to about a 70% increase, about a 1% increase to about a 65% increase, about a 1% increase to about a 60% increase, about a 1% increase to about a 55% increase, about a 1% increase to about a 50% increase, about a 1% increase to about a 45% increase, about a 1% increase to about a 40% increase, about a 1% increase to about a 35% increase, about a 1% increase to about a 30% increase, about a 1% increase to about a 25% increase, about a 1% increase to about a 20% increase, about a 1% increase to about a 15% increase, about a 1% increase to about a 10% increase, about a 1% increase to about a 5% increase, about a 5% increase to about a 300% increase, about a 5% increase to about a 280% increase, about a 5% increase to about a 260% increase, about a 5% increase to about a 240% increase, about a 5% increase to about a 220% increase, about a 5% increase to about a 200% increase, about a 5% increase to about a 180% increase, about a 5% increase to about a 160% increase, about a 5% increase to about a 140% increase, about a 5% increase to about a 120% increase, about a 5% increase to about a 100% increase, about a 5% increase to about a 95% increase, about a 5% increase to about a 90% increase, about a 5% increase to about a 85% increase, about a 5% increase to about a 80% increase, about a 5% increase to about a 75% increase, about a 5% increase to about a 70% increase, about a 5% increase to about a 65% increase, about a 5% increase to about a 60% increase, about a 5% increase to about a 55% increase, about a 5% increase to about a 50% increase, about a 5% increase to about a 45% increase, about a 5% increase to about a 40% increase, about a 5% increase to about a 35% increase, about a 5% increase to about a 30% increase, about a 5% increase to about a 25% increase, about a 5% increase to about a 20% increase, about a 5% increase to about a 15% increase, about a 5% increase to about a 10% increase, about a 10% increase to about a 300% increase, about a 10% increase to about a 280% increase, about a 10% increase to about a 260% increase, about a 10% increase to about a 240% increase, about a 10% increase to about a 220% increase, about a 10% increase to about a 200% increase, about a 10% increase to about a 180% increase, about a 10% increase to about a 160% increase, about a 10% increase to about a 140% increase, about a 10% increase to about a 120% increase, about a 10% increase to about a 100% increase, about a 10% increase to about a 95% increase, about a 10% increase to about a 90% increase, about a 10% increase to about a 85% increase, about a 10% increase to about a 80% increase, about a 10% increase to about a 75% increase, about a 10% increase to about a 70% increase, about a 10% increase to about a 65% increase, about a 10% increase to about a 60% increase, about a 10% increase to about a 55% increase, about a 10% increase to about a 50% increase, about a 10% increase to about a 45% increase, about a 10% increase to about a 40% increase, about a 10% increase to about a 35% increase, about a 10% increase to about a 30% increase, about a 10% increase to about a 25% increase, about a 10% increase to about a 20% increase, about a 10% increase to about a 15% increase, about a 15% increase to about a 300% increase, about a 15% increase to about a 280% increase, about a 15% increase to about a 260% increase, about a 15% increase to about a 240% increase, about a 15% increase to about a 220% increase, about a 15% increase to about a 200% increase, about a 15% increase to about a 180% increase, about a 15% increase to about a 160% increase, about a 15% increase to about a 140% increase, about a 15% increase to about a 120% increase, about a 15% increase to about a 100% increase, about a 15% increase to about a 95% increase, about a 15% increase to about a 90% increase, about a 15% increase to about a 85% increase, about a 15% increase to about a 80% increase, about a 15% increase to about a 75% increase, about a 15% increase to about a 70% increase, about a 15% increase to about a 65% increase, about a 15% increase to about a 60% increase, about a 15% increase to about a 55% increase, about a 15% increase to about a 50% increase, about a 15% increase to about a 45% increase, about a 15% increase to about a 40% increase, about a 15% increase to about a 35% increase, about a 15% increase to about a 30% increase, about a 15% increase to about a 25% increase, about a 15% increase to about a 20% increase, about a 20% increase to about a 300% increase, about a 20% increase to about a 280% increase, about a 20% increase to about a 260% increase, about a 20% increase to about a 240% increase, about a 20% increase to about a 220% increase, about a 20% increase to about a 200% increase, about a 20% increase to about a 180% increase, about a 20% increase to about a 160% increase, about a 20% increase to about a 140% increase, about a 20% increase to about a 120% increase, about a 20% increase to about a 100% increase, about a 20% increase to about a 95% increase, about a 20% increase to about a 90% increase, about a 20% increase to about a 85% increase, about a 20% increase to about a 80% increase, about a 20% increase to about a 75% increase, about a 20% increase to about a 70% increase, about a 20% increase to about a 65% increase, about a 20% increase to about a 60% increase, about a 20% increase to about a 55% increase, about a 20% increase to about a 50% increase, about a 20% increase to about a 45% increase, about a 20% increase to about a 40% increase, about a 20% increase to about a 35% increase, about a 20% increase to about a 30% increase, about a 20% increase to about a 25% increase, about a 25% increase to about a 300% increase, about a 25% increase to about a 280% increase, about a 25% increase to about a 260% increase, about a 25% increase to about a 240% increase, about a 25% increase to about a 220% increase, about a 25% increase to about a 200% increase, about a 25% increase to about a 180% increase, about a 25% increase to about a 160% increase, about a 25% increase to about a 140% increase, about a 25% increase to about a 120% increase, about a 25% increase to about a 100% increase, about a 25% increase to about a 95% increase, about a 25% increase to about a 90% increase, about a 25% increase to about a 85% increase, about a 25% increase to about a 80% increase, about a 25% increase to about a 75% increase, about a 25% increase to about a 70% increase, about a 25% increase to about a 65% increase, about a 25% increase to about a 60% increase, about a 25% increase to about a 55% increase, about a 25% increase to about a 50% increase, about a 25% increase to about a 45% increase, about a 25% increase to about a 40% increase, about a 25% increase to about a 35% increase, about a 25% increase to about a 30% increase, about a 30% increase to about a 300% increase, about a 30% increase to about a 280% increase, about a 30% increase to about a 260% increase, about a 30% increase to about a 240% increase, about a 30% increase to about a 220% increase, about a 30% increase to about a 200% increase, about a 30% increase to about a 180% increase, about a 30% increase to about a 160% increase, about a 30% increase to about a 140% increase, about a 30% increase to about a 120% increase, about a 30% increase to about a 100% increase, about a 30% increase to about a 95% increase, about a 30% increase to about a 90% increase, about a 30% increase to about a 85% increase, about a 30% increase to about a 80% increase, about a 30% increase to about a 75% increase, about a 30% increase to about a 70% increase, about a 30% increase to about a 65% increase, about a 30% increase to about a 60% increase, about a 30% increase to about a 55% increase, about a 30% increase to about a 50% increase, about a 30% increase to about a 45% increase, about a 30% increase to about a 40% increase, about a 30% increase to about a 35% increase, about a 35% increase to about a 300% increase, about a 35% increase to about a 280% increase, about a 35% increase to about a 260% increase, about a 35% increase to about a 240% increase, about a 35% increase to about a 220% increase, about a 35% increase to about a 200% increase, about a 35% increase to about a 180% increase, about a 35% increase to about a 160% increase, about a 35% increase to about a 140% increase, about a 35% increase to about a 120% increase, about a 35% increase to about a 100% increase, about a 35% increase to about a 95% increase, about a 35% increase to about a 90% increase, about a 35% increase to about a 85% increase, about a 35% increase to about a 80% increase, about a 35% increase to about a 75% increase, about a 35% increase to about a 70% increase, about a 35% increase to about a 65% increase, about a 35% increase to about a 60% increase, about a 35% increase to about a 55% increase, about a 35% increase to about a 50% increase, about a 35% increase to about a 45% increase, about a 35% increase to about a 40% increase, about a 40% increase to about a 300% increase, about a 40% increase to about a 280% increase, about a 40% increase to about a 260% increase, about a 40% increase to about a 240% increase, about a 40% increase to about a 220% increase, about a 40% increase to about a 200% increase, about a 40% increase to about a 180% increase, about a 40% increase to about a 160% increase, about a 40% increase to about a 140% increase, about a 40% increase to about a 120% increase, about a 40% increase to about a 100% increase, about a 40% increase to about a 95% increase, about a 40% increase to about a 90% increase, about a 40% increase to about a 85% increase, about a 40% increase to about a 80% increase, about a 40% increase to about a 75% increase, about a 40% increase to about a 70% increase, about a 40% increase to about a 65% increase, about a 40% increase to about a 60% increase, about a 40% increase to about a 55% increase, about a 40% increase to about a 50% increase, about a 40% increase to about a 45% increase, about a 45% increase to about a 300% increase, about a 45% increase to about a 280% increase, about a 45% increase to about a 260% increase, about a 45% increase to about a 240% increase, about a 45% increase to about a 220% increase, about a 45% increase to about a 200% increase, about a 45% increase to about a 180% increase, about a 45% increase to about a 160% increase, about a 45% increase to about a 140% increase, about a 45% increase to about a 120% increase, about a 45% increase to about a 100% increase, about a 45% increase to about a 95% increase, about a 45% increase to about a 90% increase, about a 45% increase to about a 85% increase, about a 45% increase to about a 80% increase, about a 45% increase to about a 75% increase, about a 45% increase to about a 70% increase, about a 45% increase to about a 65% increase, about a 45% increase to about a 60% increase, about a 45% increase to about a 55% increase, about a 45% increase to about a 50% increase, about a 50% increase to about a 300% increase, about a 50% increase to about a 280% increase, about a 50% increase to about a 260% increase, about a 50% increase to about a 240% increase, about a 50% increase to about a 220% increase, about a 50% increase to about a 200% increase, about a 50% increase to about a 180% increase, about a 50% increase to about a 160% increase, about a 50% increase to about a 140% increase, about a 50% increase to about a 120% increase, about a 50% increase to about a 100% increase, about a 50% increase to about a 95% increase, about a 50% increase to about a 90% increase, about a 50% increase to about a 85% increase, about a 50% increase to about a 80% increase, about a 50% increase to about a 75% increase, about a 50% increase to about a 70% increase, about a 50% increase to about a 65% increase, about a 50% increase to about a 60% increase, about a 50% increase to about a 55% increase, about a 55% increase to about a 300% increase, about a 55% increase to about a 280% increase, about a 55% increase to about a 260% increase, about a 55% increase to about a 240% increase, about a 55% increase to about a 220% increase, about a 55% increase to about a 200% increase, about a 55% increase to about a 180% increase, about a 55% increase to about a 160% increase, about a 55% increase to about a 140% increase, about a 55% increase to about a 120% increase, about a 55% increase to about a 100% increase, about a 55% increase to about a 95% increase, about a 55% increase to about a 90% increase, about a 55% increase to about a 85% increase, about a 55% increase to about a 80% increase, about a 55% increase to about a 75% increase, about a 55% increase to about a 70% increase, about a 55% increase to about a 65% increase, about a 55% increase to about a 60% increase, about a 60% increase to about a 300% increase, about a 60% increase to about a 280% increase, about a 60% increase to about a 260% increase, about a 60% increase to about a 240% increase, about a 60% increase to about a 220% increase, about a 60% increase to about a 200% increase, about a 60% increase to about a 180% increase, about a 60% increase to about a 160% increase, about a 60% increase to about a 140% increase, about a 60% increase to about a 120% increase, about a 60% increase to about a 100% increase, about a 60% increase to about a 95% increase, about a 60% increase to about a 90% increase, about a 60% increase to about a 85% increase, about a 60% increase to about a 80% increase, about a 60% increase to about a 75% increase, about a 60% increase to about a 70% increase, about a 60% increase to about a 65% increase, about a 65% increase to about a 300% increase, about a 65% increase to about a 280% increase, about a 65% increase to about a 260% increase, about a 65% increase to about a 240% increase, about a 65% increase to about a 220% increase, about a 65% increase to about a 200% increase, about a 65% increase to about a 180% increase, about a 65% increase to about a 160% increase, about a 65% increase to about a 140% increase, about a 65% increase to about a 120% increase, about a 65% increase to about a 100% increase, about a 65% increase to about a 95% increase, about a 65% increase to about a 90% increase, about a 65% increase to about a 85% increase, about a 65% increase to about a 80% increase, about a 65% increase to about a 75% increase, about a 65% increase to about a 70% increase, about a 70% increase to about a 300% increase, about a 70% increase to about a 280% increase, about a 70% increase to about a 260% increase, about a 70% increase to about a 240% increase, about a 70% increase to about a 220% increase, about a 70% increase to about a 200% increase, about a 70% increase to about a 180% increase, about a 70% increase to about a 160% increase, about a 70% increase to about a 140% increase, about a 70% increase to about a 120% increase, about a 70% increase to about a 100% increase, about a 70% increase to about a 95% increase, about a 70% increase to about a 90% increase, about a 70% increase to about a 85% increase, about a 70% increase to about a 80% increase, about a 70% increase to about a 75% increase, about a 75% increase to about a 300% increase, about a 75% increase to about a 280% increase, about a 75% increase to about a 260% increase, about a 75% increase to about a 240% increase, about a 75% increase to about a 220% increase, about a 75% increase to about a 200% increase, about a 75% increase to about a 180% increase, about a 75% increase to about a 160% increase, about a 75% increase to about a 140% increase, about a 75% increase to about a 120% increase, about a 75% increase to about a 100% increase, about a 75% increase to about a 95% increase, about a 75% increase to about a 90% increase, about a 75% increase to about a 85% increase, about a 75% increase to about a 80% increase, about a 80% increase to about a 300% increase, about a 80% increase to about a 280% increase, about a 80% increase to about a 260% increase, about a 80% increase to about a 240% increase, about a 80% increase to about a 220% increase, about a 80% increase to about a 200% increase, about a 80% increase to about a 180% increase, about a 80% increase to about a 160% increase, about a 80% increase to about a 140% increase, about a 80% increase to about a 120% increase, about a 80% increase to about a 100% increase, about a 80% increase to about a 95% increase, about a 80% increase to about a 90% increase, about a 80% increase to about a 85% increase, about a 85% increase to about a 300% increase, about a 85% increase to about a 280% increase, about a 85% increase to about a 260% increase, about a 85% increase to about a 240% increase, about a 85% increase to about a 220% increase, about a 85% increase to about a 200% increase, about a 85% increase to about a 180% increase, about a 85% increase to about a 160% increase, about a 85% increase to about a 140% increase, about a 85% increase to about a 120% increase, about a 85% increase to about a 100% increase, about a 85% increase to about a 95% increase, about a 85% increase to about a 90% increase, about a 90% increase to about a 300% increase, about a 90% increase to about a 280% increase, about a 90% increase to about a 260% increase, about a 90% increase to about a 240% increase, about a 90% increase to about a 220% increase, about a 90% increase to about a 200% increase, about a 90% increase to about a 180% increase, about a 90% increase to about a 160% increase, about a 90% increase to about a 140% increase, about a 90% increase to about a 120% increase, about a 90% increase to about a 100% increase, about a 90% increase to about a 95% increase, about a 95% increase to about a 300% increase, about a 95% increase to about a 280% increase, about a 95% increase to about a 260% increase, about a 95% increase to about a 240% increase, about a 95% increase to about a 220% increase, about a 95% increase to about a 200% increase, about a 95% increase to about a 180% increase, about a 95% increase to about a 160% increase, about a 95% increase to about a 140% increase, about a 95% increase to about a 120% increase, about a 95% increase to about a 100% increase, about a 100% increase to about a 300% increase, about a 100% increase to about a 280% increase, about a 100% increase to about a 260% increase, about a 100% increase to about a 240% increase, about a 100% increase to about a 220% increase, about a 100% increase to about a 200% increase, about a 100% increase to about a 180% increase, about a 100% increase to about a 160% increase, about a 100% increase to about a 140% increase, about a 100% increase to about a 120% increase, about a 120% increase to about a 300% increase, about a 120% increase to about a 280% increase, about a 120% increase to about a 260% increase, about a 120% increase to about a 240% increase, about a 120% increase to about a 220% increase, about a 120% increase to about a 200% increase, about a 120% increase to about a 180% increase, about a 120% increase to about a 160% increase, about a 120% increase to about a 140% increase, about a 140% increase to about a 300% increase, about a 140% increase to about a 280% increase, about a 140% increase to about a 260% increase, about a 140% increase to about a 240% increase, about a 140% increase to about a 220% increase, about a 140% increase to about a 200% increase, about a 140% increase to about a 180% increase, about a 140% increase to about a 160% increase, about a 160% increase to about a 300% increase, about a 160% increase to about a 280% increase, about a 160% increase to about a 260% increase, about a 160% increase to about a 240% increase, about a 160% increase to about a 220% increase, about a 160% increase to about a 200% increase, about a 160% increase to about a 180% increase, about a 180% increase to about a 300% increase, about a 180% increase to about a 280% increase, about a 180% increase to about a 260% increase, about a 180% increase to about a 240% increase, about a 180% increase to about a 220% increase, about a 180% increase to about a 200% increase, about a 200% increase to about a 300% increase, about a 200% increase to about a 280% increase, about a 200% increase to about a 260% increase, about a 200% increase to about a 240% increase, about a 200% increase to about a 220% increase, about a 220% increase to about a 300% increase, about a 220% increase to about a 280% increase, about a 220% increase to about a 260% increase, about a 220% increase to about a 240% increase, about a 240% increase to about a 300% increase, about a 240% increase to about a 280% increase, about a 240% increase to about a 260% increase, about a 260% increase to about a 300% increase, about a 260% increase to about a 280% increase, or about a 280% increase to about a 300% increase) in the objective response rate in a subject or population of subjects, e.g., as compared to similar subjects not receiving a treatment or receiving a different treatment.

Also provided herein are methods of increasing progression-free survival or progression-free survival rate in a subject or population of subjects (e.g., any of the exemplary subjects described herein) having unresectable advanced/metastatic pancreatic cancer that include administering to the subject(s) a therapeutically effective amount of a multi-chain chimeric polypeptide, wherein the multi-chain chimeric polypeptide comprises: (a) a first chimeric polypeptide comprising: (i) a first target-binding domain; (ii) a soluble tissue factor domain; and (iii) a first domain of a pair of affinity domains; and (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII. Any of the exemplary multi-chain chimeric polypeptides described herein can be used in these methods.

As used herein, the term “progression-free survival” refers to a length of time during and/or after treatment that a subject survives without the cancer progressing. Progression-free survival can be based, e.g., on anatomical measurement of tumor size or volume, e.g., as determined using X-ray, ultrasound, computer tomography (CT) scan, magnetic resonance imaging (MRI), and positron-emission tomography (PET). The term “progression-free survival rate” refers to the percentage of subjects surviving without the cancer progression at a defined time since the initiation of treatment (e.g., 6 months, 1 year, etc.).

In some embodiments, the methods described herein provide for an increase (e.g., at least a 1% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase, at least a 95% increase, at least a 100% increase, at least a 110% increase, at least a 120% increase, at least a 130% increase, at least a 140% increase, at least a 150% increase, at least a 160% increase, at least a 170% increase, at least a 180% increase, at least a 190% increase, at least a 200% increase, at least a 210% increase, at least a 220% increase, at least a 230% increase, at least a 240% increase, at least a 250% increase, at least a 260% increase, at least a 270% increase, at least a 280% increase, at least a 290% increase, or at least a 300% increase, or about a 1% increase to about a 300% increase (or any of the subranges of this range described herein)) in progression-free survival or progression-free survival rate in the subject or population of subjects, e.g., as compared to the progression-free survival or progression-free survival rate in the subjects prior to the administering of the multi-chain chimeric polypeptide or as compared to the progression-free survival or progression-free survival rate in similar subjects administered a different treatment (e.g., any of the exemplary first-line and/or second-line treatments for pancreatic cancer described herein).

Also provided herein are methods of increasing time to progression in a subject or population of subjects (e.g., any of the exemplary subjects described herein) having unresectable advanced/metastatic pancreatic cancer that include administering to the subject(s) a therapeutically effective amount of a multi-chain chimeric polypeptide, wherein the multi-chain chimeric polypeptide comprises: (a) a first chimeric polypeptide comprising: (i) a first target-binding domain; (ii) a soluble tissue factor domain; and (iii) a first domain of a pair of affinity domains; and (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII. Any of the exemplary multi-chain chimeric polypeptides described herein can be used in these methods.

As used herein, the term “time to progression” can refer to a length of time from the start of treatment until the cancer progresses and/or metastasizes to other parts of the body in the subject. Cancer progression and/or metastasis can be determined, e.g., by imaging the subject, e.g., using X-ray, ultrasound, computer tomography (CT) scan, magnetic resonance imaging (MRI), and positron-emission tomography (PET).

In some embodiments, the methods described herein result in an increase (e.g., at least a 1% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase, at least a 95% increase, at least a 100% increase, at least a 110% increase, at least a 120% increase, at least a 130% increase, at least a 140% increase, at least a 150% increase, at least a 160% increase, at least a 170% increase, at least a 180% increase, at least a 190% increase, at least a 200% increase, at least a 210% increase, at least a 220% increase, at least a 230% increase, at least a 240% increase, at least a 250% increase, at least a 260% increase, at least a 270% increase, at least a 280% increase, at least a 290% increase, or at least a 300% increase, or about a 1% increase to about a 300% increase (or any of the subranges of this range described herein)) in the time to progression in the subject or population of subjects, e.g., as compared to the time to progression in the subjects prior to the administering of the multi-chain chimeric polypeptide or as compared to the time to progression in similar subjects administered a different treatment (e.g., any of the exemplary first-line and/or second-line treatments for pancreatic cancer described herein).

Also provided herein are methods of increasing duration of response in a subject or population of subjects (e.g., any of the exemplary subjects described herein) having unresectable advanced/metastatic pancreatic cancer that include administering to the subject(s) a therapeutically effective amount of a multi-chain chimeric polypeptide, wherein the multi-chain chimeric polypeptide comprises: (a) a first chimeric polypeptide comprising: (i) a first target-binding domain; (ii) a soluble tissue factor domain; and (iii) a first domain of a pair of affinity domains; and (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII. Any of the exemplary multi-chain chimeric polypeptides described herein can be used in these methods.

As used herein, the term “duration of response” can refer to the length of time from response to treatment until progression of cancer in the subject. For example, the duration of response can be a measure of the length of time that a tumor continues to respond to a treatment without the cancer growing or metastasizing. In some examples, the growth or metastasis of pancreatic cancer in a subject can be determined by imaging the subject, e.g., using X-ray, ultrasound, computer tomography (CT) scan, magnetic resonance imaging (MRI), and positron-emission tomography (PET).

In some embodiments, the methods described herein can result in an increase (e.g., at least a 1% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase, at least a 95% increase, at least a 100% increase, at least a 110% increase, at least a 120% increase, at least a 130% increase, at least a 140% increase, at least a 150% increase, at least a 160% increase, at least a 170% increase, at least a 180% increase, at least a 190% increase, at least a 200% increase, at least a 210% increase, at least a 220% increase, at least a 230% increase, at least a 240% increase, at least a 250% increase, at least a 260% increase, at least a 270% increase, at least a 280% increase, at least a 290% increase, or at least a 300% increase, or about a 1% increase to about a 300% increase (or any of the subranges of this range described herein)) in the duration of response in the subject or population of subjects, e.g., as compared to the duration of response in the subjects prior to the administering of the multi-chain chimeric polypeptide (e.g., in response to prior administration of a first and/or second line therapy for pancreatic cancer) or as compared to the duration of response in similar subjects administered a different treatment (e.g., any of the exemplary first-line and/or second-line treatments for pancreatic cancer described herein).

Also provided herein are methods of increasing overall survival in a population of subjects (e.g., any of the exemplary subjects described herein) having unresectable advanced/metastatic pancreatic cancer, the method comprising administering to each subject of the population a therapeutically effective amount of a multi-chain chimeric polypeptide, wherein the multi-chain chimeric polypeptide comprises: (a) a first chimeric polypeptide comprising: (i) a first target-binding domain; (ii) a soluble tissue factor domain; and (iii) a first domain of a pair of affinity domains; and (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-receptor II (TGF-βRII) and the second target-binding domain binds specifically to a ligand of TGF-βRII. Any of the exemplary multi-chain chimeric polypeptides described herein can be used in these methods.

As used herein, the term “overall survival” can refer to a length of time from either the date of diagnosis or the start of treatment that a population of subjects are still alive. Overall survival can measure how long a population of subjects, who undergo a certain cancer treatment regimen, live compared to another population of similar subjects who are in a control group (e.g., receiving a different treatment, e.g., a first line and/or second line treatment for pancreatic cancer, e.g., any of the exemplary first line and/or second line treatments for pancreatic cancer described herein).

In some embodiments, the methods described herein result in an increase (e.g., at least a 1% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase, at least a 95% increase, at least a 100% increase, at least a 110% increase, at least a 120% increase, at least a 130% increase, at least a 140% increase, at least a 150% increase, at least a 160% increase, at least a 170% increase, at least a 180% increase, at least a 190% increase, at least a 200% increase, at least a 210% increase, at least a 220% increase, at least a 230% increase, at least a 240% increase, at least a 250% increase, at least a 260% increase, at least a 270% increase, at least a 280% increase, at least a 290% increase, or at least a 300% increase, or about a 1% increase to about a 300% increase (or any of the subranges of this range described herein)) in the overall survival of the subjects, e.g., as compared to another population of similar subjects who are in a control group (e.g., receiving a different treatment, e.g., a first line and/or second line treatment for pancreatic cancer, e.g., any of the exemplary first line and/or second line treatments for pancreatic cancer described herein).

As used herein, the term “subject” can refer to an organism, typically a mammal (e.g., a human). In some embodiments, a subject is a patient.

In some embodiments, the subject(s) has/have an age of 18 years or more (e.g., 19 years or more, 20 years or more, 25 years or more, 30 years or more, 35 years or more, 40 years or more, 45 years or more, 50 years or more, 55 years or more, 60 years or more, 65 years or more, 70 years or more, 75 years or more, 80 years or more, 85 years or more, 90 years or more, 95 years or more, or 100 years or more).

In some embodiments, the subject(s) has/have received previous treatment with standard first-line systemic therapy for pancreatic cancer, and the subject's/subjects' pancreatic cancer had progressed on and/or was intolerant to the previous treatment. In some embodiments, the subject(s) has/have received previous treatment with standard first-line systemic therapy for pancreatic cancer, and the subject(s) was/were intolerant to the first-line systemic therapy.

In some embodiments the standard first-line systemic therapy comprises one or more of: FOLFIRINOX, modified FOLFINIROX, gemcitabine, albumin-bound paclitaxel, cisplatin, erlotinib, capecitabine, docetaxel, fluoropyrimidine, and oxaliplatin.

In some embodiments, the first-line systemic therapy comprises one of: (i) FOLFIRINOX; (ii) modified FOLFIRINOX; (iii) gemcitabine and albumin-bound paclitaxel; (iv) gemcitabine and erlotinib; (v) gemcitabine; (vi) gemcitabine and capecitabine; (vii) gemcitabine, docetaxel, and capecitabine; and (viii) fluoropyrimidine and oxaliplatin.

In some embodiments, the subject(s) has/have previously been identified as having a BRCA1, BRCA2, or PALB2 mutation, and the first-line systemic therapy comprises one of: (i) FOLFIRINOX; (ii) modified FOLFIRINOX; and (iii) gemcitabine and cisplatin.

In some embodiments, the subject(s) has/have received previous treatment with second- or later-line systemic therapy for pancreatic cancer, and the subject's/subjects' pancreatic cancer had progressed on and/or was intolerant to the previous treatment. In some embodiments, the second- or later-line systemic therapy comprises one or more of: a different first-line systemic therapy (e.g., any of the exemplary first-line systemic therapies described herein), 5-fluorouracil, leucovorin, liposomal irinotecan, irinotecan, FOLFIRINOX, modified FOLFIRINOX, oxaliplatin, FOLFOX, capecitabine, gemcitabine, albumin-bound paclitaxel, cisplatin, erlotinib, pembrolizumab, larotrectinib, and entrectinib. In some embodiments, the second- or later-line systemic therapy is a different first-line systemic therapy (e.g., any of the exemplary first-line systemic therapies described herein).

In some embodiments, the second- or later-line systemic therapy comprises one of: (i) 5-fluorouracil, leucovorin, and liposomal irinotecan; (ii) 5-fluorouracil, leucovorin, and irinotecan (FOLFIRI); (iii) FOLFIRINOX or modified FOLFIRINOX; (iv) oxaliplatin, 5-fluorouracil, and leucovorin (OFF); (v) FOLFOX; (vi) capecitabine and oxaliplatin; (vii) capecitabine; and (viii) continuous infusion 5-fluorouracil.

In some embodiments, the subject(s) was/were previously treated with fluoropyrimidine-based therapy and the second- or later-line systemic therapy comprises one of: (i) gemcitabine; (ii) gemcitabine and albumin-bound paclitaxel; and (iii) gemcitabine with erlotinib.

In some embodiments, the subject(s) was/were previously treated with fluoropyrimidine-based therapy and was/were previously identified as having a BRCA1, BRCA2, or PALB2 mutation, and the second- or later-line systemic therapy comprises gemcitabine and cisplatin. In some embodiments, the subject(s) was/were previously treated with fluoropyrimidine-based therapy and has/have not received prior treatment with irinotecan, and the second- or later-line systemic therapy comprises 5-fluorouracil, leucovorin, and liposomal irinotecan.

In some embodiments, the subject(s) was/were previously identified as having an MSI-H or dMMR tumor, and the second- or later-line systemic therapy comprises pembrolizumab. In some embodiments, the subject(s) was/were previously identified as having a NTRK gene fusion, and the second- or later-line systemic therapy comprises larotrectinib or entrectinib.

In some embodiments, the subject(s) has/have distant metastatic disease. In some embodiments, the subject(s) has/have adequate cardiac, pulmonary, liver, and kidney function. In some embodiments, the subject(s) has/have an Eastern Cooperative Oncology Group (ECOG) performance status of 0, 1, or 2.

In some embodiments, the subject(s) has/have a life expectancy, prior to the administering step, of at least 12 weeks (e.g., at least 14 weeks, at least 16 weeks, at least 18 weeks, at least 20 weeks, at least 22 weeks, or at least 24 weeks). In some embodiments, subject(s), prior to the administering step, has/have been determined to have measurable disease as assessed by imaging studies. In some embodiments, the subject(s) has/have received prior radiation therapy at least four weeks before the administering step. In some embodiments, any acute effects of any prior therapy in the subject(s) has/have reduced to baseline or a grade less than or equal to 1 NCI CTCAE v5.0, before the administering step.

In some embodiments, the subject(s) has/have: an absolute neutrophil count of greater than or equal to 1,500/microliter; a platelet count of greater than or equal to 100,000/microliter; a hemoglobin level of greater than or equal to 9 g/dL; a glomerular filtration rate (GFR) of greater than 40 mL/min or serum creatinine level of less than or equal to 1.5×Upper Limit of Normal (ULN); a total bilirubin level of less than or equal to 2.0×ULN or less than or equal to 3.0×ULN for subjects having Gilbert's syndrome; and aspartate aminotransferase (AST), alanine aminotransferase (ALT), and alkaline phosphatase (ALP) levels of less than or equal to 2.5×ULN or less than or equal to 5.0×ULN if liver metastasis is present. In some embodiments, the subject(s) has/have a level of Pulmonary Function Test (PFT) greater than 50% Forced Expiratory Volume (FEV1) if symptomatic or prior known impairment.

In some embodiments, the subject(s) is/are female, and the female(s) has/have had a negative pregnancy test within 14 days prior to the administering step. In some embodiments, the female(s) has/have received birth control at least 14 days prior, and during, the administering step, or is surgically sterilized.

In some embodiments, the subject(s) is/are male, and the subject(s) uses/use barrier method birth control during the administering step, and at least 28 days after the administering step.

In some embodiments, the subject(s) does/do not have a history of clinically significant vascular disease. In some embodiments, the subject(s) does/do not have a Corrected QT interval (QTc) of greater than or equal to 470 milliseconds by Fridericia's correction.

In some embodiments, the subject(s) does/do not have an untreated CNS metastasis. In some embodiments, the subject(s) has/have received prior treatment for CNS metastasis and the subject(s) is/are neurologically stable for at least two weeks prior to the administering step. In some embodiments, the subject(s) is/are not receiving, during the administering step, a corticosteroid. In some embodiments, the subject(s) is/are receiving a stable or decreasing dose of a corticosteroid of less than or equal to 10 mg daily, during the administering step.

In some embodiments, the subject(s) has/have not received surgery, radiotherapy, chemotherapy, other immunotherapy, or investigational therapy within 14 days prior to the administering step. In some embodiments, the subject(s) does/do not have any other prior malignancy except for adequately-treated basal cell or squamous cell skin cancer, in situ cervical cancer, adequately-treated stage I or II cancer from which the subject(s) is/are currently in complete remission, or any other cancer from which the subject(s) has/have been disease-free for 3 years after surgical treatment.

In some embodiments, the subject(s) does/do not have known hypersensitivity or a history of allergic reactions attributed to compounds of similar chemical or biological composition to the multi-chain chimeric polypeptide. In some embodiments, the subject(s) has/have not received prior treatment with a TGF-beta antagonist or IL-15 or analog thereof.

In some embodiments, the subject(s) is/are not receiving concurrent herbal or unconventional therapy. In some embodiments, the subject(s) does/do not have an autoimmune disease requiring active treatment. In some embodiments, the subject(s) does/do not have a condition requiring systemic treatment with a corticosteroid or an immunosuppressive treatment within 14 days of the administering step. In some embodiments, the subject(s) does/do not have active autoimmune disease, and has received inhaled or topical steroids or adrenal replacement steroid doses of equal to or less than 10 mg daily prednisone equivalent.

In some embodiments, the subject(s) does/do not have an active systemic infection requiring parenteral antibiotic therapy. In some embodiments, the subject(s) has/have not previously received an organ allograft or allogeneic transplantation. In some embodiments, the subject(s) has/have not been identified or diagnosed as being HIV-positive or having AIDS.

In some embodiments, the subject(s) is/are a female and the female(s) is/are not pregnant or nursing. In some embodiments, the subject(s) does/do not have any ongoing toxicity from a prior treatment. In some embodiments, the ongoing toxicity is greater than grade 1 using NCI CTCAE v5.0 or greater than baseline. In some embodiments, the ongoing toxicity excludes peripheral neuropathy, alopecia, and fatigue.

In some embodiments, the subject(s) does/do not have psychiatric illness.

In some embodiments, the multi-chain chimeric polypeptide is subcutaneously administered to the subject(s). In some embodiments, the subject(s) is/are administered a single dose of the multi-chain chimeric polypeptide. In some embodiments, the single dose is about 0.1 mg of the multi-chain chimeric polypeptide per kg of the subject's body weight (mg/kg), about 0.25 mg/kg, about 0.5 mg/kg, about 0.8 mg/kg, or about 1.2 mg/kg.

In some embodiments, the subject(s) is/are administered two or more doses of the multi-chain chimeric polypeptide over a treatment period. In some embodiments, at least one of the two or more doses is 0.1 mg of the multi-chain chimeric polypeptide per kg of the subject's body weight (mg/kg), about 0.25 mg/kg, about 0.5 mg/kg, about 0.8 mg/kg, or about 1.2 mg/kg.

In some embodiments, the treatment period is about 4 weeks.

### EXAMPLES

The invention is further described in the following examples, which do not limit the scope of the invention described in the claims.

### Example 1: TGFRt15-TGFRs Fusion Protein Generation and Characterization

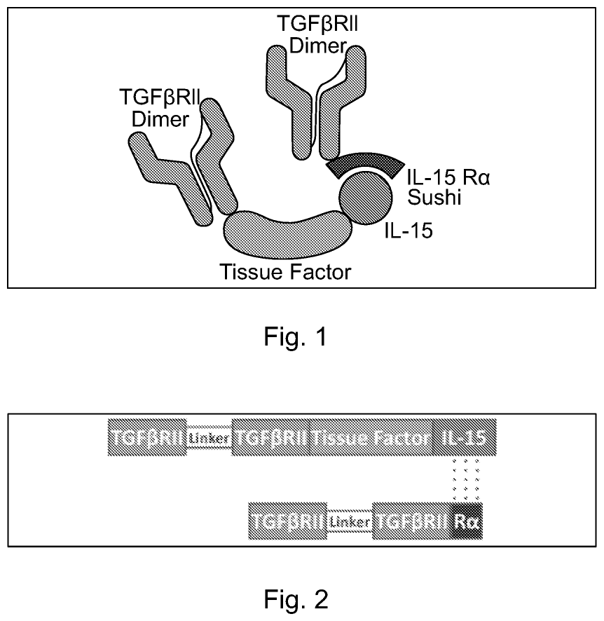

A fusion protein complex was generated comprising of TGFβ Receptor II/IL-15RαSu and TGF Receptor II/TF/IL-15 fusion proteins (FIG. 1 and FIG. 2). The human TGFβ Receptor II (Ile²⁴-Asp¹⁵⁹), tissue factor 219, and IL-15 sequences were obtained from the UniProt website and DNA for these sequences was synthesized by Genewiz. Specifically, a construct was made linking two TGFβ Receptor II sequences with a G4S(3) linker to generate a single chain version of TGFβ Receptor II and then directly linking to the N-terminus coding region of tissue factor 219 followed by the N-terminus coding region of IL-15.

The nucleic acid and protein sequences of a construct comprising two TGFβ Receptor II linked to the N-terminus of tissue factor 219 following with the N-terminus of IL-15 are shown below.

The nucleic acid sequence of the two TGFβ Receptor II/TF/IL-15 construct (including signal peptide sequence) is as follows:

(Signal peptide)
ATGAAGTGGGTGACCTTCATCAGCCTGCTGTTCCTGTTCTCCAGCGCCT
ACTCC
(Two Human TGFβ Receptor II fragments)
ATCCCCCCCCATGTGCAAAAGAGCGTGAACAACGATATGATCGTGACC
GACAACAACGGCGCCGTGAAGTTTCCCCAGCTCTGCAAGTTCTGCGATG
TCAGGTTCAGCACCTGCGATAATCAGAAGTCCTGCATGTCCAACTGCAG
CATCACCTCCATCTGCGAGAAGCCCCAAGAAGTGTGCGTGGCCGTGTGG
CGGAAAAATGACGAGAACATCACCCTGGAGACCGTGTGTCACGACCCCA
AGCTCCCTTATCACGACTTCATTCTGGAGGACGCTGCCTCCCCCAAATG
CATCATGAAGGAGAAGAAGAAGCCCGGAGAGACCTTCTTTATGTGTTCC
TGTAGCAGCGACGAGTGTAACGACAACATCATCTTCAGCGAAGAGTACA
ACACCAGCAACCCTGATGGAGGTGGCGGATCCGGAGGTGGAGGTTCTGG
TGGAGGTGGGAGTATTCCTCCCCACGTGCAGAAGAGCGTGAATAATGAC
ATGATCGTGACCGATAACAATGGCGCCGTGAAATTTCCCCAGCTGTGCA
AATTCTGCGATGTGAGGTTTTCCACCTGCGACAACCAGAAGTCCTGTAT
GAGCAACTGCTCCATCACCTCCATCTGTGAGAAGCCTCAGGAGGTGTGC
GTGGCTGTCTGGCGGAAGAATGACGAGAATATCACCCTGGAAACCGTCT
GCCACGATCCCAAGCTGCCCTACCACGATTTCATCCTGGAAGACGCCGC
CAGCCCTAAGTGCATCATGAAAGAGAAAAAGAAGCCTGGCGAGACCTTT
TTCATGTGCTCCTGCAGCAGCGACGAATGCAACGACAATATCATCTTTA
GCGAGGAATACAATACCAGCAACCCCGAC
(Human Tissue Factor 219)
AGCGGCACAACCAACACAGTCGCTGCCTATAACCTCACTTGGAAGAG
CACCAACTTCAAAACCATCCTCGAATGGGAACCCAAACCCGTTAACCAA
GTTTACACCGTGCAGATCAGCACCAAGTCCGGCGACTGGAAGTCCAAAT
GTTTCTATACCACCGACACCGAGTGCGATCTCACCGATGAGATCGTGAA
AGATGTGAAACAGACCTACCTCGCCCGGGTGTTTAGCTACCCCGCCGGC
AATGTGGAGAGCACTGGTTCCGCTGGCGAGCCTTTATACGAGAACAGCC
CCGAATTTACCCCTTACCTCGAGACCAATTTAGGACAGCCCACCATCCA
AAGCTTTGAGCAAGTTGGCACAAAGGTGAATGTGACAGTGGAGGACGAG
CGGACTTTAGTGCGGCGGAACAACACCTTTCTCAGCCTCCGGGATGTGT
TCGGCAAAGATTTAATCTACACACTGTATTACTGGAAGTCCTCTTCCTC
CGGCAAGAAGACAGCTAAAACCAACACAAACGAGTTTTTAATCGACGTG
GATAAAGGCGAAAACTACTGTTTCAGCGTGCAAGCTGTGATCCCCTCCC
GGACCGTGAATAGGAAAAGCACCGATAGCCCCGTTGAGTGCATGGGCCA
AGAAAAGGGCGAGTTCCGGGAG
(Human IL-15)
AACTGGGTGAACGTCATCAGCGATTTAAAGAAGATCGAAGATTTAATT
CAGTCCATGCATATCGACGCCACTTTATACACAGAATCCGACGTGCACC
CCTCTTGTAAGGTGACCGCCATGAAATGTTTTTTACTGGAGCTGCAAGT
TATCTCTTTAGAGAGCGGAGACGCTAGCATCCACGACACCGTGGAGAAT
TTAATCATTTTAGCCAATAACTCTTTATCCAGCAACGGCAACGTGACAG
AGTCCGGCTGCAAGGAGTGCGAAGAGCTGGAGGAGAAGAACATCAAGGA
GTTTCTGCAATCCTTTGTGCACATTGTCCAGATGTTCATCAATACCTCC

The amino acid sequence of TGFβ Receptor II/TF/IL-15 fusion protein (including the leader sequence) is as follows:

(Signal peptide)
MKWVTFISLLFLFSSAYS
(Human TGFβ Receptor II)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPD
(Human Tissue Factor 219)
SGTTNTVAAYNLTWKSTNFKTILEWEPKPVNQVYTVQISTKSGDWKSKC
FYTTDTECDLTDEIVKDVKQTYLARVFSYPAGNVESTGSAGEPLYENSP
EFTPYLETNLGQPTIQSFEQVGTKVNVTVEDERTLVRRNNTFLSLRDVF
GKDLIYTLYYWKSSSSGKKTAKTNTNEFLIDVDKGENYCFSVQAVIPSR
TVNRKSTDSPVECMGQEKGEFRE
(Human IL-15)
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS

Constructs were also made by attaching two TGFβ Receptor II directly to the IL-15RαSu chain which was synthesized by Genewiz. The nucleic acid and protein sequences of a construct comprising the TGF Receptor II linked to the N-terminus of IL-15RαSu are shown below.

The nucleic acid sequence of the TGFβ Receptor II/IL-15 RαSu construct (including signal peptide sequence) is as follows:

(Signal peptide)
ATGAAGTGGGTGACCTTCATCAGCCTGCTGTTCCTGTTCTCCAGCGCCT
ACTCC
(Two human TGFβ Receptor II fragments)
ATCCCCCCCCATGTGCAAAAGAGCGTGAACAACGATATGATCGTGACC
GACAACAACGGCGCCGTGAAGTTTCCCCAGCTCTGCAAGTTCTGCGATG
TCAGGTTCAGCACCTGCGATAATCAGAAGTCCTGCATGTCCAACTGCAG
CATCACCTCCATCTGCGAGAAGCCCCAAGAAGTGTGCGTGGCCGTGTGG
CGGAAAAATGACGAGAACATCACCCTGGAGACCGTGTGTCACGACCCCA
AGCTCCCTTATCACGACTTCATTCTGGAGGACGCTGCCTCCCCCAAATG
CATCATGAAGGAGAAGAAGAAGCCCGGAGAGACCTTCTTTATGTGTTCC
TGTAGCAGCGACGAGTGTAACGACAACATCATCTTCAGCGAAGAGTACA
ACACCAGCAACCCTGATGGAGGTGGCGGATCCGGAGGTGGAGGTTCTGG
TGGAGGTGGGAGTATTCCTCCCCACGTGCAGAAGAGCGTGAATAATGAC
ATGATCGTGACCGATAACAATGGCGCCGTGAAATTTCCCCAGCTGTGCA
AATTCTGCGATGTGAGGTTTTCCACCTGCGACAACCAGAAGTCCTGTAT
GAGCAACTGCTCCATCACCTCCATCTGTGAGAAGCCTCAGGAGGTGTGC
GTGGCTGTCTGGCGGAAGAATGACGAGAATATCACCCTGGAAACCGTCT
GCCACGATCCCAAGCTGCCCTACCACGATTTCATCCTGGAAGACGCCGC
CAGCCCTAAGTGCATCATGAAAGAGAAAAAGAAGCCTGGCGAGACCTTT
TTCATGTGCTCCTGCAGCAGCGACGAATGCAACGACAATATCATCTTTA
GCGAGGAATACAATACCAGCAACCCCGAC
(Human IL-15Rα sushi domain)
ATTACATGCCCCCCTCCCATGAGCGTGGAGCACGCCGACATCTGGGTG
AAGAGCTATAGCCTCTACAGCCGGGAGAGGTATATCTGTAACAGCGGCT
TCAAGAGGAAGGCCGGCACCAGCAGCCTCACCGAGTGCGTGCTGAATAA
GGCTACCAACGTGGCTCACTGGACAACACCCTCTTTAAAGTGCATCCGG

The amino acid sequence of the two TGFβ Receptor II/IL-15RαSu construct (including signal peptide sequence) is as follows:

(Signal peptide)
MKWVTFISLLFLFSSAYS
(Two human TGFβ Receptor II extra-cellular
domains)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPD
(Human IL-15Rα sushi domain)
ITCPPPMSVEHADIWVKSYSLYSRERYICNSGFKRKAGTSSLTECVLNK
ATNVAHWTTPSLKCIR

In some cases, the leader peptide is cleaved from the intact polypeptide to generate the mature form that may be soluble or secreted.

The TGFβR/IL-15RαSu and TGFβR/TF/IL-15 constructs were cloned into a modified retrovirus expression vectors as described previously (Hughes M S, Yu Y Y, Dudley M E, Zheng Z, Robbins P F, Li Y, et al. Transfer of a TCR gene derived from a patient with a marked antitumor response conveys highly active T-cell effector functions. Hum Gene Ther 2005; 16:457-72), and the expression vectors were transfected into CHO-K1 cells. Co-expression of the two constructs in CHO-K1 cells allowed for formation and secretion of the soluble TGFβR/TF/IL-15:TGFβR/IL-15RαSu protein complex (referred to as TGFRt15-TGFRs), which can be purified by anti-TF IgG1 affinity and other chromatography methods.

### Effect of TGFRt15-TGFRs on TGFβ1 Activity in HEK-Blue TGFβ Cells

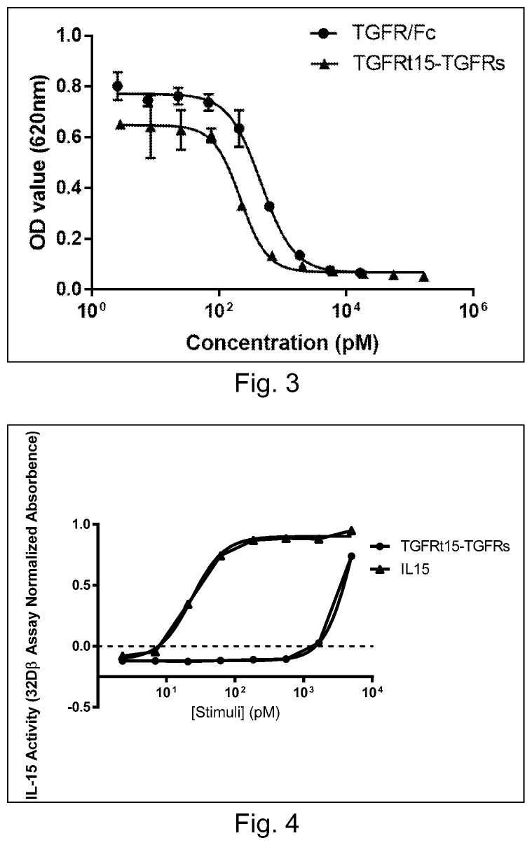

To evaluate the activity of TGFβRII in TGFRt15-TGFRs, the effect of TGFRt15-16s21 on the activity of TGFβ1 in HEK-Blue TGFβ cells was analyzed. HEK-Blue TGFβ cells (Invivogen) were washed twice with pre-warmed PBS and resuspended in the testing medium (DMEM, 10% heat-inactivated FCS, 1× glutamine, 1× anti-anti, and 2× glutamine) at 5×10⁵ cells/mL. In a flat-bottom 96-well plate, 50 μL cells were added to each well (2.5×10⁴ cells/well) and followed with 50 μL 0.1 nM TGFβ1 (R&D systems). TGFRt15-16s21 or TGFR-Fc (R&D Systems) prepared at a 1:3 serial dilution was then added to the plate to reach a total volume of 200 μL. After 24 hrs of incubation at 37° C., 40 μL of induced HEK-Blue TGFβ cell supernatant was added to 160 μL pre-warmed QUANTI-Blue (Invivogen) in a flat-bottom 96-well plate, and incubated at 37° C. for 1-3 hrs. The OD values were then determined using a plate reader (Multiscan Sky) at 620-655 nM (FIG. 3). The IC₅₀ of each protein sample was calculated with GraphPad Prism 7.04. The IC₅₀ of TGFRt15-TGFRs and TGFR-Fc were 216.9 pM and 460.6 pM respectively. These results showed that the TGFβRII domain in TGFRt15-TGFRs was able to block the activity of TGFβ1 in HEK-Blue TGFβ cells.

### The IL-15 in TGFRt15-TGFRs Promotes IL-2Rβ and Common γ Chain Containing 32Dβ Cell Proliferation

To evaluate the activity of IL-15 in TGFRt15-TGFRs, the IL-15 activity of TGFRt15-TGFRs was compared to recombinant IL-15 using 32Dβ cells that express IL2Rβ and common γ chain, and evaluating their effects on promoting cell proliferation. IL-15 dependent 32Dβ cells were washed 5 times with IMDM-10% FBS and seeded in the wells at 2×10⁴ cells/well. Serially-diluted TGFRt15-TGFRs or IL-15 were added to the cells (FIG. 4). Cells were incubated in a CO₂ incubator at 37° C. for 3 days. Cell proliferation was detected by adding 10 μL of WST1 to each well on day 3 and incubating for an additional 3 hours in a CO₂ incubator at 37° C. The absorbance at 450 nm was measured by analyzing the amount of formazan dye produced. As shown in FIG. 4, TGFRt1S-TGFRs and IL-15 promoted 32Dβ cell proliferation, with the EC₅₀ of TGFRt15-16s21 and IL-15 being 1901 pM and 10.63 pM, respectively.

Detection of IL-15 and TGFβRII Domains in TGFRt15-TGFRs with Corresponding Antibodies Using ELISA

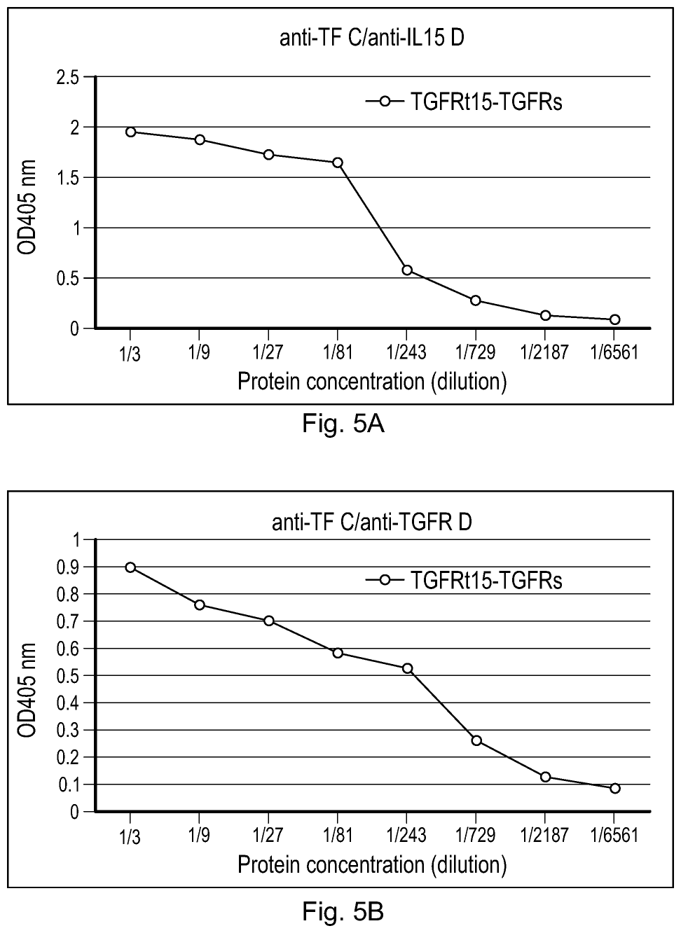

A 96-well plate was coated with 100 μL (8 μg/mL) of anti-TF IgG1 in R5 (coating buffer) and incubated at room temperature (RT) for 2 hrs. The plates were washed 3 times and blocked with 100 μL of 1% BSA in PBS. TGFRt15-TGFRs was added at a 1:3 serial dilution, and incubated at RT for 60 min. After 3 washes, 50 ng/mL of biotinylated-anti-IL-15 antibody (BAM247, R&D Systems), or 200 ng/ml of biotinylated-anti-TGFbRII antibody (BAF241, R&D Systems) was added to the wells and incubated at RT for 60 min. Next the plates were washed 3 times, and 0.25 μg/mL of HRP-SA (Jackson ImmunoResearch) at 100 μL per well was added and incubated for 30 min at RT, followed by 4 washes and incubation with 100 μL of ABTS for 2 mins at RT. Absorbance at 405 nm was read. As shown in FIGS. 5A and 5B, the IL-15 and TGFβRII domains in TGFRt15-TGFRs were detected by the individual antibodies.

Purification Elution Chromatograph of TGFRt15-TGFRs from Anti-TF Antibody Affinity Column

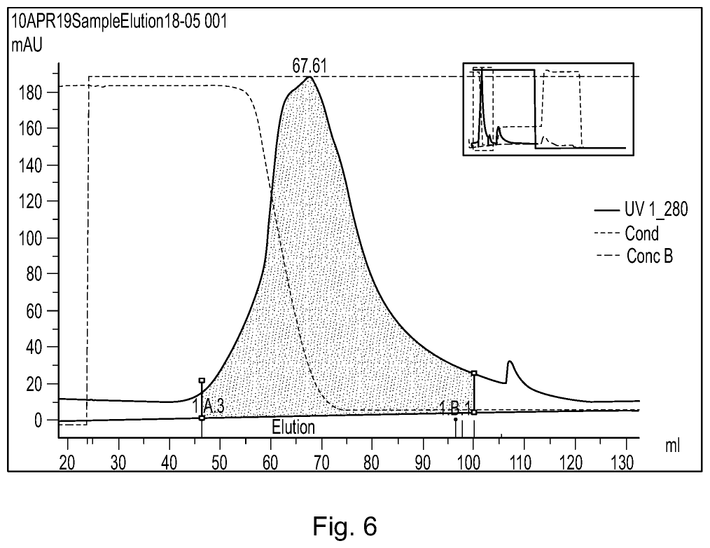

TGFRt15-TGFRs harvested from cell culture was loaded onto the anti-TF antibody affinity column equilibrated with 5 column volumes of PBS. After sample loading, the column was washed with 5 column volumes of PBS, followed by elution with 6 column volumes of 0.1M acetic acid (pH 2.9). A280 elution peak was collected and then neutralized to pH 7.5-8.0 with 1M Tris base. The neutralized sample was then buffer exchanged into PBS using Amicon centrifugal filters with a 30 KDa molecular weight cutoff. As shown in FIG. 6, the anti-TF antibody affinity column bound to TGFRt15-TGFRs which contains TF as a fusion partner. The buffer-exchanged protein sample was stored at 2-8° C. for further biochemical analyses and biological activity tests. After each elution, the anti-TF antibody affinity column was stripped using 6 column volumes of 0.1M glycine (pH 2.5). The column was then neutralized using 5 column volumes of PBS, and 7 column volumes of 20% ethanol for storage. The anti-TF antibody affinity column was connected to a GE Healthcare AKTA Avant system. The flow rate was 4 mL/min for all steps except for the elution step, which was 2 mL/min.

### Analytical Size Exclusion Chromatography (SEC) Analysis of TGFRt15-TGFRs

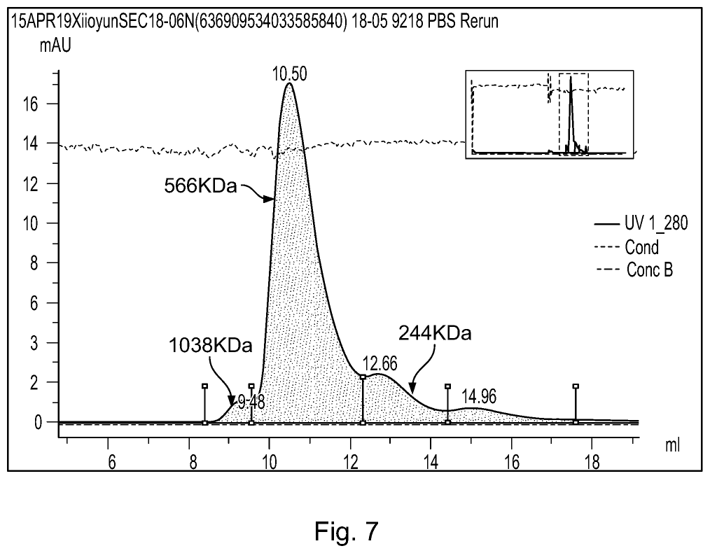

A Superdex 200 Increase 10/300 GL gel filtration column (from GE Healthcare) was connected to an AKTA Avant system (from GE Healthcare). The column was equilibrated with 2 column volumes of PBS. The flow rate was 0.7 mL/min. A sample containing TGFRt15-TGFRs in PBS was injected into the Superdex 200 column using a capillary loop, and analyzed by SEC. The SEC chromatograph of the sample is shown in FIG. 7. The SEC results showed four protein peaks for TGFRt15-TGFRs.

### Reduced SDS-PAGE Analysis of TGFRt15-TGFRs

To determine the purity and molecular weight of the TGFRt15-TGFRs protein, protein sample purified with anti-TF antibody affinity column was analyzed by sodium dodecyl sulfate polyacrylamide gel (4-12% NuPage Bis-Tris gel) electrophoresis (SDS-PAGE) method under reduced condition. After electrophoresis, the gel was stained with InstantBlue for about 30 min, followed by destaining overnight in purified water.

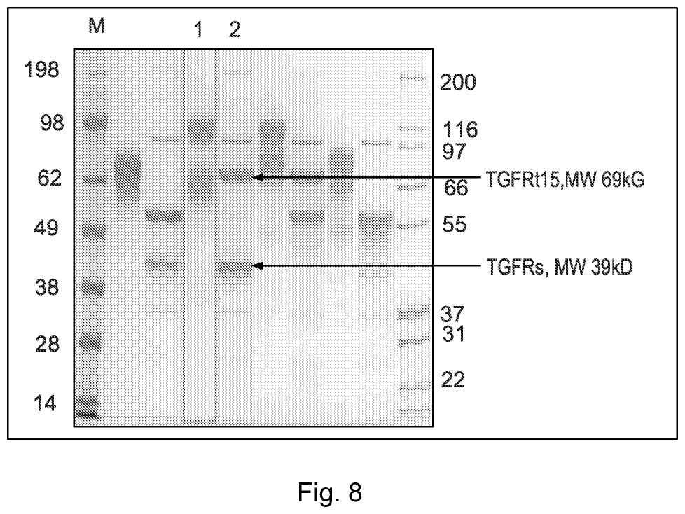

To verify that the TGFRt15-TGFRs protein undergoes glycosylation after translation in CHO cells, a deglycosylation experiment was conducted using the Protein Deglycosylation Mix II kit from New England Biolabs and the manufacturer's instructions. FIG. 8 shows the reduced SDS-PAGE analysis of the sample in non-deglycosylated (lane 1 in red outline) and deglycosylated (lane 2 in yellow outline) state. The results showed that the TGFRt15-TGFRs protein is glycosylated when expressed in CHO cells. After deglycosylation, the purified sample showed expected molecular weights (69 kDa and 39 kDa) in the reduced SDS gel. Lane M was loaded with 10 μl of SeeBlue Plus2 Prestained Standard.

### Immunostimulatory Activity of TGFRt15-TGFRs in C57BL/6 Mice

TGFRt15-TGFRs is a multi-chain polypeptide (a type A multi-chain polypeptide described herein) that includes a first polypeptide that is a soluble fusion of two TGFβRII domains, human tissue factor 219 fragment and human IL-15, and the second polypeptide that is a soluble fusion of two TGFβRII domains and sushi domain of human IL-15 receptor alpha chain.

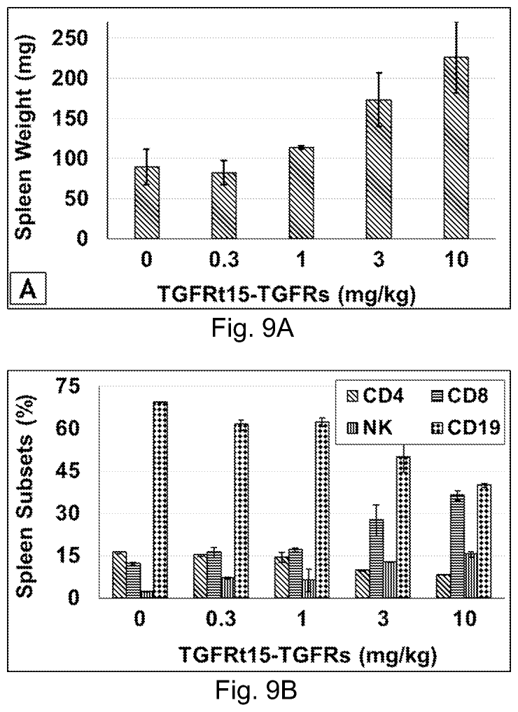

Wild type C57BL/6 mice were treated subcutaneously with either control solution or with TGFRt15-TGFRs at a dosage of 0.3 mg/kg, 1 mg/kg, 3 mg/kg, or 10 mg/kg. Four days after treatment, spleen weight and the percentages of various immune cell types present in the spleen were evaluated. As shown in FIG. 9A, the spleen weight in mice treated with TGFRt15-TGFRs increased with increasing dosage of TGFRt15-TGFRs. Moreover, the spleen weight in mice treated with 1 mg/kg, 3 mg/kg, and 10 mg/kg of TGFRt15-TGFRs were higher as compared to mice treated with the control solution, respectively. In addition, the percentages of CD4⁺ T cells, CD8⁺ T cells, NK cells, and CD19⁺ B cells present in the spleen of control-treated and TGFRt15-TGFRs-treated mice were evaluated. As shown in FIG. 9B, in the spleens of mice treated with TGFRt15-TGFRs, the percentages of CD8⁺ T cells and NK cells both increased with increasing dosage of TGFRt15-TGFRs. Specifically, the percentages of CD8⁺ T cells were higher in mice treated with 0.3 mg/kg, 3 mg/kg, and 10 mg/kg of TGFRt15-TGFRs compared to control-treated mice, and the percentages of NK cells were higher in mice treated with 0.3 mg/kg, 1 mg/kg, 3 mg/kg, and 10 mg/kg of TGFRt15-TGFRs compared to control-treated mice. These results demonstrate that TGFRt15-TGFRs is able to stimulate immune cells in the spleen, in particular CD8⁺ T cells and NK cells.

The pharmacokinetics of TGFRt15-TGFRs molecules were evaluated in wild type C57BL/6 mice. The mice were treated subcutaneously with TGFRt15-TGFRs at a dosage of 3 mg/kg. The mouse blood was drained from tail vein at various time points and the serum was prepared. The TGFRt15-TGFRs concentrations in mouse serum was determined with ELISA (capture: anti-human tissue factor antibody; detection: biotinylated anti-human TGFβ receptor antibody and followed by peroxidase conjugated streptavidin and ABTS substrate). The results showed that the half-life of TGFRt15-TGFRs was 12.66 hours in C57BL/6 mice.

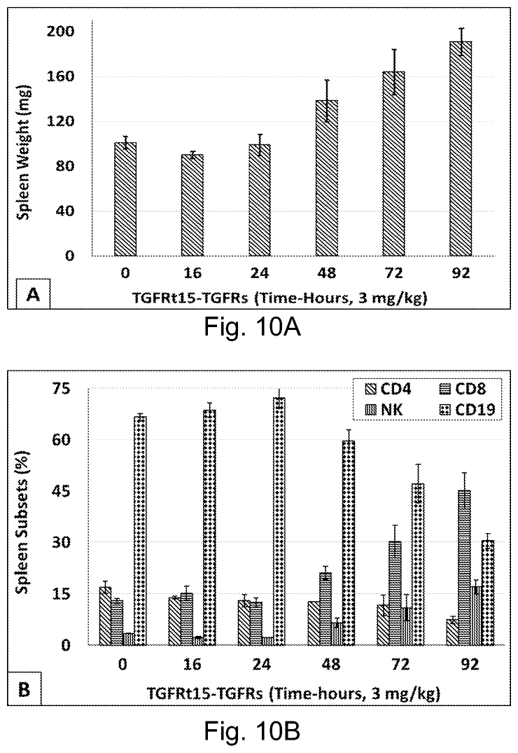

The mouse splenocytes were prepared in order to evaluate the immunostimulatory activity of TGFRt15-TGFRs over time in mice. As shown in FIG. 10A, the spleen weight in mice treated with TGFRt15-TGFRs increased 48 hours posttreatment and continued to increase over time. In addition, the percentages of CD4⁺ T cells, CD8⁺ T cells, NK cells, and CD19⁺ B cells present in the spleen of control-treated and TGFRt15-TGFRs-treated mice were evaluated. As shown in FIG. 10B, in the spleens of mice treated with TGFRt15-TGFRs, the percentages of CD8⁺ T cells and NK cells both increased at 48 hours after treatment and were higher and higher overtime after the single dose treatment. These results further demonstrate that TGFRt15-TGFRs is able to stimulate immune cells in the spleen, in particular CD8⁺ T cells and NK cells.

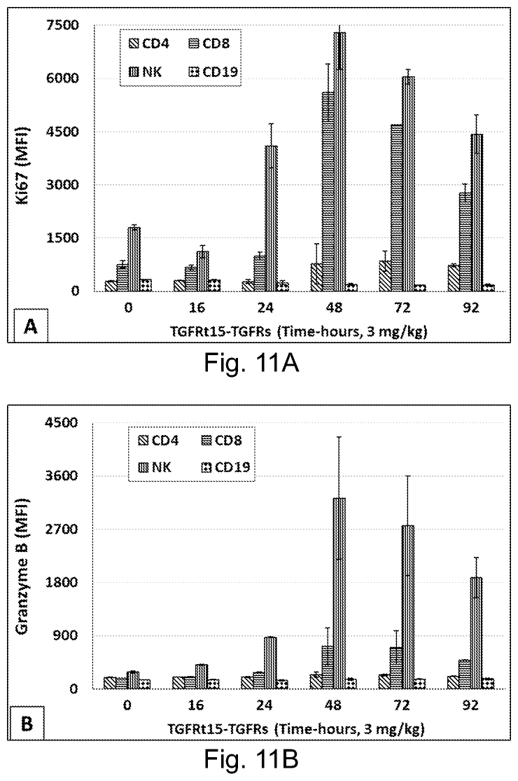

Furthermore, the dynamic proliferation of immune cells based on Ki67 expression of splenocytes and cytotoxicity potential based on granzyme B expression were evaluated in splenocytes isolated from mice following a single dose (3 mg/kg) of TGFRt15-TGFRs. As shown in FIGS. 11A and 11B, in the spleens of mice treated with TGFRt15-TGFRs, the expression of Ki67 and granzyme B by NK cells increased at 24 hours after treatment and its expression of CD8⁺ T cells and NK cells both increased at 48 hours and later time points after the single dose treatment. These results demonstrate that TGFRt15-TGFRs not only increases the numbers of CD8⁺ T cells and NK cells but also enhance the cytotoxicity of these cells. The single dose treatment of TGFRt15-TGFRs led CD8⁺ T cells and NK cells to proliferate for at least 4 days.

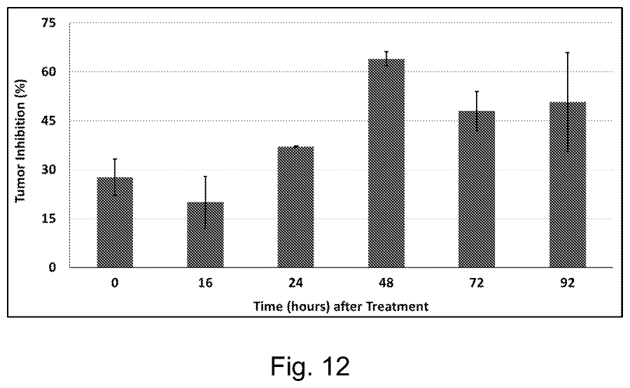

The cytotoxicity of the splenocytes from TGFRt15-TGFRs-treated mice against tumor cells was also evaluated. Mouse Moloney leukemia cells (Yac-1) were labeled with CellTrace Violet and were used as tumor target cells. Splenocytes were prepared from TGFRt15-TGFRs (3 mg/kg)-treated mouse spleens at various time points post treatment and were used as effector cells. The target cells were mixed with effector cells at an E:T ratio=10:1 and incubated at 37° C. for 20 hours. Target cell viability was assessed by analysis of propidium iodide positive, violet-labeled Yac-1 cells using flow cytometry. Percentage of Yac-1 tumor inhibition was calculated using the formula, (1-[viable Yac-1 cell number in experimental sample]/[viable Yac-1 cell number in the sample without splenocytes])×100. As shown in FIG. 12, splenocytes from TGFRt15-TGFRs-treated mice had stronger cytotoxicity against Yac-1 cells than the control mouse splenocytes.

Tumor Size Analysis in Response to Chemotherapy and/or TGFRt15-TGFRs

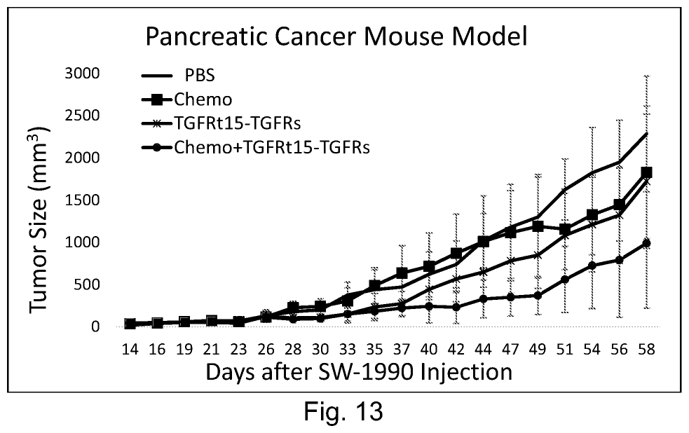

Pancreatic cancer cells (SW1990, ATCC® CRL-2172) were subcutaneously (s.c.) injected into C57BL/6 scid mice (The Jackson Laboratory, 001913, 2×10⁶ cells/mouse, in 100 μL HBSS) to establish the pancreatic cancer mouse model. Two weeks after tumor cell injection, chemotherapy was initiated in these mice intraperitoneally with a combination of Abraxane (Celgene, 68817-134, 5 mg/kg, i.p.) and Gemcitabine (Sigma Aldrich, G6423, 40 mg/kg, i.p.), followed by immunotherapy with TGFRt15-TGFRs (3 mg/kg, s.c.) in 2 days. The procedure above was considered one treatment cycle and was repeated for another 3 cycles (1 cycle/week). Control groups were set up as the SW1990-injected mice that received PBS, chemotherapy (Gemcitabine and Abraxane), or TGFRt15-TGFRs alone. Along with the treatment cycles, tumor size of each animal was measured and recorded every other day, until the termination of the experiment 2 months after the SW1990 cells were injected. Measurement of the tumor volumes were analyzed by group and the results indicated that the animals receiving a combination of chemotherapy and TGFRt15-TGFRs had significantly smaller tumors comparing to the PBS group, whereas neither chemotherapy nor TGFRt15-TGFRs therapy alone work as sufficiently as the combination (FIG. 13).

### In Vitro Senescent B16F10 Melanoma Model

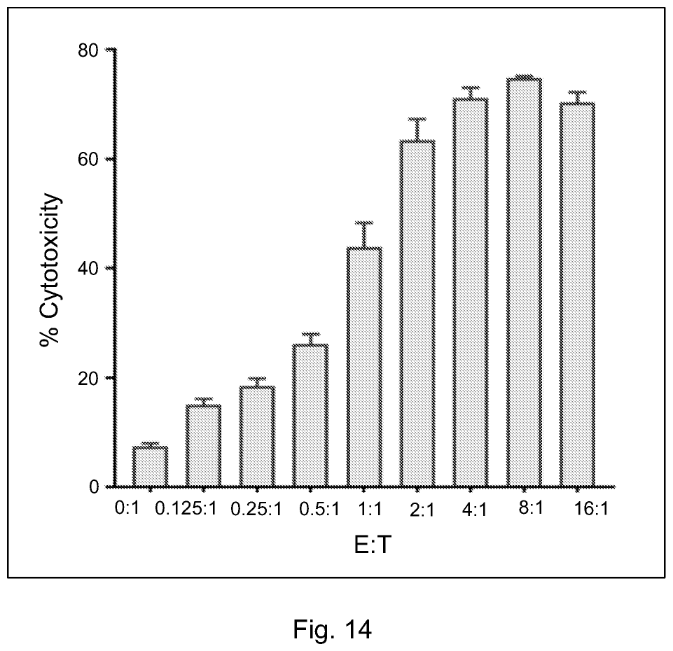

Next, in vitro killing of senescent B16F10 melanoma cells by activated mouse NK cells was evaluated. B16F10 senescence cells (B16F10-SNC) cells were labelled with CellTrace violet and incubated for 16 hrs with different E:T ratio of in vitro 2t2-activated mouse NK cells (isolated from spleen of C57BL/6 mice injected with TGFRt15-TGFRs10 mg/kg for 4 days). The cells were trypsinized, washed and resuspended in complete media containing propidium iodide (PI) solution. The cytotoxicity was assessed by flow cytometry (FIG. 14).

### Example 2: Immunostimulation in C57BL/6 Mice Using a Multi-Chain Polypeptide

### Materials and Methods

An exemplary multi-chain polypeptide (a type A multi-chain polypeptide described herein) was generated that includes a first polypeptide and a second polypeptide, where the first polypeptide is a soluble fusion of two TGFβRII domains, a human tissue factor 219 fragment, and a human IL-15, and the second polypeptide is a soluble fusion of two TGFβRII domains and the sushi domain of human IL-15Rα chain.

### Immunostimulation in C57BL/6 Mice

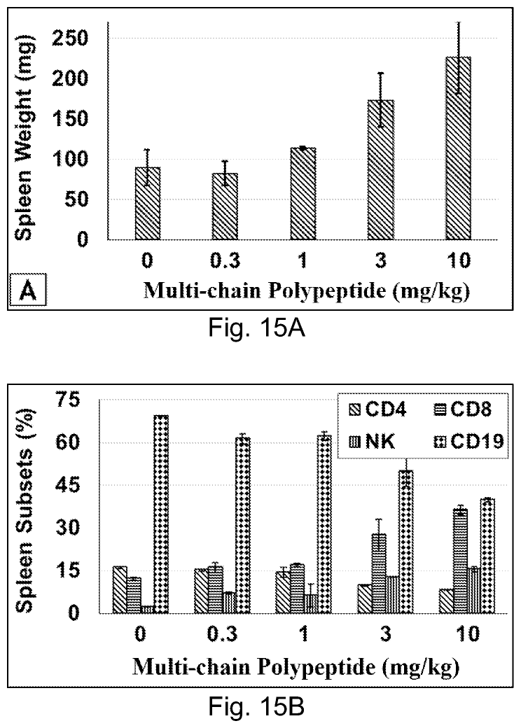

Wild type C57BL/6 mice were treated subcutaneously with either a control PBS solution or with the multi-chain polypeptide at a dosage of 0.3 mg/kg, 1 mg/kg, 3 mg/kg, or 10 mg/kg, respectively. Four days after treatment, spleen weight and the percentages of various immune cell types present in the spleen were evaluated. Specifically, single splenocyte suspensions were generated and stained with fluorochrome-conjugated antibodies including anti-CD4, anti-CD8, anti-NK1.1, and anti-CD19. The percentages of CD4⁺ T cells, CD8⁺ T cells, Natural Killer (NK) cells, and CD19⁺ B cells present in the spleen of mice treated with either the control solution or the multi-chain polypeptide were evaluated using flow cytometry. As shown in FIG. 15A, the spleen weight in mice treated with the multi-chain polypeptide increased with increasing dosage of the multi-chain polypeptide. Moreover, the spleen weight in mice treated with 1 mg/kg, 3 mg/kg, and 10 mg/kg of the multi-chain polypeptide were significantly higher as compared to mice treated with the control solution, respectively. As shown in FIG. 15B, in the spleens of mice treated with the multi-chain polypeptide, the percentages of CD8⁺ T cells and NK cells both increased with increasing dosage of the multi-chain polypeptide. Specifically, the percentages of CD8⁺ T cells were higher in mice treated with 0.3 mg/kg, 3 mg/kg, and 10 mg/kg of the multi-chain polypeptide compared to control-treated mice, and the percentages of NK cells were higher in mice treated with 0.3 mg/kg, 1 mg/kg, 3 mg/kg, and 10 mg/kg of the multi-chain polypeptide compared to control-treated mice. These results demonstrate that the exemplary multi-chain polypeptide is able to stimulate immune cells in the spleen, in particular CD8⁺ T cells and NK cells.

### Pharmacokinetics

The pharmacokinetics of the exemplary multi-chain polypeptide were evaluated in wild type C57BL/6 mice. Mice were treated subcutaneously with the multi-chain polypeptide at a dosage of 3 mg/kg. Blood was collected at various time points via tail vein, and serum was prepared. The concentration of the multi-chain polypeptide in the serum was determined with ELISA. Briefly, the multi-chain polypeptide was captured using an anti-human tissue factor antibody, and detected using a biotinylated anti-human TGFβ receptor, a peroxidase conjugated streptavidin, and ABTS substrate. The results showed that the half-life of the exemplary multi-chain polypeptide was 12.66 hours.

### Immunostimulation Over Time in C57BL/6 Mice

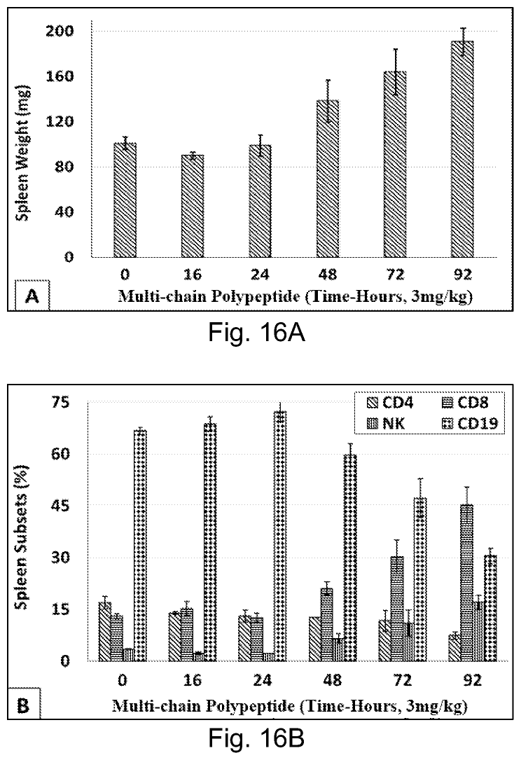

To evaluate the effect of immunostimulation by the multi-chain polypeptide over time, mice were treated with a single dose of the multi-chain polypeptide at 3 mg/kg and the spleen weight and percentages of immune cell types present in the spleen were evaluated immediately upon treatment and at 16, 24, 48, 72, and 92 hours after treatment, using techniques described above. As shown in FIG. 16A, the spleen weight of mice treated with the multi-chain polypeptide increased at 48 hours after treatment, and continued to increase over the next 44 hours. Moreover, as shown in FIG. 16B, in the spleens of mice treated with the multi-chain polypeptide, the percentages of CD8⁺ T cells and NK cells both increased at 48 hours after treatment and continued to increase over the next 44 hours. These results further demonstrate that the exemplary multi-chain polypeptide is able to stimulate immune cells in the spleen, in particular CD8⁺ T cells and NK cells, over time.

### Increased Proliferation and Granzyme B Expression by CD8⁺ T Cells and NK Cells

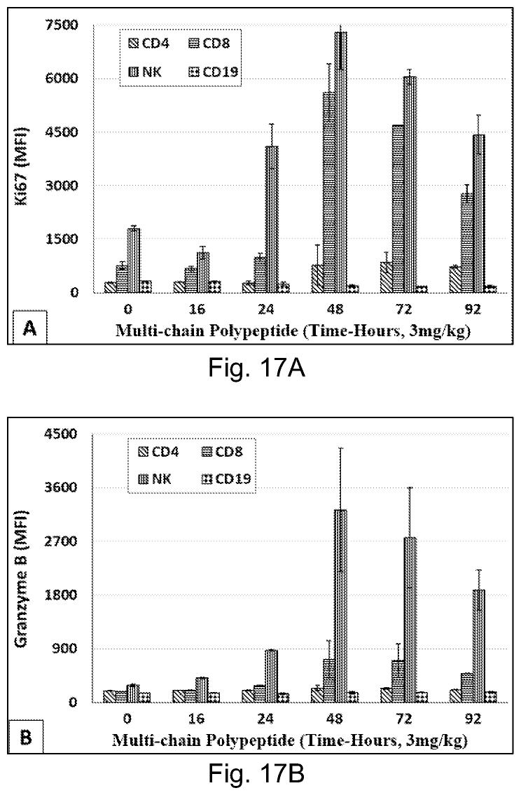

To evaluate the proliferation and cytotoxic potential of the immune cells induced by the multi-chain polypeptide, mice were treated with a single dose of the multi-chain polypeptide at 3 mg/kg, and the spleens of these mice were evaluated immediately after, and at 16, 24, 48, 72, and 92 hours after treatment. Briefly, single splenocyte suspensions were generated and stained with fluorochrome-conjugated antibodies for the various cell types including anti-CD4, anti-CD8, anti-NK1.1, and anti-CD19, and with an anti-Ki67 antibody (i.e. a cell proliferation marker) and an anti-Granzyme B antibody (i.e. a cytotoxic marker). The mean fluorescent intensity (MFI) of Ki67 and Granzyme B for each immune cell type was analyzed by flow cytometry. As shown in FIGS. 17A and 17B, the expression of Ki67 and Granzyme B by NK cells showed an increase at 24 hours as well as each time point evaluated thereafter as compared to immediately after treatment (0 hours). Moreover, the expression of Ki67 and Granzyme B by CD8⁺ T cells showed an increase at 48 hours as well as each time point evaluated thereafter as compared to immediately after treatment (0 hours). As such, a single dose of the multi-chain polypeptide resulted in proliferation of CD8⁺ T cells and NK cells for up to at least 4 days post-treatment.

These results demonstrate that the multi-chain polypeptide not only increased the number of CD8⁺ T cells and NK cells in the spleen, but also enhanced the proliferation and cytotoxicity of these cells.

### Cytotoxicity Against Tumor Cells

Next, the cytotoxicity of the splenocytes activated by the multi-chain polypeptide against tumor cells were evaluated in C57BL/6 mice. Mouse Moloney leukemia cells (Yac-1) were labeled with CellTrace Violet and used as tumor target cells. C57BL/6 mice were treated with a single dose of the multi-chain polypeptide at 3 mg/kg, and splenocytes were prepared at various time points thereafter and used as effector cells. The target tumor cells were mixed with the effector cells at an effector:target (E:T) ratio of 10:1, and incubated at 37° C. for 20 hours. Target cell viability was assessed by analyzing Propidium Iodide (PI)-positive, violet-labeled Yac-1 cells using flow cytometry. The percentage of Yac-1 tumor inhibition was calculated using the formula:

Percentage of Yac-1 tumor inhibition=(1−viable Yac-1 cell number in experimental sample/viable Yac-1 cell number in the sample without splenocytes)×100

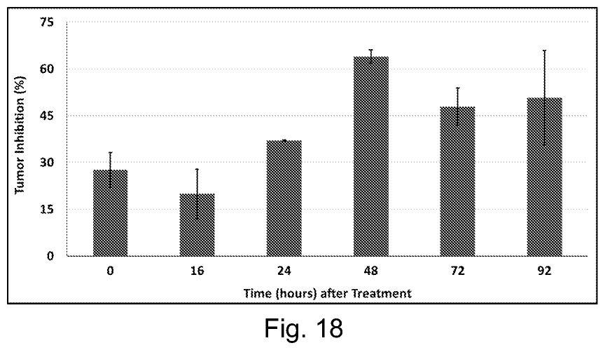

As shown in FIG. 18, splenocytes from mice after 24-hour or more treatment with the multi-chain polypeptide showed increased cytotoxicity against Yac-1 cells as compared to the splenocytes from untreated mice.

### Example 3: Immunostimulation in C57BL/6 Mice Using a High Fat Diet-Based Type-2 Diabetes Mouse Model

### Materials and Methods

TGFRt15-TGFRs is a multi-chain chimeric polypeptide (a type A multi-chain chimeric polypeptide described herein) that includes two TGFβ-binding domains which a soluble human TGFβRII dimer (aa24-159). 21t15-TGFRs is a multi-chain chimeric polypeptide (a type A multi-chain chimeric polypeptide described herein) that includes IL-21 and a TGFβ-binding domain. 2t2 is a chimeric polypeptide (a type B chimeric polypeptide described herein) that include two IL-2 polypeptides.

### Results

To evaluate the effect of TGFRt15-TGFRs, 2t2, and 21t15-TGFRs in treating Type-2 diabetes, a high fat diet-based Type-2 diabetes mouse model (B6.129P2-ApoE^{tm1Unc}/J from The Jackson Laboratory) was used. Mice were fed either a control diet or a high fat diet for 11 weeks. A subset of mice fed with the high fat diet were also treated with TGFRt15-TGFRs, 2t2, or 21t15-TGFRs. Mice fed the control diet, high fat diet, and mice fed with the high fat diet and treated with TGFRt15-TGFRs, 2t2, or 21t15-TGFRs were evaluated 4 days post-treatment. Briefly, single splenocyte suspensions were generated and stained with fluorochrome-conjugated antibodies including anti-CD4, anti-CD8, anti-NK1.1, and anti-CD19. The percentages of CD4⁺ T cells, CD8⁺ T cells, Natural Killer (NK) cells, and CD19⁺ B cells present in the spleen of mice in each group were evaluated using flow cytometry.

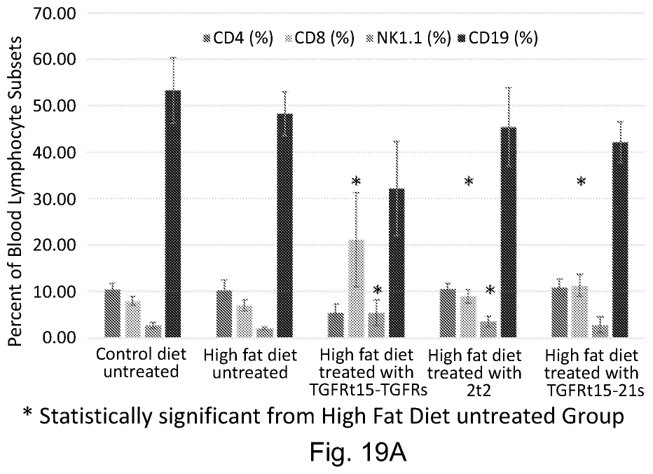

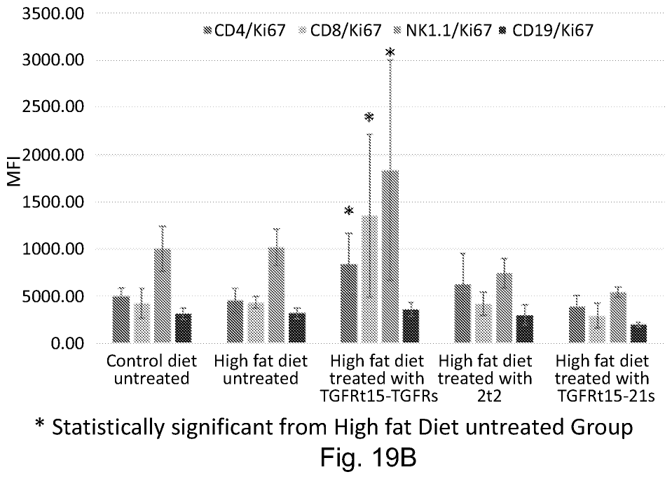

As shown in FIG. 19A, in mice fed a high fat diet, the percentage of NK cells in PBMCs was significantly increased after treatment with TGFRt15-TGFRs or 2t2 compared to untreated mice, but not after treatment with 21t15-TGFRs. Furthermore, the percentage of CD8⁺ T cells in PBMCs was significantly increased after treatment with TGFRt15-TGFRs, 2t2, or 21t15-TGFRs compared to untreated mice. Moreover, the proliferation of CD4⁺ T cells, CD8⁺ T cells, Natural Killer (NK) cells, and CD19⁺ B cells in PBMCs were also evaluated using an anti-Ki67 antibody. As shown in FIG. 19B, the number of proliferating NK cells, CD4⁺ T cells, and CD8⁺ T cells were significantly increased after treatment with TGFRt15-TGFRs, but not after treatment with 2t2 or 21t15-TGFRs.

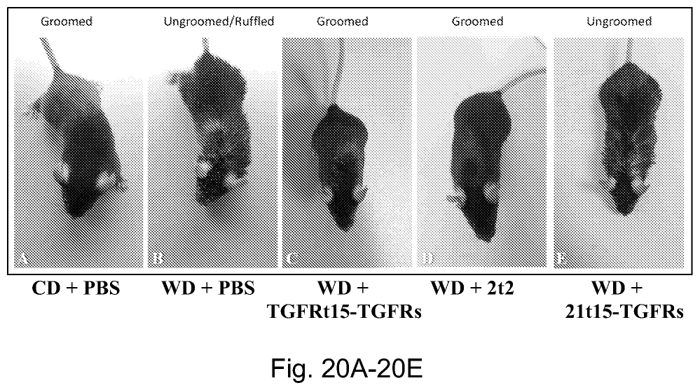

To examine the effect of TGFRt15-TGFRs, 2t2 and 21t15-TGFRs on the appearance and texture of skin and hair in animals, mice were fed either a control or a high fat diet for 7 weeks, and a subset of the mice fed a high fat diet were also treated with TGFRt15-TGFRs, 2t2 or 21t15-TGFRs. One week post-treatment, the appearance of the mice was evaluated. Mice fed a high fat diet and untreated, or a high diet and treated with 21t15-TGFRs appeared ungroomed and ruffled, and had increased gray hair/hair loss as compared to mice fed a control diet (FIGS. 20A, 20B and 20E). Surprisingly, mice fed a high fat diet that received TGFRt15-TGFRs or 2t2 treatment appeared groomed and healthier (less gray hair/hair loss) (FIGS. 20C and 20D) as compared to mice fed a high fat diet that did not receive TGFRt15-TGFRs or 2t2 treatment (FIG. 20B). Specifically, TGFRt15-TGFRs or 2t2-treated mice showed superior skin and hair appearance and texture as compared to control mice. These results demonstrate that treatment with TGFRt15-TGFRs or 2t2 improves the appearance and texture of skin and hair in mammals.

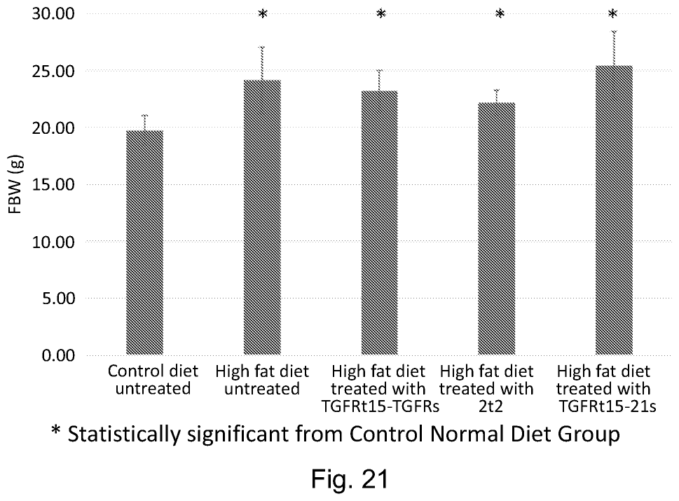

Next, mice were fed either a control or high fat diet for 9 weeks, and a subset of the mice fed a high fat diet were treated with TGFRt15-TGFRs, 2t2, or 21t15-TGFRs. Four days post-treatment, the fasting body weight of mice in each group were measured. The fasting body weight of mice fed with the high fat diet and untreated, as well as mice fed with the high fat diet and treated with 21t15-TGFRs were significantly increased compared to mice fed a control diet. However, the fasting body weight of mice fed a high fat diet and treated with TGFRt15-TGFRs or 2t2 were decreased compared to the other two high fat diet groups mentioned above. The fasting body weight of the mice at the end of the study (9 weeks) is shown in FIG. 21.

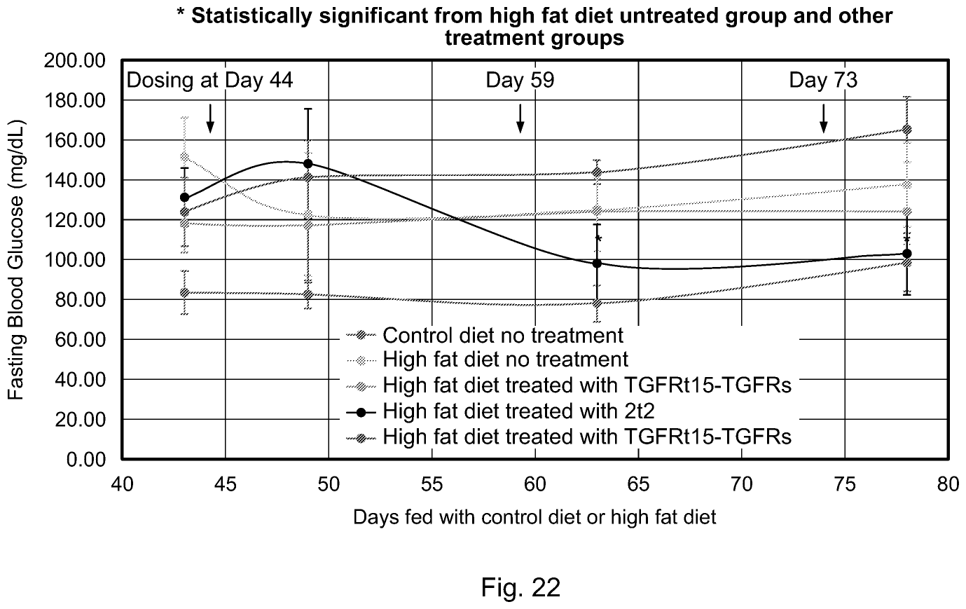

To evaluate the fasting glucose levels in the mice of each group, mice were fed either a control or a high fat diet and were either untreated or treated with TGFRt15-TGFRs, 2t2, or 21t15-TGFRs on days 44, 59 and 73. The fasting blood glucose in the mice of each group were measured 4 days post-treatment. As shown in FIG. 22, after the second and third doses (on Days 59 and 73, respectively), the fasting blood glucose level was significantly reduced for mice fed a high fat diet and treated with 2t2 (red line) as compared to mice fed a high fat diet but untreated (yellow line). The fasting blood glucose level remained constant for mice fed a high fat diet and treated with TGFRt15-TGFRs (green line), whereas the fasting blood glucose level increased for mice fed a high fat diet and treated with 21t15-TGFRs (blue line).

### Example 4: Chemotherapy-Induced Senescent B16F10 Melanoma Cells Express NK Ligands

### Material and Methods

Cellular senescence in B16F10 melanoma cells was induced by treating the cells with docetaxel (7.5 μM, Sigma) for 3 days followed by recovery in complete media for 4 days. Cellular senescence was accessed by staining the cells with senescence-associated β-galactosidase (SA β-gal). Briefly, B16F10 control and senescence cells (B16F10-SNC) were washed once with PBS, fixed with 0.5% glutaraldehyde (PBS (pH 7.2)), for 30 minutes. Cells were stained in X-gal solution (1 mg/mL X-gal, 0.12 mM K3Fe [CN]₆, 0.12 mM K4Fe[CN]₆, and 1 mM MgCl₂ in PBS at pH 6.0) overnight at 37° C., and were imaged using a Nikon optical light microscope.

### Results

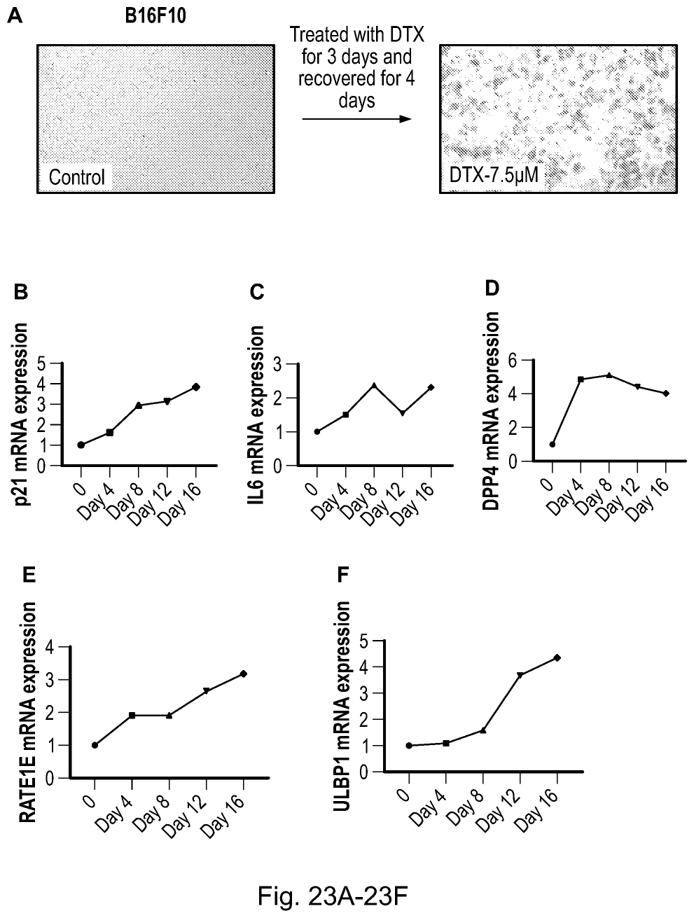

Cellular senescence in B16F10 melanoma cells was induced using chemotherapy as described above. As shown in FIG. 23A, chemotherapy-induced senescent B16F10 cells (B16F10-SNC) were positive for SA β-gal staining, while the control B16F10 cells were not stained. Next, expression of senescence genes was analyzed using RT-qPCR with RNA isolated on day 0 or following senescence induction on days 4, 8, 12 and 16, respectively. The expression levels were normalized to control B16F10 cells. As shown in FIGS. 23B-23D, the expression of p21, IL6 and DPP4 were upregulated in RNA isolated from the senescent cells over the duration of the experiment. Moreover, as shown in FIGS. 23E and 23F, the expression of RATE1E and ULBP1 (NK activating receptor NKG2D ligands) were also induced in senescent cells, with the highest expression level being on day 16. These results demonstrate that the chemotherapy-induced senescent B16F10 cells are subjected to stronger cytotoxicity of activated NK cells than control B16F10 cells.

### Acquisition of Stem-cell Properties in Chemotherapy-induced Senescent B16F10 Melanoma Cells

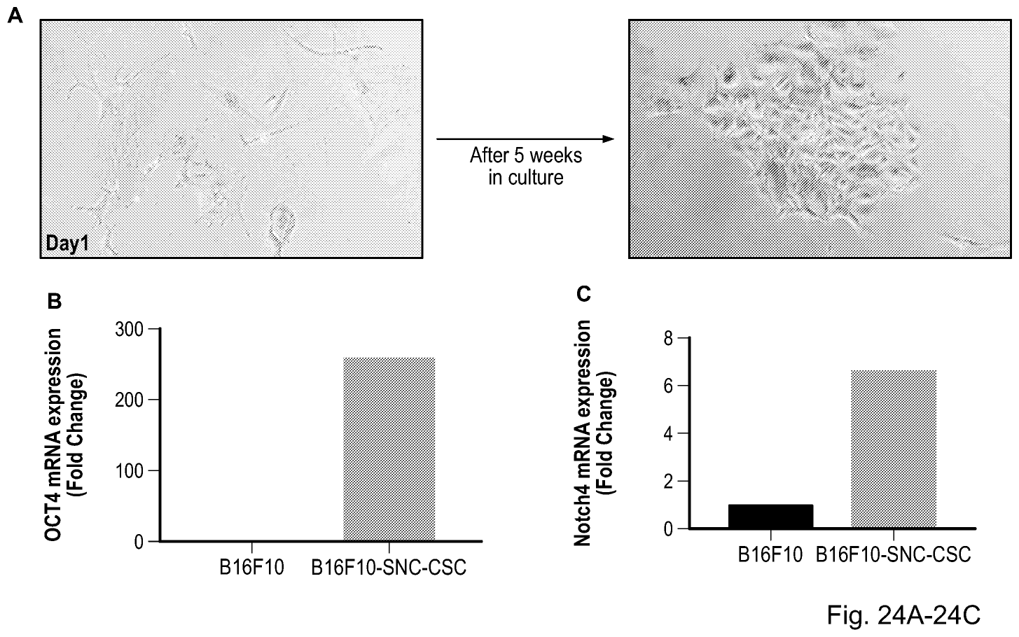

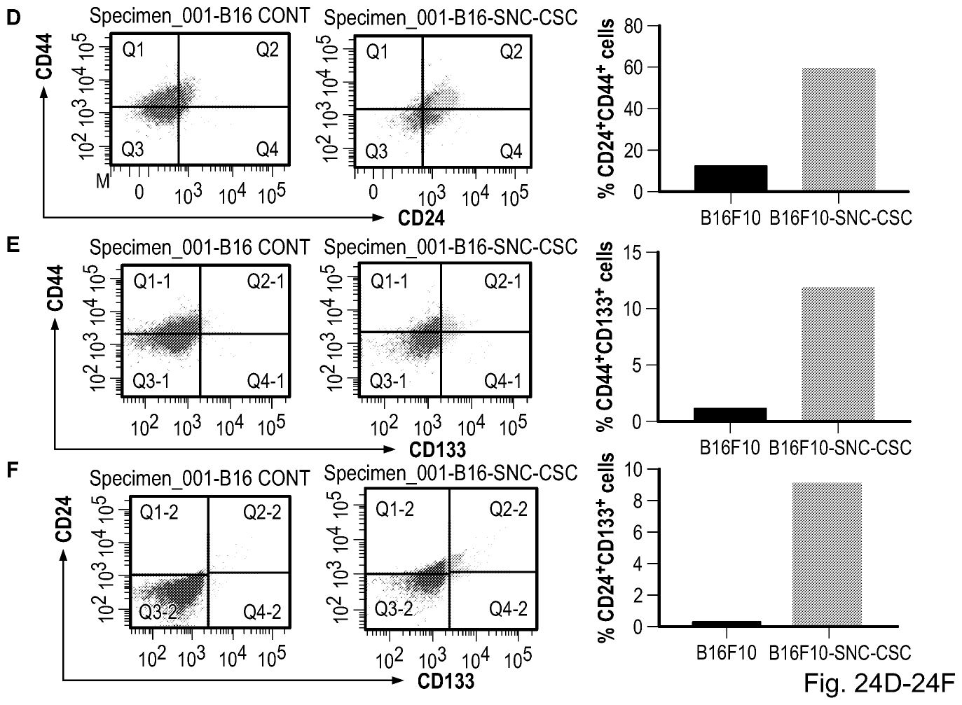

To examine whether chemotherapy-induced senescent B16F10 melanoma cells acquired stem cell properties, a colony formation assay was performed. Briefly, 1000 cells/well were seeded on a six well plate, and the media was changed every third day. As shown in FIG. 24A (images taken at 100× magnification), after 5 weeks in culture the senescent cells were able to form colonies. To evaluate stem cell marker expression by the colonies, RNA was isolated from the colonies and the expression of Oct4 and Notch4 mRNA were determined by RT-qPCR. As compared to control B16F10 cells, chemotherapy-induced senescent B16F10 melanoma cells showed upregulation of Oct4 and Notch 4, which are cancer stem cell markers (FIGS. 24B and 24C). Moreover, cell surface expression of stem cell markers CD44, CD24 and CD133 were evaluated by staining with antibodies against CD44, CD24, and CD133 followed by flow cytometry. As shown in FIGS. 24D-24F, double positive populations (CD44 CD24+, CD44+CD133+, and CD24+CD133+) were increased in the chemotherapy induced senescence stem cells (B16F10-SNC-CSC) compared to control B16F10.

Chemotherapy-Induced Senescent (CIS) Melanoma Cells with Stem Cell Properties are More “Migratory” and “Invasive” than Control B16F10 Cells

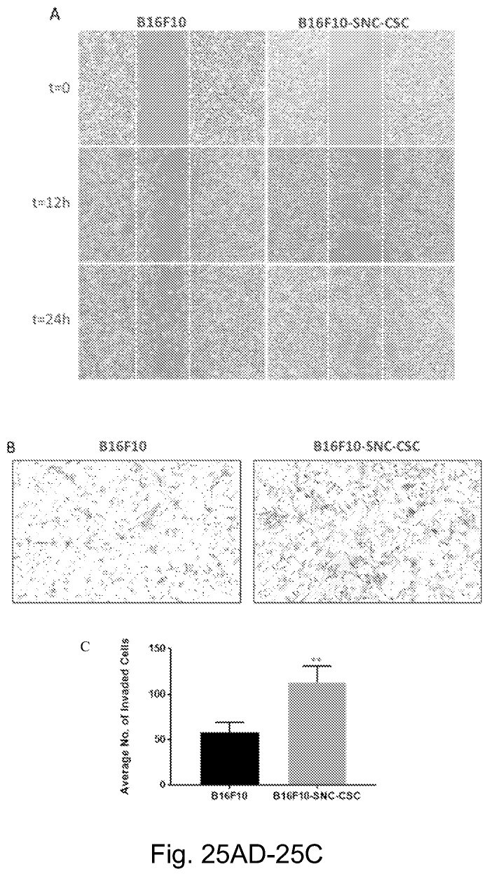

The migratory properties of chemotherapy-induced senescent (CIS) melanoma cells with stem cell properties (B16F10-SNC-CSC) were analyzed using a migration assay. Briefly, control B16F10 cells and B16F10-SNC-CSC cells were plated on six well plates and wounded with a p20 pipette tip. Movement of cells were imaged at 0, 12, and 24 hours after. As shown in FIG. 25A, chemotherapy-induced senescent (CIS) melanoma cells with stem cell properties (B16F10-SNC-CSC) were more migratory in the in vitro migration assay, as compared to control B16F10 cells.

Next, the invasive properties of chemotherapy-induced senescent cells with stem cell properties (B16F10-SNC-CSC) were analyzed using an invasion assay. The invasion 6 assay was carried out on 24-well transwell inserts coated with Matrigel. Briefly, 0.5×10⁶ control B16F10 cells and B16F10-SNC-CSC cells were seeded in serum-free media onto the upper chamber, and the lower chamber was filled with media supplemented with 10% FBS. After 16 hours of incubation, the cells on the upper surface of the filter were removed, and cells underneath the filter were fixed and stained with a 0.02% crystal violet solution. The number of cells were counted in three fields at 100× magnification. As shown in FIGS. 25B and 25C, chemotherapy-induced senescent cells with stem cell properties were more aggressive in invading the Matrigel coated membrane as compared to control B16F10 cells. These results demonstrate that chemotherapy-induced senescent B16F10 tumor cells are able to regain their proliferation capability, obtain stem-cell features, and have increased migratory abilities and invasiveness for metastasis.

Cytotoxic Activity of Mouse NK Cells on Chemotherapy-Induced Senescent Cells with Stem Cell Properties

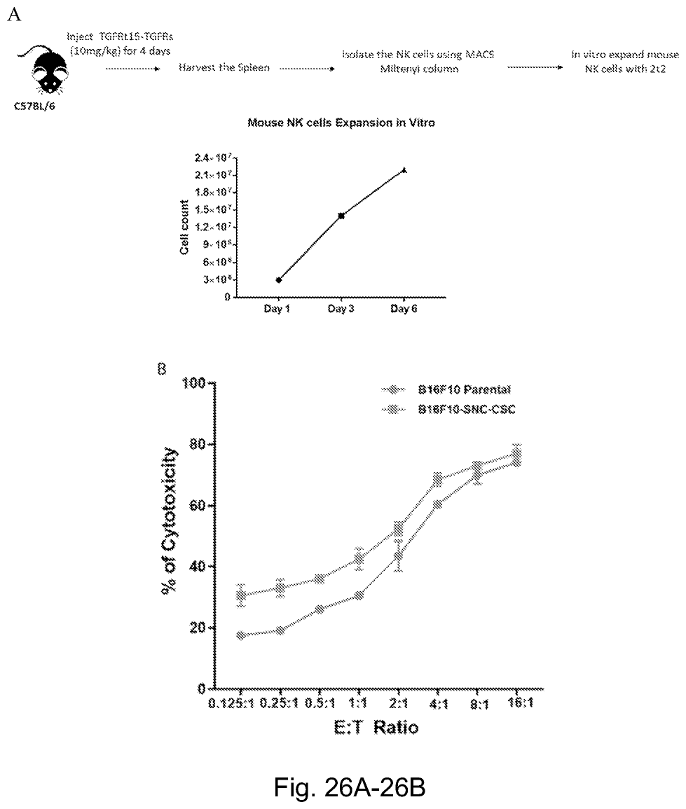

To expand NK cells in vivo, C57BL/6 mice were injected subcutaneously with TGFRt15-TGFRs (10 mg/kg) for 4 days. The spleens from these mice were obtained and NK cells were purified using MACS Miltenyi column. The purified NK cells were then expanded in vitro with 2t2 (FIG. 26A).

To evaluate the cytotoxicity of the expanded NK cells, chemotherapy-induced senescent stem cells (B16F10-SNC-CSC) or control B16F10 cells were labelled with CellTrace violet and incubated with in vitro activated 2t2 mouse NK cells (isolated from spleen of C57BL/6 mice injected with 10 mg/kg TGFRt15-TGFRs for 4 days) at various E:T ratios for 16 hrs. The B16F10-SNC-CSC and control B16F10 cells were trypsinized, washed and re-suspended in complete media containing a Propidium Iodide (PI) solution, and cytotoxicity was accessed by flow cytometry. As shown in FIG. 26B, NK cells were more effective at killing chemotherapy-induced senescent cells with stem cell properties (B16F10-SNC-CSC), as compared to control B16F10 cells.

### Combination Treatment in Melanoma Mouse Model

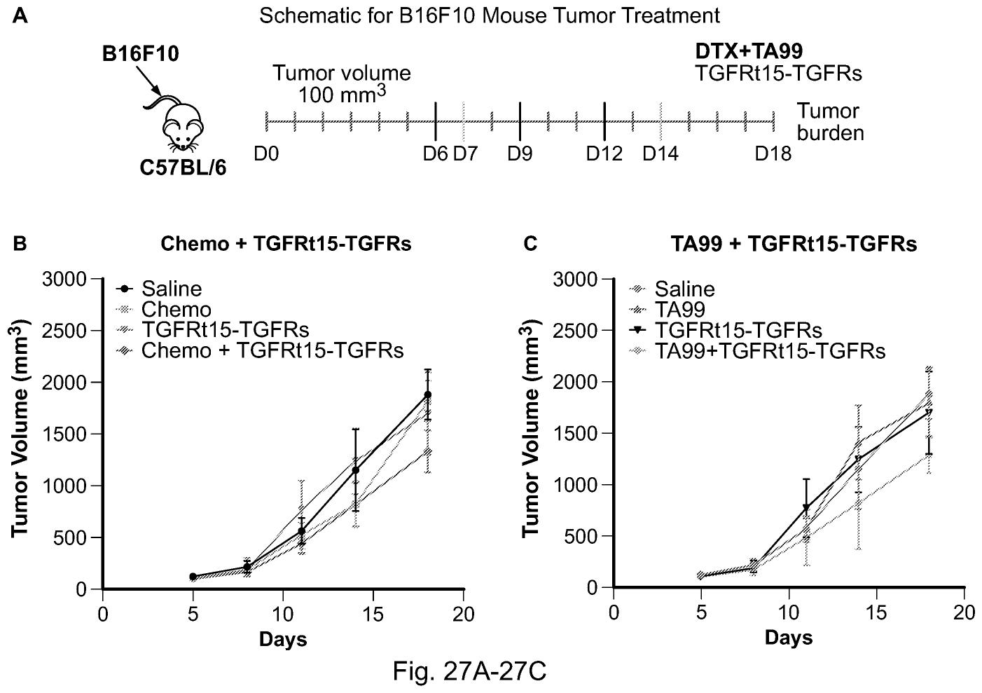

The effect of TGFRt15-TGFRs in treating melanoma was evaluated in a mouse melanoma model. Briefly, 5×10⁵ B16F10 cells were injected subcutaneously into C57BL/6 mice. When the tumor volume reached ˜100 mm³, mice were treated with docetaxel (chemotherapy) (5 mg/kg) or TA99 (200 μg) either as a single agent or in combination every third day, and TGFRt15-TGFRs (3 mg/kg) was given once a week (FIG. 27A). Mice that received saline, docetaxel (chemotherapy)/TA99 alone, or TGFRt15-TGFRs alone were used as controls. Five mice were tested in each experimental and control group. Tumor volume was measured every third day. As shown in FIGS. 27B and 27C, combinations of TGFRt15-TGFRs with either chemotherapy or TA99 slowed down tumor progression as compared to mice treated with saline or mice treated with chemotherapy or TA99 alone in the syngeneic melanoma mouse model.

### Example 5: Stimulation of NK Cells In Vivo by 212 and/or TGFRt15-TGFRs

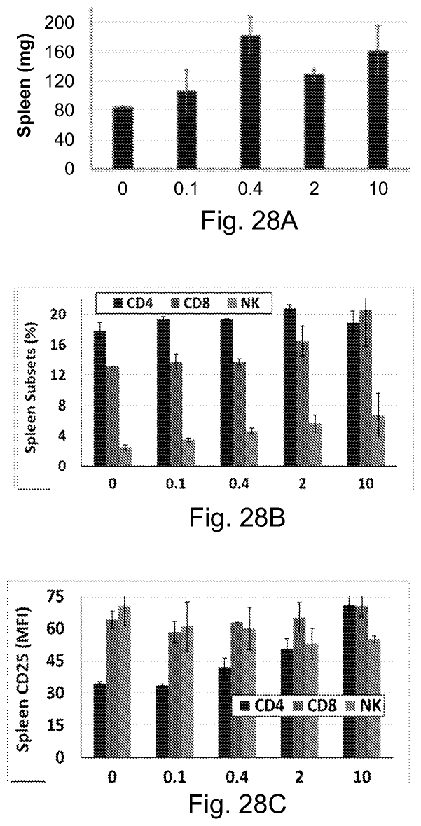

A set of experiments was performed to determine the effect of the 2t2 construct on immune stimulation in C57BL/6 mice. In these experiments, C57BL/6 mice were subcutaneously treated with control solution (PBS) or 2t2 at 0.1, 0.4, 2, and 10 mg/kg. Treated mice were euthanized 3 days post-treatment. Spleen weight was measured and single splenocyte suspensions were prepared. Splenocytes suspensions were stained with conjugated anti-CD4, anti-CD8, and anti-NK1.1 (NK) antibodies. The percentage of CD4⁺ T cells, CD8⁺ T cells, and NK cells, and CD25 expression on lymphocyte subsets were analyzed by flow cytometry. FIG. 28A shows that 212 was effective at expanding splenocytes based on spleen weight especially at a dose level of 0.1-10 mg/kg. Following treatment, the percentage of CD8⁺ T cells were higher in 2t2-treated mice compared to control-treated mice at 2 and 10 mg/kg (FIG. 28B). The percentage of NK cells were also higher in 2t2-treated mice compared to control-treated mice at all doses of 2t2 tested (FIG. 28B). Additionally, 2t2 significantly upregulated CD25 expression by CD4⁺ T cells, but not CD8⁺ T cells and NK cells following treatment at 0.4 to 10 mg/kg (FIG. 28C).

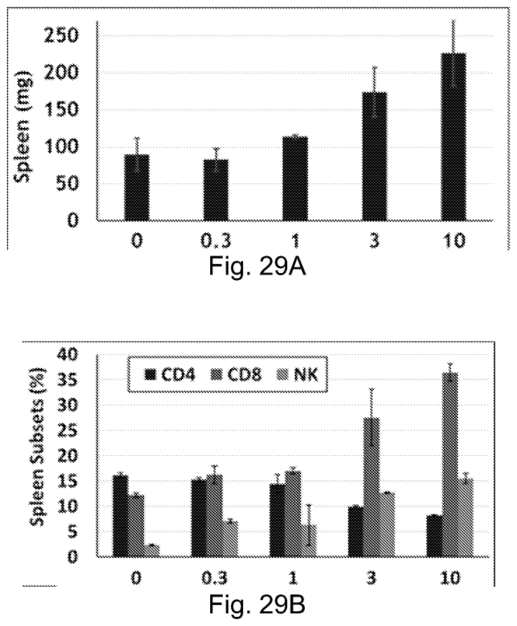

A set of experiments was performed to determine the effect of the TGFRt15-TGFRs construct on immune stimulation in C57BL/6 mice. In these experiments, C57BL/6 mice were subcutaneously treated with control solution (PBS) or TGFRt15-TGFRs at 0.3, 1, 3, and 10 mg/kg. The treated mice were euthanized 4 days post-treatment. Spleen weight was measured and single splenocyte suspensions were prepared. The splenocytes suspensions were stained with conjugated anti-CD4, anti-CD8, and anti-NK1.1 (NK) antibodies. The percentage of CD4⁺ T cells, CD8⁺ T cells, and NK cells were analyzed by flow cytometry. FIG. 29A shows that spleen weight in mice treated with TGFRt15-TGFRs increased with increasing dosage of TGFRt15-TGFRs. Additionally, spleen weight in mice treated with 1 mg/kg, 3 mg/kg, and 10 mg/kg of TGFRt15-TGFRs were higher as compared to mice treated with the control solution. FIG. 29B shows that the percentages of CD8⁺ T cells and NK cells both increased with increasing dosage of TGFRt15-TGFRs. Specifically, the percentages of CD8⁺ T cells were higher in mice treated with 0.3 mg/kg, 3 mg/kg, and 10 mg/kg of TGFRt15-TGFRs compared to control-treated mice, and the percentages of NK cells were higher in mice treated with 0.3 mg/kg, 1 mg/kg, 3 mg/kg, and 10 mg/kg of TGFRt15-TGFRs compared to control-treated mice.

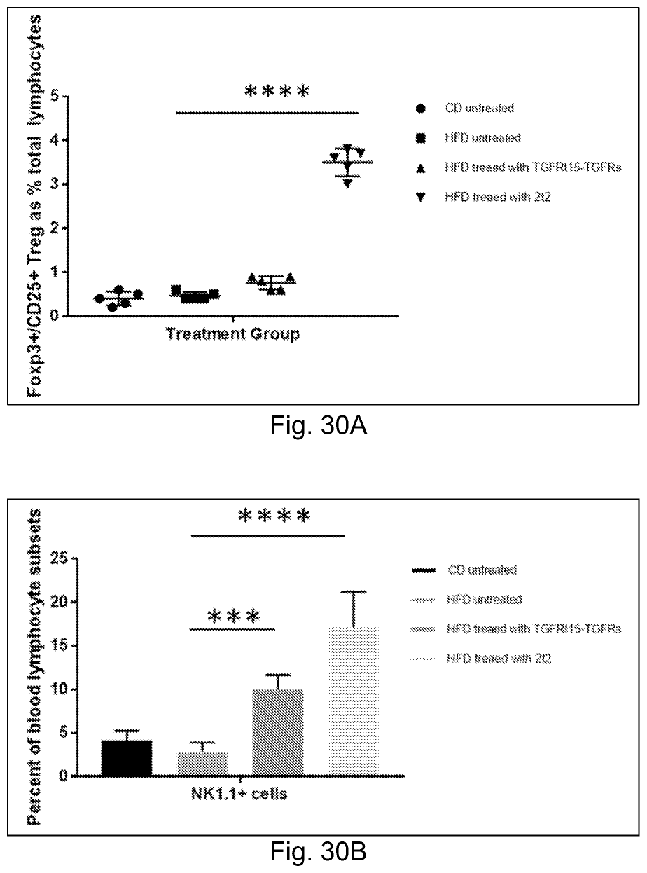

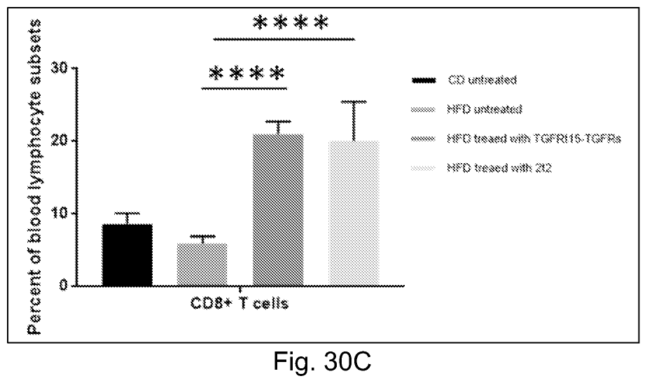

A set of experiments was performed to determine the effect of the TGFRt15-TGFRs construct or 2t2 construct on immune stimulation in ApoE^{-/-} mice fed with a Western diet. In these experiments, 6-week old female B6.129P2-ApoE^{tm1Unc}/J mice (Jackson Laboratory) were fed with a Western diet containing 21% fat, 0.15% cholesterol, 34.1% sucrose, 19.5% casein, and 15% starch (TD88137, Envigo Laboratories). After 8-weeks of the Western diet, the mice were injected subcutaneously with TGFRt15-TGFRs or 2t2 at 3 mg/kg. Three days post treatment, mice were fasted for 16 hours and then blood samples were collected through retro-orbital venous plexus puncture. The blood was mixed with 10 μL 0.5 M EDTA, and 20 μL blood was taken for lymphocyte subsets analysis. The red blood cells were lysed with ACK (0.15 M NH₄Cl, 1.0 mM KHCO₃, 0.1 mM Na₂EDTA, pH 7.4) and the lymphocytes were stained with anti-mouse CD8a and anti-mouse NK1.1 antibodies for 30 minutes at 4° C. in FACS staining buffer (1% BSA in PBS). The cells were washed once and analyzed with a BD FACS Celesta. For Treg staining, ACK treated blood lymphocytes were stained with anti-mouse CD4 and anti-mouse CD25 antibodies for 30 minutes at 4° C. in FACS staining buffer. The cells were washed once and resuspended in fixation/permeabilization working solution and incubated at room temperature for 60 minutes. The cells were washed once and resuspended in permeabilization buffer. The samples were centrifuged at 300-400×g for 5 minutes at room temperature and the supernatant was then discarded. The cell pellet was resuspended in residual volume and the volume adjusted to about 100 μL with 1× permeabilization buffer. Anti-Foxp3 antibody was added to the cells, and the cells were incubated for 30 minutes at room temperature. Permeabilization buffer (200 μL) was added to the cells, and the cells were centrifuged at 300-400×g for 5 minutes at room temperature. The cells were resuspended in flow cytometry staining buffer and analyzed on a flow cytometer. FIGS. 30B-30C show that treatment with TGFRt15-TGFRs and 2t2 increased the percentage of NK cells and CD8⁺ T cells in ApoE^{-/-} mice fed with Western diet. FIG. 30A shows that treatment with 212 also increased the percentage of Treg cells.

### Example 6: Induction of Proliferation of Immune Cells In Vivo

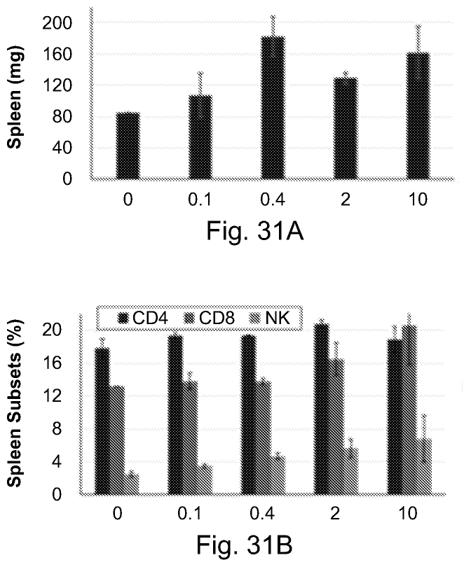

A set of experiments was performed to determine the effect of the 2t2 construct on immune cell stimulation in C57BL/6 mice. In these experiments, C57BL/6 mice were subcutaneously treated with control solution (PBS) or 2t2 at 0.1, 0.4, 2, and 10 mg/kg. Treated mice were euthanized 3 days post-treatment. Spleen weight was measured and single splenocyte suspensions were prepared. The splenocyte suspensions were stained with conjugated anti-CD4, anti-CD8, and anti-NK1.1 (NK) antibodies. The percentage of CD4⁺ T cells, CD8⁺ T cells, and NK cells were analyzed by flow cytometry. FIG. 31A shows that 2t2 treatment was effective at expanding splenocytes based on spleen weight especially at 0.1-10 mg/kg. The percentage of CD8⁺ T cells was higher compared to control-treated mice at 2 and 10 mg/kg (FIG. 31B). Additionally, the percentage of NK cells was higher compared to control-treated mice at all doses of 2t2 tested (FIG. 31B). These results demonstrate that 2t2 treatment was able to induce proliferation of CD8⁺ T cells and NK cells in C57BL/6 mice.

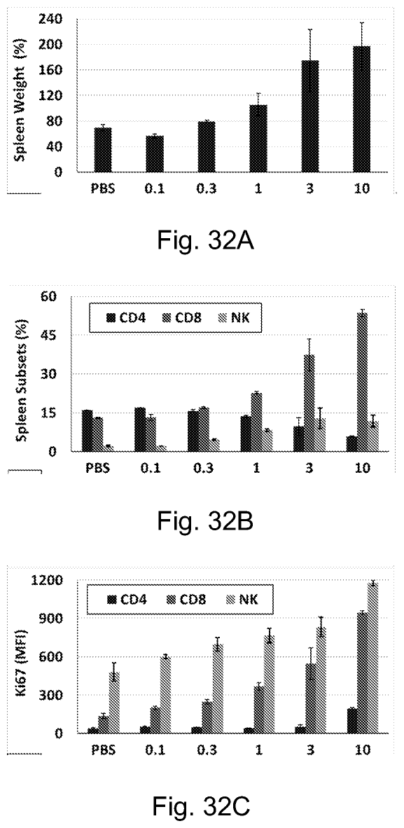

A set of experiments was performed to determine the effect of the TGFRt15-TGFRs construct on immune stimulation in C57BL/6 mice. In these experiments, C57BL/6 mice were subcutaneously treated with control solution (PBS) or TGFRt15-TGFRs at 0.1, 0.3, 1, 3, and 10 mg/kg. The treated mice were euthanized 4 days post-treatment. Spleen weight was measured and splenocyte suspensions were prepared. The splenocyte suspensions were stained with conjugated anti-CD4, anti-CD8, and anti-NK1.1 (NK) antibodies. The cells were additionally stained for proliferation marker Ki67. FIG. 32A shows that spleen weight in mice treated with TGFRt15-TGFRs increased with increasing dosage of TGFRt15-TGFRs. Additionally, spleen weight in mice treated with 1 mg/kg, 3 mg/kg, and 10 mg/kg of TGFRt15-TGFRs was higher as compared to mice treated with just the control solution. The percentages of CD8⁺ T cells and NK cells both increased with increasing dosage of TGFRt15-TGFRs (FIG. 32B). Finally, TGFRt15-TGFRs significantly upregulated expression of cell proliferation marker Ki67 in both CD8⁺ T cells and NK cells at all doses of TGFRt15-TGFRs tested. These results demonstrate that TGFRt15-TGFRs treatment induced proliferation of both CD8⁺ T cells and NK cells in C57BL/6 mice.

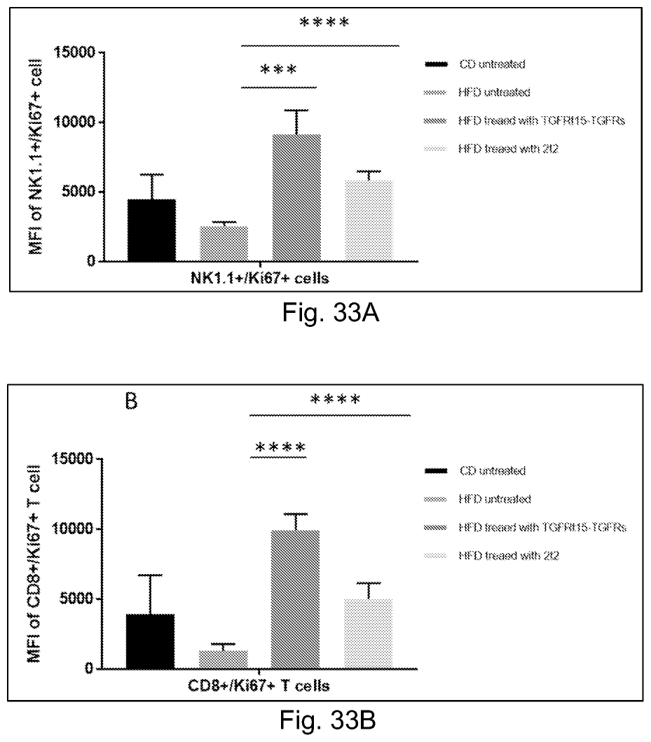

A set of experiments was performed to determine the effect of the TGFRt15-TGFRs construct or the 2t2 construct on immune stimulation in ApoE^{-/-} mice fed with a Western diet. In these experiments, 6-week old female B6.129P2-ApoE^{tm1Unc}/J mice (Jackson Laboratory) were fed with a Western diet containing 21% fat, 0.15% cholesterol, 34.1% sucrose, 19.5% casein, and 15% starch (TD88137, Envigo Laboratories). After 8-week of the Western diet, the mice were injected subcutaneously with TGFRt15-TGFRs or 2t2 at 3 mg/kg. Three days post-treatment, the mice were fasted for 16 hours and then blood samples were collected through retro-orbital venous plexus puncture. The blood was mixed with 10 μL 0.5 M EDTA and 20 μL blood was taken for lymphocyte subsets analysis. The red blood cells were lysed with ACK (0.15 M NH4C1, 1.0 mM KHCO₃, 0.1 mM Na₂EDTA, pH 7.4) and the lymphocytes were stained with anti-mouse CD8a and anti-mouse NK1.1 antibodies for 30 minutes at 4° C. in FACS staining buffer (1% BSA in PBS). The cells were washed once and resuspended in Fixation Buffer (BioLegend Cat #420801) for 20 minutes at room temperature. The cells were centrifuged at 350×g for 5 minutes, the fixed cells were resuspended in Intracellular Staining Permeabilization Wash Buffer (BioLegend Cat #421002) and then centrifuged at 350×g for 5 minutes. The cells were then stained with anti-Ki67 antibody for 20 minutes at RT. The cells were washed twice with Intracellular Staining Permeabilization Wash Buffer and centrifuged at 350×g for 5 minutes. The cells were then resuspended in FACS staining buffer. Lymphocyte subsets were analyzed with a BD FACS Celesta. As described in FIG. 33A, treatment of ApoE^{-/-} mice with TGFRt15-TGFRs induced proliferation (Ki67-positive staining) in NK and CD8⁺ T cells. Additionally, FIG. 33B shows treatment of ApoE^{-/-} mice with 2t2 also induced proliferation (Ki67-positive staining) in NK and CD8⁺ T cells.

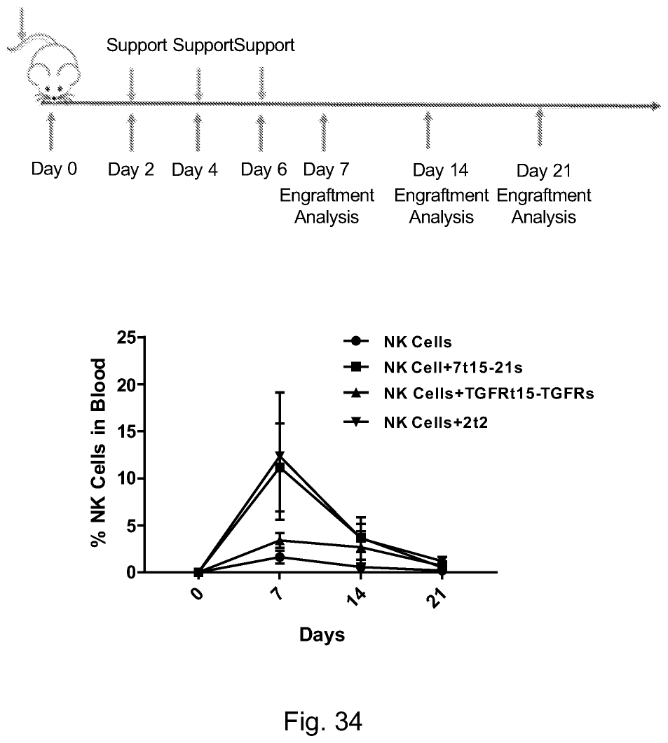

A set of experiments was performed to determine the effect 7t15-21s+anti-TF antibody-expanded NK cells in NSG mice following treatment with 7t15-21s, TGFRt15-TGFRs, and 2t2. In these experiments, fresh human leukocytes were obtained from the blood bank and CD56+NK cells were isolated with the RosetteSep/human NK cell reagent (StemCell Technologies). The purity of NK cells was >90% and confirmed by staining with CD56-BV421, CD16-BV510, CD25-PE, and CD69-APCFire750 antibodies (BioLegend). The cells were counted and resuspended in 2×10⁶/mL in a 24-well flat-bottom plate in 2 mL of complete media (RPMI 1640 (Gibco) supplemented with 2 mM L-glutamine (Thermo Life Technologies), penicillin (Thermo Life Technologies), streptomycin (Thermo Life Technologies), and 10% FBS (Hyclone)). The cells were stimulated with: 7t15-21s (100 nM) and anti-TF antibody (50 nM) for 15 days. After every 2 days, the cells were resuspended at 2×10⁶/mL with fresh media containing 100 nM 7t15-21s and 50 nM of anti-TF antibody. As the volume of the cultures increased, the cells were transferred to higher volume flasks. The cells were counted using trypan blue to access the fold-expansion. 7t15-21s+anti-TF antibody-expanded NK cells were washed three times in warm HBSS Buffer (Hyclone) at 1000 RPM for 10 minutes at room temperature. The 7t15-21s+anti-TF antibody-expanded-NK cells were resuspended in 10×10⁶/0.2 mL HBSS buffer and injected intravenously into the tail vein of NSG mice (NOD scid common gamma mouse) (Jackson Laboratories). The transferred NK cells were supported every 48 hours with either 7t15-21s (10 ng/dose, i.p.), TGFRt15-TGFRs (10 ng/dose, i.p.) or 2t2 (10 ng/dose, i.p.) for up to 21 days. Engraftment and persistence of the human 7t15-21s+anti-TF antibody-expanded NK cells were measured every week in blood staining for hCD45, mCD45, hCD56, hCD3, and hCD16 antibodies by flow cytometry (Celesta-BD Bioscience) (Data represent 3 mice per group). FIG. 34 indicates that treatment of mice bearing adoptively-transferred 7t15-21s+anti-TF antibody-expanded NK cells with 7t15-21s-, TGFRt15-TGFRs-, or 2t2-induced expansion and persistence of the adoptively transferred NK cells compared to control treated mice.

### Example 7: NK-Mediated Cytotoxicity Following Treatment with Single-Chain Constructs or Multi-Chain Constructs

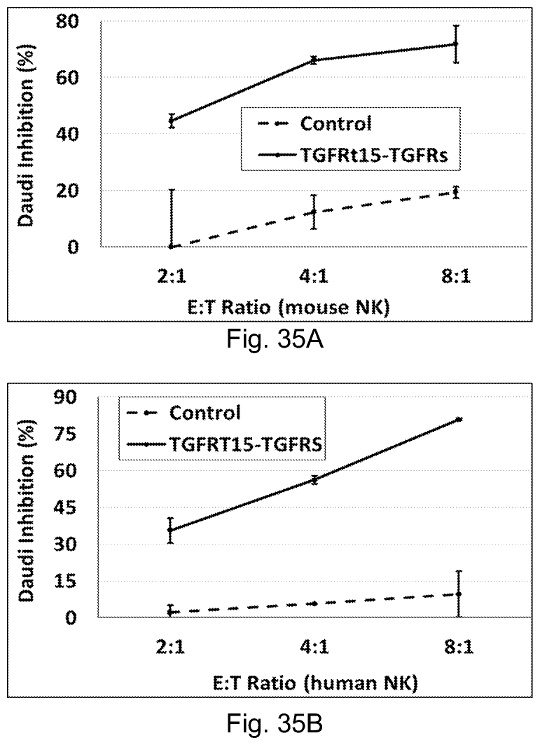

A set of experiments was performed to determine if treatment of NK cells with TGFRt15-TGFRs enhanced cytotoxicity of NK cells. In these experiments, Human Daudi B lymphoma cells were labeled with CellTrace Violet (CTV) and used as tumor target cells. Mouse NK effector cells were isolated with NK1.1-positive selection using a magnetic cell sorting method (Miltenyi Biotec) of C57BL/6 female mouse spleens 4 days post TGFRt15-TGFRs subcutaneous treatment at 3 mg/kg. Human NK effector cells were isolated from peripheral blood mononuclear cells derived from human blood buffy coats with the RosetteSep/human NK cell reagent (Stemcell Technologies). The target cells (Human Daudi B lymphoma cells) were mixed with effector cells (either mouse NK effector cells or human NK effector cells) in the presence of 50 nM TGFRt15-TGFRs or in the absence of TGFRt15-TGFRs (control) and incubated at 37° C. for 44 hours for mouse NK cells and for 20 hours for human NK cells. Target cell (Daudi) viability was assessed by analysis of propidium iodide-positive, CTV-labeled cells using flow cytometry. The percentage of Daudi inhibition was calculated using the formula (1−viable tumor cell number in experimental sample/viable tumor cell number in the sample without NK cells)×100. FIG. 35 shows that mouse (FIG. 35A) and human (FIG. 35B) NK cells had significantly stronger cytotoxicity against Daudi B cells following NK cell activation with TGFRt15-TGFRs than in the absence of TGFRt15-TGFRs activation.

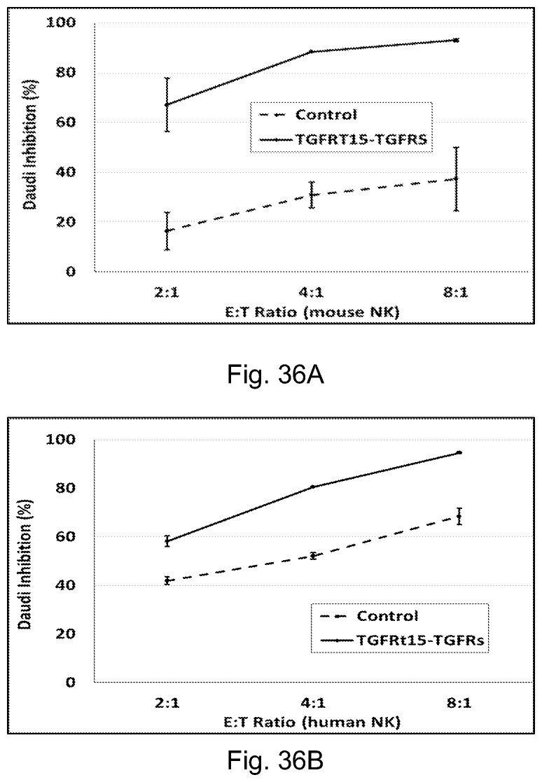

A set of experiments was performed to determine antibody-dependent cellular cytotoxicity (ADCC) of mouse and human NK cells following treatment with TGFRt15-TGFRs. In these experiments, human Daudi B lymphoma cells were labeled with CellTrace Violet (CTV) and used as tumor target cells. Mouse NK effector cells were isolated with NK1.1-positive selection using a magnetic cell sorting method (Miltenyi Biotec) of C57BL/6 female mouse spleens 4 days post-TGFRt15-TGFRs subcutaneous treatment at 3 mg/kg. Human NK effector cells were isolated from peripheral blood mononuclear cells derived from human blood buffy coats with the RosetteSep/human NK cell reagent (Stemcell Technologies). The target cells (Daudi B cells) were mixed with effector cells (either mouse NK effector cells or human NK effector cells) in the presence of anti-CD20 antibody (10 nM Rituximab, Genentech) and in the presence of 50 nM TGFRt15-TGFRs, or in the absence of TGFRt15-TGFRs (control) and incubated at 37° C. for 44 hours for mouse NK cells and for 20 hours for human NK cells. The Daudi B cells express the CD20 targets for the anti-CD20 antibody. Target cell viability was assessed after incubation by analysis of propidium iodide-positive, CTV-labeled target cells using flow cytometry. The percentage of Daudi inhibition was calculated using the formula (1−viable tumor cell number in experimental sample/viable tumor cell number in the sample without NK cells)×100. FIG. 36 shows that mouse NK cells (FIG. 36A) and human NK cells (FIG. 36B) had stronger ADCC activity against Daudi B cells following NK cell activation with TGFRt15-TGFRs than in the absence of TGFRt15-TGFRs activation.

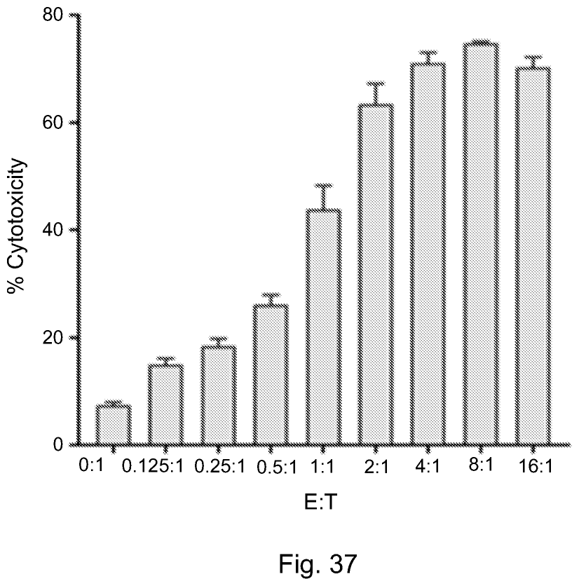

A set of experiments was performed to determine cytotoxicity of TGFRt15-TGFRs-activated mouse NK cells towards senescent B16F10 melanoma cells. In these experiments, mouse NK cells were activated in vivo by injecting C57BL/6 mice with 10 mg/kg of TGFRt15-TGFRs for 4 days followed by isolation of splenic NK cells. The NK cells were then expanded in vitro for 7 days in the presence of 100 nM 2t2. The B16F10 senescent target cells (B16F10-SNC) were labelled with CellTrace Violet (CTV) and incubated at different Effector:Target (E:T) ratios with the activated mouse NK effector cells for 16 hours. The cells were trypsinized, washed, and resuspended in complete media containing propidium iodide (PI) solution. The cytotoxicity of the TGFRt15-TGFRs/2t2-activated NK cells against the senescent cell targets was accessed by flow cytometry based on PI staining of the CTV-labeled cells. The findings demonstrate that in vivo activation of NK cells with TGFRt15-TGFRs followed by in vitro expansion and activation with 2t2 resulted in increased killing of senescent melanoma tumor cells by the NK cells (FIG. 37).

### Example 8: Treatment of Cancer, Diabetes, and Atherosclerosis

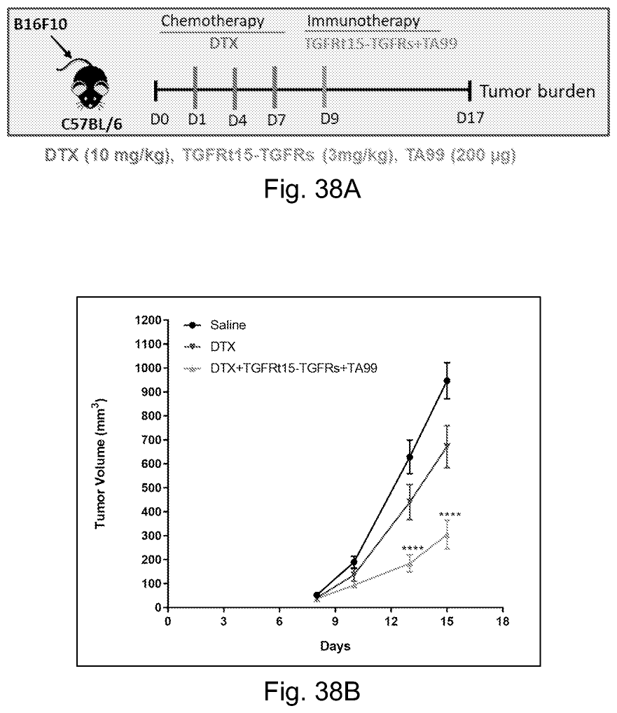

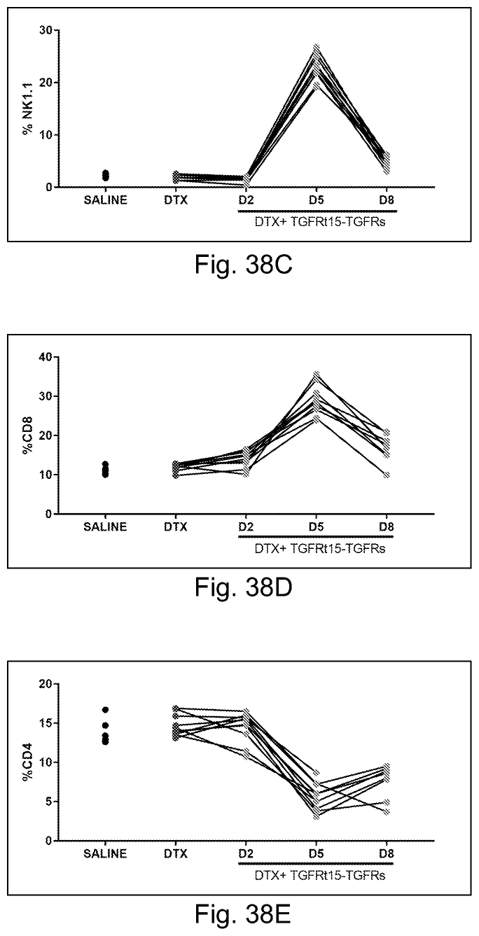

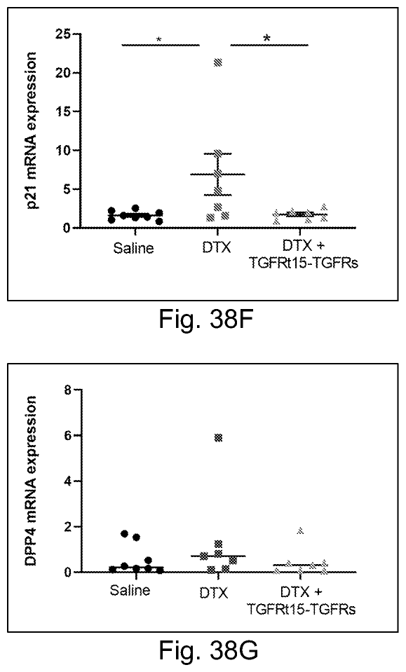

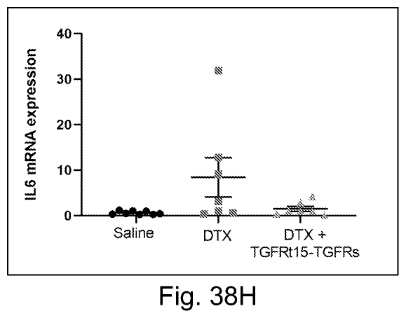

A set of experiments was performed to assess antitumor activity of TGFRt15-TGFRs plus anti-TRP1 antibody (TA99) in combination with chemotherapy in a melanoma mouse model. In these experiments, C57BL/6 mice were subcutaneously injected with 0.5×10⁶ B16F10 melanoma cells. The mice were treated with three doses of chemotherapy docetaxel (10 mg/kg) (DTX) on day 1, day 4, and day 7, followed by treatment with single dose of combination immunotherapy TGFRt15-TGFRs (3 mg/kg)+anti-TRP1 antibody TA99 (200 μg) on day 9. FIG. 38A shows a schematic of the treatment regimen. Tumor growth was monitored by caliper measurement, and tumor volume was calculated using the formula V=(L×W²)/2, where L is the largest tumor diameter and W is the perpendicular tumor diameter. FIG. 38B shows that treatment with DTX+TGFRt15-TGFRs+TA99 significantly reduced tumor growth compared to saline control and DTX treatment groups (N=10, ****p<0.001, Multiple t test analyses).

To assess immune cell subsets in the B16F10 tumor model, peripheral blood analysis was performed. In these experiments, C57BL/6 mice were injected with B16F10 cells and treated with DTX, DTX+TGFRt15-TGFRs+TA99, or saline. Blood was drawn from the submandibular vein of B16F10 tumor-bearing mice on days 2, 5, and 8 post-immunotherapy for the DTX+TGFRt15-TGFRs+TA99 group and day 11 post-tumor injection for the DTX and saline groups. RBCs were lysed in ACK lysis buffer and the lymphocytes were washed and stained with anti-NK1.1, anti-CD8, and anti-CD4 antibodies. The cells were analyzed by flow cytometry (Celesta-BD Bioscience). FIGS. 38C-38E show that DTX+TGFRt15-TGFRs+TA99 treatment induced an increase in the percentage of NK cells and CD8⁺ T cells in the tumors compared to the saline and DTX treatment groups.

On day 17, total RNA was extracted from tumors of mice treated with saline, DTX or DTX+TGFRt15-TGFRs+TA99 using Trizol. Total RNA (1 μg) was used for cDNA synthesis using the QuantiTect Reverse Transcription Kit (Qiagen). Real-time PCR was carried out with CFX96 Detection System (Bio-Rad) using FAM-labeled predesigned primers for senescence cell markers, (F) p21 (G) DPP4 and (H) IL6. The housekeeping gene 18S ribosomal RNA was used as an internal control to normalize the variability in expression levels. The expression of each target mRNA relative to 18S rRNA was calculated based on Ct as 2^{-Δ(ΔCt)}, in which ΔCt=Ct_{target}−Ct_{18S}. The data is presented as fold-change as compared to saline control. FIG. 38F-38H show that DTX treatment induced an increase in senescent tumor cells that were subsequently reduced following treatment with TGFRt15-TGFRs+TA99 immunotherapy.

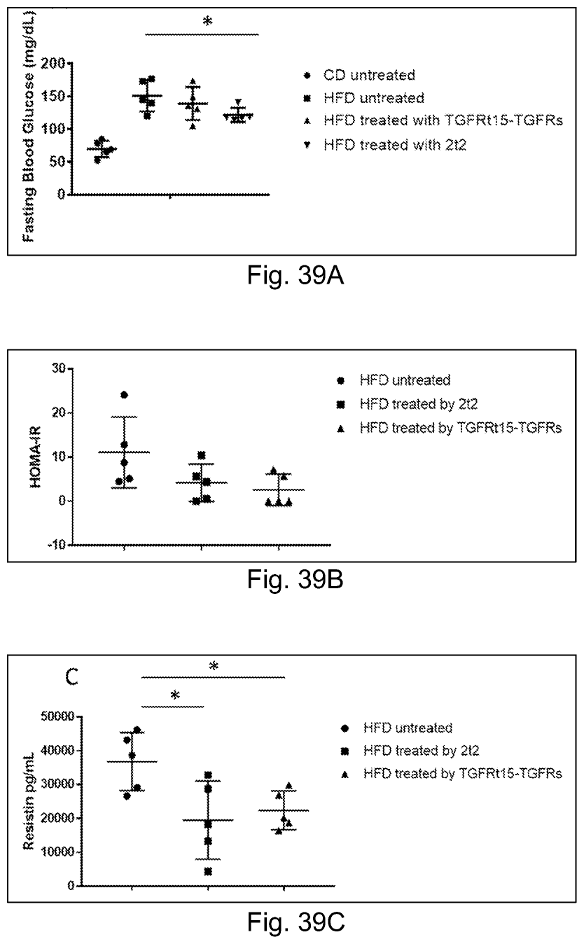

A set of experiments was performed to investigate amelioration of Western diet-induced hyperglycemia in ApoE^{-/-} mice by 2t2. In these experiments, 6-week old female B6.129P2-ApoE^{tm1Unc}/J mice (Jackson Laboratory) were fed with a Western diet containing 21% fat, 0.15% cholesterol, 34.1% sucrose, 19.5% casein, and 15% starch (TD88137, Envigo Laboratories). After 8-weeks of the Western diet, the mice were injected subcutaneously with TGFRt15-TGFRs or 2t2 at 3 mg/kg. Three days post-treatment, the mice were fasted for 16 hours and then blood samples were collected through retro-orbital venous plexus puncture. Blood glucose was detected with a glucose meter (OneTouch UltraMini) and GenUltimated test strips using a drop of fresh blood. As shown in FIG. 39A, 2t2 treatment significantly reduced hyperglycemia induced by the Western diet (p<0.04). The plasma insulin and resistin levels were analyzed with Mouse Rat Metabolic Array by Eve Technologies. HOMA-IR was calculated using the following formula: homeostatic model assessment-insulin resistance=Glucose (mg/dL)*Insulin (mU/mL)/405. As shown in FIG. 39B, both 2t2 and TGFRt15-TGFRs treatment reduced insulin resistance compared to the untreated group. Both 2t2 (p<0.02) and TGFRt15-TGFRs (p<0.05) reduced resistin levels significantly compared to the untreated group as shown in FIG. 39C, which may relate to the reduced insulin resistance induced by 2t2 and TGFRt15-TGFRs (FIG. 39B).

### Example 9: Upregulation of CD44 Memory T Cells

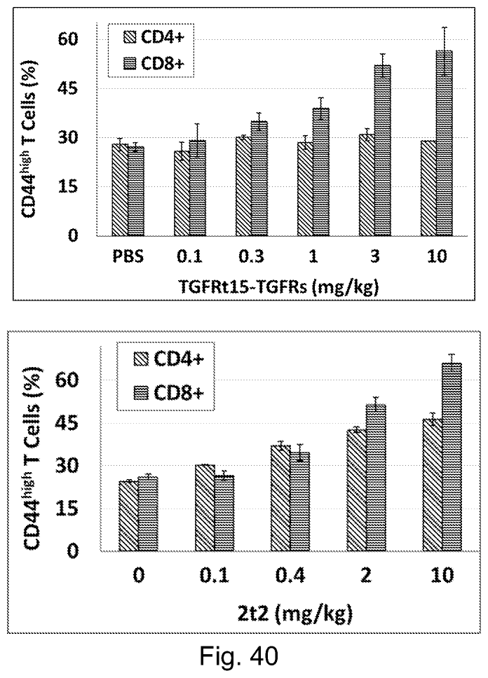

C57BL/6 mice were subcutaneously treated with TGFRt15-TGFRs or 2t2. The treated mice were euthanized and the single splenocyte suspensions were prepared 4 days (TGFRt15-TGFRs) or 3 days (2t2) following the treatment. The prepared splenocytes were stained with fluorochrome-conjugated anti-CD4, anti-CD8 and anti-CD44 antibodies and the percentages of CD44^{high} T cells in CD4⁻ T cells or CD8⁺ T cells were analyzed by flow cytometry. The results show that TGFRt15-TGFRs and 2t2 upregulated expression of the memory marker CD44 on CD4⁺ and CD8⁺ T cells (FIG. 40). These findings indicate that TGFRt15-TGFRs and 2t2 molecules were able to induce mouse T cells to differentiate into memory T cells.

### Example 10: Immuno-Phenotype and Cell Proliferation Following Treatment with IL-15-Based Agents (Day 3 Post Treatment)

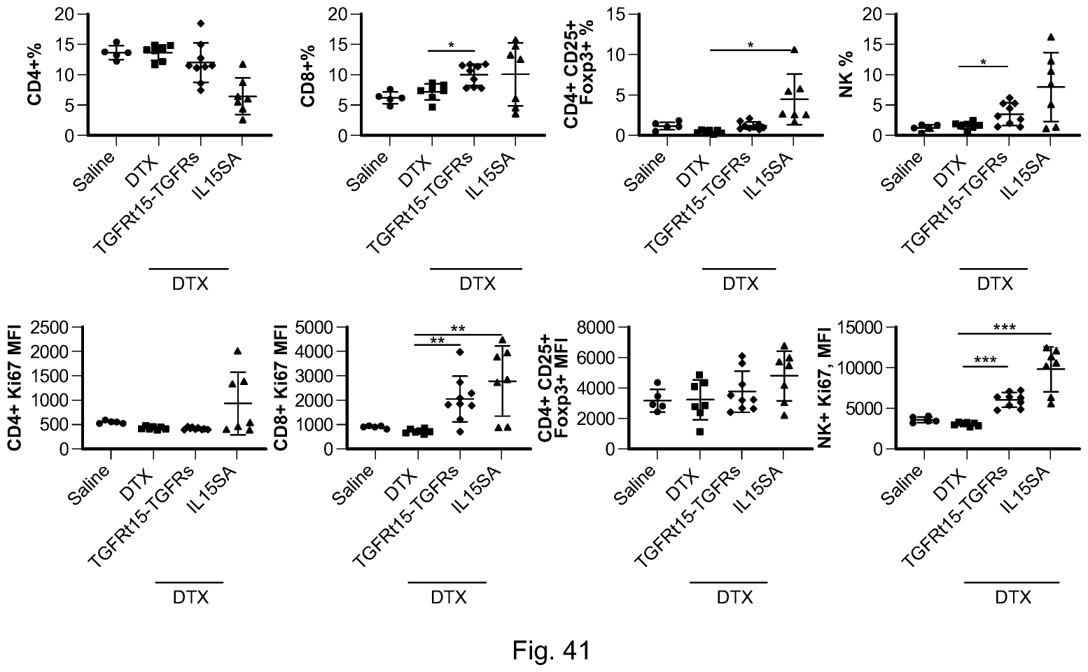

The mouse blood was prepared in order to evaluate the different subsets of immune cells after treatment with TGFRt15-TGFRs. C57BL/6, 6-week-old mice were purchased from The Jackson Laboratory. Mice were housed in a temperature and light controlled environment. Mice were divided into groups as follows: Saline control group (n=6), docetaxel group (n=6), docetaxel with TGFRt15-TGFRs group (n=6) and docetaxel with IL-15SA group (n=6). The IL-15 superagonist (IL-15SA) was constructed and administered as previously described (Zhu et al., J. Immunol. 183 (6): 3598-3607, 2009). Senescence was induced in mice with three doses of docetaxel (10 mg/kg) at day 1, 4 and 7. On day 8, mice were treated subcutaneously with either PBS or with TGFRt15-TGFRs (3 mg/kg) or with IL-15SA (0.2 mg/kg). The mouse blood was collected from submandibular vein on Day 3 post treatment in EDTA contained tubes. The whole blood was centrifuged to collect plasma @ 3000 RPM for 10 minutes in a micro centrifuge. Plasma was stored at −80° C. and whole blood was processed for immune cells phenotyping by flow cytometry. Whole bloods were lysed in ACK buffer for 5 minutes at room temperature. Cell were washed in FACS buffer (1×PBS (Hyclone) with 0.5% BSA (EMD Millipore) and 0.001% Sodium Azide (Sigma)). To assess the different types of immune cells in blood, cells were stained for cell-surface CD4, CD45, CD8 and NK1.1 (BioLegend) for 30 minutes at RT. After surface staining, cells were washed (1500 RPM for 5 minutes at room temperature) in FACS buffer (1×PBS (Hyclone) with 0.5% BSA (EMD Millipore) and 0.001% Sodium Azide (Sigma)). Cells were treated with permeabilization buffer (Invitrogen) for 20 min at 4° C. followed by wash with Perm buffer (Invitrogen). Cells were then stained for intracellular markers (Ki67) and FoxP3 for 30 min at room temperature. After two washes, cells were resuspended in fixation buffer and analyzed by Flow Cytometry (Celesta-BD Bioscience). These data show that IL-15-based agents TGFRt15-TGFRs and IL-15SA can stimulate and promote the expansion and proliferation of NK and CD8⁺ T cells after docetaxel treatment (FIG. 41).

### Example 11: TGFRt15-TGFRs Treatment Reduces Senescence-Associated Gene Expression in C57BL/6 Mice

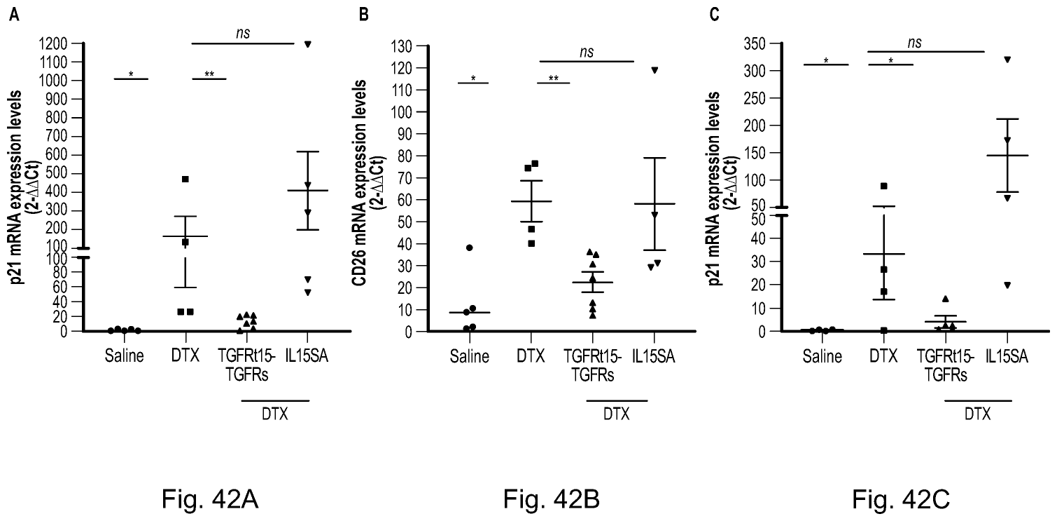

Chemotherapy induced senescence-associated gene expression was significantly reduced with TGFRt15-TGFRs in the lung and liver of C57BL/6 mice. C57BL/6 mice were treated with three doses of chemotherapy docetaxel (10 mg/kg) at day 1, day 4 and day 7. On day 8, docetaxel treated mice were divided into three groups. The first group received no treatment, second group received TGFRt15-TGFRs and third group received IL-15SA. Saline treated mice were used as controls. The TGFRt15-TGFRs was administered at a dosage of 3 mg/kg and IL-15SA was administered at 0.2 mg/kg. On Day 3 post-study drug treatment, the mice were sacrificed and lung and liver were collected. FIGS. 42A-42C show expression of p21^{CIP1}p21 and CD26 in lung (FIGS. 42A and 42B) and p21^{CIP1}p21 in liver (FIG. 42C) tissues respectively. Lung and liver tissues were homogenized by using mortar and pestle in liquid nitrogen. Homogenized tissues were transferred in fresh Eppendorf tubes containing 1 mL of Trizol (Thermo Fischer). Total RNA was extracted using RNeasy Mini Kit (Qiagen #74106) according to the manufacturer's instructions. 1 μg of total RNA was used for cDNA synthesis using the QuantiTect Reverse Transcription Kit (Qiagen). Real-time PCR was carried out with CFX96 Detection System (Bio-Rad) using FAM labeled predesigned primers p21^{CIP1}p21 and CD26 were purchased from Thermo Scientific. Reactions were run in triplicate for all the genes examined. The housekeeping gene 18S ribosomal RNA was used as an internal control to normalize the variability in expression levels. The expression of each target mRNA relative to 18S rRNA was calculated based on Ct as 2^{-Δ(ΔCt)}, in which ΔCt=Ct_{target}−Ct_{18S}.

As shown in FIGS. 42A-42C, the therapy-induced senescence marker p21^{CIP1}p21 was significantly reduced in the lung and liver tissues of mice treated with TGFRt15-TGFRs. The therapy-induced senescence marker CD26 was also significantly reduced in the lung tissues of mice treated with TGFRt15-TGFRs.

### Example 12: Immuno-Phenotype Following Treatment with IL-15-Based Agents

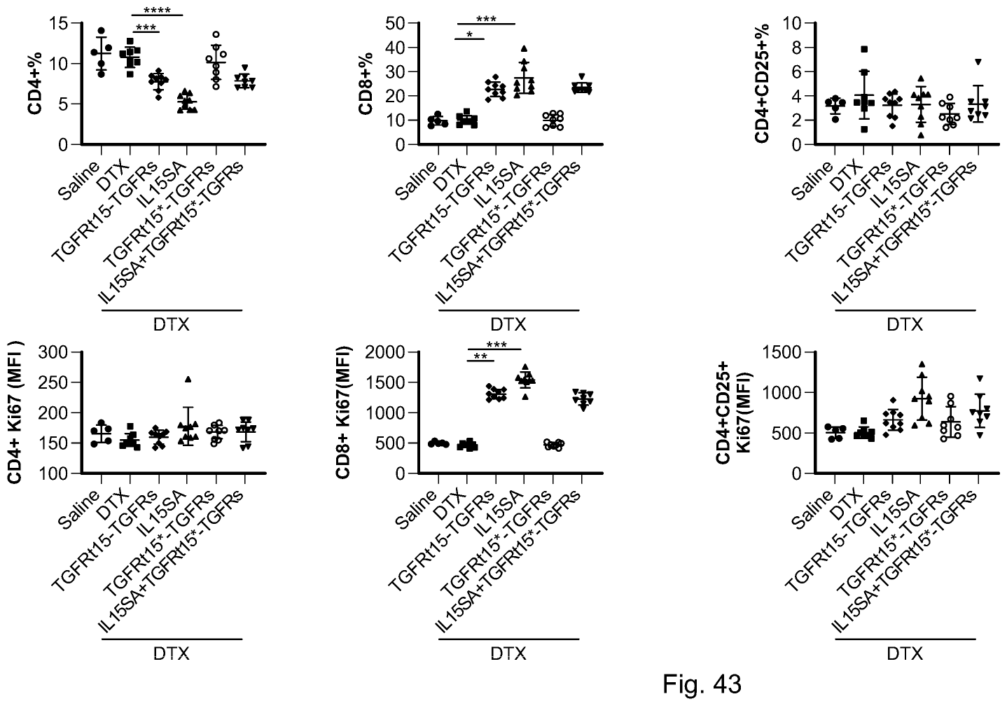

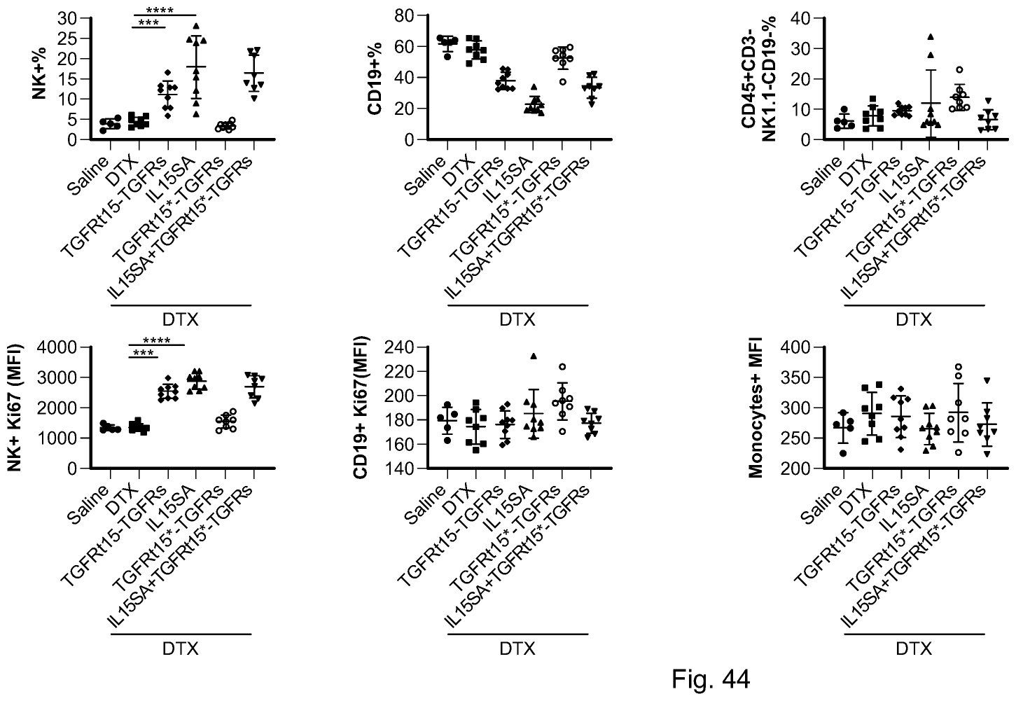

The mouse blood was prepared in order to evaluate the different subsets of immune cells after treatment with IL-15-based agents: TGFRt15-TGFRs, an IL-15 superagonist (IL-15SA) and an IL-15 fusion with a D8N mutant knocking out the IL-15 activity (TGFRt15*-TGFRs). C57BL/6, 6-week-old mice were purchased from The Jackson Laboratory. Mice were housed in a temperature and light controlled environment. Mice were divided into groups (n=6/group) and treated with the following: 1) PBS (saline) control, 2) docetaxel, 3) docetaxel with TGFRt15-TGFRs, 4) docetaxel with IL-15SA, 5) docetaxel with an IL-15 mutant (TGFRt15*-TGFRs) and 6) docetaxel with an IL-15 superagonist (IL-15SA) plus TGFRt15*-TGFRs. Senescence was induced in mice with three dose of docetaxel (10 mg/kg) at day 1, 4 and 7. On day 8, the mice were treated subcutaneously with PBS, TGFRt15-TGFRs, TGFRt15*-TGFRs, IL-15SA or in combinations as discussed above. TGFRt15-TGFRs and TGFRt15*-TGFRs were administered at a dosage of 3 mg/kg and IL-15SA was administered at 0.05 mg/kg. The mouse blood was collected from the submandibular vein on day 3 post-study drug treatment into EDTA tubes. The whole blood was centrifuged to collect plasma at 3000 RPM for 10 minutes in a micro centrifuge. Plasma was stored at −80° C. and whole blood was processed for immune cell phenotyping by flow cytometry. Whole blood was lysed in ACK buffer for 5 minutes at 37° C. Cell were washed in FACS buffer (1×PBS (Hyclone) with 0.5% BSA (EMD Millipore) and 0.001% Sodium Azide (Sigma)). To assess the different types of immune cells in the blood, cells were stained for cell-surface CD4, CD45, CD19 CD8 and NK1.1 (BioLegend) for 30 minutes at room temperature (RT). After surface staining, cells were washed (1500 RPM for 5 minutes at room temperature) in FACS buffer (1×PBS (Hyclone) with 0.5% BSA (EMD Millipore) and 0.001% Sodium Azide (Sigma)). Cells were treated with permeabilization buffer (Invitrogen) for 20 min at 4° C. followed by wash with Perm buffer (Invitrogen). Cells were then stained for intracellular markers (Ki67) for 30 min at RT. After two washes, cells were resuspended in fixation buffer and analyzed by Flow Cytometry (Celesta-BD Bioscience) (FIG. 43 and FIG. 44).

These data show that IL-15-based agents TGFRt15-TGFRs and IL-15SA can stimulate and promote the expansion and proliferation of NK and CD8⁺ T cells after docetaxel treatment. Increased NK and CD8⁺ T cell expansion and proliferation was not seen with fusion proteins lacking IL-15 activity (i.e., TGFRt15*-TGFRs).

### Example 13: Evaluation of Senescence Markers p21^{CIP1}p21 and CD26 in Lung and Liver Tissues

Markers for cellular senescence were evaluated in tissues of normal mice following chemotherapy and administration of study treatments. C57BL/6, 6-week-old mice were purchased from The Jackson Laboratory. Mice were housed in a temperature and light controlled environment. Mice were divided into six groups and treated with the following: 1) PBS (saline) control (n=5), 2) docetaxel (n=8), 3) docetaxel with TGFRt15-TGFRs (n=8), 4) docetaxel with IL15SA (n=8), 5) docetaxel with an IL-15 mutant (TGFRt15*-TGFRs) (n=8) and 6) docetaxel with an IL-15 superagonist (IL-15SA) plus TGFRt15*-TGFRs (n=6). Senescence was induced in mice with three doses of docetaxel (10 mg/kg) at day 1, 4 and 7. On day 8, the mice were treated subcutaneously with PBS, TGFRt15-TGFRs, TGFRt15*-TGFRs, IL-15SA or in combinations as discussed below. TGFRt15-TGFRs and TGFRt15*-TGFRs were administered at a dosage of 3 mg/kg and IL-15SA was administered at 0.05 mg/kg. The mouse tissues were prepared in order to evaluate the different senescence markers. Mice were euthanized on day 7 post-study drug treatment and the liver and lung tissues were harvested and stored in liquid nitrogen in 1.7 mL Eppendorf tubes. Samples were homogenized by using mortar and pestle in liquid nitrogen. Homogenized tissues were transferred in fresh Eppendorf tubes containing 1 mL of Trizol (Thermo Fischer). Total RNA was extracted using RNeasy Mini Kit (Qiagen #74106) according to the manufacturer's instructions and 1 μg of total RNA was used for cDNA synthesis using the QuantiTect Reverse Transcription Kit (Qiagen). Real-time PCR was carried out with CFX96 Detection System (Bio-Rad) using FAM labeled predesigned primers purchased from Thermo Scientific. Reactions were run in triplicate for all the genes examined. The housekeeping gene 18S ribosomal RNA was used as an internal control to normalize the variability in expression levels. The expression of each target mRNA relative to 18S rRNA was calculated based on Ct as 2^{-Δ(Δct)}, in which ΔCt=Ct_{target}−Ct_{18S}.

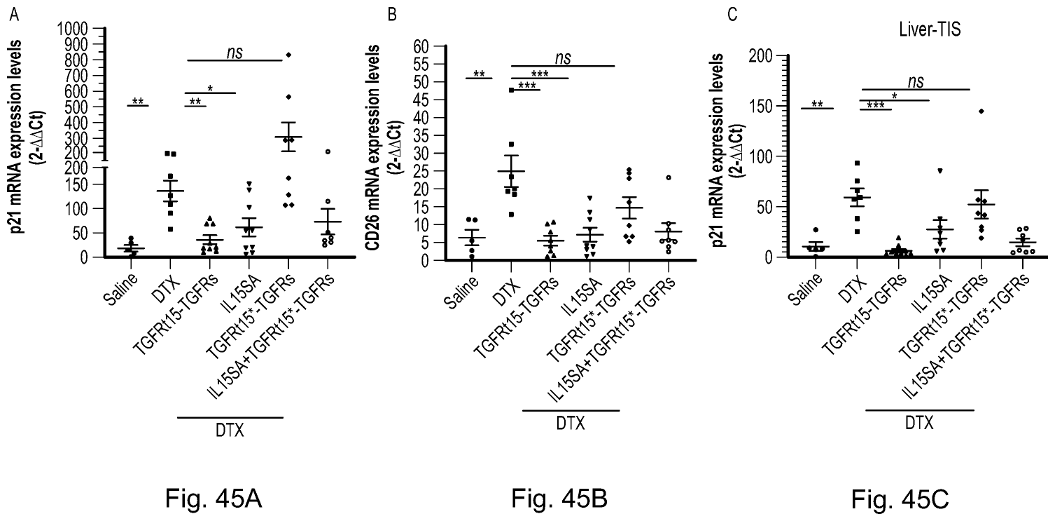

As shown in FIGS. 45A-45C, the senescence markers p21 and CD26 were induced in the lung (FIG. 45A and FIG. 45B, respectively) and p21^{CIP1}p21 in liver (FIG. 45C) tissues of mice treated with docetaxel. The senescence markers p21^{CIP1}p21 and CD26 in the lungs and p21^{CIP1}p21 in the liver were reduced of the mice treated with TGFRt15-TGFRs, IL-15SA and combination of IL-15SA and TGFRt15*-TGFRs mutant. However, the TGFRt15*-TGFRs mutant treated mice lung failed to eliminate the senescence markers in these tissues. These results show that IL-15 activity is important for clearance of TIS senescence cells.

### Example 14: Immuno-Phenotype Following Treatment with TGFRt15-TGFRs

The mouse blood was prepared in order to evaluate the different subsets of immune cells after treatment with TGFRt15-TGFRs. C57BL/6, 76-week-old aged mice were purchased from The Jackson Laboratory. Mice were housed in a temperature and light controlled environment. Mice were divided into two groups as follows: PBS control group (n=6) and TGFRt15-TGFRs group (n=6). Mice were treated subcutaneously with either PBS or with TGFRt15-TGFRs at a dosage of 3 mg/kg on Day 0. On Day 4 following the first dose of study treatment, the mouse blood was collected from the submandibular vein in EDTA contained tubes. The whole blood was centrifuged to collect plasma at 3000 RPM for 10 minutes in a micro centrifuge. Plasma was stored at −80° C. and the blood was processed for immune cell phenotyping by flow cytometry. Whole blood was lysed in ACK buffer for 5 minutes at room temperature. Cells were washed in FACS buffer (1×PBS (Hyclone) with 0.5% BSA (EMD Millipore) and 0.001% Sodium Azide (Sigma)). To assess the different types of immune cells in blood, cells were stained for cell-surface CD4, CD45, CD19 CD8 and NK1.1 (BioLegend) for 30 minutes at room temperature (RT). After surface staining, cells were washed (1500 RPM for 5 minutes at RT) in FACS buffer (1×PBS (Hyclone) with 0.5% BSA (EMD Millipore) and 0.001% Sodium Azide (Sigma)). Cells were treated with permeabilization buffer (Invitrogen) for 20 min at 4° C. followed by wash with Perm buffer (Invitrogen). Cells were then stained for intracellular markers (Ki67) for 30 min at RT. After two washes, cells were resuspended in fixation buffer and analyzed by flow cytometry (Celesta-BD Bioscience).

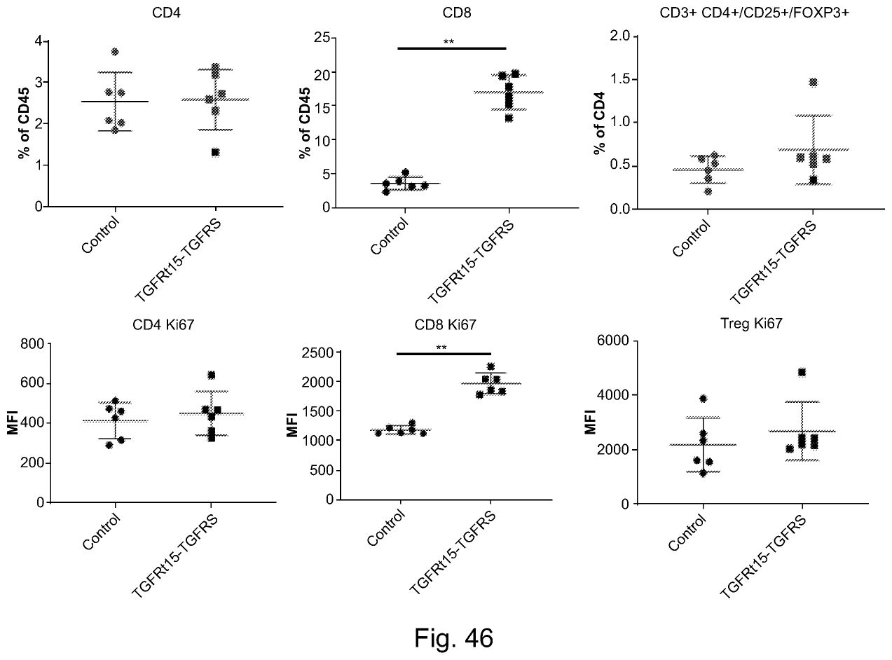

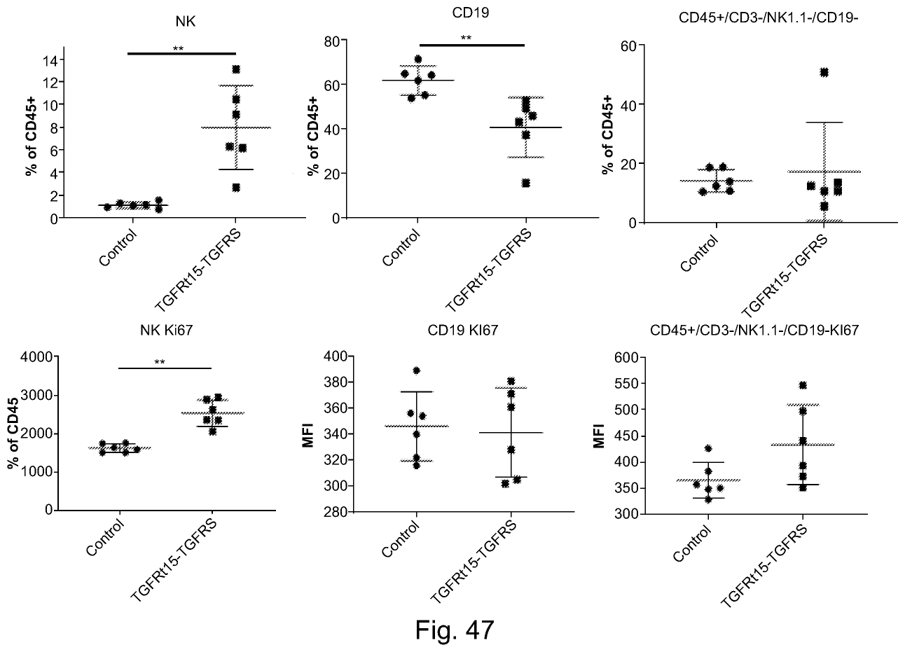

As shown in FIG. 46, the percentages of CD8⁺ T cells and proliferation of CD8⁺ T cells, which was measured by Ki67, significantly increased, 4 days after the first dose of TGFRt15-TGFRs. We also observed an increase in NK cells and proliferation of NK cells as shown in FIG. 47. We observed significant decreases in CD19⁺ cells after the first dose of TGFRt15-TGFRs. These results demonstrate that a single dose of TGFRt15-TGFRs administered subcutaneously can stimulate immune cells, such as CD8⁺ T cells and NK cells to proliferate in the blood of aged mice.

### Example 15: TGFRt15-TGFRs Reduces Senescence-Associated β-Gal from Liver and Lung Tissues

The mouse liver and lungs were prepared in order to evaluate the senescence-associated β-gal in tissues after treatment with TGFRt15-TGFRs. C57BL/6, 76-week-old aged mice were purchased from The Jackson Laboratory. Mice were housed in a temperature and light controlled environment. Mice were divided into two groups as follows: PBS control group (n=6) and TGFRt15-TGFRs group (n=6). Mice were treated subcutaneously with either PBS or with TGFRt15-TGFRs at a dosage of 3 mg/kg on Day 0 and Day 10. On Day 7 following the second dose of study treatment, mice were euthanized and liver and lungs were harvested, homogenized in PBS containing 2% PBS, and filtered in 70-micron filter to obtain a single cell suspension. Cells were spun down then resuspended in 5 mL RPMI containing 0.5 mg/mL collagenase IV and 0.02 mg/mL DNAse in 14 mL round bottom tubes. Then, the cells were shaken on orbital shaker for 1 hr at 37° C. The cells were washed twice with RPMI. Cells were resuspended at 2×10⁶/mL in a 24 well flat bottom plate in 2 mL of complete media (RPMI 1640 (Gibco) supplemented with 2 mM L-glutamine (Thermo Life Technologies), penicillin (Thermo Life Technologies), streptomycin (Thermo Life Technologies), and 10% FBS (Hyclone)) and cultured for 48 hrs at 37° C., 5% CO₂. Cells were harvested, washed once in warm complete media at 1000 rpm for 10 minutes at room temperature. The cell pellet was resuspended in 500 μL of fresh media containing 1.5 μL of Senescence Dye per tube. Then, the cells were further incubated for 1-2 hr at 37° C., 5% CO₂ and washed 2× with 500 μL Wash buffer. Cell pellet was resuspended cells in 500 μL of wash buffer and was analyzed immediately by flow cytometry (Celesta-BD Bioscience).

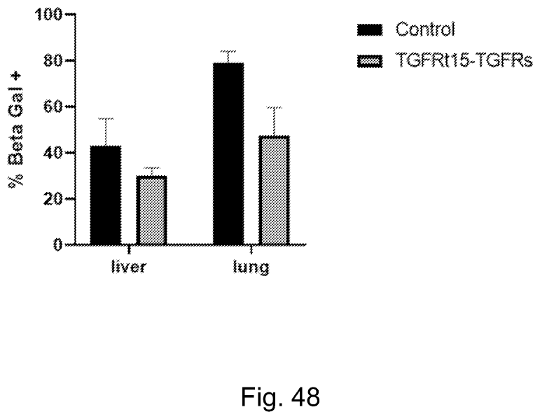

As shown in FIG. 48, the percentages of senescence-associated β-gal⁺ cells decreased 7 days following the second dose of TGFRt15-TGFRs. These results demonstrate that TGFRt15-TGFRs can reduce the senescence-associated β-gal in tissues of aged mice.

### Example 16: Senescence Markers CD26, IL-1α, p16INK4 and p21^{CIP1} in Kidney, Skin, Liver and Lung Tissues

The mouse kidney, skin, liver and lungs were harvested in order to evaluate the senescence markers CD26, IL-1α, p16 and p21 by quantitative PCR in tissues after treatment with TGFRt15-TGFRs or the PBS control group. C57BL/6, 76-week-old aged mice were purchased from The Jackson Laboratory. Mice were housed in a temperature and light controlled environment for one week before performing any study. Mice were divided into two groups as follows: PBS control group (n=6) and TGFRt15-TGFRs group (n=6). Mice were treated subcutaneously either with PBS or with TGFRt15-TGFRs at a dosage of 3 mg/kg on Day 0 and Day 10. On Day 7 following the second dose of study treatment, mice were euthanized and the kidney, skin, liver and lung were harvested and stored in liquid nitrogen in 1.7 mL Eppendorf tubes. Samples were homogenized by using mortar and pestle in liquid nitrogen. Homogenized tissues were transferred in fresh Eppendorf tubes containing 1 mL of Trizol (Thermo Fischer). Total RNA was extracted using RNeasy Mini Kit (Qiagen #74106) according to the manufacturer's instructions and 1 μg of total RNA was used for cDNA synthesis using the QuantiTect Reverse Transcription Kit (Qiagen). Real-time PCR was carried out with CFX96 Detection System (Bio-Rad) using FAM labeled predesigned primers purchased from Thermo Scientific. Reactions were run in triplicate for all the genes examined. The housekeeping gene 18S ribosomal RNA was used as an internal control to normalize the variability in expression levels. The expression of each target mRNA relative to 18S rRNA was calculated based on Ct as 2^{-Δ(Δct)}, in which ΔCt=Ct_{target}−Ct_{18S}.

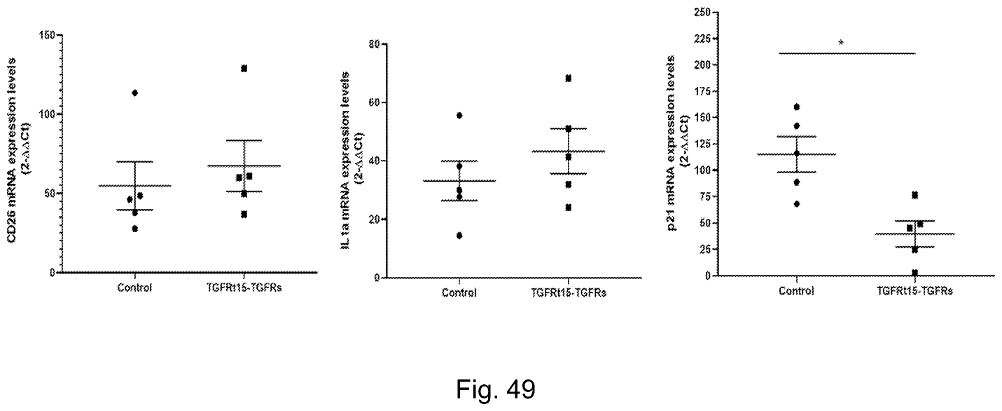

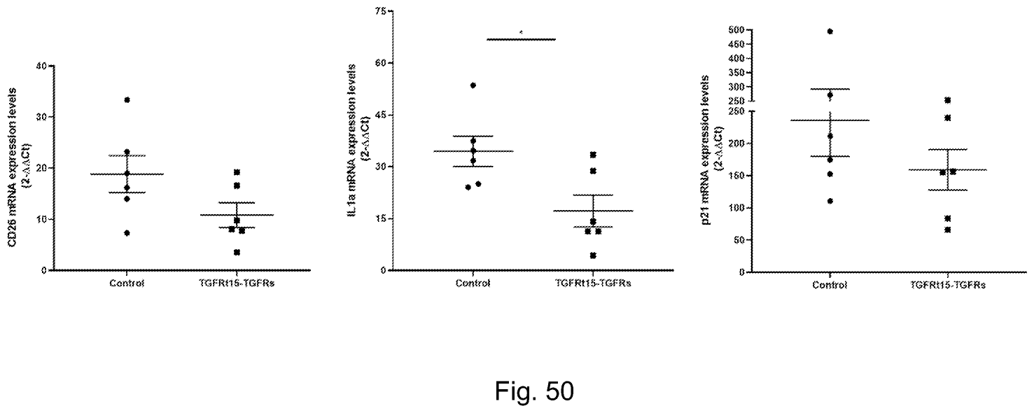

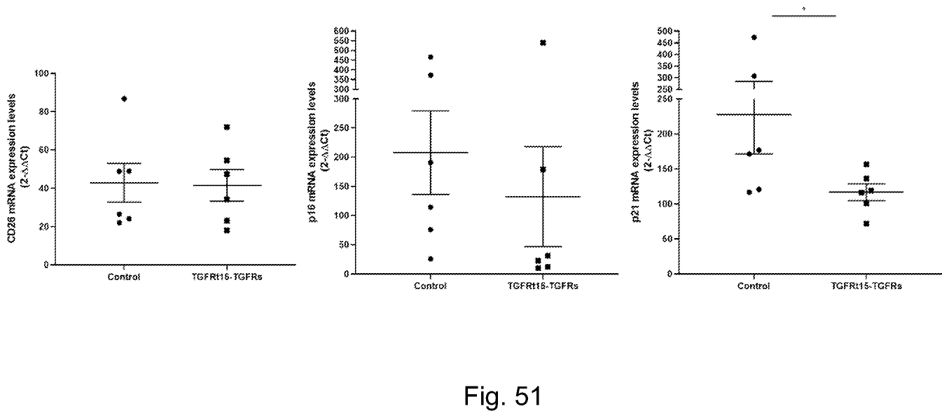

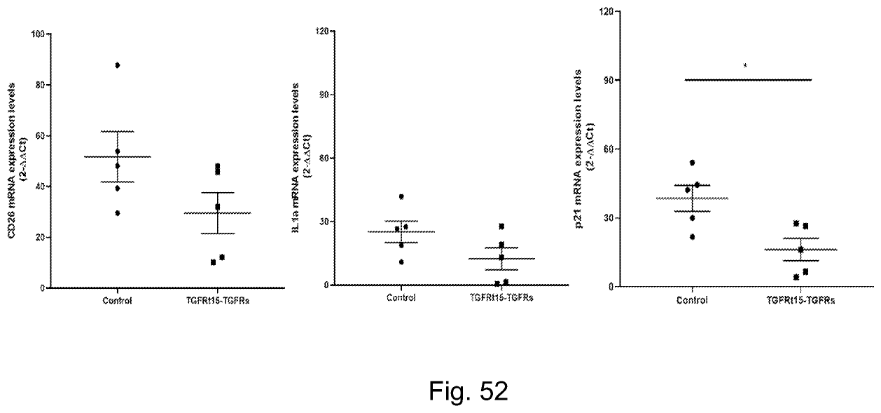

As shown in FIGS. 49-52, there was no difference in senescence markers CD26 and IL-1α, however p21^{CIP1} showed decreased expression in the liver (FIG. 49), lung (FIG. 52) and skin (FIG. 51) of TGFRt15-TGFRs-treated-mice. In the kidney (FIG. 50), both p21^{CIP1} and IL1α markers were significantly decreased in the aged mice 7 days after the second dose of TGFRt15-TGFRs.

### Example 17: β-Gal Staining on Kidney Tissues by Histology

The mouse kidney was prepared in order to evaluate senescence marker β-gal in kidney tissues after treatment with TGFRt15-TGFRs. C57BL/6, 76-week-old aged mice were purchased from The Jackson Laboratory. Mice were housed in a temperature and light controlled environment. Mice were divided into two groups as follows: PBS control group (n=6) and TGFRt15-TGFRs group (n=6). Mice were treated subcutaneously with either PBS or with TGFRt15-TGFRs at a dosage of 3 mg/kg on Day 0 and Day 10. On Day 7 following the second dose of study treatment, mice were euthanized and the kidneys were harvested, and half of the kidney tissue was embedded in tissue-tek cyromolds contain OCT compound. Tissue-tek cyromolds containing tissue were immediately frozen down in the vapor phase of liquid nitrogen. Samples were further processed to cut 4-8 um thick cryostat sections (Lecia Cm 1800 Cryostat) and mounted on superfrost plus slides. Slides with sections were processed for senescence b-galactosidase staining kit (Cell Signaling) as per manufacturer's protocol. Tissue sections were observed under microscope.

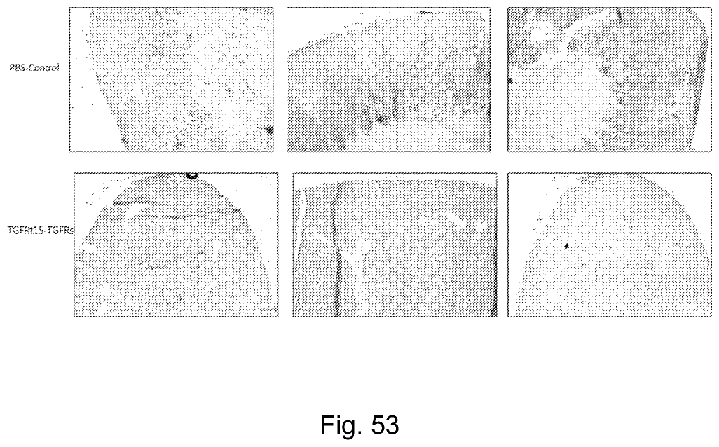

As shown in FIG. 53, decreased numbers of senescence-associated β-gal⁺ cells were observed in TGFRt15-TGFRs treated mice compared to control mice (n=3). These results demonstrate that TGFRt15-TGFRs treatment is able to reduce senescence-associated β-gal in tissues of aged mice.

### Example 18: TGFRt15*-TGFRs Fusion Protein Generation

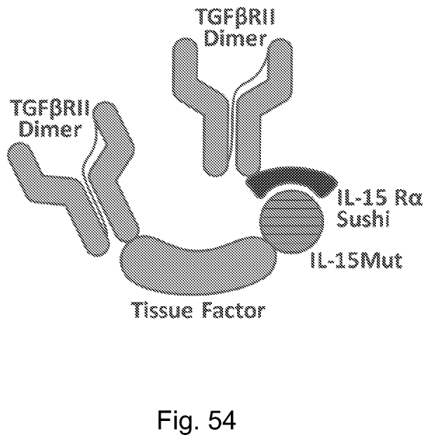

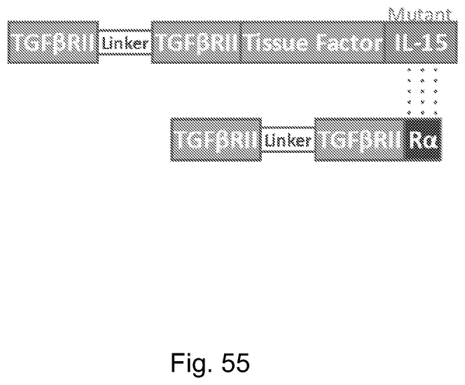

A fusion protein complex was generated comprising of TGFR/IL15RαSu and TGFR/TF/IL-15D8N fusion proteins (FIGS. 54 and 55). The human TGF-β receptor (TGFR), IL-15 alpha receptor sushi domain (IL15RαSu), tissue factor (TF) and IL-15 with D8N mutant (IL15D8N) sequences were obtained from the GenBank website and DNA fragments for these sequences were synthesized by Genewiz. Specifically, a construct was made linking the TGFR sequence to the N-terminus coding region of IL15RαSu and the TGFR sequence to the N-terminus of tissue factor 219 followed by the N-terminus coding region of IL-15D8N.

The nucleic acid sequence of the TGFR/IL15RaSu_construct (including signal peptide sequence) is as follows:

(Signal peptide)
ATGAAGTGGGTGACCTTCATCAGCCTGCTGTTCCTGTTCTCCAGCGCCT
ACTCC
(Single chain Human TGF-beta Receptor II
homodimer)
ATCCCCCCCCATGTGCAAAAGAGCGTGAACAACGATATGATCGTGACC
GACAACAACGGCGCCGTGAAGTTTCCCCAGCTCTGCAAGTTCTGCGATG
TCAGGTTCAGCACCTGCGATAATCAGAAGTCCTGCATGTCCAACTGCAG
CATCACCTCCATCTGCGAGAAGCCCCAAGAAGTGTGCGTGGCCGTGTGG
CGGAAAAATGACGAGAACATCACCCTGGAGACCGTGTGTCACGACCCCA
AGCTCCCTTATCACGACTTCATTCTGGAGGACGCTGCCTCCCCCAAATG
CATCATGAAGGAGAAGAAGAAGCCCGGAGAGACCTTCTTTATGTGTTCC
TGTAGCAGCGACGAGTGTAACGACAACATCATCTTCAGCGAAGAGTACA
ACACCAGCAACCCTGATGGAGGTGGCGGATCCGGAGGTGGAGGTTCTGG
TGGAGGTGGGAGTATTCCTCCCCACGTGCAGAAGAGCGTGAATAATGAC
ATGATCGTGACCGATAACAATGGCGCCGTGAAATTTCCCCAGCTGTGCA
AATTCTGCGATGTGAGGTTTTCCACCTGCGACAACCAGAAGTCCTGTAT
GAGCAACTGCTCCATCACCTCCATCTGTGAGAAGCCTCAGGAGGTGTGC
GTGGCTGTCTGGCGGAAGAATGACGAGAATATCACCCTGGAAACCGTCT
GCCACGATCCCAAGCTGCCCTACCACGATTTCATCCTGGAAGACGCCGC
CAGCCCTAAGTGCATCATGAAAGAGAAAAAGAAGCCTGGCGAGACCTTT
TTCATGTGCTCCTGCAGCAGCGACGAATGCAACGACAATATCATCTTTA
GCGAGGAATACAATACCAGCAACCCCGAC
(Sushi domain of IL15 receptor alpha chain)
ATTACATGCCCCCCTCCCATGAGCGTGGAGCACGCCGACATCTGGGTG
AAGAGCTATAGCCTCTACAGCCGGGAGAGGTATATCTGTAACAGCGGCT
TCAAGAGGAAGGCCGGCACCAGCAGCCTCACCGAGTGCGTGCTGAATAA
GGCTACCAACGTGGCTCACTGGACAACACCCTCTTTAAAGTGCATCCGG

The nucleic acid sequence of the TGFR/TF/IL15D8N construct (including signal peptide sequence) is as follows:

(Signal peptide)
ATGGGAGTGAAAGTTCTTTTTGCCCTTATTTGTATTGCTGTGGCCGAGG
CC
(Single chain Human TGF-beta Receptor II
homodimer)
ATCCCACCGCACGTTCAGAAGTCGGTGAATAACGACATGATAGTCACT
GACAACAACGGTGCAGTCAAGTTTCCACAACTGTGTAAATTTTGTGATG
TGAGATTTTCCACCTGTGACAACCAGAAATCCTGCATGAGCAACTGCAG
CATCACCTCCATCTGTGAGAAGCCACAGGAAGTCTGTGTGGCTGTATGG
AGAAAGAATGACGAGAACATAACACTAGAGACAGTTTGCCATGACCCCA
AGCTCCCCTACCATGACTTTATTCTGGAAGATGCTGCTTCTCCAAAGTG
CATTATGAAGGAAAAAAAAAAGCCTGGTGAGACTTTCTTCATGTGTTCC
TGTAGCTCTGATGAGTGCAATGACAACATCATCTTCTCAGAAGAATATA
ACACCAGCAATCCTGACGGAGGTGGCGGATCCGGAGGTGGAGGTTCTGG
TGGAGGTGGGAGTATTCCTCCCCACGTGCAGAAGAGCGTGAATAATGAC
ATGATCGTGACCGATAACAATGGCGCCGTGAAATTTCCCCAGCTGTGCA
AATTCTGCGATGTGAGGTTTTCCACCTGCGACAACCAGAAGTCCTGTAT
GAGCAACTGCTCCATCACCTCCATCTGTGAGAAGCCTCAGGAGGTGTGC
GTGGCTGTCTGGCGGAAGAATGACGAGAATATCACCCTGGAAACCGTCT
GCCACGATCCCAAGCTGCCCTACCACGATTTCATCCTGGAAGACGCCGC
CAGCCCTAAGTGCATCATGAAAGAGAAAAAGAAGCCTGGCGAGACCTTT
TTCATGTGCTCCTGCAGCAGCGACGAATGCAACGACAATATCATCTTTA
GCGAGGAATACAATACCAGCAACCCCGAC
(Human Tissue Factor 219)
TCAGGCACTACAAATACTGTGGCAGCATATAATTTAACTTGGAAATCA
ACTAATTTCAAGACAATTTTGGAGTGGGAACCCAAACCCGTCAATCAAG
TCTACACTGTTCAAATAAGCACTAAGTCAGGAGATTGGAAAAGCAAATG
CTTTTACACAACAGACACAGAGTGTGACCTCACCGACGAGATTGTGAAG
GATGTGAAGCAGACGTACTTGGCACGGGTCTTCTCCTACCCGGCAGGGA
ATGTGGAGAGCACCGGTTCTGCTGGGGAGCCTCTGTATGAGAACTCCCC
AGAGTTCACACCTTACCTGGAGACAAACCTCGGACAGCCAACAATTCAG
AGTTTTGAACAGGTGGGAACAAAAGTGAATGTGACCGTAGAAGATGAAC
GGACTTTAGTCAGAAGGAACAACACTTTCCTAAGCCTCCGGGATGTTTT
TGGCAAGGACTTAATTTATACACTTTATTATTGGAAATCTTCAAGTTCA
GGAAAGAAAACAGCCAAAACAAACACTAATGAGTTTTTGATTGATGTGG
ATAAAGGAGAAAACTACTGTTTCAGTGTTCAAGCAGTGATTCCCTCCCG
AACAGTTAACCGGAAGAGTACAGACAGCCCGGTAGAGTGTATGGGCCAG
GAGAAAGGGGAATTCAGAGAA
(Human IL-15D8N)
AACTGGGTGAATGTAATAAGTAATTTGAAAAAAATTGAAGATCTTATT
CAATCTATGCATATTGATGCTACTTTATATACGGAAAGTGATGTTCACC
CCAGTTGCAAAGTAACAGCAATGAAGTGCTTTCTCTTGGAGTTACAAGT
TATTTCACTTGAGTCCGGAGATGCAAGTATTCATGATACAGTAGAAAAT
CTGATCATCCTAGCAAACAACAGTTTGTCTTCTAATGGGAATGTAACAG
AATCTGGATGCAAAGAATGTGAGGAACTGGAGGAAAAAAATATTAAAGA
ATTTTTGCAGAGTTTTGTACATATTGTCCAAATGTTCATCAACACTTCT

The amino acid sequence of TGFR/IL15RaSu fusion protein (including signal peptide sequence) is as follows:

(Signal peptide)
MKWVTFISLLFLFSSAYS
(Single chain Human TGF-beta Receptor II
homodimer)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPD
(Human IL-15 receptor α sushi domain)
ITCPPPMSVEHADIWVKSYSLYSRERYICNSGFKRKAGTSSLTECVLNK
ATNVAHWTTPSLKCIR

The amino acid sequence of TGFR/TF/IL15D8N fusion protein (including signal peptide sequence) is as follows:

(Signal peptide)
MGVKVLFALICIAVAEA
(Single chain Human TGF-beta Receptor II
homodimer)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPD
(Tissue factor)
SGTTNTVAAYNLTWKSTNFKTILEWEPKPVNQVYTVQISTKSGDWKSKC
FYTTDTECDLTDEIVKDVKQTYLARVFSYPAGNVESTGSAGEPLYENSP
EFTPYLETNLGQPTIQSFEQVGTKVNVTVEDERTLVRRNNTFLSLRDVF
GKDLIYTLYYWKSSSSGKKTAKTNTNEFLIDVDKGENYCFSVQAVIPSR
TVNRKSTDSPVECMGQEKGEFRE
(IL-15D8N)
NWVNVISNLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS

The TGFR/IL15RαSu and TGFR/TF/IL-15D8N constructs were cloned into a modified retrovirus expression vectors as described previously (Hughes M S, Yu Y Y, Dudley M E, Zheng Z, Robbins P F, Li Y, et al). The expression vectors were transfected into CHO-K1 cells. Co-expression of the two constructs in CHO-K1 cells allowed for formation and secretion of the soluble TGFR/IL15RαSu-TGFR/TF/IL-15D8N protein complex (referred to as TGFRt15*-TGFRs), which can be purified by anti-TF antibody affinity.

### Example 19: Binding Activity of TGFRt15-TGFRs and TGFRt15*-TGFRs to TGF-β1 and LAP

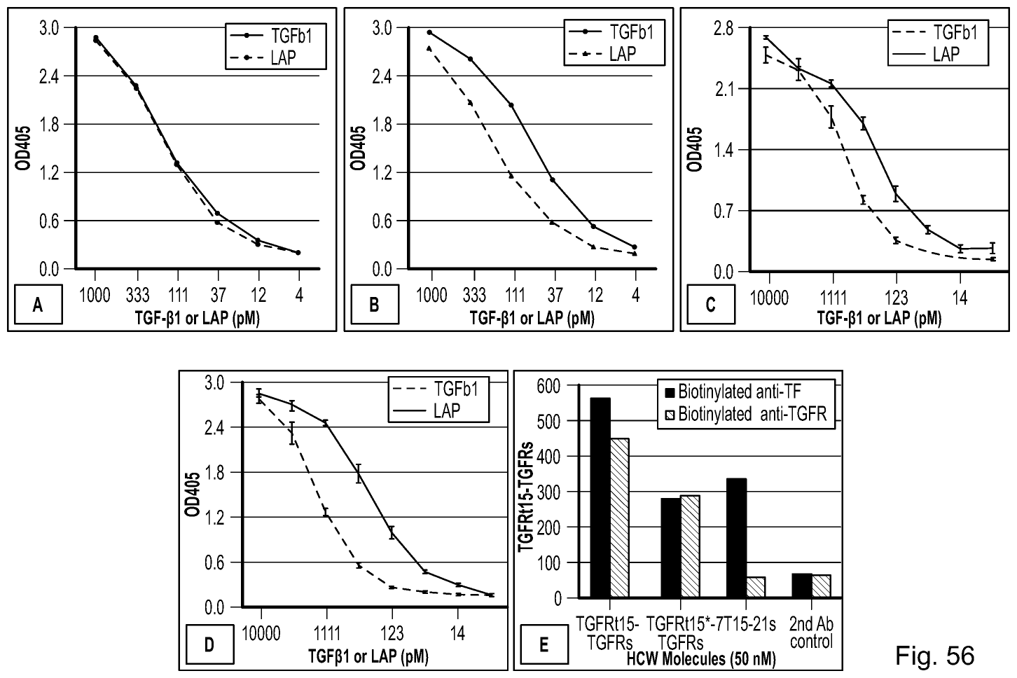

Binding activity of TGFRt15-TGFRs to TGF-β1 and LAP was determined by ELISA. TGFRt15-TGFRs (5 mg/mL) was used to capture the titrated TGF-β1 (labeled as TGFβ1, BioLegend) and latent associated peptide of TGF-β1 (LAP, R&D Systems). TGF-β1 was detected by biotinylated anti-TGF-β1 (0.2 mg/mL, R&D Systems) and LAP by biotinylated anti-LAP (0.2 mg/mL, R&D Systems) followed by peroxidase conjugated streptavidin (Jackson ImmunoResearch Lab). 2,2′-azino-bis (3-ethylbenzothiazoline-6-sulphonic acid) (ABTS, Surmodics IVD) was used as a substrate and measured by a plate reader. As shown in FIG. 56A, the results demonstrate that TGFRt15-TGFRs binds to TGF-β1 and LAP similarly, and more strongly than the Fc fusion.

Binding activity of TGF-β1 receptor/Fc fusion to TGF-β1 and LAP was determined by ELISA. A commercial TGF-β1 receptor II-Fc fusion (TGFRII/Fc) was used to compare the binding activity of TGFRt15-TGFRs to TGF-β1 and LAP. TGFRII/Fc (5 mg/mL, R&D Systems) was used to capture the titrated TGF-β1 and LAP. Other procedures were the same as described above. As shown in FIG. 56B, the results demonstrate that TGFRII/Fc binds to TGF-β1 and LAP similarly and its binding is comparable with TGFRt15-TGFRs, and stronger than the Fc fusion.

### Binding Activity of TGFRt15-TGFRs and TGFRt15*-TGFRs to TGF-β1 and LAP

TGFRt15-TGFRs and TGFRt15*-TGFRs (10 mg/mL) were used to capture the titrated TGF-β1 LAP. Other procedures were the same as described above. As shown in FIG. 56C and FIG. 56D, the results demonstrate that TGFRt15*-TGFRs binds to TGF-β1 and LAP similarly and its binding is comparable with TGFRt15-TGFRs, and stronger than the Fc fusion.

### Binding of TGFRt15-TGFRs and TGFRt15*-TGFRs to CTLL-2 Cells

IL-2-dependent CTLL-2 cells were stained with TGFRt15-TGFRs (50 nM), TGFRt15*-TGFRs (50 nM), 7t15-21s (50 nM, IL-7-TF-IL15 and IL-21-IL-15RαSu) (as a control fusion molecule, which does not contains TGF-β1 receptor II), and PBS (as a negative control) for 60 minutes and probed by biotinylated second staining antibodies (Anti-TF: anti-human tissue factor, HCW Biologics and Anti-TGFR: anti-TGF-β receptor II: R&D Systems) and then followed by R-phycoerythrin-streptavidin (Jackson ImmunoResearch Lab). The mean fluorescent intensity (MFI) of staining was measured by flow cytometry. As shown in FIG. 56E, the results show that TGFRt15-TGFRs bound to CTLL-2 cells significantly better than other molecules, TGFRt15*-TGFRs less than TGFRt15-TGFRs because of the IL-15 mutant. However, 7t15-21s binding to CTLL-2 cells could be detected with anti-TF but not anti-TGFR.

### Example 20: Biological Activities of TGFRt15-TGFRs and TGFRt15*-TGFRs with Cell-Based Assays

### TGF-β1 Blocking Activities of TGFRt15-TGFRs and TGFRt15*-TGFRs.

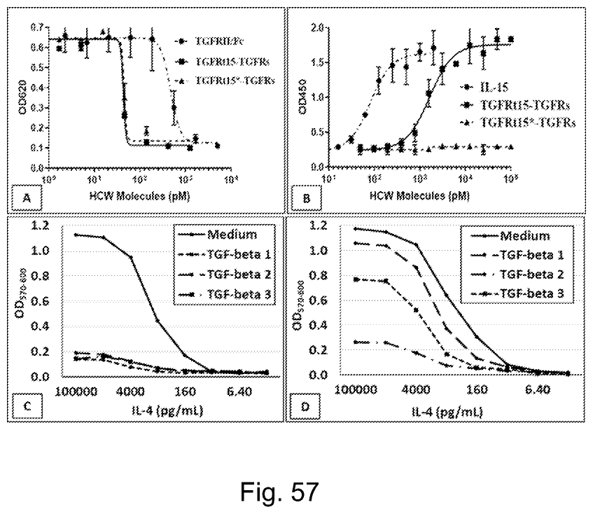

HEK-Blue TGF-β cells (InvivoGen) were incubated in IMDM-10 with titrated TGFRt15-TGFRs, TGFRt15*-TGFRs and TGFRII/Fc as a control in the presence of TGF-1 (0.1 nM, BioLegend). TGFRII/Fc is a commercial TGF-β1 receptor II-Fc fusion (R&D Systems). After 24 hours of incubation, the culture supernatants were mixed with QUANTI-Blue (InvivoGen) and incubated for 1-3 hrs. The OD620 values were measured by a plate reader. As shown in FIG. 57A, TGFRt15-TGFRs and TGFRt15*-TGFRs had the same TGF-β1 blocking activity. In contrast, TGFRII/Fc (IC50=470.2 pM) had about 10 fold lower TGF-β1 blocking activity than TGFRt15-TGFRs (IC50=43.2 pM) or TGFRt15*-TGFRs (45.2 pM). The blocking activity was calculated with GraphPad Prism 7.04.

### IL-15 Activity of TGFRt15-TGFRs and TGFRt15*-TGFRs

IL-15 dependent 32Dβ cells were cultured in IMDM-10 with titrated TGFRt15-TGFRs, TGFRt15*-TGFRs and IL15 as a control. WST-1 (Fisher Scientific) was added 2 days later and the OD450 values were measured by a plate reader. As shown in FIG. 57B, TGFRt15-TGFRs (EC50=1641 pM) had about 20 fold lower IL-15 biological activity than IL-15 itself (IC50=81.8 pM). As expected, TGFRt15*-TGFRs had no detectable IL-15 activity. The IL-15 activity was calculated with GraphPad Prism 7.04.

### Reversal of TGF-6 Growth Suppression of CTLL-2 by TGFRt15*-TGFRs

TGF-β includes three isoforms (TGF-β1, TGF-β2 and TGF-β3), which have similar biological functions. CTLL-2 cells were used to compare biological blocking activity of TGFRt15*-TGFRs in this study. TGFRt15*-TGFRs is structurally very similar to TGFRt15-TGFRs, which cannot be used to do so due to the IL-15 activity of TGFRt15-TGFRs. CTLL-2 cells were cultured in RPMI-10 with titrated mouse IL-4 (Biolegend), TGF-β (5 ng/ml, TGF-β1 (Biolegend), TGF-β2, B3 (R&D Systems)) and TGFRt15*-TGFRs (21 nM; TGFRt15*-TGFRs:TGF-β molar ratio=100:1) for 5 days. Cell proliferation (OD₅₇₀₋₆₀₀ value) was determined by a plate reader after adding PrestoBlue (Fisher Scientific) at the last day culture. FIG. 57C shows that all three TGF-β similarly inhibited IL-4 induced CTLL-2 growth in the absence of TGFRt15*-TGFRs. FIG. 57D shows that TGFRt15*-TGFRs (21 nM; TGF-β:TGFRt15*-TGFRs molar ratio=1:100) significantly reversed the inhibition of TGF-β1 and TGF-β3 of IL-4-induced CTLL-2 cell growth, In contrast, TGFRt15*-TGFRs had minimum reversal TGF-β2 inhibitory activity.

### Example 21: Stability of TGFRt15-TGFRs

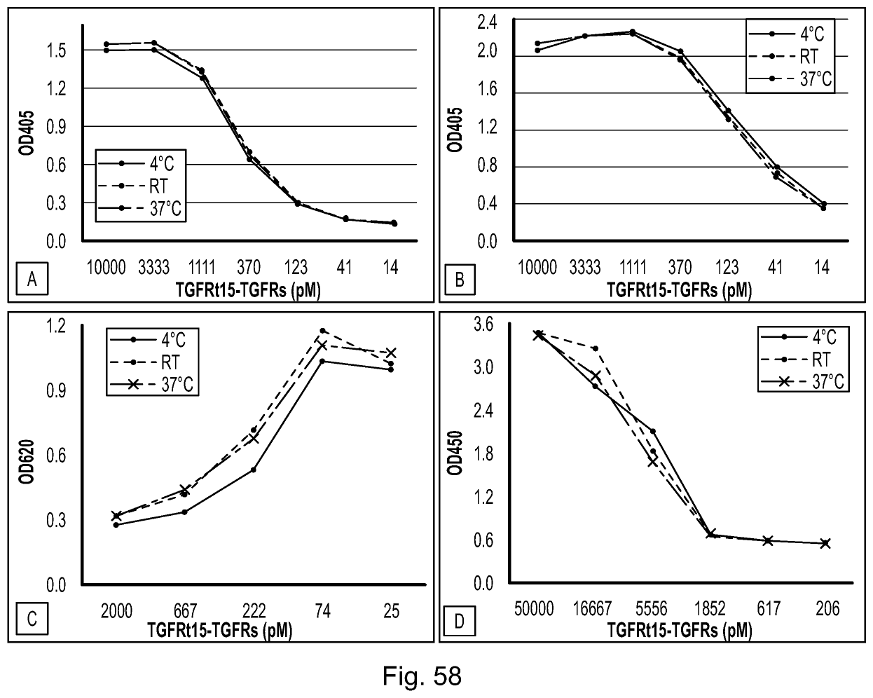

Stability of TGFRt15-TGFRs by ELISA. TGFRt15-TGFRs was preincubated in RPMI medium with 50% human serum at 4° C., room temperature (RT) or 37° C. for 10 days. IL-15 domain and TGFβRII domain of TGFRt15-TGFRs were evaluated by ELISA. Anti-TF antibody (HCW Biologics) was used to capture TGFRt15-TGFRs molecules and biotinylated anti-IL-15 (R&D Systems) was used to detect IL-15 domain and biotinylated anti-TGFβRII (R&D Systems) was used to detect TGFβRII domain. Biotinylated detection antibodies were probed by peroxidase-streptavidin (Jackson ImmunoResearch Lab). 2,2′-azino-bis(3-ethylbenzothiazoline-6-sulphonic acid) (ABTS, Surmodics IVD) was used as a substrate and OD405 value was measured by a plate reader. As shown in FIG. 58A and FIG. 58B, the results show that there were no significant changes in the domains of TGFRt15-TGFRs following 10 day incubation 4° C., RT, or 37° C. These findings demonstrate that IL-15 domain and TGFβRII domain of TGFRt15-TGFRs remain intact when incubated with human serum under the evaluated conditions.

Stability of TGFRt15-TGFRs Biological Activities with Cell-Based Assays

TGFRt15-TGFRs was preincubated in RPMI-10 with 50% human serum at 4° C., room temperature (RT) or 37° C. for 10 days. TGF-β1 neutralizing activity of TGFRt15-TGFRs was accessed with HEK-Blue TGF-β cells (TGF-β1 activity report cell line, InvivoGen). HEK-Blue TGF-β cells were incubated in IMDM-10 with titrated TGFRt15-TGFRs in the presence of TGF-β1 (0.1 nM). After 24 hours of incubation, the culture supernatants were mixed with QUANTI-Blue (InvivoGen) and incubated for 1-3 hrs. The OD620 values were measured by a plate reader. As shown in FIG. 58C, the results show that there were no changes in the TGF-β1 neutralizing activity of TGFRt15-TGFRs following incubation in human serum for 10 days at 4° C., RT, or 37° C. IL-15 activity of TGFRt15-TGFRs was evaluated with IL-15 dependent 32Dβ cells. 32Dβ cells were cultured in IMDM-10 with titrated TGFRt15-TGFRs. WST-1 (InvitroGen) was added 2 days later and the OD450 values were measured by a plate reader. As shown in FIG. 58D, the results show that there were no changes in the IL-15 activity of TGFRt15-TGFRs following incubation in human serum for 10 days at 4° C., RT, or 37° C.

### Example 22: Reversal of TGF-β1 Immunosuppression for Human NK Cells and PBMC by TGFRt15-TGFRs and TGFRt15*-TGFRs

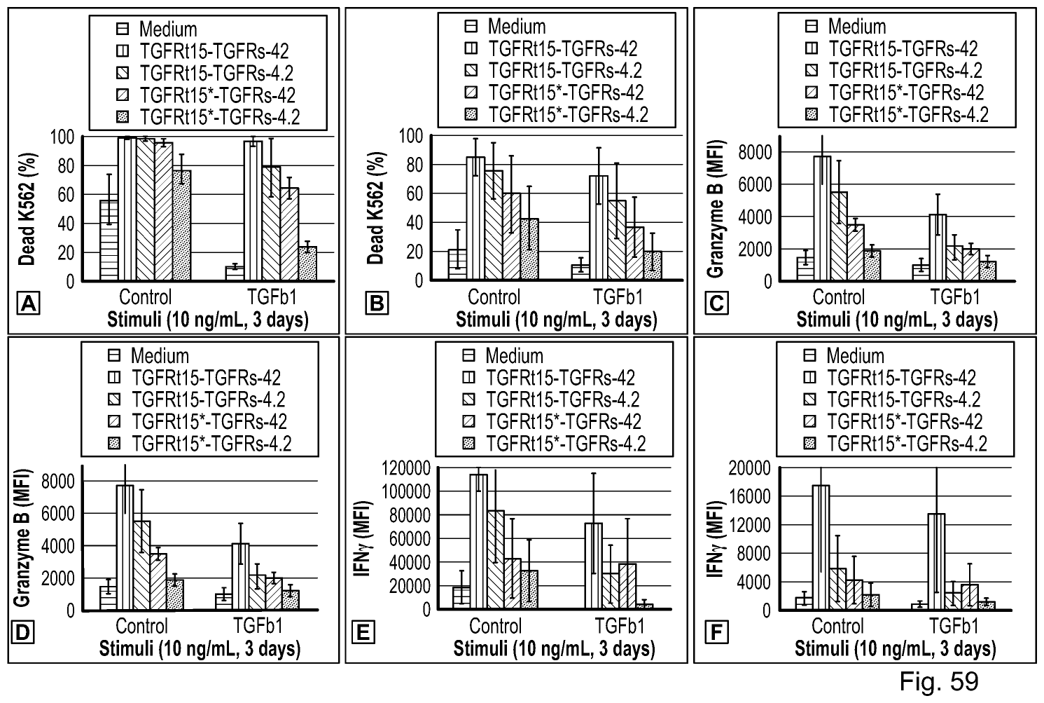

Human NK cells were purified from blood buffy coats (4 donors, One Blood) with RosetteSep™ Human NK Cell Enrichment Cocktail (StemCell) according to StemCell instruction and PBMCs were isolated from blood buffy coats (6 donors) with Ficoll-Paque (Sigma-Aldrich) density centrifugation. NK cells and PBMCs were cultured in RPMI-10 with IL-15 (10 ng/ml, PeproTech) and/or TGF-β1 (10 ng/ml, Biolegend), TGFRt15-TGFRs (42 nM or 4.2 nM) or TGFRt15*-TGFRs (42 nM or 4.2 nM) for 3 days. The cultures were harvested and used for the following assays: cell mediated cytotoxicity assay (FIGS. 59A and 59B) and flow cytometry analyses for intracellular granzyme B (FIGS. 59C and 59D) and Interferon gamma (IFNγ, FIGS. 59E and 59F).

Cultured NK cells and PBMCs were used as effector cells and K562 tumor cells (ATCC) as target cells in cell mediated cytotoxicity assay. The mixtures of the effector cells and K562 tumor cells were incubated in RPMI-10 at 37° C. for 4 hours at E:T ratio=4:1 for NK cells (FIG. 59A) or 20:1 for PBMCs (FIG. 59B). The levels of dead K562 cells were determined by flow cytometry. As shown in FIGS. 59A and 59B, the results showed that there were significantly less dead K562 target cells in the presence of TGF-β1 than were observed medium control cultures, indicating that TGF-β1 inhibits immune cell cytotoxicity. However, there were significantly more dead K562 target cells in the presence of TGF-β1 and TGFRt15-TGFRs or TGFRt15*-TGFRs than was observed cultures incubated with TGF-β1 alone conditions. These findings demonstrate TGFRt15-TGFRs and TGFRt15*-TGFRs significantly reduced TGF-β1 immunosuppression and enhanced the cytotoxicity of human NK cells and PBMCs against K562 target cells in a concentration dependent manner. Additionally, the IL-15 activity of TGFRt15-TGFRs further enhances cytotoxicity of human NK cells and PBMCs when compared to the activity of TGFRt15*-TGFRs.

Cultured NK cells and PBMCs were stained with fluorochrome labeled anti-CD56 and anti-CD16 human NK cell surface markers and then with fluorochrome-labeled granzyme B and IFNγ intracellular molecules (BioLegend). The granzyme B and IFNγ expression (MFI: mean fluorescence intensity) in the purified NK cells and gated NK cells (CD56⁺ and/or CD16⁺) of PBMC cultures were analyzed by flow cytometry. As shown in FIGS. 59C and 59D, there was significantly less granzyme B (FIGS. 59C and 59D) and IFNγ (FIGS. 59E and 59F) expression in NK cells cultured in the presence of TGF-β1 than was observed in cells cultured in medium alone, indicating that TGF-β1 inhibits immune cell activation. However, there was significantly higher granzyme B and IFNγ expression NK cells cultures in the presence of TGF-β1 and TGFRt15-TGFRs or TGFRt15*-TGFRs than was observed in cells cultured in TGF-1 alone. The TGFRt15*-TGFRs had a minimum effect on granzyme B and IFNγ expression at 4.2 nM concentration. These findings demonstrate TGFRt15-TGFRs and TGFRt15*-TGFRs significantly enhanced the granzyme B and IFNγ expression of human NK cells in a concentration-dependent manner through the activities of the IL-15 and TGFβRII domains.

### Example 23: Half-life of TGFRt15-TGFRs in C57BL/6 Mice

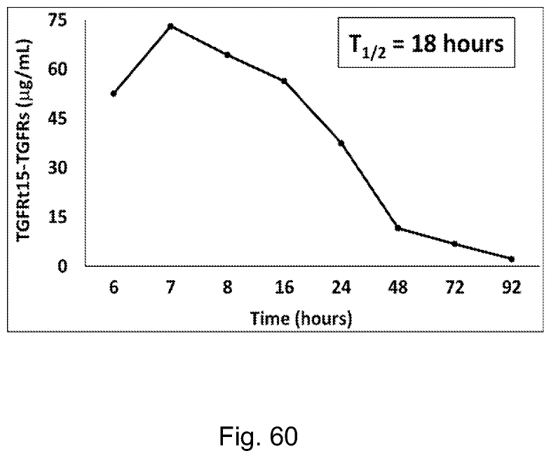

The pharmacokinetics (half-life, t½) of TGFRt15-TGFRs was evaluated in female C57BL/6 mice. The mice were treated subcutaneously with TGFRt15-TGFRs at a dosage of 3 mg/kg. The mouse blood was collected from tail vein at various time points and the serum was prepared. The TGFRt15-TGFRs concentrations in mouse serum was determined with ELISA. Anti-TF antibody (anti-human tissue factor antibody generated in HCW Biologics) was used to capture TGFRt15-TGFRs molecules and biotinylated anti-TGFβRII (R&D Systems) was used to detect TGFβRII domain. Biotinylated detection antibodies were probed by peroxidase-streptavidin (Jackson ImmunoResearch Lab). 2,2′-azino-bis(3-ethylbenzothiazoline-6-sulphonic acid) (ABTS, Surmodics IVD) was used as a substrate and the OD405 values were measured by a plate reader. As shown in FIG. 60, the half-life of TGFRt15-TGFRs was 18.22 hours in C57BL/6 mice calculated with GraphPad Prism 7.04.

### Example 24: Toxicity of TGFRt15-TGFRs in C57BL/6 Mice

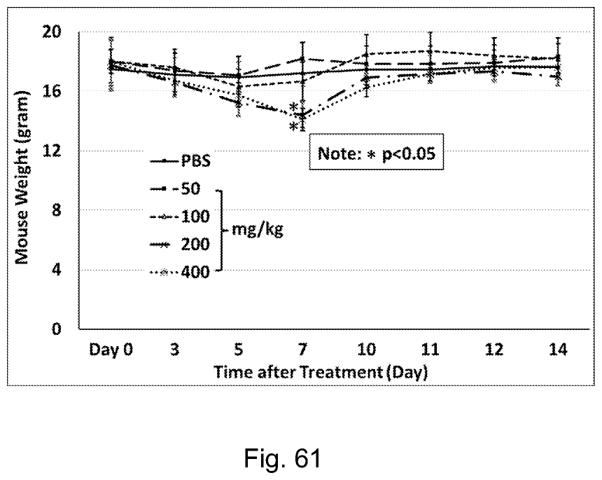

A single dose of TGFRt15-TGFRs (50-400 mg/kg) was subcutaneously injected into C57BL/6 female mice (7 weeks old, n=4). Mouse bodyweight was measured as shown in FIG. 61 and clinical signs (mortality, morbidity, ruffled fur, hunched posture, lethargy, etc.) were assessed during experimental period. The mice that received 200 mg/kg or 400 mg/kg of TGFRt15-TGFRs showed less activity 6-8 days post-treatment and without other significant clinical signs. TGFRt15-TGFRs at 200 mg/kg or 400 mg/kg caused loss in mouse body weight compared with PBS group especially on day 7 after treatment (p<0.05). The affected mice gradually recovered after 10 days without mortality or morbidity. As shown in FIG. 61, these findings indicate that C57BL/6 mice can tolerate single dose TGFRt15-TGFRs at up to 100 mg/kg.

### Example 25: Antitumor Activity of TGFRt15-TGFRs in a C57BL/6 Murine Melanoma Model

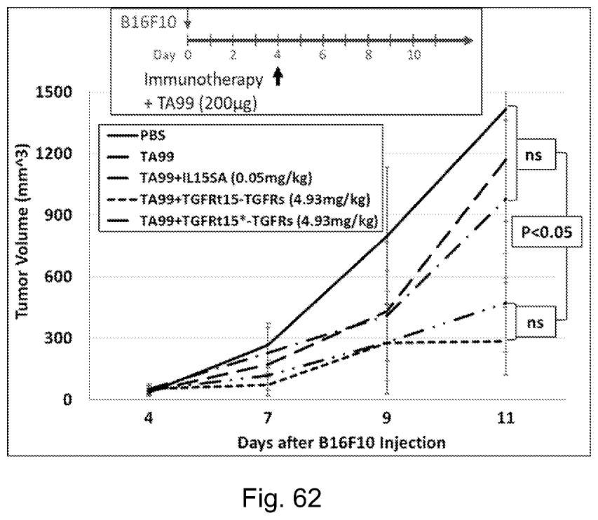

Mouse B16F10 melanoma cells were subcutaneously injected into C57BL/6 mice (The Jackson Laboratory) to establish the mouse melanoma model. Four days after tumor cell injection, the mice were divided into different groups to receive the following immunotherapies: Group 1: PBS vehicle control; Group 2: antitumor antibody TA99 (10 mg/kg) alone control; Group 3: TA99 combined with IL-15SA (0.05 mg/kg); Group 4: TA99 combined with TGFRt15-TGFRs (4.93 mg/kg, equivalent IL-15 activity of 0.05 mg/kg IL-15SA); and Group 5: TA99 combined with TGFRt15*-TGFRs (4.93 mg/kg. IL-15D8N mutant without IL-15 activity). The tumor volume was measured and calculated using the formula: length×width×width/2 formula. As shown in FIG. 62, the results indicated that the mice receiving antitumor antibody TA99 combined with TGFRt15-TGFRs or IL15SA had significantly smaller tumors at day 11 after tumor inoculation, when compared to the PBS, TA99 antibody alone, and TA99 with TGFRt15*-TGFRs groups (p<0.05). There was no significant difference among groups 1, 2, and 5 and between groups 3 and 4. These findings demonstrated that IL-15 activity of TGFRt15-TGFRs was important for antitumor activity of TGFRt15-TGFRs.

### Example 26: Model of Lung Fibrosis—Treatment with TGFRt15-TGFRs

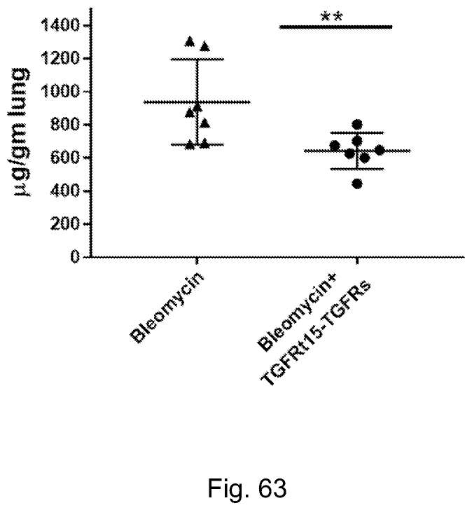

Inflammatory and fibrotic lung diseases (including idiopathic pulmonary fibrosis, chronic obstructive pulmonary disease and cystic fibrosis) are major causes of death with limited treatment options. Additionally, various therapies result in lung injury side effects leading to pulmonary fibrosis. For example, lung toxicity develops in ˜10% of cancer patients receiving bleomycin chemotherapy. These effects have led to the use of bleomycin treatment in rodents to model pulmonary fibrosis for the study of mechanisms involved in fibrogenesis and for evaluation of potential therapies. To assess the activity of TGFRt15-TGFRs in this model, nine-week old C57B16/j male mice were given 50 μL of bleomycin (2.5 mg/kg, single dose) through the oropharyngeal route. Mice were given TGFRt15-TGFRs subcutaneously (3 mg/kg) on day 17 following bleomycin treatment. Mice were sacrificed on day 28 post-bleomycin. Lungs were isolated and left lung was homogenized and 100 μL of homogenate was assayed for hydroxyproline content as a measure of collagen deposition using commercially available kit according to manufacturer's instructions. The data was expressed as μg of hydroxyproline content per gram of lung. As shown in FIG. 63, the results indicate that TGFRt15-TGFRs therapy significantly reduced collagen deposition (i.e., fibrosis) in the lungs of bleomycin-treated mice.

### Example 27: In Vivo Characterization of the Activities of TGFRt15-TGFRs and TGFRt15*-TGFRs

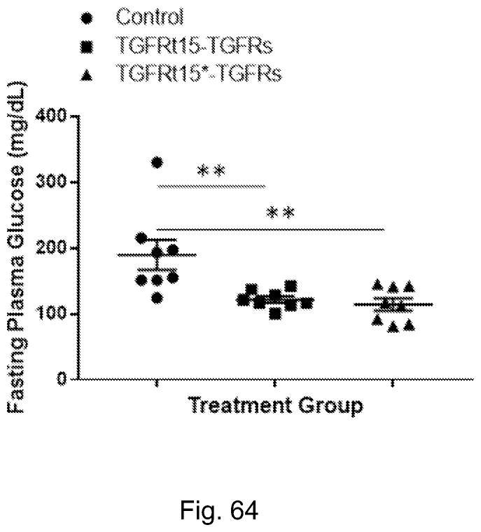

It has been shown that protection from obesity and diabetes in leptin deficient ob/ob mice can be achieved by blockade of TGF-β/Smad3 signaling. To assess if TGFRt15-TGFRs or TGFRt15*-TGFRs can protect mice from obesity and diabetes by blockade of TGF-β/Smad3 signaling, the leptin receptor deficient db/db mouse strain (BKS.Cg Dock7m⁺⁺ Leprdb/J) was used for the study. Six-week-old db/db mice were divided to three groups (N=8 per group). Mice were injected subcutaneously with TGFRt15-TGFRs, TGFRt15*-TGFRs, or PBS at 3 mg/kg. Blood was collected at day 4 post-injection through the submandibular vein after the mice had been fasting for 20 hours. The fasting blood glucose was measured with OneTouch UltraMini meter immediately after blood was drawn. As shown in FIG. 64, both TGFRt15-TGFRs and TGFRt15*-TGFRs can reduce the fasting plasma glucose levels significantly.

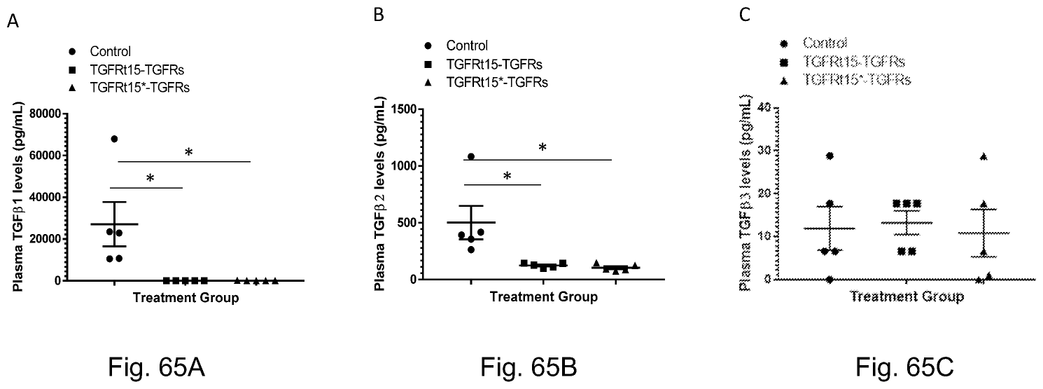

The plasma TGFβ1-3 levels were assessed to identify the cause of treatment-related reduction of fasting plasma glucose in db/db mice. Four days after treatment, plasma was isolated and 30 μL of plasma was sent to EVE Technologies (Calgary, AB Canada) to assess TGFβ1-3 levels by the TGF-3-Plex (TGFβ1-3) assay. As shown in FIGS. 65A-65C, both TGFRt15-TGFRs and TGFRt15*-TGFRs completely depleted plasma TGFβ1 (FIG. 65A), partially reduced TGFβ2 (FIG. 65B), and had no effect on TGFβ3 (FIG. 65C).

The lymphocyte subsets were assessed to identify the cause of treatment-related reduction of fasting plasma glucose in db/db mice. Four days after treatment, whole blood cells (50 μl) were treated with ACK (Ammonium-Chloride-Potassium) lysing buffer to lyse red blood cells. The lymphocytes were then stained with PE-Cy7-anti-CD3, BV605-anti-CD45, PerCP-Cy5.5-anti-CD8a, BV510-anti-CD4, and APC-anti-NKD46 (all antibodies from BioLegend) to assess the populations of T cells and NK cells. The cells were further permeabilized and fixed with eBioscience Foxp3/Transcription factor staining buffer set (Cat #00-5523-00, ThermoFisher) and stained with AF700-anti-Ki67 and FITC-anti-Granzyme B in eBioscience Permeabilization buffer (Cat #00-8333-56, ThermoFisher) to assess the proliferation and activation of T cells and NK cells. Another set of lymphocytes were stained with PE-Cy7-anti-CD3, BV605-anti-CD45, BV510-anti-CD4 and apc-Cy7-anti-CD25 first, and then permeabilized and fixed with eBioscience Foxp3/Transcription factor staining buffer set (Cat #00-5523-00, ThermoFisher) and stained with PE-anti-Foxp3 in eBioscience Permeabilization buffer (Cat #00-8333-56, ThermoFisher) to assess the population of Treg cells.

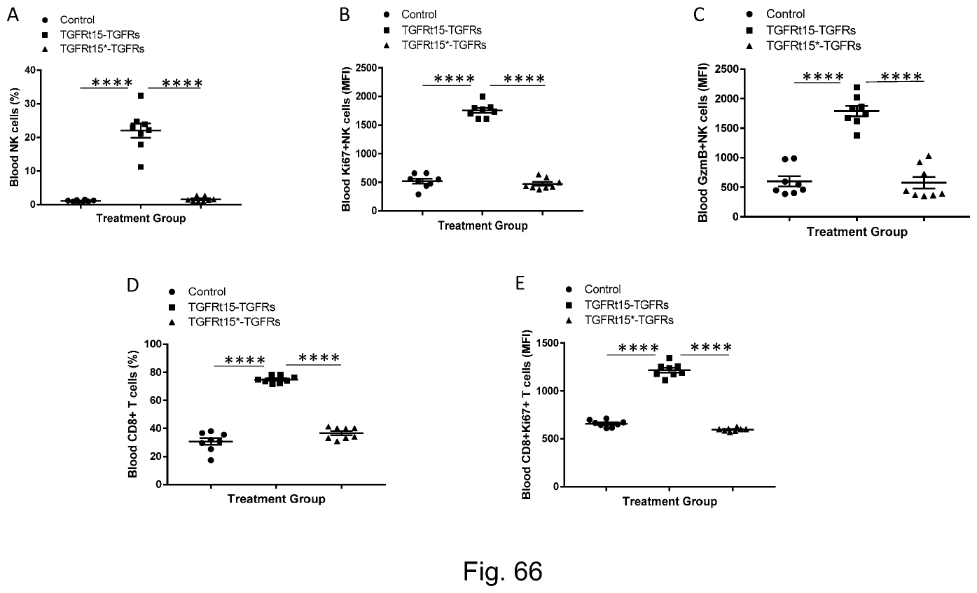

TGFRt15-TGFRs increased the population of NK cells (FIG. 66A) and CD8⁺ T cells (FIG. 66D), stimulated the proliferation of NK cells (FIG. 66B) and CD8⁺ T cells (FIG. 66E), and activated NK cells (FIG. 66C). TGFRt15*-TGFRs had no effect on either cell population (FIG. 66A-66E). Both TGFRt15-TGFRs and TGFRt15*-TGFRs had no effect on CD4⁺ T cells, CD19⁺ B cells, and CD4⁺CD25 Foxp3⁺ Treg cells.

In conclusion, in db/db mice, both TGFRt15-TGFRs and TGFRt15*-TGFRs reduced fasting plasma glucose levels and both TGFRt15-TGFRs and TGFRt15*-TGFRs completely depleted plasma TGFβ1. However, only TGFRt15-TGFRs activated NK cells and enhanced CD8⁺ T cells and NK cells proliferation. Based on these results, the depletion of TGFβ1 likely was involved in the reduction of fasting plasma glucose, showing that blockade of TGF-β/Smad3 signaling played a role in prevention of obesity and diabetes in ob/ob mice.

### Example 28: In Vitro Characterization of the Activities of TGFRt15-TGFRs and TGFRt15*-TGFRs

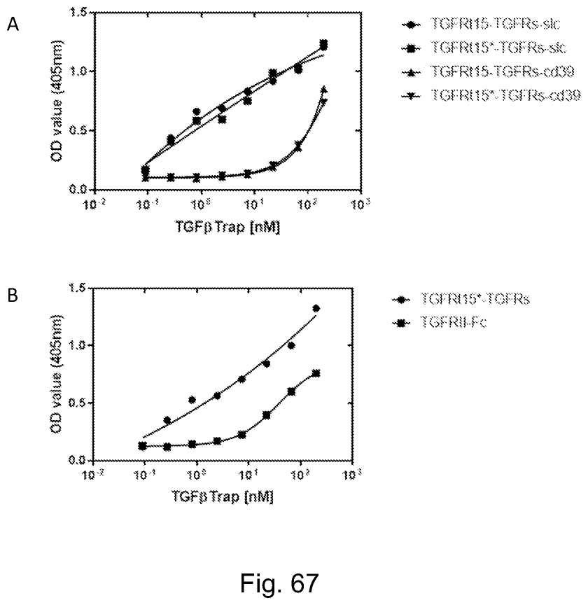

TGFRII was demonstrated to interact with TGFβ1-3. There is no report in the literature demonstrating interactions between TGFRII and latent TGFβ. To assess whether TGFRt15-TGFRs, TGFRt15*-TGFRs, and TGFRII-Fc interacts with latent TGFβ we applied 2.5 nM of human latent TGFβ1-his tag (Cat #TG1-H524x, Acro Biosystems) or a control protein CD39-his tag (Lot #58-49/51, HCW Biologics) in 50 mM carbonate buffer pH 9.4 (100 μl/well) to coat an ELISA plate (Cat #80040LE 0910, ThermoFisher) overnight at 4° C. Next day, the plate was washed with ELISA washing buffer (phosphate-buffered saline with 0.05% Tween 20) three times, the plate was blocked with the blocking buffer (1% BSA-PBS) for 1 hour, and then descending concentrations of TGFRt15-TGFRs, TGFRt15*-TGFRs, or TGFRII-Fc from 200 nM to 0.09 nM in blocking buffer were added to the plate and the plate was incubated for 1 hour at 25° C. The plate was washed three times with ELISA washing buffer. A detection antibody, biotinylated anti-TGFRII antibody (Cat #BAF241, R&D Systems), at 0.1 μg/mL was added to the plate and incubated at 25° C. for 1 hour. The plate was washed and horseradish peroxidase-streptavidin (code #016-030-084, Jackson ImmunoResearch) at 0.25 μg/mL was added to the plate and incubated at 25° C. for 30 minutes. The plate was washed and a substrate of HRP, ABTS (Cat #ABTS-1000-01, Surmodics) was added to the plate and incubated for 20 minutes at 25° C. The plate was read with a microplate reader (Multiscan Sky, Thermo Scientific) at OD405 nm. As shown in FIG. 67A, both TGFRt15-TGFRs and TGFRt15*-TGFRs interacted with latent TGFβ1 similarly. However, TGFRII-Fc interacted with latent TGFβ1 with lower affinity than was seen with TGFRt15*-TGFRs (FIG. 67B). The results demonstrated TGFRt15-TGFRs, TGFRt15*-TGFRs, and TGFRII-Fc can interact with latent TGFβ1, with TGFRt15-TGFRs, TGFRt15*-TGFRs surprisingly showing higher affinity interaction than TGFRII-Fc.

### Example 29: Prothrombin Time Test

Prothrombin time (PT) test is designed to measure the time it takes for plasma to clot after mixing with tissue factor and an optimal concentration of calcium. Tissue factor mixture with phospholipids (called Thrombinplastin) acts as an enzyme to convert prothrombin to thrombin, which in turn causes blood clotting by converting fibrinogen to fibrin. Innovin is a lipidated recombinant human TF243 and is used as the standard in our experiment. In the PT assay, shorter PT time (clotting time) indicates a higher TF-dependent clotting activity while longer PT (clotting time) means lower TF-dependent clotting activity.

Briefly, 0.1 mL of normal human plasma (Ci-Trol Coagulation Control, Level I) was prewarmed at 37° C. for 3 minutes. Plasma clotting reactions were initiated by adding 0.2 mL of various dilutions of Innovin or testing sample (TGFRt15-TGFRs) diluted in PT assay buffer (50 mM Tris-HCl, pH 7.5, 14.6 mM CaCl₂, 0.1% BSA) to the plasma. Clotting time was monitored and reported by STart PT analyzer (Diagnostica Stago, Parsippany, NJ).

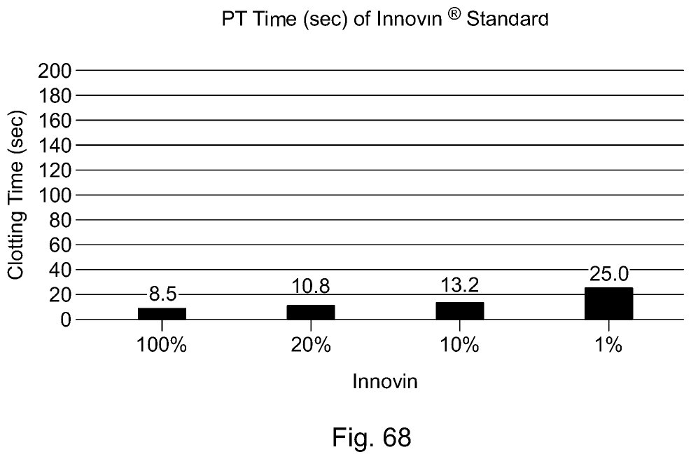

As seen in FIG. 68, different amounts of Innovin (Innovin reconstituted with purified water equivalent to 10 nM of lipidated recombinant human TF243 is considered to be 100% Innovin) added to the PT assay indeed demonstrated an inverse relationship between the amount of TF243 added in the PT assay and the PT time. For example, 1% Innovin had a PT time of about 25.0 seconds, while 100% Innovin had a PT time of 8.5 seconds.

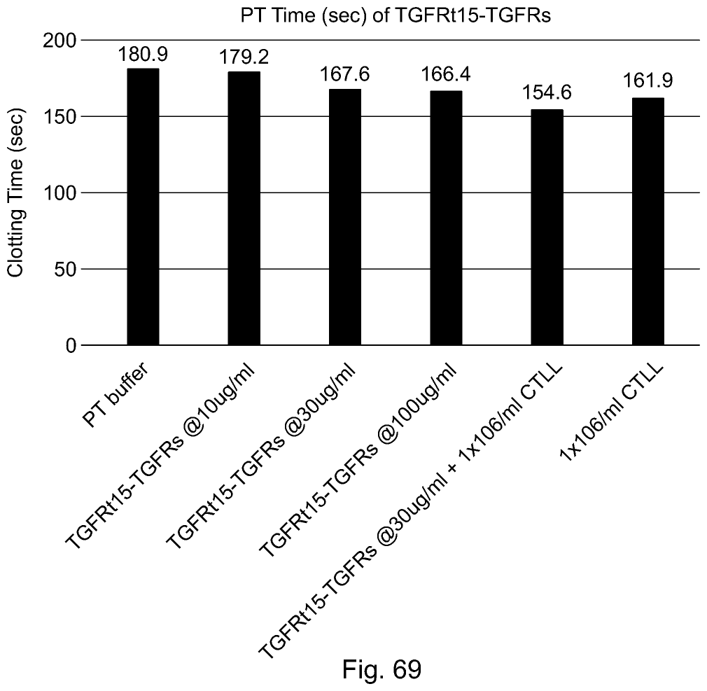

FIG. 69 shows the result of the PT test on TGFRt15-TGFRs. In contrast to Innovin, TGFRt15-TGFRs exhibited prolonged PT times which were almost the same as buffer, indicating extremely low or no clotting activity.

The clotting effect of TGFRt15-TGFRs in the presence of CTLL cells was also evaluated. The binding experiment conducted confirmed that TGFRt15-TGFRs can bind to CTLL cells. The TGFRt15-TGFRs clotting test in the presence of CTLL cells will reflect more closely with the potent clotting activity in vivo. TGFRt15-TGFRs was preincubated with CTLL cells for 20-30 min at 37° C. in PT assay buffer. Then we proceeded with the PT assay as described above. FIG. 69 shows that mixture of TGFRt15-TGFRs with CTLL cells had a bit shorter clotting time (154.6 sec) than TGFRt15-TGFRs alone (167.6 sec) or CTLL cells alone (161.9 sec). However, the clotting time of 154.6 seconds is still significantly longer than the Innovin clotting time of 8.5 seconds.

In summary, TGFRt15-TGFRs has extremely low or no TF-dependent clotting activity (i.e., in the physiological ranges of coagulation factors in human plasma), even in the presence of cells capable of binding TGFRt15-TGFRs.

### Example 30: Gene Expression of Senescence Markers in Tissues of Young Mice, and of Aged Mice Following Treatment with TGFRt15-TGFRs or PBS and Short-Term (10 Days) or Long-Term (60 Days) Follow-Up

C57BL/6, 72-week-old mice were purchased from the Jackson Laboratory. Mice were housed in a temperature and light controlled environment. Mice were divided into two groups and treated subcutaneously with either PBS (PBS control group) or TGFRt15-TGFRs at a dosage of 3 mg/kg (TGFRt15-TGFRs group). Either at day 10 or day 60 post-treatment, mice were euthanized, and kidneys were harvested in order to evaluate the expression levels of senescence markers PAI1, IL-1α, IL6, and TNFα by quantitative-PCR. Harvested kidneys were stored in liquid nitrogen in 1.7 mL Eppendorf tubes. Samples were homogenized by using homogenizer in 1 mL of Trizol (Thermo Fischer). Homogenized tissues were transferred in fresh Eppendorf tubes. Total RNA was extracted using RNeasy Mini Kit (Qiagen #74106) according to the manufacturer's instructions. One μg of total RNA was used for cDNA synthesis using the QuantiTect Reverse Transcription Kit (Qiagen). Real-time PCR was carried out with CFX96 Detection System (Bio-Rad) using FAM labeled predesigned primers purchased from Thermo Scientific. Reactions were run in triplicate for all the genes examined. The housekeeping gene 18S ribosomal RNA was used as an internal control to normalize the variability in expression levels. The expression of each target mRNA relative to 18S rRNA was calculated based on Ct as 2^{-Δ(Δct)}, in which ΔCt=Ct_{target}−Ct_{18S}. Untreated 6-week-old mice (Young) were used as a control to compare the gene expression level to aged mice.

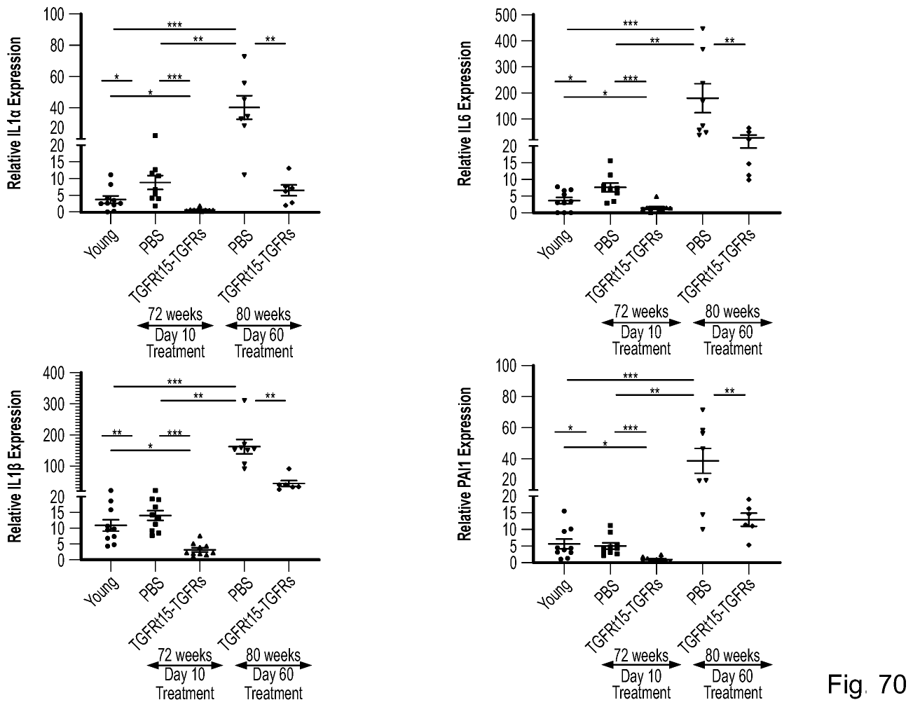

As shown in FIG. 70, the results show that gene expression of PAI-1, IL-1α, IL6, and IL-1β in kidney increased with the age of the mice as expected with the age-dependent increase in cellular senescence. Treatment of 72-month old mice with a single dose of TGFRt15-TGFRs resulted in a significant and long-lasting effect in reducing gene expression of senescence markers in kidneys, suggesting a treatment associated decrease in naturally-occurring senescent cells in the kidneys of aged mice.

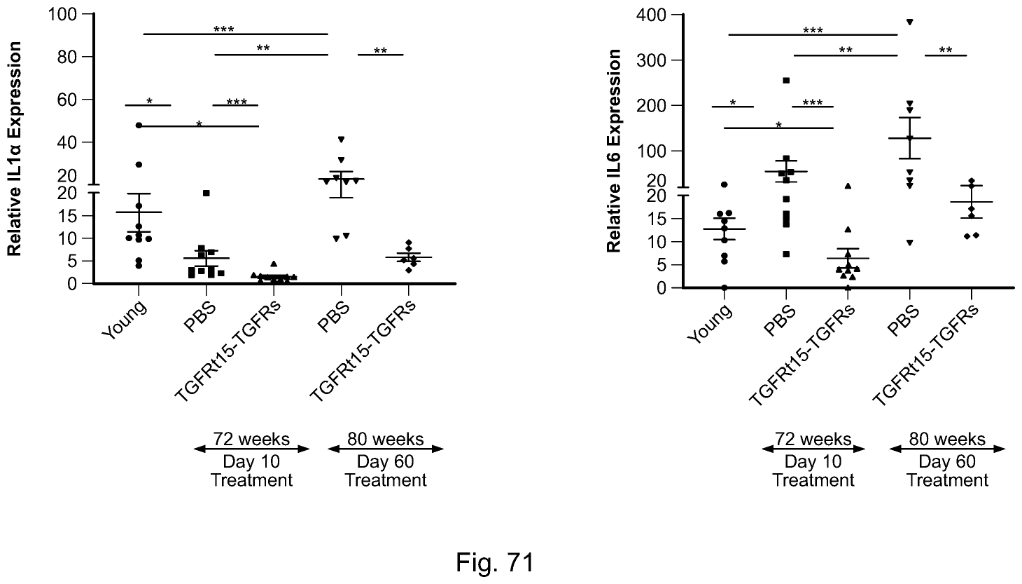

As shown in FIG. 71, the results showed that treatment of 72-month old mice with a single dose of TGFRt15-TGFRs mediated in a significant and long-lasting effect in reducing IL-1a and IL6 gene expression in liver, suggesting a treatment associated decrease in naturally-occurring senescent cells in the liver of aged mice.

C57BL/6, 72-week-old mice were purchased from the Jackson Laboratory. Mice were housed in a temperature and light controlled environment. Mice were divided into two groups and treated subcutaneously with either PBS (PBS control group) or TGFRt15-TGFRs at a dosage of 3 mg/kg (TGFRt15-TGFRs group). Either at day 10 or day 60 post-treatment, mice were euthanized, and kidneys were harvested in order to evaluate the proteins levels of the senescence marker PAI-1 by a tissue ELISA. Harvested kidneys were stored in liquid nitrogen in 1.7 mL Eppendorf tubes. Samples were homogenized by using homogenizer in 0.3 mL of extraction buffer (Abcam). Homogenized tissues were transferred in fresh Eppendorf tubes. Protein level in homogenized tissue was quantified using BCA Protein Assay Kit (Pierce). Mouse PAI-1 ELISA (R&D System) was performed with 200 mg of tissue homogenate. Based on a standard curve, the concentration of PAI-1 was calculated as picograms per milligram of tissue.

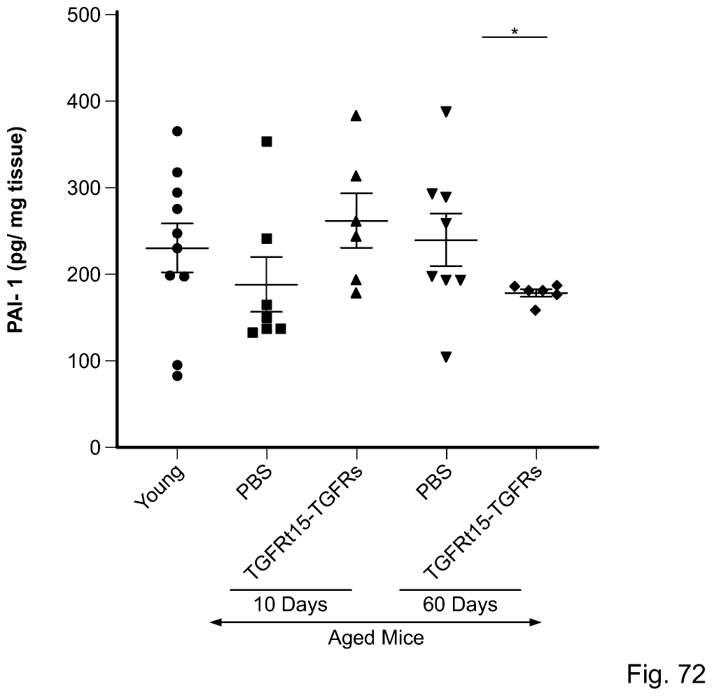

As shown in FIG. 72, the protein levels of senescence markers PAI-1 decreased in the kidneys of TGFRt15-TGFRs treated aged mice compared to PBS group at 60 days post-treatment. These results are consistent with the effects of TGFRt15-TGFRs treatment on the PAI-1 gene expression in the kidneys of aged mice. Together, these results indicate that a single treatment of TGFRt15-TGFRs resulted in a significant and long-lasting effect in reducing naturally-occurring senescent cells (as measured by reduced gene and protein expression of senescence markers) in the tissues of aged mice.

### Example 31: Comparison of TGFRt15-TGFRs and TGFRt15*-TGFRs (IL-15 Mutant) Treatment in Reducing Gene Expression of Senescence Markers in Tissues of Aged Mice

C57BL/6, 72-week-old mice were purchased from the Jackson Laboratory. Mice were housed in a temperature and light controlled environment. Mice were divided into five groups as follows: saline control group (n=8); TGFRt15-TGFRs group (n=8); IL15SA group (n=8); TGFRt15*-TGFRs group (n=8); and IL15SA+TGFRt15*-TGFRs group (n=8). Mice were treated subcutaneously with PBS, TGFRt15-TGFRs (3 mg/kg), TGFRt15*-TGFRs (3 mg/kg), IL15SA (0.5 mg/kg), or TGFRt15*-TGFRs (3 mg/kg) plus IL15SA (0.5 mg/kg). Mouse blood was prepared in order to evaluate changes in the different subsets of immune cells after treatment with TGFRt15-TGFRs and other agents. The mouse blood was collected from submandibular vein on Day 17 post-treatment in tubes containing EDTA. The whole blood was centrifuged to collect plasma at 3000 RPM for 10 minutes in a micro centrifuge. Plasma was stored at −80° C. and whole blood was processed for immune cell phenotyping by flow cytometry. Whole blood RBCs were lysed in ACK buffer for 5 minutes at room temperature. Remaining cells were washed in FACS buffer (1×PBS (Hyclone) with 0.5% BSA (EMD Millipore) and 0.001% Sodium Azide (Sigma)). To assess the different types of immune cells in blood, cells were stained with antibodies specific to cell-surface CD3, CD45, CD8, and NK1.1 (BioLegend) for 30 minutes at room temperature (RT). After surface staining, cells were washed (1500 RPM for 5 minutes at room temperature) in FACS buffer (1×PBS (Hyclone) with 0.5% BSA (EMD Millipore) and 0.001% Sodium Azide (Sigma)). After two washes, cells were resuspended in fixation buffer and analyzed by flow cytometry (Celesta-BD Bioscience).

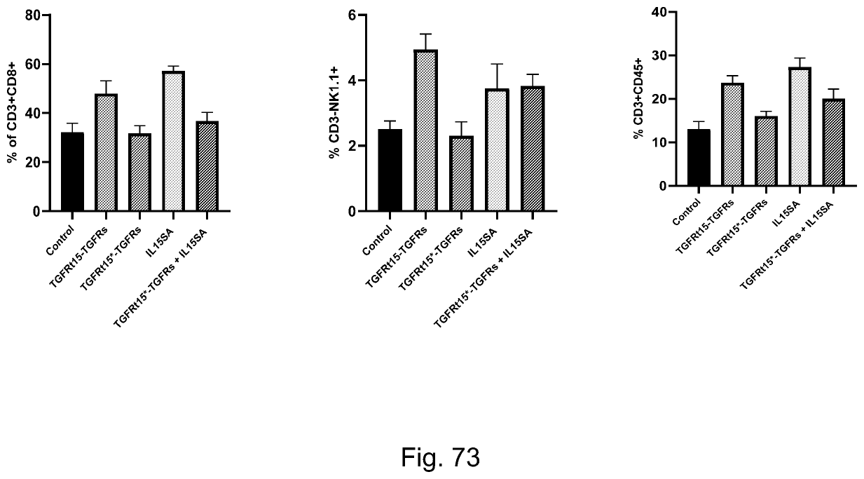

As shown in FIG. 73, the results indicate that treatment of aged mice with TGFRt15-TGFRs. IL15SA (positive control) or TGFRt15*-TGFRs+IL15SA mediated an increase in the percentages of CD3⁺CD8⁺, CD3⁺NK1.1⁺, and CD3⁺CD45⁺ immune cells in the blood, whereas treatment with TGFRt15*-TGFRs had little or no effect on the percentage of these cell populations. These results suggest that IL-15 activity of TGFRt15-TGFRs plays a role in increasing CD8⁺ T cells and NK cells in the blood of aged mice.

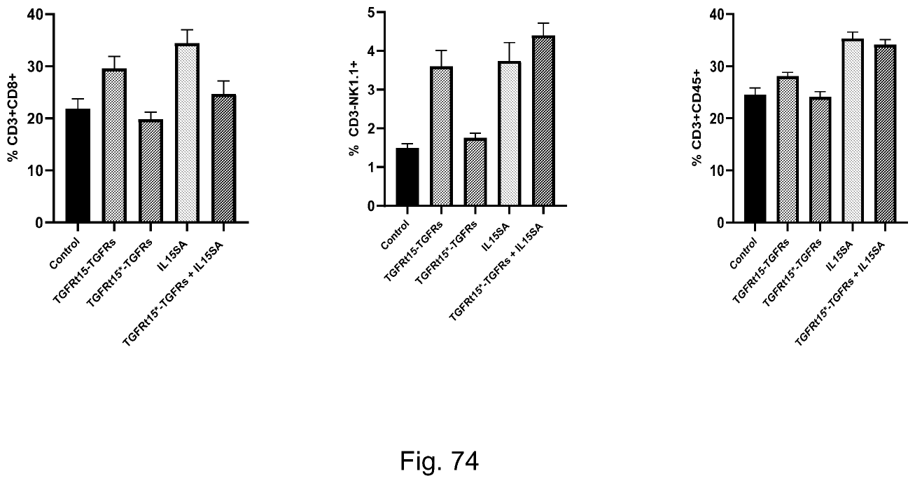

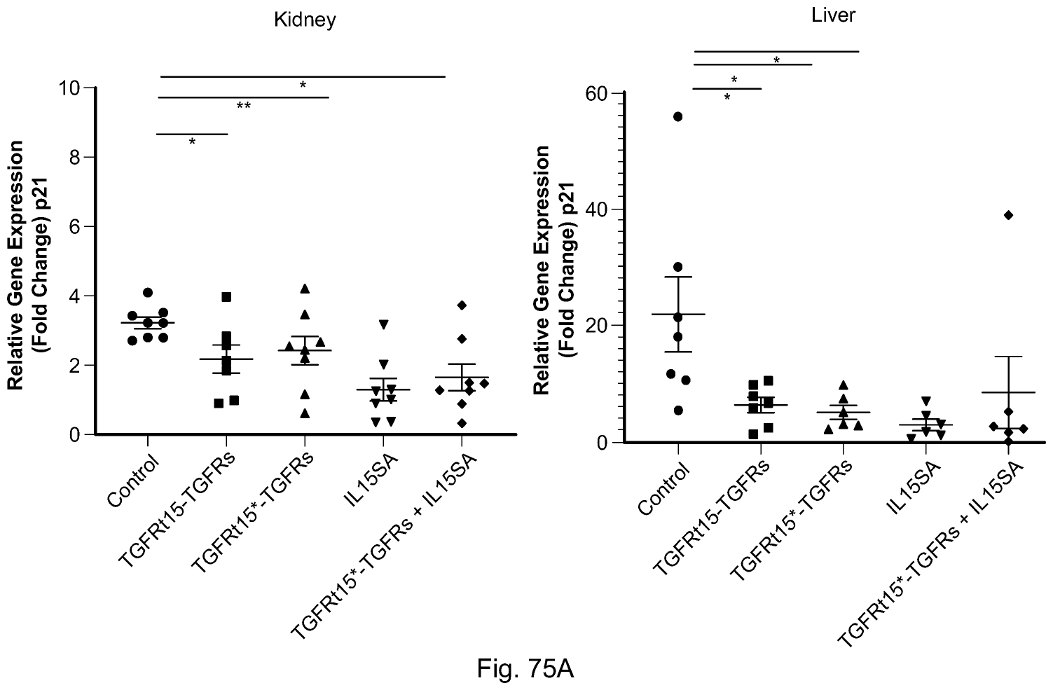

As shown in FIG. 74, the results indicate that treatment of aged mice with TGFRt15-TGFRs. IL15SA (positive control) or TGFRt15*-TGFRs+IL15SA mediated an increase in the percentages of CD3⁺CD8⁺, CD3⁻NK1.1⁺, and CD3⁺CD45⁺ immune cells in the spleen, whereas treatment with TGFRt15*-TGFRs had little or no effect on the percentage of these cell populations. These results suggest that IL-15 activity of TGFRt15-TGFRs plays a role in increasing CD8⁺ T cells and NK cells in the spleen of aged mice.

C57BL/6, 72-week-old mice were purchased from the Jackson Laboratory. Mice were housed in a temperature and light controlled environment. Mice were divided into five groups as follows: saline control group (n=8); TGFRt15-TGFRs group (n=8); IL15SA group (n=8); TGFRt15*-TGFRs group (n=8); and IL15SA with TGFRt15*-TGFRs group (n=8). Mice were treated subcutaneously with PBS, TGFRt15-TGFRs (3 mg/kg), TGFRt15*-TGFRs (3 mg/kg), IL15SA (0.5 mg/kg), or TGFRt15*-TGFRs (3 mg/kg) plus IL15SA (0.5 mg/kg). The mouse kidney, liver, and lungs were harvested in order to evaluate the gene expression of senescence markers p21, PAI1, IL-1α, and IL6 by quantitative-PCR in tissues after treatment with TGFRt15-TGFRs, TGFRt15*-TGFRs, or control groups. Mice were euthanized day 17 post-treatment and kidney, liver, and lung were harvested and stored in liquid nitrogen in 1.7 mL Eppendorf tubes. Samples were homogenized by using homogenizer in 1 mL of Trizol (Thermo Fischer). Homogenized tissues were transferred in fresh Eppendorf tubes. Total RNA was extracted using RNeasy Mini Kit (Qiagen #74106) according to the manufacturer's instructions. One μg of total RNA was used for cDNA synthesis using the QuantiTect Reverse Transcription Kit (Qiagen). Real-time PCR was carried out with CFX96 Detection System (Bio-Rad) using FAM labeled predesigned primers purchased from Thermo Scientific. Reactions were run in triplicate for all the genes examined. The housekeeping gene 18S ribosomal RNA was used as an internal control to normalize the variability in expression levels. The expression of each target mRNA relative to 18S rRNA was calculated based on Ct as 2^{-Δ(Δct)}, in which ΔCt=Ct_{target}−Ct_{18S}.

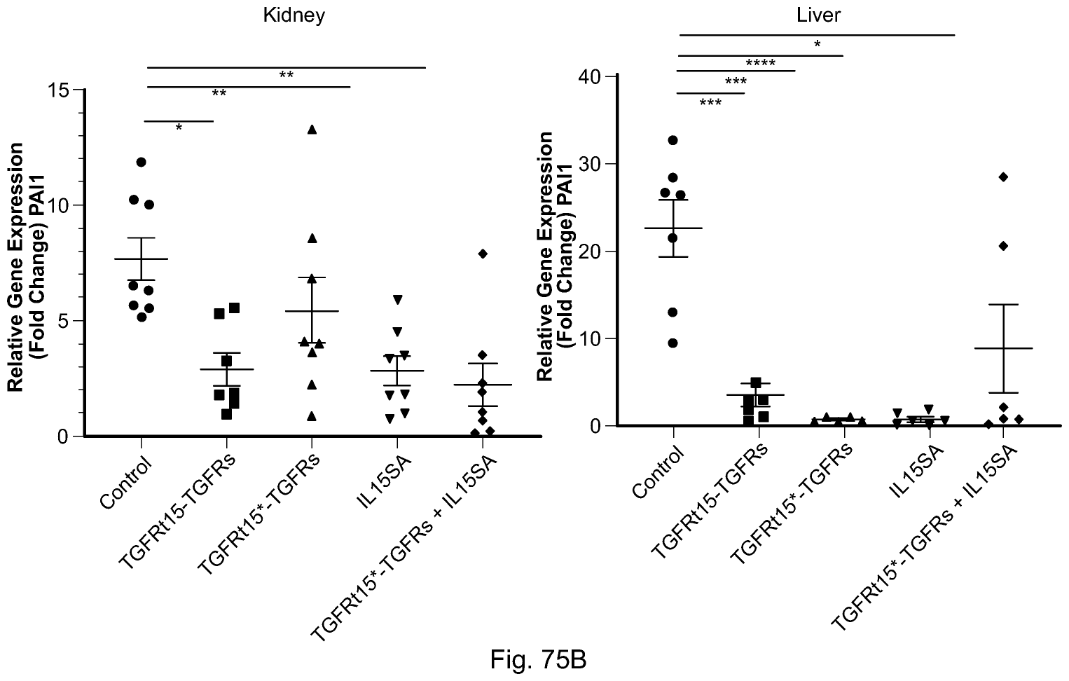

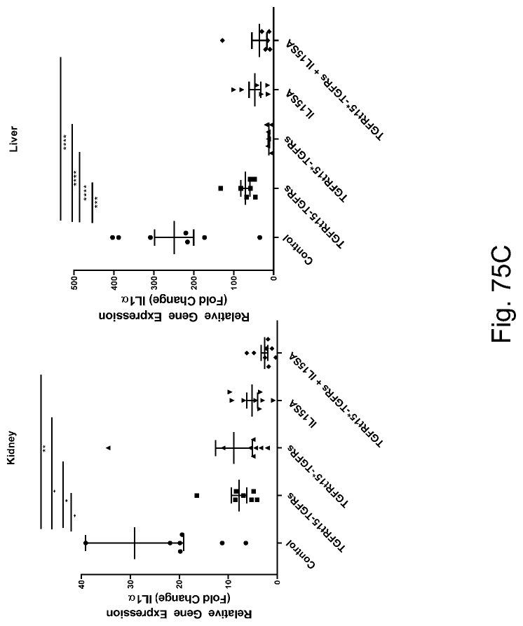

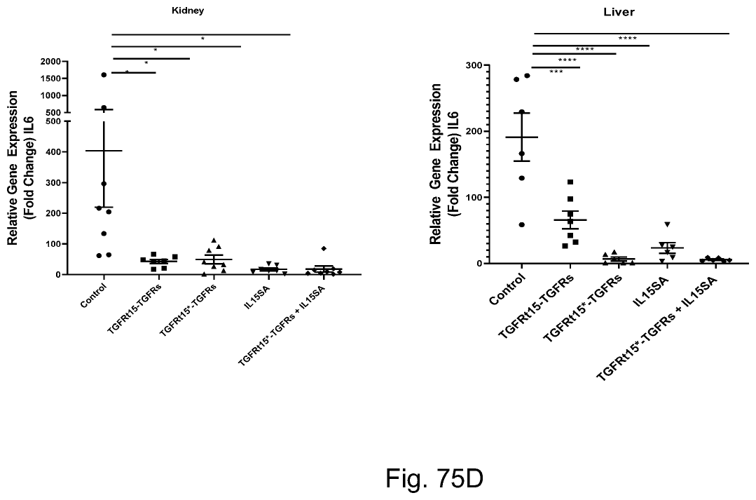

As shown in FIG. 75A-D, treatment of 72-month old mice with a single dose of TGFRt15-TGFRs or TGFRt15*-TGFRs mediated in a significant decrease in p21, PAI1, IL-1α, and IL6 gene expression in kidney and liver, suggesting a treatment associated decrease in naturally-occurring senescent cells in the kidney and liver of aged mice. The results of this study suggest that both the IL-15 and TGF-β trap activities of TGFRt15-TGFRs are capable of reducing naturally-occurring senescent cells in the tissues of aged mice.

### Example 32: Immuno-Phenotype Following Treatment with IL-15-Based Agents

The mouse blood was prepared in order to evaluate changes in the different subsets of immune cells after treatment with IL-15-based agents: TGFRt15-TGFRs, an IL-15 superagonist (IL-15SA), and an IL-15 fusion with a D8N mutant knocking out the IL-15 activity (TGFRt15*-TGFRs). C57BL/6, 6-week-old mice were purchased from Jackson Laboratory. Mice were housed in a temperature and light controlled environment. Mice were divided into groups (n=6/group) and treated with the following: 1) PBS (saline) control, 2) docetaxel, 3) docetaxel with TGFRt15-TGFRs, 4) docetaxel with IL15SA, 5) docetaxel with an IL-15 mutant (TGFRt15*-TGFRs), and 6) docetaxel with an IL-15 superagonist (IL-15SA) plus TGFRt15*-TGFRs. Senescence was induced in mice with three doses of docetaxel (10 mg/kg) at day 1, 4, and 7. On day 8, the mice were treated subcutaneously with PBS, TGFRt15-TGFRs, TGFRt15*-TGFRs, IL-15SA or in combinations as discussed above. TGFRt15-TGFRs and TGFRt15*-TGFRs were administered at a dosage of 3 mg/kg and IL-15SA was administered at 0.05 mg/kg. The mouse blood was collected from the submandibular vein on day 3 post-study drug treatment into EDTA tubes. The whole blood was centrifuged to collect plasma at 3000 RPM for 10 minutes in a microcentrifuge. Plasma was stored at −80° C. and whole blood was processed for immune cell phenotyping by flow cytometry. RBCs were lysed in ACK buffer for 5 minutes at 37° C. The remaining cells were washed in FACS buffer (1×PBS (Hyclone) with 0.5% BSA (EMD Millipore) and 0.001% Sodium Azide (Sigma)). To assess the different types of immune cells in the blood, cells were stained with antibodies for cell-surface CD4, CD45, CD19, CD8, and NK1.1 (BioLegend) for 30 minutes at room temperature (RT). After surface staining, cells were washed (1500 RPM for 5 minutes at room temperature) in FACS buffer (1×PBS (Hyclone) with 0.5% BSA (EMD Millipore) and 0.001% Sodium Azide (Sigma)). The cells were treated with permeabilization buffer (Invitrogen) for 20 minutes at 40° C. followed by wash with permeabilization buffer (Invitrogen). The cells were then stained for an intracellular marker for proliferation (Ki67) for 30 minutes at RT. After two washes, the cells were resuspended in fixation buffer and analyzed by flow cytometry (Celesta-BD Bioscience).

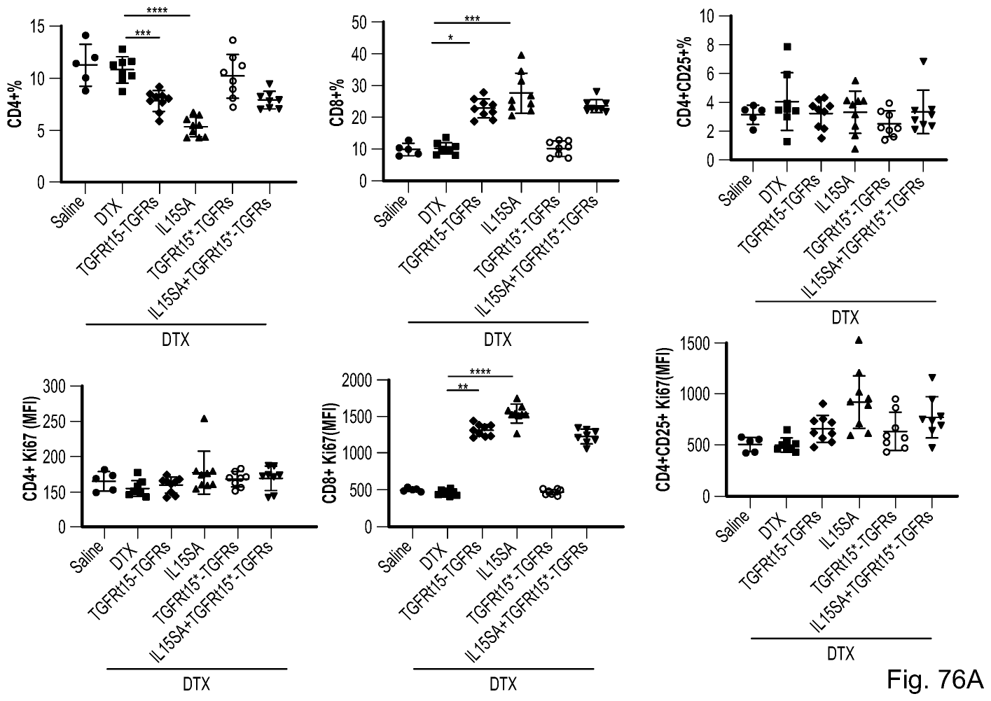

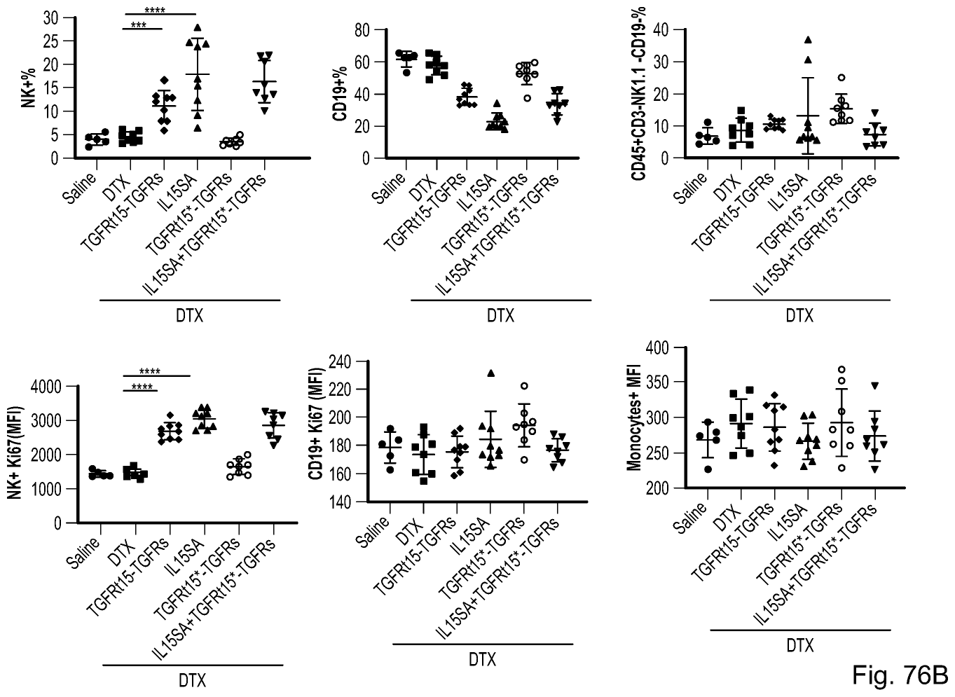

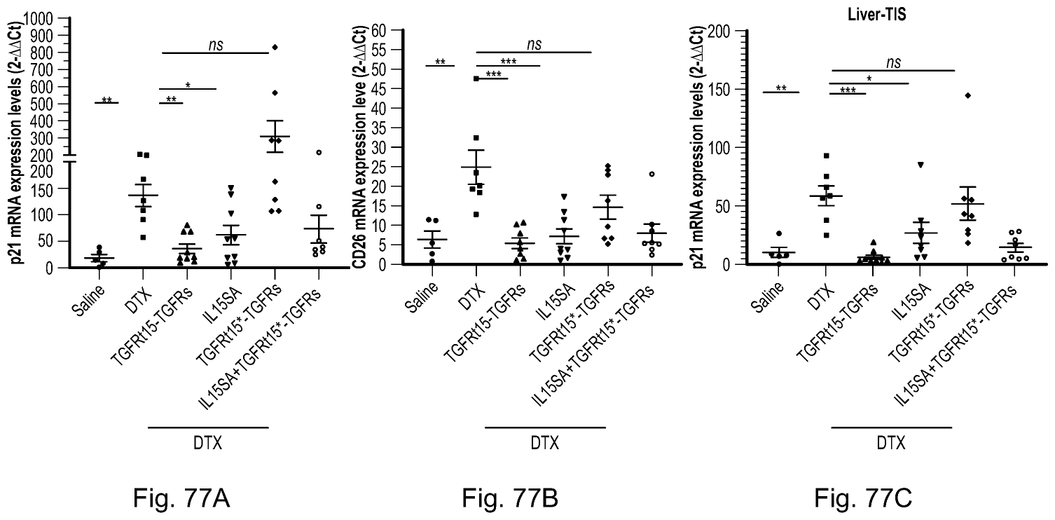

As shown in FIGS. 76A and 76B, the results indicate that treatment of mice with TGFRt15-TGFRs, IL15SA (positive control), or TGFRt15*-TGFRs+IL15SA mediated an increase in the percentages and proliferation (as measured by Ki67) of CD8⁺ T cells and NK1.1⁺ cells in the blood, whereas treatment with TGFRt15*-TGFRs had little or no effect on the percentage of these cell populations. These results suggest that IL-15 activity of TGFRt15-TGFRs plays a role in increasing CD8⁺ T cells and NK cells in the blood of mice following chemotherapy.

### Example 33: Evaluation of Gene Expression of Senescence Markers p21 and CD26 in Lung and Liver Tissues of Mice Following Chemotherapy and Treatment with IL-15-based Agents

Gene expression of markers for cell senescence were evaluated in tissues of normal mice following chemotherapy and administration of study treatments. C57BL/6, 6-week-old mice were purchased from Jackson Laboratory. Mice were housed in a temperature and light controlled environment. Mice were divided into six groups and treated with the following: 1) PBS (saline) control (n=5), 2) docetaxel (n=8), 3) docetaxel with TGFRt15-TGFRs (n=8), 4) docetaxel with IL15SA (n=8), 5) docetaxel with an IL-15 mutant (TGFRt15*-TGFRs) (n=8), and 6) docetaxel with an IL-15 superagonist (IL-15SA) plus TGFRt15*-TGFRs (n=6). Senescence was induced in mice with three doses of docetaxel (10 mg/kg) at day 1, 4, and 7. On day 8, the mice were treated subcutaneously with PBS, TGFRt15-TGFRs, TGFRt15*-TGFRs, IL-15SA, or in combinations as discussed below. TGFRt15-TGFRs and TGFRt15*-TGFRs were administered at a dosage of 3 mg/kg and IL-15SA was administered at 0.5 mg/kg. The mouse tissues were prepared in order to evaluate the different gene expression of senescence markers. Mice were euthanized on day 7 post-study drug treatment and the liver and lung tissues were harvested and stored in liquid nitrogen in 1.7 mL Eppendorf tubes. Samples were homogenized by using mortar and pestle in liquid nitrogen. Homogenized tissues were transferred in fresh Eppendorf tubes containing 1 mL of Trizol (Thermo Fischer). Total RNA was extracted using RNeasy Mini Kit (Qiagen #74106) according to the manufacturer's instructions and 1 μg of total RNA was used for cDNA synthesis using the QuantiTect Reverse Transcription Kit (Qiagen). Real-time PCR was carried out with CFX96 Detection System (Bio-Rad) using FAM labeled predesigned primers purchased from Thermo Scientific. Reactions were run in triplicate for all the genes examined. The housekeeping gene 18S ribosomal RNA was used as an internal control to normalize the variability in expression levels. The expression of each target mRNA relative to 18S rRNA was calculated based on Ct as 2^{-Δ(Δct)}, in which ΔCt=Ct target−Ct18S.

As shown in the FIGS. 77A-77C, gene expression of the senescence markers p21 and CD26 was induced in the lung (FIG. 77A) and (FIG. 77B), and p21 in liver (FIG. 77C) tissues of mice treated with docetaxel, as compared to gene expression in tissue of saline-treated mice. Gene expression of senescence markers p21 and CD26 in the lungs and p21 in the liver were reduced of the chemotherapy-treated mice following subsequent treatment with TGFRt15-TGFRs, IL-15SA, and combination of IL-15SA and TGFRt15*-TGFRs mutant, as compared to the chemotherapy-treated controls. However, the TGFRt15*-TGFRs mutant treatment failed to affect the chemotherapy-induced senescence marker gene expression in these tissues. These results show that IL-15 activity is important for clearance of TIS senescence cells in normal tissues of mice.

### Example 34: TGFRt15-TGFRs Treatment Enhances the Immune Cell Proliferation, Expansion, and Activation in the Peripheral Blood of B16F10 Tumor Bearing Mice

C57BL/6 mice were subcutaneously injected with 0.5×10⁶ B16F10 cells. After tumor inoculation (day 0), the mice were given three doses of doxetaxel chemotherapy (10 mg/kg) on days 1, 4, and 7 and single dose of TGFRt15-TGFRs (3 mg/kg) combined with monoclonal antibody targeting a tumor antigen anti-TYRP-1 antibody TA99 (200 μg) on day 8. Tumor-bearing mice treated with saline or doxetaxel chemotherapy (10 mg/kg) on days 1, 4, and 7 served as controls. Blood was drawn from submandibular vein on days 3, 5, and 10 after immunotherapy treatment (day 8). The RBCs were lysed in ACK lysis buffer and the lymphocytes were washed and stained with antibodies specific to cell-surface expression of NK, CD8, CD25, and Granzyme B (GzB) (BioLegend) for 30 minutes at room temperature (RT). After surface staining, the cells were washed (1500 RPM for 5 minutes at RT) in FACS buffer (1×PBS (Hyclone) with 0.5% BSA (EMD Millipore) and 0.001% Sodium Azide (Sigma)). After two washes, the cells were resuspended in fixation buffer. After fixation, the cells were washed and treated with permeabilization buffer (Invitrogen) for 20 minutes at 4° C. followed by wash with permeabilization buffer (Invitrogen). The cells were then stained for an intracellular marker for proliferation (Ki67) for 30 minutes at RT. After two washes, the cells were resuspended in fixation buffer and analyzed by flow cytometry (Celesta-BD Bioscience).

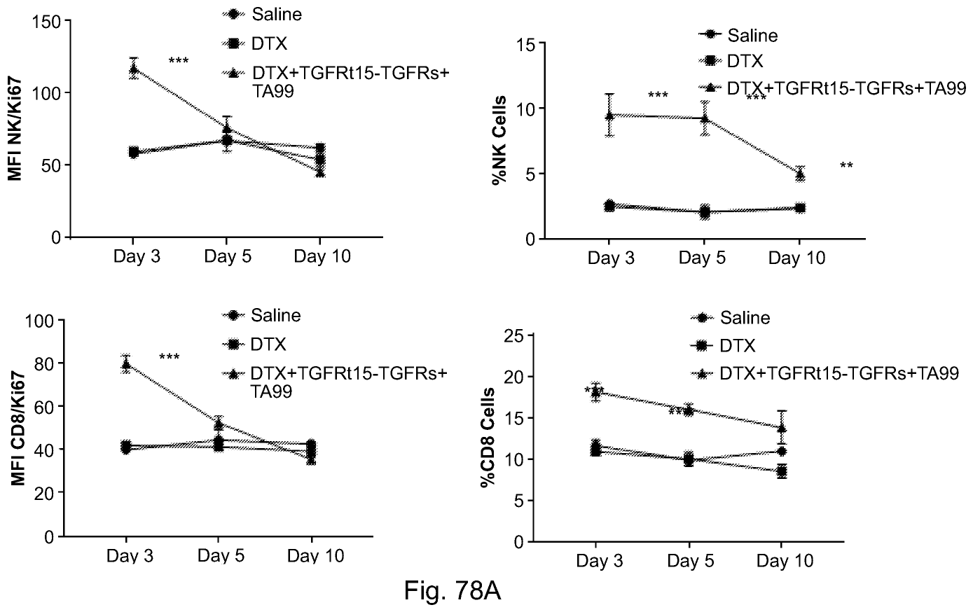

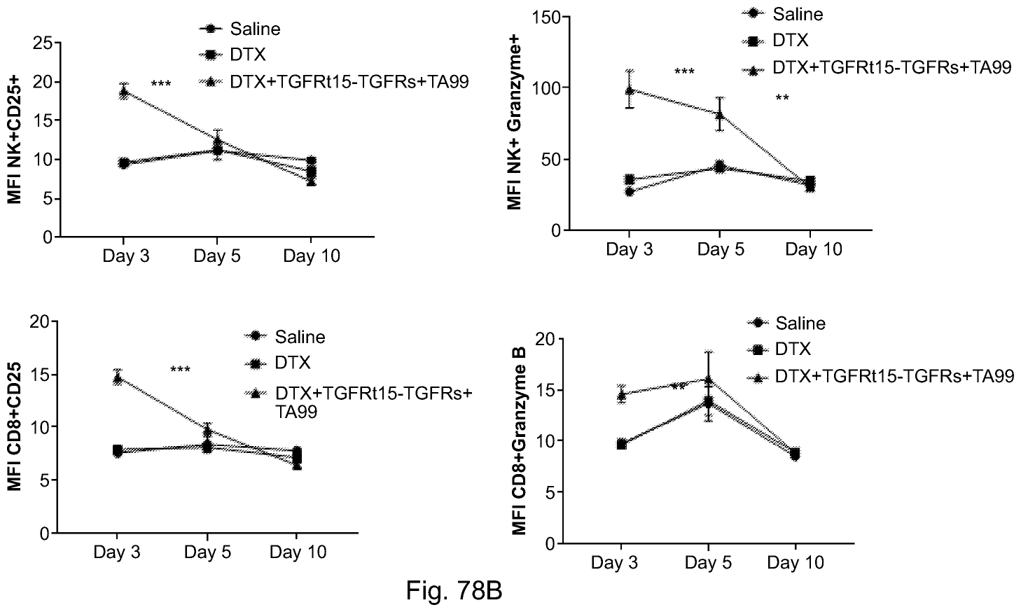

As shown in FIGS. 78A and 78B, peripheral blood analysis showed that proliferative Ki67-positive NK and CD8+ cells were predominantly present at day 3 post-TGFRt15-TGFRs+TA99 therapy, when compared to the saline or chemotherapy treatment groups. The expansion of NK and CD8+ cells was found on days 3 and 5 post-immunotherapy. While the NK cells were still expanding, the CD8+ cells was not found to be expanding in the blood at day 10 post-immunotherapy. These cells also expressed the activation markers CD25 and granzyme B post-TGFRt15-TGFRs+TA99 therapy, when compared to immune cells of the saline or chemotherapy treatment groups. These effects are consistent with the immunostimulatory activities of TGFRt15-TGFRs.

### Example 35: TGFRt15-TGFRs Treatment Decreases Levels of TGFβ in the Plasma of B16F10 Tumor Bearing Mice

C57BL/6 mice were subcutaneously injected with 0.5×10⁶ B16F10 cells. After tumor inoculation (day 0), the mice were given three doses of doxetaxel chemotherapy (10 mg/kg) on days 1, 4, and 7 and single dose of TGFRt15-TGFRs (3 mg/kg) combined with monoclonal antibody targeting a tumor antigen anti-TYRP-1 antibody TA99 (200 μg) on day 8. Tumor-bearing mice treated with saline or doxetaxel chemotherapy (10 mg/kg) on days 1, 4, and 7 served as controls. Blood was collected from the submandibular on days 1, 3, 5, and 10 after immunotherapy treatment in tubes containing EDTA and immediately placed on ice. The blood was centrifuged for 15 minutes at 3,000 rpm at room temperature to separate plasma. Plasma samples were aliquoted and stored at −80° C. The plasma TGFβ levels were analyzed by using cytokine array, TGFβ 3-plex (TGFβ 1-3) from Eve Technologies, Calgary, AL, Canada.

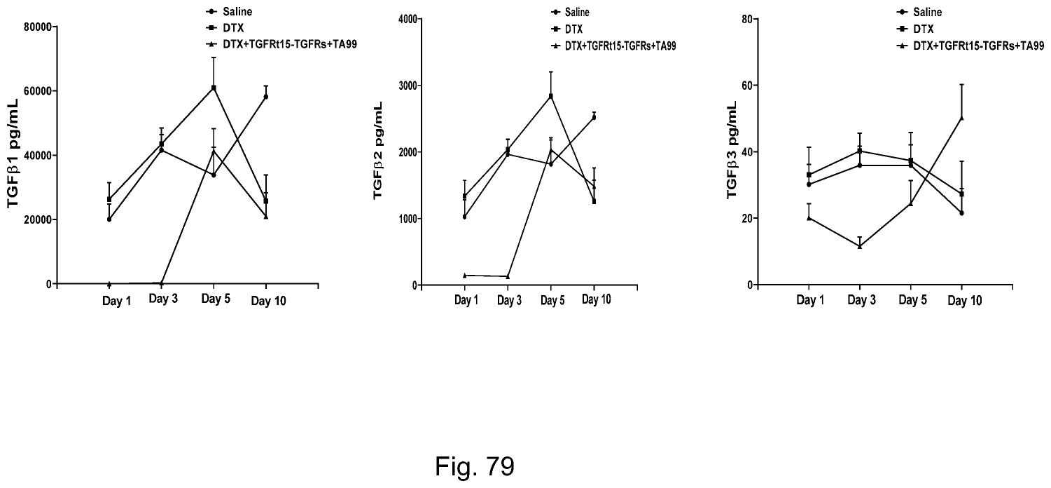

As shown in FIG. 79, the results show that administration of TGFRt15-TGFRs+TA99 led to a reduction in the plasma levels of TGF-β1, TGF-β2, and TGF-β3 in tumor-bearing mice for 3 to 5 days post-treatment, when compared to the saline or chemotherapy treatment groups. This effect is consistent with the TGF-agonistic activity of TGFRt15-TGFRs.

### Example 36: TGFRt15-TGFRs Treatment Reduces Levels of Proinflammatory Cytokines in the Plasma of B16F10 Tumor Bearing Mice

C57BL/6 mice were subcutaneously injected with 0.5×10⁶ B16F10 cells. After tumor inoculation (day 0), the mice were given three doses of doxetaxel chemotherapy (10 mg/kg) on days 1, 4, and 7 and single dose of TGFRt15-TGFRs (3 mg/kg) combined with monoclonal antibody targeting a tumor antigen anti-TYRP-1 antibody TA99 (200 μg) on day 8. Tumor-bearing mice treated with saline or doxetaxel chemotherapy (10 mg/kg) on days 1, 4, and 7 served as controls. Blood was drawn from submandibular vein on days 1, 3, 5, and 10 after immunotherapy treatment (day 8) in tubes containing with EDTA and immediately placed on ice. The blood was centrifuged for 15 minutes at 3,000 rpm at room temperature to separate plasma. Plasma samples were aliquoted and stored at −80° C. Aliquots were diluted 2-fold in PBS and analyzed using a Mouse Cytokine Array Proinflammatory Focused 10-plex (MDF10) assay.

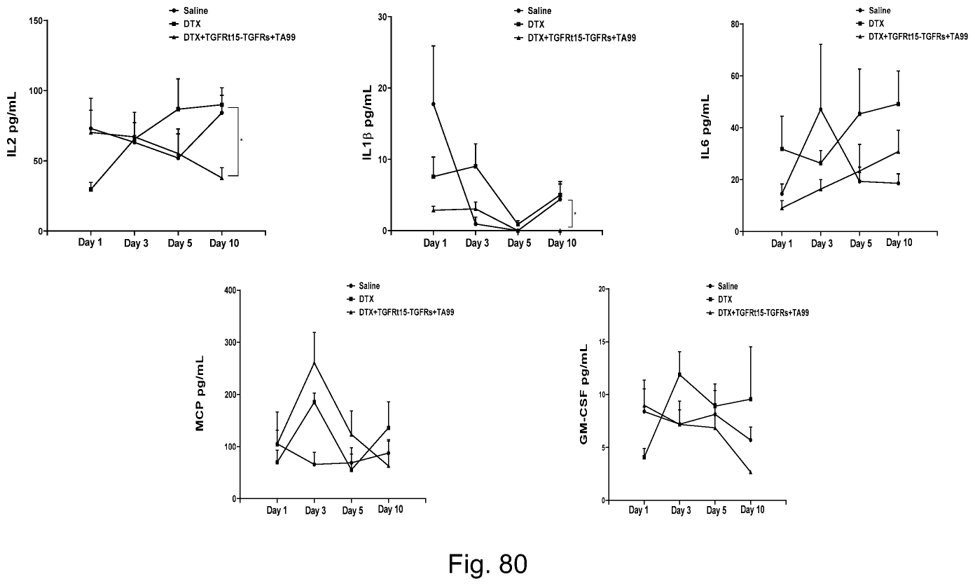

As shown in FIG. 80, the results show that administration of TGFRt15-TGFRs+TA99 reduced in plasma levels of IL2, IL-1β, IL6, MCP-1, and GM-CSF in tumor-bearing mice on day 10 post-treatment, when compared to the chemotherapy treatment group. This effect is consistent with the immunostimulatory activities of TGFRt15-TGFRs.

### Example 37: TGFRt15-TGFRs Treatment Enhances NK and CD8⁺ Expansion in the Spleen of B16F10 Tumor Bearing Mice

C57BL/6 mice were subcutaneously injected with 0.5×10⁶ B16F10 cells. After tumor inoculation (day 0), the mice were given three doses of doxetaxel chemotherapy (10 mg/kg) on days 1, 4, and 7 and single dose of TGFRt15-TGFRs (3 mg/kg) combined with monoclonal antibody targeting a tumor antigen anti-TYRP-1 antibody TA99 (200 μg) on day 8. Tumor-bearing mice treated with saline or doxetaxel chemotherapy (10 mg/kg) on days 1, 4, and 7 served as controls. Mice were sacrificed and the spleens were harvested at days 3, 5, and 10 post-immunotherapy (day 8). The spleens were crushed with flat back end of the sterile piston/plunger of 3 cc syringe to release the splenocytes. The splenocytes were passed through a 70-μM cell strainer and homogenized into a single cell suspension. The RBCs were lysed in ACK lysis buffer and the splenocytes were washed and stained with antibodies for cell-surface expression of NK and CD8 (BioLegend), for 30 minutes at RT. After two washes, the cells were resuspended in fixation buffer and analyzed by flow cytometry (Celesta-BD Bioscience).

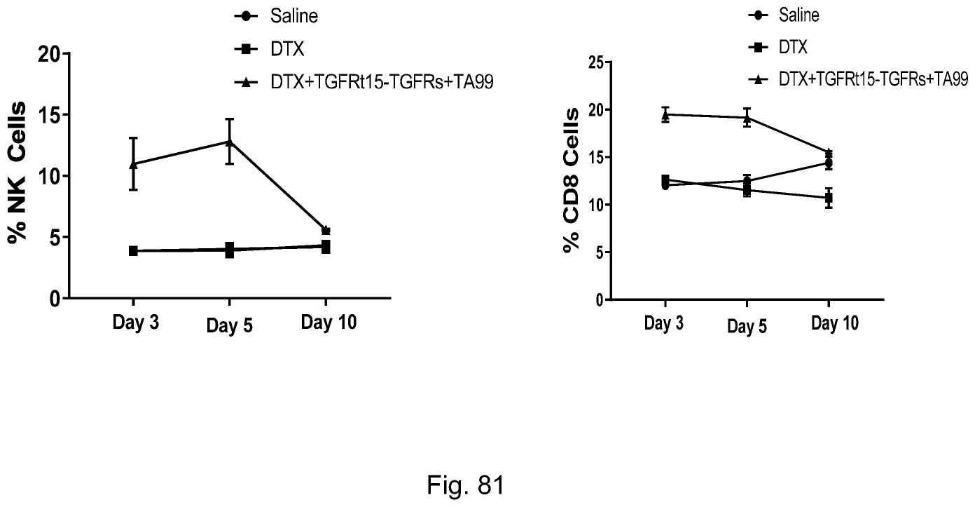

As shown in the FIG. 81, the expansion of NK and CD8+ cells were seen in the spleen at days 3 and 5 post-TGFRt15-TGFRs+TA99 therapy, when compared to the saline or chemotherapy treatment groups. Levels of NK cells (but not the CD8+ cells) were still found to be elevated at day 10 post-immunotherapy in the spleen of tumor-bearing mice, when compared levels in the spleens of the chemotherapy treatment group. These effects are consistent with the immunostimulatory activities of TGFRt15-TGFRs.

### Example 38: TGFRt15-TGFRs Treatment Enhances Glycolytic Activity of Splenocytes in B16F10 Tumor Bearing Mice

C57BL/6 mice were subcutaneously injected with 0.5×10⁶ B16F10 cells. After tumor inoculation (day 0), the mice were given three doses of doxetaxel chemotherapy (10 mg/kg) on days 1, 4, and 7 and single dose of TGFRt15-TGFRs (3 mg/kg) combined with monoclonal antibody targeting a tumor antigen anti-TYRP-1 antibody TA99 (200 μg) on day 8. Tumor-bearing mice treated with saline or doxetaxel chemotherapy (10 mg/kg) on days 1, 4, and 7 served as controls. Mice were sacrificed and the spleens were harvested at days 3, 5, and 10 post-immunotherapy (day 8). The spleens were crushed with flat back end of the sterile piston/plunger of 3 cc syringe to release the splenocytes. The splenocytes were passed through a 70-μM cell strainer and homogenized into a single cell suspension. The RBCs were lysed in ACK lysis buffer and the splenocytes were washed and counted. To measure the glycolytic activity of the splenocytes, the cells were washed and resuspended in seahorse media and resuspended in 4×10⁶ cells/mL. The cells were seeded at 50 μL/well in Cell-Tak-coated Seahorse Bioanalyzer XFe96 culture plates in Seahorse XF RPMI medium, pH 7.4 supplemented with 2 mM L-glutamine for glycolysis stress test. The cells were allowed to attach to the plate for 30 minutes at 37° C. Additionally, 130 μL of the assay medium was added to each well of the plate (also the background wells). The plate was incubated in 37° C., non-CO₂ incubator for 1 hr. For glycolysis stress test the calibration plate contained 10× solution of glucose/oligomycin/2DG prepared in Seahorse assay media and 20 μL of glucose/oligomycin/2DG were added to each of the ports of the extracellular flux plate that was calibrated overnight. The glycolysis stress test is based on extracellular acidification rate (ECAR) and measures three key parameters of glycolytic function including glycolysis, glycolytic capacity, and glycolytic reserve. Complete ECAR analysis consisted of four stages: non glycolytic acidification (without drugs), glycolysis (10 mM glucose), maximal glycolysis induction/glycolytic capacity (2 μM oligomycin), and glycolysis reserve (100 mM 2-DG). At the end of the experiment the data was exported as a Graph Pad Prism file. The XF glycolysis stress test report generator automatically calculated the XF cell glycolysis stress test parameters from the Wave data. The data was analyzed using the Wave software (Agilent).

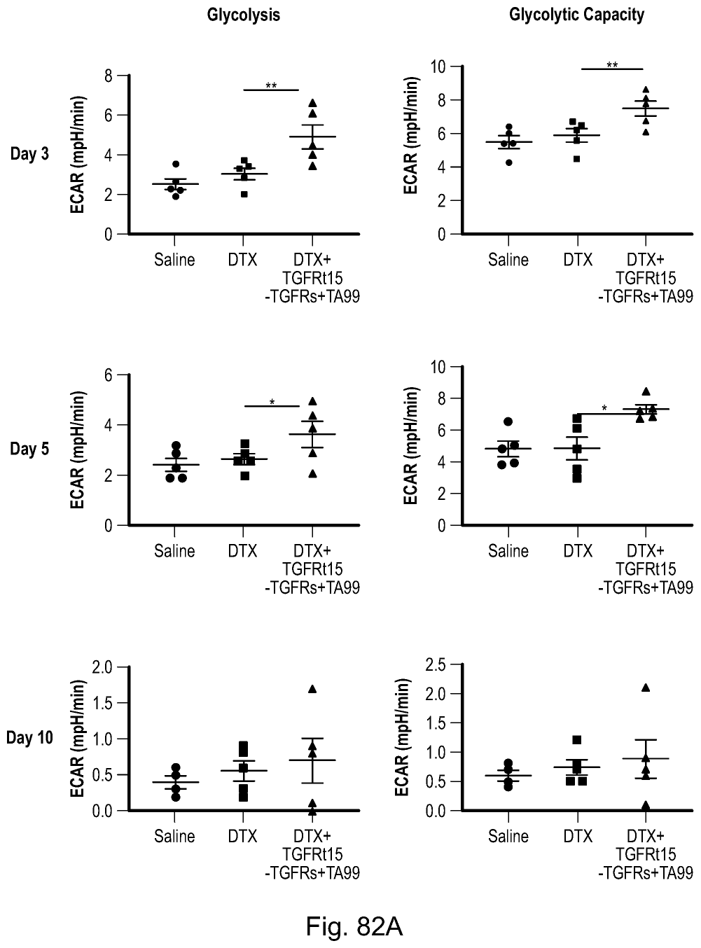

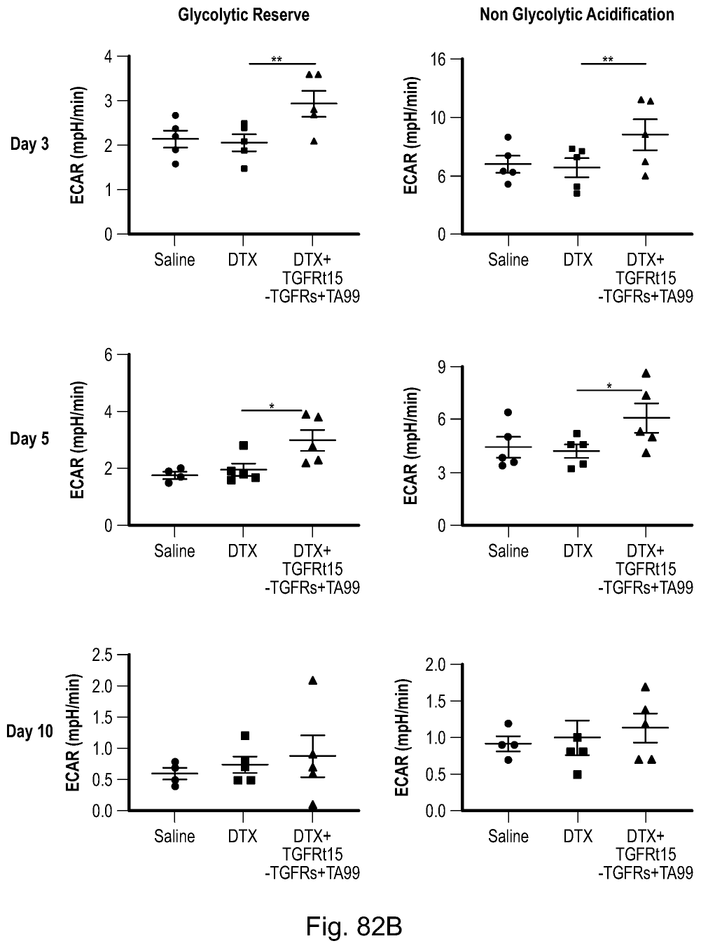

As shown in the FIGS. 82A and 82B, the splenocytes isolated from tumor-bearing mice at day 3 and day 5 after TGFRt15-TGFRs+TA99 therapy showed enhanced basal glycolysis, capacity and reserve rate, when compared to splenocytes of the saline or chemotherapy treatment groups. However no significant difference in the splenocyte glycolytic activity was observed at day 10 post-immunotherapy. These effects are consistent with the immunostimulatory activities of TGFRt15-TGFRs.

### Example 39: TGFRt15-TGFRs Treatment Enhances Mitochondrial Respiration of Splenocytes in B16F10 Tumor Bearing Mice

C57BL/6 mice were subcutaneously injected with 0.5×10⁶ B16F10 cells. After tumor inoculation (day 0), the mice were given three doses of doxetaxel chemotherapy (10 mg/kg) on days 1, 4, and 7 and single dose of TGFRt15-TGFRs (3 mg/kg) combined with monoclonal antibody targeting a tumor antigen anti-TYRP-1 antibody TA99 (200 μg) on day 8. Tumor-bearing mice treated with saline or doxetaxel chemotherapy (10 mg/kg) on days 1, 4, and 7 served as controls. Mice were sacrificed and the spleens were harvested at days 3, 5, and 10 post-immunotherapy (day 8). The spleens were crushed with flat back end of the sterile piston/plunger of 3 cc syringe to release the splenocytes. The splenocytes were passed through a 70 μM cell strainer and homogenized into a single cell suspension. The RBCs were lysed in ACK lysis buffer and the splenocytes were washed and counted. To measure the mitochondrial respiration of the splenocytes, the cells were washed and resuspended in seahorse media and resuspended in 4×10⁶ cells/mL. The cells were seeded at 50 L/well in Cell-Tak-coated Seahorse Bioanalyzer XFe96 culture plates in Seahorse XF RPMI medium, pH 7.4 supplemented with 2 mM L-glutamine for glycolysis stress test. For mitochondrial stress test, the cells were seeded in Seahorse XF RPMI medium, pH 7.4 supplemented with 10 mM glucose and 2 mM L-glutamine. The cells were allowed to attach to the plate for 30 minutes at 37° C. Additionally, 130 μL of the assay medium was added to each well of the plate (also the background wells). The plate was incubated in 37° C., non-CO₂ incubator for 1 hr. For mitochondrial stress test, the Calibration plate contained 10× solution of oligomycin/FCCP/rotenone prepared in Seahorse assay media and 20 μL of oligomycin, FCCP, and rotenone was added to each of the ports of the extracellular flux plate that was calibrated overnight. Oxygen Consumption Rate (OCR) was measured using an XFe96 Extracellular Flux Analyzer. Complete OCR analysis consisted of four stages: basal respiration (without drugs), ATP-linked respiration/Proton leak (1.5 μM mM Oligomycin), maximal respiration (2 μM FCCP), and spare respiration (0.5 μM Rotenone). At the end of the experiment, the data was exported as a Graph Pad Prism file. The XF mitochondrial stress test report generator automatically calculates the XF mitochondrial stress test parameters from the Wave data that have been exported to Excel. The data was analyzed by using the Wave software (Agilent).

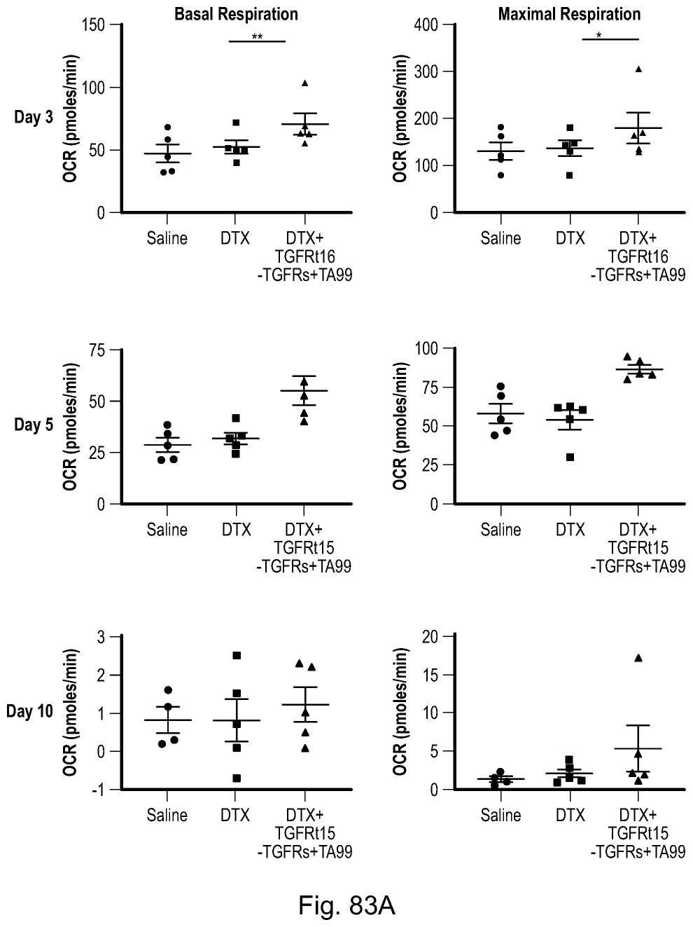

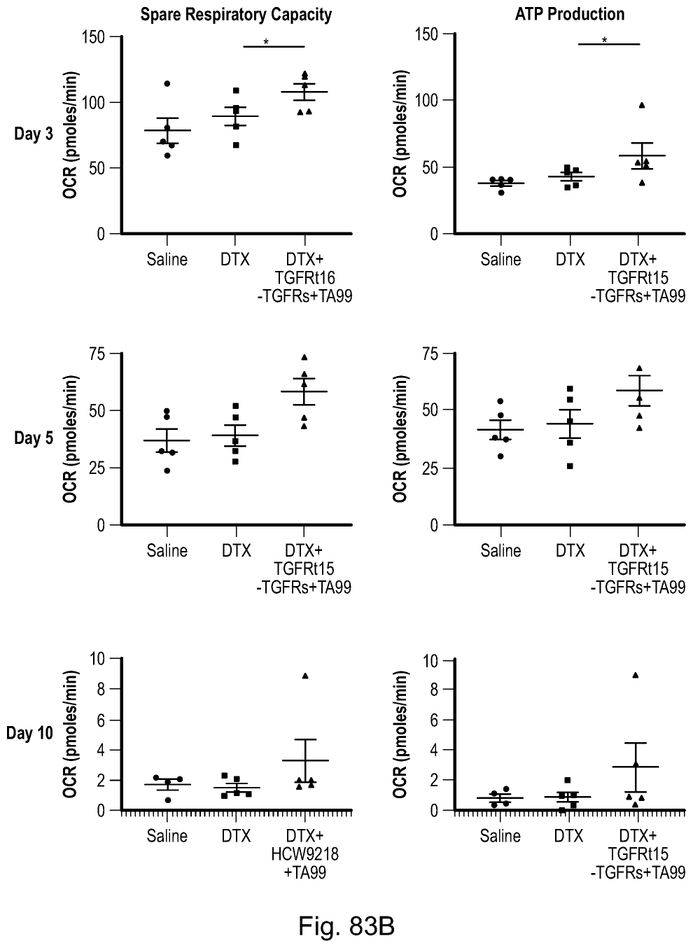

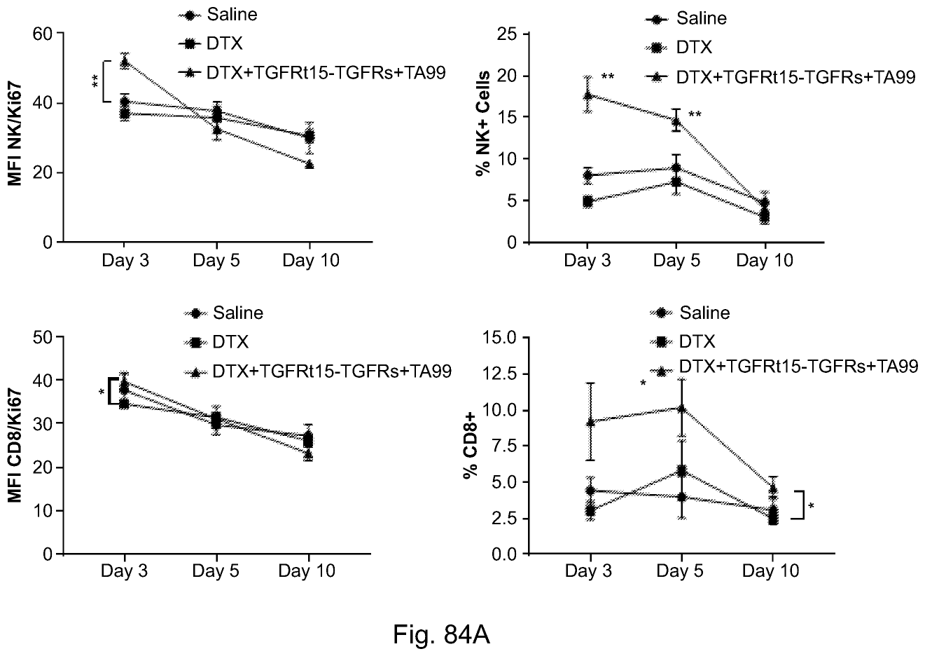

As shown in the FIGS. 83A and 83B, the splenocytes isolated from tumor-bearing mice at day 3 and day 5 after TGFRt15-TGFRs+TA99 therapy showed enhanced basal respiration, mitochondria respiration, capacity and ATP production, when compared to splenocytes of the saline or chemotherapy treatment groups. However no significant difference in the splenocyte mitochondrial respiration was observed at day 10 post-immunotherapy. These effects are consistent with the immunostimulatory activities of TGFRt15-TGFRs. Metabolic pathways like oxidative metabolism and glycolysis are known to preferentially fuel the cell fate decisions and effector functions of immune cells. Therefore, TGFRt15-TGFRs mediated increased glycolytic activity and mitochondrial respiration might be associated with the activation of NK and CD8+ immune cells in the blood, spleen, and tumor of the mice.

### Example 40: TGFRt15-TGFRs Treatment Enhances NK and CD8 Immune Cell Infiltration (TILs) into Tumors of B16F10 Tumor Bearing Mice

C57BL/6 mice were subcutaneously injected with 0.5×10⁶ B16F10 cells. After tumor inoculation (day 0), the mice were given three doses of doxetaxel chemotherapy (10 mg/kg) on days 1, 4, and 7 and single dose of TGFRt15-TGFRs (3 mg/kg) combined with monoclonal antibody targeting a tumor antigen anti-TYRP-1 antibody TA99 (200 μg) on day 8. Tumor-bearing mice treated with saline or doxetaxel chemotherapy (10 mg/kg) on days 1, 4, and 7 served as controls. Mice were sacrificed and the tumors were harvested at days 3, 5, and 10 post-immunotherapy. The tumor tissue was dissociated into single cell suspension by collagenase digestion to determine the tumor-infiltrating immune cells. The single cell suspension was layered on Ficoll-Paque media followed by density gradient centrifugation to separate the lymphocytes and tumor cells. The cells were centrifuged at 1000 g for 20 minutes at 20° C. with slow acceleration and break turned off. After centrifugation the Ficoll-Paque results in a distinct separation between two layers. The TILs are found on the interface between the media and Ficoll-Paque, while the pellet consists of the tumor cells. The TILs were carefully removed from the interface and washed with complete RPMI media. After washing, the RBCs were lysed in ACK buffer for 5 minutes at room temperature. The cells were washed in FACS buffer (1×PBS (Hyclone) with 0.5% BSA (EMD Millipore) and 0.001% Sodium Azide (Sigma)). To assess the different types of immune cells in tumor, the cells were stained with antibodies for cell-surface CD8, NK1.1, CD25, and GzB (BioLegend) for 30 minutes at RT. After surface staining, the remaining cells were washed (1500 RPM for 5 minutes at room temperature) in FACS buffer (1×PBS (Hyclone) with 0.5% BSA (EMD Millipore) and 0.001% Sodium Azide (Sigma)). After two washes, the cells were resuspended in fixation buffer. After fixation cells were washed and treated with permeabilization buffer (Invitrogen) for 20 minutes at 4° C. followed by wash with permeabilization buffer (Invitrogen). The cells were then stained for intracellular markers for proliferation (Ki67) for 30 minutes at RT. After two washes, the cells were resuspended in fixation buffer and analyzed by flow cytometry (Celesta-BD Bioscience).

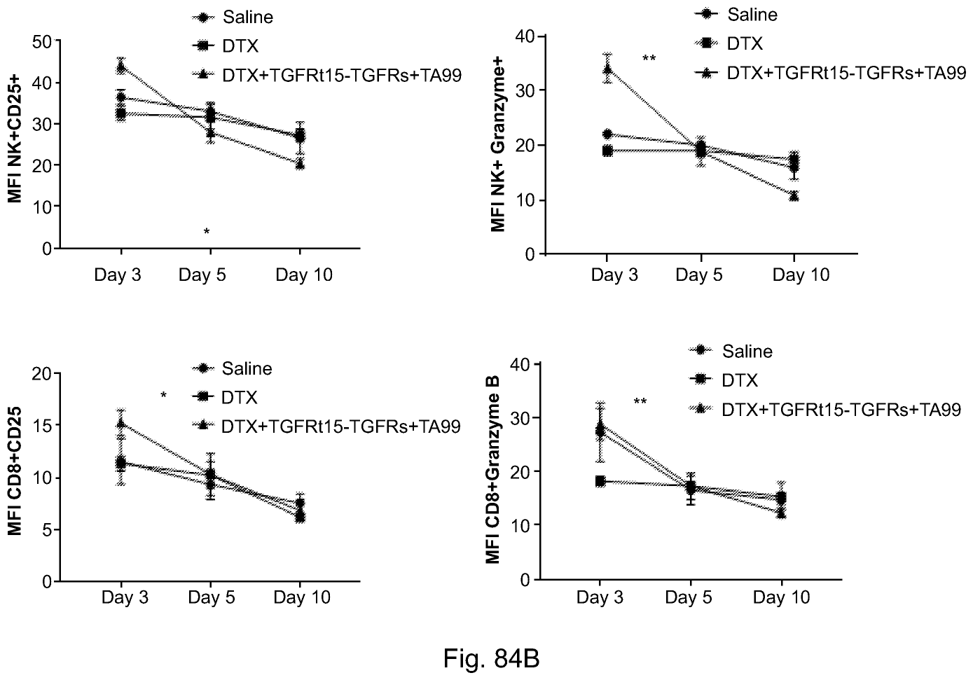

As shown in FIGS. 84A and 84B, tumor analysis showed high levels of Ki67-positive NK and CD8 cells at day 3 post-therapy. Expansion of NK and CD8+ cells (based on % of lymphocytes in tumors) was found at day 3 and day 5 post-TGFRt15-TGFRs+TA99 therapy, when compared to the chemotherapy treatment group. Tumors CD8+ cells were elevated even at day 10 post-immunotherapy. Both NK and CD8+ showed the expression of activation markers CD25 and granzyme B at day 3 post-TGFRt15-TGFRs+TA99 therapy, when compared to immune cells of the chemotherapy treatment group. These effects are consistent with the immunostimulatory activities of TGFRt15-TGFRs and are comparable to changes seen in the blood and splenocytes of tumor-bearing mice.

### Example 41: Histopathological Analysis of Tumors Following TGFRt15-TGFRs Treatment

C57BL/6 mice were subcutaneously injected with 0.5×10⁶ B16F10 cells. After tumor inoculation (day 0), the mice were given three doses of doxetaxel chemotherapy (10 mg/kg) on days 1, 4, and 7 and single dose of TGFRt15-TGFRs (3 mg/kg) combined with monoclonal antibody targeting a tumor antigen anti-TYRP-1 antibody TA99 (200 μg) on day 8. Tumor-bearing mice treated with saline or doxetaxel chemotherapy (10 mg/kg) on days 1, 4, and 7 served as controls. Blood was drawn from submandibular vein on days 1, 3, 5, and 10 after immunotherapy treatment (day 8). On day 10 post-immunotherapy, the mice were sacrificed, and tumors were isolated. For the histological analysis, tumor samples were fixed in 10% formalin solution and were embedded in paraffin and cut at 5 μm. The sections were stained with H & E to assess tissue and cellular morphology. The slides were scored based on the mitotic and necrotic activity of the tumor. The percentage necrosis in the tumor was scored as, +1 (0-20%), +2 (20-40%), and +3 (40-60%). The Mitotic Index of the tumor was scored as +1=Moderate (1-5 per high power field) and +2=Extensive (>5 per high power field).

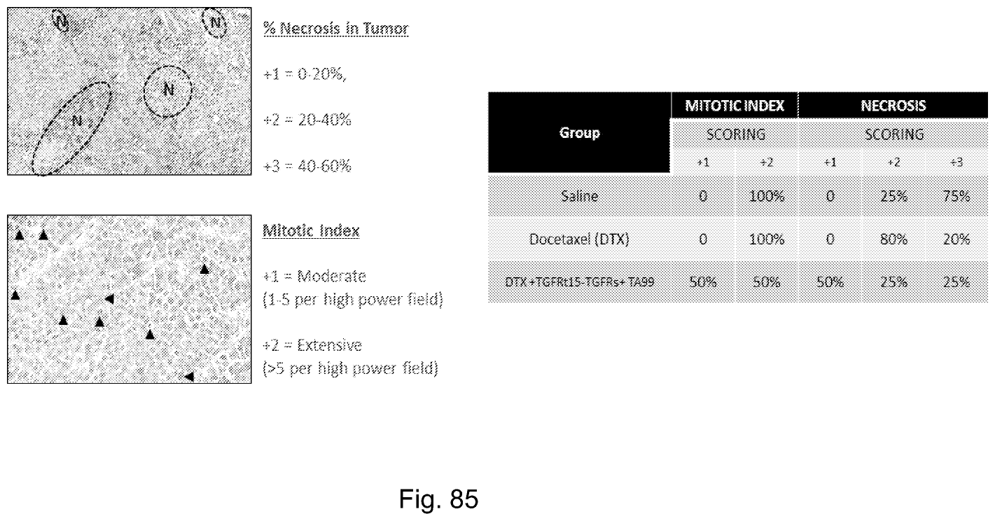

As shown in FIG. 85, following TGFRt15-TGFRs+TA99 treatment, tumors displayed less mitotic and necrotic activity. The mitotic index is correlated to the dividing cells and presence of necrosis is a measure of more aggressive features and poor prognosis. Hence TGFRt15-TGFRs is a promising therapy in pre-clinical murine models for testing of combination tumor immunotherapy.

### Example 42: Anti-PD-L1 Antibody in Combination with TGFRt15-TGFRs+TA99 and Chemotherapy in B16F10 Melanoma Mouse Model

C57BL/6 mice were subcutaneously injected with 0.5×10⁶ B16F10 cells. After tumor inoculation (day 0), the mice were given three doses of doxetaxel chemotherapy (10 mg/kg) on days 1, 4, and 7. Tumor-bearing mice treated with only saline or doxetaxel chemotherapy (10 mg/kg) on days 1, 4, and 7 served as controls. The remaining mice were randomized in two groups, one group was treated with anti-mPD-L1 antibody (2×10 mg/kg) and the other group was treated with TGFRt15-TGFRs (3 mg/kg) with TA99 (200 μg) on day 8. After 6 days, the mice which received the TGFRt15-TGFRs with TA99 were given anti-mPD-L1 antibody (2×10 mg/kg) and mice which received anti-mPD-L1 antibody were treated with TGFRt15-TGFRs (3 mg/kg) with TA99 (200 μg). The anti-mPD-L1 antibody was given as two doses on days 8 and 10 or days 14 and 16. Tumor growth was monitored by caliper measurement, and tumor volume was calculated using the formula V=(L×W2)/2, where L is the largest tumor diameter and W is the perpendicular tumor diameter. N=6-8 mice/group.

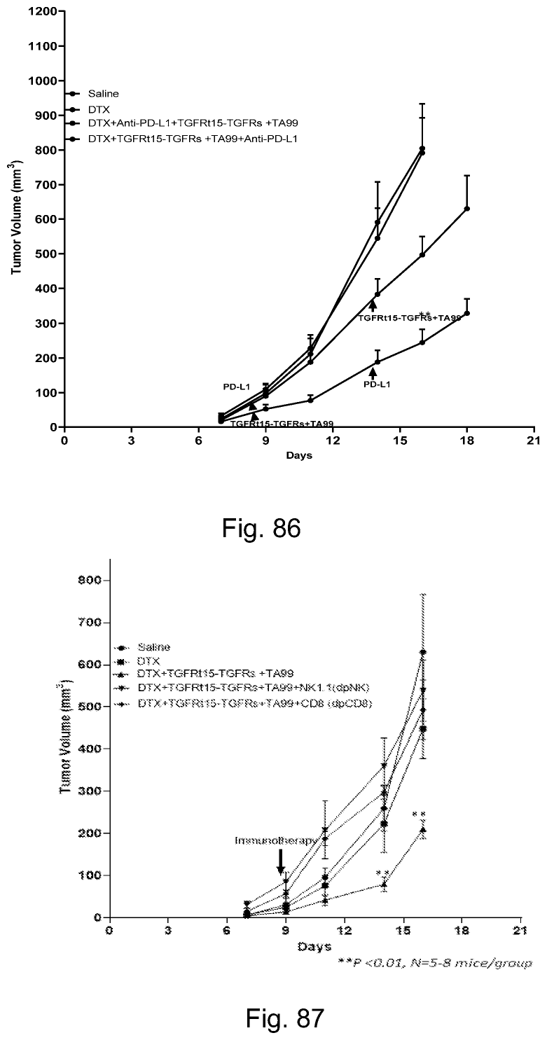

As shown in the FIG. 86, TGFRt15-TGFRs+TA99 administration following by anti-PD-L1 antibody treatment resulted in better antitumor activity in B16F10 tumor-bearing mice as compared to treatment with anti-PD-L1 antibody and then TGFRt15-TGFRs+TA99. Therefore, combining TGFRt15-TGFRs with anti-PD-L1 antibody may be advantageous in treating tumors that are resistance to anti-PD-L1 antibody therapy.

### Example 43: Anti-Tumor Efficacy of TGFRt15-TGFRs in B16F10 Melanoma Mouse Model is Dependent on NK and CD8⁺ T Cells

Groups of C57BL/6 mice (N=6-8 mice/group) were treated with three doses of NK1.1 Ab (500 μg) or CD8⁺a (500 μg) antibody intraperitoneal every third day to deplete the NK and CD8 cells. Blood was drawn and analyzed for NK and CD8⁺ lymphocyte levels before the B16F10 tumor implantation. Untreated mice served as immunocompetent controls. C57BL/6 mice were subcutaneously injected with 0.5×10⁶ B16F10 cells. After tumor inoculation (day 0), the mice were given three doses of docetaxel (10 mg/kg) on days 1, 4, and 7, followed by single dose of TGFRt15-TGFRs (3 mg/kg)+TA99 (200 μg) on day 8. Tumor growth was monitored by caliper measurement, and tumor volume was calculated using the formula V=(L×W2)/2, where L is the largest tumor diameter and W is the perpendicular tumor diameter.

As shown in FIG. 87, B16F10 tumor bearing mice treated with TGFRt15-TGFRs in combination with TA99 and chemotherapy showed a significant reduction in B16F10 tumor volume, when compared to tumors of the saline or chemotherapy treatment groups. However, when the mice were depleted for NK and CD8+ cell subsets, there was no effect of immunotherapy on the anti-antitumor activity. This experiment shows that both the NK and CD8+ immune cells play an important role in TGFRt15-TGFRs mediated anti-tumor activity.

### Example 44: Comparison of TGFRt15-TGFRs and TGFRt15*-TGFRs Treatment in Reducing Senescence Markers in Liver and Lung Tissues of B16F10 Tumor-Bearing Mice Following Chemotherapy

C57BL/6, 6-8-week-old mice were purchased from the Jackson Laboratory. Mice were housed in a temperature and light controlled environment. Mice were divided into five groups as follows: saline control group (n=7), docetaxel (DTX) group (n=7), DTX+TGFRt15-TGFRs group (n=7), DTX+TGFRt15*-TGFRs group (n=7), and DTX+IL15SA group (n=7). B16F10 tumor cells (1×10⁷ cells/mouse) were implanted in mice on day 0. The mice were treated subcutaneously with 10 mg/kg docetaxel on days 1, 4, and 7. On day 8, the mice were treated subcutaneously with PBS, TGFRt15-TGFRs (3 mg/kg), TGFRt15*-TGFRs (3 mg/kg), or IL15SA (0.5 mg/kg). The mice were euthanized day 17 post-treatment and liver and lungs were harvested in order to evaluate the gene expression of senescence markers p21, IL-1α, and IL6 for liver and p21 and IL-1α for lung by quantitative-PCR in tissues after treatment with TGFRt15-TGFRs or TGFRt15*-TGFRs and control groups. Harvested organs were stored in liquid nitrogen in 1.7 mL Eppendorf tubes. The samples were homogenized by using homogenizer in 1 mL of Trizol (Thermo Fischer). Homogenized tissues were transferred in fresh Eppendorf tubes. Total RNA was extracted using RNeasy Mini Kit (Qiagen #74106) according to the manufacturer's instructions. One μg of total RNA was used for cDNA synthesis using the QuantiTect Reverse Transcription Kit (Qiagen). Real-time PCR was carried out with CFX96 Detection System (Bio-Rad) using FAM-labeled predesigned primers purchased from Thermo Scientific. Reactions were run in triplicate for all the genes examined. The housekeeping gene 18S ribosomal RNA was used as an internal control to normalize the variability in expression levels. The expression of each target mRNA relative to 18S rRNA was calculated based on Ct as 2^{-Δ(Δct)}, in which ΔCt=Ct target−Ct18S.

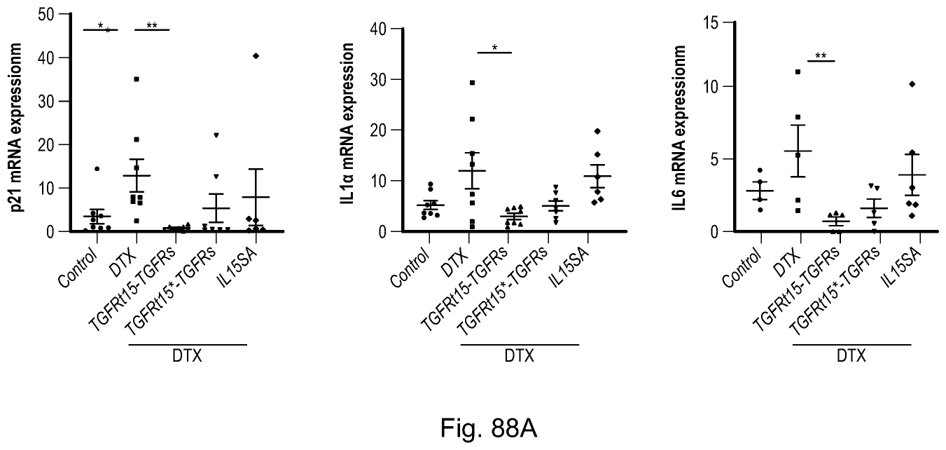

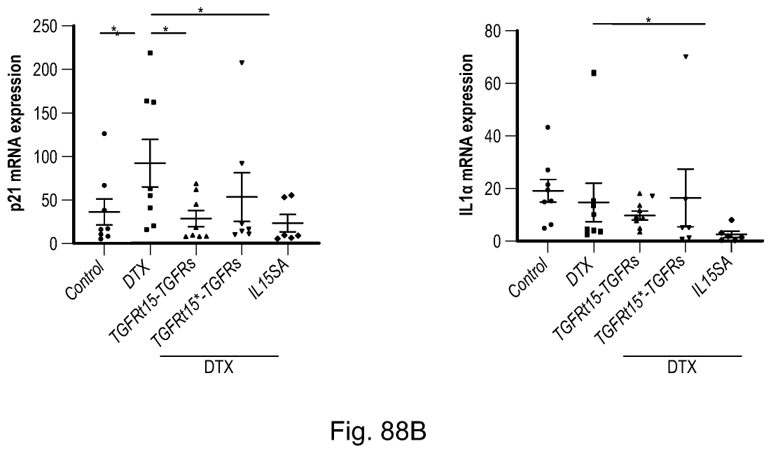

As shown in FIG. 88, the senescence markers p21, IL-1α, and IL6 showed decreased gene expression in liver (A) and lung (B) tissues in both TGFRt15-TGFRs and TGFRt15*-TGFRs-treated tumor bearing mice, when compared to gene expression in tissues of chemotherapy treated mice.

### Example 45: TGFRt15-TGFRs Treatment in Reducing Chemotherapy-Induced Senescent Tumor Cells In Vivo

B16F10 melanoma cells were stably transduced with GFP lentiviral plasmid and the GFP-expressing tumor cells (B16F10-GFP) were selected by growth in puromycin containing media. Almost 95% B16F10 melanoma cells were GFP-positive as analyzed by FACS. To induce senescence, B16F10-GFP cells were treated with 7.5 μM docetaxel (DTX) for 3 days followed by 4 days recovery in the normal growth media. To quantify gene expression of senescence markers and NK cell ligands, docetaxel-treated B16F10 GFP cells (B16F10-GFP-SNC) were homogenized by using homogenizer in 1 mL of Trizol (Thermo Fischer). Homogenized cells were transferred in fresh Eppendorf tubes. Total RNA was extracted using RNeasy Mini Kit (Qiagen #74106) according to the manufacturer's instructions. One μg of total RNA was used for cDNA synthesis using the QuantiTect Reverse Transcription Kit (Qiagen). Real-time PCR was carried out with CFX96 Detection System (Bio-Rad) using FAM-labeled predesigned primers purchased from Thermo Scientific. The reactions were run in triplicate for all the genes examined. The housekeeping gene 18S ribosomal RNA was used as an internal control to normalize the variability in expression levels. The expression of each target mRNA relative to 18S rRNA was calculated based on Ct as 2^{-Δ(ΔCt)}, in which ΔCt=Ct target−Ct18S. The expression of different genes is plotted as fold-change in B16F10-GFP-SNC cells as compared to untreated B16F10-GFP cells.

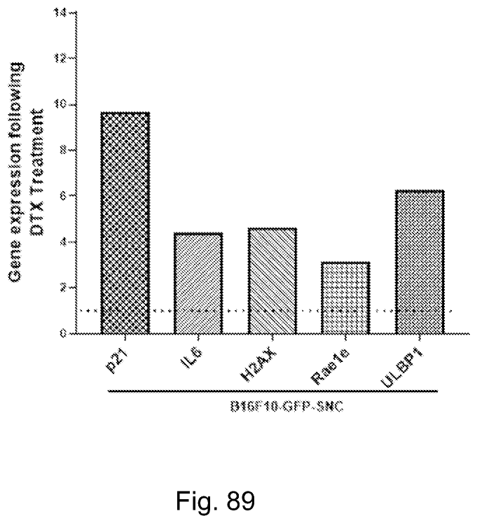

As shown in FIG. 89, real time PCR analysis showed that B16F10-GFP cells treated in vitro with docetaxel upregulated gene expression of senescence markers, p21, H2AX, and IL6, and NK cell ligands, Rae-1e and ULBP-1, when compared to untreated B16F10-GFP cells.

To determine whether chemotherapy-induced senescence tumor cells are reduced by immunotherapy in vivo, B16F10 parental melanoma cells (0.75×10⁶) were mixed with B16F10-GFP-SNC cells (0.75×10⁶) and injected the cell mixture subcutaneously in C57BL/6 mice. Mice were also injected with B16F10 and B16F10-GFP cells as controls. The B16F10 parent cells will grow to form tumor and B16F10-GFP-SNC cells will be the part of the tumor microenvironment. When tumors reached to approximately 350 mm³, mice bearing the mixed tumors were divided into 2 groups. One group received PBS as control and the other group received TGFRt15-TGFRs (3 mg/kg) with TA99 (200 μg) subcutaneously. The mice were sacrificed day 4 post-immunotherapy treatment. The tumor tissue was dissociated into single cell suspension by collagenase digestion to determine the tumor-infiltrating immune cells. The single cell suspension was layered on Ficoll-Paque media followed by density gradient centrifugation to separate the lymphocytes and tumor cells. The cells were centrifuged at 1000 g for 20 minutes at 20° C. with slow acceleration and break turned off. After centrifugation the Ficoll-Paque results in a distinct separation between two layers. The TILs are found on the interface between the media and Ficoll-Paque, while the pellet consists of the tumor cells. The TILs were carefully removed from the interface and washed with complete RPMI media. After washing, the RBCs were lysed in ACK buffer for 5 minutes at room temperature. The remaining cells were washed in FACS buffer (1×PBS (Hyclone) with 0.5% BSA (EMD Millipore) and 0.001% Sodium Azide (Sigma)). To assess the different types of immune cells in tumor, the cells were stained with antibodies specific to cell-surface CD3, CD45, CD8, and NK1.1 (BioLegend) for 30 minutes at RT. After surface staining, cells were washed (1500 RPM for 5 minutes at room temperature) in FACS buffer (1×PBS (Hyclone) with 0.5% BSA (EMD Millipore) and 0.001% Sodium Azide (Sigma)). After two washes, the cells were resuspended in fixation buffer. After fixation, the cells were washed and treated with permeabilization buffer (Invitrogen) for 20 minutes at 4° C. followed by wash with permeabilization buffer (Invitrogen). The cells were then stained for intracellular markers (Ki67) for proliferation for 30 minutes at RT. After two washes, the cells were resuspended in fixation buffer and analyzed by flow cytometry (Celesta-BD Bioscience).

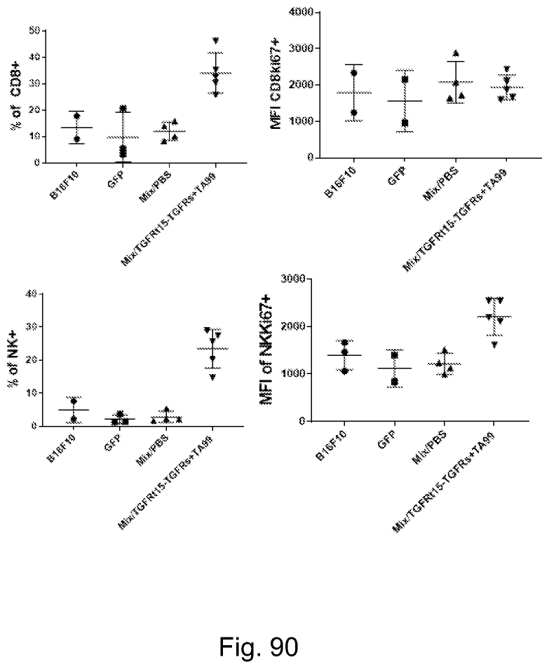

As shown in FIG. 90, the percentage of CD8⁺ T cells and natural killer (NK) cells were increased after 4 days post-treatment in the tumor following TGFRt15-TGFRs+TA99 treatment, compared to controls. These results demonstrate that TGFRt15-TGFRs is able to stimulate infiltration of CD8⁺ T cells and NK cells in the tumor. Both CD8⁺ T cells and NK immune cells were also able to proliferate in the tumor as measured by the Ki67 marker.

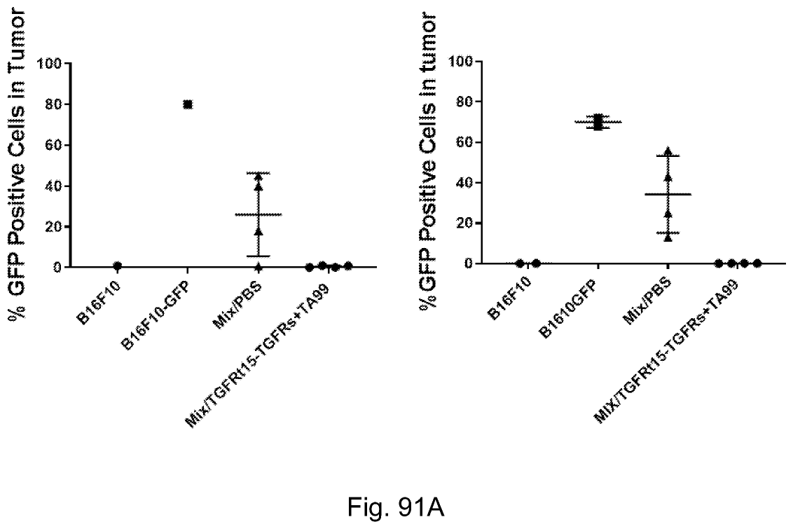

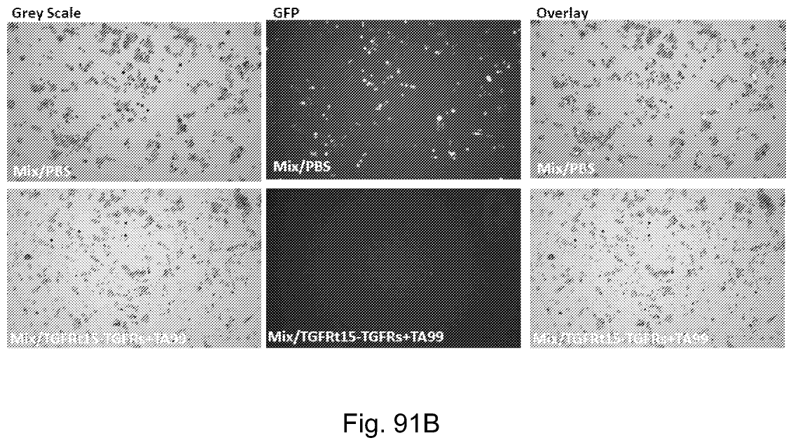

To determine whether chemotherapy-induced senescence tumor cells are reduced by immunotherapy in vivo, B16F10 parental melanoma cells (0.75×10⁶) were mixed with B16F10-GFP-SNC cells (0.75×10⁶) and injected the cell mixture subcutaneously in C57BL/6 mice. Mice were also injected with B16F10 and B16F10-GFP cells as controls. The B16F10 parent cells will grow to form tumor and B16F10-GFP-SNC cells will be the part of the tumor microenvironment. When tumors reached to approximately 350 mm³, mice bearing the mixed tumors were divided into 2 groups. One group received PBS as control and the other group received TGFRt15-TGFRs (3 mg/kg) with TA99 (200 μg) subcutaneously. The mice were sacrificed after day 4 and day 10 post-immunotherapy treatment. The tumor tissue was dissociated into single cell suspension by collagenase digestion to determine the tumor-infiltrating immune cells and GFP-positive cells in the tumor. Flow cytometry analysis (FIG. 91A) on tumor cells showed that mice which received immunotherapy treatment showed lower number of GFP-positive cells 4 days and 10 days post-treatment as compared to the PBS control group. Tumor cells were plated in a 24-well plate to evaluate by fluorescence microscopy (FIG. 91B).

Microscopic images also showed fewer GFP-positive cells in the tumor of immunotherapy-treated mice as compared to the control PBS-treated group. The GFP expression in the tumor is associated with the chemotherapy-induced B16F10-GFP senescence cells, therefore reduction in the GFP expression after immunotherapy treatment shows the successful elimination of senescence tumor cells in the tumor bearing mice.

### Example 46: TGFβ Levels in Kidney after Inducing Kidney Injury by Cisplatin and Treatment with TGFRt15-TGFRs by Tissue ELISA

The mouse kidney was harvested in order to evaluate changes in protein levels of the senescence markers TGFβ after inducing kidney injury by cisplatin and treatment with TGFRt15-TGFRs. C57BL/6, 8-week-old mice were purchased from the Jackson Laboratory. The mice were housed in a temperature and light controlled environment. The mice were injected with cisplatin (5 mg/kg, intraperitoneal) weekly for 3 weeks to induce kidney injury. One week after cisplatin, the mice were treated with either PBS or TGFRt15-TGFRs (3 mg/kg) (n=8/group). The mice were euthanized after 30 days of immunotherapy treatment and kidney were harvested and stored in liquid nitrogen in 1.7 mL-Eppendorf tubes. The samples were homogenized by using homogenizer in 0.3 mL of extraction buffer (Abcam). Homogenized tissues were transferred in fresh Eppendorf tubes. Protein levels in homogenized tissue were quantified using BCA Protein Assay Kit (Pierce). Mouse TGFβ ELISA (R&D System) was performed in 200 μg of tissue. The concentration of TGFβ was calculated in per milligram of tissue.

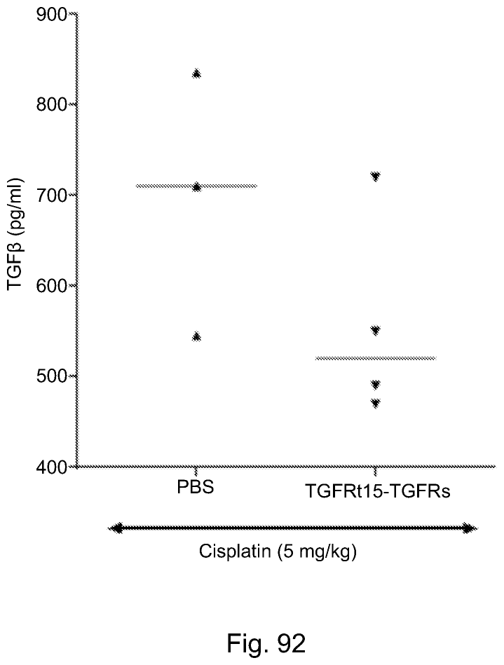

As shown in FIG. 92, the TGFβ level decreased in TGFRt15-TGFRs treated mice kidney compared to PBS control group. These results indicate that TGFRt15-TGFRs treatment is capable of provide long lasting activity in reducing TGFβ levels in tissues of chemotherapy-treated mice.

### Example 47: Toxicity of Subcutaneous Administration of TGFRt15-TGFRs in Mice

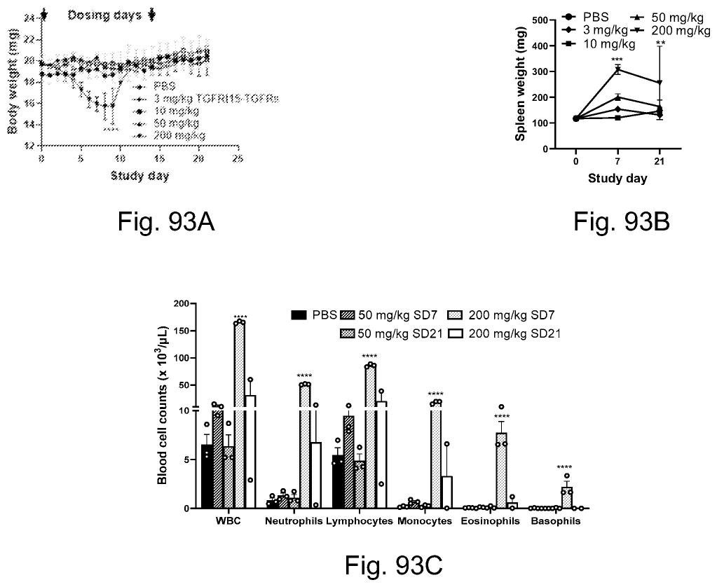

To further assess the dose-dependent toxicological effects of TGFRt15-TGFRs, female C57BL/6 mice (N=3/group) were administered one or two (every two weeks) subcutaneous doses of PBS or TGFRt15-TGFRs at 3, 10, 50, and 200 mg/kg. Animals were monitored for signs of study drug-related toxicities, changes in body weight during the study period and hematology and serum chemistry parameters at day 7 post-dosing. Mice receiving 200 mg/kg TGFRt15-TGFRs exhibited significant body weight loss beginning 4 days after the first injection (study day (SD) 0) and reaching a nadir between SD6-9, before returning to pre-dose levels by SD11 (FIG. 93A). Mortality was observed in one mouse of the 200 mg/kg group on SD9. There were no apparent treatment-mediated effects on body weight or other clinical signs in any other dose group or after the second TGFRt15-TGFRs dose at 200 mg/kg. Spleen weights increased in a dose dependent manner following one or two doses of TGFRt15-TGFRs (FIG. 93B). Compared to the PBS group, mice also exhibited a 25-fold increase in WBC counts 7 days after a single 200 mg/kg dose of TGFRt15-TGFRs, which remained 5-fold higher 7 days after the second 200 mg/kg dose (FIG. 93C, Tables 3 and 4). WBC subset analysis showed a 16-fold increase in absolute lymphocyte counts and >50-fold increase in neutrophil, monocyte, eosinophil, and basophil counts at SD7 in the 200 mg/kg group. These changes were not observed at lower TGFRt15-TGFRs dose levels but were similar to those reported for C57BL/6 mice treated subcutaneously treatment with IL-15/IL-15Rα complexes (Liu et al., Cytokine 107:105-112, 2018). Other hematology and serum chemistry parameters were similar in the TGFRt15-TGFRs and PBS treated animals and were generally within expected ranges for C57BL/6 mice (Tables 3 and 4). TGFRt15-TGFRs-mediated effects were greatest 7 days after the first dose and were reduced after the second dose, consistent with previous studies showing decreased immune responses in mice following repeat dosing with IL-15/IL-15Rα (Elpek et al., PNAS 107:21647-21652, 2010; Frutoso et al., J Immunol 201:493-506, 2018). Overall, TGFRt15-TGFRs was well tolerated by C57BL/6 mice at dose levels up to of 50 mg/kg.

TABLE 3
Hematology and serum chemistry parameters of C57BL/6 mice on Study Day 7 after single dose of TGFRt15-TGFRs.

| | Study Day 7 | | | | | | | | | | | | | | |
|---|---|---|---|---|---|---|---|---|---|---|---|---|---|---|---|
| | | | | TGFRt15-TGFRs | | | | | | | | | | | |
| | PBS | | | 3 mg/kg | | | 10 mg/kg | | | 50 mg/kg | | | 200 mg/kg | | |
| Parameters | Mean | SD | N | Mean | SD | N | Mean | SD | N | Mean | SD | N | Mean | SD | N |
| WBC count (×10³/μL) | 6.53 | 1.80 | 3 | 6.63 | 1.37 | 3 | 5.07 | 1.53 | 3 | 11.57 | 2.99 | 3 | 165.37 | 2.20 | 3 |
| RBC count (×10⁶/μL) | 7.59 | 0.90 | 3 | 6.44 | 0.34 | 3 | 7.03 | 0.34 | 3 | 6.56 | 0.68 | 3 | 6.25 | 0.84 | 3 |
| Hemoglobin (g/dL) | 10.1 | 0.8 | 3 | 9.3 | 0.0 | 3 | 9.6 | 0.3 | 3 | 8.7 | 1.1 | 3 | 9.4 | 1.2 | 3 |
| Hematocrit (%) | 36.0 | 3.2 | 3 | 31.8 | 2.3 | 3 | 33.0 | 1.9 | 3 | 30.8 | 3.3 | 3 | 29.9 | 4.0 | 3 |
| MCV(fL) | 47.3 | 1.5 | 3 | 49.3 | 1.5 | 3 | 46.7 | 0.6 | 3 | 47.0 | 0.0 | 3 | 48.0 | 0.0 | 3 |
| MCH (pg) | 13.3 | 0.6 | 3 | 14.3 | 0.6 | 3 | 13.7 | 0.6 | 3 | 13.3 | 0.6 | 3 | 15.0 | 1.0 | 3 |
| MCHC (%) | 28.0 | 0.0 | 3 | 29.7 | 2.1 | 3 | 29.0 | 1.0 | 3 | 28.3 | 1.5 | 3 | 31.3 | 1.5 | 3 |
| Neutrophils (×10³/μL) | 0.82 | 0.42 | 3 | 0.91 | 0.28 | 3 | 0.53 | 0.11 | 3 | 1.32 | 0.43 | 3 | 51.25 | 0.97 | 3 |
| Lymphocytes (×10³/μL) | 5.46 | 1.31 | 3 | 5.39 | 0.9 | 3 | 4.26 | 1.34 | 3 | 9.47 | 2.34 | 3 | 86.01 | 2.80 | 3 |
| Monocytes (×10³/μL) | 0.18 | 0.08 | 3 | 0.24 | 0.2 | 3 | 0.24 | 0.07 | 3 | 0.69 | 0.20 | 3 | 18.17 | 2.68 | 3 |
| Eosinophils (×10³/μL) | 0.07 | 0.02 | 3 | 0.06 | 0.02 | 3 | 0.05 | 0.02 | 3 | 0.08 | 0.08 | 3 | 7.73 | 2.02 | 3 |
| Basophils (×10³/μL) | 0.02 | 0.03 | 3 | 0.03 | 0.05 | 3 | 0.00 | 0.00 | 3 | 0.00 | 0.00 | 3 | 2.21 | 0.99 | 3 |
| Platelet count (×10³/μL) | 558.3 | 81.1 | 3 | 692.3 | 55.8 | 3 | 886.0 | 53.6 | 3 | 1004.3 | 60.2 | 3 | 467.3 | 32.5 | 3 |
| % Neutrophils | 12.0 | 3.0 | 3 | 13.7 | 3.1 | 3 | 10.7 | 1.2 | 3 | 11.3 | 1.2 | 3 | 31.0 | 1.0 | 3 |
| % Lymphocytes | 84.0 | 3.0 | 3 | 81.7 | 3.8 | 3 | 83.7 | 1.5 | 3 | 82.0 | 1.0 | 3 | 52.0 | 1.0 | 3 |
| % Monocytes | 2.67 | 0.58 | 3 | 3.33 | 2.31 | 3 | 4.67 | 0.58 | 3 | 6.00 | 1.00 | 3 | 11.00 | 1.73 | 3 |
| % Eosinophils | 1.00 | 0.00 | 3 | 1.00 | 0.00 | 3 | 1.00 | 0.00 | 3 | 0.67 | 0.58 | 3 | 4.67 | 1.15 | 3 |
| % Basophils | 0.33 | 0.58 | 3 | 0.33 | 0.58 | 3 | 0.00 | 0.00 | 3 | 0.00 | 0.00 | 3 | 1.33 | 0.58 | 3 |
| AST (U/L) | 84.3 | 28.2 | 3 | 69.0 | 9.2 | 3 | 137.7 | 108.6 | 3 | 71.7 | 2.5 | 3 | 162.3 | 11.8 | 3 |
| ALT (U/L) | 41.3 | 10.0 | 3 | 47.3 | 1.5 | 3 | 38.3 | 5.5 | 3 | 56.3 | 11.2 | 3 | 121.0 | 52.8 | 3 |
| Alkaline Phos. (U/L) | 113.7 | 17.0 | 3 | 112.0 | 8.7 | 3 | 248.3 | 218.8 | 3 | 95.0 | 7.8 | 3 | 83.0 | 16.6 | 3 |
| Total Bilirubin (mg/dL) | 0.87 | 0.47 | 3 | 0.33 | 0.15 | 3 | 0.45 | 0.07 | 2 | 0.20 | 0.00 | 2 | ND | ND | ND |
| BUN (mg/dL) | 23.0 | 2.6 | 3 | 21.0 | 3.5 | 3 | 24.7 | 4.6 | 3 | 21.3 | 2.9 | 3 | 18.7 | 7.2 | 3 |

TABLE 4
Hematology and serum chemistry parameters of C57BL/6 mice
on Study Day 21 after two doses of TGFRt15-TGFRs.

| | Study Day 21 | | | | | | | | | | | |
|---|---|---|---|---|---|---|---|---|---|---|---|---|
| | TGFRt15-TGFRs | | | | | | | | | | | |
| | 3 mg/kg | | | 10 mg/kg | | | 50 mg/kg | | | 200 mg/kg | | |
| Parameters | Mean | SD | N | Mean | SD | N | Mean | SD | N | Mean | SD | N |
| WBC count (×10³/μL) | 5.37 | 3.13 | 3 | 5.63 | 0.75 | 3 | 6.37 | 2.02 | 3 | 31.45 | 40.38 | 2 |
| RBC count (×10⁶/μL) | 6.37 | 1.67 | 3 | 7.45 | 0.62 | 3 | 6.82 | 0.67 | 3 | 7.13 | 0.18 | 2 |
| Hemoglobin (g/dL) | 9.0 | 2.1 | 3 | 10.1 | 0.8 | 3 | 9.7 | 0.7 | 3 | 10.5 | 0.8 | 2 |
| Hematocrit (%) | 30.3 | 7.2 | 3 | 35.6 | 3.5 | 3 | 33.7 | 2.2 | 3 | 36.2 | 3.5 | 2 |
| MCV(fL) | 47.7 | 2.3 | 3 | 47.7 | 1.2 | 3 | 49.7 | 2.1 | 3 | 50.5 | 3.5 | 2 |
| MCH (pg) | 14.0 | 1.0 | 3 | 13.3 | 0.6 | 3 | 14.3 | 0.6 | 3 | 14.5 | 0.7 | 2 |
| MCHC (%) | 30.0 | 0.0 | 3 | 28.3 | 1.5 | 3 | 28.7 | 0.6 | 3 | 28.5 | 0.7 | 2 |
| Neutrophils (×10³/μL) | 0.65 | 0.50 | 3 | 0.62 | 0.07 | 3 | 1.10 | 0.55 | 3 | 6.78 | 9.09 | 2 |
| Lymphocytes (×10³/μL) | 4.58 | 2.62 | 3 | 4.81 | 0.61 | 3 | 4.88 | 1.20 | 3 | 20.75 | 25.82 | 2 |
| Monocytes (×10³/μL) | 0.13 | 0.08 | 3 | 0.19 | 0.06 | 3 | 0.24 | 0.13 | 3 | 3.32 | 4.65 | 2 |
| Eosinophils (×10³/μL) | 0.01 | 0.01 | 3 | 0.02 | 0.03 | 3 | 0.12 | 0.12 | 3 | 0.62 | 0.83 | 2 |
| Basophils (×10³/μL) | 0.00 | 0.00 | 3 | 0.00 | 0.00 | 3 | 0.03 | 0.05 | 3 | 0.00 | 0.00 | 2 |
| Platelet count (×10³/μL) | 531.3 | 413.1 | 3 | 806.3 | 125.2 | 3 | 778.0 | 34.9 | 3 | 711.5 | 44.5 | 2 |
| % Neutrophils | 10.3 | 6.0 | 3 | 11.0 | 1.0 | 3 | 16.7 | 2.9 | 3 | 17.0 | 7.1 | 2 |
| % Lymphocytes | 87.0 | 6.0 | 3 | 85.3 | 1.5 | 3 | 77.7 | 5.1 | 3 | 75.5 | 14.8 | 2 |
| % Monocytes | 2.33 | 0.58 | 3 | 3.33 | 0.58 | 3 | 3.67 | 1.53 | 3 | 6.00 | 7.07 | 2 |
| % Eosinophils | 0.33 | 0.58 | 3 | 0.33 | 0.58 | 3 | 1.67 | 1.15 | 3 | 1.50 | 0.71 | 2 |
| % Basophils | 0.00 | 0.00 | 3 | 0.00 | 0.00 | 3 | 0.33 | 0.58 | 3 | 0.00 | 0.00 | 2 |
| AST (U/L) | 108.3 | 76.8 | 3 | 62.3 | 5.0 | 3 | 560.7^{a} | 888.2 | 3 | 198.5 | 190.2 | 2 |
| ALT (U/L) | 49.3 | 17.7 | 3 | 51.0 | 12.5 | 3 | 57.7 | 3.5 | 3 | 48.0 | 9.9 | 2 |
| Alkaline Phos. (U/L) | 110.3 | 12.4 | 3 | 121.0 | 18.0 | 3 | 174.7 | 99.4 | 3 | 138.0 | 5.7 | 2 |
| Total Bilirubin (mg/dL) | 0.57 | 0.12 | 3 | 0.47 | 0.15 | 3 | 0.45 | 0.07 | 2 | 0.65 | 0.07 | 2 |
| BUN (mg/dL) | 27.0 | 5.0 | 3 | 22.3 | 4.2 | 3 | 24.3 | 2.1 | 3 | 25.0 | 1.4 | 2 |

^{a}One of three mice in 50 mg/kg TGFRt15-TGFRs group had an observed AST value of 1586 U/L (~6 × ULN). This mouse did not show clinical signs and its ALT value (61 U/L) was within the normal range.

### Example 48: Sequestration of TGF-β by TGFRt15-TGFRs and TGFRt15*-TGFRs in Mice

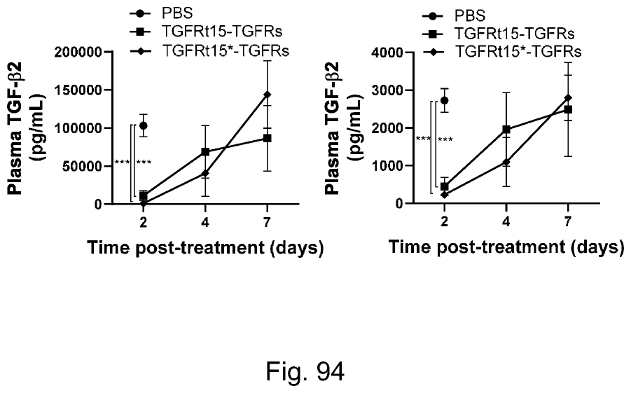

Female C57BL/6 mice were injected subcutaneously with PBS or 3 mg/kg of TGFRt15-TGFRs or TGFRt15*-TGFRs and plasma was collected at various times post-treatment. Plasma levels of TGF-β1 and TGF-β2 were determined using the TGFβ 3-Plex assay (Eve Technologies, Calgary, AL, Canada). TGFRt15-TGFRs and TGFRt15*-TGFRs were found to significantly decrease plasma TGF-β1 and TGF-β2 levels in C57BL/6 mice 2 days after treatment (FIG. 94), consistent with the activity of the TGFβRII domains of these fusion proteins.

### Example 49: Effects of TGFRt15-TGFRs and TGFRt15*-TGFRs on Immune Cell Metabolism In Vivo and In Vitro

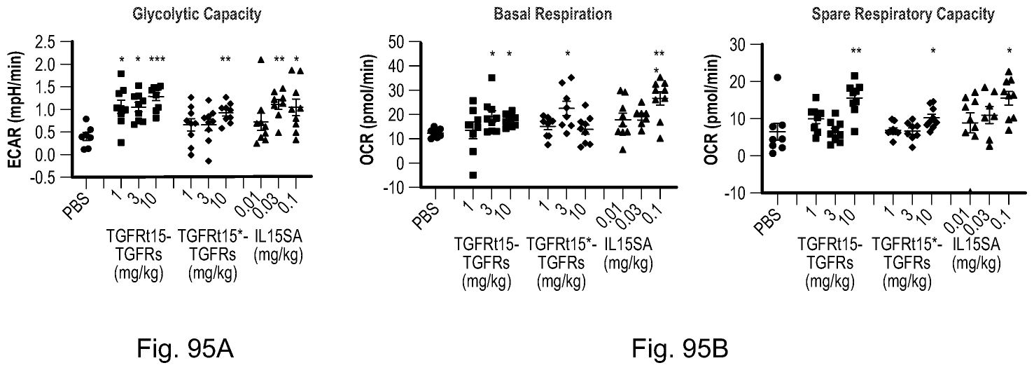

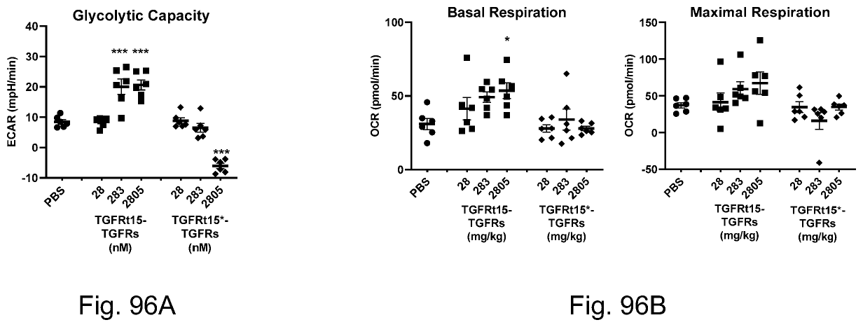

To assess treatment mediated effects on immune cell metabolism, extracellular flux assays were performed on splenocytes isolated from mice 4 days after PBS, TGFRt15-TGFRs, TGFRt15*-TGFRs or IL-15/IL-15R (IL15SA) administration. Extracellular flux assays on mouse splenocytes were performed using a XFp Analyzer (Seahorse Bioscience). As expected, TGFRt15-TGFRs and IL-15 increased the rates of glycolytic capacity (ECAR) (FIG. 95A) and mitochondrial respiratory capacity (OCR) (FIG. 95B) of the isolated splenocytes in a dose-level-dependent manner. In vivo TGFRt15*-TGFRs treatment also increased ECAR and OCR of splenocytes. This phenomenon was not observed when splenocytes from untreated C57BL/6 mice were incubated 4 days with TGFRt15*-TGFRs in vitro. Only TGFRt15-TGFRs (but not TGFRt15*-TGFRs) was capable of increasing splenocyte ECAR and OCR in vitro at physiologically relevant concentrations (FIGS. 96A-96B). This suggests that both the IL-15 and TGFβRII domains of TGFRt15-TGFRs have a role in stimulating immune cell metabolism in vivo.

### Example 50: Antitumor Efficacy of TGFRt15-TGFRs and TGFRt15*-TGFRs Against B16F10 Melanoma in C57BL/6 Mice

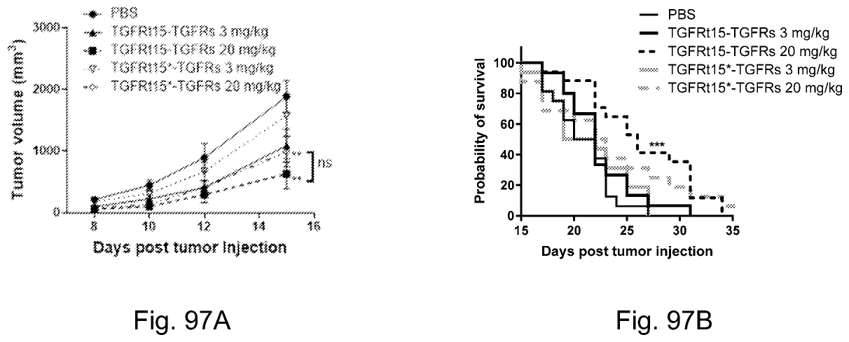

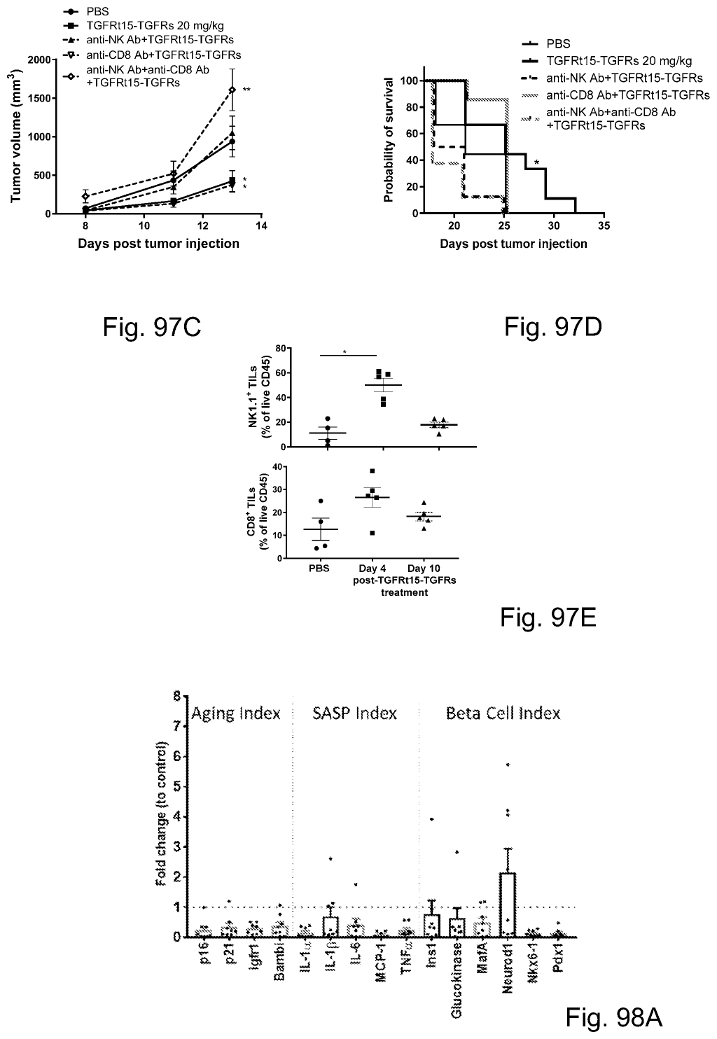

To evaluate TGFRt15-TGFRs and TGFRt15*-TGFRs antitumor efficacy, the murine B16F10 tumor model was selected as it is highly aggressive, poorly immunogenic and devoid of immune infiltrates, expresses TGF-β which plays a role in its growth and is resistant to cytokine and checkpoint blockade immunotherapies. B16F10 melanoma cells (5×10⁵ cells) (CRL-6475, ATCC) were subcutaneously injected into C57BL/6 mice followed by subcutaneous injection of PBS, TGFRt15-TGFRs (3 or 20 mg/kg) or TGFRt15*-TGFRs (3 or 20 mg/kg) on day 1 and 4 after tumor implantation. Tumor volume was measured every other day and mice with tumors≥4000 mm³ were sacrificed per IACUC regulation. Mouse survival was also assessed throughout the study period. When compared through SD15 (i.e., prior to animal mortality), treatment with TGFRt15-TGFRs or TGFRt15*-TGFRs at 20 mg/kg resulted in significantly slower tumor growth than was observed in the PBS treated mice (FIG. 97A). Tumor-bearing mice treated with 20 mg/kg TGFRt15-TGFRs also showed prolonged survival when compared to the 3 mg/kg TGFRt15-TGFRs and PBS treatment groups (FIG. 97B). These results indicate that TGFRt15-TGFRs and TGFRt15*-TGFRs have antitumor activity against solid B16F10 melanoma tumors with the bifunctional TGFRt15-TGFRs complex exhibiting the greater efficacy. Thus, both the TGFβRII and IL-15/IL-15RαSu domains play a role in TGFRt15-TGFRs-mediated activity against B16F10 tumors.

TGFRt15-TGFRs treatment is capable of significantly increasing the number of NK and T cells in vivo. To determine if these immune cells were responsible for TGFRt15-TGFRs-mediated antitumor efficacy, Ab immunodepletion of CD8⁺ T cells and NK1.1⁺ cells was conducted in tumor-bearing mice prior to TGFRt15-TGFRs treatment. It was found that NK1.1⁺ cell depletion (alone or in combination with CD8⁺ T cell depletion) eliminated the antitumor effects of TGFRt15-TGFRs in B16F10 tumor-bearing mice during the first 2 weeks post-treatment (FIG. 97C), whereas either NK1.1⁺ cell depletion or CD8⁺ T cell depletion reduced the survival benefit seen with TGFRt15-TGFRs (FIG. 97D). Consistent with these findings, TGFRt15-TGFRs treatment also promoted an increase in NK cell and CD8⁺ T cell infiltration into B16F10 tumors (FIG. 97E). These results support the conclusion that both CD8⁺ T cells and NK cells play a major role in TGFRt15-TGFRs-mediated activity against melanoma tumor cells in C57BL/6 mice.

### Example 51: TGFRt15-TGFRs Significantly Down-Regulated Aging Index and SASP Index

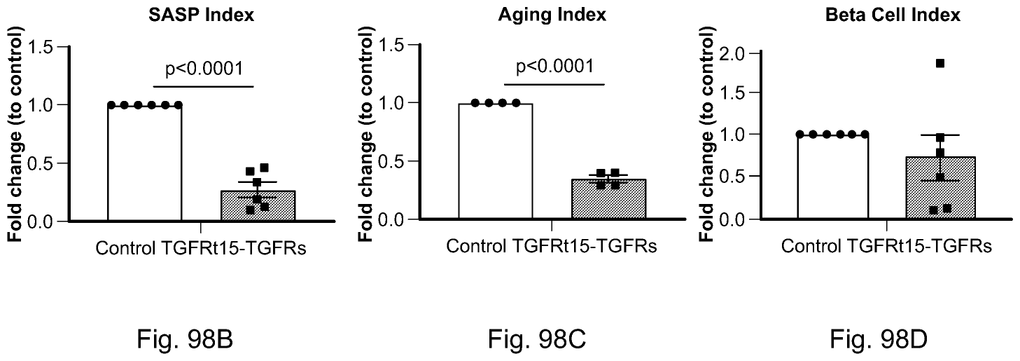

Five-week-old male BKS.Cg-Dock7m+/+Leprdb/J (db/db) mice were fed with standard chow diet and received drinking water ad libitum. At the age of six weeks, mice were randomly assigned to control and treatment groups (n=5/group). The treatment group received TGFRt15-TGFRs by subcutaneous injection at 3 mg/kg at weeks 6 and 12 from the start of the study, while the control group received vehicle (PBS) only. At end of study (4-weeks post the 2^{nd} dose), mice were euthanized and pancreas was collected. The half of pancreas was homogenized with the TRIzol reagent (Invitrogen) and total tissue RNA was purified with RNeasy Mini Kit (Qiagen). Synthesis of cDNA was performed using a QuantiTect Reverse Transcription Kit (Qiagen) and quantitative PCR was performed using a SsoAdvanced™ Universal SYBR® Green Supermix (BioRad) and a QuantiStudio 3 Real-Time PCR System (Applied Biosystems) according to comparative threshold cycle method following manufacturer's protocol. The amplification reactions were performed in duplicate, and the fluorescence curves were analyzed with the software included with the QuantiStudio 3 Real-Time PCR System. The housekeeping gene 18s ribosomal RNA was used as an endogenous control reference. The expression of each target mRNA relative to 18s rRNA was calculated based on Ct as 2^{-Δ(Δct)}, in which ΔCt=Ct_{target}−Ct_{18S}. As shown in FIG. 98A, TGFRt15-TGFRs treatment of db/db mice resulted in a reduction of pancreatic gene expression for p16, p21, Igfr1, and Bamb1 of the Aging gene index and IL-1α, IL-6, MCP-1, and TNFα of SASP gene index when compared to the control group. Generally, pancreatic expression of genes of the SASP Index and Aging Index were significantly reduced following TGFRt15-TGFRs treatment compared to controls, whereas pancreatic gene expression of the beta cell index was not changed significantly in the TGFRt15-TGFRs and PBS-treated db/db mice. (FIGS. 98B, 98C, 98D). The data suggested TGFRt15-TGFRs has potent senolytic and senomorphic activities to reduce senescent cells and SASP factors in the pancreas of db/db mice.

### Example 52: TGFRt15-TGFRs Reduced Senescent Cells of Pancreatic Beta Cells

Five-week-old male BKS.Cg-Dock7m+/+Leprdb/J (db/db) mice (Jackson Lab) were fed with standard chow diet (Irradiated 2018 Teklad global 18% protein rodent diet, Envigo) and received drinking water ad libitum. At the age of six weeks, mice were randomly assigned to control and treatment groups (n=5/group). The treatment group received TGFRt15-TGFRs by subcutaneous injection at 3 mg/kg at weeks 6 and 12 from the start of the study, while control group received vehicle (PBS) only. At end of study (4-weeks post the 2^{nd} dose), mice were euthanized and pancreata were removed en bloc, immersion-fixed in 4% formaldehyde (4% formaldehyde in 0.1M phosphate buffer; PBS pH 7.4) and stored at 4° C. degrees until further processing. Dissected pancreata were paraffinized, embedded, and sectioned, and three 10 mm sections (150 mm apart) were cut from each block representing in total a systematic uniform random sample of the whole pancreas from each animal.

Multispectral imaging was performed using the Akoya Vectra Polaris instrument. This instrumentation allows for phenotyping, quantification, and spatial relationship analysis of tissue infiltrate in formalin-fixed paraffin-imbedded biopsy sections. To quantify levels of p21 in insulin⁺ islet regions of the pancreas, formalin-fixed paraffin-embedded tissue sections were stained consecutively with specific primary antibodies according to standard protocols provided by Akoya and performed routinely by the HIMSR. Briefly, the slides were deparaffinized, heat treated in antigen retrieval buffer, blocked, and incubated with rabbit primary antibodies against insulin (#4590, Cell Signaling Technology) and p21 (EPR362, Abcam), followed by horseradish peroxidase (HRP)-conjugated secondary antibody polymer (anti-rabbit), and HRP-reactive OPAL fluorescent reagents (OPAL-520 for insulin and OPAL-570 for p21, Akoya) that use TSA chemistry to deposit dyes on the tissue immediately surrounding each HRP molecule. To prevent further deposition of fluorescent dyes in subsequent staining steps, the slides were stripped in between each stain with heat treatment in antigen retrieval buffer (Citrate buffer for insulin and EDTA buffer for p21). Whole slide scans were collected with the Akoya Vectra Polaris instrument using the 20× objective with a 0.5 micron resolution. The 3 color images were analyzed with inForm software (Akoya) to unmix adjacent fluorochromes, subtract autofluorescence, segment insulin⁺ regions of the tissue, compare the frequency and location of cells, segment cellular cytoplasmic and nuclear regions, and phenotype infiltrating cells according to cell marker expression.

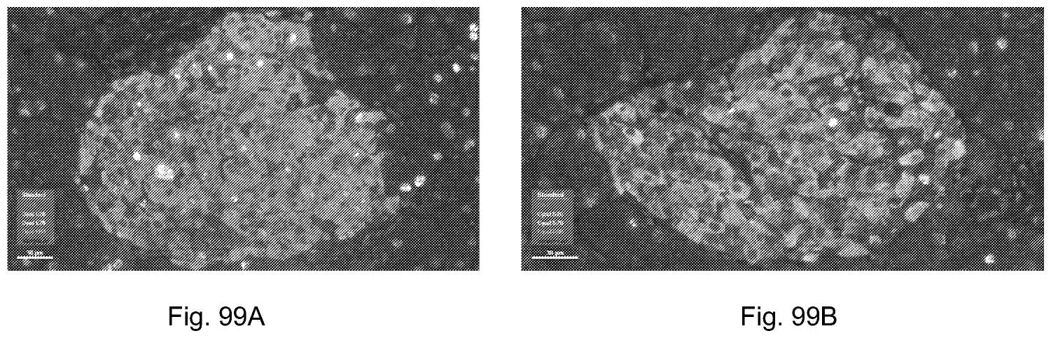

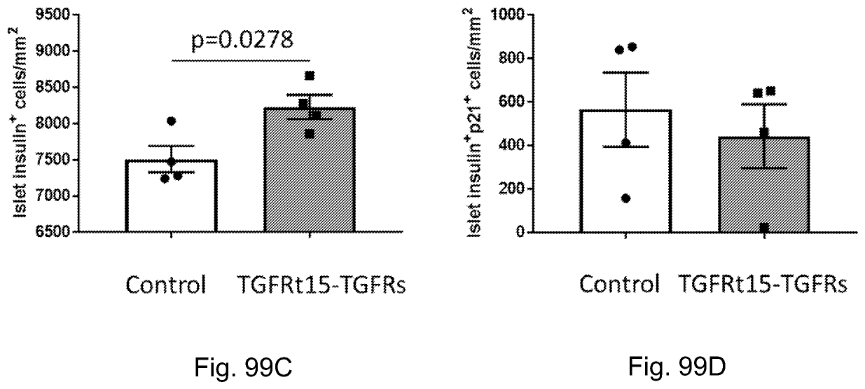

As shown in FIG. 99A-99D, p21 positive senescent cells (OPAL-570) were accumulated more in insulin positive islet beta cells (OPAL-520) in pancreas of control group (FIG. 99A) and these senescent cells were reduced in pancreas of TGFRt15-TGFRs treatment group (FIG. 99B). The insulin positive islet cells were significantly increased in TGFRt15-TGFRs treatment group compared with the control group (p=0.0278, FIG. 99C). The p21 positive senescent beta cells (insulin positive) were reduced in TGFRt15-TGFRs treated group compared with the control group though the difference was not statistically significant (FIG. 99D). Overall, the data suggested TGFR15-TGFRs has senolytic activity to remove senescent cells and promotes the recovery of normal functional islet beta cells in the pancreas of db/db mice.

### Example 53: TGFRt15-TGFRs Reduced Senescent Cells of Pancreatic Beta Cells by Increasing NK, NKT, and CD8⁺ T Cells

Five-week-old male BKS.Cg-Dock7m+/+Leprdb/J (db/db) mice (Jackson Lab) were fed with standard chow diet (Irradiated 2018 Teklad global 18% protein rodent diet, Envigo) and received drinking water ad libitum. At the age of six weeks, mice were randomly assigned into control and treatment groups (n=5/group). The treatment group received TGFRt15-TGFRs by subcutaneous injection at 3 mg/kg at weeks 6 and 12 from the start of the study, while control group received vehicle (PBS) only.

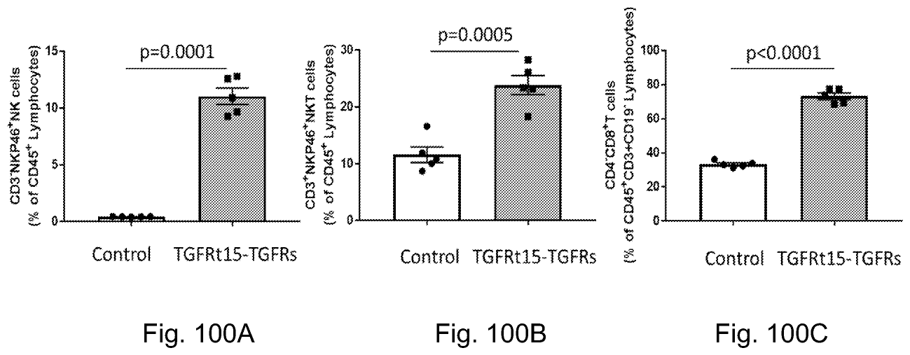

Four days after the 1^{st} dose treatment, blood was collected and whole blood cells (50 mL) were treated with ACK (Ammonium-Chloride-Potassium) lysing buffer to lyse red blood cells. The lymphocytes were then stained with PE-Cy7-anti-CD3, BV605-anti-CD45, PerCP-Cy5.5-anti-CD8a, BV510-anti-CD4, and APC-anti-NKD46 antibodies (all antibodies from BioLegend) to assess the population of T cells, NKT cells, and NK cells. As shown in FIG. 100A-100C, the percentages of CD8⁺ T cells, CD3⁺NKP46⁺ NKT cells, and CD3⁺NKP46⁺ NK cells increased in the blood of db/db mice following treatment with TGFRt15-TGFRs compared to the PBS-treated mice.

### Example 54: Phenotyping of Immune Cell Subsets in Peripheral Blood of Cynomolgus Monkeys Following Administration of TGFRt15-TGFRs

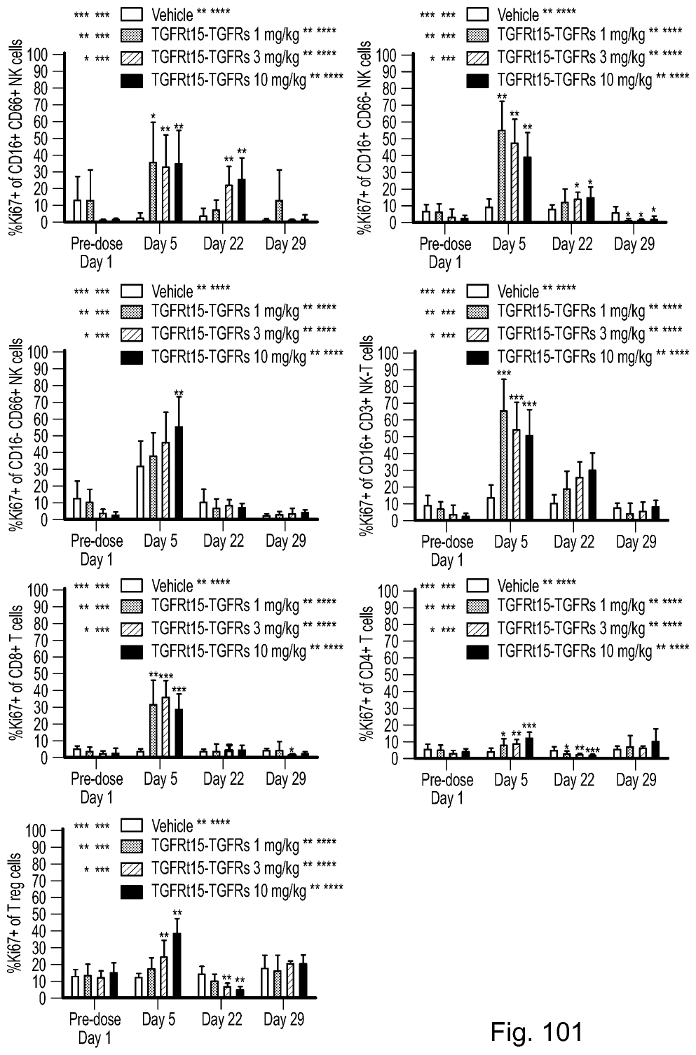

Cynomolgus monkeys (5M: 5F per group) were treated subcutaneously with PBS (vehicle) or TGFRt15-TGFRs at 1, 3 or 10 mg/kg on study days 1 and 15. Blood was collected pre-day (day 1) and days 5, 22 and 29 post-treatment. PBMCs were prepared and stained with a panel of fluor-conjugated antibodies to assess the phenotypes of B cells, NK cells, NK-T cells, Treg cells and CD4⁺ and CD8⁺ T cells by flow cytometry. FIG. 101 shows that TGFRt15-TGFRs administration resulted in a significant increase in the percentage of Ki67 NK cells, NK-T cells, Treg cells and CD4⁺ and CD8⁺ T cells on day 5 post-treatment. These findings indicate that TGFRt15-TGFRs treatment induced proliferation of these lymphocyte subsets in non-human primates. No treatment effects were observed on Ki67 expression in B cells.

### Example 55: IL-15 Immunostimulatory and TGF-β Antagonist Activities of TGFRt15-TGFRs

Six-week-old (young) and 72-week-old (aged) C57BL/6 mice were subcutaneously injected with single dose of PBS, TGFRt15-TGFRs (3 mg/kg) or TGFRt15*-TGFRs (3 mg/kg). On day 4 after treatment, mice were sacrificed, and the spleens were harvested. The spleens were crushed with flat back end of the sterile piston/plunger of 3 cc syringe to release the splenocytes. The splenocytes were passed through a 70 μM cell strainer and homogenized into a single cell suspension. The RBCs were lysed in ACK lysis buffer and the splenocytes were washed and counted. To measure the glycolytic activity of the splenocytes, the cells were washed and resuspended in Seahorse media and resuspended at 4×10⁶ cells/mL. Cells were seeded at 50 μL/well in Cell-Tak-coated Seahorse Bioanalyzer XFe96 culture plates in Seahorse XF RPMI medium, pH 7.4 supplemented with 2 mM L-glutamine for glycolysis stress test. The cells were allowed to attach to the plate for 30 min at 37° C. Additionally, 130 μL of the assay medium was added to each well of the plate (also the background wells). The plate was incubated in 37° C., non-CO₂ incubator for 1 hr. For glycolysis stress test the calibration plate contained 10× solution of glucose/oligomycin/2DG prepared in Seahorse assay media and 20 μL of glucose/oligomycin/2DG were added to each of the ports of the extracellular flux plate that was calibrated overnight. The glycolysis stress test is based on extracellular acidification rate (ECAR) and measures three key parameters of glycolytic function including glycolysis, glycolytic capacity and glycolytic reserve. Complete ECAR analysis consisted of four stages: non glycolytic acidification (without drugs), glycolysis (10 mM glucose), maximal glycolysis induction/glycolytic capacity (2 μM oligomycin), and glycolysis reserve (100 mM 2-DG). At the end of the experiment the data was exported as a Graph Pad Prism file. The XF glycolysis stress test report generator automatically calculated the XF cell glycolysis stress test parameters from the Wave data. The data was analyzed using the Wave software (Agilent).

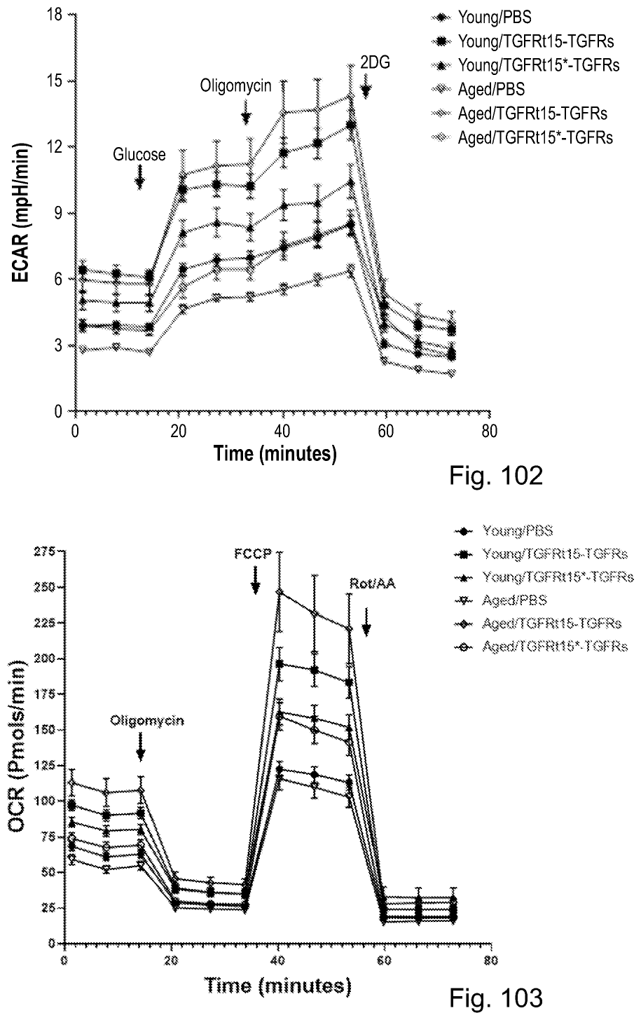

As shown in FIG. 102, the splenocytes isolated from aged mice on day 4 after TGFRt15-TGFRs treatment showed enhanced basal glycolysis, glycolysis capacity, and glycolysis reserve rates, when compared to splenocytes of the PBS or TGFRt15*-TGFRs treatment groups. The glycolytic function of splenocytes of aged control mice was less than that of the young control mice. Treatment of young and aged mice with TGFRt15*-TGFRs was capable of increasing splenocyte glycolytic function. However, TGFRt15-TGFRs treatment of aged mice was able to increase the rates of splenocyte basal glycolysis, glycolysis capacity, and glycolysis reserve to levels equivalent to those observed in the splenocytes from TGFRt15-TGFRs treated young mice. These findings suggest that the IL-15 immunostimulatory and TGF-β antagonist activities of TGFRt15-TGFRs effectively stimulate and rejuvenate the diminished metabolic activity of immune cells from aged mice.

Six-week-old (young) and 72-week-old (aged) C57BL/6 mice were subcutaneously injected with single dose of PBS, TGFRt15-TGFRs (3 mg/kg) or TGFRt15*-TGFRs (3 mg/kg). On day 4 after treatment, mice were sacrificed, and the spleens were harvested. The spleens were crushed with flat back end of the sterile piston/plunger of 3 cc syringe to release the splenocytes. The splenocytes were passed through a 70 μM cell strainer and homogenized into a single cell suspension. The RBCs were lysed in ACK lysis buffer and the splenocytes were washed and counted. To measure the mitochondrial respiration of the splenocytes, the cells were washed and resuspended in Seahorse media and resuspended at 4×10⁶ cells/mL. Cells were seeded at 50 μL/well in Cell-Tak-coated Seahorse Bioanalyzer XFe96 culture plates in Seahorse XF RPMI medium, pH 7.4 supplemented with 2 mM L-glutamine for glycolysis stress test. For mitochondrial stress test, the cells were seeded in Seahorse XF RPMI medium, pH 7.4 supplemented with 10 mM glucose and 2 mM L-glutamine. The cells were allowed to attach to the plate for 30 min at 37° C. Additionally, 130 μL of the assay medium was added to each well of the plate (also the background wells). The plate was incubated in 37° C., non-CO₂ incubator for 1 hr. For mitochondrial stress test, the calibration plate contained 10× solution of oligomycin/FCCP/rotenone prepared in Seahorse assay media and 20 μL of oligomycin, FCCP and rotenone was added to each of the ports of the extracellular flux plate that was calibrated overnight. Oxygen consumption rate (OCR) was measured using an XFe96 Extracellular Flux Analyzer. Complete OCR analysis consisted of four stages: basal respiration (without drugs), ATP-linked respiration/Proton leak (1.5 M oligomycin), maximal respiration (2 μM FCCP), and spare respiration (0.5 μM rotenone). At the end of the experiment, the data was exported as a Graph Pad Prism file. The XF mitochondrial stress test report generator automatically calculates the XF mitochondrial stress test parameters from the Wave data that have been exported to Excel. The data was analyzed by using the Wave software (Agilent).

As shown in FIG. 103, the splenocytes isolated from aged mice on day 4 after TGFRt15-TGFRs therapy showed enhanced basal respiration, ATP-linked respiration, maximal respiration, and reserve capacity, when compared to splenocytes of the PBS or TGFRt15*-TGFRs treatment groups. Treatment of young and aged mice with TGFRt15*-TGFRs was capable of increasing splenocyte mitochondrial respiration. However, TGFRt15-TGFRs treatment in aged mice able to increase the rates of basal respiration, ATP-linked respiration, maximal respiration, and reserve capacity to levels equivalent or higher to those observed in the splenocytes from TGFRt15-TGFRs treated young mice. These findings suggest that the IL-15 immunostimulatory and TGF-β antagonist activities of TGFRt15-TGFRs effectively stimulate and rejuvenate the diminished metabolic activity of immune cells from aged mice.

### Example 56: IL-15 Activity of TGFRt15-TGFRs Plays a Role in Increasing CD8⁺ T Cells and NK Cells

Six-week-old (young) and 72-week-old (aged) C57BL/6 mice were purchased from the Jackson Laboratory. Mice were housed in a temperature and light controlled environment. Mice (n=6/group) were treated subcutaneously with PBS, TGFRt15-TGFRs (3 mg/kg) and TGFRt15*-TGFRs (3 mg/kg). The mouse blood was collected from submandibular vein on day 4 post treatment in tubes containing EDTA to evaluate changes in the different subsets of immune cells. Whole blood RBCs were lysed in ACK buffer for 5 minutes at room temperature. Remaining cells were washed in FACS buffer (1×PBS (Hyclone) with 0.5% BSA (EMD Millipore) and 0.001% sodium azide (Sigma)). To assess the different types of immune cells in blood, cells were stained with antibodies specific to cell-surface CD3, CD4, CD45, CD8 and NK1.1 (BioLegend) for 30 min at room temperature (RT). After surface staining, cells were washed (1500 RPM for 5 min at RT) in FACS buffer (1×PBS (Hyclone) with 0.5% BSA (EMD Millipore) and 0.001% sodium azide (Sigma)). After two washes, cells were resuspended in fixation buffer and analyzed by flow cytometry (Celesta-BD Bioscience).

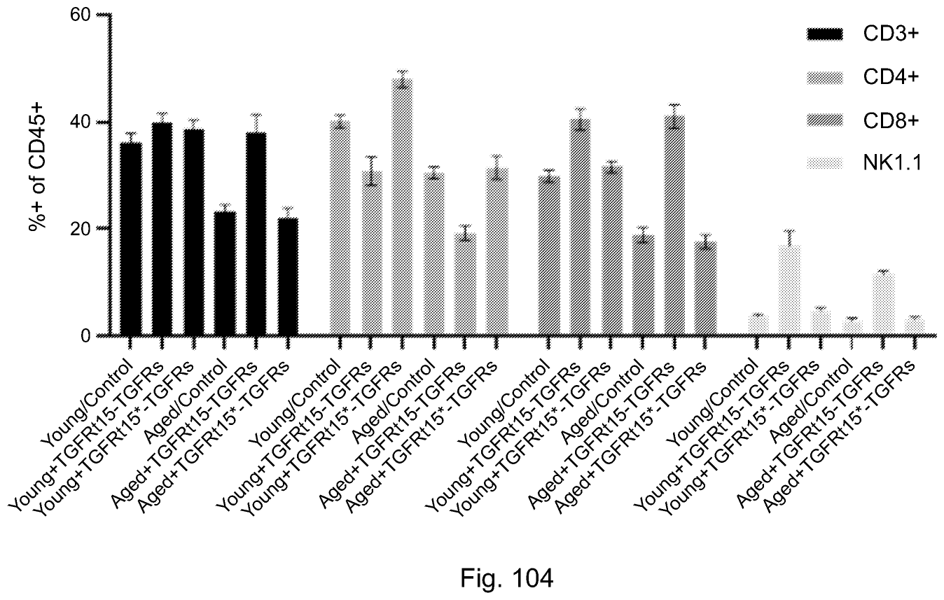

As shown in FIG. 104, the results indicate that treatment of aged mice with TGFRt15-TGFRs induced an increase in the percentages of CD3 CD45⁺, CD3⁺CD8⁺, and CD3⁻NK1.1⁺ immune cells in the blood, whereas treatment of aged mice with TGFRt15*-TGFRs had no effect on the percentage of these blood cell populations. These results suggest that IL-15 activity of TGFRt15-TGFRs plays a role in increasing CD8⁺ T cells and NK cells in the blood of aged mice. The percentage of blood T cells and NK cells in aged control mice was less than that of the young control mice. However, treatment of aged mice with TGFRt15-TGFRs increased the percentages of CD3⁺CD45⁺, CD3⁺CD8⁺, and CD3 NK1.1+ immune cells in the blood to levels similar to those observed in the blood of TGFRt15-TGFRs treated young mice.

Six-week-old (young) and 72-week-old (aged) C57BL/6 mice were purchased from the Jackson Laboratory. Mice were housed in a temperature and light controlled environment. Mice (n=6/group) were treated subcutaneously with PBS, TGFRt15-TGFRs (3 mg/kg) and TGFRt15*-TGFRs (3 mg/kg). Four days after treatment, the mice were euthanized, and spleen was harvested and processed to a single cell suspension. Single cells suspension was prepared in order to evaluate the different subsets of immune cells. RBCs were lysed in ACK buffer for 5 min at room temperature. The remaining cells were washed in FACS buffer (1×PBS (Hyclone) with 0.5% BSA (EMD Millipore) and 0.001% sodium azide (Sigma)). To assess the different types of immune cells in spleen, cells were stained with antibodies specific to cell-surface CD3, CD45, CD8 and NK1.1 (BioLegend) for 30 minutes at RT. After surface staining, cells were washed (1500 RPM for 5 min at room temperature) in FACS buffer (1×PBS (Hyclone) with 0.5% BSA (EMD Millipore) and 0.001% sodium azide (Sigma)). After two washes, cells were resuspended in fixation buffer and analyzed by flow cytometry (Celesta-BD Bioscience).

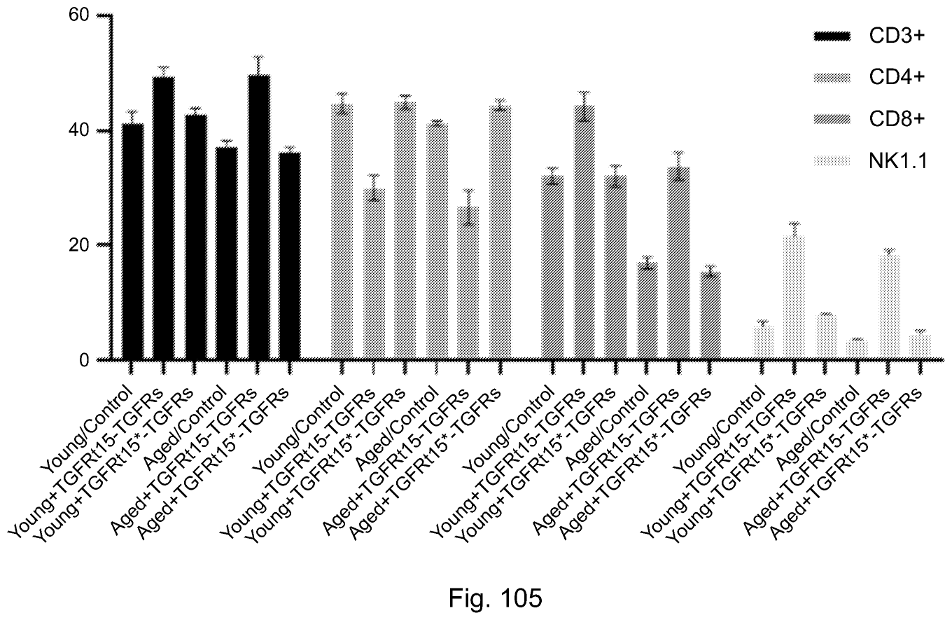

As shown in FIG. 105, the results indicate that treatment of aged mice with TGFRt15-TGFRs induced an increase in the percentages of CD3 CD45⁺, CD3⁺CD8⁺, and CD3 NK1.1+ immune cells in the spleen, whereas treatment of aged mice with TGFRt15*-TGFRs had no effect on the percentage of these splenocyte populations. These results suggest that IL-15 activity of TGFRt15-TGFRs plays a role in increasing CD8⁺ T cells and NK cells in the blood of aged mice. The percentage of spleen T cells and NK cells in aged control mice was less than that of the young control mice. However, treatment of aged mice with TGFRt15-TGFRs increased the percentages of CD3⁺CD45⁺, CD3⁺CD8⁺, and CD3 NK1.1+ immune cells in the spleen to levels similar to those observed in the spleen of TGFRt15-TGFRs treated young mice.

### Example 57: TGFRt15-TGFRs-Associated Decrease in Naturally-Occurring Senescent Cells in the Liver

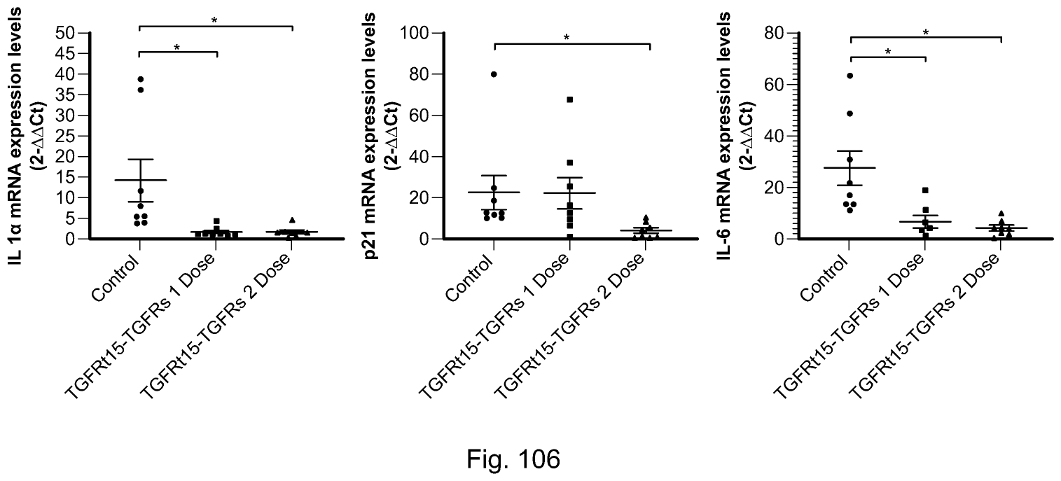

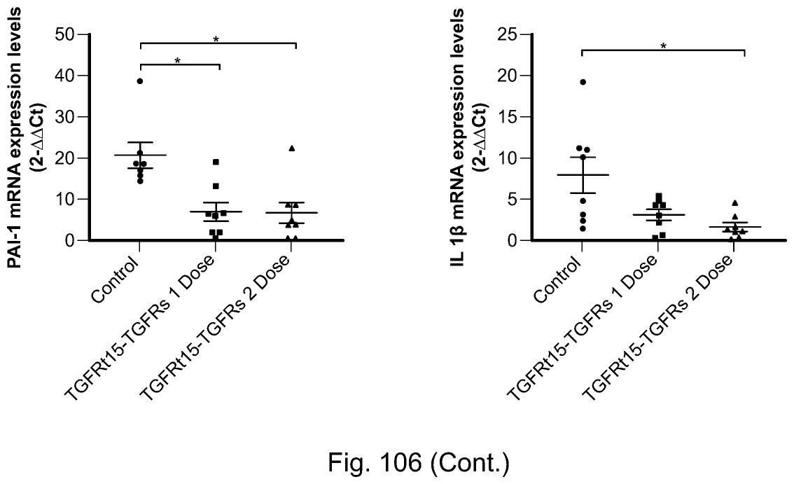

Seventy-two-week-old (aged) C57BL/6 mice were purchased from the Jackson Laboratory. Mice were housed in a temperature and light controlled environment. Mice (n=8/group) were treated subcutaneously with either PBS or one dose or two doses (at day 0 and 60) of TGFRt15-TGFRs (3 mg/kg). On day 71 post treatment, mice were euthanized and the livers were harvested and stored in liquid nitrogen in 1.7 mL Eppendorf tubes. Tissue samples were homogenized by using homogenizer in 1 mL of Trizol (Thermo Fischer). Homogenized tissues were transferred in fresh Eppendorf tubes and total RNA was extracted using RNeasy Mini Kit (Qiagen #74106) according to the manufacturer's instructions. One μg of total RNA was used for cDNA synthesis using the QuantiTect Reverse Transcription Kit (Qiagen). Real-time PCR was carried out with CFX96 Detection System (Bio-Rad) using FAM labeled predesigned primers purchased from Thermo Scientific. Reactions were run in triplicate for all the genes examined. The housekeeping gene 18S ribosomal RNA was used as an internal control to normalize the variability in gene expression levels. The expression of each target mRNA relative to 18S rRNA was calculated based on Ct as 2^{-Δ(Δct)}, in which ΔCt=Ct_{target}−Ct_{18S}. Untreated 6-week-old mice were used as a control to compare the gene expression level to aged mice. The results showed that gene expression of IL-1α, IL-1β, IL-6, p21 and PAI-1 in liver increased with the age of the mice as expected with the age-dependent increase in cellular senescence-associated transcripts. Treatment of 72-week-old mice with a single dose or two doses of TGFRt15-TGFRs resulted in a significant reduction in gene expression of senescence markers IL-1α, IL-1β, IL-6, p21 and PAI-1 in liver when compared to the PBS control group (FIG. 106). These findings suggest a TGFRt15-TGFRs-associated decrease in naturally-occurring senescent cells in the liver of aged mice.

### Example 58: TGFRt15-TGFRs Treatment is Capable of Reducing Inflammation in Liver Tissues

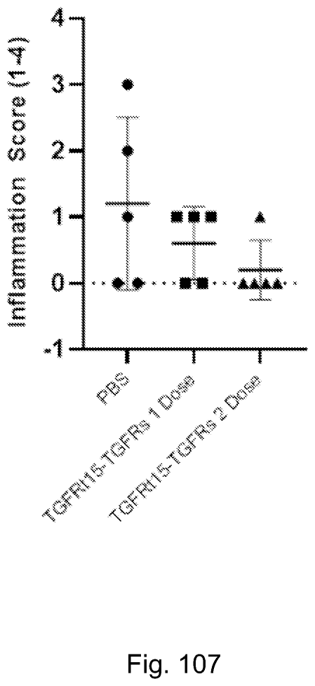

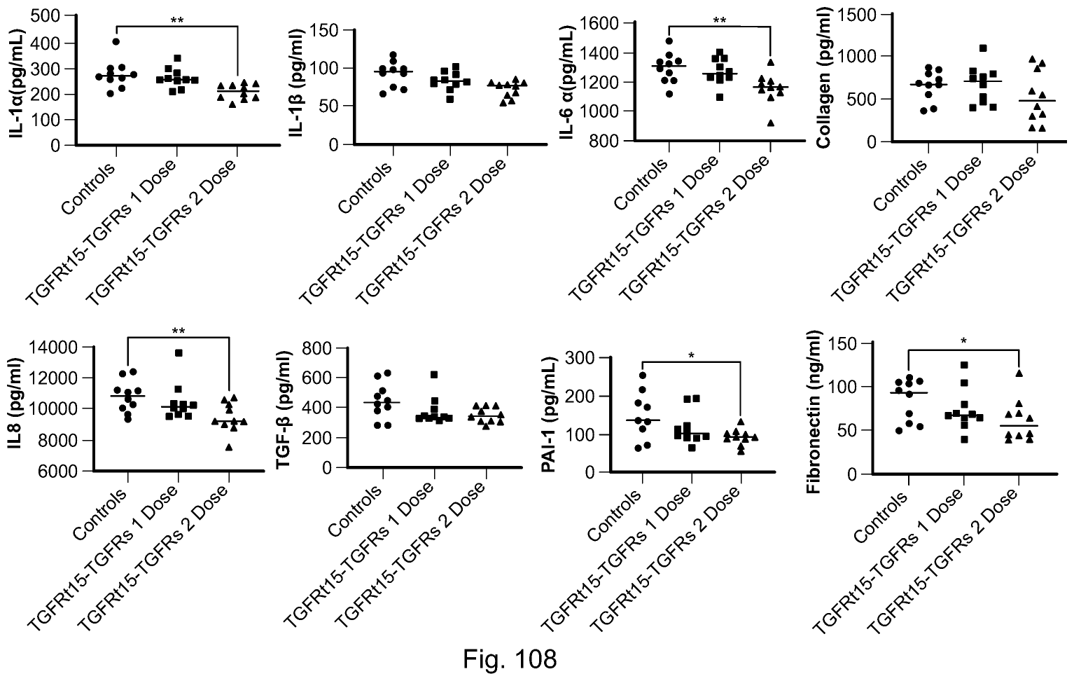

Seventy-two-week-old (aged) C57BL/6 mice were purchased from the Jackson Laboratory. Mice were housed in a temperature and light controlled environment. Mice (n=10/group) were treated subcutaneously with either PBS or one or two doses of TGFRt15-TGFRs (3 mg/kg). On day 120 after treatment, mice were euthanized and the mouse liver was prepared to evaluate by histochemistry. Liver tissue specimens were fixed in 10% formaldehyde and after a paraffin blocking procedure, cross-sections were stained with hematoxylin-eosin. The extent of liver injury was evaluated histologically in a blinded manner. Histological sections of whole liver areas were scores for inflammation using a scale from 0 to 4 (0, absent and appearing to be normal; 1, light; 2, moderate; 3, strong; and 4, intense). As shown in FIG. 107, two doses of TGFRt15-TGFRs decrease the liver inflammation score in liver of aged mice compared to single dose TGFRt15-TGFRs or PBS control groups. These results suggest that TGFRt15-TGFRs treatment is capable of reducing inflammation in liver tissues of aged mice.

### Example 59: TGFRt15-TGFRs Treatment can Reduce IL1-α, IL-6, IL-8, PAI-1 and Fibronectin Protein Levels

Seventy-two-week-old (aged) C57BL/6 mice were purchased from the Jackson Laboratory. Mice were housed in a temperature and light controlled environment. Mice (n=10/group) were treated with either PBS or one dose or two doses (at day 0 and 60) of TGFRt15-TGFRs (3 mg/kg). On day 120 after treatment, mice were euthanized and liver were harvested and stored in liquid nitrogen in 1.7 mL Eppendorf tubes. Tissue samples were homogenized by using homogenizer in 0.3 mL of extraction buffer (Abcam). Homogenized tissues were transferred in fresh Eppendorf tubes. Protein levels in homogenized tissue were quantified using BCA Protein Assay Kit (Pierce). An ELISA to detect IL-1α, IL-1β, IL-6, IL-8, TGF-β, PAI-1, collagen and fibronectin (R&D System) was performed using 25 μg of tissue homogenize. As shown in FIG. 108, protein levels of IL-1α, IL-6, IL-8, PAI-1 and fibronectin were reduced in liver of mice treated with 2 doses of TGFRt15-TGFRs compared to PBS control or one dose TGFRt15-TGFRs treatment groups. These results indicate that 2 doses of TGFRt15-TGFRs treatment can reduce IL-1α, IL-6, IL-8, PAI-1 and fibronectin protein levels in liver of aged mice. Protein levels of IL-1β, TGF-β and collagen were also lower in liver of mice treated with 2 doses of TGFRt15-TGFRs compared to PBS controls; however, these changes did not reach statistical significance.

### Example 60: TGFRt15-TGFRs Reduces Senescence Cells

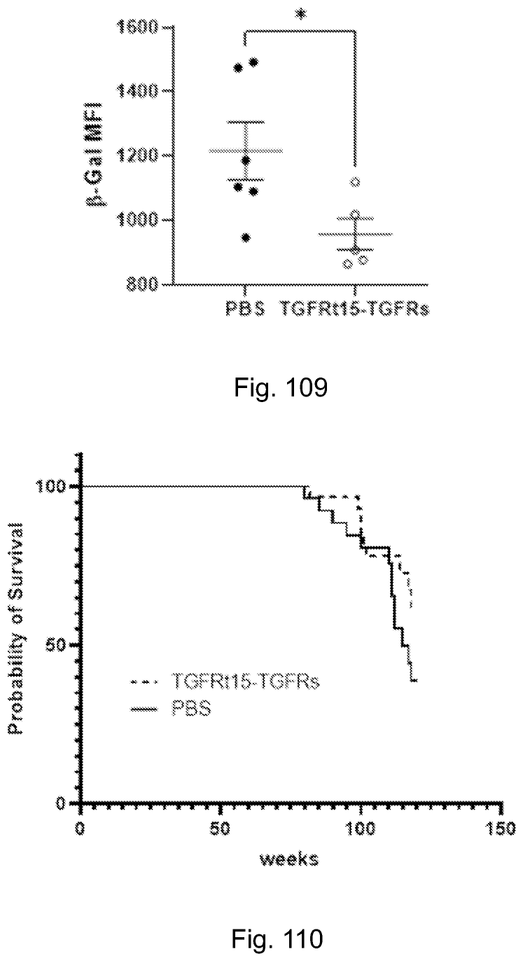

Seventy-two-week-old (aged) C57BL/6 aged mice which were purchased from the Jackson Laboratory. Mice were housed in a temperature and light controlled environment. Mice (n=5/group) were treated subcutaneously with either PBS or TGFRt15-TGFRs (3 mg/kg). On day 4 after treatment, mice were euthanized and livers were harvested, homogenized in PBS containing 2% FBS, and filtered in 70-micron filter to obtain a single cell suspension. Cells were spun down then resuspended in 5 mL RPMI containing 0.5 mg/mL collagenase IV and 0.02 mg/mL DNAse in 14 mL round bottom tubes. Cells were then shaken on orbital shaker for 1 hr at 37° C. and washed twice with RPMI. Cells were resuspended at 2×10⁶/mL in 24 wells flat bottom plate in 2 mL of complete media (RPMI 1640 (Gibco) supplemented with 2 mM L-glutamine (Thermo Life Technologies), penicillin (Thermo Life Technologies), streptomycin (Thermo Life Technologies), and 10% FBS (Hyclone)) and cultured for 48 hr at 37° C., 5% CO₂. Cells were harvested, washed once in warm complete media at 1000 rpm for 10 minutes at room temperature. Cell pellet was resuspended in 500 μL of fresh media containing 1.5 μL of Senescence Dye per tube (Abcam). Cells were further incubated for 1-2 hr at 37° C., 5% CO₂ and wash twice with 500 μL wash buffer. Cell pellet was resuspended in 500 μL of wash buffer and was analyzed immediately by flow cytometry (Celesta-BD Bioscience). As shown in FIG. 109, the percentage of senescence marker β-gal⁺ cells were decreased 4 days after in vivo treatment with TGFRt15-TGFR. These results demonstrate that TGFRt15-TGFRs is capable of reducing senescence cells (based on the β-gal marker) in liver of aged mice.

### Example 61: Effects of TGFRt15-TGFRs on Survival of Aged Mice

Seventy-two-week-old C57BL/6 mice were purchased from the Jackson Laboratory. Mice were housed in a temperature and light controlled environment. Mice were treated subcutaneously with either PBS or one dose of TGFRt15-TGFRs (3 mg/kg) (n=20/group). Mice were monitored every day for survival up to 120 weeks post treatment. The survival probability of the treatment groups based on the Mantel-Cox log-rank test is shown in FIG. 110. Compared with TGFRt15-TGFRs, higher mortality rates were found in control mice which was represented by a decline in the survival rates of the mice. By week 120 post treatment, there was a 70% mortality rate in PBS control mice compared to a 45% mortality rate in the TGFRt15-TGFRs-treated mice.

### Example 62: Effects of TGFRt15-TGFRs in Reducing SASP Factors in Liver of B16F10 Tumor-bearing Mice Following Chemotherapy

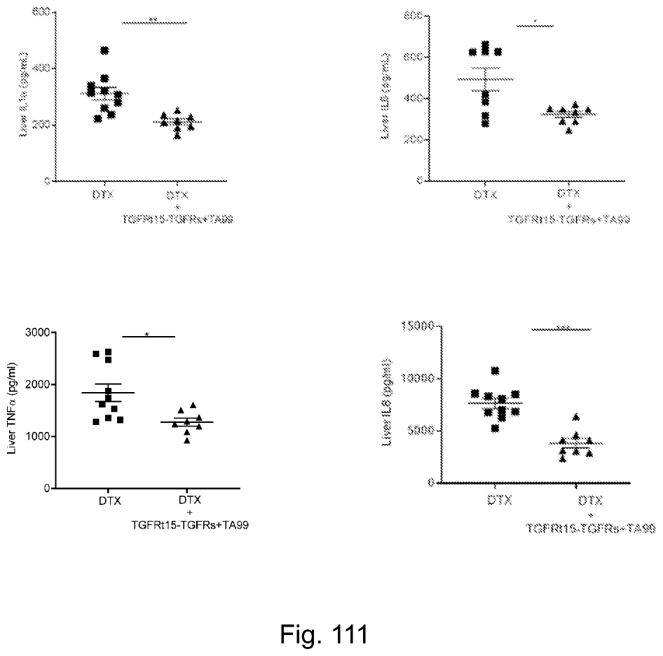

The effects of TGFRt15-TGFRs treatment in reducing protein levels of SASP factors in B16F10 tumor-bearing mice following chemotherapy were further assessed. B16F10 tumor cells (1×10⁷ cells/mouse) were implanted in mice on day 0. The mice were treated subcutaneously with 10 mg/kg docetaxel on days 1, 4, and 7. On day 8, the mice were treated subcutaneously with PBS or TGFRt15-TGFRs (3 mg/kg). Mice were euthanized on day 17 post-tumor inoculation and livers were collected and homogenized. Protein levels of SASP factors in the liver homogenates was determined by ELISA. As shown in FIG. 111, in vivo treatment with TGFRt15-TGFRs resulted in a significant reduction in levels of liver IL-1α, IL-6, TNFα and IL-8 SASP factors in B16F10 tumor bearing mice following chemotherapy.

### Example 63: Role of Immune Cell Subsets in TGFRt15-TGFRs-mediated

### Elimination of Senescent Tumor Cells in B16F10 Melanoma Mouse Model

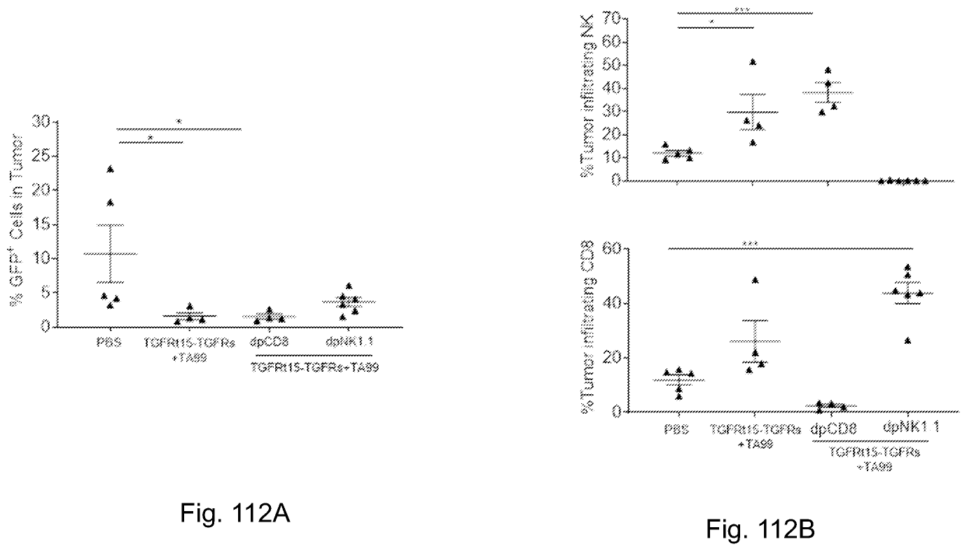

To assess the role of immune cell subsets in TGFRt15-TGFRs-mediated senescent-tumor-cell elimination, in vitro-docetaxel induced senescent B16F10-GFP tumor cells were mixed with parental B16F10 cells were implanted subcutaneously in mice following treatment with anti-NK1.1 or anti-CD8a antibodies. When tumors reached to approximately 350 mm³, mice were randomized to receive subcutaneous treatment with PBS or TGFRt15-TGFRs (3 mg/kg)+TA99 (200 μg). The mice were sacrificed day 4 post-therapy and tumors were collected and analyzed. The level of GFP-positive B16F10-GFP TIS cells and NK and CD8⁺ T cells in the tumors were assess by flow cytometry. As shown in FIG. 112A, TGFRt15-TGFRs-treated mixed tumors without immunodepletion or depleted for CD8⁺ T immune cells contained significantly fewer GFP-expressing senescence tumor cells than that of control treated mice. It was also observed that the tumors of CD8⁺ depleted mice were significantly infiltrated with NK cells and tumors of NK depleted mice were significantly infiltrated with CD8⁺ T cells (FIG. 112B). These results suggested that both NK and CD8⁺ T cells play a role in controlling tumor growth with NK cells predominately mediating the activity of TGFRt15-TGFRs to deplete TIS tumor cells.

### Example 64: Anti-PD-L1 Antibody in Combination with TGFRt15-TGFRs+TA99 and Chemotherapy in B16F10 Melanoma Mouse Model

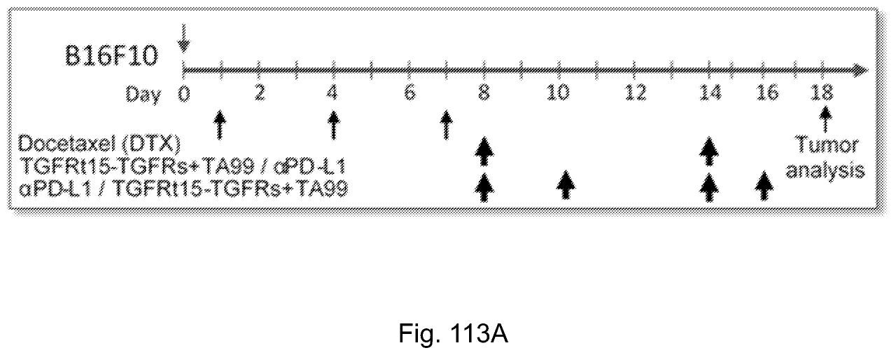

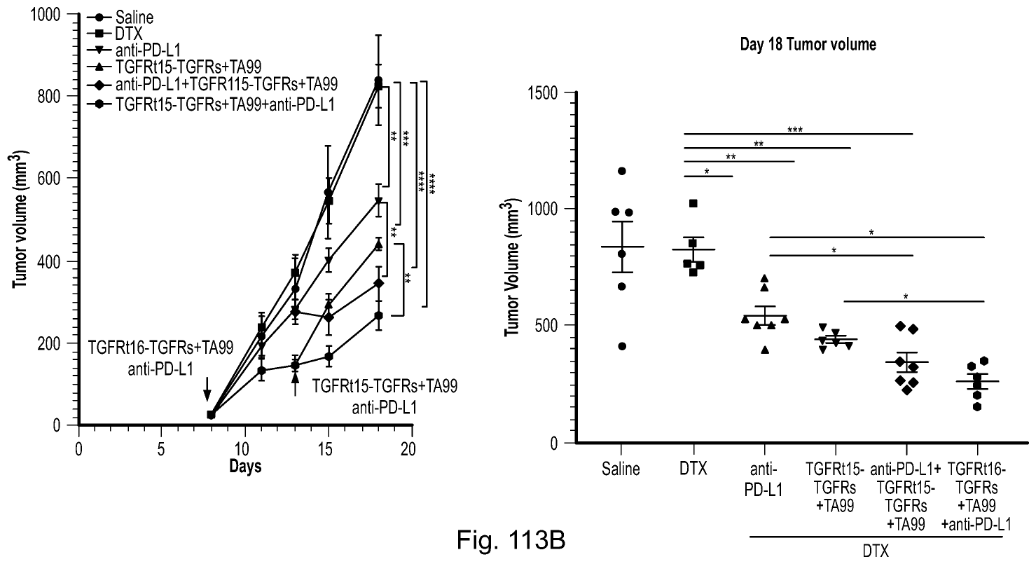

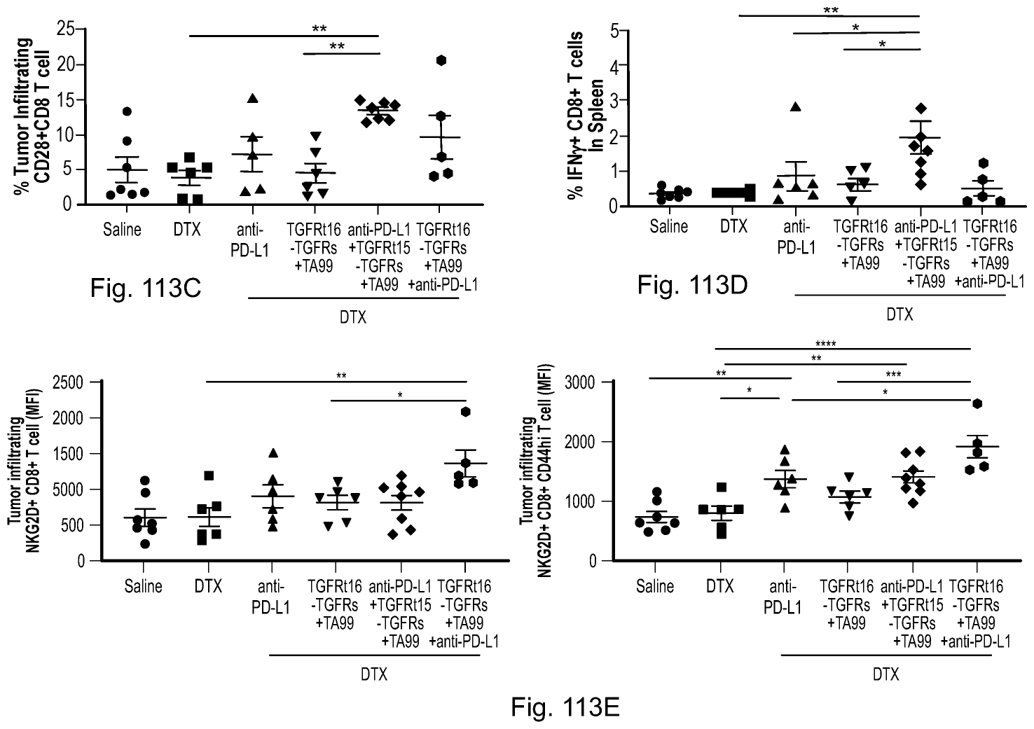

To further assess a sequential TGFRt15-TGFRs-immune checkpoint inhibitor treatment regimen (described in Example 42), B16F10 tumor-bearing mice were first treated with doxetaxel (DTX) and then either TGFRt15-TGFRs+TA99 followed by anti-PD-L1 antibody or anti-PD-L1 antibody followed by TGFRt15-TGFRs+TA99 (FIG. 113A). Tumor growth curves and end point tumor volume at day 18 indicated that both combination strategies (TGFRt15-TGFRs+TA99 followed by anti-PD-L1 and vice versa) showed significant tumor volume reduction as compared to the individual immunotherapies (either TGFRt15-TGFRs+TA99 or anti PD-L1 alone) or DTX alone (FIG. 113B). Interestingly, TGFRt15-TGFRs+TA99-treated tumors showed significantly lower tumor volume at day 13 prior to start of combination treatments as compared to anti-PD-L1-treated tumors, showing the effect of TGFRt15-TGFRs+TA99 in initial control of tumor growth. End point analysis also showed that tumors treated with the combination of TGFRt15-TGFRs+TA99 and anti-PD-L1 antibody led to significantly increased levels of tumor infiltrating CD8⁺ T cells and NK cells as compared to single treatment groups. Combination treatment increased the expression of costimulatory receptor CD28 on CD8⁺ TILs compared to single treatment suggesting that checkpoint blockade could rescue dysfunctional CD8⁺ TILs that are further activated by IL-15 activity of TGFRt15-TGFRs within the tumor microenvironment (FIG. 113C). This was concomitant with enhanced activation phenotype (IFNγ secretion) of splenic CD8⁺ T cells from combination treatment group following stimulation with PMA/ionomycin (FIG. 113D). Combination treatment also showed increased NKG2D expression on total CD8⁺ T cells and CD44^{hi} CD8⁺ T cells in the tumors compared to the individual immunotherapy treatment (FIG. 113E). These data collectively shows that combination therapy of TGFRt15-TGFRs+TA99 and anti-PD-L1 antibody led to activation and infiltration of CD8⁺ T cells that may contributed to effective tumor control.

### Example 65: Antitumor Efficacy of TGFRt15-TGFRs in Combination with Chemotherapy Against SW1990 Human Pancreatic Tumors in C57BL/6 SCID Mice

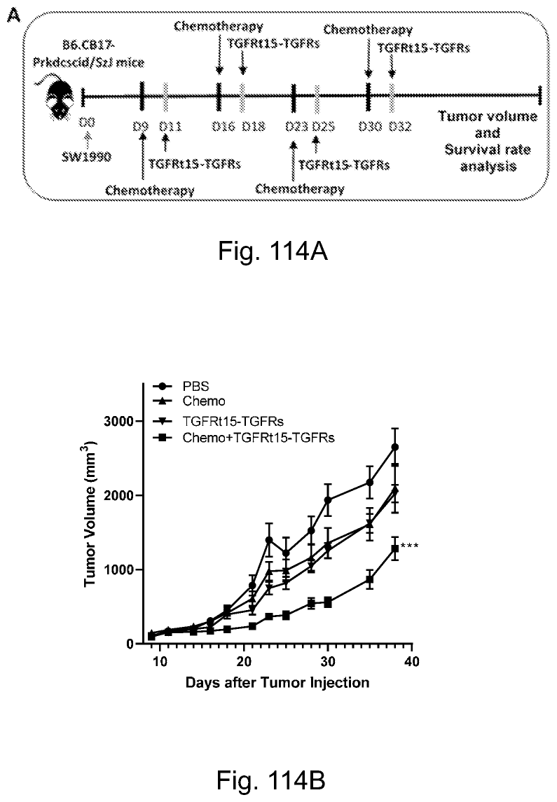

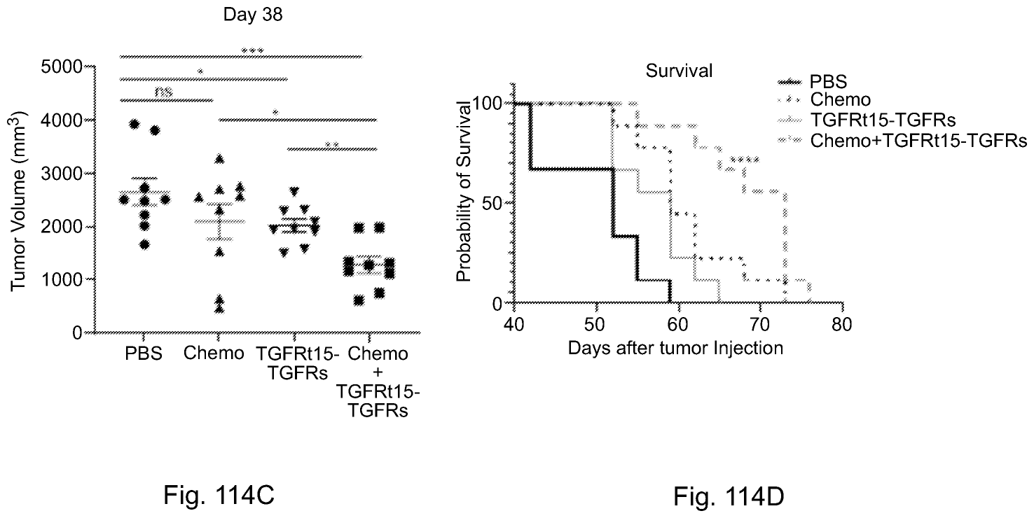

To further assess the anti-tumor activity of TGFRt15-TGFRs in combination with chemotherapy, SW1990 human pancreatic cancer cells (2×10⁶ cells/mouse) were subcutaneously (s.c.) injected into C57BL/6 scid mice. Nine days after tumor cell implantation, gemcitabine (40 mg/kg, i.p.) and nab-paclitaxel (Abraxane) (5 mg/kg, i.p.) chemotherapy was initiated followed 2 days later by TGFRt15-TGFRs (3 mg/kg, s.c.). This was considered one treatment cycle and was repeated for another 3 cycles (1 cycle/week) (FIG. 114A). Tumor-bearing control groups received PBS, chemotherapy, or TGFRt15-TGFRs treatment alone. During and after the study treatment, tumor volumes were measured and animal survival based on tumor volume<4000 mm³ was assessed. The results indicated that the animals receiving a combination of TGFRt15-TGFRs and chemotherapy had significantly slower SW1990 tumor growth comparing to the PBS group (FIG. 114B-114C). TGFRt15-TGFRs+chemotherapy also prolonged survival of SW1990 tumor-bearing mice (FIG. 114D). These results confirm that TGFRt15-TGFRs enhanced the efficacy of standard of care chemotherapy against human pancreatic tumors in a mouse xenograft tumor model.

### Example 66: Reduction in Senescent Markers in an Aged Mouse Model

C57BL/6, 72-week-old mice were purchased from the Jackson Laboratory. Mice were housed in a controlled temperature and controlled light environment. Mice were divided into five groups receiving the following treatment: Saline control (n=8), one dose of TGFRt15-TGFRs on day 0 (n=8), one dose of TGFRt15-TGFRs on day 0 followed by one dose of 2t2 on day 60 (n=7), one dose of 212 on day 0 (n=3) and one dose of 2t2 on day 0 followed by one dose of TGFRt15-TGFRs on day 60 (n=7). Mice were treated subcutaneously with PBS, TGFRt15-TGFRs (3 mg/kg), 2t2 (3 mg/kg) or TGFRt15-TGFRs (3 mg/kg) plus 2t2 (3 mg/kg). At day 120 post treatment, mice were euthanized, and livers were harvested in order to evaluate the expression levels of senescence markers IL-1α, IL6 and PAI-1 by quantitative-PCR. Harvested kidneys were stored in liquid nitrogen in 1.7 mL Eppendorf tubes. Samples were homogenized by using homogenizer in 1 mL of Trizol (Thermo Fischer). Homogenized tissues were transferred in fresh Eppendorf tubes. Total RNA was extracted using RNeasy Mini Kit (Qiagen #74106) according to the manufacturer's instructions. One μg of total RNA was used for cDNA synthesis using the QuantiTect Reverse Transcription Kit (Qiagen). Real-time PCR was carried out with CFX96 Detection System (Bio-Rad) using FAM labeled predesigned primers purchased from Thermo Scientific. Reactions were run in triplicate for all the genes examined. The housekeeping gene 18S ribosomal RNA was used as an internal control to normalize the variability in expression levels. The expression of each target mRNA relative to 18S rRNA was calculated based on Ct as 2^{-Δ(ΔCt)}, in which ΔCt=Ct_{target}−Ct_{18S}. Untreated 6-week-old mice (Young) were used as a control to compare the gene expression level to aged mice.

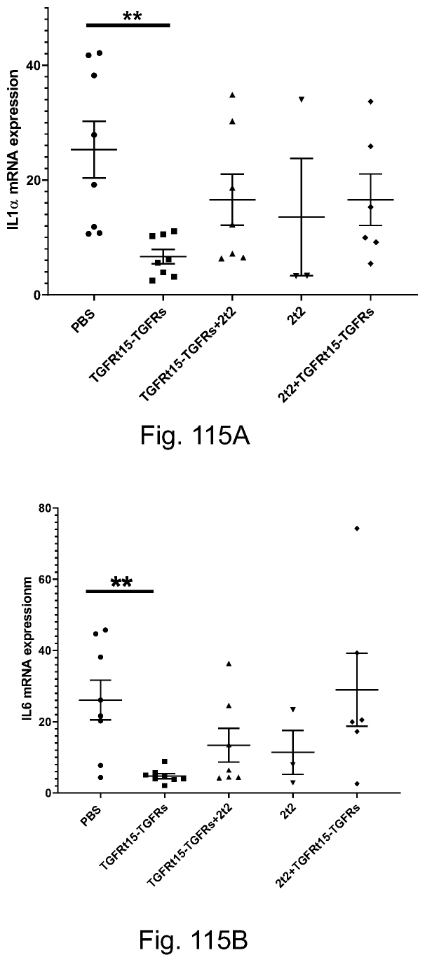

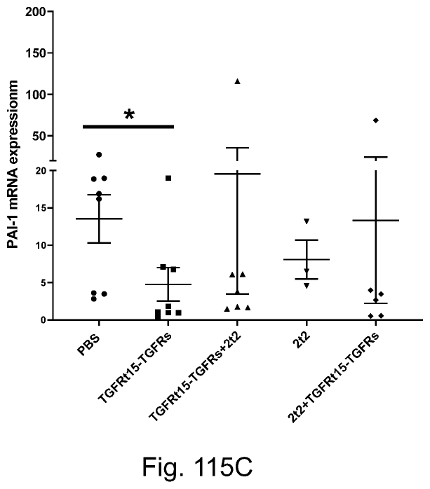

As showed in FIGS. 115A-115C, gene expression of IL-1α, IL6 and PAI-1 by in liver increased with the age of the mice as expected with the age-dependent increase in cellular senescence. Treatment of 72-month old mice with a single dose of TGFRt15-TGFRs resulted in a significant and long-lasting effect in reducing gene expression of senescence markers in livers, suggesting a treatment associated decrease in naturally-occurring senescent cells in the liver of aged mice. However, in other treatment though gene expression of IL-1α, IL6 and PAI-1 was reduced but not statically significant.

### Example 67: Treatment of Cancer

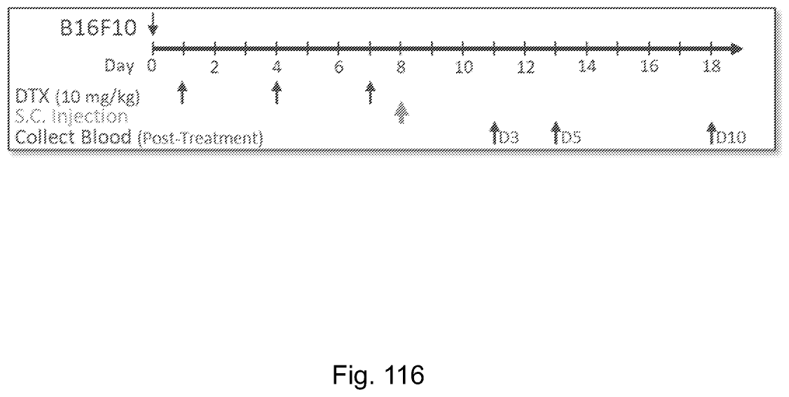

A set of experiments was performed to assess anti-tumor activity of TGFRt15-TGFRs plus anti-TRP1 antibody (TA99) in combination with chemotherapy in a melanoma mouse model. In these experiments, C57BL/6 mice were subcutaneously injected with 0.5×10⁶ B16F10 melanoma cells. The mice were treated with three doses of chemotherapy docetaxel (10 mg/kg) (DTX) on day 1, day 4, and day 7, followed by treatment with single dose of combination immunotherapy TGFRt15-TGFRs (3 mg/kg)+anti-TRP1 antibody TA99 (200 μg) on day 8. FIG. 116 shows a schematic of the treatment regimen.

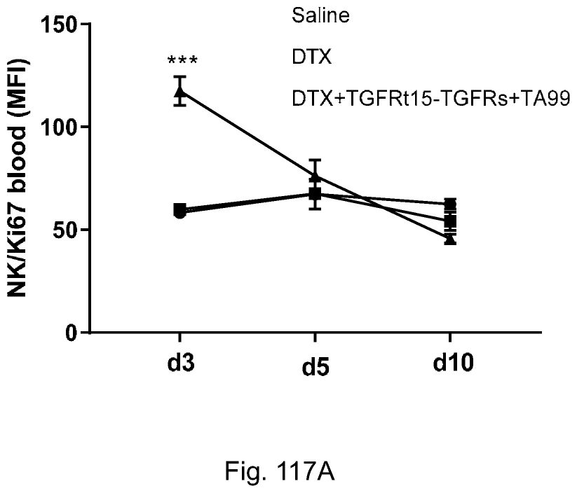

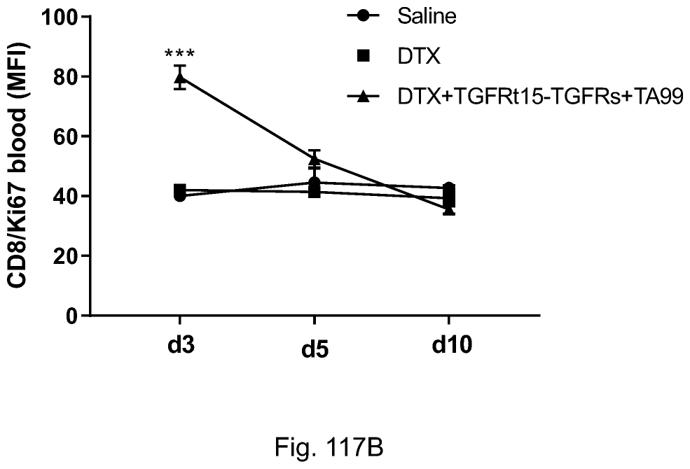

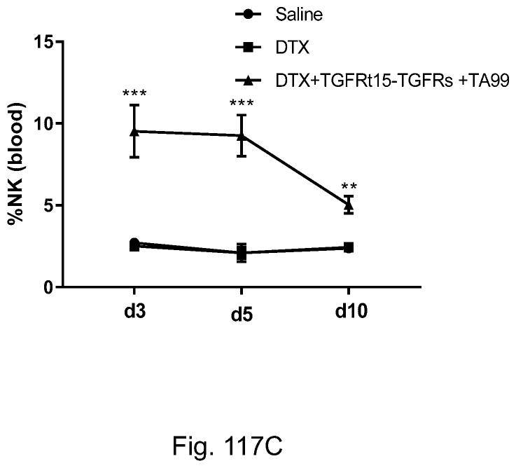

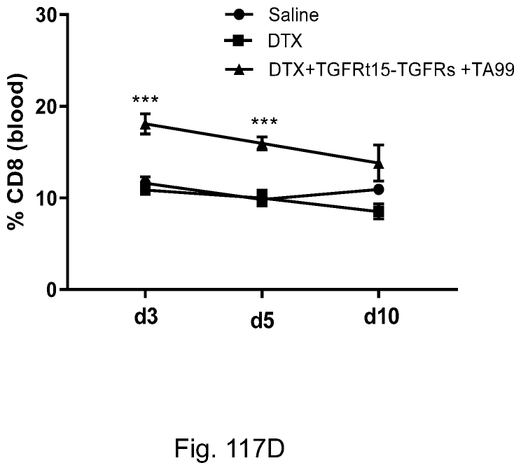

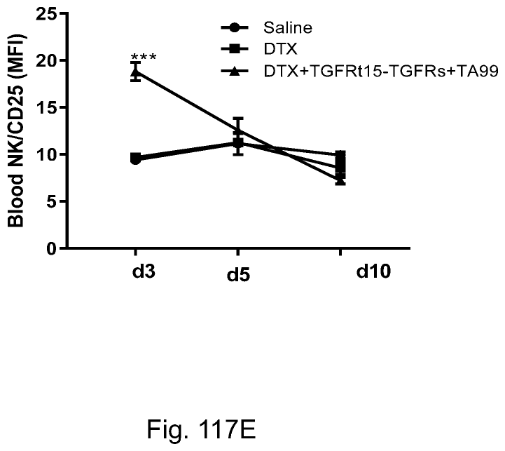

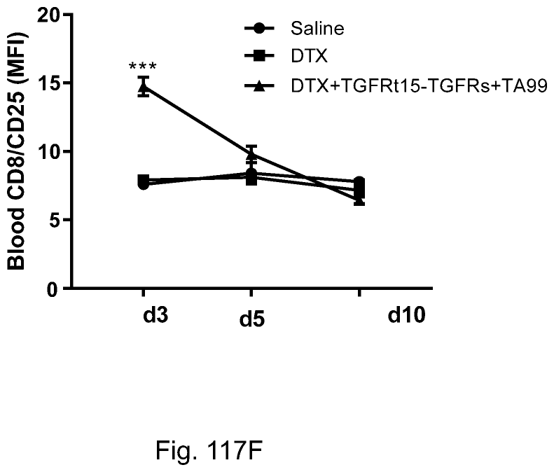

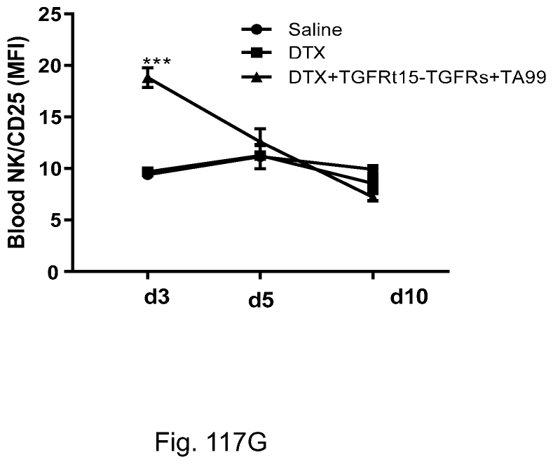

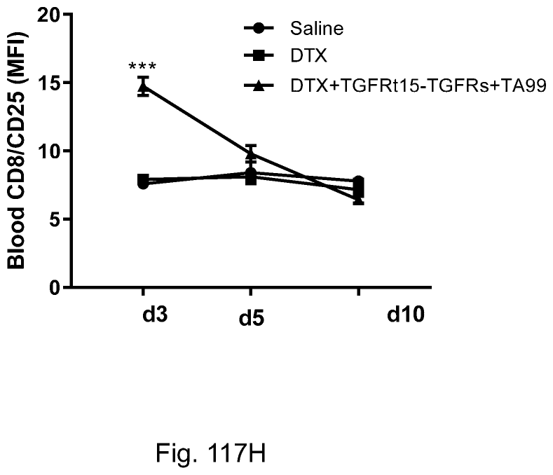

To assess immune cell subsets in the B16F10 tumor model, peripheral blood analysis was performed. In these experiments, C57BL/6 mice were injected with B16F10 cells and treated with DTX, DTX+TGFRt15-TGFRs+TA99, or saline. Blood was drawn from the submandibular vein of B16F10 tumor-bearing mice on days 3, 5, and 10 post-immunotherapy for the DTX+TGFRt15-TGFRs+TA99 group. RBCs were lysed in ACK lysis buffer and the lymphocytes were washed and stained with anti-NK1.1, anti-CD8, anti-Ki67, anti-CD25, anti-granzyme B, and anti-CD4 antibodies. The cells were analyzed by flow cytometry (Celesta-BD Bioscience). FIGS. 117A-117H show that DTX+TGFRt15-TGFRs+TA99 treatment induced an increase in the percentage of NK cells and CD8⁺ T cells in blood as compared to the saline and DTX treatment groups.

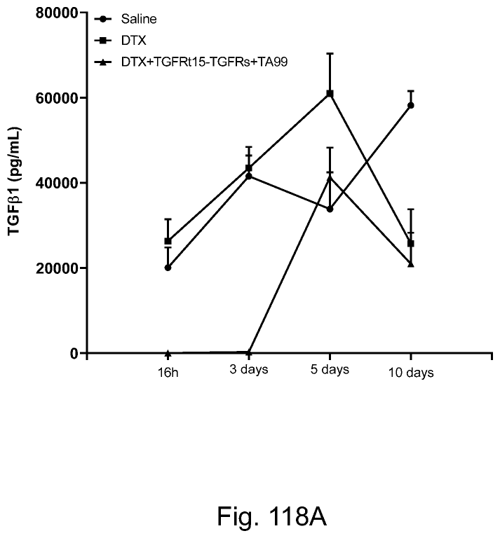

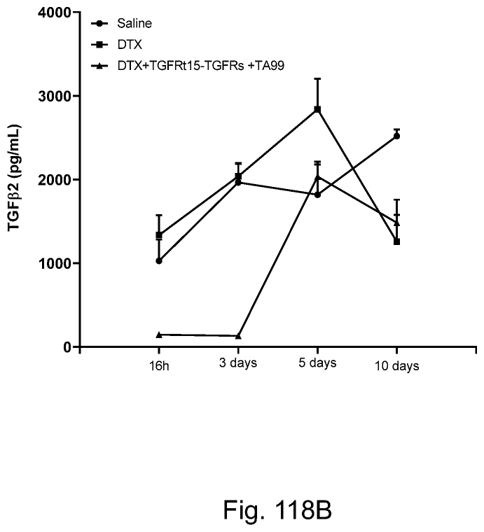

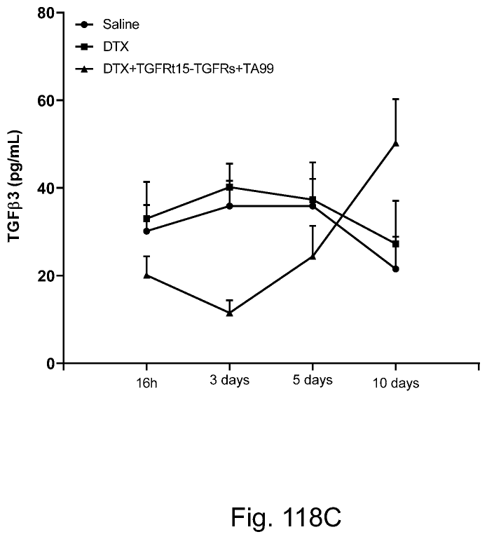

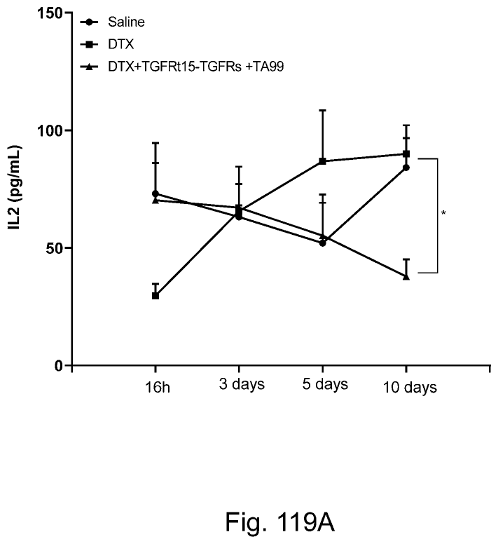

Plasma levels of TGF-β1, TGF-β2, and TGF-β3 were also determined in samples obtained at 16 hours, 3 days, 5 days, and 10 days post-immunotherapy for the DTX-TGFRt15-TGFRs+TA99 group. The data show that treatment with TGFRt15-TGFRs and TA99 reduced the plasma levels of TGF-β1 and TGF-β2 in DTX-treated mice as compared to the levels in DTX-only treated mice (FIGS. 118A-118C).

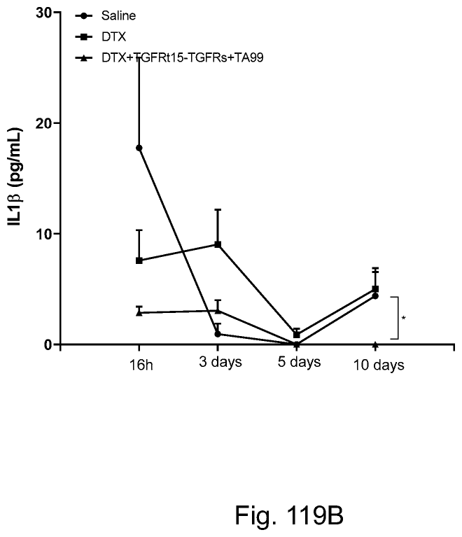

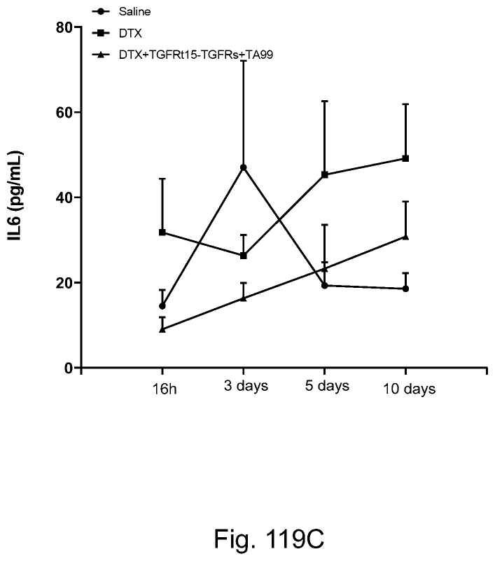

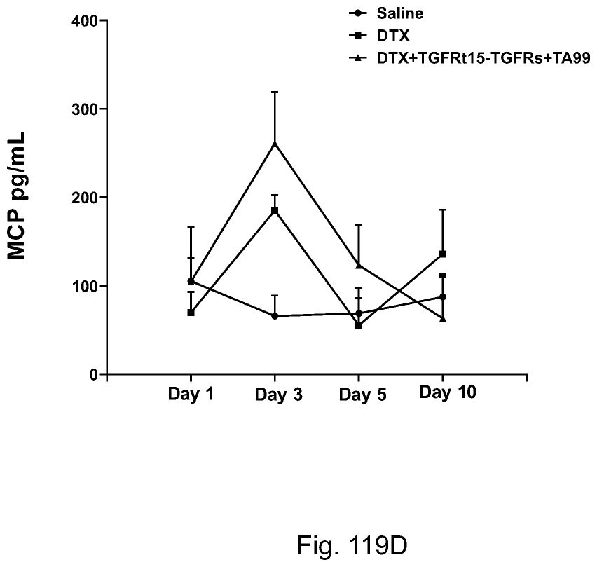

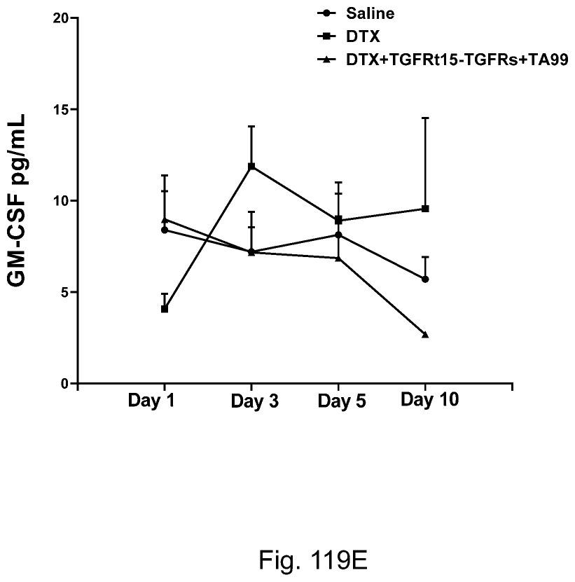

Plasma levels of IL-2, IL-1β, IL-6, MCP-1, and GM-CSF were also determined in samples obtained at 16 hours, 3 days, 5 days, and 10 days post-immunotherapy for the DTX-TGFRt15-TGFRs+TA99 group. The data show that treatment with TGFRt15-TGFRs and TA99 reduced the plasma levels of IL-2, IL-1β, IL-6, and GM-CSF in DTX-treated mice as compared to the levels in DTX-only treated mice (FIGS. 119A-119E).

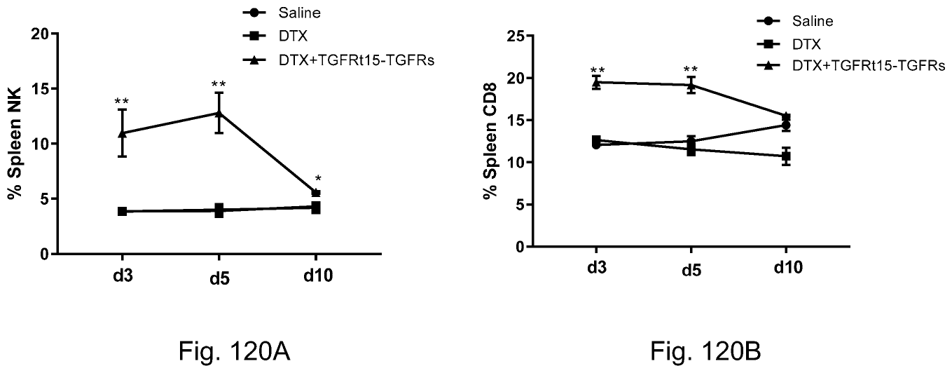

On day 18 after transplantation of B16F10 cells in the mice, the mice were sacrificed and the relative levels of NK cells and CD8⁺ T-cells in the spleens of mice were determined. The data show that treatment with TGFRt15-TGFRs and TA99 increased the NK cell and CD8⁺ T-cell levels in the spleens of DTX-treated mice, as compared to the levels in the spleens of mice treated with DTX alone (FIGS. 120A-120B).

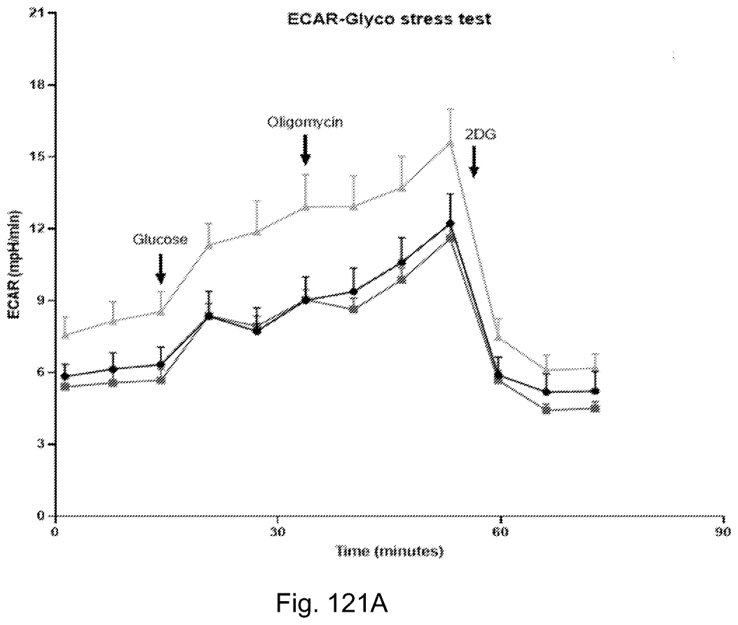

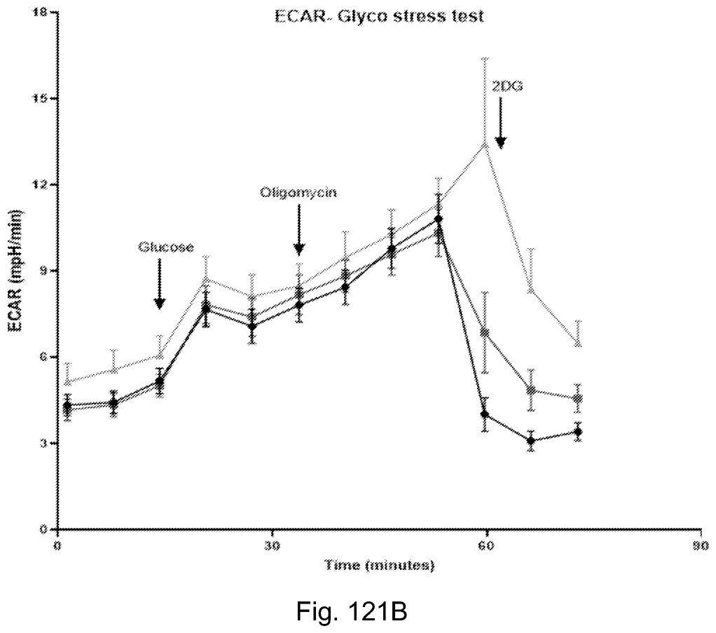

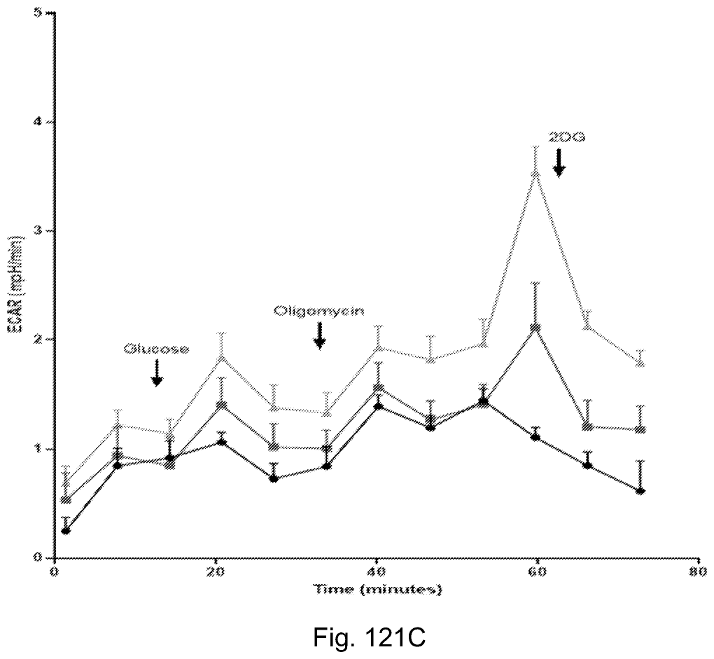

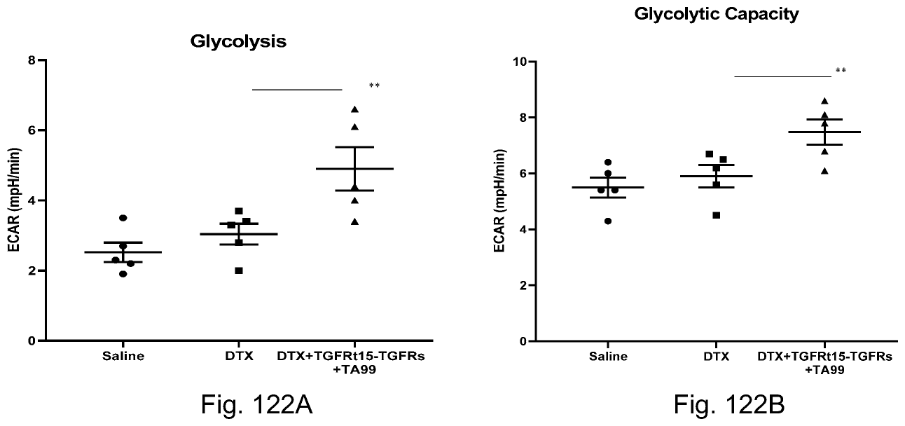

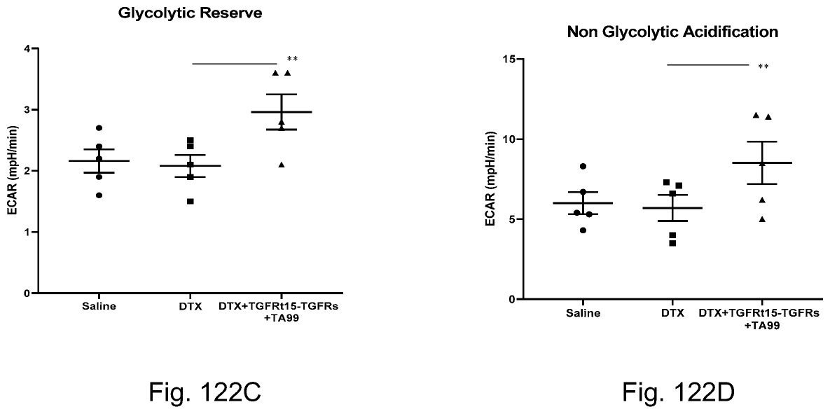

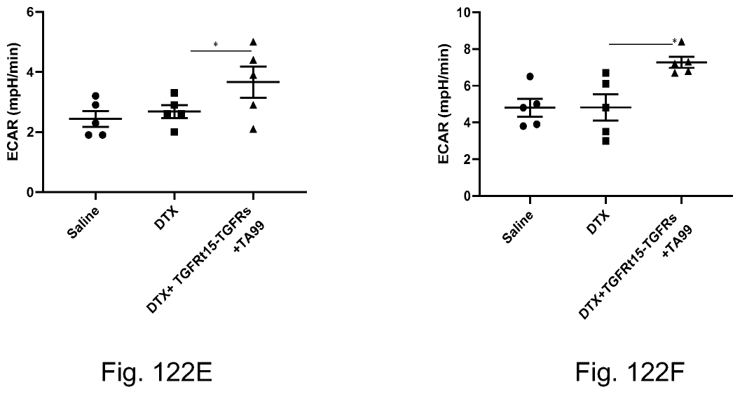

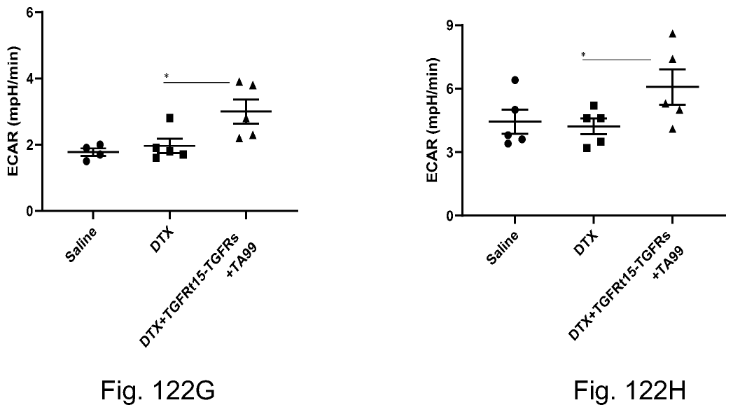

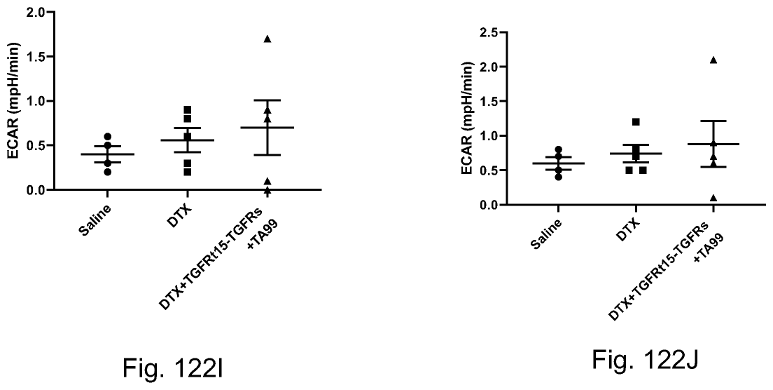

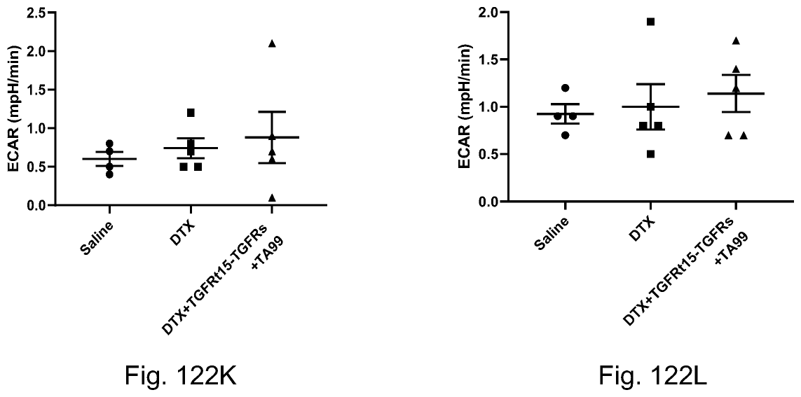

To assess glycolytic activity, glycolytic stress tests were performed in samples obtained 3 days, 5 days, and 10 days post-immunotherapy from the mice. Glycolytic activity of splenocytes from B16F10 tumor-bearing mice was determined by measuring glycolysis, glycolytic capacity, glycolytic reserve, and non-glycolytic acidification. The data show that treatment with TGFRt15-TGFRs and TA99 increased the glycolytic activity of splenocytes in DTX-treated mice as compared to the levels in DTX-only treated mice (FIGS. 121A-121C and FIGS. 122A-122L).

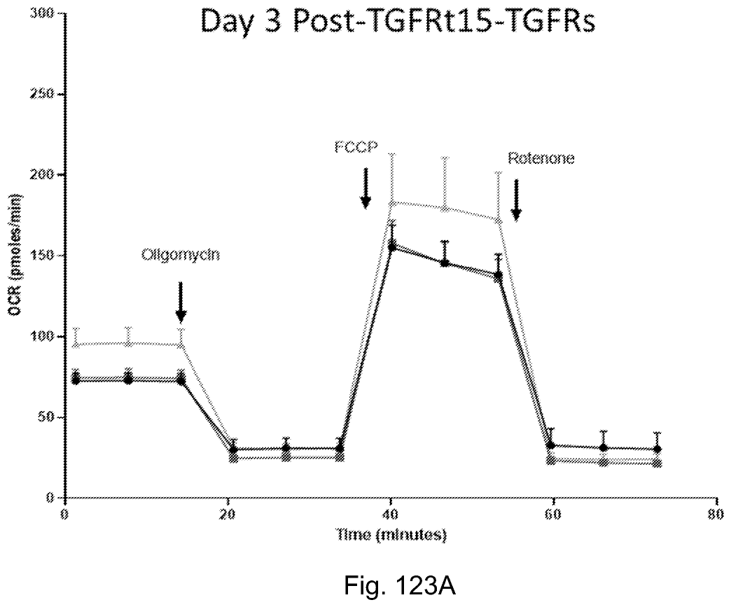

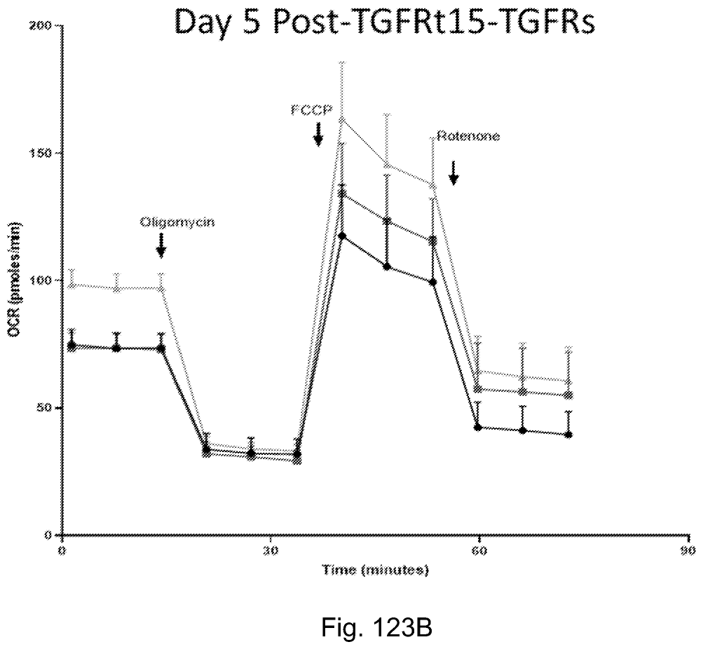

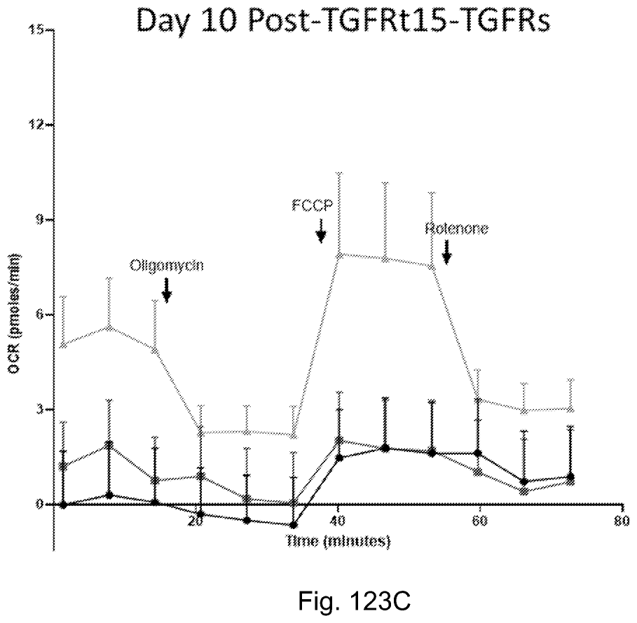

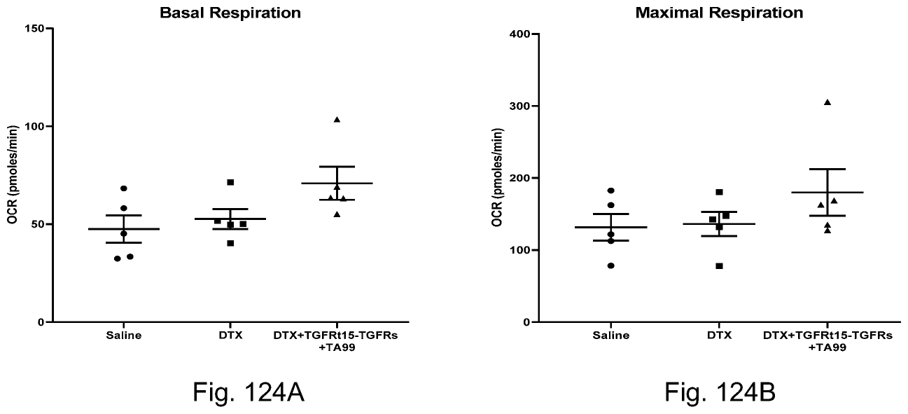

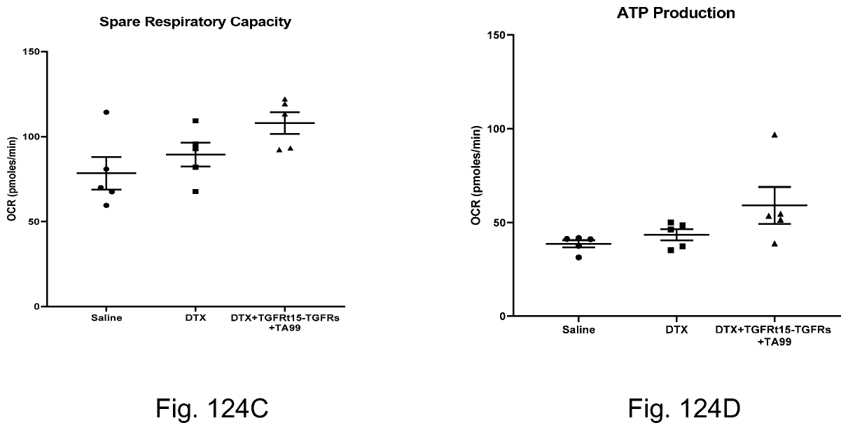

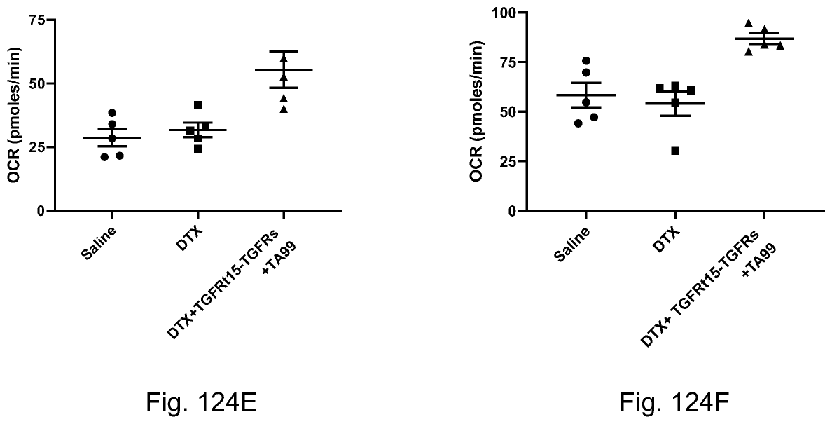

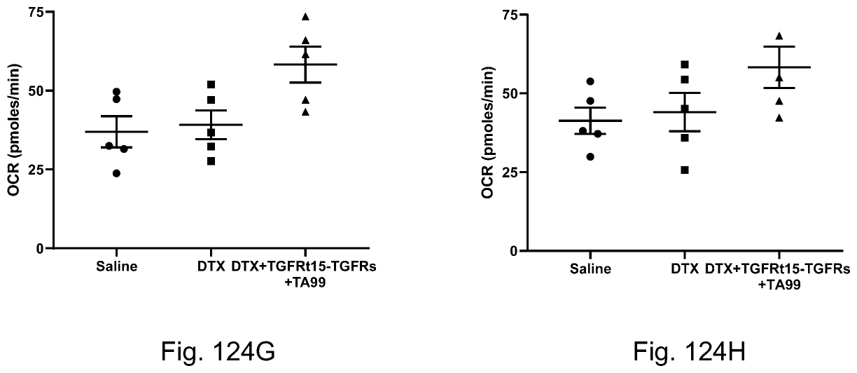

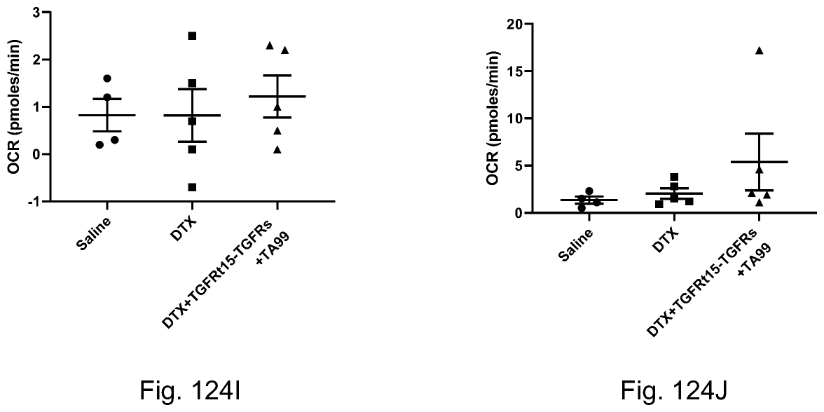

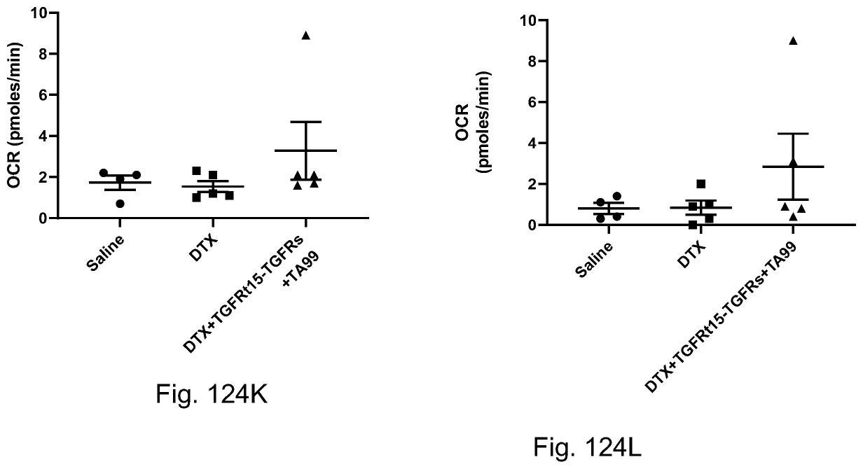

Mito stress tests were performed to further assess metabolism on splenocytes from the B16F10 tumor-bearing mice on samples obtained 3 days, 5 days, and 10 days post-immunotherapy from the mice. Mitochondrial respiration of splenocytes from the B16F10 tumor-bearing mice was also determined by measuring basal respiration, maximal respiration, spare respiratory capacity, and ATP production. The data show that treatment with TGFRt15-TGFRs and TA99 increased the mitochondrial respiration of splenocytes in DTX-treated mice as compared to the levels in DTX-only treated mice (FIGS. 123A-123C and FIGS. 124A-124L).

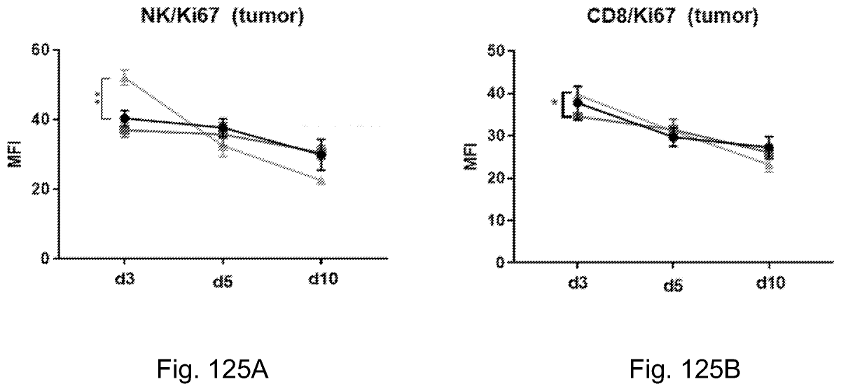

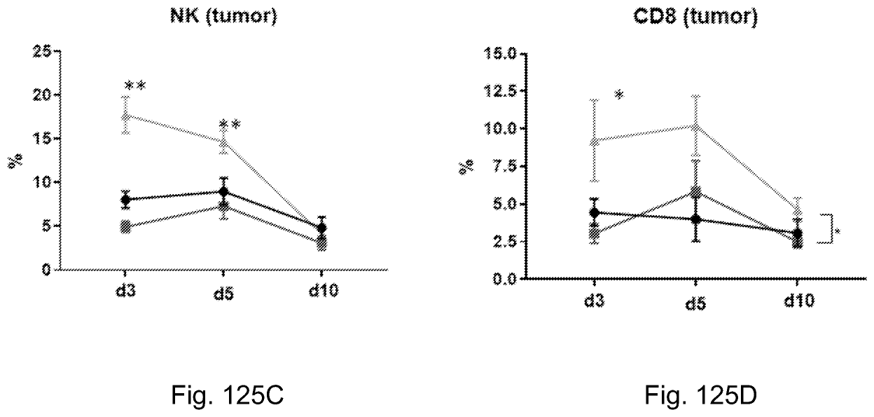

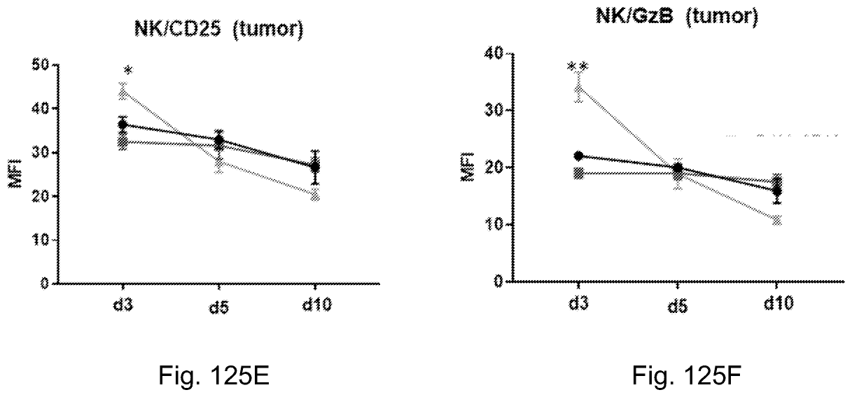

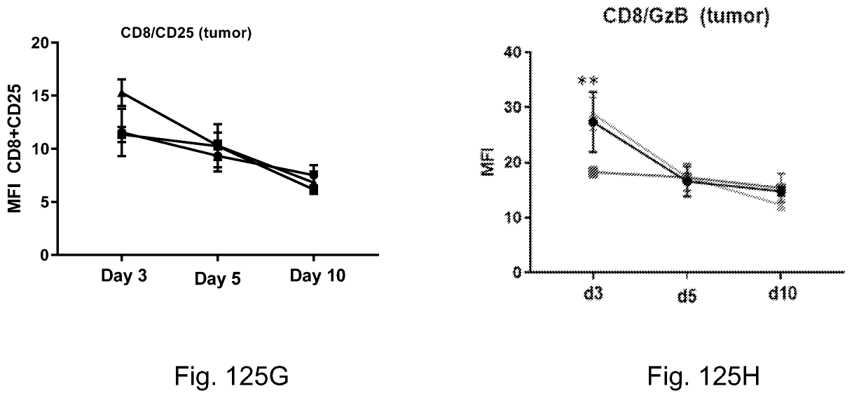

NK and T-cell tumor infiltration was also assessed in B16F10 tumors in mice treated with DTX, DTX+TGFRt15-TGFRs+TA99, or saline. FIGS. 125A-105H show that DTX+TGFRt15-TGFRs+TA99 treatment resulted in an increased level of infiltration of NK cells and CD8⁺ T cells in B16F10 tumors as compared to the saline and DTX treatment groups.

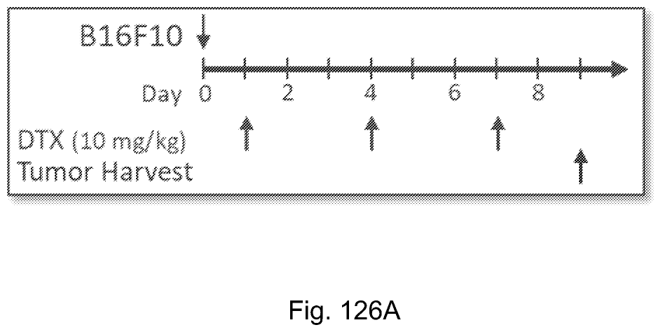

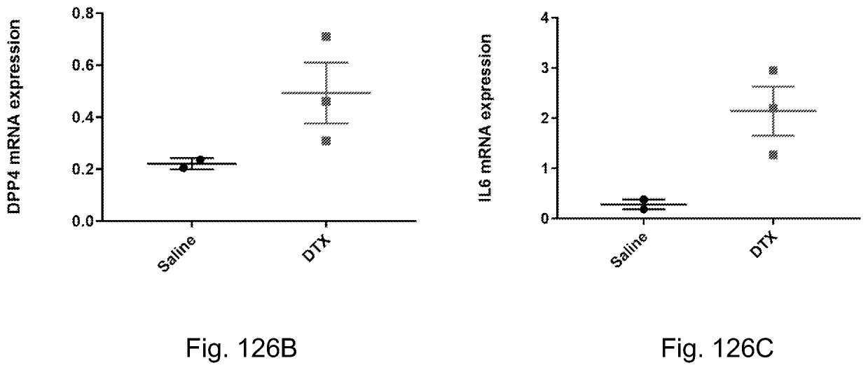

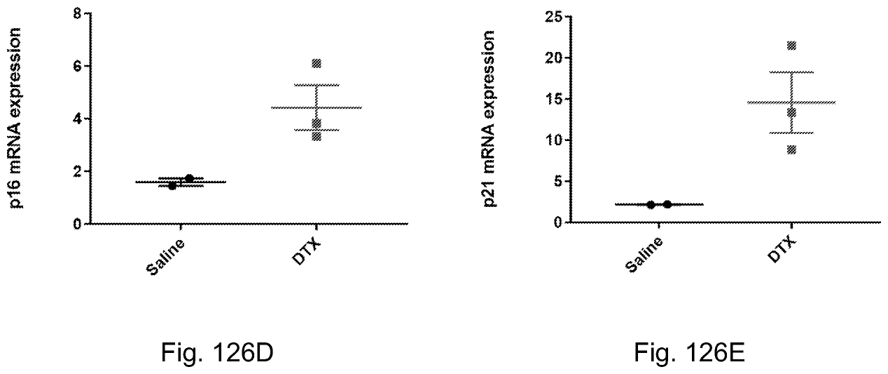

Senescence-associated gene expression in B16F10 tumors was determined in a melanoma mouse model treated with three doses of chemotherapy docetaxel (10 mg/kg) (DTX) on day 1, day 4, and day 7. FIG. 126A shows a schematic of the treatment regimen. The expression levels of DPP4, IL6, p16, and p21 in the B16F10 tumor were assessed. FIGS. 126B-126E show that DTX treatment induced an increase in senescence-associated gene expression in B16F10 tumor cells in the mice.

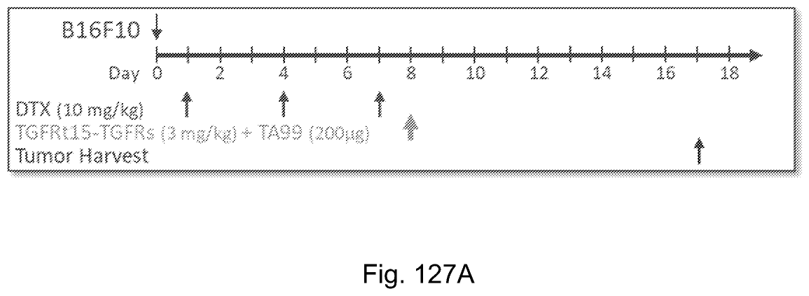

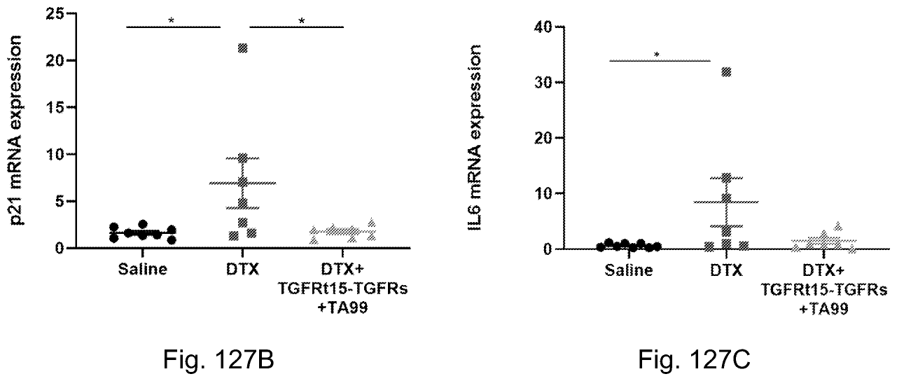

To assess the level of chemotherapy-induced senescence in B16F10 tumor cells after TGFRt15-TGFRs treatment, the mice were treated with three doses of chemotherapy docetaxel (10 mg/kg) (DTX) on day 1, day 4, and day 7 followed by a single dose of combination immunotherapy TGFRt15-TGFRs (3 mg/kg)+anti-TRP1 antibody TA99 (200 μg) on day 8. On day 17, total RNA was extracted from B16H10 tumors of mice treated with saline, DTX, or DTX+TGFRt15-TGFRs+TA99 using Trizol. FIG. 127A shows a schematic of the treatment regimen. Total RNA (1 μg) was used for cDNA synthesis using the QuantiTect Reverse Transcription Kit (Qiagen). Real-time PCR was carried out with CFX96 Detection System (Bio-Rad) using FAM-labeled predesigned primers for senescence cell markers, p21 and IL-6, the data shows that TGFRt15-TGFRs and anti-TRP1 treatment reduces p21 gene expression in B16F10 tumors in mice treated with dexamethasone (FIGS. 127B-127C).

### Example 68: Effects of TGFRt15-TGFRs and 2t2 Treatment on Mouse Plasma Markers in Aged Mice

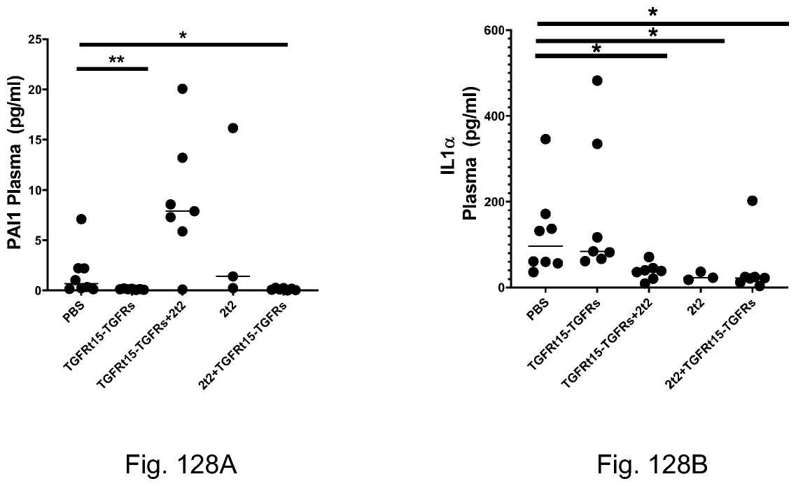

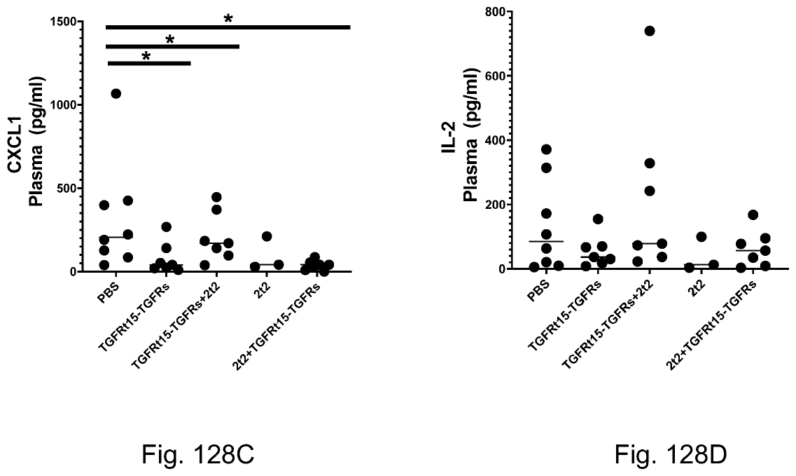

C57BL/6, 72-week-old mice were purchased from the Jackson Laboratory. Mice were housed in a controlled temperature and controlled light environment. Mice were divided into five groups receiving the following treatment: saline control (n=8), one dose of TGFRt15-TGFRs on day 0 (n=8), one dose of TGFRt15-TGFRs on day 0 followed by one dose of 2t2 on day 60 (n=7), one dose of 2t2 on day 0 (n=3) and one dose of 2t2 on day 0 followed by one dose of TGFRt15-TGFRs on day 60 (n=7). Mice were treated subcutaneously with PBS, TGFRt15-TGFRs (3 mg/kg), 2t2 (3 mg/kg) or TGFRt15-TGFRs (3 mg/kg) plus 2t2 (3 mg/kg). Mouse blood was collected from submandibular vein on day 120 in tubes containing EDTA. The whole blood was centrifuged at 3000 RPM for 10 minutes to separate plasma from blood. Plasma markers PAI-1, IL-1α and CXCL1 were analyzed by multiplex cytokine array (Eve Technologies). The results indicate that treatment of aged mice with 2t2 followed by TGFRt15-TGFRs reduced plasma levels of PAI-1, IL-1α and CXCL1 compared to control treated mice (FIGS. 128A-D). The plasma levels of IL-2 were also measured. Plasma IL-2 levels were reduced by treatment with 2t2 followed by TGFRt15-TGFRs but due to variability between animals these changes were not significant. Treatment of aged mice with TGFRt15-TGFRs alone also resulted in significant reduction in PAI-1 and CXCL1 protein levels in plasma compared to the control group (FIG. 128A-D).

### Example 69: Regulation of Transcriptomes in the Liver of db/db Mice Following Treatment with TGFRt15-TGFRs

Five-week-old male BKS.Cg-Dock7m+/+Leprdb/J (db/db) mice were fed with standard chow diet and received drinking water ad libitum. At the age of six weeks, mice were randomly assigned to control and treatment groups (n=5/group). The treatment group received TGFRt15-TGFRs by subcutaneous injection at 3 mg/kg at 6 and 12 weeks of age, while control group received vehicle (PBS) only. At end of study (4-weeks post the 2nd dose), mice were euthanized and livers were collected. The half of liver was homogenized with the TRIzol reagent (Invitrogen) and total tissue RNA was purified with RNeasy Mini Kit (Qiagen). Extracted RNA samples were quantified using Qubit 2.0 Fluorometer (Life Technologies, Carlsbad, CA, USA) and RNA integrity was checked using Agilent TapeStation 4200 (Agilent Technologies, Palo Alto, CA, USA).

RNA sequencing libraries were prepared using the NEBNext Ultra II RNA Library Prep Kit for Illumina following manufacturer's instructions (NEB, Ipswich, MA, USA). Briefly, mRNAs were first enriched with Oligo (dT) beads. Enriched mRNAs were fragmented for 15 minutes at 94° C. First strand and second strand cDNAs were subsequently synthesized. cDNA fragments were end repaired and adenylated at 3′ends, and universal adapters were ligated to cDNA fragments, followed by index addition and library enrichment by limited-cycle PCR. The sequencing libraries were validated on the Agilent TapeStation (Agilent Technologies, Palo Alto, CA, USA), and quantified by using Qubit 2.0 Fluorometer (Invitrogen, Carlsbad, CA) as well as by quantitative PCR (KAPA Biosystems, Wilmington, MA, USA).

The sequencing libraries were clustered on 1 flowcell lane. After clustering, the flowcell was loaded on the Illumina HiSeq instrument (4000 or equivalent) according to manufacturer's instructions. The samples were sequenced using a 2×150 bp Paired End (PE) configuration. Image analysis and base calling were conducted by the HiSeq Control Software (HCS). Raw sequence data (.bcl files) generated from Illumina HiSeq was converted into fastq files and de-multiplexed using Illumina's bcl2fastq 2.17 software. One mismatch was allowed for index sequence identification.

Sequence reads were trimmed to remove possible adapter sequences and nucleotides with poor quality using Trimmomatic v.0.36. The trimmed reads were mapped to the Mus musculus GRCm38 reference genome available on ENSEMBL using the STAR aligner v.2.5.2b. The STAR aligner is a splice aligner that detects splice junctions and incorporates them to help align the entire read sequences. BAM files were generated as a result of this step.

Unique gene hit counts were calculated by using featureCounts from the Subread package v.1.5.2. The hit counts were summarized and reported using the gene_id feature in the annotation file. Only unique reads that fell within exon regions were counted. If a strand-specific library preparation was performed, the reads were strand-specifically counted.

After extraction of gene hit counts, the gene hit counts table was used for downstream differential expression analysis. Using DESeq2, a comparison of gene expression between the treatment-specific groups of samples was performed. The Wald test was used to generate p-values and log 2 fold changes. Genes with an adjusted p-value<0.05 and absolute log 2 fold change>1 were called as differentially expressed genes for each comparison.

A gene ontology analysis was performed on the statistically significant set of genes by implementing the software GeneSCF v.1.1-p2. The mgi GO list was used to cluster the set of genes based on their biological processes and determine their statistical significance.

To estimate the expression levels of alternatively spliced transcripts, the splice variant hit counts were extracted from the RNA-seq reads mapped to the genome. Differentially spliced genes were identified for groups with more than one sample by testing for significant differences in read counts on exons (and junctions) of the genes using DEXSeq. For groups with only one sample, the exon hit count tables were provided.

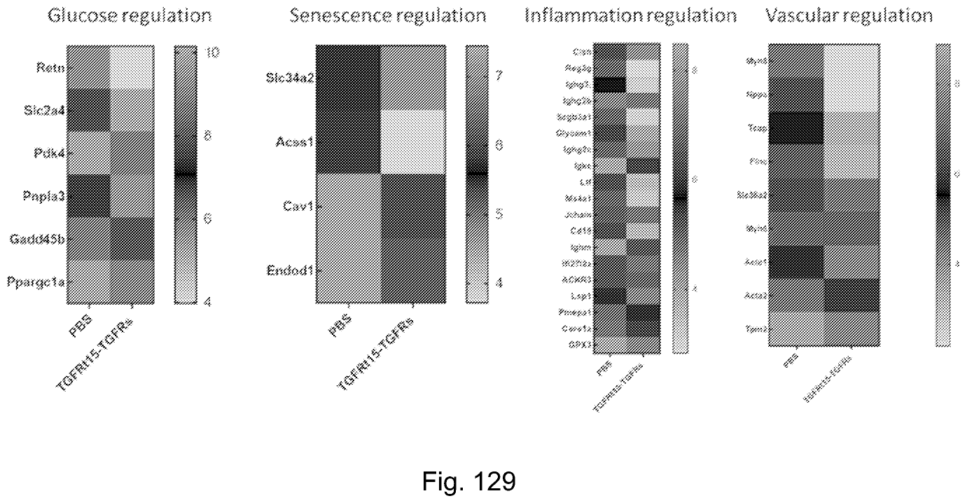

The significant genes downregulated or upregulated were divided into four groups according to the function. The heatmaps were constructed with GraphPad in accordance with gene functions. As shown in FIG. 129 and Tables 1 and 2, the six genes involved in glucose regulation were downregulated; the three genes related to senescence regulation were downregulated and one gene was upregulated; the nineteen genes involved in inflammation were mostly downregulated excepting one gene; the nine genes related to vascular regulation were downregulated.

Among six genes regulating glucose, four of them (Pdk4, Pnpla3, Gadd45b, and Ppargc1a) were related to the gluconeogenesis. Downregulation of these four genes may cause the reduction of gluconeogenesis and therefore reduce the circulating glucose. Downregulation of Retn was related to the reduction of insulin resistance. Downregulation of Slc2a4 may slow glucose transported to adipose tissue and striate muscle.

Downregulation of Cav1 and Endod1 along with upregulation of Slc34a2 promote cell proliferation and reduce senescence. Downregulation of Acss1 may reduce glucose-independent acetate-mediated cell survival and tumor growth.

Downregulation of eighteen genes and upregulation of Cish are associated with downregulation of the cells and molecules involved in inflammatory responses.

Downregulation of nine genes related to vascular regulation may reflect a different vascular environment in the liver changed by TGFRt15-TGFRs treatment.

These findings indicate that TGFRt15-TGFRs treatment suppresses gene expression related to glucose regulation, senescence, inflammation and vascular regulation in the liver of db/db mice.

TABLE 1
Regulation of transciptomes in the liver of db/db
mice following treatment with TGFRt15-TGFRs

| | | log2 Fold | | | | TGFRt15- |
|---|---|---|---|---|---|---|
| | Regulation | Change | Adj. Pval | Symbol | Control | TGFRs |
| Glucose | Down | −2.096289344 | 3.42E−02 | Retn | 5.61934746 | 3.974617084 |
| regulation | Down | −1.804543391 | 8.02E−03 | Slc2a4 | 6.648145442 | 5.116575093 |
| | Down | −1.346756214 | 9.80E−07 | Pdk4 | 10.15283618 | 8.812808986 |
| | Down | −1.319230698 | 1.82E−03 | Pnpla3 | 6.820864253 | 5.58617444 |
| | Down | −1.049951428 | 3.42E−02 | Gadd45b | 8.902818086 | 7.936747039 |
| | Down | −1.037624414 | 2.49E−03 | Ppargc1a | 9.94205237 | 8.965423502 |
| Senenscence | Up | 1.471480309 | 7.29E−02 | Slc34a2 | 5.768859731 | 7.064236232 |
| regulation | Down | −2.169290256 | 7.11E−02 | Acss1 | 5.443295282 | 3.721690488 |
| | Down | −1.610400236 | 4.18E−02 | Cav1 | 7.326547509 | 5.988279063 |
| | Down | −1.229365392 | 1.47E−02 | Endod1 | 7.446602038 | 6.192808898 |
| Inflammation | Up | 1.45707262 | 7.62E−05 | Cish | 6.321861131 | 7.704905566 |
| regulation | Down | −3.06345987 | 3.82E−02 | Reg3g | 4.932412574 | 2.829725656 |
| | Down | −2.886992871 | 8.02E−03 | Ighg3; | 5.715159646 | 3.106258191 |
| | Down | −2.826778789 | 3.00E−14 | Ighg2b | 7.437317137 | 4.709171503 |
| | Down | −2.791929301 | 3.76E−02 | Scgb3a1 | 5.023078231 | 3.024485524 |
| | Down | −2.604831835 | 8.38E−04 | Glycam1 | 6.095276793 | 3.867397849 |
| | Down | −2.562008178 | 5.67E−05 | Ighg2c | 6.725829809 | 4.258477821 |
| | Down | −2.469457569 | 1.41E−11 | Igkc | 8.457634718 | 6.060534075 |
| | Down | −2.425601648 | 9.11E−02 | Ltf | 5.274677778 | 3.477435403 |
| | Down | −2.399351243 | 4.85E−02 | Ms4a1 | 5.017374783 | 3.304853422 |
| | Down | −2.047374161 | 8.63E−05 | Jchain | 6.660474662 | 4.797517709 |
| | Down | −2.039004438 | 7.47E−02 | Cd19 | 5.303977874 | 3.593322805 |
| | Down | −2.036901274 | 3.47E−07 | Ighm | 8.299180551 | 6.411154088 |
| | Down | −1.909054054 | 1.65E−03 | Ifi27l2a | 6.420560573 | 4.753540666 |
| | Down | −1.706656106 | 4.71E−03 | ACKR3 | 6.465216788 | 4.982147331 |
| | Down | −1.467633284 | 3.97E−02 | Lsp1 | 5.884533344 | 4.544074275 |
| | Down | −1.11979923 | 4.71E−03 | Pmepa1 | 6.90863891 | 5.841136195 |
| | Down | −1.046058196 | 7.11E−02 | Coro1a | 7.076869734 | 6.124311808 |
| | Down | −1.038663712 | 2.19E−02 | GPX3 | 8.488888196 | 7.517625851 |
| Vascular | Down | −5.614042335 | 2.87E−02 | Myh8 | 4.469324747 | 2.214290342 |
| regulation | Down | −5.507281406 | 1.65E−03 | Nppa | 5.06772092 | 2.19472816 |
| | Down | −3.436742187 | 2.65E−03 | Tcap | 5.595187029 | 3.260187895 |
| | Down | −2.764903666 | 9.49E−02 | [symbol] lnc | 4.862671393 | 3.129487218 |
| | Down | −2.518127305 | 8.16E−03 | Slc36a2 | 6.244224409 | 4.402606708 |
| | Down | −2.317372389 | 3.00E−10 | Myh6 | 6.928245867 | 4.797511367 |
| | Down | −2.093059568 | 2.02E−02 | Actc1 | 5.784720054 | 4.123883209 |
| | Down | −1.550587332 | 2.25E−02 | Acta2 | 7.25724117 | 5.925475021 |
| | Down | −1.281068362 | 1.07E−03 | Tpm2 | 8.878431842 | 7.68227915 |

[symbol]

indicates data missing or illegible when filed

TABLE 2
Regulation of transcriptomes in the liver of db/db mice following treatment with TGFRt15-TGFRs

| [symbol] | [symbol] | [symbol] | [symbol] | [symbol] | [symbol] | [symbol] | [symbol] | [symbol] |
|---|---|---|---|---|---|---|---|---|
| [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] |
| Senenscent cell | Up | ENSMUSG00000029188 | 1.471480309 | 7.29E−02 | Slc34a2 | 5.282818592 | 6.24485242 | 5.77890818 |
| regulation | Down | ENSMUSG00000007655 | −1.610400236 | 4.18E−02 | Cav1 | 7.962951204 | 7.996804652 | 6.019886672 |
| | Down | ENSMUSG00000037419 | 1.229365392 | 1.47E−02 | Endod1 | 7.497030385 | 7.216009636 | 7.626766094 |
| Inflammation | Up | ENSMUSG00000032578 | 1.45707262 | 7.62E−05 | Cish | 6.611948282 | 6.206382894 | 6.147252218 |
| regulation | Down | ENSMUSG00000030017 | −3.06345987 | 3.82E−02 | Reg3g | 5.167366003 | 5.724919575 | 3.904952145 |
| | Down | ENSMUSG00000076615 | 2.886992871 | 8.02E−03 | Ighg3; | 6.041689587 | 5.551619277 | 5.552170073 |
| | Down | ENSMUSG00000076613 | −2.826778789 | 3.00E−14 | Ighg2b | 7.58214335 | 7.456259351 | 7.273548711 |
| | Down | ENSMUSG00000064057 | 2.791929301 | 3.76E−02 | Scgb3a1 | 5.489230064 | 5.581987166 | 3.998017464 |
| | Down | ENSMUSG00000022491 | −2.604831835 | 8.38E−04 | Glycam1 | 6.536403573 | 6.581851777 | 5.16757503 |
| | Down | ENSMUSG00000076612 | −2.562008178 | 5.67E−05 | Ighg2c | 6.904107468 | 7.12612974 | 6.147252218 |
| | Down | ENSMUSG00000076609 | −2.469457569 | 1.41E−11 | Igkc | 8.738614728 | 8.835986045 | 7.798303381 |
| | Down | ENSMUSG00000032496 | 2.425601648 | 9.11E−02 | Ltf | 5.75222178 | 6.166859409 | 3.904952145 |
| | Down | ENSMUSG00000024673 | −2.399351243 | 4.85E−02 | Ms4a1 | 5.354953962 | 5.611728983 | 4.085441405 |
| | Down | ENSMUSG00000067149 | −2.047374161 | 8.63E−05 | Jchain | 6.85520118 | 7.084312295 | 6.041910512 |
| | Down | ENSMUSG00000030724 | −2.039004438 | 7.47E−02 | Cd19 | 5.75222178 | 5.354622897 | 4.805088944 |
| | Down | ENSMUSG00000076617 | −2.036901274 | 3.47E−07 | Ighm | 8.69063561 | 8.774860615 | 7.432045428 |
| | Down | ENSMUSG00000079017 | −1.909054054 | 1.65E−03 | Ifi2 [symbol] 2a | 6.765410892 | 6.854846927 | 5.6414239 |
| | Down | ENSMUSG00000044337 | −1.706656106 | 4.71E−03 | ACKR3 | 5.904203528 | 6.354464397 | 7.136982438 |
| | Down | ENSMUSG00000018819 | −1.467633284 | 3.97E−02 | Lsp1 | 5.904203528 | 5.996956972 | 5.752439532 |
| | Down | ENSMUSG00000038400 | −1.11979923 | 4.71E−03 | Pmepa1 | 6.87986155 | 6.804224856 | 7.041830324 |
| | Down | ENSMUSG00000030707 | −1.046058196 | 7.11E−02 | Coro1a | 7.319120952 | 7.406221275 | 6.505266974 |
| | Down | ENSMUSG00000018339 | −1.038663712 | 2.19E−02 | GPX3 | 8.731857489 | 8.879424465 | 7.855382635 |
| Vascular | Down | ENSMUSG00000055775 | −5.614042335 | 2.87E−02 | Myh8 | 5.456814408 | 5.951159834 | 2 |
| regulation | Down | ENSMUSG00000041616 | −5.507281406 | 1.65E−03 | Nppa | 4.641438054 | 5.206558526 | 5.355166181 |
| | Down | ENSMUSG00000007877 | −3.436742187 | 2.65E−03 | Tcap | 6.505042126 | 6.69731858 | 3.583200381 |
| | Down | ENSMUSG00000068699 | −2.764903666 | 9.49E−02 | Flnc | 5.641207544 | 5.778349917 | 3.168456718 |
| | Down | ENSMUSG00000020264 | −2.518127305 | 8.16E−03 | Slc36a2 | 7.126486166 | 7.216009636 | 4.390177426 |
| | Down | ENSMUSG00000040752 | −2.317372389 | 3.00E−10 | Myh6 | 6.985896265 | 7.186667466 | 6.612173869 |
| | Down | ENSMUSG00000068614 | −2.093059568 | 2.02E−02 | Actc1 | 6.319192998 | 6.336764352 | 4.698202813 |
| | Down | ENSMUSG00000035783 | −1.550587332 | 2.25E−02 | Acta2 | 7.380894129 | 8.089558631 | 6.30127075 |
| | Down | ENSMUSG00000028464 | −1.281068362 | 1.07E−03 | Tpm2 | 9.256882877 | 9.21109488 | 8.167317769 |

| [symbol] | [symbol] | [symbol] | [symbol] | [symbol] | [symbol] | [symbol] | [symbol] |
|---|---|---|---|---|---|---|---|
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| Senenscent cell | Up | ENSMUSG00000029188 | 5.768859731 | 6.805137717 | 7.980405385 | 6.407165595 | 7.064236232 |
| regulation | Down | ENSMUSG00000007655 | 7.326547509 | 6.390127612 | 5.90443626 | 5.670273318 | 5.988279063 |
| | Down | ENSMUSG00000037419 | 7.446602038 | 6.843270702 | 5.830444913 | 5.904711078 | 6.192808898 |
| Inflammation | Up | ENSMUSG00000032578 | 6.321861131 | 7.886482699 | 8.030833382 | 7.197400617 | 7.704905566 |
| regulation | Down | ENSMUSG00000030017 | 4.932412574 | 3.320548901 | 2 | 3.168628066 | 2.829725656 |
| | Down | ENSMUSG00000076615 | 5.715159646 | 4.320088877 | 2.998685697 | 2 | 3.106258191 |
| | Down | ENSMUSG00000076613 | 7.437317137 | 4.457550573 | 5.423879785 | 4.246084151 | 4.709171503 |
| | Down | ENSMUSG00000064057 | 5.023078231 | 3.168648016 | 2.321402517 | 3.583406038 | 3.024485524 |
| | Down | ENSMUSG00000022491 | 6.095276793 | 4.583046598 | 3.698619594 | 3.320527354 | 3.867397849 |
| | Down | ENSMUSG00000076612 | 6.725829809 | 4.521662724 | 5.085142674 | 3.168628066 | 4.258477821 |
| | Down | ENSMUSG00000076609 | 8.457634718 | 6.187651259 | 6.536642214 | 5.457308753 | 6.060534075 |
| | Down | ENSMUSG00000032496 | 5.274677778 | 3.457968795 | 2.80622845 | 4.168108965 | 3.477435403 |
| | Down | ENSMUSG00000024673 | 5.017374783 | 3.457968795 | 3.457758661 | 2.998832811 | 3.304853422 |
| | Down | ENSMUSG00000067149 | 6.660474662 | 5.167881281 | 4.904611708 | 4.320060138 | 4.797517709 |
| | Down | ENSMUSG00000030724 | 5.303977874 | 4.168136905 | 3.805476979 | 2.806354532 | 3.593322805 |
| | Down | ENSMUSG00000076617 | 8.299180551 | 6.670216194 | 6.456921454 | 6.106324617 | 6.411154088 |
| | Down | ENSMUSG00000079017 | 6.420560573 | 4.997988235 | 4.457280316 | 4.805353448 | 4.753540666 |
| | Down | ENSMUSG00000044337 | 6.465216788 | 4.952193823 | 4.951906104 | 5.042342067 | 4.982147331 |
| | Down | ENSMUSG00000018819 | 5.884533344 | 4.904898004 | 4.641647476 | 4.085677346 | 4.544074275 |
| | Down | ENSMUSG00000038400 | 6.90863891 | 5.805219897 | 5.698012376 | 6.020176311 | 5.841136195 |
| | Down | ENSMUSG00000030707 | 7.076869734 | 6.042234192 | 6.245436239 | 6.085264994 | 6.124311808 |
| | Down | ENSMUSG00000018339 | 8.488888196 | 7.711989961 | 7.23584466 | 7.605042932 | 7.517625851 |
| Vascular | Down | ENSMUSG00000055775 | 4.469324747 | 2.32146851 | 2.321402517 | 2 | 2.214290342 |
| regulation | Down | ENSMUSG00000041616 | 5.06772092 | 2 | 2 | 2.584184479 | 2.19472816 |
| | Down | ENSMUSG00000007877 | 5.595187029 | 3.168648016 | 2.80622845 | 3.80568722 | 3.260187895 |
| | Down | ENSMUSG00000068699 | 4.862671393 | 2.806369918 | 2.998685697 | 3.583406038 | 3.129487218 |
| | Down | ENSMUSG00000020264 | 6.244224409 | 4.246112511 | 4.641647476 | 4.320060138 | 4.402606708 |
| | Down | ENSMUSG00000040752 | 6.928245867 | 4.997988235 | 4.641647476 | 4.752898391 | 4.797511367 |
| | Down | ENSMUSG00000068614 | 5.784720054 | 4.246112511 | 3.805476979 | 4.320060138 | 4.123883209 |
| | Down | ENSMUSG00000035783 | 7.25724117 | 6.245749236 | 5.725489293 | 5.805186533 | 5.925475021 |
| | Down | ENSMUSG00000028464 | 8.878431842 | 7.663081872 | 7.473162775 | 7.910592804 | 7.68227915 |

| [symbol] | [symbol] | [symbol] | [symbol] |
|---|---|---|---|
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| Senenscent cell | Up | ENSMUSG00000029188 | Solute carrier family 34 m3mber 2, (SLC34A2), a member of the |
| regulation | | | S [symbol] C34 family, was initially isolated from a human small |
| | | | intestine. SLC34A2 is a multipass membrane protein and |
| | | | encodes a type 2b sodium-dependent phosphate transporter, |
| | | | NaPiIIb. It is known that SLC34A2 can mediate transporting |
| | | | inorganic phosphate into epithelial cells via sodium ion |
| | | | cotransport. Knockdown of SLC34A2 inhibits proliferation and |
| | | | migration by suppressing activation of the PI3K/AKT signaling |
| | | | pathway in hepatocellular carcinoma cells (HCC). |
| | Down | ENSMUSG00000007655 | Caveolin-1 is a scaffolding protein as the main component of the |
| | | | caveolae plasma membranes found in most cell types. The |
| | | | protein links integrin to promote cell cycle progression. |
| | | | Caveolin-1 plays a central role in the deveopment of a |
| | | | senescent phenotype and the regulation of both the anti |
| | | | tumorigenicand proterties of cellular senescence. Caveolin-1 in |
| | | | expression controls on specific TGF-?1/p53 responsive growth |
| | | | arrest genes. Indeed, up-regulation of caveolin 1 appears to |
| | | | stall cells in G0/G1 via activation of the p53/p21 cell cycle arrest |
| | | | pathway. The liver expresses deteactable CAV1 levels and, |
| | | | although some curvature has been described in the sinusoidal |
| | | | plasma membranes, hepatocytes do not form abundant |
| | Down | ENSMUSG00000037419 | Endonuclease domain containing 1 is a novel tumor suppressor |
| | | | in prostate cancer. Endonuclease domain containing 1 |
| | | | (ENDOD1) is a member of nucleases, which hydrolyze |
| | | | phosphodiester linkage in nucleic acids. It has been reported |
| | | | that nucleases participate in mutation avoidance, DNA repair |
| Inflammation | Up | ENSMUSG00000032578 | Cytokine-inducible SH2-containing protein negatively regulates |
| regulation | | | TCR signaling |
| | Down | ENSMUSG00000030017 | Regenerating islet-derived protein 3 gamma is one of several |
| | | | antimicrobial peptides. Among Reg family genes, Reg III? and |
| | | | III? were alternatively overexpressed in the colonic tissues of |
| | | | mice with DSS-induced colitis. The expression of STAT3- |
| | | | associated cytokines (IL-6, IL-17, and IL-22) was also significantly |
| | | | increased in those tissues, being significantly correlated with |
| | | | that o [symbol] Reg III?/?. In the normal pancreas, Reg3? staining is |
| | | | absent or very minimally observed as small focal areas of |
| | Down | ENSMUSG00000076615 | |
| | Down | ENSMUSG00000076613 | |
| | Down | ENSMUSG00000064057 | Secretoglobin family 3 A member 1, SCGB3A1, also called UGRP2 |
| | | | or high in normal-1 (HIN 1), was described as a tumor |
| | | | suppressor in various human tumors including breast, prostate, |
| | | | lung, and pancreatic carcinomas. Ugrp2 gene is localized at |
| | | | chromosome 11B1 [3], a homologous region to 5q31 q35 in |
| | | | human, in which many genes encoding inflammatory cytokines |
| | | | such as interleukin-3, -4, -5, -13 and colony-stimulating factor 2 |
| | | | are located. These facts together with the sites of UGRP2 |
| | Down | ENSMUSG00000022491 | GlyCAM1 (Glycosylation-dependent cell adhesion molecule 1) is |
| | | | a proteoglycan ligand for [symbol] -selectin, modulating |
| | | | transendothelial migration of leukocytes. Existing evidence |
| | | | supports a role for GlyCAM1 as a negative regulator of |
| | | | extravasation. GlyCAM1 levels (protein and mRNA) decrease |
| | | | during acute antigen-primed inflammation and depletion of |
| | | | soluble L-selectin ligands (using L-selectin-IgG affinity columns) |
| | Down | ENSMUSG00000076612 | |
| | Down | ENSMUSG00000076609 | |
| | Down | ENSMUSG00000032496 | Lactoferrin (Lf) is a conserved iron binding glycoprotein with |
| | | | antimicrobial activity. The infiltration of neutrophils into |
| | | | intestine tissues was changed similarly to Lf expression. It |
| | | | indicated that the variations of Lf expression were rather due to |
| | Down | ENSMUSG00000024673 | Membrane spanning 4-domain A1 encodes CD20. |
| | Down | ENSMUSG00000067149 | |
| | Down | ENSMUSG00000030724 | CD19 plays an essential role in regulating B-cell activation |
| | | | thresholds and thereby influences B cell selection and |
| | | | differentiation. Altering CD19 surface expression in knockout or |
| | | | transgenic mice significantly changes B-cell development and |
| | | | function. CD19 overexpression results in B cells that are hyper- |
| | | | responsive to BCR triggering, leading to a lupus-like |
| | | | autoimmune disease with the production of anti-nuclear |
| | | | antibodies (ANAs) in the serum of transgenic mice (7). The |
| | Down | ENSMUSG00000076617 | |
| | Down | ENSMUSG00000079017 | Interferon alpha-inducible protein 27 like 2a is strongly up- |
| | | | regulated in the lung after influenza A infection. |
| | Down | ENSMUSG00000044337 | Atypical chemokine receptor 3 also known as CXCR-7 and |
| | | | GPR159 can bind the chemokines CXCL 12/SDF-1 and CXCL-11. |
| | | | ACKR3 functions primarily by sequestering the chemokine CXC- |
| | | | 12 and endogenous opioid peptides. ACKR3 expression is |
| | | | usually faint or undetectable at steady state in the endothelium |
| | | | and in myeloid cells, but it can be upregulated during |
| | | | inflammation, for instance, by proinflammatory cytokines, such |
| | | | as interleukin8 (Singh and Lokeshwar, 2011) or IL-1b in vitro |
| | | | (Watanabe et al., 2010) and by environmental cues, such as |
| | | | lipopolysaccharide (Cao et al., 2016; Konrad et al., 2017; Ngamsri |
| | | | et al., 2017) or during infection by oncoviruses [reviewed in |
| | | | Freitas et al. (2014)]. Alongm this line, ACKR3 is highly upregulated |
| | | | during monocyte-to-macrophage differentiation in vitro, |
| | | | switching to a more pro-inflammatory cell phenotype (Ma et al., |
| | | | 2013; Chatterjee et al., 2015). Another example can be found |
| | | | during central nervous system inflammation, where ACKR3 is |
| | | | upregulated in endothelial cells of the blood-brain barrier(Cruz- |
| | | | Orengo et al., 2011). Antagonizing the scavenging activity of |
| | Down | ENSMUSG00000018819 | Lymphocyte-specific protein 1 may regulate neutrophil mobility, |
| | | | adhesion to fibrinogen matrix protein, and transendothelial |
| | | | migration. LSP (lymphocyte-specific protein) 1 as a critical |
| | | | regulator of actomyosin contractility in primary macrophages. |
| | | | LSP1 regulates adhesion and migration, including the |
| | Down | ENSMUSG00000038400 | Prostate transmembrane protein, andeogen induced 1 |
| | | | (PMEPA1), is induced by the TGF?? signalling, but meanwhile, it |
| | | | inhibits the phosphorylation of Smad2 and Smad3 to antagonize |
| | | | TGF?? signalling. PMEPA1 activates the bone morphogenetic |
| | | | proteins (BMP) signalling of TGF?? signalling resulting in |
| | Down | ENSMUSG00000030707 | Coro1A belongs to a family of evolutionary conserved |
| | | | actinbinding proteins that regulate actin cytoskeleton- |
| | | | dependent processes such as cytokinesis, cell polarization, |
| | | | migration, and phagocytosis. In the mammalian system, Coro1A |
| | | | is predominantly expressed in |
| | | | leukocytes and plays an important role, for example, in Ca2+ |
| | | | signaling in macrophages, TCR signaling, and lymphocyte |
| | Down | ENSMUSG00000018339 | Gltathione peroxidase-3 is an enzyme that functions in |
| | | | detoxification of hydrogen peroxide. GPX3 plays a pivotal role |
| | | | in arterial and venous thrombosis. GPX3 maintains the |
| | | | bioavailability of nitric oxide (NO) in the vascular system, and |
| | | | GPX3 deficiency leads to the decreased vascular bioavailability |
| | | | of NO, which attenuates its effect on platelet function and |
| | | | subsequently results in a prothrombotic state. Decreased GPX3 |
| Vascular | Down | ENSMUSG00000055775 | Myosin heavy chain 8 encodes a member of the class II or |
| regulation | | | conventional myosin heavy chain and functions in skeletal |
| | Down | ENSMUSG00000041616 | Natriuretic peptide A (Nppa) encoding arterial natruretic |
| | | | peptide (ANP) belongs to the natriuretic peptide family, is |
| | | | implicated in the decrease of blood preasure and inhibition of |
| | Down | ENSMUSG00000007877 | Telethinin, also known as Tcap, is expressed in cardiac and |
| | | | skeletal muscle at Z-disc and functions to regulate sarcomere |
| | Down | ENSMUSG00000068699 | Filamin C expression is restricted to striated muscles and |
| | | | localizes around the Z?disc, the sarcolemma, the myotendinous |
| | | | junction, and the intercalated discs. Its main role is maintaining |
| | | | the structural integrity of the sarcomere. This is through |
| | | | crosslinking actin filaments and the anchoring of sarcolemmal |
| | Down | ENSMUSG00000020264 | Solute carrier family 36 member 2 is a pH-dependent proton- |
| | | | coupled amino acid transporter that belongs to the amino acid |
| | | | auxin permease 1 protein family and primarily transports small |
| | | | amino acids such as glycine, alanine and proline.. SLC36A2 is |
| | | | expressed at the apical surface of the human renal proximal |
| | | | tubule where it functions in the reabsorption of glycine, proline |
| | | | and hydroxyproline. SLC36A2 also transports amino acid |
| | Down | ENSMUSG00000040752 | Myosin heavy chain 6 gene provides instruction for making a |
| | | | protein known as the cariac alpha-myosin heavy chain. |
| | Down | ENSMUSG00000068614 | Cardiac muscle alpha actin is the major protein of the thin |
| | | | filament in cardiac sarcomeres, which are responsible for |
| | | | muscle contraction and generation of force to support the pump |
| | Down | ENSMUSG000000035783 | smooth muscle actin or a-SMA, often used as a marker of |
| | | | myofibroblast formation. |
| | Down | ENSMUSG00000028464 | Tropomyosin beta chain is striated muscle-specific coiled coil |
| | | | dimer that functions to stabilize actin filaments and regulate |

| [symbol] | [symbol] | [symbol] | [symbol] | [symbol] |
|---|---|---|---|---|
| [symbol] | [symbol] | [symbol] | [symbol] | [symbol] |
| | [symbol] | [symbol] | [symbol] | [symbol] |
| | [symbol] | [symbol] | [symbol] | [symbol] |
| | [symbol] | [symbol] | [symbol] | [symbol] |
| | [symbol] | [symbol] | [symbol] | [symbol] |
| | [symbol] | [symbol] | [symbol] | [symbol] |
| | [symbol] | [symbol] | [symbol] | [symbol] |
| Senenscent cell | Up | ENSMUSG00000029188 | Li, Y., et al., Knockdown of | Promote hepatocyte |
| regulation | | | SLC34A2 Inhibits Hepatocellular | proliferation and |
| | | | Carcinoma Cell Proliferation | migration |
| | | | and Invasion. Oncology | |
| | | | Research, Vol. 24, pp. | |
| | | | 511-519, 2016. | |
| | Down | ENSMUSG00000007655 | Pol A. et al., Non-caveolar | reduce senescent cells |
| | | | caveolins duites outside the | |
| | | | caves J Cell Sci 2020; 133, | |
| | | | jcs241562; 2. Volonte D. et | |
| | | | al., Caveolin 1, a master | |
| | | | regulator of cellular | |
| | | | senescence; 3. Samarakoon, | |
| | | | R., et al., The TGF-?1/p53/ | |
| | | | PAI-1 Signaling Axis in | |
| | | | Vascular Senescence: Role of | |
| | | | Caveolin 1. | |
| | | | Biomolecules 2019, 9, 341; | |
| | Down | ENSMUSG00000037419 | Qiu, J., et al., Identification | reduce G0/G1 cell |
| | | | of endonuclease domaincontaining | cycle arrest |
| | | | 1 as a novel tumor suppressor | |
| | | | in prostate cancer. BMC Cancer | |
| | | | (2017) 17: 360 | |
| Inflammation | Up | ENSMUSG00000032578 | Palmer, DC., et al., Cish | inhibit T cell |
| regulation | | | actively silences TCR signaling | activation |
| | | | in CD8+ T cells to maintain | |
| | Down | ENSMUSG00000030017 | 1. Xu, X., et al., The Link | May reflect a reduction |
| | | | between Type III Reg and STAT3- | of inflammation |
| | | | Associated Cytokines in | |
| | | | Inflamed Colonic Tissues. | |
| | | | Mediators of Inflammation 2019; | |
| | | | 2019: 7859460. 2. Detection | |
| | | | of Reg3? by Immunohisto- | |
| | | | chemistry in Cerulein-Induced | |
| | | | Model of Acute Pancreatic Injury | |
| | | | in Mice and Rats Pancreas 48: 8, | |
| | Down | ENSMUSG00000076615 | | downregulation of B |
| | Down | ENSMUSG00000076613 | | downregulation of B |
| | Down | ENSMUSG00000064057 | 1. Xu, N., et al., Spatiotemporal | reduce inflammation |
| | | | Expression of Three Secretoglobin | |
| | | | Proteins, SCGB1A1, SCGB3A1, and | |
| | | | SCGB3A2, in Mouse Airway | |
| | | | Epithelia. Journal of | |
| | | | Histochemistry & Cytochemistry | |
| | | | 2019, Vol. 67(6) 453-463; 2. | |
| | | | Yamada A. and Kimura, S. | |
| | | | Induction of uteroglobin- | |
| | | | related protein 2 (Ugrp2) | |
| | | | expression by EGF and TGF?. | |
| | | | FEBS Lett. | |
| | Down | ENSMUSG00000022491 | Williams, P A., et al., | Increase lymphocyte |
| | | | GlyCAM1 negatively regulates | extravasation. |
| | | | monocyte entry into the optic | |
| | | | nerve head and contributes to | |
| | | | radiation-based protection in | |
| | | | glaucoma. Journal of | |
| | | | Neuroinflammation (2017) 14: 93 | |
| | Down | ENSMUSG00000076612 | | may relate to |
| | | | | downregulation of B |
| | Down | ENSMUSG00000076609 | | may relate to |
| | | | | downregulation of B |
| | Down | ENSMUSG00000032496 | Liang L., et al., Distribution | reduce neutrophil |
| | | | of Lactoferrin Is Related with | infiltration |
| | | | Dynamics of Neutrophils in | |
| | | | Bacterial Infected Mice | |
| | | | Intestine. Molecules 2020, 25, | |
| | | | 1496 | |
| | Down | ENSMUSG00000024673 | Cell Immunol 360, 2021, 104260 | may relate to |
| | | | | downregulation of B |
| | Down | ENSMUSG00000067149 | | may relate to |
| | | | | downregulation of B |
| | Down | ENSMUSG00000030724 | Morbach, H., et al., CD19 | may relate to |
| | | | controls TLR9 responses in | downregulation of B |
| | | | human B cells. J Allergy Clin | cell function |
| | | | Immunol. 2016 March; 137(3): | |
| | | | 889-898. | |
| | Down | ENSMUSG00000076617 | | may relate to |
| | | | | downregulation of B |
| | Down | ENSMUSG00000079017 | PLoS One 2014; 9(9): e106392 | may relate to |
| | | | | downregulation of |
| | Down | ENSMUSG00000044337 | Mol Pharmacol 96: 809-818, | ACKR3 is down that |
| | | | December 2019 | may be a sign of |
| | | | | inflammation is down |
| | Down | ENSMUSG00000018819 | NATURE COMMUNICATIONS \| | reduce inflammation |
| | | | (2018) 9: 515 | |
| | Down | ENSMUSG00000038400 | Zhang, L., et al., PMEPA1 | reflect a lower TGFB1 |
| | | | induces EMT via a non?canonical | levels |
| | | | TGF?? signalling in | |
| | | | colorectal cancer. J Cell Mol | |
| | | | Med. 2019; 23: 3603-3615. | |
| | Down | ENSMUSG00000030707 | Pick, R., et al., Coronin 1A, | reduce adoptive and |
| | | | a novel player in integrin | innate immunity |
| | | | biology, controls neutrophil | |
| | | | trafficking in innate immunity. | |
| | | | BLOOD, 17 Aug. 2017 | |
| | | | VOLUME 130, NUMBER 7 | |
| | Down | ENSMUSG00000018339 | Chen-Yu Chien, CY., et al., | increase ROS levels |
| | | | Glutathione peroxidase 3 gene | |
| | | | polymorphisms and the risk of | |
| | | | sudden sensorineural hearing | |
| | | | loss. Kaohsiung Journal of | |
| | | | Medical Sciences (2017) 33, | |
| | | | 359e364 | |
| Vascular | Down | ENSMUSG00000055775 | https://www.ncbi.nlm.nih.gov/ | muscle related |
| regulation | | | gtr/genes/4626/ | |
| | Down | ENSMUSG00000041616 | Handb Exp Pharmacol. 2009; | Increase blood |
| | | | (191): 341-366. | preasure and cardiac |
| | | | | hypertrophy/fibrosis |
| | Down | ENSMUSG00000007877 | https://en.wikipedia.org/wiki/ | muscle related |
| | | | Telethonin | |
| | Down | ENSMUSG00000068699 | Verdonschot, J A J., et al., A | muscle related |
| | | | mutation update for the FLNC | |
| | | | gene in myopathies and | |
| | | | cardiomyopathies. Human | |
| | | | Mutation. 2020; 41: 1091-1111. | |
| | Down | ENSMUSG00000020264 | Thwaites, D T., and Anderson, | reduce amino acid |
| | | | CMH., The SLC36 family of | derivatives |
| | | | proton-coupled amino acid | transportation |
| | | | transporters and their | |
| | | | potential role in drug | |
| | | | transport. British Journal of | |
| | | | Pharmacology (2011) 164 | |
| | | | 1802-1816 | |
| | Down | ENSMUSG00000040752 | https://medlineplus.gov/ | muscle related |
| | | | genetics/gene/myh6/ | |
| | Down | ENSMUSG00000068614 | https://en.wikipedia.org/ | Muscle related |
| | | | wiki/ACTC1 | |
| | Down | ENSMUSG00000035783 | https://en.wikipedia.org/wiki/ | muscle related |
| | | | ACTA2 | |
| | Down | ENSMUSG00000028464 | https://en.wikipedia.org/wiki/ | muscle related |
| | | | TPM2 | |

[symbol]

indicates data missing or illegible when filed

### Example 70: RNA-Seq Analysis of Differentially Expressed Genes Between the PBS (Control Group) or TGFRt15-TGFRs (TGFRt15-TGFRs Group) in Aged Mice Liver

C57BL/6, 72-week-old mice were purchased from the Jackson Laboratory. Mice were housed in a temperature and light controlled environment. Mice were divided into two groups and treated subcutaneously with either PBS (PBS control group) or TGFRt15-TGFRs at a dosage of 3 mg/kg (TGFRt15-TGFRs group). At day 60 post treatment, mice were euthanized, and livers were harvested. Harvested livers were stored in liquid nitrogen in 1.7 mL Eppendorf tubes. Samples were homogenized by using homogenizer in 1 mL of Trizol (Thermo Fischer). Homogenized tissues were transferred in fresh Eppendorf tubes. Total RNA was extracted using RNeasy Mini Kit (Qiagen #74106) according to the manufacturer's instructions.

Library preparations, sequencing reactions and bioinformatic analysis were conducted at GENEWIZ, LLC. (South Plainfield, NJ, USA) as follows: Library preparation with poly A selection and HiSeq sequencing extracted RNA samples were quantified using Qubit 2.0 Fluorometer (Life Technologies, Carlsbad, CA, USA) and RNA integrity was checked using Agilent TapeStation 4200 (Agilent Technologies, Palo Alto, CA, USA). RNA sequencing libraries were prepared using the NEBNext Ultra II RNA Library Prep Kit for Illumina following manufacturer's instructions (NEB, Ipswich, MA, USA). Briefly, mRNAs were first enriched with oligo (dT) beads. Enriched mRNAs were fragmented for 15 minutes at 94° C. First strand and second strand cDNAs were subsequently synthesized and cDNA fragments were end repaired and adenylated at 3′ends. Universal adapters were ligated to cDNA fragments, followed by index addition and library enrichment by limited-cycle PCR. The sequencing libraries were validated on the Agilent TapeStation (Agilent Technologies, Palo Alto, CA, USA), and quantified by using Qubit 2.0 Fluorometer (Invitrogen, Carlsbad, CA) as well as by quantitative PCR (KAPA Biosystems, Wilmington, MA, USA). The sequencing libraries were clustered on 1 flowcell lane. After clustering, the flowcell was loaded on the Illumina HiSeq instrument (4000 or equivalent) according to manufacturer's instructions. The samples were sequenced using a 2×150 bp Paired End (PE) configuration. Image analysis and base calling were conducted by the HiSeq Control Software (HCS). Raw sequence data (.bcl files) generated from Illumina HiSeq was converted into fastq files and de-multiplexed using Illumina's bcl2fastq 2.17 software. One mismatch was allowed for index sequence identification. Sequence reads were trimmed to remove possible adapter sequences and nucleotides with poor quality using Trimmomatic v.0.36. The trimmed reads were mapped to the Mus musculus GRCm38 reference genome available on ENSEMBL using the STAR aligner v.2.5.2b. The STAR aligner is a splice aligner that detects splice junctions and incorporates them to help align the entire read sequences. BAM files were generated as a result of this step. Unique gene hit counts were calculated by using feature counts from the Subread package v.1.5.2. The hit counts were summarized and reported using the gene_id feature in the annotation file. Only unique reads that fell within exon regions were counted. If a strand-specific library preparation was performed, the reads were strand-specifically counted. After extraction of gene hit counts, the gene hit counts table was used for downstream differential expression analysis. Using DESeq2, a comparison of gene expression between the treatment-specific groups of samples was performed. The Wald test was used to generate p-values and log 2 fold changes. Genes with an adjusted p-value<0.05 and absolute log 2 fold change>1 were called as differentially expressed genes for each comparison. A gene ontology analysis was performed on the statistically significant set of genes by implementing the software GeneSCF v.1.1-p2. The mgi GO list was used to cluster the set of genes based on their biological processes and determine their statistical significance. To estimate the expression levels of alternatively spliced transcripts, the splice variant hit counts were extracted from the RNA-seq reads mapped to the genome. Differentially spliced genes were identified for groups with more than one sample by testing for significant differences in read counts on exons (and junctions) of the genes using DEXSeq. For groups with only one sample, the exon hit count tables were provided.

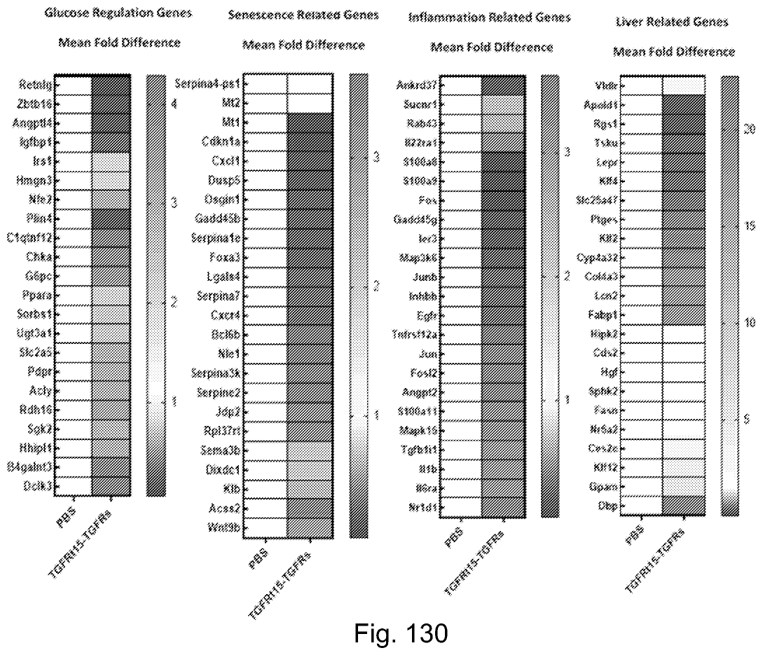

The significant genes downregulated or upregulated were divided into four groups according to the function. The mean fold change was calculated by dividing the experimental group by the mean the control group. The heatmaps were constructed with GraphPad in accordance with gene functions. As showed in FIG. 130 and Tables 3 and 4, most senescence and inflammation genes were downregulated in livers of the TGFRt15-TGFRs treated group compared to the PBS control group.

TABLE 3
RNA-seq analysis of differentially expressed genes between the PBS (Control
Group) or TGFRt15-TGFRs (TGFRt15-TGFRs group) in aged mice liver

| ID | log2FoldChange | pvalue | padj | agedliver92181tox | agedliver92182tox | agedliver92183tox |
|---|---|---|---|---|---|---|
| ENSMUSG00000000204 | −2.005879896 | 3.88E−05 | 0.000867087 | 8.974563618 | 11.65468178 | 11.93770314 |
| ENSMUSG00000000317 | −1.462090887 | 1.76E−06 | 7.11E−05 | 60.82759785 | 43.70505667 | 55.0970914 |
| ENSMUSG00000000686 | 1.151464201 | 2.84E−06 | 0.000104278 | 355.9910235 | 571.0794072 | 469.2435618 |
| ENSMUSG00000001227 | −1.099312542 | 1.49E−06 | 6.21E−05 | 100.7145473 | 67.98564371 | 92.74677053 |
| ENSMUSG00000001403 | −1.612087212 | 0.002110809 | 0.017174878 | 16.9519535 | 6.798564371 | 11.01941828 |
| ENSMUSG00000001983 | 1.013926416 | 1.20E−05 | 0.000332241 | 308.1266842 | 343.8131125 | 299.3608633 |
| ENSMUSG00000002233 | −1.270303057 | 1.05E−10 | 1.46E−08 | 133.6212805 | 104.892136 | 141.4158679 |
| ENSMUSG00000002250 | −1.142106413 | 9.77E−06 | 0.000282853 | 303.1408155 | 156.3669805 | 415.9830401 |
| ENSMUSG00000002289 | −2.433615292 | 5.35E−35 | 1.82E−31 | 340.0362437 | 320.5037489 | 528.0137926 |
| ENSMUSG00000002831 | −2.428567233 | 6.40E−14 | 1.90E−11 | 67.807814 | 71.87053764 | 51.42395197 |
| ENSMUSG00000003032 | −1.814709673 | 6.30E−10 | 7.35E−08 | 33.903907 | 28.16548097 | 44.99595798 |
| ENSMUSG00000003348 | 1.035062446 | 0.006673687 | 0.038727262 | 47.86433929 | 51.47484453 | 59.68851569 |
| ENSMUSG00000003500 | −1.169914769 | 9.39E−05 | 0.001721093 | 55.84172918 | 53.41729149 | 52.34223683 |
| ENSMUSG00000003541 | −2.579058324 | 2.05E−08 | 1.65E−06 | 9.971737353 | 11.65468178 | 11.01941828 |
| ENSMUSG00000003848 | −1.114660438 | 1.53E−05 | 0.000400378 | 168.5223613 | 191.3310259 | 135.9061588 |
| ENSMUSG00000004100 | −2.152000208 | 2.40E−05 | 0.000578522 | 177.4969249 | 181.6187911 | 165.2912742 |
| ENSMUSG00000004933 | −1.661199536 | 0.00641801 | 0.037574902 | 11.96608482 | 8.741011335 | 3.673139427 |
| ENSMUSG00000004951 | −1.245761383 | 7.16E−05 | 0.001407433 | 177.4969249 | 194.2446963 | 114.7856071 |
| ENSMUSG00000005148 | −1.641216558 | 0.00407345 | 0.027163637 | 8.974563618 | 3.884893927 | 9.182848567 |
| ENSMUSG00000005547 | 1.316837452 | 9.30E−15 | 3.02E−12 | 9129.125547 | 13948.71164 | 12101.15784 |
| ENSMUSG00000005580 | 1.4493211 | 2.75E−08 | 2.14E−06 | 248.2962601 | 371.00737 | 337.0105424 |
| ENSMUSG00000006050 | −1.042916307 | 2.32E−07 | 1.27E−05 | 992.1878666 | 1015.899762 | 869.6157593 |
| ENSMUSG00000006134 | 1.201348738 | 1.44E−09 | 1.52E−07 | 677.0809663 | 610.8995699 | 678.6125091 |
| ENSMUSG00000006517 | 1.182376585 | 1.39E−07 | 8.18E−06 | 2010.30225 | 1472.374798 | 1714.437827 |
| ENSMUSG00000006587 | 1.922052303 | 0.003206819 | 0.022932036 | 30.91238579 | 14.56835222 | 21.1205517 |
| ENSMUSG00000006711 | 1.246016282 | 4.07E−11 | 5.91E−09 | 435.7649223 | 410.8275327 | 410.4733309 |
| ENSMUSG00000006777 | 1.982368887 | 0.000106088 | 0.001901083 | 22.93499591 | 53.41729149 | 33.05825484 |
| ENSMUSG00000008153 | 1.444860417 | 1.72E−08 | 1.42E−06 | 278.2114721 | 339.9282186 | 185.4935411 |
| ENSMUSG00000009013 | −1.025154388 | 6.49E−07 | 3.04E−05 | 138.6071492 | 163.1655449 | 192.8398199 |
| ENSMUSG00000009633 | −1.150697898 | 7.41E−07 | 3.38E−05 | 1177.662181 | 1721.008009 | 1060.619009 |
| ENSMUSG00000014547 | 1.47853344 | 5.88E−06 | 0.00018548 | 76.78237762 | 107.8058065 | 108.3576131 |
| ENSMUSG00000014609 | 1.198939285 | 0.004891473 | 0.030982358 | 34.90108074 | 33.99282186 | 34.89482455 |
| ENSMUSG00000015224 | −1.47719251 | 7.85E−07 | 3.55E−05 | 57.83607665 | 125.2878291 | 79.89078253 |
| ENSMUSG00000015312 | −2.170653759 | 1.83E−07 | 1.04E−05 | 27.92086459 | 16.51079919 | 10.10113342 |
| ENSMUSG00000016128 | 1.194768611 | 1.81E−09 | 1.86E−07 | 486.6207828 | 535.1441384 | 631.7799814 |
| ENSMUSG00000016356 | −1.303561468 | 6.94E−06 | 0.00021464 | 50.8558605 | 59.24463238 | 46.83252769 |
| ENSMUSG00000017737 | −1.495519922 | 0.005185903 | 0.032307064 | 19.94347471 | 7.769787853 | 5.50970914 |
| ENSMUSG00000017868 | 1.520950393 | 2.42E−16 | 1.03E−13 | 1574.537328 | 1919.1376 | 1179.077756 |
| ENSMUSG00000018486 | 1.561119026 | 0.006908525 | 0.039768921 | 26.92369085 | 21.3669166 | 27.5485457 |
| ENSMUSG00000019082 | −1.214544808 | 1.93E−12 | 4.05E−10 | 2583.677148 | 3303.131061 | 2721.796315 |
| ENSMUSG00000019726 | 1.436923585 | 3.39E−10 | 4.31E−08 | 430.7790536 | 552.626161 | 627.1885571 |
| ENSMUSG00000019737 | −1.672592181 | 0.00414668 | 0.027410702 | 8.974563618 | 9.712234816 | 6.427993997 |
| ENSMUSG00000019883 | 1.383035463 | 1.73E−09 | 1.79E−07 | 790.7587721 | 685.683778 | 707.0793397 |
| ENSMUSG00000020018 | −1.224865075 | 5.55E−07 | 2.68E−05 | 76.78237762 | 100.0360186 | 117.5404617 |
| ENSMUSG00000020027 | −2.148540831 | 0.000201757 | 0.00309142 | 308.1266842 | 206.8706016 | 291.0962996 |
| ENSMUSG00000020091 | 1.323362559 | 0.005934886 | 0.035409378 | 1482.797344 | 1694.784975 | 1814.530877 |
| ENSMUSG00000020122 | −1.730853584 | 6.04E−21 | 5.49E−18 | 534.4851221 | 692.4823424 | 689.6319274 |
| ENSMUSG00000020335 | 1.099463556 | 0.002629472 | 0.020167667 | 41.88129688 | 88.38133683 | 80.80906739 |
| ENSMUSG00000020429 | −1.914789163 | 0.005628577 | 0.034083881 | 1082.930677 | 219.4965068 | 977.0550875 |
| ENSMUSG00000020441 | −1.319584205 | 1.17E−07 | 7.17E−06 | 96.72585232 | 114.6043708 | 87.23706139 |
| ENSMUSG00000020532 | 1.125678465 | 6.99E−06 | 0.000215157 | 6805.710743 | 2936.008585 | 5607.047335 |
| ENSMUSG00000020641 | −1.035196027 | 2.26E−05 | 0.00055792 | 307.1295105 | 181.6187911 | 139.5792982 |
| ENSMUSG00000020656 | −2.17958622 | 1.43E−08 | 1.21E−06 | 16.9519535 | 7.769787853 | 23.87540627 |
| ENSMUSG00000020681 | 1.270736177 | 0.003452779 | 0.024158824 | 30.91238579 | 35.93526882 | 52.34223683 |
| ENSMUSG00000020692 | −1.373008874 | 7.68E−06 | 0.000232298 | 53.84738171 | 65.07197327 | 76.21764311 |
| ENSMUSG00000020812 | −1.318469089 | 0.000692163 | 0.007551219 | 32.90673326 | 43.70505667 | 47.75081255 |
| ENSMUSG00000020889 | 1.857219295 | 5.65E−19 | 3.50E−16 | 1005.151125 | 1714.209445 | 1803.511459 |
| ENSMUSG00000020917 | 1.579449336 | 7.98E−12 | 1.40E−09 | 34685.69121 | 17873.42573 | 22782.64729 |
| ENSMUSG00000020948 | 1.248898073 | 0.003935164 | 0.026500655 | 67.807814 | 53.41729149 | 56.93366111 |
| ENSMUSG00000020961 | 1.570215029 | 7.42E−07 | 3.38E−05 | 116.669327 | 116.5468178 | 141.4158679 |
| ENSMUSG00000021250 | −3.196150425 | 1.40E−18 | 7.95E−16 | 14.95760603 | 8.741011335 | 25.71197599 |
| ENSMUSG00000021260 | 1.563177947 | 0.002093122 | 0.017081929 | 31.90955953 | 29.13670445 | 23.87540627 |
| ENSMUSG00000021416 | 1.171425747 | 1.31E−08 | 1.13E−06 | 910.4196203 | 675.9715432 | 629.0251268 |
| ENSMUSG00000021453 | −2.706070458 | 5.59E−23 | 6.28E−20 | 149.5760603 | 61.18707934 | 130.3964497 |
| ENSMUSG00000021611 | 1.027773708 | 1.21E−09 | 1.30E−07 | 416.8186214 | 423.453438 | 475.6715558 |
| ENSMUSG00000021670 | 1.206582161 | 2.43E−07 | 1.30E−05 | 4843.272832 | 4623.994996 | 5239.733392 |
| ENSMUSG00000021684 | −1.138260866 | 0.001182935 | 0.011115132 | 32.90673326 | 25.25181052 | 22.03883656 |
| ENSMUSG00000021773 | −1.294178405 | 0.002327317 | 0.01846284 | 17.94912724 | 12.62590526 | 22.03883656 |
| ENSMUSG00000021775 | 1.550962326 | 2.41E−10 | 3.16E−08 | 1168.687618 | 1405.360378 | 1119.38924 |
| ENSMUSG00000021804 | 1.339955479 | 0.00532128 | 0.032924815 | 26.92369085 | 35.93526882 | 37.64967912 |
| ENSMUSG00000021958 | −1.041099878 | 0.007096785 | 0.040595156 | 25.92651712 | 21.3669166 | 47.75081255 |
| ENSMUSG00000022383 | 1.183413475 | 3.19E−08 | 2.41E−06 | 1160.710228 | 1260.648079 | 1669.441869 |
| ENSMUSG00000022389 | 1.619123522 | 2.95E−16 | 1.22E−13 | 1357.153454 | 2316.368004 | 1561.084256 |
| ENSMUSG00000022408 | 1.528188008 | 2.54E−06 | 9.66E−05 | 74.78803015 | 107.8058065 | 82.6456371 |
| ENSMUSG00000022528 | −1.557702695 | 1.66E−19 | 1.13E−16 | 205.4177895 | 185.503685 | 197.4312442 |
| ENSMUSG00000022651 | −4.171544663 | 2.34E−09 | 2.36E−07 | 3.988694941 | 2.913670445 | 0 |
| ENSMUSG00000022704 | −1.016808687 | 0.000177103 | 0.002808323 | 94.73150485 | 108.7770299 | 100.0930494 |
| ENSMUSG00000022853 | 1.04479536 | 2.29E−11 | 3.59E−09 | 9525.00352 | 10073.52995 | 7460.146176 |
| ENSMUSG00000022883 | 1.330834067 | 1.00E−09 | 1.09E−07 | 280.2058196 | 336.0433246 | 355.3762395 |
| ENSMUSG00000022887 | 1.363326654 | 1.68E−07 | 9.74E−06 | 1196.608482 | 1241.22361 | 1538.127135 |
| ENSMUSG00000022911 | 1.024722285 | 3.96E−05 | 0.000880701 | 133.6212805 | 188.4173554 | 193.7581048 |
| ENSMUSG00000023034 | −2.875895339 | 0.000200077 | 0.003084527 | 58.83325038 | 34.96404534 | 22.95712142 |
| ENSMUSG00000023044 | −1.350199698 | 7.38E−12 | 1.31E−09 | 1875.683796 | 2461.080302 | 2315.914409 |
| ENSMUSG00000023052 | −1.334599319 | 0.004914148 | 0.031082668 | 13.96043229 | 11.65468178 | 17.44741228 |
| ENSMUSG00000023067 | −3.022867976 | 2.66E−57 | 3.63E−53 | 136.6128017 | 95.1799012 | 134.9878739 |
| ENSMUSG00000023073 | 1.225822592 | 2.02E−05 | 0.00050363 | 443.7423122 | 525.4319036 | 592.2937326 |
| ENSMUSG00000023341 | −1.14606456 | 1.95E−06 | 7.71E−05 | 89.74563618 | 99.06479513 | 85.40049167 |
| ENSMUSG00000023571 | −1.317554169 | 0.002212852 | 0.017761424 | 18.94630097 | 21.3669166 | 14.69255771 |
| ENSMUSG00000023800 | 1.312092825 | 2.45E−12 | 4.99E−10 | 459.697092 | 410.8275327 | 456.3875738 |
| ENSMUSG00000023905 | −1.475845141 | 9.44E−06 | 0.000275028 | 54.84455544 | 95.1799012 | 81.72735225 |
| ENSMUSG00000023927 | −1.218211099 | 0.003077602 | 0.022366209 | 27.92086459 | 26.223034 | 12.85598799 |
| ENSMUSG00000023968 | −1.267291327 | 0.008148531 | 0.04468095 | 10.96891109 | 11.65468178 | 15.61084256 |
| ENSMUSG00000024118 | −1.757053931 | 5.31E−18 | 2.79E−15 | 442.7451385 | 592.4463238 | 352.621385 |
| ENSMUSG00000024130 | 1.042830636 | 1.30E−05 | 0.000354612 | 2989.526858 | 2745.648783 | 3530.805274 |
| ENSMUSG00000024136 | −1.225506878 | 0.002002269 | 0.016518413 | 27.92086459 | 19.42446963 | 17.44741228 |
| ENSMUSG00000024190 | −1.579039564 | 2.17E−13 | 5.48E−11 | 281.2029934 | 502.12254 | 460.0607132 |
| ENSMUSG00000024236 | 1.389584652 | 9.49E−13 | 2.12E−10 | 1344.190195 | 1041.151572 | 1299.373072 |
| ENSMUSG00000024411 | 1.68012077 | 5.07E−05 | 0.001068968 | 63.81911906 | 48.56117408 | 35.81310941 |
| ENSMUSG00000024440 | 1.007637941 | 0.000827422 | 0.008613399 | 107.6947634 | 103.9209125 | 147.8438619 |
| ENSMUSG00000024665 | 1.18951247 | 3.11E−12 | 6.05E−10 | 9244.7977 | 8635.147975 | 9906.457034 |
| ENSMUSG00000024843 | −1.175648724 | 2.45E−11 | 3.79E−09 | 1421.969747 | 1016.870985 | 1263.559963 |
| ENSMUSG00000024887 | 1.067323547 | 1.55E−06 | 6.39E−05 | 398.8694941 | 436.0793433 | 565.6634717 |
| ENSMUSG00000024924 | 1.265415535 | 2.60E−12 | 5.20E−10 | 642.1798855 | 816.7989481 | 589.538878 |
| ENSMUSG00000024970 | −1.174479585 | 0.000322102 | 0.004379373 | 31.90955953 | 39.82016275 | 25.71197599 |
| ENSMUSG00000024978 | 2.527860907 | 4.51E−08 | 3.23E−06 | 10405.50793 | 5475.757989 | 8311.396238 |
| ENSMUSG00000025003 | 1.045766059 | 5.74E−07 | 2.75E−05 | 301.1464681 | 286.5109271 | 410.4733309 |
| ENSMUSG00000025006 | 1.229102652 | 2.44E−06 | 9.41E−05 | 288.1832095 | 294.2807149 | 416.9013249 |
| ENSMUSG00000025153 | 1.113433094 | 9.44E−07 | 4.18E−05 | 85604.37365 | 41744.15646 | 57005.28733 |
| ENSMUSG00000025161 | −1.470478397 | 0.001962044 | 0.016285081 | 18.94630097 | 9.712234816 | 14.69255771 |
| ENSMUSG00000025240 | 1.333245056 | 1.72E−06 | 6.99E−05 | 553.4314231 | 682.7701076 | 696.0599214 |
| ENSMUSG00000025323 | 1.252220218 | 7.14E−07 | 3.28E−05 | 138.6071492 | 127.2302761 | 172.6375531 |
| ENSMUSG00000025402 | −1.340941683 | 6.59E−15 | 2.25E−12 | 217.3838743 | 245.7195409 | 214.8786565 |
| ENSMUSG00000025429 | 1.693186246 | 2.17E−13 | 5.48E−11 | 624.2307583 | 540.0002558 | 651.9822483 |
| ENSMUSG00000025450 | −1.257708301 | 2.83E−05 | 0.000669372 | 30.91238579 | 62.15830282 | 55.0970914 |
| ENSMUSG00000025997 | −1.284615532 | 0.009041141 | 0.048049118 | 26.92369085 | 9.712234816 | 12.85598799 |
| ENSMUSG00000026020 | −1.156643623 | 9.59E−08 | 6.11E−06 | 250.2906076 | 294.2807149 | 269.9757479 |
| ENSMUSG00000026249 | −1.326755053 | 0.000451208 | 0.005606296 | 36.89542821 | 50.50362104 | 26.63026084 |
| ENSMUSG00000026358 | −2.793399328 | 5.66E−13 | 1.33E−10 | 12.96325856 | 11.65468178 | 12.85598799 |
| ENSMUSG00000026398 | 1.137826301 | 6.48E−12 | 1.16E−09 | 1403.023446 | 1667.590718 | 1539.04542 |
| ENSMUSG00000026471 | 1.000583106 | 1.46E−05 | 0.000384933 | 269.2369085 | 237.949753 | 315.8899907 |
| ENSMUSG00000026475 | −1.56210025 | 0.004964709 | 0.031300593 | 735.9142167 | 388.4893927 | 994.5024998 |
| ENSMUSG00000026525 | −1.790734538 | 0.001947167 | 0.0161813 | 7.977389882 | 8.741011335 | 6.427993997 |
| ENSMUSG00000026822 | −1.02651459 | 5.14E−06 | 0.00016724 | 122.6523694 | 119.4604882 | 110.1941828 |
| ENSMUSG00000026826 | −1.483020728 | 0.008821676 | 0.047306802 | 15.95477976 | 10.6834583 | 11.93770314 |
| ENSMUSG00000026832 | −1.174113684 | 0.001522033 | 0.013425591 | 30.91238579 | 34.96404534 | 25.71197599 |
| ENSMUSG00000027360 | −1.979964656 | 1.70E−12 | 3.69E−10 | 54.84455544 | 93.23745424 | 72.54450368 |
| ENSMUSG00000027398 | −1.057691022 | 0.001937845 | 0.016123489 | 78.77672509 | 37.87771578 | 32.13996998 |
| ENSMUSG00000027405 | −1.241634744 | 7.40E−06 | 0.000226395 | 225.3612642 | 254.4605522 | 248.8551962 |
| ENSMUSG00000027496 | −1.007831872 | 0.004782077 | 0.030501955 | 38.88977568 | 37.87771578 | 17.44741228 |
| ENSMUSG00000027513 | −1.115800194 | 1.04E−10 | 1.46E−08 | 16805.36896 | 18645.5484 | 18901.97549 |
| ENSMUSG00000027605 | 1.86697993 | 9.67E−16 | 3.77E−13 | 27327.54622 | 18714.50527 | 21561.32844 |
| ENSMUSG00000027762 | 1.17286147 | 2.98E−15 | 1.13E−12 | 1319.260852 | 1520.935972 | 1404.975831 |
| ENSMUSG00000027907 | −1.140239221 | 0.000303295 | 0.004203282 | 47.86433929 | 56.33096193 | 106.5210434 |
| ENSMUSG00000027947 | −1.05537492 | 2.15E−06 | 8.45E−05 | 434.7677486 | 468.1297181 | 461.8972829 |
| ENSMUSG00000028008 | 1.245889 | 0.000553467 | 0.00652119 | 54.84455544 | 81.58277246 | 55.0970914 |
| ENSMUSG00000028339 | 1.011959422 | 2.14E−08 | 1.71E−06 | 706.9961783 | 505.0362104 | 521.5857986 |
| ENSMUSG00000028445 | 1.767301879 | 0.003925136 | 0.026446185 | 70.79933521 | 71.87053764 | 91.82848567 |
| ENSMUSG00000028630 | 1.331166045 | 1.46E−07 | 8.59E−06 | 373.9401507 | 358.3814647 | 406.8001915 |
| ENSMUSG00000028838 | 1.693729472 | 3.36E−09 | 3.22E−07 | 304.1379893 | 151.5108631 | 198.349529 |
| ENSMUSG00000028859 | −1.001034699 | 0.004539011 | 0.029294129 | 74.78803015 | 57.30218542 | 29.38511541 |
| ENSMUSG00000028862 | −2.278242573 | 1.62E−07 | 9.45E−06 | 8.974563618 | 9.712234816 | 17.44741228 |
| ENSMUSG00000028864 | 1.08474786 | 0.000792949 | 0.008337279 | 131.6269331 | 134.0288405 | 203.8592382 |
| ENSMUSG00000028957 | 2.966674755 | 1.69E−27 | 3.84E−24 | 314.1097266 | 609.9283465 | 341.6019667 |
| ENSMUSG00000028976 | 1.532387058 | 3.09E−07 | 1.58E−05 | 157.5534502 | 94.20867772 | 101.9296191 |
| ENSMUSG00000029086 | 1.631659452 | 1.01E−18 | 6.00E−16 | 713.9763945 | 617.6981343 | 593.2120174 |
| ENSMUSG00000029135 | −1.266157753 | 3.55E−07 | 1.81E−05 | 179.4912724 | 127.2302761 | 136.8244436 |
| ENSMUSG00000029188 | −2.354954255 | 1.89E−12 | 4.02E−10 | 42.87847062 | 12.62590526 | 51.42395197 |
| ENSMUSG00000029195 | 1.272024305 | 6.31E−09 | 5.74E−07 | 705.0018309 | 838.1658646 | 788.8066919 |
| ENSMUSG00000029370 | 1.683633222 | 1.05E−19 | 7.55E−17 | 546.4512069 | 472.9858356 | 579.4377446 |
| ENSMUSG00000029373 | 1.650047987 | 0.002725238 | 0.020577678 | 7.977389882 | 5.82734089 | 9.182848567 |
| ENSMUSG00000029380 | −2.803403219 | 8.38E−26 | 1.43E−22 | 53.84738171 | 128.2014996 | 71.62621882 |
| ENSMUSG00000029580 | −1.162469269 | 9.11E−12 | 1.55E−09 | 8321.414821 | 7155.974613 | 6927.540959 |
| ENSMUSG00000029591 | −1.593385799 | 3.01E−08 | 2.29E−06 | 43.87564435 | 60.21585586 | 35.81310941 |
| ENSMUSG00000029656 | −1.019716092 | 0.000134204 | 0.002299171 | 189.4630097 | 409.8563092 | 189.1666805 |
| ENSMUSG00000030032 | −1.061169015 | 0.007631272 | 0.042668167 | 23.93216965 | 14.56835222 | 25.71197599 |
| ENSMUSG00000030055 | 1.305889925 | 4.54E−06 | 0.000151075 | 239.3216965 | 263.2015635 | 292.9328693 |
| ENSMUSG00000030691 | 1.035229834 | 1.01E−08 | 8.79E−07 | 365.9627609 | 443.8491311 | 415.0647552 |
| ENSMUSG00000030782 | −1.087204324 | 0.00053618 | 0.006369234 | 38.88977568 | 39.82016275 | 40.40453369 |
| ENSMUSG00000030814 | −1.357189589 | 8.78E−10 | 9.81E−08 | 261.2595186 | 283.5972566 | 291.0962996 |
| ENSMUSG00000030827 | −1.631143551 | 1.45E−05 | 0.000383464 | 26.92369085 | 20.39569311 | 33.05825484 |
| ENSMUSG00000030934 | 1.434845811 | 0.000702548 | 0.007652274 | 13671.25191 | 24780.76713 | 9134.17947 |
| ENSMUSG00000030968 | −1.140744433 | 0.000133867 | 0.002296287 | 34.90108074 | 72.84176112 | 54.17880654 |
| ENSMUSG00000031010 | 1.281779593 | 3.26E−07 | 1.66E−05 | 953.2980909 | 1398.561814 | 1420.586673 |
| ENSMUSG00000031271 | −1.719181951 | 2.91E−23 | 3.97E−20 | 339.03907 | 470.0721651 | 364.5590881 |
| ENSMUSG00000031378 | 1.154152634 | 6.32E−15 | 2.21E−12 | 1014.125689 | 912.9500727 | 885.2266019 |
| ENSMUSG00000031465 | −1.254345174 | 0.0029265 | 0.021654196 | 21.93782218 | 11.65468178 | 20.20226685 |
| ENSMUSG00000031762 | −4.701992063 | 1.08E−05 | 0.000305681 | 384.9090618 | 188.4173554 | 32.13996998 |
| ENSMUSG00000031765 | −4.008275951 | 6.98E−08 | 4.64E−06 | 1129.797842 | 691.5111189 | 337.0105424 |
| ENSMUSG00000032009 | 1.096135097 | 4.98E−08 | 3.50E−06 | 816.6852892 | 873.12991 | 1044.089882 |
| ENSMUSG00000032064 | 1.076164714 | 3.75E−10 | 4.69E−08 | 659.131839 | 690.5398954 | 661.1650968 |
| ENSMUSG00000032083 | −1.056710751 | 2.91E−09 | 2.86E−07 | 57416.2665 | 56826.28591 | 68671.17815 |
| ENSMUSG00000032091 | 2.476316256 | 2.69E−06 | 0.000101175 | 102.7088947 | 50.50362104 | 32.13996998 |
| ENSMUSG00000032285 | 1.014640865 | 0.008899206 | 0.047498424 | 51.85303424 | 70.89931416 | 52.34223683 |
| ENSMUSG00000032417 | 1.412452049 | 0.000231229 | 0.003394264 | 102.7088947 | 50.50362104 | 80.80906739 |
| ENSMUSG00000032418 | 1.488424738 | 1.96E−09 | 2.00E−07 | 17569.20404 | 7712.485668 | 13799.06654 |
| ENSMUSG00000032500 | 2.09734091 | 1.65E−20 | 1.25E−17 | 413.8271001 | 367.1224761 | 372.8236518 |
| ENSMUSG00000032561 | 1.808480512 | 0.000942567 | 0.009458265 | 1361.142149 | 660.4319675 | 1176.322901 |
| ENSMUSG00000032702 | 1.072973429 | 8.45E−08 | 5.48E−06 | 1328.235415 | 1308.23803 | 1494.049462 |
| ENSMUSG00000032724 | 1.34458697 | 3.25E−10 | 4.18E−08 | 647.1657542 | 540.0002558 | 680.4490788 |
| ENSMUSG00000032735 | −1.26252931 | 1.21E−08 | 1.04E−06 | 191.4573572 | 257.3742226 | 294.769439 |
| ENSMUSG00000032786 | 1.26794906 | 3.89E−08 | 2.81E−06 | 1778.957944 | 2029.857077 | 2206.638511 |
| ENSMUSG00000032849 | 1.170479352 | 7.00E−10 | 8.09E−08 | 403.8553628 | 338.9569951 | 399.4539127 |
| ENSMUSG00000032860 | 1.046181292 | 6.20E−08 | 4.19E−06 | 339.03907 | 263.2015635 | 290.1780147 |
| ENSMUSG00000032883 | 1.139189345 | 1.88E−11 | 3.09E−09 | 1754.0286 | 1475.288469 | 1528.944286 |
| ENSMUSG00000033105 | 1.210153299 | 1.43E−10 | 1.97E−08 | 3593.814142 | 2741.763889 | 3240.627259 |
| ENSMUSG00000033594 | −1.045179866 | 5.30E−08 | 3.67E−06 | 379.9231931 | 342.841889 | 348.9482455 |
| ENSMUSG00000033624 | 1.545958738 | 3.43E−10 | 4.33E−08 | 290.177557 | 397.230404 | 428.8390281 |
| ENSMUSG00000033792 | 1.183185841 | 0.000561984 | 0.006589659 | 63.81911906 | 67.98564371 | 74.38107339 |
| ENSMUSG00000033855 | 1.291796081 | 0.000477464 | 0.005865923 | 117.6665008 | 69.92809068 | 198.349529 |
| ENSMUSG00000033967 | −2.822222129 | 4.36E−05 | 0.000952114 | 4.985868677 | 3.884893927 | 1.836569713 |
| ENSMUSG00000034066 | 1.2566902 | 2.38E−07 | 1.29E−05 | 275.2199509 | 346.7267829 | 310.3802816 |
| ENSMUSG00000034110 | 1.131917427 | 3.20E−05 | 0.000736514 | 99.71737353 | 110.7194769 | 115.7038919 |
| ENSMUSG00000034271 | −1.155793079 | 0.004733656 | 0.030334993 | 36.89542821 | 29.13670445 | 40.40453369 |
| ENSMUSG00000034755 | −1.51005032 | 0.006510973 | 0.037993212 | 7.977389882 | 5.82734089 | 12.85598799 |
| ENSMUSG00000034765 | −2.457798355 | 3.78E−06 | 0.000131464 | 23.93216965 | 12.62590526 | 9.182848567 |
| ENSMUSG00000034853 | 1.437062013 | 9.64E−14 | 2.63E−11 | 450.7225284 | 335.0721012 | 434.3487372 |
| ENSMUSG00000034926 | 1.093495822 | 1.31E−06 | 5.56E−05 | 17902.26007 | 19066.08817 | 17627.39611 |
| ENSMUSG00000035078 | 1.209517873 | 6.16E−05 | 0.001249206 | 322.0871165 | 384.6044987 | 346.193391 |
| ENSMUSG00000035112 | −1.146398835 | 0.004042609 | 0.02701081 | 23.93216965 | 14.56835222 | 18.36569713 |
| ENSMUSG00000035164 | 1.06213892 | 8.60E−06 | 0.000256206 | 131.6269331 | 166.0792154 | 141.4158679 |
| ENSMUSG00000035165 | −1.798528938 | 1.18E−06 | 5.05E−05 | 32.90673326 | 22.33814008 | 28.46683056 |
| ENSMUSG00000035284 | 1.432291641 | 4.22E−09 | 3.95E−07 | 725.9424793 | 940.1443302 | 1017.459621 |
| ENSMUSG00000035900 | 1.041020638 | 2.29E−08 | 1.81E−06 | 343.0277649 | 318.561302 | 367.3139427 |
| ENSMUSG00000035933 | 1.189062904 | 7.63E−07 | 3.47E−05 | 188.465836 | 221.4389538 | 244.2637719 |
| ENSMUSG00000035948 | 1.741049509 | 1.78E−10 | 2.38E−08 | 816.6852892 | 893.5256031 | 857.6780562 |
| ENSMUSG00000036062 | −1.708320866 | 0.004715534 | 0.030261525 | 7.977389882 | 9.712234816 | 3.673139427 |
| ENSMUSG00000036120 | −1.340248384 | 3.85E−09 | 3.64E−07 | 263.2538661 | 219.4965068 | 224.9797899 |
| ENSMUSG00000036611 | 1.04665245 | 4.08E−07 | 2.03E−05 | 846.6005013 | 616.7269108 | 585.8657386 |
| ENSMUSG00000037035 | −1.785829601 | 2.14E−10 | 2.84E−08 | 59.83042412 | 30.10792793 | 50.50566712 |
| ENSMUSG00000037071 | 1.600161226 | 3.89E−11 | 5.71E−09 | 372268.8876 | 280319.3774 | 340820.5063 |
| ENSMUSG00000037095 | −1.027049761 | 7.90E−10 | 8.97E−08 | 3492.102421 | 3177.843232 | 2991.772063 |
| ENSMUSG00000037157 | 1.742314913 | 8.42E−06 | 0.000251859 | 51.85303424 | 60.21585586 | 58.77023083 |
| ENSMUSG00000037336 | −1.65964734 | 0.000936801 | 0.009424872 | 14.95760603 | 7.769787853 | 8.26456371 |
| ENSMUSG00000037443 | −1.349128808 | 2.89E−12 | 5.72E−10 | 750.8718227 | 1078.058065 | 1089.08584 |
| ENSMUSG00000037447 | −1.380120078 | 0.000135203 | 0.002310483 | 30.91238579 | 23.30936356 | 25.71197599 |
| ENSMUSG00000037465 | −1.250556349 | 2.44E−12 | 4.99E−10 | 342.0305912 | 272.9137983 | 297.5242936 |
| ENSMUSG00000037583 | −1.178511493 | 2.71E−06 | 0.000101645 | 181.4856198 | 270.0001279 | 213.9603716 |
| ENSMUSG00000037709 | 1.621036788 | 2.49E−19 | 1.62E−16 | 850.5891962 | 1019.784656 | 857.6780562 |
| ENSMUSG00000037887 | −1.887921138 | 0.000434734 | 0.005464027 | 15.95477976 | 8.741011335 | 11.01941828 |
| ENSMUSG00000038217 | 1.023783748 | 9.74E−10 | 1.07E−07 | 3279.704415 | 3214.749724 | 3049.624009 |
| ENSMUSG00000038233 | 1.132248981 | 2.75E−05 | 0.000651686 | 765.8294287 | 340.8994421 | 856.7597713 |
| ENSMUSG00000038253 | −2.17957789 | 5.49E−08 | 3.76E−06 | 19.94347471 | 18.45324615 | 14.69255771 |
| ENSMUSG00000038370 | −1.863884427 | 6.22E−10 | 7.32E−08 | 95.72867859 | 197.1583668 | 141.4158679 |
| ENSMUSG00000038415 | −1.598903332 | 5.74E−10 | 6.80E−08 | 144.5901916 | 136.9425109 | 205.6958079 |
| ENSMUSG00000038418 | −2.528561862 | 1.50E−17 | 7.58E−15 | 95.72867859 | 79.64032549 | 131.3147345 |
| ENSMUSG00000038473 | 1.076370271 | 0.000625033 | 0.007061263 | 117.6665008 | 63.12952631 | 89.99191596 |
| ENSMUSG00000038530 | −1.722746304 | 4.58E−05 | 0.00098969 | 17.94912724 | 16.51079919 | 13.77427285 |
| ENSMUSG00000038583 | −1.702074711 | 0.00088397 | 0.009002758 | 12.96325856 | 13.59712874 | 11.93770314 |
| ENSMUSG00000038587 | −1.31455808 | 3.12E−06 | 0.000113043 | 97.72302606 | 73.8129846 | 141.4158679 |
| ENSMUSG00000038751 | 1.774904606 | 6.46E−12 | 1.16E−09 | 167.5251875 | 125.2878291 | 171.7192682 |
| ENSMUSG00000038768 | 1.297337324 | 1.64E−05 | 0.000422782 | 329.0673326 | 229.2087417 | 124.8867405 |
| ENSMUSG00000038774 | 1.007283954 | 3.14E−06 | 0.00011311 | 450.7225284 | 497.2664226 | 488.5275438 |
| ENSMUSG00000038844 | 1.003328537 | 1.78E−06 | 7.13E−05 | 364.9655871 | 380.7196048 | 386.5979247 |
| ENSMUSG00000038895 | −1.113485639 | 6.66E−08 | 4.45E−06 | 100.7145473 | 103.9209125 | 94.58334024 |
| ENSMUSG00000039103 | −1.109845105 | 0.002277587 | 0.018174053 | 37.89260194 | 21.3669166 | 26.63026084 |
| ENSMUSG00000039304 | 1.09876453 | 1.10E−06 | 4.76E−05 | 197.4403996 | 176.7626737 | 181.8204016 |
| ENSMUSG00000039533 | 1.597388363 | 4.59E−08 | 3.26E−06 | 367.9571083 | 378.7771578 | 293.8511541 |
| ENSMUSG00000039601 | 1.185885146 | 6.77E−14 | 1.97E−11 | 707.9933521 | 644.8923918 | 732.7913156 |
| ENSMUSG00000039704 | 1.942291849 | 0.00175377 | 0.014994461 | 267.2425611 | 391.4030631 | 463.7338526 |
| ENSMUSG00000039741 | 1.087450189 | 5.12E−08 | 3.58E−06 | 264.2510399 | 270.0001279 | 292.0145844 |
| ENSMUSG00000039853 | 1.374596414 | 3.52E−09 | 3.36E−07 | 477.6462192 | 438.0217902 | 491.2823983 |
| ENSMUSG00000039981 | 1.024216498 | 5.34E−05 | 0.001113305 | 116.669327 | 128.2014996 | 147.8438619 |
| ENSMUSG00000040093 | 1.487860932 | 2.33E−07 | 1.27E−05 | 234.3358278 | 188.4173554 | 357.2128093 |
| ENSMUSG00000040128 | −1.050165381 | 1.95E−11 | 3.16E−09 | 1378.094102 | 1310.180477 | 1381.100424 |
| ENSMUSG00000040152 | −1.254644006 | 0.001007039 | 0.009915514 | 35.89825447 | 19.42446963 | 35.81310941 |
| ENSMUSG00000040435 | −1.205190782 | 4.71E−06 | 0.000155637 | 119.6608482 | 131.11517 | 127.6415951 |
| ENSMUSG00000040584 | 1.185136822 | 2.65E−06 | 9.97E−05 | 302.1436418 | 375.8634874 | 406.8001915 |
| ENSMUSG00000040855 | 1.071792937 | 4.60E−07 | 2.25E−05 | 584.3438089 | 647.8060622 | 645.5542543 |
| ENSMUSG00000040891 | −2.088765162 | 3.05E−41 | 2.08E−37 | 406.846884 | 431.2232258 | 360.8859487 |
| ENSMUSG00000041134 | 1.050155799 | 0.006573499 | 0.03826156 | 41.88129688 | 57.30218542 | 84.48220682 |
| ENSMUSG00000041372 | 2.023614953 | 1.33E−05 | 0.000359411 | 51.85303424 | 49.53239756 | 28.46683056 |
| ENSMUSG00000041695 | −1.532466883 | 9.09E−05 | 0.001683702 | 33.903907 | 18.45324615 | 23.87540627 |
| ENSMUSG00000041702 | 1.159708962 | 1.79E−05 | 0.000451095 | 125.6438906 | 178.7051206 | 179.0655471 |
| ENSMUSG00000041920 | −1.612549242 | 5.78E−11 | 8.29E−09 | 130.6297593 | 116.5468178 | 162.5364196 |
| ENSMUSG00000041930 | −2.069670842 | 7.78E−10 | 8.92E−08 | 29.91521206 | 25.25181052 | 29.38511541 |
| ENSMUSG00000041945 | 2.034436558 | 0.000645414 | 0.007184911 | 35.89825447 | 23.30936356 | 20.20226685 |
| ENSMUSG00000042010 | 1.335313237 | 1.72E−08 | 1.42E−06 | 9652.641758 | 5265.973717 | 7079.05796 |
| ENSMUSG00000042115 | −1.249312452 | 6.34E−07 | 3.01E−05 | 57.83607665 | 47.5899506 | 51.42395197 |
| ENSMUSG00000042246 | −1.458803387 | 0.000333225 | 0.004503661 | 16.9519535 | 19.42446963 | 23.87540627 |
| ENSMUSG00000042333 | 1.17659167 | 0.000212792 | 0.003210009 | 83.76259377 | 76.72665505 | 64.27993997 |
| ENSMUSG00000042354 | −1.004338102 | 0.000195353 | 0.003017019 | 312.1153791 | 284.5684801 | 269.057463 |
| ENSMUSG00000042379 | −1.128744357 | 0.001182685 | 0.011115132 | 63.81911906 | 39.82016275 | 89.99191596 |
| ENSMUSG00000042444 | 1.165704626 | 4.84E−10 | 5.85E−08 | 592.3211988 | 520.5757862 | 674.0210848 |
| ENSMUSG00000042510 | −1.154445577 | 0.002024484 | 0.016651324 | 28.91803832 | 29.13670445 | 26.63026084 |
| ENSMUSG00000042607 | −1.584091426 | 0.001074406 | 0.010435667 | 12.96325856 | 6.798564371 | 14.69255771 |
| ENSMUSG00000042622 | −1.343729967 | 0.000106867 | 0.001912519 | 29.91521206 | 22.33814008 | 31.22168513 |
| ENSMUSG00000042680 | 1.086900564 | 0.000262276 | 0.00374505 | 262.2566924 | 328.2735368 | 358.1310941 |
| ENSMUSG00000042743 | 1.143476031 | 0.005459928 | 0.033421057 | 39.88694941 | 35.93526882 | 50.50566712 |
| ENSMUSG00000042745 | −1.243661764 | 2.94E−08 | 2.25E−06 | 425.793185 | 215.6116129 | 317.7265604 |
| ENSMUSG00000043165 | −1.65740279 | 0.002445867 | 0.019147761 | 6.980216147 | 13.59712874 | 9.182848567 |
| ENSMUSG00000043421 | −1.515224307 | 3.07E−05 | 0.000711553 | 31.90955953 | 28.16548097 | 33.9765397 |
| ENSMUSG00000043639 | 1.042566076 | 0.00209562 | 0.017092091 | 83.76259377 | 70.89931416 | 87.23706139 |
| ENSMUSG00000043681 | −1.124534949 | 2.43E−09 | 2.42E−07 | 201.4290945 | 238.9209765 | 202.0226685 |
| ENSMUSG00000044042 | 1.154056163 | 3.06E−05 | 0.000711225 | 258.2679974 | 274.8562453 | 353.5396698 |
| ENSMUSG00000044186 | 1.148916053 | 0.009243418 | 0.048746497 | 35.89825447 | 33.99282186 | 30.30340027 |
| ENSMUSG00000044339 | −1.202694639 | 0.000240284 | 0.0035008 | 44.87281809 | 27.19425749 | 28.46683056 |
| ENSMUSG00000044349 | 1.020247724 | 0.000864482 | 0.008883909 | 170.5167087 | 218.5252834 | 182.7386865 |
| ENSMUSG00000044359 | 1.419594491 | 2.01E−07 | 1.12E−05 | 221.3725692 | 303.9929497 | 263.5477539 |
| ENSMUSG00000044676 | −1.235532178 | 3.06E−06 | 0.000111153 | 173.5082299 | 206.8706016 | 146.0072922 |
| ENSMUSG00000044749 | 1.260739468 | 0.003720434 | 0.025546608 | 2581.682801 | 3424.533996 | 3631.816608 |
| ENSMUSG00000044948 | −1.380324526 | 9.28E−05 | 0.001711935 | 28.91803832 | 18.45324615 | 23.87540627 |
| ENSMUSG00000045045 | 1.563883127 | 9.68E−08 | 6.14E−06 | 106.6975897 | 117.5180413 | 171.7192682 |
| ENSMUSG00000045294 | 1.216661032 | 2.49E−17 | 1.21E−14 | 16820.32657 | 15644.46784 | 18334.47545 |
| ENSMUSG00000045348 | −1.535176629 | 0.002140436 | 0.017319005 | 17.94912724 | 8.741011335 | 9.182848567 |
| ENSMUSG00000045382 | −1.705364762 | 8.30E−05 | 0.001575106 | 17.94912724 | 15.53957571 | 23.87540627 |
| ENSMUSG00000045411 | −1.265704961 | 6.98E−06 | 0.000215157 | 217.3838743 | 180.6475676 | 227.7346445 |
| ENSMUSG00000045776 | 1.435732199 | 4.30E−15 | 1.59E−12 | 1029.083295 | 1166.439401 | 1214.890865 |
| ENSMUSG00000045875 | 1.205673082 | 0.000172777 | 0.002749319 | 91.73998365 | 131.11517 | 117.5404617 |
| ENSMUSG00000046541 | 1.523461987 | 5.84E−06 | 0.000185032 | 69.80216147 | 95.1799012 | 84.48220682 |
| ENSMUSG00000046721 | −1.016110295 | 0.006334733 | 0.037187581 | 17.94912724 | 26.223034 | 22.95712142 |
| ENSMUSG00000046908 | −1.354651164 | 0.005396039 | 0.033236578 | 15.95477976 | 8.741011335 | 10.10113342 |
| ENSMUSG00000047496 | 1.093888518 | 1.32E−05 | 0.00035939 | 433.7705749 | 520.5757862 | 451.7961495 |
| ENSMUSG00000047649 | −1.227032883 | 5.45E−06 | 0.000175137 | 72.79368268 | 67.01442023 | 64.27993997 |
| ENSMUSG00000047875 | 1.019701978 | 0.000156296 | 0.002567963 | 101.711721 | 104.892136 | 85.40049167 |
| ENSMUSG00000048191 | −1.584188448 | 0.000509157 | 0.006122909 | 10.96891109 | 18.45324615 | 11.01941828 |
| ENSMUSG00000048644 | −2.138371107 | 3.35E−05 | 0.00076585 | 5.983042412 | 8.741011335 | 8.26456371 |
| ENSMUSG00000048856 | −1.529839383 | 8.95E−23 | 8.72E−20 | 3855.073661 | 3458.526818 | 4005.558545 |
| ENSMUSG00000049044 | −1.523296713 | 7.27E−17 | 3.42E−14 | 783.7785559 | 1199.461 | 1266.314817 |
| ENSMUSG00000049313 | 1.020089742 | 3.99E−06 | 0.000136353 | 328.0701589 | 236.9785295 | 312.2168513 |
| ENSMUSG00000049580 | −1.860685334 | 2.05E−30 | 5.59E−27 | 548.4455544 | 407.9138623 | 479.3446952 |
| ENSMUSG00000049791 | 1.116973741 | 1.01E−06 | 4.43E−05 | 636.1968431 | 678.8852137 | 759.4215765 |
| ENSMUSG00000049950 | −1.164244145 | 0.007174451 | 0.040867998 | 18.94630097 | 37.87771578 | 21.1205517 |
| ENSMUSG00000050390 | 1.148547066 | 4.80E−15 | 1.72E−12 | 1574.537328 | 1571.439593 | 1292.026793 |
| ENSMUSG00000050503 | −1.747508686 | 0.001612805 | 0.014098606 | 6.980216147 | 9.712234816 | 7.346278854 |
| ENSMUSG00000050663 | 1.366962359 | 0.003061462 | 0.022321797 | 20.94064844 | 51.47484453 | 44.07767312 |
| ENSMUSG00000050737 | −1.262831751 | 0.008424555 | 0.045643887 | 26.92369085 | 13.59712874 | 44.99595798 |
| ENSMUSG00000050914 | −2.867341906 | 0.000724516 | 0.007829023 | 13.96043229 | 23.30936356 | 14.69255771 |
| ENSMUSG00000051149 | 1.736018564 | 0.001879809 | 0.015833824 | 31.90955953 | 33.02159838 | 39.48624884 |
| ENSMUSG00000051339 | 1.051356464 | 2.17E−16 | 9.53E−14 | 1923.548135 | 1976.439785 | 1850.343986 |
| ENSMUSG00000051452 | 1.412708477 | 1.55E−10 | 2.11E−08 | 276.2171247 | 240.8634234 | 283.7500207 |
| ENSMUSG00000051674 | 1.054513908 | 9.53E−09 | 8.39E−07 | 515.5388212 | 743.9571869 | 608.82286 |
| ENSMUSG00000051998 | −1.5364341 | 0.002646663 | 0.020230437 | 9.971737353 | 19.42446963 | 6.427993997 |
| ENSMUSG00000052085 | 1.272820714 | 3.30E−11 | 4.99E−09 | 423.7988375 | 546.7988202 | 446.2864404 |
| ENSMUSG00000052595 | 1.233431784 | 0.003777269 | 0.025781088 | 1666.277312 | 2467.878867 | 2847.601341 |
| ENSMUSG00000052656 | 1.063492715 | 4.09E−12 | 7.63E−10 | 4427.451385 | 3448.814583 | 3382.043127 |
| ENSMUSG00000052684 | −1.26752533 | 1.09E−07 | 6.78E−06 | 161.5421451 | 161.223098 | 181.8204016 |
| ENSMUSG00000052713 | 1.631079765 | 2.29E−06 | 8.94E−05 | 74.78803015 | 84.4964429 | 102.8479039 |
| ENSMUSG00000052837 | −1.91589408 | 1.98E−18 | 1.08E−15 | 205.4177895 | 302.0505028 | 276.4037419 |
| ENSMUSG00000053560 | −1.145431158 | 4.41E−10 | 5.44E−08 | 324.081464 | 268.0576809 | 242.4272022 |
| ENSMUSG00000053964 | −1.860738134 | 2.78E−14 | 8.62E−12 | 100.7145473 | 119.4604882 | 153.3535711 |
| ENSMUSG00000053977 | −1.428502316 | 0.007562752 | 0.042337131 | 8.974563618 | 28.16548097 | 7.346278854 |
| ENSMUSG00000054008 | 1.359856014 | 0.005424595 | 0.033352211 | 3190.955953 | 3047.699285 | 3392.144261 |
| ENSMUSG00000054150 | 1.583267643 | 1.23E−07 | 7.44E−06 | 98.72019979 | 105.8633595 | 78.97249768 |
| ENSMUSG00000054422 | −1.006699851 | 5.17E−07 | 2.51E−05 | 21378.40771 | 26089.97639 | 27702.81756 |
| ENSMUSG00000054453 | 1.090734081 | 0.000462815 | 0.005711685 | 93.73433112 | 87.41011335 | 126.7233102 |
| ENSMUSG00000054659 | 1.244126007 | 0.002259781 | 0.018053098 | 79.77389882 | 73.8129846 | 100.0930494 |
| ENSMUSG00000054932 | 1.219377704 | 9.42E−05 | 0.001724207 | 87.75128871 | 202.0144842 | 233.2443536 |
| ENSMUSG00000055148 | −1.210391107 | 1.04E−07 | 6.56E−06 | 210.4036581 | 153.4533101 | 137.7427285 |
| ENSMUSG00000055254 | 1.702350158 | 1.36E−13 | 3.56E−11 | 1260.427601 | 1100.396205 | 2139.603716 |
| ENSMUSG00000055491 | −1.429157403 | 5.36E−08 | 3.69E−06 | 191.4573572 | 205.8993781 | 144.1707225 |
| ENSMUSG00000055660 | 1.21539125 | 9.47E−05 | 0.001725945 | 86.75411497 | 97.12234816 | 112.0307525 |
| ENSMUSG00000055692 | −1.507668302 | 6.91E−05 | 0.001367589 | 23.93216965 | 24.28058704 | 19.28398199 |
| ENSMUSG00000055980 | 1.007478258 | 8.74E−05 | 0.001638705 | 336.0475488 | 377.8059344 | 381.0882155 |
| ENSMUSG00000056054 | −4.00774487 | 1.39E−20 | 1.11E−17 | 6.980216147 | 6.798564371 | 4.591424283 |
| ENSMUSG00000056071 | −3.639131514 | 1.15E−24 | 1.74E−21 | 15.95477976 | 8.741011335 | 12.85598799 |
| ENSMUSG00000056091 | −1.031452251 | 1.62E−09 | 1.68E−07 | 1990.358776 | 1481.115809 | 2190.109383 |
| ENSMUSG00000056148 | 1.3596028 | 0.000119043 | 0.002094688 | 75.78520388 | 108.7770299 | 71.62621882 |
| ENSMUSG00000056313 | −1.726808914 | 2.85E−11 | 4.37E−09 | 435.7649223 | 244.7483174 | 415.0647552 |
| ENSMUSG00000057342 | 1.09304789 | 8.77E−15 | 2.92E−12 | 1269.402165 | 1384.964685 | 1236.011417 |
| ENSMUSG00000057604 | −1.272122676 | 0.008168023 | 0.044709526 | 10.96891109 | 10.6834583 | 11.93770314 |
| ENSMUSG00000057722 | −1.840565518 | 1.78E−07 | 1.02E−05 | 45.86999182 | 35.93526882 | 110.1941828 |
| ENSMUSG00000057969 | 1.066228289 | 0.000219235 | 0.003267437 | 95.72867859 | 74.78420809 | 99.17476452 |
| ENSMUSG00000058207 | −1.338541988 | 1.08E−13 | 2.88E−11 | 23432.58561 | 23413.28447 | 28330.9244 |
| ENSMUSG00000058503 | −1.136818503 | 6.67E−07 | 3.10E−05 | 130.6297593 | 164.1367684 | 153.3535711 |
| ENSMUSG00000058793 | 1.056167061 | 4.57E−10 | 5.57E−08 | 3494.096768 | 3071.008649 | 3164.409616 |
| ENSMUSG00000058794 | 1.61977926 | 2.23E−06 | 8.73E−05 | 144.5901916 | 102.9496891 | 144.1707225 |
| ENSMUSG00000058921 | 1.372756574 | 4.51E−07 | 2.22E−05 | 911.4167941 | 1126.619239 | 1263.559963 |
| ENSMUSG00000059149 | 1.084964814 | 0.00015449 | 0.002547498 | 343.0277649 | 296.2231619 | 348.9482455 |
| ENSMUSG00000059824 | 4.510710868 | 5.51E−13 | 1.32E−10 | 248.2962601 | 730.3600582 | 248.8551962 |
| ENSMUSG00000060429 | 1.717674566 | 1.22E−12 | 2.68E−10 | 734.9170429 | 831.3673003 | 944.9151175 |
| ENSMUSG00000061175 | 1.152852085 | 3.29E−14 | 9.98E−12 | 1100.879804 | 976.079599 | 989.9110755 |
| ENSMUSG00000061292 | 1.112980568 | 1.04E−05 | 0.000296719 | 1260.427601 | 1520.935972 | 1350.797024 |
| ENSMUSG00000061436 | 1.042732241 | 2.40E−05 | 0.000578522 | 326.0758114 | 431.2232258 | 663.9199514 |
| ENSMUSG00000061536 | 1.045389319 | 1.00E−05 | 0.000287463 | 219.3782218 | 260.2878931 | 199.2678139 |
| ENSMUSG00000061825 | 1.826944112 | 9.41E−14 | 2.63E−11 | 718.9622632 | 690.5398954 | 678.6125091 |
| ENSMUSG00000062901 | 1.319446488 | 6.22E−09 | 5.70E−07 | 788.7644246 | 893.5256031 | 992.6659301 |
| ENSMUSG00000063535 | 1.201850675 | 0.000151738 | 0.002508185 | 62.82194532 | 71.87053764 | 70.70793397 |
| ENSMUSG00000063704 | −1.127471271 | 2.47E−07 | 1.32E−05 | 294.1662519 | 311.7627376 | 224.061505 |
| ENSMUSG00000063929 | −1.198836957 | 5.83E−06 | 0.000185032 | 50.8558605 | 105.8633595 | 82.6456371 |
| ENSMUSG00000065126 | −1.713055356 | 1.38E−07 | 8.18E−06 | 29.91521206 | 48.56117408 | 40.40453369 |
| ENSMUSG00000065147 | −1.294541943 | 0.007255788 | 0.041125179 | 14.95760603 | 12.62590526 | 15.61084256 |
| ENSMUSG00000065952 | 1.57267976 | 1.62E−10 | 2.19E−08 | 199.4347471 | 157.338204 | 188.2483956 |
| ENSMUSG00000066456 | 1.032022635 | 0.005405213 | 0.033278057 | 76.78237762 | 64.10074979 | 150.5987165 |
| ENSMUSG00000066477 | −1.128543552 | 2.29E−05 | 0.000561201 | 57.83607665 | 44.67628015 | 49.58738226 |
| ENSMUSG00000066687 | −2.591322343 | 5.98E−23 | 6.28E−20 | 139.6043229 | 122.3741587 | 161.6181348 |
| ENSMUSG00000066944 | 1.544707207 | 0.000412457 | 0.005261624 | 43.87564435 | 49.53239756 | 40.40453369 |
| ENSMUSG00000067149 | −1.843573184 | 0.000282252 | 0.003980428 | 113.6778058 | 169.9641093 | 162.5364196 |
| ENSMUSG00000068463 | −2.382320454 | 0.000472976 | 0.005821273 | 4.985868677 | 3.884893927 | 5.50970914 |
| ENSMUSG00000068742 | 1.190690012 | 3.19E−12 | 6.14E−10 | 611.2674997 | 770.1802209 | 691.4684971 |
| ENSMUSG00000068877 | −1.095647878 | 6.50E−07 | 3.04E−05 | 98.72019979 | 98.09357164 | 136.8244436 |
| ENSMUSG00000069456 | 1.727916701 | 1.48E−26 | 2.89E−23 | 6191.451722 | 5969.139518 | 5731.934075 |
| ENSMUSG00000069804 | −2.067148134 | 1.72E−05 | 0.000437213 | 3.988694941 | 13.59712874 | 13.77427285 |
| ENSMUSG00000070576 | 1.095801322 | 1.06E−07 | 6.63E−06 | 253.2821288 | 274.8562453 | 226.8163596 |
| ENSMUSG00000070583 | 1.188428844 | 0.006226465 | 0.036681868 | 41.88129688 | 47.5899506 | 40.40453369 |
| ENSMUSG00000071076 | 1.243260003 | 3.56E−08 | 2.62E−06 | 1324.24672 | 1340.288405 | 938.4871235 |
| ENSMUSG00000071456 | 1.345823726 | 0.001263422 | 0.011712633 | 45.86999182 | 70.89931416 | 48.6690974 |
| ENSMUSG00000071547 | −1.168738292 | 0.00025901 | 0.003706323 | 44.87281809 | 25.25181052 | 47.75081255 |
| ENSMUSG00000071637 | −1.028910519 | 0.000250761 | 0.0036106 | 107.6947634 | 64.10074979 | 89.99191596 |
| ENSMUSG00000071645 | −1.14015686 | 5.54E−07 | 2.68E−05 | 206.4149632 | 273.8850218 | 188.2483956 |
| ENSMUSG00000072294 | 2.084673623 | 3.53E−08 | 2.62E−06 | 97.72302606 | 148.5971927 | 196.5129593 |
| ENSMUSG00000072571 | 1.01455077 | 0.003907572 | 0.026379983 | 56.83890291 | 47.5899506 | 55.0970914 |
| ENSMUSG00000072664 | 1.275253331 | 1.57E−06 | 6.41E−05 | 951.3037435 | 1401.475484 | 1131.326943 |
| ENSMUSG00000072692 | −1.135811828 | 0.000365333 | 0.004818223 | 34.90108074 | 29.13670445 | 28.46683056 |
| ENSMUSG00000072849 | −2.106422019 | 7.14E−21 | 6.08E−18 | 100.7145473 | 96.15112468 | 102.8479039 |
| ENSMUSG00000072999 | −1.145851135 | 1.86E−07 | 1.05E−05 | 116.669327 | 176.7626737 | 168.0461288 |
| ENSMUSG00000073460 | −1.28577089 | 0.005211876 | 0.032439227 | 18.94630097 | 39.82016275 | 10.10113342 |
| ENSMUSG00000073835 | 2.295482419 | 0.000631649 | 0.007089542 | 332.0588539 | 185.503685 | 325.0728393 |
| ENSMUSG00000074024 | −1.08003527 | 4.28E−05 | 0.000939631 | 43.87564435 | 61.18707934 | 53.26052169 |
| ENSMUSG00000074063 | −2.210318446 | 1.23E−07 | 7.44E−06 | 989.1963454 | 819.7126185 | 1493.131177 |
| ENSMUSG00000074213 | 1.225081814 | 0.003513884 | 0.024460869 | 87.75128871 | 38.84893927 | 37.64967912 |
| ENSMUSG00000074345 | 1.610694413 | 1.86E−06 | 7.43E−05 | 92.73715738 | 95.1799012 | 56.93366111 |
| ENSMUSG00000074375 | 1.123374183 | 1.71E−16 | 7.76E−14 | 1739.070994 | 2059.965005 | 1850.343986 |
| ENSMUSG00000074876 | 1.013446872 | 0.000205306 | 0.003120937 | 101.711721 | 119.4604882 | 117.5404617 |
| ENSMUSG00000075470 | 1.495450545 | 0.008033121 | 0.044154642 | 346.0192861 | 387.5181692 | 422.4110341 |
| ENSMUSG00000075552 | 1.02486804 | 2.18E−07 | 1.20E−05 | 1632.373405 | 1558.813688 | 2640.068963 |
| ENSMUSG00000075590 | −1.36961476 | 1.11E−14 | 3.51E−12 | 556.4229443 | 571.0794072 | 507.8115258 |
| ENSMUSG00000076490 | −1.690670704 | 0.002848332 | 0.021272015 | 9.971737353 | 4.856117408 | 8.26456371 |
| ENSMUSG00000076569 | −1.340585852 | 0.000314538 | 0.004311858 | 26.92369085 | 70.89931416 | 46.83252769 |
| ENSMUSG00000076596 | −3.365323343 | 1.13E−07 | 6.96E−06 | 3.988694941 | 6.798564371 | 9.182848567 |
| ENSMUSG00000076609 | −2.421594655 | 3.22E−06 | 0.000115075 | 484.6264354 | 675.0003197 | 704.3244851 |
| ENSMUSG00000076613 | −1.082440472 | 0.000721125 | 0.007817157 | 31.90955953 | 61.18707934 | 74.38107339 |
| ENSMUSG00000076617 | −1.218011643 | 6.44E−08 | 4.32E−06 | 1755.025774 | 1885.144778 | 1859.526835 |
| ENSMUSG00000076934 | −3.037010136 | 1.44E−09 | 1.52E−07 | 7.977389882 | 2.913670445 | 10.10113342 |
| ENSMUSG00000077148 | 1.938039708 | 8.47E−06 | 0.000252598 | 48.86151303 | 38.84893927 | 69.78964911 |
| ENSMUSG00000078193 | −1.269506646 | 0.004582659 | 0.029506003 | 13.96043229 | 9.712234816 | 21.1205517 |
| ENSMUSG00000078234 | 1.031251449 | 6.47E−07 | 3.04E−05 | 557.420118 | 503.0937635 | 599.6400114 |
| ENSMUSG00000078650 | −1.017019231 | 3.99E−07 | 2.00E−05 | 5816.514398 | 3628.490927 | 4180.950953 |
| ENSMUSG00000078651 | −2.099102297 | 6.20E−07 | 2.95E−05 | 8.974563618 | 10.6834583 | 16.52912742 |
| ENSMUSG00000078672 | −1.051061583 | 1.66E−05 | 0.000428362 | 108.6919371 | 197.1583668 | 152.4352862 |
| ENSMUSG00000078688 | 1.787709931 | 3.76E−11 | 5.58E−09 | 119.6608482 | 143.7410753 | 173.5558379 |
| ENSMUSG00000078817 | −1.628755428 | 1.37E−11 | 2.31E−09 | 107.6947634 | 79.64032549 | 80.80906739 |
| ENSMUSG00000079017 | −1.001509959 | 0.000541038 | 0.006404635 | 74.78803015 | 60.21585586 | 44.07767312 |
| ENSMUSG00000079036 | −1.378588057 | 0.008915895 | 0.047568881 | 170.5167087 | 124.3166056 | 106.5210434 |
| ENSMUSG00000079065 | −1.153356662 | 4.39E−06 | 0.000146637 | 123.6495432 | 164.1367684 | 125.8050254 |
| ENSMUSG00000079465 | −1.030415111 | 0.006780187 | 0.03914539 | 18.94630097 | 27.19425749 | 22.03883656 |
| ENSMUSG00000079470 | 1.222302248 | 2.09E−11 | 3.33E−09 | 498.5868677 | 463.2736007 | 474.7532709 |
| ENSMUSG00000080059 | −1.546208951 | 0.008392591 | 0.045561211 | 7.977389882 | 9.712234816 | 6.427993997 |
| ENSMUSG00000081344 | −1.007584184 | 0.007335419 | 0.041406148 | 22.93499591 | 19.42446963 | 38.56796398 |
| ENSMUSG00000082065 | 2.441656125 | 0.003600075 | 0.02496215 | 179.4912724 | 141.7986283 | 255.2831902 |
| ENSMUSG00000082173 | 2.591199516 | 2.10E−11 | 3.33E−09 | 150.573234 | 114.6043708 | 224.061505 |
| ENSMUSG00000082586 | 1.935822229 | 3.52E−06 | 0.000123572 | 53.84738171 | 55.35973845 | 47.75081255 |
| ENSMUSG00000082658 | −1.204694651 | 0.004700851 | 0.030195718 | 67.807814 | 23.30936356 | 23.87540627 |
| ENSMUSG00000083327 | 1.031096879 | 0.000937166 | 0.009424872 | 102.7088947 | 96.15112468 | 82.6456371 |
| ENSMUSG00000083621 | −1.163358375 | 0.003315291 | 0.023547198 | 19.94347471 | 19.42446963 | 20.20226685 |
| ENSMUSG00000083716 | −1.168157812 | 0.001666635 | 0.01443041 | 25.92651712 | 25.25181052 | 35.81310941 |
| ENSMUSG00000083813 | −1.588176086 | 8.35E−05 | 0.001580916 | 11.96608482 | 53.41729149 | 22.03883656 |
| ENSMUSG00000083863 | −1.215047324 | 0.000627696 | 0.007061263 | 24.92934338 | 19.42446963 | 27.5485457 |
| ENSMUSG00000083992 | −1.283026783 | 0.003457083 | 0.024176532 | 11.96608482 | 12.62590526 | 20.20226685 |
| ENSMUSG00000084822 | −1.954137616 | 1.52E−07 | 8.88E−06 | 16.9519535 | 25.25181052 | 13.77427285 |
| ENSMUSG00000084883 | 1.839966406 | 9.14E−09 | 8.09E−07 | 157.5534502 | 141.7986283 | 126.7233102 |
| ENSMUSG00000085001 | −2.14213554 | 3.42E−08 | 2.56E−06 | 29.91521206 | 16.51079919 | 21.1205517 |
| ENSMUSG00000085156 | −1.793249778 | 0.00014396 | 0.002408808 | 24.92934338 | 31.07915141 | 16.52912742 |
| ENSMUSG00000085445 | −1.469856228 | 8.55E−08 | 5.52E−06 | 45.86999182 | 46.61872712 | 72.54450368 |
| ENSMUSG00000085834 | −3.692490083 | 4.07E−39 | 1.85E−35 | 50.8558605 | 56.33096193 | 71.62621882 |
| ENSMUSG00000085995 | −1.230019641 | 9.11E−06 | 0.000268289 | 189.4630097 | 167.0504388 | 353.5396698 |
| ENSMUSG00000086140 | 1.216209638 | 0.003436989 | 0.024085413 | 34.90108074 | 60.21585586 | 44.99595798 |
| ENSMUSG00000086446 | 2.158985376 | 0.000113634 | 0.002012508 | 17.94912724 | 6.798564371 | 9.182848567 |
| ENSMUSG00000086529 | 2.155075918 | 4.95E−05 | 0.001053778 | 27.92086459 | 38.84893927 | 44.07767312 |
| ENSMUSG00000086786 | 1.909493382 | 0.004138057 | 0.027366965 | 31.90955953 | 14.56835222 | 33.05825484 |
| ENSMUSG00000086844 | −1.797314635 | 1.20E−05 | 0.000331862 | 10.96891109 | 17.48202267 | 18.36569713 |
| ENSMUSG00000087382 | −1.074896574 | 1.10E−05 | 0.000308734 | 161.5421451 | 285.5397036 | 136.8244436 |
| ENSMUSG00000087445 | −1.629934605 | 4.98E−05 | 0.001058506 | 21.93782218 | 12.62590526 | 15.61084256 |
| ENSMUSG00000087595 | −1.356715257 | 0.000782501 | 0.008272069 | 15.95477976 | 21.3669166 | 17.44741228 |
| ENSMUSG00000087613 | 1.300460925 | 0.002649524 | 0.020230437 | 53.84738171 | 60.21585586 | 82.6456371 |
| ENSMUSG00000087616 | −2.549514093 | 7.92E−06 | 0.000238545 | 14.95760603 | 13.59712874 | 8.26456371 |
| ENSMUSG00000087658 | −1.652423913 | 0.001338341 | 0.012240744 | 12.96325856 | 5.82734089 | 9.182848567 |
| ENSMUSG00000089726 | −1.506918387 | 4.30E−05 | 0.000942053 | 73.79085641 | 59.24463238 | 45.91424283 |
| ENSMUSG00000089943 | 1.766690487 | 7.03E−13 | 1.62E−10 | 3075.2838 | 2604.821378 | 3147.880489 |
| ENSMUSG00000090021 | −1.656392969 | 0.002988368 | 0.021957099 | 5.983042412 | 7.769787853 | 19.28398199 |
| ENSMUSG00000090145 | 1.252091278 | 1.05E−06 | 4.56E−05 | 514.5416474 | 669.1729788 | 606.0680054 |
| ENSMUSG00000090175 | 1.660039728 | 0.006419999 | 0.037574902 | 840.6174589 | 1098.453758 | 415.0647552 |
| ENSMUSG00000090264 | −2.18334921 | 1.38E−07 | 8.18E−06 | 7.977389882 | 12.62590526 | 21.1205517 |
| ENSMUSG00000090369 | 1.713925914 | 3.56E−08 | 2.62E−06 | 82.76542003 | 126.2590526 | 106.5210434 |
| ENSMUSG00000090555 | −2.901973235 | 9.10E−10 | 1.01E−07 | 121.6551957 | 110.7194769 | 103.7661888 |
| ENSMUSG00000090610 | 1.076721434 | 0.00270073 | 0.020438322 | 79.77389882 | 53.41729149 | 47.75081255 |
| ENSMUSG00000090698 | −2.55687976 | 1.21E−08 | 1.04E−06 | 20.94064844 | 19.42446963 | 24.79369113 |
| ENSMUSG00000091021 | −1.527245218 | 0.003008341 | 0.022044462 | 10.96891109 | 16.51079919 | 18.36569713 |
| ENSMUSG00000091509 | −1.002937779 | 0.000805178 | 0.008420412 | 69.80216147 | 64.10074979 | 68.87136425 |
| ENSMUSG00000092075 | −5.716673555 | 4.92E−16 | 1.97E−13 | 2.991521206 | 2.913670445 | 0 |
| ENSMUSG00000094410 | 1.220382244 | 0.001002234 | 0.009882478 | 53.84738171 | 132.0863935 | 116.6221768 |
| ENSMUSG00000095280 | −1.650205335 | 4.03E−05 | 0.000893784 | 58.83325038 | 25.25181052 | 24.79369113 |
| ENSMUSG00000095351 | −3.092804449 | 0.006028299 | 0.035789253 | 6.980216147 | 60.21585586 | 166.2095591 |
| ENSMUSG00000096833 | 3.208484861 | 0.004128622 | 0.02736411 | 10.96891109 | 124.3166056 | 23.87540627 |
| ENSMUSG00000096910 | 1.124675865 | 0.006036 | 0.035819378 | 64.81629279 | 67.01442023 | 72.54450368 |
| ENSMUSG00000096954 | 1.206343759 | 0.003120612 | 0.022529545 | 33.903907 | 87.41011335 | 81.72735225 |
| ENSMUSG00000097124 | 2.16107918 | 3.03E−13 | 7.50E−11 | 133.6212805 | 103.9209125 | 130.3964497 |
| ENSMUSG00000097221 | 1.550011559 | 0.000809985 | 0.008457711 | 46.86716556 | 44.67628015 | 42.24110341 |
| ENSMUSG00000097312 | −1.433041605 | 0.000236458 | 0.003456142 | 49.85868677 | 34.96404534 | 33.9765397 |
| ENSMUSG00000097536 | 1.179506676 | 0.003789911 | 0.025815692 | 36.89542821 | 55.35973845 | 40.40453369 |
| ENSMUSG00000097615 | −1.278009058 | 7.24E−05 | 0.001413729 | 33.903907 | 25.25181052 | 25.71197599 |
| ENSMUSG00000097660 | −2.932543755 | 3.83E−06 | 0.000131806 | 4.985868677 | 4.856117408 | 6.427993997 |
| ENSMUSG00000097691 | 1.274551418 | 4.53E−08 | 3.23E−06 | 235.3330015 | 259.3166696 | 175.3924076 |
| ENSMUSG00000097743 | −1.078551643 | 0.000185309 | 0.002900208 | 55.84172918 | 55.35973845 | 50.50566712 |
| ENSMUSG00000097908 | 1.156232879 | 5.54E−05 | 0.001144881 | 154.561929 | 95.1799012 | 98.25647967 |
| ENSMUSG00000097971 | −1.77227667 | 3.92E−13 | 9.56E−11 | 2017.282467 | 2103.670061 | 1313.147345 |
| ENSMUSG00000097994 | −1.156597047 | 0.008206265 | 0.04482173 | 14.95760603 | 25.25181052 | 11.93770314 |
| ENSMUSG00000098041 | −1.469996023 | 0.000332181 | 0.004498467 | 22.93499591 | 20.39569311 | 17.44741228 |
| ENSMUSG00000098661 | −1.627002493 | 0.000277688 | 0.003922434 | 19.94347471 | 6.798564371 | 14.69255771 |
| ENSMUSG00000098814 | −4.705165719 | 0.001128634 | 0.010794205 | 2.991521206 | 1.942446963 | 1.836569713 |
| ENSMUSG00000098882 | 1.905068974 | 0.000139968 | 0.002360908 | 128.6354119 | 27.19425749 | 93.66505538 |
| ENSMUSG00000099568 | 2.251364762 | 0.000320113 | 0.004365382 | 1.994347471 | 4.856117408 | 19.28398199 |
| ENSMUSG00000099858 | 1.088333588 | 4.80E−05 | 0.001024996 | 154.561929 | 202.0144842 | 168.0461288 |
| ENSMUSG00000100094 | 1.758512646 | 5.46E−23 | 6.28E−20 | 3175.001173 | 2305.684545 | 3195.631301 |
| ENSMUSG00000100468 | 1.995883006 | 0.000292618 | 0.004105376 | 17.94912724 | 36.9064923 | 34.89482455 |
| ENSMUSG00000101939 | −1.264629649 | 0.000784437 | 0.008279693 | 14.95760603 | 28.16548097 | 21.1205517 |
| ENSMUSG00000102275 | −1.950030022 | 0.000225327 | 0.003321924 | 4.985868677 | 13.59712874 | 14.69255771 |
| ENSMUSG00000102577 | −1.649520189 | 1.70E−08 | 1.42E−06 | 40.88412315 | 64.10074979 | 82.6456371 |
| ENSMUSG00000102719 | −1.730894623 | 0.006769906 | 0.039102589 | 12.96325856 | 5.82734089 | 4.591424283 |
| ENSMUSG00000102869 | 1.362067771 | 5.85E−06 | 0.000185032 | 292.1719044 | 335.0721012 | 361.8042335 |
| ENSMUSG00000102882 | 2.19227753 | 3.68E−07 | 1.87E−05 | 88.74846244 | 63.12952631 | 216.7152262 |
| ENSMUSG00000102918 | 1.26420677 | 0.002814898 | 0.021114835 | 30.91238579 | 47.5899506 | 66.11650968 |
| ENSMUSG00000103285 | −1.692405475 | 0.00269655 | 0.020438322 | 9.971737353 | 5.82734089 | 8.26456371 |
| ENSMUSG00000103546 | −1.97976669 | 0.000414043 | 0.005267075 | 6.980216147 | 6.798564371 | 6.427993997 |
| ENSMUSG00000104030 | −3.028455317 | 2.18E−07 | 1.20E−05 | 3.988694941 | 4.856117408 | 5.50970914 |
| ENSMUSG00000104388 | −2.61061807 | 5.15E−05 | 0.001078975 | 6.980216147 | 1.942446963 | 6.427993997 |
| ENSMUSG00000104399 | −1.177892321 | 0.00272173 | 0.020574411 | 23.93216965 | 29.13670445 | 18.36569713 |
| ENSMUSG00000104445 | 1.012908858 | 0.003001159 | 0.022015494 | 68.80498774 | 51.47484453 | 89.99191596 |
| ENSMUSG00000104973 | −2.007621104 | 9.36E−05 | 0.001719883 | 6.980216147 | 13.59712874 | 4.591424283 |
| ENSMUSG00000105161 | −1.279752452 | 0.00691512 | 0.039789659 | 21.93782218 | 8.741011335 | 13.77427285 |
| ENSMUSG00000105434 | −2.02241612 | 0.000742647 | 0.007975668 | 5.983042412 | 7.769787853 | 6.427993997 |
| ENSMUSG00000105547 | −2.707072316 | 4.65E−06 | 0.000153843 | 5.983042412 | 2.913670445 | 8.26456371 |
| ENSMUSG00000105556 | 1.504518371 | 0.00309422 | 0.022420764 | 21.93782218 | 29.13670445 | 33.05825484 |
| ENSMUSG00000105703 | −1.801332207 | 3.38E−12 | 6.40E−10 | 242.3132177 | 122.3741587 | 160.6998499 |
| ENSMUSG00000105881 | 1.259909682 | 7.19E−05 | 0.001410701 | 247.2990864 | 245.7195409 | 252.5283356 |
| ENSMUSG00000105906 | −2.671616891 | 0.003078263 | 0.022366209 | 6.980216147 | 20.39569311 | 14.69255771 |
| ENSMUSG00000106030 | 1.145150269 | 5.54E−05 | 0.001144881 | 109.6891109 | 147.6259692 | 198.349529 |
| ENSMUSG00000106664 | −1.606191029 | 0.000152255 | 0.002513686 | 9.971737353 | 17.48202267 | 19.28398199 |
| ENSMUSG00000106705 | −2.05446715 | 2.41E−09 | 2.41E−07 | 18.94630097 | 32.05037489 | 31.22168513 |
| ENSMUSG00000106706 | −1.806072384 | 0.002749571 | 0.020715964 | 9.971737353 | 8.741011335 | 4.591424283 |
| ENSMUSG00000106943 | −1.230168101 | 0.009404129 | 0.049324655 | 9.971737353 | 13.59712874 | 13.77427285 |
| ENSMUSG00000107168 | −2.535260536 | 5.76E−05 | 0.00118059 | 1.994347471 | 9.712234816 | 5.50970914 |
| ENSMUSG00000107225 | 3.314781204 | 7.31E−09 | 6.61E−07 | 3.988694941 | 1.942446963 | 6.427993997 |
| ENSMUSG00000107304 | −1.772473058 | 0.001145745 | 0.010886341 | 7.977389882 | 10.6834583 | 11.93770314 |
| ENSMUSG00000107390 | −1.580957843 | 0.000625578 | 0.007061263 | 12.96325856 | 23.30936356 | 11.93770314 |
| ENSMUSG00000107624 | −2.865902972 | 2.57E−10 | 3.34E−08 | 8.974563618 | 13.59712874 | 11.93770314 |
| ENSMUSG00000108368 | −1.782724603 | 5.57E−05 | 0.001147878 | 20.94064844 | 11.65468178 | 7.346278854 |
| ENSMUSG00000108633 | −1.682864395 | 0.001365295 | 0.012400579 | 8.974563618 | 12.62590526 | 9.182848567 |
| ENSMUSG00000108820 | −1.905612833 | 0.000888445 | 0.00904159 | 6.980216147 | 3.884893927 | 8.26456371 |
| ENSMUSG00000108825 | 1.256782178 | 0.000111922 | 0.001987343 | 104.7032422 | 117.5180413 | 191.0032502 |
| ENSMUSG00000109089 | −1.779914445 | 1.06E−06 | 4.61E−05 | 42.87847062 | 34.96404534 | 96.41990995 |
| ENSMUSG00000109115 | 2.077699358 | 0.000502824 | 0.006083704 | 26.92369085 | 27.19425749 | 19.28398199 |
| ENSMUSG00000109157 | −1.885864825 | 0.000870552 | 0.00892677 | 4.985868677 | 6.798564371 | 8.26456371 |
| ENSMUSG00000109262 | 1.329001341 | 0.002840836 | 0.0212393 | 48.86151303 | 45.64750364 | 24.79369113 |
| ENSMUSG00000109291 | −1.949878064 | 0.001232984 | 0.01148511 | 1.994347471 | 9.712234816 | 9.182848567 |
| ENSMUSG00000109536 | −1.039899156 | 0.001827878 | 0.015463254 | 63.81911906 | 37.87771578 | 29.38511541 |
| ENSMUSG00000109555 | −1.255499819 | 0.007385438 | 0.041635066 | 10.96891109 | 14.56835222 | 15.61084256 |
| ENSMUSG00000109807 | −2.757236067 | 2.47E−09 | 2.44E−07 | 11.96608482 | 5.82734089 | 12.85598799 |
| ENSMUSG00000109836 | 1.535086391 | 0.000730915 | 0.007885666 | 46.86716556 | 68.9568672 | 89.0736311 |
| ENSMUSG00000109841 | 1.421032277 | 0.000627493 | 0.007061263 | 46.86716556 | 57.30218542 | 33.05825484 |
| ENSMUSG00000110588 | −5.501119532 | 2.73E−06 | 0.000102051 | 31.90955953 | 14.56835222 | 3.673139427 |
| ENSMUSG00000110613 | 1.168711997 | 0.002147567 | 0.017360028 | 64.81629279 | 58.2734089 | 38.56796398 |
| ENSMUSG00000110702 | −1.389252147 | 0.004080864 | 0.027194141 | 18.94630097 | 11.65468178 | 8.26456371 |
| ENSMUSG00000110755 | 1.852337964 | 4.42E−06 | 0.000147317 | 194.4488784 | 92.26623075 | 176.3106925 |
| ENSMUSG00000111282 | −1.321989153 | 0.002140143 | 0.017319005 | 21.93782218 | 18.45324615 | 30.30340027 |
| ENSMUSG00000111312 | 1.254957582 | 0.001977069 | 0.016387107 | 45.86999182 | 33.99282186 | 56.93366111 |
| ENSMUSG00000111631 | −1.948317608 | 1.41E−05 | 0.000376102 | 11.96608482 | 15.53957571 | 11.01941828 |
| ENSMUSG00000111709 | 2.018073354 | 0.00211263 | 0.017179449 | 31.90955953 | 69.92809068 | 7.346278854 |
| ENSMUSG00000111774 | 2.335615682 | 0.000501244 | 0.006070575 | 17.94912724 | 15.53957571 | 39.48624884 |

| ID | agedliverpbs1 | agedliverpbs2 | agedliverpbs3 | Gene.name |
|---|---|---|---|---|
| ENSMUSG00000000204 | 61.79909158 | 46.97322185 | 22.5130033 | Slfn4 |
| ENSMUSG00000000317 | 92.69863737 | 140.9196656 | 205.5535084 | Bcl6b |
| ENSMUSG00000000686 | 171.5457542 | 161.796653 | 294.6266953 | Abhd15 |
| ENSMUSG00000001227 | 171.5457542 | 203.550628 | 184.9981575 | Sema6b |
| ENSMUSG00000001403 | 51.14407579 | 37.57857748 | 17.61887214 | Ube2c |
| ENSMUSG00000001983 | 128.9256911 | 122.1303768 | 219.2570756 | Taco1 |
| ENSMUSG00000002233 | 330.3054895 | 288.1024274 | 298.5420002 | Rhoc |
| ENSMUSG00000002250 | 627.58043 | 657.6251059 | 647.9829644 | Ppard |
| ENSMUSG00000002289 | 1865.693265 | 2130.496573 | 2428.467877 | Angptl4 |
| ENSMUSG00000002831 | 228.0173379 | 574.117156 | 226.1088592 | Plin4 |
| ENSMUSG00000003032 | 155.5632305 | 107.5164856 | 114.5226689 | Klf4 |
| ENSMUSG00000003348 | 13.85152053 | 30.27163186 | 33.28009183 | Mob3a |
| ENSMUSG00000003500 | 174.742259 | 99.16569058 | 90.05201318 | Impdh1 |
| ENSMUSG00000003541 | 105.4846563 | 45.92937248 | 44.04718036 | Ier3 |
| ENSMUSG00000003848 | 522.0957737 | 291.2339755 | 260.3677772 | Nob1 |
| ENSMUSG00000004100 | 1249.833352 | 729.6507128 | 351.3986167 | Ppan |
| ENSMUSG00000004933 | 42.62006316 | 14.61389124 | 19.57652461 | Matk |
| ENSMUSG00000004951 | 237.6068521 | 636.7481184 | 278.9654756 | Hspb1 |
| ENSMUSG00000005148 | 19.17902842 | 27.14008374 | 22.5130033 | Klf5 |
| ENSMUSG00000005547 | 5271.036311 | 4402.956662 | 4447.78639 | Cyp2a5 |
| ENSMUSG00000005580 | 86.3056279 | 93.9464437 | 169.3369378 | Adcy9 |
| ENSMUSG00000006050 | 2542.286767 | 2021.936238 | 1365.462591 | Sra1 |
| ENSMUSG00000006134 | 225.8863347 | 252.6115486 | 375.8692724 | Crkl |
| ENSMUSG00000006517 | 1097.466626 | 579.3364028 | 613.7240464 | Mvd |
| ENSMUSG00000006587 | 10.65501579 | 2.087698749 | 4.894131151 | Snai3 |
| ENSMUSG00000006711 | 210.9693126 | 141.9635149 | 177.1675477 | D130043K22Rik |
| ENSMUSG00000006777 | 8.524012632 | 8.350794996 | 10.76708853 | Krt23 |
| ENSMUSG00000008153 | 96.96064369 | 120.0426781 | 78.30609842 | Clstn3 |
| ENSMUSG00000009013 | 329.2399879 | 396.6627623 | 281.9019543 | Dynll1 |
| ENSMUSG00000009633 | 2971.683904 | 3862.242686 | 1956.673634 | G0s2 |
| ENSMUSG00000014547 | 21.31003158 | 36.53472811 | 46.98365905 | Wdfy2 |
| ENSMUSG00000014609 | 14.91702211 | 14.61389124 | 15.66121968 | Chrne |
| ENSMUSG00000015224 | 318.5849721 | 255.7430968 | 158.5698493 | Cyp2j9 |
| ENSMUSG00000015312 | 121.46718 | 59.49941435 | 63.62370497 | Gadd45b |
| ENSMUSG00000016128 | 199.2487953 | 218.1645193 | 304.4149576 | Stard13 |
| ENSMUSG00000016356 | 90.56763421 | 172.2351468 | 124.3109312 | Col20a1 |
| ENSMUSG00000017737 | 38.35805684 | 32.35933061 | 22.5130033 | Mmp9 |
| ENSMUSG00000017868 | 543.4058053 | 586.6433485 | 498.2225512 | Sgk2 |
| ENSMUSG00000018486 | 3.196504737 | 15.65774062 | 6.851783612 | Wnt9b |
| ENSMUSG00000019082 | 7723.820946 | 7053.290223 | 5201.482588 | Slc25a22 |
| ENSMUSG00000019726 | 179.0042653 | 147.1827618 | 268.1983871 | Lyst |
| ENSMUSG00000019737 | 45.8165679 | 15.65774062 | 18.59769837 | Syne4 |
| ENSMUSG00000019883 | 200.3142968 | 230.6907118 | 405.2340593 | Echdc1 |
| ENSMUSG00000020018 | 268.5063979 | 250.5238499 | 170.3157641 | Snrpf |
| ENSMUSG00000020027 | 2138.461669 | 846.5618427 | 589.2533906 | Socs2 |
| ENSMUSG00000020091 | 393.1700826 | 471.8199173 | 1129.56547 | Eif4ebp2 |
| ENSMUSG00000020122 | 2670.146957 | 1807.947117 | 1885.219319 | Egfr |
| ENSMUSG00000020335 | 24.50653632 | 41.75397498 | 32.3012656 | Zfp354b |
| ENSMUSG00000020429 | 2643.509417 | 2014.629293 | 3936.839098 | Igfbp1 |
| ENSMUSG00000020441 | 303.66795 | 273.4885361 | 168.3581116 | 2310033P09Rik |
| ENSMUSG00000020532 | 2810.793165 | 1739.053058 | 2484.260972 | Acaca |
| ENSMUSG00000020641 | 408.0871047 | 425.8905448 | 452.2177184 | Rsad2 |
| ENSMUSG00000020656 | 71.38860579 | 76.20100434 | 73.41196727 | Grhl1 |
| ENSMUSG00000020681 | 14.91702211 | 21.92083686 | 12.72474099 | Ace |
| ENSMUSG00000020692 | 249.3273695 | 152.4020087 | 104.7344066 | Nle1 |
| ENSMUSG00000020812 | 180.0697668 | 73.06945621 | 57.75074759 | 1810032O08Rik |
| ENSMUSG00000020889 | 443.2486569 | 371.6103773 | 433.62002 | Nr1d1 |
| ENSMUSG00000020917 | 9135.610538 | 6014.660096 | 10059.39717 | Acly |
| ENSMUSG00000020948 | 9.589514211 | 20.87698749 | 44.04718036 | Klhl28 |
| ENSMUSG00000020961 | 25.5720379 | 35.49087873 | 64.6025312 | Ston2 |
| ENSMUSG00000021250 | 204.5763032 | 115.8672806 | 135.0780198 | Fos |
| ENSMUSG00000021260 | 6.393009474 | 11.48234312 | 10.76708853 | Hhipl1 |
| ENSMUSG00000021416 | 404.8906 | 324.6371555 | 254.4948199 | Eci3 |
| ENSMUSG00000021453 | 979.1959511 | 568.8979091 | 678.3265776 | Gadd45g |
| ENSMUSG00000021611 | 220.5588268 | 227.5591636 | 197.7228985 | Tert |
| ENSMUSG00000021670 | 3051.596522 | 1273.496237 | 2047.704474 | Hmgcr |
| ENSMUSG00000021684 | 54.34058053 | 72.02560684 | 49.92013774 | Pde8b |
| ENSMUSG00000021773 | 44.75106632 | 55.32401685 | 29.36478691 | Comtd1 |
| ENSMUSG00000021775 | 635.0389411 | 274.5323855 | 351.3986167 | Nr1d2 |
| ENSMUSG00000021804 | 7.458511053 | 11.48234312 | 20.55535084 | Rgr |
| ENSMUSG00000021958 | 56.47158369 | 91.85874496 | 47.96248528 | Pinx1 |
| ENSMUSG00000022383 | 440.0521521 | 565.766361 | 794.806899 | Ppara |
| ENSMUSG00000022389 | 599.877389 | 502.0915491 | 601.9781316 | Tef |
| ENSMUSG00000022408 | 38.35805684 | 32.35933061 | 21.53417707 | Fam83f |
| ENSMUSG00000022528 | 643.5629537 | 588.7310472 | 500.1802037 | Hes1 |
| ENSMUSG00000022651 | 52.20957737 | 37.57857748 | 32.3012656 | Retnlg |
| ENSMUSG00000022704 | 291.9474326 | 188.9367368 | 134.0991935 | Qtrt2 |
| ENSMUSG00000022853 | 4279.054341 | 3944.706786 | 4891.194673 | Ehhadh |
| ENSMUSG00000022883 | 166.2182463 | 116.9111299 | 103.7555804 | Robo1 |
| ENSMUSG00000022887 | 350.5500195 | 374.7419254 | 819.2775547 | Masp1 |
| ENSMUSG00000022911 | 105.4846563 | 73.06945621 | 75.36961973 | Arl13b |
| ENSMUSG00000023034 | 572.1743479 | 134.6565693 | 149.7604132 | Nr4a1 |
| ENSMUSG00000023044 | 4109.63959 | 5560.585618 | 7290.297763 | Csad |
| ENSMUSG00000023052 | 36.22705368 | 52.19246872 | 20.55535084 | Npff |
| ENSMUSG00000023067 | 1114.514652 | 1009.402345 | 858.4306039 | Cdkn1a |
| ENSMUSG00000023073 | 369.7290479 | 140.9196656 | 157.5910231 | Slc10a2 |
| ENSMUSG00000023341 | 264.2443916 | 178.498243 | 164.4428067 | Mx2 |
| ENSMUSG00000023571 | 53.27507895 | 58.45556497 | 25.44948199 | C1qtnf12 |
| ENSMUSG00000023800 | 160.8907384 | 217.1206699 | 156.6121968 | Tiam2 |
| ENSMUSG00000023905 | 328.1744863 | 207.7260255 | 109.6285378 | Tnfrsf12a |
| ENSMUSG00000023927 | 61.79909158 | 60.54326372 | 33.28009183 | Satb1 |
| ENSMUSG00000023968 | 20.24453 | 38.62242686 | 33.28009183 | Crip3 |
| ENSMUSG00000024118 | 1463.99917 | 1896.674313 | 1329.246021 | Tedc2 |
| ENSMUSG00000024130 | 1006.898992 | 1228.610714 | 2261.088592 | Abca3 |
| ENSMUSG00000024136 | 64.99559632 | 52.19246872 | 34.25891806 | Dnase1l2 |
| ENSMUSG00000024190 | 1247.702349 | 1464.520672 | 1004.275712 | Dusp1 |
| ENSMUSG00000024236 | 484.8032184 | 345.514143 | 575.5498234 | Svil |
| ENSMUSG00000024411 | 14.91702211 | 13.57004187 | 17.61887214 | Aqp4 |
| ENSMUSG00000024440 | 35.16155211 | 70.98175747 | 72.43314104 | Pcdh12 |
| ENSMUSG00000024665 | 3625.901873 | 3398.773563 | 5157.435407 | Fads2 |
| ENSMUSG00000024843 | 3171.998201 | 2942.611387 | 2249.342677 | Chka |
| ENSMUSG00000024887 | 191.7902842 | 179.5420924 | 296.5843478 | Asah2 |
| ENSMUSG00000024924 | 269.5718995 | 262.006193 | 320.0761773 | Vldlr |
| ENSMUSG00000024970 | 90.56763421 | 63.67481184 | 65.58135743 | Al846148 |
| ENSMUSG00000024978 | 1324.418463 | 791.2378259 | 2079.026913 | Gpam |
| ENSMUSG00000025003 | 152.3667258 | 144.0512137 | 186.95581 | Cyp2c39 |
| ENSMUSG00000025006 | 107.6156595 | 110.6480337 | 207.5111608 | Sorbs1 |
| ENSMUSG00000025153 | 35454.56504 | 23677.63536 | 26074.95195 | Fasn |
| ENSMUSG00000025161 | 61.79909158 | 26.09623436 | 32.3012656 | Slc16a3 |
| ENSMUSG00000025240 | 183.2662716 | 163.8843518 | 418.9376266 | Sacm1l |
| ENSMUSG00000025323 | 71.38860579 | 52.19246872 | 60.68722628 | Sp4 |
| ENSMUSG00000025402 | 646.7594584 | 568.8979091 | 502.1378561 | Nab2 |
| ENSMUSG00000025429 | 188.5937795 | 118.9988287 | 253.5159936 | Pstpip2 |
| ENSMUSG00000025450 | 145.9737163 | 103.3410881 | 105.7132329 | Gm9752 |
| ENSMUSG00000025997 | 39.42355842 | 58.45556497 | 22.5130033 | Ikzf2 |
| ENSMUSG00000026020 | 828.9602284 | 520.8808379 | 466.9001118 | Nop58 |
| ENSMUSG00000026249 | 45.8165679 | 122.1303768 | 117.4591476 | Serpine2 |
| ENSMUSG00000026358 | 104.4191547 | 58.45556497 | 96.9037968 | Rgs1 |
| ENSMUSG00000026398 | 615.8599126 | 631.5288716 | 846.6846892 | Nr5a2 |
| ENSMUSG00000026471 | 181.1352684 | 104.3849374 | 126.2685837 | Mr1 |
| ENSMUSG00000026475 | 1039.929541 | 2826.744106 | 2390.293654 | Rgs16 |
| ENSMUSG00000026525 | 43.68556474 | 19.83313812 | 16.64004591 | Opn3 |
| ENSMUSG00000026822 | 304.7334516 | 223.3837661 | 189.8922887 | Lcn2 |
| ENSMUSG00000026826 | 69.25760263 | 25.05238499 | 13.70356722 | Nr4a2 |
| ENSMUSG00000026832 | 101.22265 | 42.79782435 | 62.64487874 | Cytip |
| ENSMUSG00000027360 | 370.7945495 | 319.4179086 | 181.0828526 | Hdc |
| ENSMUSG00000027398 | 98.02614527 | 82.46410059 | 128.2262362 | Il1b |
| ENSMUSG00000027405 | 920.5933642 | 450.9429298 | 352.3774429 | Nop56 |
| ENSMUSG00000027496 | 55.40608211 | 66.80635997 | 66.56018366 | Aurka |
| ENSMUSG00000027513 | 48173.45739 | 29794.5927 | 39823.54518 | Pck1 |
| ENSMUSG00000027605 | 8380.169919 | 3799.611723 | 6353.561061 | Acss2 |
| ENSMUSG00000027762 | 637.1699442 | 555.3278672 | 690.0724923 | Sucnr1 |
| ENSMUSG00000027907 | 207.7728079 | 117.9549793 | 139.9721509 | S100a11 |
| ENSMUSG00000027947 | 713.8860579 | 763.0538928 | 1358.610808 | Il6ra |
| ENSMUSG00000028008 | 20.24453 | 24.00853561 | 36.21657052 | Asic5 |
| ENSMUSG00000028339 | 295.1439374 | 274.5323855 | 289.7325642 | Col15a1 |
| ENSMUSG00000028445 | 91.63313579 | 280.7954817 | 425.7894102 | Enho |
| ENSMUSG00000028630 | 124.6636847 | 101.2533893 | 226.1088592 | Dyrk2 |
| ENSMUSG00000028838 | 74.58511053 | 49.0609206 | 78.30609842 | Extl1 |
| ENSMUSG00000028859 | 152.3667258 | 96.03414245 | 74.3907935 | Csf3r |
| ENSMUSG00000028862 | 77.78161526 | 40.71012561 | 57.75074759 | Map3k6 |
| ENSMUSG00000028864 | 39.42355842 | 69.93790809 | 111.5861902 | Hgf |
| ENSMUSG00000028957 | 62.86459316 | 53.2363181 | 46.00483282 | Per3 |
| ENSMUSG00000028976 | 42.62006316 | 34.44702936 | 45.02600659 | Slc2a5 |
| ENSMUSG00000029086 | 210.9693126 | 165.9720505 | 243.7277313 | Prom1 |
| ENSMUSG00000029135 | 499.7202405 | 272.4446867 | 294.6266953 | Fosl2 |
| ENSMUSG00000029188 | 186.4627763 | 179.5420924 | 182.0616788 | Slc34a2 |
| ENSMUSG00000029195 | 332.4364926 | 209.8137243 | 422.8529315 | Klb |
| ENSMUSG00000029370 | 190.7247826 | 137.7881174 | 169.3369378 | Rassf6 |
| ENSMUSG00000029373 | 25.5720379 | 28.18393311 | 18.59769837 | Pf4 |
| ENSMUSG00000029380 | 615.8599126 | 454.0744779 | 700.8395809 | Cxcl1 |
| ENSMUSG00000029580 | 16369.30076 | 20515.81561 | 13266.0319 | Actb |
| ENSMUSG00000029591 | 177.9387637 | 131.5250212 | 112.5650165 | Ung |
| ENSMUSG00000029656 | 429.3971363 | 614.8272816 | 554.0156463 | C8b |
| ENSMUSG00000030032 | 43.68556474 | 55.32401685 | 35.23774429 | Wdr54 |
| ENSMUSG00000030055 | 75.65061211 | 74.11330559 | 171.2945903 | Rab43 |
| ENSMUSG00000030691 | 189.6592811 | 180.5859418 | 227.0876854 | Fchsd2 |
| ENSMUSG00000030782 | 115.0741705 | 67.85020934 | 70.47548858 | Tgfb1i1 |
| ENSMUSG00000030814 | 814.0432063 | 869.526529 | 459.069502 | Bcl7c |
| ENSMUSG00000030827 | 120.4016784 | 80.37640184 | 48.94131151 | Fgf21 |
| ENSMUSG00000030934 | 5865.586192 | 5683.759844 | 6052.082582 | Oat |
| ENSMUSG00000030968 | 131.0566942 | 132.5688706 | 93.9673181 | Pdilt |
| ENSMUSG00000031010 | 414.4801142 | 361.1718836 | 775.2303744 | Usp9x |
| ENSMUSG00000031271 | 1418.182602 | 1115.874981 | 1330.224847 | Serpina7 |
| ENSMUSG00000031378 | 429.3971363 | 392.4873648 | 441.4506298 | Abcd1 |
| ENSMUSG00000031465 | 50.07857421 | 30.27163186 | 47.96248528 | Angpt2 |
| ENSMUSG00000031762 | 9477.636545 | 4672.2698 | 1607.23267 | Mt2 |
| ENSMUSG00000031765 | 20312.7221 | 10174.39985 | 4243.211708 | Mt1 |
| ENSMUSG00000032009 | 377.187559 | 338.2071973 | 562.8250824 | Sesn3 |
| ENSMUSG00000032064 | 268.5063979 | 302.7163186 | 381.7422298 | Dixdc1 |
| ENSMUSG00000032083 | 163165.5843 | 112907.9676 | 104422.1611 | Apoa1 |
| ENSMUSG00000032091 | 20.24453 | 8.350794996 | 4.894131151 | Tmprss4 |
| ENSMUSG00000032285 | 26.63753947 | 15.65774062 | 44.04718036 | Dnaja4 |
| ENSMUSG00000032417 | 26.63753947 | 43.84167373 | 17.61887214 | Rwdd2a |
| ENSMUSG00000032418 | 6053.11447 | 3739.068459 | 4136.519649 | Me1 |
| ENSMUSG00000032500 | 75.65061211 | 69.93790809 | 123.332105 | Dclk3 |
| ENSMUSG00000032561 | 453.9036726 | 141.9635149 | 317.1396986 | Acpp |
| ENSMUSG00000032702 | 468.8206947 | 629.4411728 | 864.3035613 | Kank1 |
| ENSMUSG00000032724 | 234.4103474 | 176.4105443 | 323.9914822 | Abtb2 |
| ENSMUSG00000032735 | 436.8556474 | 741.1330559 | 606.8722628 | Ablim3 |
| ENSMUSG00000032786 | 7150.581096 | 4035.521682 | 3301.580875 | Alas1 |
| ENSMUSG00000032849 | 168.3492495 | 143.0073643 | 195.7652461 | Abcc4 |
| ENSMUSG00000032860 | 155.5632305 | 134.6565693 | 141.9298034 | P2ry2 |
| ENSMUSG00000032883 | 856.6632695 | 580.3802522 | 723.3525842 | Acsl3 |
| ENSMUSG00000033105 | 1574.811334 | 996.8761526 | 1567.100795 | Lss |
| ENSMUSG00000033594 | 852.4012632 | 820.4656084 | 539.3332529 | Spata2l |
| ENSMUSG00000033624 | 105.4846563 | 98.1218412 | 178.1463739 | Pdpr |
| ENSMUSG00000033792 | 21.31003158 | 26.09623436 | 43.06835413 | Atp7a |
| ENSMUSG00000033855 | 37.29255526 | 42.79782435 | 77.32727219 | Ston1 |
| ENSMUSG00000033967 | 15.98252368 | 19.83313812 | 39.15304921 | Rnf225 |
| ENSMUSG00000034066 | 96.96064369 | 107.5164856 | 184.9981575 | Farp2 |
| ENSMUSG00000034110 | 55.40608211 | 37.57857748 | 55.79309512 | Kctd7 |
| ENSMUSG00000034271 | 135.3187005 | 37.57857748 | 64.6025312 | Jdp2 |
| ENSMUSG00000034755 | 19.17902842 | 36.53472811 | 20.55535084 | Pcdh11x |
| ENSMUSG00000034765 | 166.2182463 | 53.2363181 | 31.32243937 | Dusp5 |
| ENSMUSG00000034853 | 165.1527447 | 137.7881174 | 147.8027608 | Acot11 |
| ENSMUSG00000034926 | 6118.110066 | 6691.074491 | 12774.66113 | Dhcr24 |
| ENSMUSG00000035078 | 94.82964053 | 101.2533893 | 258.4101248 | Mtmr9 |
| ENSMUSG00000035112 | 43.68556474 | 45.92937248 | 36.21657052 | Wnk4 |
| ENSMUSG00000035164 | 61.79909158 | 70.98175747 | 77.32727219 | Zc3h12c |
| ENSMUSG00000035165 | 144.9082147 | 55.32401685 | 91.03083941 | Kcne3 |
| ENSMUSG00000035284 | 293.0129342 | 218.1645193 | 482.5613315 | Vps13c |
| ENSMUSG00000035900 | 147.0392179 | 160.7528037 | 191.8499411 | Gramd4 |
| ENSMUSG00000035933 | 77.78161526 | 81.42025121 | 127.2474099 | Cog5 |
| ENSMUSG00000035948 | 193.9212874 | 154.4897074 | 418.9376266 | Acss3 |
| ENSMUSG00000036062 | 27.70304105 | 29.22778249 | 12.72474099 | Phf24 |
| ENSMUSG00000036120 | 773.5541463 | 626.3096247 | 392.5093183 | Rfxank |
| ENSMUSG00000036611 | 415.5456158 | 267.2254399 | 309.3090888 | Eepd1 |
| ENSMUSG00000037035 | 141.71171 | 149.2704606 | 192.8287674 | Inhbb |
| ENSMUSG00000037071 | 157237.1335 | 63623.66322 | 106805.6029 | Scd1 |
| ENSMUSG00000037095 | 5413.813523 | 8132.630477 | 6142.134595 | Lrg1 |
| ENSMUSG00000037157 | 11.72051737 | 14.61389124 | 24.47065576 | Il22ra1 |
| ENSMUSG00000037336 | 23.44103474 | 42.79782435 | 31.32243937 | Mfsd2b |
| ENSMUSG00000037443 | 3091.020081 | 2288.117829 | 2056.51391 | Cep85 |
| ENSMUSG00000037447 | 99.09164684 | 54.28016747 | 54.81426889 | Arid5a |
| ENSMUSG00000037465 | 592.4188779 | 817.3340602 | 760.5479809 | Klf10 |
| ENSMUSG00000037583 | 304.7334516 | 631.5288716 | 569.676866 | Nr0b2 |
| ENSMUSG00000037709 | 239.7378553 | 289.1462767 | 357.271574 | Fam13a |
| ENSMUSG00000037887 | 80.97812 | 20.87698749 | 30.34361314 | Dusp8 |
| ENSMUSG00000038217 | 1346.793996 | 1375.793476 | 1970.377202 | Tlcd2 |
| ENSMUSG00000038233 | 284.4889216 | 256.7869461 | 354.3350954 | Fam198a |
| ENSMUSG00000038253 | 112.9431674 | 83.50794996 | 44.04718036 | Hoxa5 |
| ENSMUSG00000038370 | 277.0304105 | 627.3534741 | 676.3689251 | Pcp4l1 |
| ENSMUSG00000038415 | 331.3709911 | 448.855231 | 695.9454497 | Foxq1 |
| ENSMUSG00000038418 | 515.7027642 | 342.3825948 | 912.2660466 | Egr1 |
| ENSMUSG00000038473 | 45.8165679 | 35.49087873 | 46.98365905 | Nos1ap |
| ENSMUSG00000038530 | 64.99559632 | 30.27163186 | 63.62370497 | Rgs4 |
| ENSMUSG00000038583 | 67.12659947 | 41.75397498 | 16.64004591 | Pln |
| ENSMUSG00000038587 | 349.4845179 | 176.4105443 | 253.5159936 | Akap12 |
| ENSMUSG00000038751 | 46.88206947 | 44.8855231 | 44.04718036 | Ptk6 |
| ENSMUSG00000038768 | 106.5501579 | 77.24485371 | 93.9673181 | 9130409I23Rik |
| ENSMUSG00000038774 | 194.986789 | 189.9805862 | 328.8856134 | Ascc3 |
| ENSMUSG00000038844 | 169.4147511 | 146.1389124 | 248.6218625 | Kif16b |
| ENSMUSG00000038895 | 230.1483411 | 226.5153143 | 190.8711149 | Zfp653 |
| ENSMUSG00000039103 | 68.19210105 | 74.11330559 | 43.06835413 | Nexn |
| ENSMUSG00000039304 | 105.4846563 | 73.06945621 | 81.24257711 | Tnfsf10 |
| ENSMUSG00000039533 | 167.2837479 | 54.28016747 | 122.3532788 | Mmd2 |
| ENSMUSG00000039601 | 337.7640005 | 298.5409211 | 280.9231281 | Rcan2 |
| ENSMUSG00000039704 | 59.66808842 | 50.10476998 | 182.0616788 | Lmbrd2 |
| ENSMUSG00000039741 | 105.4846563 | 146.1389124 | 137.0356722 | Bahcc1 |
| ENSMUSG00000039853 | 126.7946879 | 156.5774062 | 258.4101248 | Trim14 |
| ENSMUSG00000039981 | 53.27507895 | 77.24485371 | 62.64487874 | Zc3h12d |
| ENSMUSG00000040093 | 75.65061211 | 69.93790809 | 132.1415411 | Bmf |
| ENSMUSG00000040128 | 3368.050491 | 2671.210549 | 2388.336002 | Pnrc1 |
| ENSMUSG00000040152 | 91.63313579 | 40.71012561 | 85.15788203 | Thbs1 |
| ENSMUSG00000040435 | 430.4626379 | 241.1292055 | 201.6382034 | Ppp1r15a |
| ENSMUSG00000040584 | 118.2706753 | 124.2180756 | 233.939469 | Abcb1a |
| ENSMUSG00000040855 | 259.9823853 | 223.3837661 | 409.1493642 | Reps2 |
| ENSMUSG00000040891 | 1650.461946 | 1922.770548 | 1525.990093 | Foxa3 |
| ENSMUSG00000041134 | 18.11352684 | 31.31548123 | 39.15304921 | Cyyr1 |
| ENSMUSG00000041372 | 9.589514211 | 12.52619249 | 9.788262303 | B4galnt3 |
| ENSMUSG00000041695 | 101.22265 | 78.28870309 | 41.11070167 | Kcnj2 |
| ENSMUSG00000041702 | 59.66808842 | 58.45556497 | 97.88262303 | Btbd7 |
| ENSMUSG00000041920 | 586.0258684 | 355.9526367 | 312.2455675 | Slc16a6 |
| ENSMUSG00000041930 | 157.6942337 | 126.3057743 | 71.45431481 | Fam222a |
| ENSMUSG00000041945 | 3.196504737 | 5.219246872 | 10.76708853 | Mfsd9 |
| ENSMUSG00000042010 | 4018.006454 | 2149.285862 | 2550.821156 | Acacb |
| ENSMUSG00000042115 | 117.2051737 | 124.2180756 | 131.1627149 | Klhdc8a |
| ENSMUSG00000042246 | 77.78161526 | 33.40317998 | 54.81426889 | Tmc7 |
| ENSMUSG00000042333 | 30.89954579 | 27.14008374 | 41.11070167 | Tnfrsf14 |
| ENSMUSG00000042354 | 922.7243674 | 424.8466954 | 389.5728396 | Gnl3 |
| ENSMUSG00000042379 | 140.6462084 | 79.33255246 | 203.5958559 | Esm1 |
| ENSMUSG00000042444 | 255.720379 | 221.2960674 | 319.0973511 | Mindy2 |
| ENSMUSG00000042510 | 94.82964053 | 45.92937248 | 47.96248528 | AA986860 |
| ENSMUSG00000042607 | 37.29255526 | 24.00853561 | 42.0895279 | Asb4 |
| ENSMUSG00000042622 | 89.50213263 | 50.10476998 | 72.43314104 | Maff |
| ENSMUSG00000042680 | 87.37112948 | 114.8234312 | 243.7277313 | Garem1 |
| ENSMUSG00000042743 | 15.98252368 | 14.61389124 | 26.42830822 | Sgtb |
| ENSMUSG00000042745 | 726.6720769 | 889.3596671 | 654.834748 | Id1 |
| ENSMUSG00000043165 | 13.85152053 | 41.75397498 | 38.17422298 | Lor |
| ENSMUSG00000043421 | 138.5152053 | 55.32401685 | 75.36961973 | Hilpda |
| ENSMUSG00000043639 | 20.24453 | 46.97322185 | 49.92013774 | Rbm20 |
| ENSMUSG00000043681 | 543.4058053 | 474.9514654 | 382.721056 | Fam25c |
| ENSMUSG00000044042 | 108.6811611 | 85.59564871 | 203.5958559 | Fmn1 |
| ENSMUSG00000044186 | 17.04802526 | 10.43849374 | 17.61887214 | Nkx2-6 |
| ENSMUSG00000044339 | 61.79909158 | 89.77104621 | 79.28492465 | Alkbh2 |
| ENSMUSG00000044349 | 148.1047195 | 83.50794996 | 50.89896397 | Snhg11 |
| ENSMUSG00000044359 | 69.25760263 | 76.20100434 | 148.781587 | P2ry4 |
| ENSMUSG00000044676 | 609.4669032 | 360.1280342 | 270.1560396 | Zfp612 |
| ENSMUSG00000044749 | 1050.584557 | 874.7457758 | 2096.645785 | Abca6 |
| ENSMUSG00000044948 | 73.51960895 | 52.19246872 | 59.70840005 | Cfap43 |
| ENSMUSG00000045045 | 38.35805684 | 38.62242686 | 56.77192136 | Lrfn4 |
| ENSMUSG00000045294 | 8335.418852 | 7066.860265 | 6456.337815 | Insig1 |
| ENSMUSG00000045348 | 38.35805684 | 44.8855231 | 20.55535084 | Nyap1 |
| ENSMUSG00000045382 | 89.50213263 | 68.89405872 | 29.36478691 | Cxcr4 |
| ENSMUSG00000045411 | 798.0606826 | 403.9697079 | 303.4361314 | 2410002F23Rik |
| ENSMUSG00000045776 | 514.6372626 | 321.5056073 | 424.8105839 | Lrtm1 |
| ENSMUSG00000045875 | 28.76854263 | 45.92937248 | 72.43314104 | Adra1a |
| ENSMUSG00000046541 | 22.37553316 | 22.96468624 | 41.11070167 | Zfp526 |
| ENSMUSG00000046721 | 46.88206947 | 46.97322185 | 42.0895279 | Rpl14-ps1 |
| ENSMUSG00000046908 | 36.22705368 | 28.18393311 | 24.47065576 | Ltb4r1 |
| ENSMUSG00000047496 | 124.6636847 | 234.8661093 | 298.5420002 | Rnf152 |
| ENSMUSG00000047649 | 210.9693126 | 155.5335568 | 111.5861902 | Cd3eap |
| ENSMUSG00000047875 | 49.01307263 | 46.97322185 | 47.96248528 | Gpr157 |
| ENSMUSG00000048191 | 53.27507895 | 40.71012561 | 27.40713445 | Muc6 |
| ENSMUSG00000048644 | 40.48906 | 37.57857748 | 23.49182953 | Ctxn1 |
| ENSMUSG00000048856 | 8878.824658 | 12487.57007 | 11318.1677 | Slc25a47 |
| ENSMUSG00000049044 | 3411.736056 | 3040.733228 | 2890.473858 | Rapgef4 |
| ENSMUSG00000049313 | 152.3667258 | 109.6041843 | 170.3157641 | Sorl1 |
| ENSMUSG00000049580 | 1707.999031 | 1943.647535 | 1562.206663 | Tsku |
| ENSMUSG00000049791 | 215.231319 | 290.1901261 | 450.2600659 | Fzd4 |
| ENSMUSG00000049950 | 94.82964053 | 44.8855231 | 35.23774429 | Rpp38 |
| ENSMUSG00000050390 | 692.5760263 | 634.6604197 | 674.4112726 | C77080 |
| ENSMUSG00000050503 | 25.5720379 | 38.62242686 | 16.64004591 | Fbxl22 |
| ENSMUSG00000050663 | 15.98252368 | 13.57004187 | 15.66121968 | Trhde |
| ENSMUSG00000050737 | 119.3361768 | 55.32401685 | 31.32243937 | Ptges |
| ENSMUSG00000050914 | 273.8339058 | 58.45556497 | 46.98365905 | Ankrd37 |
| ENSMUSG00000051149 | 6.393009474 | 3.131548123 | 21.53417707 | Adnp |
| ENSMUSG00000051339 | 917.3968595 | 914.4120521 | 942.6096597 | 2900026A02Rik |
| ENSMUSG00000051452 | 92.69863737 | 80.37640184 | 127.2474099 | Gm11437 |
| ENSMUSG00000051674 | 295.1439374 | 270.356988 | 333.7797445 | Dcun1d4 |
| ENSMUSG00000051998 | 42.62006316 | 39.66627623 | 21.53417707 | Lax1 |
| ENSMUSG00000052085 | 197.1177921 | 161.796653 | 227.0876854 | Dock8 |
| ENSMUSG00000052595 | 821.5017174 | 661.8005034 | 1485.858218 | A1cf |
| ENSMUSG00000052656 | 1950.933391 | 1650.325861 | 1785.379044 | Rnf103 |
| ENSMUSG00000052684 | 547.6678116 | 268.2692892 | 399.3611019 | Jun |
| ENSMUSG00000052713 | 23.44103474 | 18.78928874 | 42.0895279 | Zfp608 |
| ENSMUSG00000052837 | 1233.850828 | 697.2913822 | 1027.767542 | Junb |
| ENSMUSG00000053560 | 725.6065753 | 569.9417585 | 550.1003414 | Ier2 |
| ENSMUSG00000053964 | 348.4190163 | 621.0903778 | 388.5940134 | Lgals4 |
| ENSMUSG00000053977 | 57.53708526 | 21.92083686 | 40.13187544 | Cd8a |
| ENSMUSG00000054008 | 753.3096163 | 842.3864452 | 2156.354185 | Ndst1 |
| ENSMUSG00000054150 | 33.03054895 | 29.22778249 | 32.3012656 | Syne3 |
| ENSMUSG00000054422 | 57173.74923 | 60050.56682 | 33819.42508 | Fabp1 |
| ENSMUSG00000054453 | 69.25760263 | 36.53472811 | 39.15304921 | Sytl5 |
| ENSMUSG00000054659 | 15.98252368 | 25.05238499 | 65.58135743 | Pm20d2 |
| ENSMUSG00000054932 | 72.45410737 | 72.02560684 | 80.26375088 | Afp |
| ENSMUSG00000055148 | 476.2792058 | 390.3996661 | 293.6478691 | Klf2 |
| ENSMUSG00000055254 | 409.1526063 | 418.5835992 | 554.9944726 | Ntrk2 |
| ENSMUSG00000055491 | 706.4275469 | 442.5921348 | 309.3090888 | Pprc1 |
| ENSMUSG00000055660 | 29.83404421 | 36.53472811 | 60.68722628 | Mettl4 |
| ENSMUSG00000055692 | 90.56763421 | 57.4117156 | 44.04718036 | Tmem191c |
| ENSMUSG00000055980 | 114.0086689 | 159.7089543 | 270.1560396 | Irs1 |
| ENSMUSG00000056054 | 100.1571484 | 98.1218412 | 95.92497057 | S100a8 |
| ENSMUSG00000056071 | 140.6462084 | 203.550628 | 123.332105 | S100a9 |
| ENSMUSG00000056091 | 3671.718441 | 3506.290049 | 4394.929774 | St3gal5 |
| ENSMUSG00000056148 | 23.44103474 | 25.05238499 | 50.89896397 | Rdh9 |
| ENSMUSG00000056313 | 1762.339612 | 990.6130564 | 874.0918236 | Tcim |
| ENSMUSG00000057342 | 564.7158369 | 644.0550641 | 614.7028726 | Sphk2 |
| ENSMUSG00000057604 | 25.5720379 | 28.18393311 | 27.40713445 | Lmcd1 |
| ENSMUSG00000057722 | 323.91248 | 199.3752305 | 166.4004591 | Lepr |
| ENSMUSG00000057969 | 49.01307263 | 42.79782435 | 37.19539675 | Sema3b |
| ENSMUSG00000058207 | 48372.70618 | 79528.79614 | 62218.1105 | Serpina3k |
| ENSMUSG00000058503 | 376.1220574 | 383.0927204 | 227.0876854 | Fam133b |
| ENSMUSG00000058793 | 1397.938072 | 1320.469459 | 1959.610113 | Cds2 |
| ENSMUSG00000058794 | 71.38860579 | 26.09623436 | 30.34361314 | Nfe2 |
| ENSMUSG00000058921 | 261.0478868 | 329.8564023 | 683.2207087 | Slc10a5 |
| ENSMUSG00000059149 | 147.0392179 | 80.37640184 | 237.854774 | Mfsd4a |
| ENSMUSG00000059824 | 24.50653632 | 16.70158999 | 12.72474099 | Dbp |
| ENSMUSG00000060429 | 201.3797984 | 180.5859418 | 380.7634036 | Sntb1 |
| ENSMUSG00000061175 | 495.4582342 | 405.0135573 | 478.6460266 | Fnip2 |
| ENSMUSG00000061292 | 523.1612753 | 399.7943104 | 986.6568401 | Cyp3a59 |
| ENSMUSG00000061436 | 228.0173379 | 186.849038 | 275.0501707 | Hipk2 |
| ENSMUSG00000061536 | 88.43663105 | 94.99029308 | 144.866282i | Sec22c |
| ENSMUSG00000061825 | 298.3404421 | 160.7528037 | 130.1838886 | Ces2c |
| ENSMUSG00000062901 | 297.2749405 | 259.9184942 | 513.8837709 | Klhl24 |
| ENSMUSG00000063535 | 27.70304105 | 29.22778249 | 32.3012656 | Zfp773 |
| ENSMUSG00000063704 | 581.7638621 | 784.9747296 | 446.344761 | Mapk15 |
| ENSMUSG00000063929 | 208.8383095 | 167.0158999 | 174.231069 | Cyp4a32 |
| ENSMUSG00000065126 | 154.497729 | 158.6651049 | 77.32727219 | Snord104 |
| ENSMUSG00000065147 | 54.34058053 | 32.35933061 | 19.57652461 | Snora31 |
| ENSMUSG00000065952 | 51.14407579 | 55.32401685 | 76.34844596 | C330021F23Rik |
| ENSMUSG00000066456 | 70.32310421 | 34.44702936 | 38.17422298 | Hmgn3 |
| ENSMUSG00000066477 | 93.76413895 | 118.9988287 | 119.416800i | Gm16551 |
| ENSMUSG00000066687 | 472.0171995 | 943.6398345 | 1137.39608 | Zbtb16 |
| ENSMUSG00000066944 | 10.65501579 | 10.43849374 | 24.47065576 | NA |
| ENSMUSG00000067149 | 437.921149 | 878.9211733 | 284.838433 | Jchain |
| ENSMUSG00000068463 | 46.88206947 | 15.65774062 | 12.72474099 | B630019A10Rik |
| ENSMUSG00000068742 | 312.1919626 | 260.9623436 | 334.7585707 | Cry2 |
| ENSMUSG00000068877 | 201.3797984 | 256.7869461 | 255.473646i | Selenbp2 |
| ENSMUSG00000069456 | 2190.671246 | 1455.126028 | 1756.014257 | Rdh16 |
| ENSMUSG00000069804 | 42.62006316 | 55.32401685 | 34.25891806 | Gm10277 |
| ENSMUSG00000070576 | 98.02614527 | 132.5688706 | 122.3532788 | Mn1 |
| ENSMUSG00000070583 | 13.85152053 | 11.48234312 | 31.32243937 | Fv1 |
| ENSMUSG00000071076 | 3733.517533 | 2948.874483 | 1847.045097 | Jund |
| ENSMUSG00000071456 | 12.78601895 | 16.70158999 | 35.23774429 | 1110002L01Rik |
| ENSMUSG00000071547 | 90.56763421 | 104.3849374 | 70.47548858 | Nt5dc2 |
| ENSMUSG00000071637 | 239.7378553 | 123.1742262 | 171.2945903 | Cebpd |
| ENSMUSG00000071645 | 605.2048969 | 525.0562354 | 343.5680068 | Tut1 |
| ENSMUSG00000072294 | 24.50653632 | 18.78928874 | 60.68722628 | Klf12 |
| ENSMUSG00000072571 | 20.24453 | 29.22778249 | 29.36478691 | Tmem253 |
| ENSMUSG00000072664 | 273.8339058 | 440.504436 | 724.3314104 | Ugt3a1 |
| ENSMUSG00000072692 | 73.51960895 | 70.98175747 | 58.72957382 | Rpl37rt |
| ENSMUSG00000072849 | 570.0433447 | 342.3825948 | 378.8057511 | Serpina1e |
| ENSMUSG00000072999 | 294.0784358 | 417.5397498 | 310.287915 | Gm15401 |
| ENSMUSG00000073460 | 51.14407579 | 79.33255246 | 37.19539675 | Pnldc1 |
| ENSMUSG00000073835 | 85.24012632 | 69.93790809 | 16.64004591 | Mup-ps12 |
| ENSMUSG00000074024 | 118.2706753 | 117.9549793 | 98.86144926 | 4632427E13Rik |
| ENSMUSG00000074063 | 2843.823714 | 6567.900264 | 5870.020903 | Osgin1 |
| ENSMUSG00000074213 | 24.50653632 | 27.14008374 | 18.59769837 | Gm10642 |
| ENSMUSG00000074345 | 26.63753947 | 24.00853561 | 29.36478691 | Tnfaip8l3 |
| ENSMUSG00000074375 | 860.9252758 | 876.8334746 | 855.4941252 | Sult2a3 |
| ENSMUSG00000074876 | 71.38860579 | 41.75397498 | 54.81426889 | Spata5l1 |
| ENSMUSG00000075470 | 77.78161526 | 80.37640184 | 251.5583412 | Deaf1 |
| ENSMUSG00000075552 | 1023.947017 | 947.815232 | 894.6471745 | Cyp3a41b |
| ENSMUSG00000075590 | 1682.426993 | 1456.169877 | 1087.475942 | Nrbp2 |
| ENSMUSG00000076490 | 35.16155211 | 20.87698749 | 18.59769837 | Trbc1 |
| ENSMUSG00000076569 | 72.45410737 | 110.6480337 | 183.0405051 | Igkv5-39 |
| ENSMUSG00000076596 | 152.3667258 | 28.18393311 | 26.42830822 | Igkv3-10 |
| ENSMUSG00000076609 | 4135.211628 | 4467.675323 | 1384.06029 | Igkc |
| ENSMUSG00000076613 | 100.1571484 | 155.5335568 | 99.84027549 | Ighg2b |
| ENSMUSG00000076617 | 5037.691465 | 5276.658588 | 2480.345667 | Ighm |
| ENSMUSG00000076934 | 77.78161526 | 36.53472811 | 58.72957382 | Iglv1 |
| ENSMUSG00000077148 | 9.589514211 | 19.83313812 | 11.74591476 | Gm22935 |
| ENSMUSG00000078193 | 42.62006316 | 35.49087873 | 30.34361314 | Gm2000 |
| ENSMUSG00000078234 | 197.1177921 | 263.0500424 | 351.3986167 | Klhdc7a |
| ENSMUSG00000078650 | 6922.563759 | 10279.82864 | 10371.64274 | G6pc |
| ENSMUSG00000078651 | 55.40608211 | 57.4117156 | 43.06835413 | Aoc2 |
| ENSMUSG00000078672 | 337.7640005 | 374.7419254 | 237.854774 | Mup20 |
| ENSMUSG00000078688 | 49.01307263 | 38.62242686 | 39.15304921 | Mup2 |
| ENSMUSG00000078817 | 204.5763032 | 280.7954817 | 342.5891806 | Nlrp12 |
| ENSMUSG00000079017 | 145.9737163 | 124.2180756 | 88.09436072 | Ifi27l2a |
| ENSMUSG00000079036 | 575.3708526 | 288.1024274 | 180.1040264 | Alkbh1 |
| ENSMUSG00000079065 | 371.8600511 | 353.864938 | 194.7864198 | BC005561 |
| ENSMUSG00000079465 | 46.88206947 | 56.36786622 | 36.21657052 | Col4a3 |
| ENSMUSG00000079470 | 201.3797984 | 169.1035987 | 244.7065576 | Utp14b |
| ENSMUSG00000080059 | 37.29255526 | 13.57004187 | 19.57652461 | Rps19-ps3 |
| ENSMUSG00000081344 | 64.99559632 | 55.32401685 | 43.06835413 | Gm14303 |
| ENSMUSG00000082065 | 60.73359 | 39.66627623 | 5.872957382 | Mup-ps14 |
| ENSMUSG00000082173 | 41.55456158 | 31.31548123 | 8.809436072 | Mup-ps10 |
| ENSMUSG00000082586 | 15.98252368 | 7.306945621 | 17.61887214 | Sult2a-ps1 |
| ENSMUSG00000082658 | 85.24012632 | 129.4373224 | 49.92013774 | Fau-ps2 |
| ENSMUSG00000083327 | 36.22705368 | 34.44702936 | 66.56018366 | Vcp-rs |
| ENSMUSG00000083621 | 54.34058053 | 48.01707123 | 31.32243937 | Gm14586 |
| ENSMUSG00000083716 | 70.32310421 | 86.63949808 | 39.15304921 | Gm13436 |
| ENSMUSG00000083813 | 86.3056279 | 84.55179933 | 92.00966564 | Gm15502 |
| ENSMUSG00000083863 | 64.99559632 | 54.28016747 | 47.96248528 | Gm13341 |
| ENSMUSG00000083992 | 39.42355842 | 40.71012561 | 29.36478691 | Gm11478 |
| ENSMUSG00000084822 | 66.0610979 | 91.85874496 | 58.72957382 | Myadml2os |
| ENSMUSG00000084883 | 24.50653632 | 30.27163186 | 63.62370497 | Ccdc85c |
| ENSMUSG00000085001 | 156.6287321 | 70.98175747 | 70.47548858 | Rapgef4os2 |
| ENSMUSG00000085156 | 157.6942337 | 59.49941435 | 34.25891806 | Snhg15 |
| ENSMUSG00000085445 | 126.7946879 | 143.0073643 | 187.9346362 | Gm16348 |
| ENSMUSG00000085834 | 1120.907661 | 744.264604 | 450.2600659 | Gm15622 |
| ENSMUSG00000085995 | 444.3141584 | 772.4485371 | 450.2600659 | Gm2788 |
| ENSMUSG00000086140 | 11.72051737 | 22.96468624 | 25.44948199 | Hnf1aos2 |
| ENSMUSG00000086446 | 27.70304105 | 97.07799183 | 26.42830822 | Prkag2os1 |
| ENSMUSG00000086529 | 13.85152053 | 8.350794996 | 2.936478691 | Acss2os |
| ENSMUSG00000086786 | 2.131003158 | 3.131548123 | 15.66121968 | Gm15908 |
| ENSMUSG00000086844 | 73.51960895 | 42.79782435 | 46.98365905 | B230206H07Rik |
| ENSMUSG00000087382 | 414.4801142 | 369.5226786 | 445.3659348 | Ctcflos |
| ENSMUSG00000087445 | 61.79909158 | 54.28016747 | 39.15304921 | Gm14286 |
| ENSMUSG00000087595 | 41.55456158 | 62.63096247 | 36.21657052 | 1810012K08Rik |
| ENSMUSG00000087613 | 12.78601895 | 17.74543937 | 48.94131151 | Gm13855 |
| ENSMUSG00000087616 | 154.497729 | 34.44702936 | 26.42830822 | Gm14257 |
| ENSMUSG00000087658 | 36.22705368 | 28.18393311 | 23.49182953 | Hotairm1 |
| ENSMUSG00000089726 | 293.0129342 | 129.4373224 | 86.13670826 | Mir17hg |
| ENSMUSG00000089943 | 781.0126574 | 515.661591 | 1296.944755 | Ugt1a5 |
| ENSMUSG00000090021 | 15.98252368 | 39.66627623 | 48.94131151 | Gm6493 |
| ENSMUSG00000090145 | 154.497729 | 220.252218 | 375.8692724 | Ugt1a6b |
| ENSMUSG00000090175 | 125.7291863 | 209.8137243 | 409.1493642 | Ugt1a9 |
| ENSMUSG00000090264 | 57.53708526 | 80.37640184 | 52.85661643 | Eif4ebp3 |
| ENSMUSG00000090369 | 27.70304105 | 27.14008374 | 41.11070167 | 4933411K16Rik |
| ENSMUSG00000090555 | 1396.87257 | 624.2219259 | 491.3707676 | Gm8893 |
| ENSMUSG00000090610 | 35.16155211 | 28.18393311 | 22.5130033 | Gm3571 |
| ENSMUSG00000090698 | 247.1963663 | 60.54326372 | 76.34844596 | Apold1 |
| ENSMUSG00000091021 | 82.04362158 | 27.14008374 | 23.49182953 | Gm17300 |
| ENSMUSG00000091509 | 143.8427132 | 183.7174899 | 79.28492465 | Gm17066 |
| ENSMUSG00000092075 | 83.10912316 | 163.8843518 | 57.75074759 | Serpina4-ps1 |
| ENSMUSG00000094410 | 47.94757105 | 25.05238499 | 56.77192136 | Gm38394 |
| ENSMUSG00000095280 | 126.7946879 | 158.6651049 | 55.79309512 | Gm21738 |
| ENSMUSG00000095351 | 629.7114332 | 1051.15632 | 311.2667412 | Igkv3-2 |
| ENSMUSG00000096833 | 3.196504737 | 4.175397498 | 9.788262303 | Igkv4-55 |
| ENSMUSG00000096910 | 34.09605053 | 10.43849374 | 48.94131151 | Zfp955b |
| ENSMUSG00000096954 | 33.03054895 | 17.74543937 | 37.19539675 | Gdap10 |
| ENSMUSG00000097124 | 23.44103474 | 33.40317998 | 25.44948199 | A530020G20Rik |
| ENSMUSG00000097221 | 8.524012632 | 9.39464437 | 27.40713445 | 1810049J17Rik |
| ENSMUSG00000097312 | 114.0086689 | 161.796653 | 45.02600659 | Gm26870 |
| ENSMUSG00000097536 | 13.85152053 | 26.09623436 | 18.59769837 | 2610037D02Rik |
| ENSMUSG00000097615 | 69.25760263 | 65.76251059 | 70.47548858 | Gm2061 |
| ENSMUSG00000097660 | 60.73359 | 53.2363181 | 10.76708853 | Gm26762 |
| ENSMUSG00000097691 | 75.65061211 | 98.1218412 | 102.7767542 | 9030616G12Rik |
| ENSMUSG00000097743 | 155.5632305 | 98.1218412 | 88.09436072 | Gm16973 |
| ENSMUSG00000097908 | 52.20957737 | 45.92937248 | 57.75074759 | 4933404O12Rik |
| ENSMUSG00000097971 | 8345.008366 | 6440.550641 | 3776.311596 | Gm26917 |
| ENSMUSG00000097994 | 29.83404421 | 48.01707123 | 38.17422298 | Gm26982 |
| ENSMUSG00000098041 | 68.19210105 | 69.93790809 | 30.34361314 | Gm26981 |
| ENSMUSG00000098661 | 43.68556474 | 48.01707123 | 36.21657052 | Mir7052 |
| ENSMUSG00000098814 | 13.85152053 | 157.6212555 | 4.894131151 | Igkv19-93 |
| ENSMUSG00000098882 | 37.29255526 | 18.78928874 | 10.76708853 | Mir6392 |
| ENSMUSG00000099568 | 18.11352684 | 59.49941435 | 47.96248528 | Gm28513 |
| ENSMUSG00000099858 | 56.47158369 | 75.15715496 | 114.5226689 | Gm6652 |
| ENSMUSG00000100094 | 1017.554008 | 731.7384115 | 815.3622498 | 1810008l18Rik |
| ENSMUSG00000100468 | 6.393009474 | 6.263096247 | 9.788262303 | Tmem167-ps1 |
| ENSMUSG00000101939 | 52.20957737 | 56.36786622 | 46.00483282 | Gm28438 |
| ENSMUSG00000102275 | 56.47158369 | 54.28016747 | 18.59769837 | Gm37144 |
| ENSMUSG00000102577 | 137.4497037 | 252.6115486 | 199.680551 | Gm37969 |
| ENSMUSG00000102719 | 38.35805684 | 30.27163186 | 8.809436072 | Gm37760 |
| ENSMUSG00000102869 | 86.3056279 | 81.42025121 | 216.3205969 | 2900097C17Rik |
| ENSMUSG00000102882 | 26.63753947 | 38.62242686 | 15.66121968 | Gm2065 |
| ENSMUSG00000102918 | 18.11352684 | 14.61389124 | 27.40713445 | Pcdhgc3 |
| ENSMUSG00000103285 | 27.70304105 | 35.49087873 | 14.68239345 | Gm37274 |
| ENSMUSG00000103546 | 28.76854263 | 33.40317998 | 17.61887214 | Gm37666 |
| ENSMUSG00000104030 | 56.47158369 | 39.66627623 | 21.53417707 | 5330406M23Rik |
| ENSMUSG00000104388 | 46.88206947 | 34.44702936 | 12.72474099 | Gm37033 |
| ENSMUSG00000104399 | 77.78161526 | 41.75397498 | 42.0895279 | Gm37963 |
| ENSMUSG00000104445 | 38.35805684 | 40.71012561 | 25.44948199 | Rhbg |
| ENSMUSG00000104973 | 36.22705368 | 32.35933061 | 32.3012656 | A530041M06Rik |
| ENSMUSG00000105161 | 44.75106632 | 39.66627623 | 23.49182953 | Gm42595 |
| ENSMUSG00000105434 | 26.63753947 | 42.79782435 | 12.72474099 | Gm43359 |
| ENSMUSG00000105547 | 23.44103474 | 27.14008374 | 61.66605251 | Iglc3 |
| ENSMUSG00000105556 | 7.458511053 | 9.39464437 | 12.72474099 | Gm43080 |
| ENSMUSG00000105703 | 712.8205563 | 708.7737253 | 409.1493642 | Gm43305 |
| ENSMUSG00000105881 | 43.68556474 | 106.4726362 | 160.5275018 | 4932422M17Rik |
| ENSMUSG00000105906 | 182.20077 | 62.63096247 | 23.49182953 | Iglc1 |
| ENSMUSG00000106030 | 62.86459316 | 88.72719683 | 54.81426889 | Gm43611 |
| ENSMUSG00000106664 | 54.34058053 | 55.32401685 | 33.28009183 | Gm17936 |
| ENSMUSG00000106705 | 87.37112948 | 163.8843518 | 91.03083941 | Gm2602 |
| ENSMUSG00000106706 | 31.96504737 | 38.62242686 | 10.76708853 | C530043K16Rik |
| ENSMUSG00000106943 | 30.89954579 | 34.44702936 | 22.5130033 | Dancr |
| ENSMUSG00000107168 | 46.88206947 | 39.66627623 | 13.70356722 | Gm42507 |
| ENSMUSG00000107225 | 46.88206947 | 43.84167373 | 33.28009183 | Gm43637 |
| ENSMUSG00000107304 | 28.76854263 | 59.49941435 | 16.64004591 | Gm43775 |
| ENSMUSG00000107390 | 46.88206947 | 70.98175747 | 26.42830822 | Gm43323 |
| ENSMUSG00000107624 | 103.3536532 | 111.6918831 | 37.19539675 | Gm44005 |
| ENSMUSG00000108368 | 46.88206947 | 41.75397498 | 47.96248528 | Gm45053 |
| ENSMUSG00000108633 | 49.01307263 | 33.40317998 | 16.64004591 | Gm44694 |
| ENSMUSG00000108820 | 21.31003158 | 25.05238499 | 25.44948199 | Gm44620 |
| ENSMUSG00000108825 | 37.29255526 | 51.14861935 | 84.1790558 | Gm45838 |
| ENSMUSG00000109089 | 112.9431674 | 204.5944774 | 281.9019543 | 4833411C07Rik |
| ENSMUSG00000109115 | 4.262006316 | 4.175397498 | 8.809436072 | Gm44669 |
| ENSMUSG00000109157 | 25.5720379 | 30.27163186 | 18.59769837 | Gm44829 |
| ENSMUSG00000109262 | 14.91702211 | 19.83313812 | 12.72474099 | Gm44744 |
| ENSMUSG00000109291 | 17.04802526 | 38.62242686 | 25.44948199 | Gm2814 |
| ENSMUSG00000109536 | 109.7466626 | 90.81489558 | 68.51783612 | 9330162G02Rik |
| ENSMUSG00000109555 | 43.68556474 | 3.40317998 | 21.53417707 | Gm44891 |
| ENSMUSG00000109807 | 75.65061211 | 94.99029308 | 37.19539675 | Gm45244 |
| ENSMUSG00000109836 | 12.78601895 | 12.52619249 | 45.02600659 | Gm45884 |
| ENSMUSG00000109841 | 13.85152053 | 15.65774062 | 21.53417707 | E330011O21Rik |
| ENSMUSG00000110588 | 1747.42259 | 289.1462767 | 231.9818166 | Gm45774 |
| ENSMUSG00000110613 | 26.63753947 | 16.70158999 | 28.38596068 | Lncbate1 |
| ENSMUSG00000110702 | 45.8165679 | 30.27163186 | 25.44948199 | Gm45767 |
| ENSMUSG00000110755 | 79.91261842 | 28.18393311 | 20.55535084 | BC049987 |
| ENSMUSG00000111282 | 87.37112948 | 62.63096247 | 27.40713445 | Gm47528 |
| ENSMUSG00000111312 | 13.85152053 | 20.87698749 | 22.5130033 | Gm47205 |
| ENSMUSG00000111631 | 28.76854263 | 66.80635997 | 52.85661643 | Gm32017 |
| ENSMUSG00000111709 | 12.78601895 | 8.350794996 | 5.872957382 | Gm3776 |
| ENSMUSG00000111774 | 5.327507895 | 7.306945621 | 1.957652461 | AC166078.1 |

TABLE 4
RNA-seq analysis of differentially expressed genes between the PBS (Control
Group) or TGFRt15-TGFRs (TGFRt15-TGFRs group) in aged mice liver

| ID | log2FoldChange | pvalue | padj | agedliver92181tox | agedliver92182tox | agedliver92183tox |
|---|---|---|---|---|---|---|
| ENSMUSG00000000204 | −2.005879896 | 3.88E−05 | 0.000867087 | 8.974563618 | 11.65468178 | 11.93770314 |
| ENSMUSG00000000317 | −1.462090887 | 1.76E−06 | 7.11E−05 | 60.82759785 | 43.70505667 | 55.0970914 |
| ENSMUSG00000000686 | 1.151464201 | 2.84E−06 | 0.000104278 | 355.9910235 | 571.0794072 | 469.2435618 |
| ENSMUSG00000001227 | −1.099312542 | 1.49E−06 | 6.21E−05 | 100.7145473 | 67.98564371 | 92.74677053 |
| ENSMUSG00000001403 | −1.612087212 | 0.002110809 | 0.017174878 | 16.9519535 | 6.798564371 | 11.01941828 |
| ENSMUSG00000001983 | 1.013926416 | 1.20E−05 | 0.000332241 | 308.1266842 | 343.8131125 | 299.3608633 |
| ENSMUSG00000002233 | −1.270303057 | 1.05E−10 | 1.46E−08 | 133.6212805 | 104.892136 | 141.4158679 |
| ENSMUSG00000002250 | −1.142106413 | 9.77E−06 | 0.000282853 | 303.1408155 | 156.3669805 | 415.9830401 |
| ENSMUSG00000002289 | −2.433615292 | 5.35E−35 | 1.82E−31 | 340.0362437 | 320.5037489 | 528.0137926 |
| ENSMUSG00000002831 | −2.428567233 | 6.40E−14 | 1.90E−11 | 67.807814 | 71.87053764 | 51.42395197 |
| ENSMUSG00000003032 | −1.814709673 | 6.30E−10 | 7.35E−08 | 33.903907 | 28.16548097 | 44.99595798 |
| ENSMUSG00000003348 | 1.035062446 | 0.006673687 | 0.038727262 | 47.86433929 | 51.47484453 | 59.68851569 |
| ENSMUSG00000003500 | −1.169914769 | 9.39E−05 | 0.001721093 | 55.84172918 | 53.41729149 | 52.34223683 |
| ENSMUSG00000003541 | −2.579058324 | 2.05E−08 | 1.65E−06 | 9.971737353 | 11.65468178 | 11.01941828 |
| ENSMUSG00000003848 | −1.114660438 | 1.53E−05 | 0.000400378 | 168.5223613 | 191.3310259 | 135.9061588 |
| ENSMUSG00000004100 | −2.152000208 | 2.40E−05 | 0.000578522 | 177.4969249 | 181.6187911 | 165.2912742 |
| ENSMUSG00000004933 | −1.661199536 | 0.00641801 | 0.037574902 | 11.96608482 | 8.741011335 | 3.673139427 |
| ENSMUSG00000004951 | −1.245761383 | 7.16E−05 | 0.001407433 | 177.4969249 | 194.2446963 | 114.7856071 |
| ENSMUSG00000005148 | −1.641216558 | 0.00407345 | 0.027163637 | 8.974563618 | 3.884893927 | 9.182848567 |
| ENSMUSG00000005547 | 1.316837452 | 9.30E−15 | 3.02E−12 | 9129.125547 | 13948.71164 | 12101.15784 |
| ENSMUSG00000005580 | 1.4493211 | 2.75E−08 | 2.14E−06 | 248.2962601 | 371.00737 | 337.0105424 |
| ENSMUSG00000006050 | −1.042916307 | 2.32E−07 | 1.27E−05 | 992.1878666 | 1015.899762 | 869.6157593 |
| ENSMUSG00000006134 | 1.201348738 | 1.44E−09 | 1.52E−07 | 677.0809663 | 610.8995699 | 678.6125091 |
| ENSMUSG00000006517 | 1.182376585 | 1.39E−07 | 8.18E−06 | 2010.30225 | 1472.374798 | 1714.437827 |
| ENSMUSG00000006587 | 1.922052303 | 0.003206819 | 0.022932036 | 30.91238579 | 14.56835222 | 21.1205517 |
| ENSMUSG00000006711 | 1.246016282 | 4.07E−11 | 5.91E−09 | 435.7649223 | 410.8275327 | 410.4733309 |
| ENSMUSG00000006777 | 1.982368887 | 0.000106088 | 0.001901083 | 22.93499591 | 53.41729149 | 33.05825484 |
| ENSMUSG00000008153 | 1.444860417 | 1.72E−08 | 1.42E−06 | 278.2114721 | 339.9282186 | 185.4935411 |
| ENSMUSG00000009013 | −1.025154388 | 6.49E−07 | 3.04E−05 | 138.6071492 | 163.1655449 | 192.8398199 |
| ENSMUSG00000009633 | −1.150697898 | 7.41E−07 | 3.38E−05 | 1177.662181 | 1721.008009 | 1060.619009 |
| ENSMUSG00000014547 | 1.47853344 | 5.88E−06 | 0.00018548 | 76.78237762 | 107.8058065 | 108.3576131 |
| ENSMUSG00000014609 | 1.198939285 | 0.004891473 | 0.030982358 | 34.90108074 | 33.99282186 | 34.89482455 |
| ENSMUSG00000015224 | −1.47719251 | 7.85E−07 | 3.55E−05 | 57.83607665 | 125.2878291 | 79.89078253 |
| ENSMUSG00000015312 | −2.170653759 | 1.83E−07 | 1.04E−05 | 27.92086459 | 16.51079919 | 10.10113342 |
| ENSMUSG00000016128 | 1.194768611 | 1.81E−09 | 1.86E−07 | 486.6207828 | 535.1441384 | 631.7799814 |
| ENSMUSG00000016356 | −1.303561468 | 6.94E−06 | 0.00021464 | 50.8558605 | 59.24463238 | 46.83252769 |
| ENSMUSG00000017737 | −1.495519922 | 0.005185903 | 0.032307064 | 19.94347471 | 7.769787853 | 5.50970914 |
| ENSMUSG00000017868 | 1.520950393 | 2.42E−16 | 1.03E−13 | 1574.537328 | 1919.1376 | 1179.077756 |
| ENSMUSG00000018486 | 1.561119026 | 0.006908525 | 0.039768921 | 26.92369085 | 21.3669166 | 27.5485457 |
| ENSMUSG00000019082 | −1.214544808 | 1.93E−12 | 4.05E−10 | 2583.677148 | 3303.131061 | 2721.796315 |
| ENSMUSG00000019726 | 1.436923585 | 3.39E−10 | 4.31E−08 | 430.7790536 | 552.626161 | 627.1885571 |
| ENSMUSG00000019737 | −1.672592181 | 0.00414668 | 0.027410702 | 8.974563618 | 9.712234816 | 6.427993997 |
| ENSMUSG00000019883 | 1.383035463 | 1.73E−09 | 1.79E−07 | 790.7587721 | 685.683778 | 707.0793397 |
| ENSMUSG00000020018 | −1.224865075 | 5.55E−07 | 2.68E−05 | 76.78237762 | 100.0360186 | 117.5404617 |
| ENSMUSG00000020027 | −2.148540831 | 0.000201757 | 0.00309142 | 308.1266842 | 206.8706016 | 291.0962996 |
| ENSMUSG00000020091 | 1.323362559 | 0.005934886 | 0.035409378 | 1482.797344 | 1694.784975 | 1814.530877 |
| ENSMUSG00000020122 | −1.730853584 | 6.04E−21 | 5.49E−18 | 534.4851221 | 692.4823424 | 689.6319274 |
| ENSMUSG00000020335 | 1.099463556 | 0.002629472 | 0.020167667 | 41.88129688 | 88.38133683 | 80.80906739 |
| ENSMUSG00000020429 | −1.914789163 | 0.005628577 | 0.034083881 | 1082.930677 | 219.4965068 | 977.0550875 |
| ENSMUSG00000020441 | −1.319584205 | 1.17E−07 | 7.17E−06 | 96.72585232 | 114.6043708 | 87.23706139 |
| ENSMUSG00000020532 | 1.125678465 | 6.99E−06 | 0.000215157 | 6805.710743 | 2936.008585 | 5607.047335 |
| ENSMUSG00000020641 | −1.035196027 | 2.26E−05 | 0.00055792 | 307.1295105 | 181.6187911 | 139.5792982 |
| ENSMUSG00000020656 | −2.17958622 | 1.43E−08 | 1.21E−06 | 16.9519535 | 7.769787853 | 23.87540627 |
| ENSMUSG00000020681 | 1.270736177 | 0.003452779 | 0.024158824 | 30.91238579 | 35.93526882 | 52.34223683 |
| ENSMUSG00000020692 | −1.373008874 | 7.68E−06 | 0.000232298 | 53.84738171 | 65.07197327 | 76.21764311 |
| ENSMUSG00000020812 | −1.318469089 | 0.000692163 | 0.007551219 | 32.90673326 | 43.70505667 | 47.75081255 |
| ENSMUSG00000020889 | 1.857219295 | 5.65E−19 | 3.50E−16 | 1005.151125 | 1714.209445 | 1803.511459 |
| ENSMUSG00000020917 | 1.579449336 | 7.98E−12 | 1.40E−09 | 34685.69121 | 17873.42573 | 22782.64729 |
| ENSMUSG00000020948 | 1.248898073 | 0.003935164 | 0.026500655 | 67.807814 | 53.41729149 | 56.93366111 |
| ENSMUSG00000020961 | 1.570215029 | 7.42E−07 | 3.38E−05 | 116.669327 | 116.5468178 | 141.4158679 |
| ENSMUSG00000021250 | −3.196150425 | 1.40E−18 | 7.95E−16 | 14.95760603 | 8.741011335 | 25.71197599 |
| ENSMUSG00000021260 | 1.563177947 | 0.002093122 | 0.017081929 | 31.90955953 | 29.13670445 | 23.87540627 |
| ENSMUSG00000021416 | 1.171425747 | 1.31E−08 | 1.13E−06 | 910.4196203 | 675.9715432 | 629.0251268 |
| ENSMUSG00000021453 | −2.706070458 | 5.59E−23 | 6.28E−20 | 149.5760603 | 61.18707934 | 130.3964497 |
| ENSMUSG00000021611 | 1.027773708 | 1.21E−09 | 1.30E−07 | 416.8186214 | 423.453438 | 475.6715558 |
| ENSMUSG00000021670 | 1.206582161 | 2.43E−07 | 1.30E−05 | 4843.272832 | 4623.994996 | 5239.733392 |
| ENSMUSG00000021684 | −1.138260866 | 0.001182935 | 0.011115132 | 32.90673326 | 25.25181052 | 22.03883656 |
| ENSMUSG00000021773 | −1.294178405 | 0.002327317 | 0.01846284 | 17.94912724 | 12.62590526 | 22.03883656 |
| ENSMUSG00000021775 | 1.550962326 | 2.41E−10 | 3.16E−08 | 1168.687618 | 1405.360378 | 1119.38924 |
| ENSMUSG00000021804 | 1.339955479 | 0.00532128 | 0.032924815 | 26.92369085 | 35.93526882 | 37.64967912 |
| ENSMUSG00000021958 | −1.041099878 | 0.007096785 | 0.040595156 | 25.92651712 | 21.3669166 | 47.75081255 |
| ENSMUSG00000022383 | 1.183413475 | 3.19E−08 | 2.41E−06 | 1160.710228 | 1260.648079 | 1669.441869 |
| ENSMUSG00000022389 | 1.619123522 | 2.95E−16 | 1.22E−13 | 1357.153454 | 2316.368004 | 1561.084256 |
| ENSMUSG00000022408 | 1.528188008 | 2.54E−06 | 9.66E−05 | 74.78803015 | 107.8058065 | 82.6456371 |
| ENSMUSG00000022528 | −1.557702695 | 1.66E−19 | 1.13E−16 | 205.4177895 | 185.503685 | 197.4312442 |
| ENSMUSG00000022651 | −4.171544663 | 2.34E−09 | 2.36E−07 | 3.988694941 | 2.913670445 | 0 |
| ENSMUSG00000022704 | −1.016808687 | 0.000177103 | 0.002808323 | 94.73150485 | 108.7770299 | 100.0930494 |
| ENSMUSG00000022853 | 1.04479536 | 2.29E−11 | 3.59E−09 | 9525.00352 | 10073.52995 | 7460.146176 |
| ENSMUSG00000022883 | 1.330834067 | 1.00E−09 | 1.09E−07 | 280.2058196 | 336.0433246 | 355.3762395 |
| ENSMUSG00000022887 | 1.363326654 | 1.68E−07 | 9.74E−06 | 1196.608482 | 1241.22361 | 1538.127135 |
| ENSMUSG00000022911 | 1.024722285 | 3.96E−05 | 0.000880701 | 133.6212805 | 188.4173554 | 193.7581048 |
| ENSMUSG00000023034 | −2.875895339 | 0.000200077 | 0.003084527 | 58.83325038 | 34.96404534 | 22.95712142 |
| ENSMUSG00000023044 | −1.350199698 | 7.38E−12 | 1.31E−09 | 1875.683796 | 2461.080302 | 2315.914409 |
| ENSMUSG00000023052 | −1.334599319 | 0.004914148 | 0.031082668 | 13.96043229 | 11.65468178 | 17.44741228 |
| ENSMUSG00000023067 | −3.022867976 | 2.66E−57 | 3.63E−53 | 136.6128017 | 95.1799012 | 134.9878739 |
| ENSMUSG00000023073 | 1.225822592 | 2.02E−05 | 0.00050363 | 443.7423122 | 525.4319036 | 592.2937326 |
| ENSMUSG00000023341 | −1.14606456 | 1.95E−06 | 7.71E−05 | 89.74563618 | 99.06479513 | 85.40049167 |
| ENSMUSG00000023571 | −1.317554169 | 0.002212852 | 0.017761424 | 18.94630097 | 21.3669166 | 14.69255771 |
| ENSMUSG00000023800 | 1.312092825 | 2.45E−12 | 4.99E−10 | 459.697092 | 410.8275327 | 456.3875738 |
| ENSMUSG00000023905 | −1.475845141 | 9.44E−06 | 0.000275028 | 54.84455544 | 95.1799012 | 81.72735225 |
| ENSMUSG00000023927 | −1.218211099 | 0.003077602 | 0.022366209 | 27.92086459 | 26.223034 | 12.85598799 |
| ENSMUSG00000023968 | −1.267291327 | 0.008148531 | 0.04468095 | 10.96891109 | 11.65468178 | 15.61084256 |
| ENSMUSG00000024118 | −1.757053931 | 5.31E−18 | 2.79E−15 | 442.7451385 | 592.4463238 | 352.621385 |
| ENSMUSG00000024130 | 1.042830636 | 1.30E−05 | 0.000354612 | 2989.526858 | 2745.648783 | 3530.805274 |
| ENSMUSG00000024136 | −1.225506878 | 0.002002269 | 0.016518413 | 27.92086459 | 19.42446963 | 17.44741228 |
| ENSMUSG00000024190 | −1.579039564 | 2.17E−13 | 5.48E−11 | 281.2029934 | 502.12254 | 460.0607132 |
| ENSMUSG00000024236 | 1.389584652 | 9.49E−13 | 2.12E−10 | 1344.190195 | 1041.151572 | 1299.373072 |
| ENSMUSG00000024411 | 1.68012077 | 5.07E−05 | 0.001068968 | 63.81911906 | 48.56117408 | 35.81310941 |
| ENSMUSG00000024440 | 1.007637941 | 0.000827422 | 0.008613399 | 107.6947634 | 103.9209125 | 147.8438619 |
| ENSMUSG00000024665 | 1.18951247 | 3.11E−12 | 6.05E−10 | 9244.7977 | 8635.147975 | 9906.457034 |
| ENSMUSG00000024843 | −1.175648724 | 2.45E−11 | 3.79E−09 | 1421.969747 | 1016.870985 | 1263.559963 |
| ENSMUSG00000024887 | 1.067323547 | 1.55E−06 | 6.39E−05 | 398.8694941 | 436.0793433 | 565.6634717 |
| ENSMUSG00000024924 | 1.265415535 | 2.60E−12 | 5.20E−10 | 642.1798855 | 816.7989481 | 589.538878 |
| ENSMUSG00000024970 | −1.174479585 | 0.000322102 | 0.004379373 | 31.90955953 | 39.82016275 | 25.71197599 |
| ENSMUSG00000024978 | 2.527860907 | 4.51E−08 | 3.23E−06 | 10405.50793 | 5475.757989 | 8311.396238 |
| ENSMUSG00000025003 | 1.045766059 | 5.74E−07 | 2.75E−05 | 301.1464681 | 286.5109271 | 410.4733309 |
| ENSMUSG00000025006 | 1.229102652 | 2.44E−06 | 9.41E−05 | 288.1832095 | 294.2807149 | 416.9013249 |
| ENSMUSG00000025153 | 1.113433094 | 9.44E−07 | 4.18E−05 | 85604.37365 | 41744.15646 | 57005.28733 |
| ENSMUSG00000025161 | −1.470478397 | 0.001962044 | 0.016285081 | 18.94630097 | 9.712234816 | 14.69255771 |
| ENSMUSG00000025240 | 1.333245056 | 1.72E−06 | 6.99E−05 | 553.4314231 | 682.7701076 | 696.0599214 |
| ENSMUSG00000025323 | 1.252220218 | 7.14E−07 | 3.28E−05 | 138.6071492 | 127.2302761 | 172.6375531 |
| ENSMUSG00000025402 | −1.340941683 | 6.59E−15 | 2.25E−12 | 217.3838743 | 245.7195409 | 214.8786565 |
| ENSMUSG00000025429 | 1.693186246 | 2.17E−13 | 5.48E−11 | 624.2307583 | 540.0002558 | 651.9822483 |
| ENSMUSG00000025450 | −1.257708301 | 2.83E−05 | 0.000669372 | 30.91238579 | 62.15830282 | 55.0970914 |
| ENSMUSG00000025997 | −1.284615532 | 0.009041141 | 0.048049118 | 26.92369085 | 9.712234816 | 12.85598799 |
| ENSMUSG00000026020 | −1.156643623 | 9.59E−08 | 6.11E−06 | 250.2906076 | 294.2807149 | 269.9757479 |
| ENSMUSG00000026249 | −1.326755053 | 0.000451208 | 0.005606296 | 36.89542821 | 50.50362104 | 26.63026084 |
| ENSMUSG00000026358 | −2.793399328 | 5.66E−13 | 1.33E−10 | 12.96325856 | 11.65468178 | 12.85598799 |
| ENSMUSG00000026398 | 1.137826301 | 6.48E−12 | 1.16E−09 | 1403.023446 | 1667.590718 | 1539.04542 |
| ENSMUSG00000026471 | 1.000583106 | 1.46E−05 | 0.000384933 | 269.2369085 | 237.949753 | 315.8899907 |
| ENSMUSG00000026475 | −1.56210025 | 0.004964709 | 0.031300593 | 735.9142167 | 388.4893927 | 994.5024998 |
| ENSMUSG00000026525 | −1.790734538 | 0.001947167 | 0.0161813 | 7.977389882 | 8.741011335 | 6.427993997 |
| ENSMUSG00000026822 | −1.02651459 | 5.14E−06 | 0.00016724 | 122.6523694 | 119.4604882 | 110.1941828 |
| ENSMUSG00000026826 | −1.483020728 | 0.008821676 | 0.047306802 | 15.95477976 | 10.6834583 | 11.93770314 |
| ENSMUSG00000026832 | −1.174113684 | 0.001522033 | 0.013425591 | 30.91238579 | 34.96404534 | 25.71197599 |
| ENSMUSG00000027360 | −1.979964656 | 1.70E−12 | 3.69E−10 | 54.84455544 | 93.23745424 | 72.54450368 |
| ENSMUSG00000027398 | −1.057691022 | 0.001937845 | 0.016123489 | 78.77672509 | 37.87771578 | 32.13996998 |
| ENSMUSG00000027405 | −1.241634744 | 7.40E−06 | 0.000226395 | 225.3612642 | 254.4605522 | 248.8551962 |
| ENSMUSG00000027496 | −1.007831872 | 0.004782077 | 0.030501955 | 38.88977568 | 37.87771578 | 17.44741228 |
| ENSMUSG00000027513 | −1.115800194 | 1.04E−10 | 1.46E−08 | 16805.36896 | 18645.5484 | 18901.97549 |
| ENSMUSG00000027605 | 1.86697993 | 9.67E−16 | 3.77E−13 | 27327.54622 | 18714.50527 | 21561.32844 |
| ENSMUSG00000027762 | 1.17286147 | 2.98E−15 | 1.13E−12 | 1319.260852 | 1520.935972 | 1404.975831 |
| ENSMUSG00000027907 | −1.140239221 | 0.000303295 | 0.004203282 | 47.86433929 | 56.33096193 | 106.5210434 |
| ENSMUSG00000027947 | −1.05537492 | 2.15E−06 | 8.45E−05 | 434.7677486 | 468.1297181 | 461.8972829 |
| ENSMUSG00000028008 | 1.245889 | 0.000553467 | 0.00652119 | 54.84455544 | 81.58277246 | 55.0970914 |
| ENSMUSG00000028339 | 1.011959422 | 2.14E−08 | 1.71E−06 | 706.9961783 | 505.0362104 | 521.5857986 |
| ENSMUSG00000028445 | 1.767301879 | 0.003925136 | 0.026446185 | 70.79933521 | 71.87053764 | 91.82848567 |
| ENSMUSG00000028630 | 1.331166045 | 1.46E−07 | 8.59E−06 | 373.9401507 | 358.3814647 | 406.8001915 |
| ENSMUSG00000028838 | 1.693729472 | 3.36E−09 | 3.22E−07 | 304.1379893 | 151.5108631 | 198.349529 |
| ENSMUSG00000028859 | −1.001034699 | 0.004539011 | 0.029294129 | 74.78803015 | 57.30218542 | 29.38511541 |
| ENSMUSG00000028862 | −2.278242573 | 1.62E−07 | 9.45E−06 | 8.974563618 | 9.712234816 | 17.44741228 |
| ENSMUSG00000028864 | 1.08474786 | 0.000792949 | 0.008337279 | 131.6269331 | 134.0288405 | 203.8592382 |
| ENSMUSG00000028957 | 2.966674755 | 1.69E−27 | 3.84E−24 | 314.1097266 | 609.9283465 | 341.6019667 |
| ENSMUSG00000028976 | 1.532387058 | 3.09E−07 | 1.58E−05 | 157.5534502 | 94.20867772 | 101.9296191 |
| ENSMUSG00000029086 | 1.631659452 | 1.01E−18 | 6.00E−16 | 713.9763945 | 617.6981343 | 593.2120174 |
| ENSMUSG00000029135 | −1.266157753 | 3.55E−07 | 1.81E−05 | 179.4912724 | 127.2302761 | 136.8244436 |
| ENSMUSG00000029188 | −2.354954255 | 1.89E−12 | 4.02E−10 | 42.87847062 | 12.62590526 | 51.42395197 |
| ENSMUSG00000029195 | 1.272024305 | 6.31E−09 | 5.74E−07 | 705.0018309 | 838.1658646 | 788.8066919 |
| ENSMUSG00000029370 | 1.683633222 | 1.05E−19 | 7.55E−17 | 546.4512069 | 472.9858356 | 579.4377446 |
| ENSMUSG00000029373 | −1.650047987 | 0.002725238 | 0.020577678 | 7.977389882 | 5.82734089 | 9.182848567 |
| ENSMUSG00000029380 | −2.803403219 | 8.38E−26 | 1.43E−22 | 53.84738171 | 128.2014996 | 71.62621882 |
| ENSMUSG00000029580 | −1.162469269 | 9.11E−12 | 1.55E−09 | 8321.414821 | 7155.974613 | 6927.540959 |
| ENSMUSG00000029591 | −1.593385799 | 3.01E−08 | 2.29E−06 | 43.87564435 | 60.21585586 | 35.81310941 |
| ENSMUSG00000029656 | −1.019716092 | 0.000134204 | 0.002299171 | 189.4630097 | 409.8563092 | 189.1666805 |
| ENSMUSG00000030032 | −1.061169015 | 0.007631272 | 0.042668167 | 23.93216965 | 14.56835222 | 25.71197599 |
| ENSMUSG00000030055 | 1.305889925 | 4.54E−06 | 0.000151075 | 239.3216965 | 263.2015635 | 292.9328693 |
| ENSMUSG00000030691 | 1.035229834 | 1.01E−08 | 8.79E−07 | 365.9627609 | 443.8491311 | 415.0647552 |
| ENSMUSG00000030782 | −1.087204324 | 0.00053618 | 0.006369234 | 38.88977568 | 39.82016275 | 40.40453369 |
| ENSMUSG00000030814 | −1.357189589 | 8.78E−10 | 9.81E−08 | 261.2595186 | 283.5972566 | 291.0962996 |
| ENSMUSG00000030827 | −1.631143551 | 1.45E−05 | 0.000383464 | 26.92369085 | 20.39569311 | 33.05825484 |
| ENSMUSG00000030934 | 1.434845811 | 0.000702548 | 0.007652274 | 13671.25191 | 24780.76713 | 9134.17947 |
| ENSMUSG00000030968 | −1.140744433 | 0.000133867 | 0.002296287 | 34.90108074 | 72.84176112 | 54.17880654 |
| ENSMUSG00000031010 | 1.281779593 | 3.26E−07 | 1.66E−05 | 953.2980909 | 1398.561814 | 1420.586673 |
| ENSMUSG00000031271 | −1.719181951 | 2.91E−23 | 3.97E−20 | 339.03907 | 470.0721651 | 364.5590881 |
| ENSMUSG00000031378 | 1.154152634 | 6.32E−15 | 2.21E−12 | 1014.125689 | 912.9500727 | 885.2266019 |
| ENSMUSG00000031465 | −1.254345174 | 0.0029265 | 0.021654196 | 21.93782218 | 11.65468178 | 20.20226685 |
| ENSMUSG00000031762 | −4.701992063 | 1.08E−05 | 0.000305681 | 384.9090618 | 188.4173554 | 32.13996998 |
| ENSMUSG00000031765 | −4.008275951 | 6.98E−08 | 4.64E−06 | 1129.797842 | 691.5111189 | 337.0105424 |
| ENSMUSG00000032009 | 1.096135097 | 4.98E−08 | 3.50E−06 | 816.6852892 | 873.12991 | 1044.089882 |
| ENSMUSG00000032064 | 1.076164714 | 3.75E−10 | 4.69E−08 | 659.131839 | 690.5398954 | 661.1650968 |
| ENSMUSG00000032083 | −1.056710751 | 2.91E−09 | 2.86E−07 | 57416.2665 | 56826.28591 | 68671.17815 |
| ENSMUSG00000032091 | 2.476316256 | 2.69E−06 | 0.000101175 | 102.7088947 | 50.50362104 | 32.13996998 |
| ENSMUSG00000032285 | 1.014640865 | 0.008899206 | 0.047498424 | 51.85303424 | 70.89931416 | 52.34223683 |
| ENSMUSG00000032417 | 1.412452049 | 0.000231229 | 0.003394264 | 102.7088947 | 50.50362104 | 80.80906739 |
| ENSMUSG00000032418 | 1.488424738 | 1.96E−09 | 2.00E−07 | 17569.20404 | 7712.485668 | 13799.06654 |
| ENSMUSG00000032500 | 2.09734091 | 1.65E−20 | 1.25E−17 | 413.8271001 | 367.1224761 | 372.8236518 |
| ENSMUSG00000032561 | 1.808480512 | 0.000942567 | 0.009458265 | 1361.142149 | 660.4319675 | 1176.322901 |
| ENSMUSG00000032702 | 1.072973429 | 8.45E−08 | 5.48E−06 | 1328.235415 | 1308.23803 | 1494.049462 |
| ENSMUSG00000032724 | 1.34458697 | 3.25E−10 | 4.18E−08 | 647.1657542 | 540.0002558 | 680.4490788 |
| ENSMUSG00000032735 | −1.26252931 | 1.21E−08 | 1.04E−06 | 191.4573572 | 257.3742226 | 294.769439 |
| ENSMUSG00000032786 | −1.26794906 | 3.89E−08 | 2.81E−06 | 1778.957944 | 2029.857077 | 2206.638511 |
| ENSMUSG00000032849 | 1.170479352 | 7.00E−10 | 8.09E−08 | 403.8553628 | 338.9569951 | 399.4539127 |
| ENSMUSG00000032860 | 1.046181292 | 6.20E−08 | 4.19E−06 | 339.03907 | 263.2015635 | 290.1780147 |
| ENSMUSG00000032883 | 1.139189345 | 1.88E−11 | 3.09E−09 | 1754.0286 | 1475.288469 | 1528.944286 |
| ENSMUSG00000033105 | 1.210153299 | 1.43E−10 | 1.97E−08 | 3593.814142 | 2741.763889 | 3240.627259 |
| ENSMUSG00000033594 | −1.045179866 | 5.30E−08 | 3.67E−06 | 379.9231931 | 342.841889 | 348.9482455 |
| ENSMUSG00000033624 | 1.545958738 | 3.43E−10 | 4.33E−08 | 290.177557 | 397.230404 | 428.8390281 |
| ENSMUSG00000033792 | 1.183185841 | 0.000561984 | 0.006589659 | 63.81911906 | 67.98564371 | 74.38107339 |
| ENSMUSG00000033855 | 1.291796081 | 0.000477464 | 0.005865923 | 117.6665008 | 69.92809068 | 198.349529 |
| ENSMUSG00000033967 | −2.822222129 | 4.36E−05 | 0.000952114 | 4.985868677 | 3.884893927 | 1.836569713 |
| ENSMUSG00000034066 | 1.2566902 | 2.38E−07 | 1.29E−05 | 275.2199509 | 346.7267829 | 310.3802816 |
| ENSMUSG00000034110 | 1.131917427 | 3.20E−05 | 0.000736514 | 99.71737353 | 110.7194769 | 115.7038919 |
| ENSMUSG00000034271 | −1.155793079 | 0.004733656 | 0.030334993 | 36.89542821 | 29.13670445 | 40.40453369 |
| ENSMUSG00000034755 | −1.51005032 | 0.006510973 | 0.037993212 | 7.977389882 | 5.82734089 | 12.85598799 |
| ENSMUSG00000034765 | −2.457798355 | 3.78E−06 | 0.000131464 | 23.93216965 | 12.62590526 | 9.182848567 |
| ENSMUSG00000034853 | 1.437062013 | 9.64E−14 | 2.63E−11 | 450.7225284 | 335.0721012 | 434.3487372 |
| ENSMUSG00000034926 | 1.093495822 | 1.31E−06 | 5.56E−05 | 17902.26007 | 19066.08817 | 17627.39611 |
| ENSMUSG00000035078 | 1.209517873 | 6.16E−05 | 0.001249206 | 322.0871165 | 384.6044987 | 346.193391 |
| ENSMUSG00000035112 | −1.146398835 | 0.004042609 | 0.02701081 | 23.93216965 | 14.56835222 | 18.36569713 |
| ENSMUSG00000035164 | 1.06213892 | 8.60E−06 | 0.000256206 | 131.6269331 | 166.0792154 | 141.4158679 |
| ENSMUSG00000035165 | −1.798528938 | 1.18E−06 | 5.05E−05 | 32.90673326 | 22.33814008 | 28.46683056 |
| ENSMUSG00000035284 | 1.432291641 | 4.22E−09 | 3.95E−07 | 725.9424793 | 940.1443302 | 1017.459621 |
| ENSMUSG00000035900 | 1.041020638 | 2.29E−08 | 1.81E−06 | 343.0277649 | 318.561302 | 367.3139427 |
| ENSMUSG00000035933 | 1.189062904 | 7.63E−07 | 3.47E−05 | 188.465836 | 221.4389538 | 244.2637719 |
| ENSMUSG00000035948 | 1.741049509 | 1.78E−10 | 2.38E−08 | 816.6852892 | 893.5256031 | 857.6780562 |
| ENSMUSG00000036062 | −1.708320866 | 0.004715534 | 0.030261525 | 7.977389882 | 9.712234816 | 3.673139427 |
| ENSMUSG00000036120 | −1.340248384 | 3.85E−09 | 3.64E−07 | 263.2538661 | 219.4965068 | 224.9797899 |
| ENSMUSG00000036611 | 1.04665245 | 4.08E−07 | 2.03E−05 | 846.6005013 | 616.7269108 | 585.8657386 |
| ENSMUSG00000037035 | −1.785829601 | 2.14E−10 | 2.84E−08 | 59.83042412 | 30.10792793 | 50.50566712 |
| ENSMUSG00000037071 | 1.600161226 | 3.89E−11 | 5.71E−09 | 372268.8876 | 280319.3774 | 340820.5063 |
| ENSMUSG00000037095 | −1.027049761 | 7.90E−10 | 8.97E−08 | 3492.102421 | 3177.843232 | 2991.772063 |
| ENSMUSG00000037157 | 1.742314913 | 8.42E−06 | 0.000251859 | 51.85303424 | 60.21585586 | 58.77023083 |
| ENSMUSG00000037336 | −1.65964734 | 0.000936801 | 0.009424872 | 14.95760603 | 7.769787853 | 8.26456371 |
| ENSMUSG00000037443 | −1.349128808 | 2.89E−12 | 5.72E−10 | 750.8718227 | 1078.058065 | 1089.08584 |
| ENSMUSG00000037447 | −1.380120078 | 0.000135203 | 0.002310483 | 30.91238579 | 23.30936356 | 25.71197599 |
| ENSMIJSG00000037465 | −1.250556349 | 2.44E−12 | 4.99E−10 | 342.0305912 | 272.9137983 | 297.5242936 |
| ENSMUSG00000037583 | −1.178511493 | 2.71E−06 | 0.000101645 | 181.4856198 | 270.0001279 | 213.9603716 |
| ENSMUSG00000037709 | 1.621036788 | 2.49E−19 | 1.62E−16 | 850.5891962 | 1019.784656 | 857.6780562 |
| ENSMUSG00000037887 | −1.887921138 | 0.000434734 | 0.005464027 | 15.95477976 | 8.741011335 | 11.01941828 |
| ENSMUSG00000038217 | 1.023783748 | 9.74E−10 | 1.07E−07 | 3279.704415 | 3214.749724 | 3049.624009 |
| ENSMUSG00000038233 | 1.132248981 | 2.75E−05 | 0.000651686 | 765.8294287 | 340.8994421 | 856.7597713 |
| ENSMUSG00000038253 | −2.17957789 | 5.49E−08 | 3.76E−06 | 19.94347471 | 18.45324615 | 14.69255771 |
| ENSMUSG00000038370 | −1.863884427 | 6.22E−10 | 7.32E−08 | 95.72867859 | 197.1583668 | 141.4158679 |
| ENSMUSG00000038415 | −1.598903332 | 5.74E−10 | 6.80E−08 | 144.5901916 | 136.9425109 | 205.6958079 |
| ENSMUSG00000038418 | −2.528561862 | 1.50E−17 | 7.58E−15 | 95.72867859 | 79.64032549 | 131.3147345 |
| ENSMUSG00000038473 | 1.076370271 | 0.000625033 | 0.007061263 | 117.6665008 | 63.12952631 | 89.99191596 |
| ENSMUSG00000038530 | −1.722746304 | 4.58E−05 | 0.00098969 | 17.94912724 | 16.51079919 | 13.77427285 |
| ENSMUSG00000038583 | −1.702074711 | 0.00088397 | 0.009002758 | 12.96325856 | 13.59712874 | 11.93770314 |
| ENSMUSG00000038587 | −1.31455808 | 3.12E−06 | 0.000113043 | 97.72302606 | 73.8129846 | 141.4158679 |
| ENSMUSG00000038751 | 1.774904606 | 6.46E−12 | 1.16E−09 | 167.5251875 | 125.2878291 | 171.7192682 |
| ENSMUSG00000038768 | 1.297337324 | 1.64E−05 | 0.000422782 | 329.0673326 | 229.2087417 | 124.8867405 |
| ENSMUSG00000038774 | 1.007283954 | 3.14E−06 | 0.00011311 | 450.7225284 | 497.2664226 | 488.5275438 |
| ENSMUSG00000038844 | 1.003328537 | 1.78E−06 | 7.13E−05 | 364.9655871 | 380.7196048 | 386.5979247 |
| ENSMUSG00000038895 | −1.113485639 | 6.66E−08 | 4.45E−06 | 100.7145473 | 103.9209125 | 94.58334024 |
| ENSMUSG00000039103 | −1.109845105 | 0.002277587 | 0.018174053 | 37.89260194 | 21.3669166 | 26.63026084 |
| ENSMUSG00000039304 | 1.09876453 | 1.10E−06 | 4.76E−05 | 197.4403996 | 176.7626737 | 181.8204016 |
| ENSMUSG00000039533 | 1.597388363 | 4.59E−08 | 3.26E−06 | 367.9571083 | 378.7771578 | 293.8511541 |
| ENSMUSG00000039601 | 1.185885146 | 6.77E−14 | 1.97E−11 | 707.9933521 | 644.8923918 | 732.7913156 |
| ENSMUSG00000039704 | 1.942291849 | 0.00175377 | 0.014994461 | 267.2425611 | 391.4030631 | 463.7338526 |
| ENSMUSG00000039741 | 1.087450189 | 5.12E−08 | 3.58E−06 | 264.2510399 | 270.0001279 | 292.0145844 |
| ENSMUSG00000039853 | 1.374596414 | 3.52E−09 | 3.36E−07 | 477.6462192 | 438.0217902 | 491.2823983 |
| ENSMUSG00000039981 | 1.024216498 | 5.34E−05 | 0.001113305 | 116.669327 | 128.2014996 | 147.8438619 |
| ENSMUSG00000040093 | 1.487860932 | 2.33E−07 | 1.27E−05 | 234.3358278 | 188.4173554 | 357.2128093 |
| ENSMUSG00000040128 | −1.050165381 | 1.95E−11 | 3.16E−09 | 1378.094102 | 1310.180477 | 1381.100424 |
| ENSMUSG00000040152 | −1.254644006 | 0.001007039 | 0.009915514 | 35.89825447 | 19.42446963 | 35.81310941 |
| ENSMUSG00000040435 | −1.205190782 | 4.71E−06 | 0.000155637 | 119.6608482 | 131.11517 | 127.6415951 |
| ENSMUSG00000040584 | 1.185136822 | 2.65E−06 | 9.97E−05 | 302.1436418 | 375.8634874 | 406.8001915 |
| ENSMUSG00000040855 | 1.071792937 | 4.60E−07 | 2.25E−05 | 584.3438089 | 647.8060622 | 645.5542543 |
| ENSMUSG00000040891 | −2.088765162 | 3.05E−41 | 2.08E−37 | 406.846884 | 431.2232258 | 360.8859487 |
| ENSMUSG00000041134 | 1.050155799 | 0.006573499 | 0.03826156 | 41.88129688 | 57.30218542 | 84.48220682 |
| ENSMUSG00000041372 | 2.023614953 | 1.33E−05 | 0.000359411 | 51.85303424 | 49.53239756 | 28.46683056 |
| ENSMUSG00000041695 | −1.532466883 | 9.09E−05 | 0.001683702 | 33.903907 | 18.45324615 | 23.87540627 |
| ENSMUSG00000041702 | 1.159708962 | 1.79E−05 | 0.000451095 | 125.6438906 | 178.7051206 | 179.0655471 |
| ENSMUSG00000041920 | −1.612549242 | 5.78E−11 | 8.29E−09 | 130.6297593 | 116.5468178 | 162.5364196 |
| ENSMUSG00000041930 | −2.069670842 | 7.78E−10 | 8.92E−08 | 29.91521206 | 25.25181052 | 29.38511541 |
| ENSMUSG00000041945 | 2.034436558 | 0.000645414 | 0.007184911 | 35.89825447 | 23.30936356 | 20.20226685 |
| ENSMUSG00000042010 | 1.335313237 | 1.72E−08 | 1.42E−06 | 9652.641758 | 5265.973717 | 7079.05796 |
| ENSMUSG00000042115 | −1.249312452 | 6.34E−07 | 3.01E−05 | 57.83607665 | 47.5899506 | 51.42395197 |
| ENSMUSG00000042246 | −1.458803387 | 0.000333225 | 0.004503661 | 16.9519535 | 19.42446963 | 23.87540627 |
| ENSMUSG00000042333 | 1.17659167 | 0.000212792 | 0.003210009 | 83.76259377 | 76.72665505 | 64.27993997 |
| ENSMUSG00000042354 | −1.004338102 | 0.000195353 | 0.003017019 | 312.1153791 | 284.5684801 | 269.057463 |
| ENSMUSG00000042379 | −1.128744357 | 0.001182685 | 0.011115132 | 63.81911906 | 39.82016275 | 89.99191596 |
| ENSMUSG00000042444 | 1.165704626 | 4.84E−10 | 5.85E−08 | 592.3211988 | 520.5757862 | 674.0210848 |
| ENSMUSG00000042510 | −1.154445577 | 0.002024484 | 0.016651324 | 28.91803832 | 29.13670445 | 26.63026084 |
| ENSMUSG00000042607 | −1.584091426 | 0.001074406 | 0.010435667 | 12.96325856 | 6.798564371 | 14.69255771 |
| ENSMUSG00000042622 | −1.343729967 | 0.000106867 | 0.001912519 | 29.91521206 | 22.33814008 | 31.22168513 |
| ENSMUSG00000042680 | 1.086900564 | 0.000262276 | 0.00374505 | 262.2566924 | 328.2735368 | 358.1310941 |
| ENSMUSG00000042743 | 1.143476031 | 0.005459928 | 0.033421057 | 39.88694941 | 35.93526882 | 50.50566712 |
| ENSMUSG00000042745 | −1.243661764 | 2.94E−08 | 2.25E−06 | 425.793185 | 215.6116129 | 317.7265604 |
| ENSMUSG00000043165 | −1.65740279 | 0.002445867 | 0.019147761 | 6.980216147 | 13.59712874 | 9.182848567 |
| ENSMUSG00000043421 | −1.515224307 | 3.07E−05 | 0.000711553 | 31.90955953 | 28.16548097 | 33.9765397 |
| ENSMUSG00000043639 | 1.042566076 | 0.00209562 | 0.017092091 | 83.76259377 | 70.89931416 | 87.23706139 |
| ENSMUSG00000043681 | −1.124534949 | 2.43E−09 | 2.42E−07 | 201.4290945 | 238.9209765 | 202.0226685 |
| ENSMUSG00000044042 | 1.154056163 | 3.06E−05 | 0.000711225 | 258.2679974 | 274.8562453 | 353.5396698 |
| ENSMUSG00000044186 | 1.148916053 | 0.009243418 | 0.048746497 | 35.89825447 | 33.99282186 | 30.30340027 |
| ENSMUSG00000044339 | −1.202694639 | 0.000240284 | 0.0035008 | 44.87281809 | 27.19425749 | 28.46683056 |
| ENSMUSG00000044349 | 1.020247724 | 0.000864482 | 0.008883909 | 170.5167087 | 218.5252834 | 182.7386865 |
| ENSMUSG00000044359 | 1.419594491 | 2.01E−07 | 1.12E−05 | 221.3725692 | 303.9929497 | 263.5477539 |
| ENSMUSG00000044676 | −1.235532178 | 3.06E−06 | 0.000111153 | 173.5082299 | 206.8706016 | 146.0072922 |
| ENSMUSG00000044749 | 1.260739468 | 0.003720434 | 0.025546608 | 2581.682801 | 3424.533996 | 3631.816608 |
| ENSMUSG00000044948 | −1.380324526 | 9.28E−05 | 0.001711935 | 28.91803832 | 18.45324615 | 23.87540627 |
| ENSMUSG00000045045 | 1.563883127 | 9.68E−08 | 6.14E−06 | 106.6975897 | 117.5180413 | 171.7192682 |
| ENSMUSG00000045294 | 1.216661032 | 2.49E−17 | 1.21E−14 | 16820.32657 | 15644.46784 | 18334.47545 |
| ENSMUSG00000045348 | −1.535176629 | 0.002140436 | 0.017319005 | 17.94912724 | 8.741011335 | 9.182848567 |
| ENSMUSG00000045382 | −1.705364762 | 8.30E−05 | 0.001575106 | 17.94912724 | 15.53957571 | 23.87540627 |
| ENSMUSG00000045411 | −1.265704961 | 6.98E−06 | 0.000215157 | 217.3838743 | 180.6475676 | 227.7346445 |
| ENSMUSG00000045776 | 1.435732199 | 4.30E−15 | 1.59E−12 | 1029.083295 | 1166.439401 | 1214.890865 |
| ENSMUSG00000045875 | 1.205673082 | 0.000172777 | 0.002749319 | 91.73998365 | 131.11517 | 117.5404617 |
| ENSMUSG00000046541 | 1.523461987 | 5.84E−06 | 0.000185032 | 69.80216147 | 95.1799012 | 84.48220682 |
| ENSMUSG00000046721 | −1.016110295 | 0.006334733 | 0.037187581 | 17.94912724 | 26.223034 | 22.95712142 |
| ENSMUSG00000046908 | −1.354651164 | 0.005396039 | 0.033236578 | 15.95477976 | 8.741011335 | 10.10113342 |
| ENSMUSG00000047496 | 1.093888518 | 1.32E−05 | 0.00035939 | 433.7705749 | 520.5757862 | 451.7961495 |
| ENSMUSG00000047649 | −1.227032883 | 5.45E−06 | 0.000175137 | 72.79368268 | 67.01442023 | 64.27993997 |
| ENSMUSG00000047875 | 1.019701978 | 0.000156296 | 0.002567963 | 101.711721 | 104.892136 | 85.40049167 |
| ENSMUSG00000048191 | −1.584188448 | 0.000509157 | 0.006122909 | 10.96891109 | 18.45324615 | 11.01941828 |
| ENSMUSG00000048644 | −2.138371107 | 3.35E−05 | 0.00076585 | 5.983042412 | 8.741011335 | 8.26456371 |
| ENSMUSG00000048856 | −1.529839383 | 8.95E−23 | 8.72E−20 | 3855.073661 | 3458.526818 | 4005.558545 |
| ENSMUSG00000049044 | −1.523296713 | 7.27E−17 | 3.42E−14 | 783.7785559 | 1199.461 | 1266.314817 |
| ENSMUSG00000049313 | 1.020089742 | 3.99E−06 | 0.000136353 | 328.0701589 | 236.9785295 | 312.2168513 |
| ENSMUSG00000049580 | 1.860685334 | 2.05E−30 | 5.59E−27 | 548.4455544 | 407.9138623 | 479.3446952 |
| ENSMUSG00000049791 | 1.116973741 | 1.01E−06 | 4.43E−05 | 636.1968431 | 678.8852137 | 759.4215765 |
| ENSMUSG00000049950 | −1.164244145 | 0.007174451 | 0.040867998 | 18.94630097 | 37.87771578 | 21.1205517 |
| ENSMUSG00000050390 | 1.148547066 | 4.80E−15 | 1.72E−12 | 1574.537328 | 1571.439593 | 1292.026793 |
| ENSMUSG00000050503 | −1.747508686 | 0.001612805 | 0.014098606 | 6.980216147 | 9.712234816 | 7.346278854 |
| ENSMUSG00000050663 | 1.366962359 | 0.003061462 | 0.022321797 | 20.94064844 | 51.47484453 | 44.07767312 |
| ENSMUSG00000050737 | −1.262831751 | 0.008424555 | 0.045643887 | 26.92369085 | 13.59712874 | 44.99595798 |
| ENSMUSG00000050914 | −2.867341906 | 0.000724516 | 0.007829023 | 13.96043229 | 23.30936356 | 14.69255771 |
| ENSMUSG00000051149 | 1.736018564 | 0.001879809 | 0.015833824 | 31.90955953 | 33.02159838 | 39.48624884 |
| ENSMUSG00000051339 | 1.051356464 | 2.17E−16 | 9.53E−14 | 1923.548135 | 1976.439785 | 1850.343986 |
| ENSMUSG00000051452 | 1.412708477 | 1.55E−10 | 2.11E−08 | 276.2171247 | 240.8634234 | 283.7500207 |
| ENSMUSG00000051674 | 1.054513908 | 9.53E−09 | 8.39E−07 | 515.5388212 | 743.9571869 | 608.82286 |
| ENSMUSG00000051998 | −1.5364341 | 0.002646663 | 0.020230437 | 9.971737353 | 19.42446963 | 6.427993997 |
| ENSMUSG00000052085 | 1.272820714 | 3.30E−11 | 4.99E−09 | 423.7988375 | 546.7988202 | 446.2864404 |
| ENSMUSG00000052595 | 1.233431784 | 0.003777269 | 0.025781088 | 1666.277312 | 2467.878867 | 2847.601341 |
| ENSMUSG00000052656 | 1.063492715 | 4.09E−12 | 7.63E−10 | 4427.451385 | 3448.814583 | 3382.043127 |
| ENSMUSG00000052684 | −1.26752533 | 1.09E−07 | 6.78E−06 | 161.5421451 | 161.223098 | 181.8204016 |
| ENSMUSG00000052713 | 1.631079765 | 2.29E−06 | 8.94E−05 | 74.78803015 | 84.4964429 | 102.8479039 |
| ENSMUSG00000052837 | −1.91589408 | 1.98E−18 | 1.08E−15 | 205.4177895 | 302.0505028 | 276.4037419 |
| ENSMUSG00000053560 | −1.145431158 | 4.41E−10 | 5.44E−08 | 324.081464 | 268.0576809 | 242.4272022 |
| ENSMUSG00000053964 | −1.860738134 | 2.78E−14 | 8.62E−12 | 100.7145473 | 119.4604882 | 153.3535711 |
| ENSMUSG00000053977 | −1.428502316 | 0.007562752 | 0.042337131 | 8.974563618 | 28.16548097 | 7.346278854 |
| ENSMUSG00000054008 | 1.359856014 | 0.005424595 | 0.033352211 | 3190.955953 | 3047.699285 | 3392.144261 |
| ENSMUSG00000054150 | 1.583267643 | 1.23E−07 | 7.44E−06 | 98.72019979 | 105.8633595 | 78.97249768 |
| ENSMUSG00000054422 | −1.006699851 | 5.17E−07 | 2.51E−05 | 21378.40771 | 26089.97639 | 27702.81756 |
| ENSMUSG00000054453 | 1.090734081 | 0.000462815 | 0.005711685 | 93.73433112 | 87.41011335 | 126.7233102 |
| ENSMUSG00000054659 | 1.244126007 | 0.002259781 | 0.018053098 | 79.77389882 | 73.8129846 | 100.0930494 |
| ENSMUSG00000054932 | 1.219377704 | 9.42E−05 | 0.001724207 | 87.75128871 | 202.0144842 | 233.2443536 |
| ENSMUSG00000055148 | −1.210391107 | 1.04E−07 | 6.56E−06 | 210.4036581 | 153.4533101 | 137.7427285 |
| ENSMUSG00000055254 | 1.702350158 | 1.36E−13 | 3.56E−11 | 1260.427601 | 1100.396205 | 2139.603716 |
| ENSMUSG00000055491 | −1.429157403 | 5.36E−08 | 3.69E−06 | 191.4573572 | 205.8993781 | 144.1707225 |
| ENSMUSG00000055660 | 1.21539125 | 9.47E−05 | 0.001725945 | 86.75411497 | 97.12234816 | 112.0307525 |
| ENSMUSG00000055692 | −1.507668302 | 6.91E−05 | 0.001367589 | 23.93216965 | 24.28058704 | 19.28398199 |
| ENSMUSG00000055980 | 1.007478258 | 8.74E−05 | 0.001638705 | 336.0475488 | 377.8059344 | 381.0882155 |
| ENSMUSG00000056054 | −4.00774487 | 1.39E−20 | 1.11E−17 | 6.980216147 | 6.798564371 | 4.591424283 |
| ENSMUSG00000056071 | −3.639131514 | 1.15E−24 | 1.74E−21 | 15.95477976 | 8.741011335 | 12.85598799 |
| ENSMUSG00000056091 | −1.031452251 | 1.62E−09 | 1.68E−07 | 1990.358776 | 1481.115809 | 2190.109383 |
| ENSMUSG00000056148 | 1.3596028 | 0.000119043 | 0.002094688 | 75.78520388 | 108.7770299 | 71.62621882 |
| ENSMUSG00000056313 | −1.726808914 | 2.85E−11 | 4.37E−09 | 435.7649223 | 244.7483174 | 415.0647552 |
| ENSMUSG00000057342 | 1.09304789 | 8.77E−15 | 2.92E−12 | 1269.402165 | 1384.964685 | 1236.011417 |
| ENSMUSG00000057604 | −1.272122676 | 0.008168023 | 0.044709526 | 10.96891109 | 10.6834583 | 11.93770314 |
| ENSMUSG00000057722 | −1.840565518 | 1.78E−07 | 1.02E−05 | 45.86999182 | 35.93526882 | 110.1941828 |
| ENSMUSG00000057969 | 1.066228289 | 0.000219235 | 0.003267437 | 95.72867859 | 74.78420809 | 99.17476452 |
| ENSMUSG00000058207 | −1.338541988 | 1.08E−13 | 2.88E−11 | 23432.58561 | 23413.28447 | 28330.9244 |
| ENSMUSG00000058503 | −1.136818503 | 6.67E−07 | 3.10E−05 | 130.6297593 | 164.1367684 | 153.3535711 |
| ENSMUSG00000058793 | 1.056167061 | 4.57E−10 | 5.57E−08 | 3494.096768 | 3071.008649 | 3164.409616 |
| ENSMUSG00000058794 | 1.61977926 | 2.23E−06 | 8.73E−05 | 144.5901916 | 102.9496891 | 144.1707225 |
| ENSMUSG00000058921 | 1.372756574 | 4.51E−07 | 2.22E−05 | 911.4167941 | 1126.619239 | 1263.559963 |
| ENSMUSG00000059149 | 1.084964814 | 0.00015449 | 0.002547498 | 343.0277649 | 296.2231619 | 348.9482455 |
| ENSMUSG00000059824 | 4.510710868 | 5.51E−13 | 1.32E−10 | 248.2962601 | 730.3600582 | 248.8551962 |
| ENSMUSG00000060429 | 1.717674566 | 1.22E−12 | 2.68E−10 | 734.9170429 | 831.3673003 | 944.9151175 |
| ENSMUSG00000061175 | 1.152852085 | 3.29E−14 | 9.98E−12 | 1100.879804 | 976.079599 | 989.9110755 |
| ENSMUSG00000061292 | 1.112980568 | 1.04E−05 | 0.000296719 | 1260.427601 | 1520.935972 | 1350.797024 |
| ENSMUSG00000061436 | 1.042732241 | 2.40E−05 | 0.000578522 | 326.0758114 | 431.2232258 | 663.9199514 |
| ENSMUSG00000061536 | 1.045389319 | 1.00E−05 | 0.000287463 | 219.3782218 | 260.2878931 | 199.2678139 |
| ENSMUSG00000061825 | 1.826944112 | 9.41E−14 | 2.63E−11 | 718.9622632 | 690.5398954 | 678.6125091 |
| ENSMUSG00000062901 | 1.319446488 | 6.22E−09 | 5.70E−07 | 788.7644246 | 893.5256031 | 992.6659301 |
| ENSMUSG00000063535 | 1.201850675 | 0.000151738 | 0.002508185 | 62.82194532 | 71.87053764 | 70.70793397 |
| ENSMUSG00000063704 | −1.127471271 | 2.47E−07 | 1.32E−05 | 294.1662519 | 311.7627376 | 224.061505 |
| ENSMUSG00000063929 | −1.198836957 | 5.83E−06 | 0.000185032 | 50.8558605 | 105.8633595 | 82.6456371 |
| ENSMUSG00000065126 | −1.713055356 | 1.38E−07 | 8.18E−06 | 29.91521206 | 48.56117408 | 40.40453369 |
| ENSMUSG00000065147 | −1.294541943 | 0.007255788 | 0.041125179 | 14.95760603 | 12.62590526 | 15.61084256 |
| ENSMUSG00000065952 | 1.57267976 | 1.62E−10 | 2.19E−08 | 199.4347471 | 157.338204 | 188.2483956 |
| ENSMUSG00000066456 | 1.032022635 | 0.005405213 | 0.033278057 | 76.78237762 | 64.10074979 | 150.5987165 |
| ENSMUSG00000066477 | −1.128543552 | 2.29E−05 | 0.000561201 | 57.83607665 | 44.67628015 | 49.58738226 |
| ENSMUSG00000066687 | −2.591322343 | 5.98E−23 | 6.28E−20 | 139.6043229 | 122.3741587 | 161.6181348 |
| ENSMUSG00000066944 | 1.544707207 | 0.000412457 | 0.005261624 | 43.87564435 | 49.53239756 | 40.40453369 |
| ENSMUSG00000067149 | −1.843573184 | 0.000282252 | 0.003980428 | 113.6778058 | 169.9641093 | 162.5364196 |
| ENSMUSG00000068463 | −2.382320454 | 0.000472976 | 0.005821273 | 4.985868677 | 3.884893927 | 5.50970914 |
| ENSMUSG00000068742 | 1.190690012 | 3.19E−12 | 6.14E−10 | 611.2674997 | 770.1802209 | 691.4684971 |
| ENSMUSG00000068877 | −1.095647878 | 6.50E−07 | 3.04E−05 | 98.72019979 | 98.09357164 | 136.8244436 |
| ENSMUSG00000069456 | 1.727916701 | 1.48E−26 | 2.89E−23 | 6191.451722 | 5969.139518 | 5731.934075 |
| ENSMUSG00000069804 | −2.067148134 | 1.72E−05 | 0.000437213 | 3.988694941 | 13.59712874 | 13.77427285 |
| ENSMUSG00000070576 | 1.095801322 | 1.06E−07 | 6.63E−06 | 253.2821288 | 274.8562453 | 226.8163596 |
| ENSMUSG00000070583 | 1.188428844 | 0.006226465 | 0.036681868 | 41.88129688 | 47.5899506 | 40.40453369 |
| ENSMUSG00000071076 | 1.243260003 | 3.56E−08 | 2.62E−06 | 1324.24672 | 1340.288405 | 938.4871235 |
| ENSMUSG00000071456 | 1.345823726 | 0.001263422 | 0.011712633 | 45.86999182 | 70.89931416 | 48.6690974 |
| ENSMUSG00000071547 | −1.168738292 | 0.00025901 | 0.003706323 | 44.87281809 | 25.25181052 | 47.75081255 |
| ENSMUSG00000071637 | −1.028910519 | 0.000250761 | 0.0036106 | 107.6947634 | 64.10074979 | 89.99191596 |
| ENSMUSG00000071645 | −1.14015686 | 5.54E−07 | 2.68E−05 | 206.4149632 | 273.8850218 | 188.2483956 |
| ENSMUSG00000072294 | 2.084673623 | 3.53E−08 | 2.62E−06 | 97.72302606 | 148.5971927 | 196.5129593 |
| ENSMUSG00000072571 | 1.01455077 | 0.003907572 | 0.026379983 | 56.83890291 | 47.5899506 | 55.0970914 |
| ENSMUSG00000072664 | 1.275253331 | 1.57E−06 | 6.41E−05 | 951.3037435 | 1401.475484 | 1131.326943 |
| ENSMUSG00000072692 | −1.135811828 | 0.000365333 | 0.004818223 | 34.90108074 | 29.13670445 | 28.46683056 |
| ENSMUSG00000072849 | −2.106422019 | 7.14E−21 | 6.08E−18 | 100.7145473 | 96.15112468 | 102.8479039 |
| ENSMUSG00000072999 | −1.145851135 | 1.86E−07 | 1.05E−05 | 116.669327 | 176.7626737 | 168.0461288 |
| ENSMUSG00000073460 | −1.28577089 | 0.005211876 | 0.032439227 | 18.94630097 | 39.82016275 | 10.10113342 |
| ENSMUSG00000073835 | 2.295482419 | 0.000631649 | 0.007089542 | 332.0588539 | 185.503685 | 325.0728393 |
| ENSMUSG00000074024 | −1.08003527 | 4.28E−05 | 0.000939631 | 43.87564435 | 61.18707934 | 53.26052169 |
| ENSMUSG00000074063 | −2.210318446 | 1.23E−07 | 7.44E−06 | 989.1963454 | 819.7126185 | 1493.131177 |
| ENSMUSG00000074213 | 1.225081814 | 0.003513884 | 0.024460869 | 87.75128871 | 38.84893927 | 37.64967912 |
| ENSMUSG00000074345 | 1.610694413 | 1.86E−06 | 7.43E−05 | 92.73715738 | 95.1799012 | 56.93366111 |
| ENSMUSG00000074375 | 1.123374183 | 1.71E−16 | 7.76E−14 | 1739.070994 | 2059.965005 | 1850.343986 |
| ENSMUSG00000074876 | 1.013446872 | 0.000205306 | 0.003120937 | 101.711721 | 119.4604882 | 117.5404617 |
| ENSMUSG00000075470 | 1.495450545 | 0.008033121 | 0.044154642 | 346.0192861 | 387.5181692 | 422.4110341 |
| ENSMUSG00000075552 | 1.02486804 | 2.18E−07 | 1.20E−05 | 1632.373405 | 1558.813688 | 2640.068963 |
| ENSMUSG00000075590 | −1.36961476 | 1.11E−14 | 3.51E−12 | 556.4229443 | 571.0794072 | 507.8115258 |
| ENSMUSG00000076490 | −1.690670704 | 0.002848332 | 0.021272015 | 9.971737353 | 4.856117408 | 8.26456371 |
| ENSMUSG00000076569 | −1.340585852 | 0.000314538 | 0.004311858 | 26.92369085 | 70.89931416 | 46.83252769 |
| ENSMUSG00000076596 | −3.365323343 | 1.13E−07 | 6.96E−06 | 3.988694941 | 6.798564371 | 9.182848567 |
| ENSMUSG00000076609 | −2.421594655 | 3.22E−06 | 0.000115075 | 484.6264354 | 675.0003197 | 704.3244851 |
| ENSMUSG00000076613 | −1.082440472 | 0.000721125 | 0.007817157 | 31.90955953 | 61.18707934 | 74.38107339 |
| ENSMUSG00000076617 | −1.218011643 | 6.44E−08 | 4.32E−06 | 1755.025774 | 1885.144778 | 1859.526835 |
| ENSMUSG00000076934 | −3.037010136 | 1.44E−09 | 1.52E−07 | 7.977389882 | 2.913670445 | 10.10113342 |
| ENSMUSG00000077148 | 1.938039708 | 8.47E−06 | 0.000252598 | 48.86151303 | 38.84893927 | 69.78964911 |
| ENSMUSG00000078193 | −1.269506646 | 0.004582659 | 0.029506003 | 13.96043229 | 9.712234816 | 21.1205517 |
| ENSMUSG00000078234 | 1.031251449 | 6.47E−07 | 3.04E−05 | 557.420118 | 503.0937635 | 599.6400114 |
| ENSMUSG00000078650 | −1.017019231 | 3.99E−07 | 2.00E−05 | 5816.514398 | 3628.490927 | 4180.950953 |
| ENSMUSG00000078651 | −2.099102297 | 6.20E−07 | 2.95E−05 | 8.974563618 | 10.6834583 | 16.52912742 |
| ENSMUSG00000078672 | −1.051061583 | 1.66E−05 | 0.000428362 | 108.6919371 | 197.1583668 | 152.4352862 |
| ENSMUSG00000078688 | 1.787709931 | 3.76E−11 | 5.58E−09 | 119.6608482 | 143.7410753 | 173.5558379 |
| ENSMUSG00000078817 | −1.628755428 | 1.37E−11 | 2.31E−09 | 107.6947634 | 79.64032549 | 80.80906739 |
| ENSMUSG00000079017 | −1.001509959 | 0.000541038 | 0.006404635 | 74.78803015 | 60.21585586 | 44.07767312 |
| ENSMUSG00000079036 | −1.378588057 | 0.008915895 | 0.047568881 | 170.5167087 | 124.3166056 | 106.5210434 |
| ENSMUSG00000079065 | −1.153356662 | 4.39E−06 | 0.000146637 | 123.6495432 | 164.1367684 | 125.8050254 |
| ENSMUSG00000079465 | −1.030415111 | 0.006780187 | 0.03914539 | 18.94630097 | 27.19425749 | 22.03883656 |
| ENSMUSG00000079470 | 1.222302248 | 2.09E−11 | 3.33E−09 | 498.5868677 | 463.2736007 | 474.7532709 |
| ENSMUSG00000080059 | −1.546208951 | 0.008392591 | 0.045561211 | 7.977389882 | 9.712234816 | 6.427993997 |
| ENSMUSG00000081344 | −1.007584184 | 0.007335419 | 0.041406148 | 22.93499591 | 19.42446963 | 38.56796398 |
| ENSMUSG00000082065 | 2.441656125 | 0.003600075 | 0.02496215 | 179.4912724 | 141.7986283 | 255.2831902 |
| ENSMUSG00000082173 | 2.591199516 | 2.10E−11 | 3.33E−09 | 150.573234 | 114.6043708 | 224.061505 |
| ENSMUSG00000082586 | 1.935822229 | 3.52E−06 | 0.000123572 | 53.84738171 | 55.35973845 | 47.75081255 |
| ENSMUSG00000082658 | −1.204694651 | 0.004700851 | 0.030195718 | 67.807814 | 23.30936356 | 23.87540627 |
| ENSMUSG00000083327 | 1.031096879 | 0.000937166 | 0.009424872 | 102.7088947 | 96.15112468 | 82.6456371 |
| ENSMUSG00000083621 | −1.163358375 | 0.003315291 | 0.023547198 | 19.94347471 | 19.42446963 | 20.20226685 |
| ENSMUSG00000083716 | −1.168157812 | 0.001666635 | 0.01443041 | 25.92651712 | 25.25181052 | 35.81310941 |
| ENSMUSG00000083813 | −1.588176086 | 8.35E−05 | 0.001580916 | 11.96608482 | 53.41729149 | 22.03883656 |
| ENSMUSG00000083863 | −1.215047324 | 0.000627696 | 0.007061263 | 24.92934338 | 19.42446963 | 27.5485457 |
| ENSMUSG00000083992 | −1.283026783 | 0.003457083 | 0.024176532 | 11.96608482 | 12.62590526 | 20.20226685 |
| ENSMUSG00000084822 | −1.954137616 | 1.52E−07 | 8.88E−06 | 16.9519535 | 25.25181052 | 13.77427285 |
| ENSMUSG00000084883 | 1.839966406 | 9.14E−09 | 8.09E−07 | 157.5534502 | 141.7986283 | 126.7233102 |
| ENSMUSG00000085001 | −2.14213554 | 3.42E−08 | 2.56E−06 | 29.91521206 | 16.51079919 | 21.1205517 |
| ENSMUSG00000085156 | −1.793249778 | 0.00014396 | 0.002408808 | 24.92934338 | 31.07915141 | 16.52912742 |
| ENSMUSG00000085445 | −1.469856228 | 8.55E−08 | 5.52E−06 | 45.86999182 | 46.61872712 | 72.54450368 |
| ENSMUSG00000085834 | −3.692490083 | 4.07E−39 | 1.85E−35 | 50.8558605 | 56.33096193 | 71.62621882 |
| ENSMUSG00000085995 | −1.230019641 | 9.11E−06 | 0.000268289 | 189.4630097 | 167.0504388 | 353.5396698 |
| ENSMUSG00000086140 | 1.216209638 | 0.003436989 | 0.024085413 | 34.90108074 | 60.21585586 | 44.99595798 |
| ENSMUSG00000086446 | 2.158985376 | 0.000113634 | 0.002012508 | 17.94912724 | 6.798564371 | 9.182848567 |
| ENSMUSG00000086529 | 2.155075918 | 4.95E−05 | 0.001053778 | 27.92086459 | 38.84893927 | 44.07767312 |
| ENSMUSG00000086786 | 1.909493382 | 0.004138057 | 0.027366965 | 31.90955953 | 14.56835222 | 33.05825484 |
| ENSMUSG00000086844 | −1.797314635 | 1.20E−05 | 0.000331862 | 10.96891109 | 17.48202267 | 18.36569713 |
| ENSMUSG00000087382 | −1.074896574 | 1.10E−05 | 0.000308734 | 161.5421451 | 285.5397036 | 136.8244436 |
| ENSMUSG00000087445 | −1.629934605 | 4.98E−05 | 0.001058506 | 21.93782218 | 12.62590526 | 15.61084256 |
| ENSMUSG00000087595 | −1.356715257 | 0.000782501 | 0.008272069 | 15.95477976 | 21.3669166 | 17.44741228 |
| ENSMUSG00000087613 | 1.300460925 | 0.002649524 | 0.020230437 | 53.84738171 | 60.21585586 | 82.6456371 |
| ENSMUSG00000087616 | −2.549514093 | 7.92E−06 | 0.000238545 | 14.95760603 | 13.59712874 | 8.26456371 |
| ENSMUSG00000087658 | −1.652423913 | 0.001338341 | 0.012240744 | 12.96325856 | 5.82734089 | 9.182848567 |
| ENSMUSG00000089726 | −1.506918387 | 4.30E−05 | 0.000942053 | 73.79085641 | 59.24463238 | 45.91424283 |
| ENSMUSG00000089943 | 1.766690487 | 7.03E−13 | 1.62E−10 | 3075.2838 | 2604.821378 | 3147.880489 |
| ENSMUSG00000090021 | −1.656392969 | 0.002988368 | 0.021957099 | 5.983042412 | 7.769787853 | 19.28398199 |
| ENSMUSG00000090145 | 1.252091278 | 1.05E−06 | 4.56E−05 | 514.5416474 | 669.1729788 | 606.0680054 |
| ENSMUSG00000090175 | 1.660039728 | 0.006419999 | 0.037574902 | 840.6174589 | 1098.453758 | 415.0647552 |
| ENSMUSG00000090264 | −2.18334921 | 1.38E−07 | 8.18E−06 | 7.977389882 | 12.62590526 | 21.1205517 |
| ENSMUSG00000090369 | 1.713925914 | 3.56E−08 | 2.62E−06 | 82.76542003 | 126.2590526 | 106.5210434 |
| ENSMUSG00000090555 | −2.901973235 | 9.10E−10 | 1.01E−07 | 121.6551957 | 110.7194769 | 103.7661888 |
| ENSMUSG00000090610 | 1.076721434 | 0.00270073 | 0.020438322 | 79.77389882 | 53.41729149 | 47.75081255 |
| ENSMUSG00000090698 | −2.55687976 | 1.21E−08 | 1.04E−06 | 20.94064844 | 19.42446963 | 24.79369113 |
| ENSMUSG00000091021 | −1.527245218 | 0.003008341 | 0.022044462 | 10.96891109 | 16.51079919 | 18.36569713 |
| ENSMUSG00000091509 | −1.002937779 | 0.000805178 | 0.008420412 | 69.80216147 | 64.10074979 | 68.87136425 |
| ENSMUSG00000092075 | −5.716673555 | 4.92E−16 | 1.97E−13 | 2.991521206 | 2.913670445 | 0 |
| ENSMUSG00000094410 | 1.220382244 | 0.001002234 | 0.009882478 | 53.84738171 | 132.0863935 | 116.6221768 |
| ENSMUSG00000095280 | −1.650205335 | 4.03E−05 | 0.000893784 | 58.83325038 | 25.25181052 | 24.79369113 |
| ENSMUSG00000095351 | −3.092804449 | 0.006028299 | 0.035789253 | 6.980216147 | 60.21585586 | 166.2095591 |
| ENSMUSG00000096833 | 3.208484861 | 0.004128622 | 0.02736411 | 10.96891109 | 124.3166056 | 23.87540627 |
| ENSMUSG00000096910 | 1.124675865 | 0.006036 | 0.035819378 | 64.81629279 | 67.01442023 | 72.54450368 |
| ENSMUSG00000096954 | 1.206343759 | 0.003120612 | 0.022529545 | 33.903907 | 87.41011335 | 81.72735225 |
| ENSMUSG00000097124 | 2.16107918 | 3.03E−13 | 7.50E−11 | 133.6212805 | 103.9209125 | 130.3964497 |
| ENSMUSG00000097221 | 1.550011559 | 0.000809985 | 0.008457711 | 46.86716556 | 44.67628015 | 42.24110341 |
| ENSMUSG00000097312 | −1.433041605 | 0.000236458 | 0.003456142 | 49.85868677 | 34.96404534 | 33.9765397 |
| ENSMUSG00000097536 | 1.179506676 | 0.003789911 | 0.025815692 | 36.89542821 | 55.35973845 | 40.40453369 |
| ENSMUSG00000097615 | −1.278009058 | 7.24E−05 | 0.001413729 | 33.903907 | 25.25181052 | 25.71197599 |
| ENSMUSG00000097660 | −2.932543755 | 3.83E−06 | 0.000131806 | 4.985868677 | 4.856117408 | 6.427993997 |
| ENSMUSG00000097691 | 1.274551418 | 4.53E−08 | 3.23E−06 | 235.3330015 | 259.3166696 | 175.3924076 |
| ENSMUSG00000097743 | −1.078551643 | 0.000185309 | 0.002900208 | 55.84172918 | 55.35973845 | 50.50566712 |
| ENSMUSG00000097908 | 1.156232879 | 5.54E−05 | 0.001144881 | 154.561929 | 95.1799012 | 98.25647967 |
| ENSMUSG00000097971 | −1.77227667 | 3.92E−13 | 9.56E−11 | 2017.282467 | 2103.670061 | 1313.147345 |
| ENSMUSG00000097994 | −1.156597047 | 0.008206265 | 0.04482173 | 14.95760603 | 25.25181052 | 11.93770314 |
| ENSMUSG00000098041 | −1.469996023 | 0.000332181 | 0.004498467 | 22.93499591 | 20.39569311 | 17.44741228 |
| ENSMUSG00000098661 | −1.627002493 | 0.000277688 | 0.003922434 | 19.94347471 | 6.798564371 | 14.69255771 |
| ENSMUSG00000098814 | −4.705165719 | 0.001128634 | 0.010794205 | 2.991521206 | 1.942446963 | 1.836569713 |
| ENSMUSG00000098882 | 1.905068974 | 0.000139968 | 0.002360908 | 128.6354119 | 27.19425749 | 93.66505538 |
| ENSMUSG00000099568 | 2.251364762 | 0.000320113 | 0.004365382 | 1.994347471 | 4.856117408 | 19.28398199 |
| ENSMUSG00000099858 | 1.088333588 | 4.80E−05 | 0.001024996 | 154.561929 | 202.0144842 | 168.0461288 |
| ENSMUSG00000100094 | 1.758512646 | 5.46E−23 | 6.28E−20 | 3175.001173 | 2305.684545 | 3195.631301 |
| ENSMUSG00000100468 | 1.995883006 | 0.000292618 | 0.004105376 | 17.94912724 | 36.9064923 | 34.89482455 |
| ENSMUSG00000101939 | −1.264629649 | 0.000784437 | 0.008279693 | 14.95760603 | 28.16548097 | 21.1205517 |
| ENSMUSG00000102275 | −1.950030022 | 0.000225327 | 0.003321924 | 4.985868677 | 13.59712874 | 14.69255771 |
| ENSMUSG00000102577 | −1.649520189 | 1.70E−08 | 1.42E−06 | 40.88412315 | 64.10074979 | 82.6456371 |
| ENSMUSG00000102719 | −1.730894623 | 0.006769906 | 0.039102589 | 12.96325856 | 5.82734089 | 4.591424283 |
| ENSMUSG00000102869 | 1.362067771 | 5.85E−06 | 0.000185032 | 292.1719044 | 335.0721012 | 361.8042335 |
| ENSMUSG00000102882 | 2.19227753 | 3.68E−07 | 1.87E−05 | 88.74846244 | 63.12952631 | 216.7152262 |
| ENSMUSG00000102918 | 1.26420677 | 0.002814898 | 0.021114835 | 30.91238579 | 47.5899506 | 66.11650968 |
| ENSMUSG00000103285 | −1.692405475 | 0.00269655 | 0.020438322 | 9.971737353 | 5.82734089 | 8.26456371 |
| ENSMUSG00000103546 | −1.97976669 | 0.000414043 | 0.005267075 | 6.980216147 | 6.798564371 | 6.427993997 |
| ENSMUSG00000104030 | −3.028455317 | 2.18E−07 | 1.20E−05 | 3.988694941 | 4.856117408 | 5.50970914 |
| ENSMUSG00000104388 | −2.61061807 | 5.15E−05 | 0.001078975 | 6.980216147 | 1.942446963 | 6.427993997 |
| ENSMUSG00000104399 | −1.177892321 | 0.00272173 | 0.020574411 | 23.93216965 | 29.13670445 | 18.36569713 |
| ENSMUSG00000104445 | 1.012908858 | 0.003001159 | 0.022015494 | 68.80498774 | 51.47484453 | 89.99191596 |
| ENSMUSG00000104973 | −2.007621104 | 9.36E−05 | 0.001719883 | 6.980216147 | 13.59712874 | 4.591424283 |
| ENSMUSG00000105161 | −1.279752452 | 0.00691512 | 0.039789659 | 21.93782218 | 8.741011335 | 13.77427285 |
| ENSMUSG00000105434 | −2.02241612 | 0.000742647 | 0.007975668 | 5.983042412 | 7.769787853 | 6.427993997 |
| ENSMUSG00000105547 | −2.707072316 | 4.65E−06 | 0.000153843 | 5.983042412 | 2.913670445 | 8.26456371 |
| ENSMUSG00000105556 | 1.504518371 | 0.00309422 | 0.022420764 | 21.93782218 | 29.13670445 | 33.05825484 |
| ENSMUSG00000105703 | −1.801332207 | 3.38E−12 | 6.40E−10 | 242.3132177 | 122.3741587 | 160.6998499 |
| ENSMUSG00000105881 | 1.259909682 | 7.19E−05 | 0.001410701 | 247.2990864 | 245.7195409 | 252.5283356 |
| ENSMUSG00000105906 | −2.671616891 | 0.003078263 | 0.022366209 | 6.980216147 | 20.39569311 | 14.69255771 |
| ENSMUSG00000106030 | 1.145150269 | 5.54E−05 | 0.001144881 | 109.6891109 | 147.6259692 | 198.349529 |
| ENSMUSG00000106664 | −1.606191029 | 0.000152255 | 0.002513686 | 9.971737353 | 17.48202267 | 19.28398199 |
| ENSMUSG00000106705 | −2.05446715 | 2.41E−09 | 2.41E−07 | 18.94630097 | 32.05037489 | 31.22168513 |
| ENSMUSG00000106706 | −1.806072384 | 0.002749571 | 0.020715964 | 9.971737353 | 8.741011335 | 4.591424283 |
| ENSMUSG00000106943 | −1.230168101 | 0.009404129 | 0.049324655 | 9.971737353 | 13.59712874 | 13.77427285 |
| ENSMUSG00000107168 | −2.535260536 | 5.76E−05 | 0.00118059 | 1.994347471 | 9.712234816 | 5.50970914 |
| ENSMUSG00000107225 | 3.314781204 | 7.31E−09 | 6.61E−07 | 3.988694941 | 1.942446963 | 6.427993997 |
| ENSMUSG00000107304 | −1.772473058 | 0.001145745 | 0.010886341 | 7.977389882 | 10.6834583 | 11.93770314 |
| ENSMUSG00000107390 | −1.580957843 | 0.000625578 | 0.007061263 | 12.96325856 | 23.30936356 | 11.93770314 |
| ENSMUSG00000107624 | −2.865902972 | 2.57E−10 | 3.34E−08 | 8.974563618 | 13.59712874 | 11.93770314 |
| ENSMUSG00000108368 | −1.782724603 | 5.57E−05 | 0.001147878 | 20.94064844 | 11.65468178 | 7.346278854 |
| ENSMUSG00000108633 | −1.682864395 | 0.001365295 | 0.012400579 | 8.974563618 | 12.62590526 | 9.182848567 |
| ENSMUSG00000108820 | −1.905612833 | 0.000888445 | 0.00904159 | 6.980216147 | 3.884893927 | 8.26456371 |
| ENSMUSG00000108825 | 1.256782178 | 0.000111922 | 0.001987343 | 104.7032422 | 117.5180413 | 191.0032502 |
| ENSMUSG00000109089 | −1.779914445 | 1.06E−06 | 4.61E−05 | 42.87847062 | 34.96404534 | 96.41990995 |
| ENSMUSG00000109115 | 2.077699358 | 0.000502824 | 0.006083704 | 26.92369085 | 27.19425749 | 19.28398199 |
| ENSMUSG00000109157 | −1.885864825 | 0.000870552 | 0.00892677 | 4.985868677 | 6.798564371 | 8.26456371 |
| ENSMUSG00000109262 | 1.329001341 | 0.002840836 | 0.0212393 | 48.86151303 | 45.64750364 | 24.79369113 |
| ENSMUSG00000109291 | −1.949878064 | 0.001232984 | 0.01148511 | 1.994347471 | 9.712234816 | 9.182848567 |
| ENSMUSG00000109536 | −1.039899156 | 0.001827878 | 0.015463254 | 63.81911906 | 37.87771578 | 29.38511541 |
| ENSMUSG00000109555 | −1.255499819 | 0.007385438 | 0.041635066 | 10.96891109 | 14.56835222 | 15.61084256 |
| ENSMUSG00000109807 | −2.757236067 | 2.47E−09 | 2.44E−07 | 11.96608482 | 5.82734089 | 12.85598799 |
| ENSMUSG00000109836 | 1.535086391 | 0.000730915 | 0.007885666 | 46.86716556 | 68.9568672 | 89.0736311 |
| ENSMUSG00000109841 | 1.421032277 | 0.000627493 | 0.007061263 | 46.86716556 | 57.30218542 | 33.05825484 |
| ENSMUSG00000110588 | −5.501119532 | 2.73E−06 | 0.000102051 | 31.90955953 | 14.56835222 | 3.673139427 |
| ENSMUSG00000110613 | 1.168711997 | 0.002147567 | 0.017360028 | 64.81629279 | 58.2734089 | 38.56796398 |
| ENSMUSG00000110702 | −1.389252147 | 0.004080864 | 0.027194141 | 18.94630097 | 11.65468178 | 8.26456371 |
| ENSMUSG00000110755 | 1.852337964 | 4.42E−06 | 0.000147317 | 194.4488784 | 92.26623075 | 176.3106925 |
| ENSMUSG00000111282 | −1.321989153 | 0.002140143 | 0.017319005 | 21.93782218 | 18.45324615 | 30.30340027 |
| ENSMUSG00000111312 | 1.254957582 | 0.001977069 | 0.016387107 | 45.86999182 | 33.99282186 | 56.93366111 |
| ENSMUSG00000111631 | −1.948317608 | 1.41E−05 | 0.000376102 | 11.96608482 | 15.53957571 | 11.01941828 |
| ENSMUSG00000111709 | 2.018073354 | 0.00211263 | 0.017179449 | 31.90955953 | 69.92809068 | 7.346278854 |
| ENSMUSG00000111774 | 2.335615682 | 0.000501244 | 0.006070575 | 17.94912724 | 15.53957571 | 39.48624884 |

| ID | agedliverpbs1 | agedliverpbs2 | agedliverpbs3 | Gene.name |
|---|---|---|---|---|
| ENSMUSG00000000204 | 61.79909158 | 46.97322185 | 22.5130033 | Slfn4 |
| ENSMUSG00000000317 | 92.69863737 | 140.9196656 | 205.5535084 | Bcl6b |
| ENSMUSG00000000686 | 171.5457542 | 161.796653 | 294.6266953 | Abhd15 |
| ENSMUSG00000001227 | 171.5457542 | 203.550628 | 184.9981575 | Sema6b |
| ENSMUSG00000001403 | 51.14407579 | 37.57857748 | 17.61887214 | Ube2c |
| ENSMUSG00000001983 | 128.9256911 | 122.1303768 | 219.2570756 | Taco1 |
| ENSMUSG00000002233 | 330.3054895 | 288.1024274 | 298.5420002 | Rhoc |
| ENSMUSG00000002250 | 627.58043 | 657.6251059 | 647.9829644 | Ppard |
| ENSMUSG00000002289 | 1865.693265 | 2130.496573 | 2428.467877 | Angptl4 |
| ENSMUSG00000002831 | 228.0173379 | 574.117156 | 226.1088592 | Plin4 |
| ENSMUSG00000003032 | 155.5632305 | 107.5164856 | 114.5226689 | Klf4 |
| ENSMUSG00000003348 | 13.85152053 | 30.27163186 | 33.28009183 | Mob3a |
| ENSMUSG00000003500 | 174.742259 | 99.16569058 | 90.05201318 | Impdh1 |
| ENSMUSG00000003541 | 105.4846563 | 45.92937248 | 44.04718036 | Ier3 |
| ENSMUSG00000003848 | 522.0957737 | 291.2339755 | 260.3677772 | Nob1 |
| ENSMUSG00000004100 | 1249.833352 | 729.6507128 | 351.3986167 | Ppan |
| ENSMUSG00000004933 | 42.62006316 | 14.61389124 | 19.57652461 | Matk |
| ENSMUSG00000004951 | 237.6068521 | 636.7481184 | 278.9654756 | Hspb1 |
| ENSMUSG00000005148 | 19.17902842 | 27.14008374 | 22.5130033 | Klf5 |
| ENSMUSG00000005547 | 5271.036311 | 4402.956662 | 4447.78639 | Cyp2a5 |
| ENSMUSG00000005580 | 86.3056279 | 93.9464437 | 169.3369378 | Adcy9 |
| ENSMUSG00000006050 | 2542.286767 | 2021.936238 | 1365.462591 | Sra1 |
| ENSMUSG00000006134 | 225.8863347 | 252.6115486 | 375.8692724 | Crkl |
| ENSMUSG00000006517 | 1097.466626 | 579.3364028 | 613.7240464 | Mvd |
| ENSMUSG00000006587 | 10.65501579 | 2.087698749 | 4.894131151 | Snai3 |
| ENSMUSG00000006711 | 210.9693126 | 141.9635149 | 177.1675477 | D130043K22Rik |
| ENSMUSG00000006777 | 8.524012632 | 8.350794996 | 10.76708853 | Krt23 |
| ENSMUSG00000008153 | 96.96064369 | 120.0426781 | 78.30609842 | Clstn3 |
| ENSMUSG00000009013 | 329.2399879 | 396.6627623 | 281.9019543 | Dynll1 |
| ENSMUSG00000009633 | 2971.683904 | 3862.242686 | 1956.673634 | G0s2 |
| ENSMUSG00000014547 | 21.31003158 | 36.53472811 | 46.98365905 | Wdfy2 |
| ENSMUSG00000014609 | 14.91702211 | 14.61389124 | 15.66121968 | Chrne |
| ENSMUSG00000015224 | 318.5849721 | 255.7430968 | 158.5698493 | Cyp2j9 |
| ENSMUSG00000015312 | 121.46718 | 59.49941435 | 63.62370497 | Gadd45b |
| ENSMUSG00000016128 | 199.2487953 | 218.1645193 | 304.4149576 | Stard13 |
| ENSMUSG00000016356 | 90.56763421 | 172.2351468 | 124.3109312 | Col20a1 |
| ENSMUSG00000017737 | 38.35805684 | 32.35933061 | 22.5130033 | Mmp9 |
| ENSMUSG00000017868 | 543.4058053 | 586.6433485 | 498.2225512 | Sgk2 |
| ENSMUSG00000018486 | 3.196504737 | 15.65774062 | 6.851783612 | Wnt9b |
| ENSMUSG00000019082 | 7723.820946 | 7053.290223 | 5201.482588 | Slc25a22 |
| ENSMUSG00000019726 | 179.0042653 | 147.1827618 | 268.1983871 | Lyst |
| ENSMUSG00000019737 | 45.8165679 | 15.65774062 | 18.59769837 | Syne4 |
| ENSMUSG00000019883 | 200.3142968 | 230.6907118 | 405.2340593 | Echdc1 |
| ENSMUSG00000020018 | 268.5063979 | 250.5238499 | 170.3157641 | Snrpf |
| ENSMUSG00000020027 | 2138.461669 | 846.5618427 | 589.2533906 | Socs2 |
| ENSMUSG00000020091 | 393.1700826 | 471.8199173 | 1129.56547 | Eif4ebp2 |
| ENSMUSG00000020122 | 2670.146957 | 1807.947117 | 1885.219319 | Egfr |
| ENSMUSG00000020335 | 24.50653632 | 41.75397498 | 32.3012656 | Zfp354b |
| ENSMUSG00000020429 | 2643.509417 | 2014.629293 | 3936.839098 | Igfbp1 |
| ENSMUSG00000020441 | 303.66795 | 273.4885361 | 168.3581116 | 2310033P09Rik |
| ENSMUSG00000020532 | 2810.793165 | 1739.053058 | 2484.260972 | Acaca |
| ENSMUSG00000020641 | 408.0871047 | 425.8905448 | 452.2177184 | Rsad2 |
| ENSMUSG00000020656 | 71.38860579 | 76.20100434 | 73.41196727 | Grhl1 |
| ENSMUSG00000020681 | 14.91702211 | 21.92083686 | 12.72474099 | Ace |
| ENSMUSG00000020692 | 249.3273695 | 152.4020087 | 104.7344066 | Nle1 |
| ENSMUSG00000020812 | 180.0697668 | 73.06945621 | 57.75074759 | 1810032O08Rik |
| ENSMUSG00000020889 | 443.2486569 | 371.6103773 | 433.62002 | Nr1d1 |
| ENSMUSG00000020917 | 9135.610538 | 6014.660096 | 10059.39717 | Acly |
| ENSMUSG00000020948 | 9.589514211 | 20.87698749 | 44.04718036 | Klhl28 |
| ENSMUSG00000020961 | 25.5720379 | 35.49087873 | 64.6025312 | Ston2 |
| ENSMUSG00000021250 | 204.5763032 | 115.8672806 | 135.0780198 | Fos |
| ENSMUSG00000021260 | 6.393009474 | 11.48234312 | 10.76708853 | Hhipl1 |
| ENSMUSG00000021416 | 404.8906 | 324.6371555 | 254.4948199 | Eci3 |
| ENSMUSG00000021453 | 979.1959511 | 568.8979091 | 678.3265776 | Gadd45g |
| ENSMUSG00000021611 | 220.5588268 | 227.5591636 | 197.7228985 | Tert |
| ENSMUSG00000021670 | 3051.596522 | 1273.496237 | 2047.704474 | Hmgcr |
| ENSMUSG00000021684 | 54.34058053 | 72.02560684 | 49.92013774 | Pde8b |
| ENSMUSG00000021773 | 44.75106632 | 55.32401685 | 29.36478691 | Comtd1 |
| ENSMUSG00000021775 | 635.0389411 | 274.5323855 | 351.3986167 | Nr1d2 |
| ENSMUSG00000021804 | 7.458511053 | 11.48234312 | 20.55535084 | Rgr |
| ENSMUSG00000021958 | 56.47158369 | 91.85874496 | 47.96248528 | Pinx1 |
| ENSMUSG00000022383 | 440.0521521 | 565.766361 | 794.806899 | Ppara |
| ENSMUSG00000022389 | 599.877389 | 502.0915491 | 601.9781316 | Tef |
| ENSMUSG00000022408 | 38.35805684 | 32.35933061 | 21.53417707 | Fam83f |
| ENSMUSG00000022528 | 643.5629537 | 588.7310472 | 500.1802037 | Hes1 |
| ENSMUSG00000022651 | 52.20957737 | 37.57857748 | 32.3012656 | Retnlg |
| ENSMUSG00000022704 | 291.9474326 | 188.9367368 | 134.0991935 | Qtrt2 |
| ENSMUSG00000022853 | 4279.054341 | 3944.706786 | 4891.194673 | Ehhadh |
| ENSMUSG00000022883 | 166.2182463 | 116.9111299 | 103.7555804 | Robo1 |
| ENSMUSG00000022887 | 350.5500195 | 374.7419254 | 819.2775547 | Masp1 |
| ENSMUSG00000022911 | 105.4846563 | 73.06945621 | 75.36961973 | Arl13b |
| ENSMUSG00000023034 | 572.1743479 | 134.6565693 | 149.7604132 | Nr4a1 |
| ENSMUSG00000023044 | 4109.63959 | 5560.585618 | 729O.297763 | Csad |
| ENSMUSG00000023052 | 36.22705368 | 52.19246872 | 20.55535084 | Npff |
| ENSMUSG00000023067 | 1114.514652 | 1009.402345 | 858.4306039 | Cdkn1a |
| ENSMUSG00000023073 | 369.7290479 | 140.9196656 | 157.5910231 | Slc10a2 |
| ENSMUSG00000023341 | 264.2443916 | 178.498243 | 164.4428067 | Mx2 |
| ENSMUSG00000023571 | 53.27507895 | 58.45556497 | 25.44948199 | C1qtnf12 |
| ENSMUSG00000023800 | 160.8907384 | 217.1206699 | 156.6121968 | Tiam2 |
| ENSMUSG00000023905 | 328.1744863 | 207.7260255 | 109.6285378 | Tnfrsf12a |
| ENSMUSG00000023927 | 61.79909158 | 60.54326372 | 33.28009183 | Satb1 |
| ENSMUSG00000023968 | 20.24453 | 38.62242686 | 33.28009183 | Crip3 |
| ENSMUSG00000024118 | 1463.99917 | 1896.674313 | 1329.246021 | Tedc2 |
| ENSMUSG00000024130 | 1006.898992 | 1228.610714 | 2261.088592 | Abca3 |
| ENSMUSG00000024136 | 64.99559632 | 52.19246872 | 34.25891806 | Dnase1l2 |
| ENSMUSG00000024190 | 1247.702349 | 1464.520672 | 1004.275712 | Dusp1 |
| ENSMUSG00000024236 | 484.8032184 | 345.514143 | 575.5498234 | Svil |
| ENSMUSG00000024411 | 14.91702211 | 13.57004187 | 17.61887214 | Aqp4 |
| ENSMUSG00000024440 | 35.16155211 | 70.98175747 | 72.43314104 | Pcdh12 |
| ENSMUSG00000024665 | 3625.901873 | 3398.773563 | 5157.435407 | Fads2 |
| ENSMUSG00000024843 | 3171.998201 | 2942.611387 | 2249.342677 | Chka |
| ENSMUSG00000024887 | 191.7902842 | 179.5420924 | 296.5843478 | Asah2 |
| ENSMUSG00000024924 | 269.5718995 | 262.006193 | 320.0761773 | Vldlr |
| ENSMUSG00000024970 | 90.56763421 | 63.67481184 | 65.58135743 | Al846148 |
| ENSMUSG00000024978 | 1324.418463 | 791.2378259 | 2079.026913 | Gpam |
| ENSMUSG00000025003 | 152.3667258 | 144.0512137 | 186.95581 | Cyp2c39 |
| ENSMUSG00000025006 | 107.6156595 | 110.6480337 | 207.5111608 | Sorbs1 |
| ENSMUSG00000025153 | 35454.56504 | 23677.63536 | 26074.95195 | Fasn |
| ENSMUSG00000025161 | 61.79909158 | 26.09623436 | 32.3012656 | Slc16a3 |
| ENSMUSG00000025240 | 183.2662716 | 163.8843518 | 418.9376266 | Sacm1l |
| ENSMUSG00000025323 | 71.38860579 | 52.19246872 | 60.68722628 | Sp4 |
| ENSMUSG00000025402 | 646.7594584 | 568.8979091 | 502.1378561 | Nab2 |
| ENSMUSG00000025429 | 188.5937795 | 118.9988287 | 253.5159936 | Pstpip2 |
| ENSMUSG00000025450 | 145.9737163 | 103.3410881 | 105.7132329 | Gm9752 |
| ENSMUSG00000025997 | 39.42355842 | 58.45556497 | 22.5130033 | Ikzf2 |
| ENSMUSG00000026020 | 828.9602284 | 520.8808379 | 466.9001118 | Nop58 |
| ENSMUSG00000026249 | 45.8165679 | 122.1303768 | 117.4591476 | Serpine2 |
| ENSMUSG00000026358 | 104.4191547 | 58.45556497 | 96.9037968 | Rgs1 |
| ENSMUSG00000026398 | 615.8599126 | 631.5288716 | 846.6846892 | Nr5a2 |
| ENSMUSG00000026471 | 181.1352684 | 104.3849374 | 126.2685837 | Mr1 |
| ENSMUSG00000026475 | 1039.929541 | 2826.744106 | 2390.293654 | Rgs16 |
| ENSMUSG00000026525 | 43.68556474 | 19.83313812 | 16.64004591 | Opn3 |
| ENSMUSG00000026822 | 304.7334516 | 223.3837661 | 189.8922887 | Lcn2 |
| ENSMUSG00000026826 | 69.25760263 | 25.05238499 | 13.70356722 | Nr4a2 |
| ENSMUSG00000026832 | 101.22265 | 42.79782435 | 62.64487874 | Cytip |
| ENSMUSG00000027360 | 370.7945495 | 319.4179086 | 181.0828526 | Hdc |
| ENSMUSG00000027398 | 98.02614527 | 82.46410059 | 128.2262362 | Il1b |
| ENSMUSG00000027405 | 920.5933642 | 450.9429298 | 352.3774429 | Nop56 |
| ENSMUSG00000027496 | 55.40608211 | 66.80635997 | 66.56018366 | Aurka |
| ENSMUSG00000027513 | 48173.45739 | 29794.5927 | 39823.54518 | Pck1 |
| ENSMUSG00000027605 | 8380.169919 | 3799.611723 | 6353.561061 | Acss2 |
| ENSMUSG00000027762 | 637.1699442 | 555.3278672 | 690.0724923 | Sucnr1 |
| ENSMUSG00000027907 | 207.7728079 | 117.9549793 | 139.9721509 | S100a11 |
| ENSMUSG00000027947 | 713.8860579 | 763.0538928 | 1358.610808 | Il6ra |
| ENSMUSG00000028008 | 20.24453 | 24.00853561 | 36.21657052 | Asic5 |
| ENSMUSG00000028339 | 295.1439374 | 274.5323855 | 289.7325642 | Col15a1 |
| ENSMUSG00000028445 | 91.63313579 | 280.7954817 | 425.7894102 | Enho |
| ENSMUSG00000028630 | 124.6636847 | 101.2533893 | 226.1088592 | Dyrk2 |
| ENSMUSG00000028838 | 74.58511053 | 49.0609206 | 78.30609842 | Extl1 |
| ENSMUSG00000028859 | 152.3667258 | 96.03414245 | 74.3907935 | Csf3r |
| ENSMUSG00000028862 | 77.78161526 | 40.71012561 | 57.75074759 | Map3k6 |
| ENSMUSG00000028864 | 39.42355842 | 69.93790809 | 111.5861902 | Hgf |
| ENSMUSG00000028957 | 62.86459316 | 53.2363181 | 46.00483282 | Per3 |
| ENSMUSG00000028976 | 42.62006316 | 34.44702936 | 45.02600659 | Slc2a5 |
| ENSMUSG00000029086 | 210.9693126 | 165.9720505 | 243.7277313 | Prom1 |
| ENSMUSG00000029135 | 499.7202405 | 272.4446867 | 294.6266953 | Fosl2 |
| ENSMUSG00000029188 | 186.4627763 | 179.5420924 | 182.0616788 | Slc34a2 |
| ENSMUSG00000029195 | 332.4364926 | 209.8137243 | 422.8529315 | Klb |
| ENSMUSG00000029370 | 190.7247826 | 137.7881174 | 169.3369378 | Rassf6 |
| ENSMUSG00000029373 | 25.5720379 | 28.18393311 | 18.59769837 | Pf4 |
| ENSMUSG00000029380 | 615.8599126 | 454.0744779 | 700.8395809 | Cxcl1 |
| ENSMUSG00000029580 | 16369.30076 | 20515.81561 | 13266.0319 | Actb |
| ENSMUSG00000029591 | 177.9387637 | 131.5250212 | 112.5650165 | Ung |
| ENSMUSG00000029656 | 429.3971363 | 614.8272816 | 554.0156463 | C8b |
| ENSMUSG00000030032 | 43.68556474 | 55.32401685 | 35.23774429 | Wdr54 |
| ENSMUSG00000030055 | 75.65061211 | 74.11330559 | 171.2945903 | Rab43 |
| ENSMUSG00000030691 | 189.6592811 | 180.5859418 | 227.0876854 | Fchsd2 |
| ENSMUSG00000030782 | 115.0741705 | 67.85020934 | 70.47548858 | Tgfb1i1 |
| ENSMUSG00000030814 | 814.0432063 | 869.526529 | 459.069502 | Bcl7c |
| ENSMUSG00000030827 | 120.4016784 | 80.37640184 | 48.94131151 | Fgf21 |
| ENSMUSG00000030934 | 5865.586192 | 5683.759844 | 6052.082582 | Oat |
| ENSMUSG00000030968 | 131.0566942 | 132.5688706 | 93.9673181 | Pdilt |
| ENSMUSG00000031010 | 414.4801142 | 361.1718836 | 775.2303744 | Usp9x |
| ENSMUSG00000031271 | 1418.182602 | 1115.874981 | 1330.224847 | Serpina7 |
| ENSMUSG00000031378 | 429.3971363 | 392.4873648 | 441.4506298 | Abcd1 |
| ENSMUSG00000031465 | 50.07857421 | 30.27163186 | 47.96248528 | Angpt2 |
| ENSMUSG00000031762 | 9477.636545 | 4672.2698 | 1607.23267 | Mt2 |
| ENSMUSG00000031765 | 20312.7221 | 10174.39985 | 4243.211708 | Mt1 |
| ENSMUSG00000032009 | 377.187559 | 338.2071973 | 562.8250824 | Sesn3 |
| ENSMUSG00000032064 | 268.5063979 | 302.7163186 | 381.7422298 | Dixdc1 |
| ENSMUSG00000032083 | 163165.5843 | 112907.9676 | 104422.1611 | Apoa1 |
| ENSMUSG00000032091 | 20.24453 | 8.350794996 | 4.894131151 | Tmprss4 |
| ENSMUSG00000032285 | 26.63753947 | 15.65774062 | 44.04718036 | Dnaja4 |
| ENSMUSG00000032417 | 26.63753947 | 43.84167373 | 17.61887214 | Rwdd2a |
| ENSMUSG00000032418 | 6053.11447 | 3739.068459 | 4136.519649 | Me1 |
| ENSMUSG00000032500 | 75.65061211 | 69.93790809 | 123.332105 | Dclk3 |
| ENSMUSG00000032561 | 453.9036726 | 141.9635149 | 317.1396986 | Acpp |
| ENSMUSG00000032702 | 468.8206947 | 629.4411728 | 864.3035613 | Kank1 |
| ENSMUSG00000032724 | 234.4103474 | 176.4105443 | 323.9914822 | Abtb2 |
| ENSMUSG00000032735 | 436.8556474 | 741.1330559 | 606.8722628 | Ablim3 |
| ENSMUSG00000032786 | 7150.581096 | 4035.521682 | 3301.580875 | Alas1 |
| ENSMUSG00000032849 | 168.3492495 | 143.0073643 | 195.7652461 | Abcc4 |
| ENSMUSG00000032860 | 155.5632305 | 134.6565693 | 141.9298034 | P2ry2 |
| ENSMUSG00000032883 | 856.6632695 | 580.3802522 | 723.3525842 | Acsl3 |
| ENSMUSG00000033105 | 1574.811334 | 996.8761526 | 1567.100795 | Lss |
| ENSMUSG00000033594 | 852.4012632 | 820.4656084 | 539.3332529 | Spata2l |
| ENSMUSG00000033624 | 105.4846563 | 98.1218412 | 178.1463739 | Pdpr |
| ENSMUSG00000033792 | 21.31003158 | 26.09623436 | 43.06835413 | Atp7a |
| ENSMUSG00000033855 | 37.29255526 | 42.79782435 | 77.32727219 | Ston1 |
| ENSMUSG00000033967 | 15.98252368 | 19.83313812 | 39.15304921 | Rnf225 |
| ENSMUSG00000034066 | 96.96064369 | 107.5164856 | 184.9981575 | Farp2 |
| ENSMUSG00000034110 | 55.40608211 | 37.57857748 | 55.79309512 | Kctd7 |
| ENSMUSG00000034271 | 135.3187005 | 37.57857748 | 64.6025312 | Jdp2 |
| ENSMUSG00000034755 | 19.17902842 | 36.53472811 | 20.55535084 | Pcdh11x |
| ENSMUSG00000034765 | 166.2182463 | 53.2363181 | 31.32243937 | Dusp5 |
| ENSMUSG00000034853 | 165.1527447 | 137.7881174 | 147.8027608 | Acot11 |
| ENSMUSG00000034926 | 6118.110066 | 6691.074491 | 12774.66113 | Dhcr24 |
| ENSMUSG00000035078 | 94.82964053 | 101.2533893 | 258.4101248 | Mtmr9 |
| ENSMUSG00000035112 | 43.68556474 | 45.92937248 | 36.21657052 | Wnk4 |
| ENSMUSG00000035164 | 61.79909158 | 70.98175747 | 77.32727219 | Zc3h12c |
| ENSMUSG00000035165 | 144.9082147 | 55.32401685 | 91.03083941 | Kcne3 |
| ENSMUSG00000035284 | 293.0129342 | 218.1645193 | 482.5613315 | Vps13c |
| ENSMUSG00000035900 | 147.0392179 | 160.7528037 | 191.8499411 | Gramd4 |
| ENSMUSG00000035933 | 77.78161526 | 81.42025121 | 127.2474099 | Cog5 |
| ENSMUSG00000035948 | 193.9212874 | 154.4897074 | 418.9376266 | Acss3 |
| ENSMUSG00000036062 | 27.70304105 | 29.22778249 | 12.72474099 | Phf24 |
| ENSMUSG00000036120 | 773.5541463 | 626.3096247 | 392.5093183 | Rfxank |
| ENSMUSG00000036611 | 415.5456158 | 267.2254399 | 309.3090888 | Eepc1 |
| ENSMUSG00000037035 | 141.71171 | 149.2704606 | 192.8287674 | Inhbb |
| ENSMUSG00000037071 | 157237.1335 | 63623.66322 | 106805.6029 | Scd1 |
| ENSMUSG00000037095 | 5413.813523 | 8132.630477 | 6142.134595 | Lrg1 |
| ENSMUSG00000037157 | 11.72051737 | 14.61389124 | 24.47065576 | Il22ra1 |
| ENSMUSG00000037336 | 23.44103474 | 42.79782435 | 31.32243937 | Mfsd2b |
| ENSMUSG00000037443 | 3091.020081 | 2288.117829 | 2056.51391 | Cep85 |
| ENSMUSG00000037447 | 99.09164684 | 54.28016747 | 54.81426889 | Arid5a |
| ENSMUSG00000037465 | 592.4188779 | 817.3340602 | 760.5479809 | Klf10 |
| ENSMUSG00000037583 | 304.7334516 | 631.5288716 | 569.676866 | Nr0b2 |
| ENSMUSG00000037709 | 239.7378553 | 289.1462767 | 357.271574 | Fam13a |
| ENSMUSG00000037887 | 80.97812 | 20.87698749 | 30.34361314 | Dusp8 |
| ENSMUSG00000038217 | 1346.793996 | 1375.793476 | 1970.377202 | Tlcd2 |
| ENSMUSG00000038233 | 284.4889216 | 256.7869461 | 354.3350954 | Fam198a |
| ENSMUSG00000038253 | 112.9431674 | 83.50794996 | 44.04718036 | Hoxa5 |
| ENSMUSG00000038370 | 277.0304105 | 627.3534741 | 676.3689251 | Pcp4l1 |
| ENSMUSG00000038415 | 331.3709911 | 448.855231 | 695.9454497 | Foxq1 |
| ENSMUSG00000038418 | 515.7027642 | 342.3825948 | 912.2660466 | Egr1 |
| ENSMUSG00000038473 | 45.8165679 | 35.49087873 | 46.98365905 | Nos1ap |
| ENSMUSG00000038530 | 64.99559632 | 30.27163186 | 63.62370497 | Rgs4 |
| ENSMUSG00000038583 | 67.12659947 | 41.75397498 | 16.64004591 | Pln |
| ENSMUSG00000038587 | 349.4845179 | 176.4105443 | 253.5159936 | Akap12 |
| ENSMUSG00000038751 | 46.88206947 | 44.8855231 | 44.04718036 | Ptk6 |
| ENSMUSG00000038768 | 106.5501579 | 77.24485371 | 93.9673181 | 9130409I23Rik |
| ENSMUSG00000038774 | 194.986789 | 189.9805862 | 328.8856134 | Ascc3 |
| ENSMUSG00000038844 | 169.4147511 | 146.1389124 | 248.6218625 | Kif16b |
| ENSMUSG00000038895 | 230.1483411 | 226.5153143 | 190.8711149 | Zfp653 |
| ENSMUSG00000039103 | 68.19210105 | 74.11330559 | 43.06835413 | Nexn |
| ENSMUSG00000039304 | 105.4846563 | 73.06945621 | 81.24257711 | Tnfsf10 |
| ENSMUSG00000039533 | 167.2837479 | 54.28016747 | 122.3532788 | Mmd2 |
| ENSMUSG00000039601 | 337.7640005 | 298.5409211 | 280.9231281 | Rcan2 |
| ENSMUSG00000039704 | 59.66808842 | 50.10476998 | 182.0616788 | Lmbrd2 |
| ENSMUSG00000039741 | 105.4846563 | 146.1389124 | 137.0356722 | Bahcc1 |
| ENSMUSG00000039853 | 126.7946879 | 156.5774062 | 258.4101248 | Trim14 |
| ENSMUSG00000039981 | 53.27507895 | 77.24485371 | 62.64487874 | Zc3h12d |
| ENSMUSG00000040093 | 75.65061211 | 69.93790809 | 132.1415411 | Bmf |
| ENSMUSG00000040128 | 3368.050491 | 2671.210549 | 2388.336002 | Pnrc1 |
| ENSMUSG00000040152 | 91.63313579 | 40.71012561 | 85.15788203 | Thbs1 |
| ENSMUSG00000040435 | 430.4626379 | 241.1292055 | 201.6382034 | Ppp1r15a |
| ENSMUSG00000040584 | 118.2706753 | 124.2180756 | 233.939469 | Abcb1a |
| ENSMUSG00000040855 | 259.9823853 | 223.3837661 | 409.1493642 | Reps2 |
| ENSMUSG00000040891 | 1650.461946 | 1922.770548 | 1525.990093 | Foxa3 |
| ENSMUSG00000041134 | 18.11352684 | 31.31548123 | 39.15304921 | Cyyr1 |
| ENSMUSG00000041372 | 9.589514211 | 12.52619249 | 9.788262303 | B4galnt3 |
| ENSMUSG00000041695 | 101.22265 | 78.28870309 | 41.11070167 | Kcnj2 |
| ENSMUSG00000041702 | 59.66808842 | 58.45556497 | 97.88262303 | Btbd7 |
| ENSMUSG00000041920 | 586.0258684 | 355.9526367 | 312.2455675 | Slc16a6 |
| ENSMUSG00000041930 | 157.6942337 | 126.3057743 | 71.45431481 | Fam222a |
| ENSMUSG00000041945 | 3.196504737 | 5.219246872 | 10.76708853 | Mfsd9 |
| ENSMUSG00000042010 | 4018.006454 | 2149.285862 | 2550.821156 | Acacb |
| ENSMUSG00000042115 | 117.2051737 | 124.2180756 | 131.1627149 | Klhdc8a |
| ENSMUSG00000042246 | 77.78161526 | 33.40317998 | 54.81426889 | Tmc7 |
| ENSMUSG00000042333 | 30.89954579 | 27.14008374 | 41.11070167 | Tnfrsf14 |
| ENSMUSG00000042354 | 922.7243674 | 424.8466954 | 389.5728396 | Gnl3 |
| ENSMUSG00000042379 | 140.6462084 | 79.33255246 | 203.5958559 | Esm1 |
| ENSMUSG00000042444 | 255.720379 | 221.2960674 | 319.0973511 | Mindy2 |
| ENSMUSG00000042510 | 94.82964053 | 45.92937248 | 47.96248528 | AA986860 |
| ENSMUSG00000042607 | 37.29255526 | 24.00853561 | 42.0895279 | Asb4 |
| ENSMUSG00000042622 | 89.50213263 | 50.10476998 | 72.43314104 | Maff |
| ENSMUSG00000042680 | 87.37112948 | 114.8234312 | 243.7277313 | Garem1 |
| ENSMUSG00000042743 | 15.98252368 | 14.61389124 | 26.42830822 | Sgtb |
| ENSMUSG00000042745 | 726.6720769 | 889.3596671 | 654.834748 | Id1 |
| ENSMUSG00000043165 | 13.85152053 | 41.75397498 | 38.17422298 | Lor |
| ENSMUSG00000043421 | 138.5152053 | 55.32401685 | 75.36961973 | Hilpda |
| ENSMUSG00000043639 | 20.24453 | 46.97322185 | 49.92013774 | Rbm20 |
| ENSMUSG00000043681 | 543.4058053 | 474.9514654 | 382.721056 | Fam25c |
| ENSMUSG00000044042 | 108.6811611 | 85.59564871 | 203.5958559 | Fmn1 |
| ENSMUSG00000044186 | 17.04802526 | 10.43849374 | 17.61887214 | Nkx2-6 |
| ENSMUSG00000044339 | 61.79909158 | 89.77104621 | 79.28492465 | Alkbh2 |
| ENSMUSG00000044349 | 148.1047195 | 83.50794996 | 50.89896397 | Snhg11 |
| ENSMUSG00000044359 | 69.25760263 | 76.20100434 | 148.781587 | P2ry4 |
| ENSMUSG00000044676 | 609.4669032 | 360.1280342 | 270.1560396 | Zfp612 |
| ENSMUSG00000044749 | 1050.584557 | 874.7457758 | 2096.645785 | Abca6 |
| ENSMUSG00000044948 | 73.51960895 | 52.19246872 | 59.70840005 | Cfap43 |
| ENSMUSG00000045045 | 38.35805684 | 38.62242686 | 56.77192136 | Lrfn4 |
| ENSMUSG00000045294 | 8335.418852 | 7066.860265 | 6456.337815 | Insig1 |
| ENSMUSG00000045348 | 38.35805684 | 44.8855231 | 20.55535084 | Nyap1 |
| ENSMUSG00000045382 | 89.50213263 | 68.89405872 | 29.36478691 | Cxcr4 |
| ENSMUSG00000045411 | 798.0606826 | 403.9697079 | 303.4361314 | 2410002F23Rik |
| ENSMUSG00000045776 | 514.6372626 | 321.5056073 | 424.8105839 | Lrtm1 |
| ENSMUSG00000045875 | 28.76854263 | 45.92937248 | 72.43314104 | Adra1a |
| ENSMUSG00000046541 | 22.37553316 | 22.96468624 | 41.11070167 | Zfp526 |
| ENSMUSG00000046721 | 46.88206947 | 46.97322185 | 42.0895279 | Rpl14-ps1 |
| ENSMUSG00000046908 | 36.22705368 | 28.18393311 | 24.47065576 | Ltb4r1 |
| ENSMUSG00000047496 | 124.6636847 | 234.8661093 | 298.5420002 | Rnf152 |
| ENSMUSG00000047649 | 210.9693126 | 155.5335568 | 111.5861902 | Cd3eap |
| ENSMUSG00000047875 | 49.01307263 | 46.97322185 | 47.96248528 | Gpr157 |
| ENSMUSG00000048191 | 53.27507895 | 40.71012561 | 27.40713445 | Muc6 |
| ENSMUSG00000048644 | 40.48906 | 37.57857748 | 23.49182953 | Ctxn1 |
| ENSMUSG00000048856 | 8878.824658 | 12487.57007 | 11318.1677 | Slc25a47 |
| ENSMUSG00000049044 | 3411.736056 | 3040.733228 | 2890.473858 | Rapgef4 |
| ENSMUSG00000049313 | 152.3667258 | 109.6041843 | 170.3157641 | Sorl1 |
| ENSMUSG00000049580 | 1707.999031 | 1943.647535 | 1562.206663 | Tsku |
| ENSMUSG00000049791 | 215.231319 | 290.1901261 | 450.2600659 | Fzd4 |
| ENSMUSG00000049950 | 94.82964053 | 44.8855231 | 35.23774429 | Rpp38 |
| ENSMUSG00000050390 | 692.5760263 | 634.6604197 | 674.4112726 | C77080 |
| ENSMUSG00000050503 | 25.5720379 | 38.62242686 | 16.64004591 | Fbxl22 |
| ENSMUSG00000050663 | 15.98252368 | 13.57004187 | 15.66121968 | Trhde |
| ENSMUSG00000050737 | 119.3361768 | 55.32401685 | 31.32243937 | Ptges |
| ENSMUSG00000050914 | 273.8339058 | 58.45556497 | 46.98365905 | Ankrd37 |
| ENSMUSG00000051149 | 6.393009474 | 3.131548123 | 21.53417707 | Adnp |
| ENSMUSG00000051339 | 917.3968595 | 914.4120521 | 942.6096597 | 2900026A02Rik |
| ENSMUSG00000051452 | 92.69863737 | 80.37640184 | 127.2474099 | Gm11437 |
| ENSMUSG00000051674 | 295.1439374 | 270.356988 | 333.7797445 | Dcun1d4 |
| ENSMUSG00000051998 | 42.62006316 | 39.66627623 | 21.53417707 | Lax1 |
| ENSMUSG00000052085 | 197.1177921 | 161.796653 | 227.0876854 | Dock8 |
| ENSMUSG00000052595 | 821.5017174 | 661.8005034 | 1485.858218 | A1cf |
| ENSMUSG00000052656 | 1950.933391 | 1650.325861 | 1785.379044 | Rnf103 |
| ENSMUSG00000052684 | 547.6678116 | 268.2692892 | 399.3611019 | Jun |
| ENSMUSG00000052713 | 23.44103474 | 18.78928874 | 42.0895279 | Zfp608 |
| ENSMUSG00000052837 | 1233.850828 | 697.2913822 | 1027.767542 | Junb |
| ENSMUSG00000053560 | 725.6065753 | 569.9417585 | 550.1003414 | Ier2 |
| ENSMUSG00000053964 | 348.4190163 | 621.0903778 | 388.5940134 | Lgals4 |
| ENSMUSG00000053977 | 57.53708526 | 21.92083686 | 40.13187544 | Cd8a |
| ENSMUSG00000054008 | 753.3096163 | 842.3864452 | 2156.354185 | Ndst1 |
| ENSMUSG00000054150 | 33.03054895 | 29.22778249 | 32.3012656 | Syne3 |
| ENSMUSG00000054422 | 57173.74923 | 60050.56682 | 33819.42508 | Fabp1 |
| ENSMUSG00000054453 | 69.25760263 | 36.53472811 | 39.15304921 | Sytl5 |
| ENSMUSG00000054659 | 15.98252368 | 25.05238499 | 65.58135743 | Pm20d2 |
| ENSMUSG00000054932 | 72.45410737 | 72.02560684 | 80.26375088 | Afp |
| ENSMUSG00000055148 | 476.2792058 | 390.3996661 | 293.6478691 | Klf2 |
| ENSMUSG00000055254 | 409.1526063 | 418.5835992 | 554.9944726 | Ntrk2 |
| ENSMUSG00000055491 | 706.4275469 | 442.5921348 | 309.3090888 | Pprc1 |
| ENSMUSG00000055660 | 29.83404421 | 36.53472811 | 60.68722628 | Mettl4 |
| ENSMUSG00000055692 | 90.56763421 | 57.4117156 | 44.04718036 | Tmem191c |
| ENSMUSG00000055980 | 114.0086689 | 159.7089543 | 270.1560396 | Irs1 |
| ENSMUSG00000056054 | 100.1571484 | 98.1218412 | 95.92497057 | S100a8 |
| ENSMUSG00000056071 | 140.6462084 | 203.550628 | 123.332105 | S100a9 |
| ENSMUSG00000056091 | 3671.718441 | 3506.290049 | 4394.929774 | St3gal5 |
| ENSMUSG00000056148 | 23.44103474 | 25.05238499 | 50.89896397 | Rdh9 |
| ENSMUSG00000056313 | 1762.339612 | 990.6130564 | 874.0918236 | Tcim |
| ENSMUSG00000057342 | 564.7158369 | 644.0550641 | 614.7028726 | Sphk2 |
| ENSMUSG00000057604 | 25.5720379 | 28.18393311 | 27.40713445 | Lmcd1 |
| ENSMUSG00000057722 | 323.91248 | 199.3752305 | 166.4004591 | Lepr |
| ENSMUSG00000057969 | 49.01307263 | 42.79782435 | 37.19539675 | Sema3b |
| ENSMUSG00000058207 | 48372.70618 | 79528.79614 | 62218.1105 | Serpina3k |
| ENSMUSG00000058503 | 376.1220574 | 383.0927204 | 227.0876854 | Fam133b |
| ENSMUSG00000058793 | 1397.938072 | 1320.469459 | 1959.610113 | Cds2 |
| ENSMUSG00000058794 | 71.38860579 | 26.09623436 | 30.34361314 | Nfe2 |
| ENSMUSG00000058921 | 261.0478868 | 329.8564023 | 683.2207087 | Slc10a5 |
| ENSMUSG00000059149 | 147.0392179 | 80.37640184 | 237.854774 | Mfsd4a |
| ENSMUSG00000059824 | 24.50653632 | 16.70158999 | 12.72474099 | Dbp |
| ENSMUSG00000060429 | 201.3797984 | 180.5859418 | 380.7634036 | Sntb1 |
| ENSMUSG00000061175 | 495.4582342 | 405.0135573 | 478.6460266 | Fnip2 |
| ENSMUSG00000061292 | 523.1612753 | 399.7943104 | 986.6568401 | Cyp3a59 |
| ENSMUSG00000061436 | 228.0173379 | 186.849038 | 275.0501707 | Hipk2 |
| ENSMUSG00000061536 | 88.43663105 | 94.99029308 | 144.8662821 | Sec22c |
| ENSMUSG00000061825 | 298.3404421 | 160.7528037 | 130.1838886 | Ces2c |
| ENSMUSG00000062901 | 297.2749405 | 259.9184942 | 513.8837709 | Klhl24 |
| ENSMUSG00000063535 | 27.70304105 | 29.22778249 | 32.3012656 | Zfp773 |
| ENSMUSG00000063704 | 581.7638621 | 784.9747296 | 446.344761 | Mapk15 |
| ENSMUSG00000063929 | 208.8383095 | 167.0158999 | 174.231069 | Cyp4a32 |
| ENSMUSG00000065126 | 154.497729 | 158.6651049 | 77.32727219 | Snord104 |
| ENSMUSG00000065147 | 54.34058053 | 32.35933061 | 19.57652461 | Snora31 |
| ENSMUSG00000065952 | 51.14407579 | 55.32401685 | 76.34844596 | C330021F23Rik |
| ENSMUSG00000066456 | 70.32310421 | 34.44702936 | 38.17422298 | Hmgn3 |
| ENSMUSG00000066477 | 93.76413895 | 118.9988287 | 119.4168001 | Gm16551 |
| ENSMUSG00000066687 | 472.0171995 | 943.6398345 | 1137.39608 | Zbtb16 |
| ENSMUSG00000066944 | 10.65501579 | 10.43849374 | 24.47065576 | NA |
| ENSMUSG00000067149 | 437.921149 | 878.9211733 | 284.838433 | Jchain |
| ENSMUSG00000068463 | 46.88206947 | 15.65774062 | 12.72474099 | B630019A10Rik |
| ENSMUSG00000068742 | 312.1919626 | 260.9623436 | 334.7585707 | Cry2 |
| ENSMUSG00000068877 | 201.3797984 | 256.7869461 | 255.4736461 | Selenbp2 |
| ENSMUSG00000069456 | 2190.671246 | 1455.126028 | 1756.014257 | Rdh16 |
| ENSMUSG00000069804 | 42.62006316 | 55.32401685 | 34.25891806 | Gm10277 |
| ENSMUSG00000070576 | 98.02614527 | 132.5688706 | 122.3532788 | Mn1 |
| ENSMUSG00000070583 | 13.85152053 | 11.48234312 | 31.32243937 | Fv1 |
| ENSMUSG00000071076 | 3733.517533 | 2948.874483 | 1847.045097 | Jund |
| ENSMUSG00000071456 | 12.78601895 | 16.70158999 | 35.23774429 | 1110002L01Rik |
| ENSMUSG00000071547 | 90.56763421 | 104.3849374 | 70.47548858 | Nt5dc2 |
| ENSMUSG00000071637 | 239.7378553 | 123.1742262 | 171.2945903 | Cebpd |
| ENSMUSG00000071645 | 605.2048969 | 525.0562354 | 343.5680068 | Tut1 |
| ENSMUSG00000072294 | 24.50653632 | 18.78928874 | 60.68722628 | Klf12 |
| ENSMUSG00000072571 | 20.24453 | 29.22778249 | 29.36478691 | Tmem253 |
| ENSMUSG00000072664 | 273.8339058 | 440.504436 | 724.3314104 | Ugt3a1 |
| ENSMUSG00000072692 | 73.51960895 | 70.98175747 | 58.72957382 | Rpl37rt |
| ENSMUSG00000072849 | 570.0433447 | 342.3825948 | 378.8057511 | Serpina1e |
| ENSMUSG00000072999 | 294.0784358 | 417.5397498 | 310.287915 | Gm15401 |
| ENSMUSG00000073460 | 51.14407579 | 79.33255246 | 37.19539675 | Pnldc1 |
| ENSMUSG00000073835 | 85.24012632 | 69.93790809 | 16.64004591 | Mup-ps12 |
| ENSMUSG00000074024 | 118.2706753 | 117.9549793 | 98.86144926 | 4632427E13Rik |
| ENSMUSG00000074063 | 2843.823714 | 6567.900264 | 5870.020903 | Osgin1 |
| ENSMUSG00000074213 | 24.50653632 | 27.14008374 | 18.59769837 | Gm10642 |
| ENSMUSG00000074345 | 26.63753947 | 24.00853561 | 29.36478691 | Tnfaip8l3 |
| ENSMUSG00000074375 | 860.9252758 | 876.8334746 | 855.4941252 | Sult2a3 |
| ENSMUSG00000074876 | 71.38860579 | 41.75397498 | 54.81426889 | Spata5l1 |
| ENSMUSG00000075470 | 77.78161526 | 80.37640184 | 251.5583412 | Deaf1 |
| ENSMUSG00000075552 | 1023.947017 | 947.815232 | 894.6471745 | Cyp3a41b |
| ENSMUSG00000075590 | 1682.426993 | 1456.169877 | 1087.475942 | Nrbp2 |
| ENSMUSG00000076490 | 35.16155211 | 20.87698749 | 18.59769837 | Trbc1 |
| ENSMUSG00000076569 | 72.45410737 | 110.6480337 | 183.0405051 | Igkv5-39 |
| ENSMUSG00000076596 | 152.3667258 | 28.18393311 | 26.42830822 | Igkv3-10 |
| ENSMUSG00000076609 | 4135.211628 | 4467.675323 | 1384.06029 | Igkc |
| ENSMUSG00000076613 | 100.1571484 | 155.5335568 | 99.84027549 | Ighg2b |
| ENSMUSG00000076617 | 5037.691465 | 5276.658588 | 2480.345667 | Ighm |
| ENSMUSG00000076934 | 77.78161526 | 36.53472811 | 58.72957382 | Iglv1 |
| ENSMUSG00000077148 | 9.589514211 | 19.83313812 | 11.74591476 | Gm22935 |
| ENSMUSG00000078193 | 42.62006316 | 35.49087873 | 30.34361314 | Gm2000 |
| ENSMUSG00000078234 | 197.1177921 | 263.0500424 | 351.3986167 | Klhdc7a |
| ENSMUSG00000078650 | 6922.563759 | 10279.82864 | 10371.64274 | G6pc |
| ENSMUSG00000078651 | 55.40608211 | 57.4117156 | 43.06835413 | Aoc2 |
| ENSMUSG00000078672 | 337.7640005 | 374.7419254 | 237.854774 | Mup20 |
| ENSMUSG00000078688 | 49.01307263 | 38.62242686 | 39.15304921 | Mup2 |
| ENSMUSG00000078817 | 204.5763032 | 280.7954817 | 342.5891806 | Nlrp12 |
| ENSMUSG00000079017 | 145.9737163 | 124.2180756 | 88.09436072 | Ifi27l2a |
| ENSMUSG00000079036 | 575.3708526 | 288.1024274 | 180.1040264 | Alkbh1 |
| ENSMUSG00000079065 | 371.8600511 | 353.864938 | 194.7864198 | BC005561 |
| ENSMUSG00000079465 | 46.88206947 | 56.36786622 | 36.21657052 | Col4a3 |
| ENSMUSG00000079470 | 201.3797984 | 169.1035987 | 244.7065576 | Utp14b |
| ENSMUSG00000080059 | 37.29255526 | 13.57004187 | 19.57652461 | Rps19-ps3 |
| ENSMUSG00000081344 | 64.99559632 | 55.32401685 | 43.06835413 | Gm14303 |
| ENSMUSG00000082065 | 60.73359 | 39.66627623 | 5.872957382 | Mup-ps14 |
| ENSMUSG00000082173 | 41.55456158 | 31.31548123 | 8.809436072 | Mup-ps10 |
| ENSMUSG00000082586 | 15.98252368 | 7.306945621 | 17.61887214 | Sult2a-ps1 |
| ENSMUSG00000082658 | 85.24012632 | 129.4373224 | 49.92013774 | Fau-ps2 |
| ENSMUSG00000083327 | 36.22705368 | 34.44702936 | 66.56018366 | Vcp-rs |
| ENSMUSG00000083621 | 54.34058053 | 48.01707123 | 31.32243937 | Gm14586 |
| ENSMUSG00000083716 | 70.32310421 | 86.63949808 | 39.15304921 | Gm13436 |
| ENSMUSG00000083813 | 86.3056279 | 84.55179933 | 92.00966564 | Gm15502 |
| ENSMUSG00000083863 | 64.99559632 | 54.28016747 | 47.96248528 | Gm13341 |
| ENSMUSG00000083992 | 39.42355842 | 40.71012561 | 29.36478691 | Gm11478 |
| ENSMUSG00000084822 | 66.0610979 | 91.85874496 | 58.72957382 | Myadml2os |
| ENSMUSG00000084883 | 24.50653632 | 30.27163186 | 63.62370497 | Ccdc85c |
| ENSMUSG00000085001 | 156.6287321 | 70.98175747 | 70.47548858 | Rapgef4os2 |
| ENSMUSG00000085156 | 157.6942337 | 59.49941435 | 34.25891806 | Snhg15 |
| ENSMUSG00000085445 | 126.7946879 | 143.0073643 | 187.9346362 | Gm16348 |
| ENSMUSG00000085834 | 1120.907661 | 744.264604 | 450.2600659 | Gm15622 |
| ENSMUSG00000085995 | 444.3141584 | 772.4485371 | 450.2600659 | Gm2788 |
| ENSMUSG00000086140 | 11.72051737 | 22.96468624 | 25.44948199 | Hnf1aos2 |
| ENSMUSG00000086446 | 27.70304105 | 97.07799183 | 26.42830822 | Prkag2os1 |
| ENSMUSG00000086529 | 13.85152053 | 8.350794996 | 2.936478691 | Acss2os |
| ENSMUSG00000086786 | 2.131003158 | 3.131548123 | 15.66121968 | Gm15908 |
| ENSMUSG00000086844 | 73.51960895 | 42.79782435 | 46.98365905 | B230206H07Rik |
| ENSMUSG00000087382 | 414.4801142 | 369.5226786 | 445.3659348 | Ctcflos |
| ENSMUSG00000087445 | 61.79909158 | 54.28016747 | 39.15304921 | Gm14286 |
| ENSMUSG00000087595 | 41.55456158 | 62.63096247 | 36.21657052 | 1810012K08Rik |
| ENSMUSG00000087613 | 12.78601895 | 17.74543937 | 48.94131151 | Gm13855 |
| ENSMUSG00000087616 | 154.497729 | 34.44702936 | 26.42830822 | Gm14257 |
| ENSMUSG00000087658 | 36.22705368 | 28.18393311 | 23.49182953 | Hotairm1 |
| ENSMUSG00000089726 | 293.0129342 | 129.4373224 | 86.13670826 | Mir17hg |
| ENSMUSG00000089943 | 781.0126574 | 515.661591 | 1296.944755 | Ugt1a5 |
| ENSMUSG00000090021 | 15.98252368 | 39.66627623 | 48.94131151 | Gm6493 |
| ENSMUSG00000090145 | 154.497729 | 220.252218 | 375.8692724 | Ugt1a6b |
| ENSMUSG00000090175 | 125.7291863 | 209.8137243 | 409.1493642 | Ugt1a9 |
| ENSMUSG00000090264 | 57.53708526 | 80.37640184 | 52.85661643 | Eif4ebp3 |
| ENSMUSG00000090369 | 27.70304105 | 27.14008374 | 41.11070167 | 4933411K16Rik |
| ENSMUSG00000090555 | 1396.87257 | 624.2219259 | 491.3707676 | Gm8893 |
| ENSMUSG00000090610 | 35.16155211 | 28.18393311 | 22.5130033 | Gm3571 |
| ENSMUSG00000090698 | 247.1963663 | 60.54326372 | 76.34844596 | Apold1 |
| ENSMUSG00000091021 | 82.04362158 | 27.14008374 | 23.49182953 | Gm17300 |
| ENSMUSG00000091509 | 143.8427132 | 183.7174899 | 79.28492465 | Gm17066 |
| ENSMUSG00000092075 | 83.10912316 | 163.8843518 | 57.75074759 | Serpina4-ps1 |
| ENSMUSG00000094410 | 47.94757105 | 25.05238499 | 56.77192136 | Gm38394 |
| ENSMUSG00000095280 | 126.7946879 | 158.6651049 | 55.79309512 | Gm21738 |
| ENSMUSG00000095351 | 629.7114332 | 1051.15632 | 311.2667412 | Igkv3-2 |
| ENSMUSG00000096833 | 3.196504737 | 4.175397498 | 9.788262303 | Igkv4-55 |
| ENSMUSG00000096910 | 34.09605053 | 10.43849374 | 48.94131151 | Zfp955b |
| ENSMUSG00000096954 | 33.03054895 | 17.74543937 | 37.19539675 | Gdap10 |
| ENSMUSG00000097124 | 23.44103474 | 33.40317998 | 25.44948199 | A530020G20Rik |
| ENSMUSG00000097221 | 8.524012632 | 9.39464437 | 27.40713445 | 1810049J17Rik |
| ENSMUSG00000097312 | 114.0086689 | 161.796653 | 45.02600659 | Gm26870 |
| ENSMUSG00000097536 | 13.85152053 | 26.09623436 | 18.59769837 | 2610037D02Rik |
| ENSMUSG00000097615 | 69.25760263 | 65.76251059 | 70.47548858 | Gm2061 |
| ENSMUSG00000097660 | 60.73359 | 53.2363181 | 10.76708853 | Gm26762 |
| ENSMUSG00000097691 | 75.65061211 | 98.1218412 | 102.7767542 | 9030616G12Rik |
| ENSMUSG00000097743 | 155.5632305 | 98.1218412 | 88.09436072 | Gm16973 |
| ENSMUSG00000097908 | 52.20957737 | 45.92937248 | 57.75074759 | 4933404O12Rik |
| ENSMUSG00000097971 | 8345.008366 | 6440.550641 | 3776.311596 | Gm26917 |
| ENSMUSG00000097994 | 29.83404421 | 48.01707123 | 38.17422298 | Gm26982 |
| ENSMUSG00000098041 | 68.19210105 | 69.93790809 | 30.34361314 | Gm26981 |
| ENSMUSG00000098661 | 43.68556474 | 48.01707123 | 36.21657052 | Mir7052 |
| ENSMUSG00000098814 | 13.85152053 | 157.6212555 | 4.894131151 | Igkv19-93 |
| ENSMUSG00000098882 | 37.29255526 | 18.78928874 | 10.76708853 | Mir6392 |
| ENSMUSG00000099568 | 18.11352684 | 59.49941435 | 47.96248528 | Gm28513 |
| ENSMUSG00000099858 | 56.47158369 | 75.15715496 | 114.5226689 | Gm6652 |
| ENSMUSG00000100094 | 1017.554008 | 731.7384115 | 815.3622498 | 1810008I18Rik |
| ENSMUSG00000100468 | 6.393009474 | 6.263096247 | 9.788262303 | Tmem167-ps1 |
| ENSMUSG00000101939 | 52.20957737 | 56.36786622 | 46.00483282 | Gm28438 |
| ENSMUSG00000102275 | 56.47158369 | 54.28016747 | 18.59769837 | Gm37144 |
| ENSMUSG00000102577 | 137.4497037 | 252.6115486 | 199.680551 | Gm37969 |
| ENSMUSG00000102719 | 38.35805684 | 30.27163186 | 8.809436072 | Gm37760 |
| ENSMUSG00000102869 | 86.3056279 | 81.42025121 | 216.3205969 | 2900097C17Rik |
| ENSMUSG00000102882 | 26.63753947 | 38.62242686 | 15.66121968 | Gm2065 |
| ENSMUSG00000102918 | 18.11352684 | 14.61389124 | 27.40713445 | Pcdhgc3 |
| ENSMUSG00000103285 | 27.70304105 | 35.49087873 | 14.68239345 | Gm37274 |
| ENSMUSG00000103546 | 28.76854263 | 33.40317998 | 17.61887214 | Gm37666 |
| ENSMUSG00000104030 | 56.47158369 | 39.66627623 | 21.53417707 | 5330406M23Rik |
| ENSMUSG00000104388 | 46.88206947 | 34.44702936 | 12.72474099 | Gm37033 |
| ENSMUSG00000104399 | 77.78161526 | 41.75397498 | 42.0895279 | Gm37963 |
| ENSMUSG00000104445 | 38.35805684 | 40.71012561 | 25.44948199 | Rhbg |
| ENSMUSG00000104973 | 36.22705368 | 32.35933061 | 32.3012656 | A530041M06Rik |
| ENSMUSG00000105161 | 44.75106632 | 39.66627623 | 23.49182953 | Gm42595 |
| ENSMUSG00000105434 | 26.63753947 | 42.79782435 | 12.72474099 | Gm43359 |
| ENSMUSG00000105547 | 23.44103474 | 27.14008374 | 61.66605251 | Iglc3 |
| ENSMUSG00000105556 | 7.458511053 | 9.39464437 | 12.72474099 | Gm43080 |
| ENSMUSG00000105703 | 712.8205563 | 708.7737253 | 409.1493642 | Gm43305 |
| ENSMUSG00000105881 | 43.68556474 | 106.4726362 | 160.5275018 | 4932422M17Rik |
| ENSMUSG00000105906 | 182.20077 | 62.63096247 | 23.49182953 | Iglc1 |
| ENSMUSG00000106030 | 62.86459316 | 88.72719683 | 54.81426889 | Gm43611 |
| ENSMUSG00000106664 | 54.34058053 | 55.32401685 | 33.28009183 | Gm17936 |
| ENSMUSG00000106705 | 87.37112948 | 163.8843518 | 91.03083941 | Gm2602 |
| ENSMUSG00000106706 | 31.96504737 | 38.62242686 | 10.76708853 | C530043K16Rik |
| ENSMUSG00000106943 | 30.89954579 | 34.44702936 | 22.5130033 | Dancr |
| ENSMUSG00000107168 | 46.88206947 | 39.66627623 | 13.70356722 | Gm42507 |
| ENSMUSG00000107225 | 46.88206947 | 43.84167373 | 33.28009183 | Gm43637 |
| ENSMUSG00000107304 | 28.76854263 | 59.49941435 | 16.64004591 | Gm43775 |
| ENSMUSG00000107390 | 46.88206947 | 70.98175747 | 26.42830822 | Gm43323 |
| ENSMUSG00000107624 | 103.3536532 | 111.6918831 | 37.19539675 | Gm44005 |
| ENSMUSG00000108368 | 46.88206947 | 41.75397498 | 47.96248528 | Gm45053 |
| ENSMUSG00000108633 | 49.01307263 | 33.40317998 | 16.64004591 | Gm44694 |
| ENSMUSG00000108820 | 21.31003158 | 25.05238499 | 25.44948199 | Gm44620 |
| ENSMUSG00000108825 | 37.29255526 | 51.14861935 | 84.1790558 | Gm45838 |
| ENSMUSG00000109089 | 112.9431674 | 204.5944774 | 281.9019543 | 4833411C07Rik |
| ENSMUSG00000109115 | 4.262006316 | 4.175397498 | 8.809436072 | Gm44669 |
| ENSMUSG00000109157 | 25.5720379 | 30.27163186 | 18.59769837 | Gm44829 |
| ENSMUSG00000109262 | 14.91702211 | 19.83313812 | 12.72474099 | Gm44744 |
| ENSMUSG00000109291 | 17.04802526 | 38.62242686 | 25.44948199 | Gm2814 |
| ENSMUSG00000109536 | 109.7466626 | 90.81489558 | 68.51783612 | 9330162G02Rik |
| ENSMUSG00000109555 | 43.68556474 | 33.40317998 | 21.53417707 | Gm44891 |
| ENSMUSG00000109807 | 75.65061211 | 94.99029308 | 37.19539675 | Gm45244 |
| ENSMUSG00000109836 | 12.78601895 | 12.52619249 | 45.02600659 | Gm45884 |
| ENSMUSG00000109841 | 13.85152053 | 15.65774062 | 21.53417707 | E330011O21Rik |
| ENSMUSG00000110588 | 1747.42259 | 289.1462767 | 231.9818166 | Gm45774 |
| ENSMUSG00000110613 | 26.63753947 | 16.70158999 | 28.38596068 | Lncbate1 |
| ENSMUSG00000110702 | 45.8165679 | 30.27163186 | 25.44948199 | Gm45767 |
| ENSMUSG00000110755 | 79.91261842 | 28.18393311 | 20.55535084 | BC049987 |
| ENSMUSG00000111282 | 87.37112948 | 62.63096247 | 27.40713445 | Gm47528 |
| ENSMUSG00000111312 | 13.85152053 | 20.87698749 | 22.5130033 | Gm47205 |
| ENSMUSG00000111631 | 28.76854263 | 66.80635997 | 52.85661643 | Gm32017 |
| ENSMUSG00000111709 | 12.78601895 | 8.350794996 | 5.872957382 | Gm3776 |
| ENSMUSG00000111774 | 5.327507895 | 7.306945621 | 1.957652461 | AC166078.1 |

### Example 71: Phase 1b/2 Study of TGFRt15-TGFRs for Advanced Pancreatic Cancer

The study is a phase 1b/2, open label, multi-center, competitive enrollment and dose-escalation study of TGFRt15-TGFRs (HCW9218) in patients with advanced/metastatic pancreatic cancer. The study involves a Phase 1b dose escalation portion with up to 30 patients to determine the maximum tolerated dose (MTD) using a 3+3 dose escalation design and to designate a dose level for the Phase 2 expansion phase (RP2D).

The Phase 2 portion of the study will consist of an expansion cohort of up to 39 patients receiving TGFRt15-TGFRs monotherapy at the RP2D level. An additional independent Phase 2 cohort of patients receiving TGFRt15-TGFRs at the RP2D level in sequence with gemcitabine and nab-paclitaxel will also be considered.

### Outcome Measures

### Primary Outcome Measure Will Include:

1. Evaluate safety [Time Frame: 12 Months]—the safety profile (as outlined by incidence of adverse events (AEs) will be evaluated based on CTCAE v5) of TGFRt15-TGFRs monotherapy in subjects with advanced/metastatic pancreatic cancer who have progressed on or are intolerant of standard first-line therapy.
2. Determine the maximum tolerated dose (MTD) [Time Frame: 12 Months]—the maximum tolerated dose (MTD) will be determined and the recommended Phase 2 dose level (RP2D) for Phase 2 study of TGFRt15-TGFRs in TGFRt15-TGFRs-treated subjects will be designated.

### Secondary Outcome Measures Will Include:

1. Assess objective response rate [Time Frame: 12 Months]—objective response rate based on RECIST, progression-free survival, time to progression, duration of response, and overall survival will be assessed in TGFRt15-TGFRs-treated subjects.

### Eligibility Criteria

### Inclusion Criteria Will Include:

1. Subjects with histologically or cytologically confirmed unresectable, advanced/metastatic disease pancreatic cancer that has progressed on standard first-line (or second- or later line) systemic therapy (excepting progression within 6 months of end of adjuvant systemic chemotherapy); or subjects that can no longer be treated with first-line systemic therapy due to subject's intolerance.
2. For dose escalation phase (Phase 1b), subjects with distant metastatic disease or advanced disease and not a candidate for down staging to resection. For expansion phase (Phase 2), subjects with distant metastatic disease only.
3. Subjects with prior radiation are allowed if the index lesion(s) remains outside of the treatment field or has progressed since prior treatment. Radiation therapy must have been completed at least 4 weeks prior to the baseline scan.
4. Patient age should be 18 years or older.
5. Patient with a life expectancy of at least 12 weeks.
6. Laboratory tests performed within 14 days of treatment start:
   a. Absolute neutrophil count (AGC/ANC)≥1,500/μL (≥1.5×10⁹/L)
   b. Platelets≥100,000/μL (≥100×10⁹/L) [Subjects may be transfused not more than 1 unit of platelets within 2 weeks to meet this requirement]
   c. Hemoglobin≥9 g/dL (>90 g/L) [Subjects may be transfused not more than 2 units of pRBCs within 2 weeks to meet this requirement]
   d. Calculated glomerular filtration rate (GFR)*>40 mL/min OR serum creatinine≤1.5×ULN
   e. Total bilirubin≤2.0×ULN or ≤3.0×ULN for subjects with Gilbert's syndrome
   f. AST, ALT, ALP≤2.5×ULN or ≤5.0×ULN if liver metastasis present (*using the following Cockcroft & Gault equation to calculate the eGFR for this study.
eGFR in mL/min= [(140-age in years)×(weight in kg)×F]/(serum creatinine in mg/dL×72), where F=1 if male; and 0.85 for female.)
7. Subject with adequate pulmonary function with PFTs>50% FEV1 if symptomatic or prior known impairment.
8. Subject with negative serum pregnancy test within 14 days of treatment start if female and of childbearing potential (non-childbearing is defined as greater than one year postmenopausal or surgically sterilized).
9. Female subjects of childbearing potential must adhere to using a medically accepted method of birth control prior to screening and agree to continue its use for at least 28 days after the last dose of TGFRt15-TGFRs or be surgically sterilized (e.g., hysterectomy or tubal ligation) and males must agree to use a barrier method of birth control and agree to continue its use for at least 28 days after the last dose of TGFRt15-TGFRs.
10. Subjects should provide signed informed consent and HIPAA authorization and agree to comply with all protocol-specified procedures and follow-up evaluations.

### Exclusion Criteria Will Include:

Subjects with any of the following criteria are excluded from participation in the study (to be verified by Sponsor prior to subject enrollment):
1. History of clinically significant vascular disease, including any of the following within 6 months prior to start of study treatment: Ml or unstable angina, percutaneous coronary intervention, bypass grafting, ventricular arrhythmia requiring medication, stroke or transient ischemic attack, symptomatic peripheral arterial disease.
2. Marked baseline prolongation of QT/QTc interval (e.g., demonstration of a QTc interval greater than or equal to 470 milliseconds by Fridericia's correction).
3. Subjects with untreated CNS metastases are excluded. Subjects are eligible if CNS metastases are treated and subjects are neurologically stable for at least 2 weeks prior to enrollment. In addition, subjects must be either off corticosteroids, or on a stable or decreasing dose of s 10 mg daily prednisone (or equivalent).
4. Anti-cancer treatment including surgery, radiotherapy, chemotherapy, other immunotherapy, or investigational therapy within 14 days before treatment start
5. Other prior malignancy except for the following: adequately treated basal cell or squamous cell skin cancer, in situ cervical cancer, adequately treated Stage I or II cancer from which the subject is currently in complete remission, or any other cancer from which the subject has been disease-free for 3 years after surgical treatment.
6. Known hypersensitivity or history of allergic reactions attributed to compounds of similar chemical or biologic composition to the agents used in the study.
7. Prior therapy with TGF-β antagonist, IL-15 or analogs.
8. Concurrent herbal or unconventional therapy (e.g., St. John's Wort).
9. Known autoimmune disease requiring active treatment. Subjects with a condition requiring systemic treatment with either corticosteroids (>10 mg daily prednisone equivalent) or other immunosuppressive medications within 14 days of enrollment. Inhaled or topical steroids, and adrenal replacement steroid doses≤10 mg daily prednisone equivalent, are permitted in the absence of active autoimmune disease.
10. Active systemic infection requiring parenteral antibiotic therapy. All prior infections must have resolved following optimal therapy.
11. Prior organ allograft or allogeneic transplantation.
12. Known HIV-positive or AIDS.
13. Women who are pregnant or nursing.
14. Any ongoing toxicity from prior anti-cancer treatment that, in the judgment of the Investigator, may interfere with study treatment. All toxicities attributed to prior anticancer therapy other than peripheral neuropathy, alopecia, and fatigue must resolve to grade 1 (NCI CTCAE v5.0) or baseline before administration of the study treatment
15. Psychiatric illness/social situations that would limit compliance with study requirements.
16. Other illness or a medical issue that in the opinion of the Investigator would exclude the subject from participating in this study.

### Initially Enrolled Subjects

| | | | | | | | Prior lines | Prior | HCW9218 | HCW9218 |
|---|---|---|---|---|---|---|---|---|---|---|
| | Age | | | | | Sites of | of systemic | radiation | dose | doses |
| Patient | (yrs) | Gender | Race | Ethnicity | Disease | metastasis | therapy | surgey | level | received |
| [symbol] | 55 | Male | white | Non- | Metastatic | Liver | 1) FOLFIRNOX, | | 0.25 mg/kg | 1 |
| | | | | hispanic | pancreatic | | 2) Gemcitabine, | | | |
| | | | | | adenocarcinoma | | abraxane | | | |
| [symbol] | 63 | Male | White | Non- | Metastatic | Peritoneum | 1) Abraxane, cisplatin, | Cholecystectomy | 0.25 mg/kg | 1 |
| | | | | hispanic | pancreatic | | gemcitabine, 2) CM24 | | | |
| | | | | | adenocarcinoma | | | | | |
| [symbol] | 78 | Female | White | Non- | Metastatic | Lung | 1) [symbol] , cisplatin, | Total body | 0.25 mg/ng | 1 |
| | | | | hispanic | pancreatic | | gemcitabine, | irradiation (TBI) | | |
| | | | | | adenocarcinoma | | 2) [symbol] , 3) [symbol] | | | |
| | | | | | | | liposome, leucovorin, | | | |
| | | | | | | | [symbol] -FU, 4) [symbol] , | | | |
| | | | | | | | cisplatin, gemcitabine | | | |
| [symbol] | 57 | Male | White | Non- | Metastatic | Lung | 1) FOLFIRNOX, | TBI, Whipple | 0.25 mg/kg | 2 |
| | | | | hispanic | pancreatic | | 2) FOLFIRNOX (w/o | resection of | | |
| | | | | | adenocarcinoma | | [symbol] ), | pancreas | | |
| | | | | | | | 3) Gemcitabine, | | | |
| | | | | | | | 4) Gemcitabine, | | | |
| | | | | | | | 5) Gemcitabine, | | | |
| | | | | | | | capecitabine, | | | |
| | | | | | | | 6) investigational | | | |
| | | | | | | | Product | | | |
| [symbol] | 6 [symbol] | Male | White | Non- | Metastatic | Liver | 1) FOLFERINOX, | | 0.25 mg/kg | 1 |
| | | | | hispanic | pancreatic | | 2) Gemcitabine, | | | |
| | | | | | ductal | | abraxane, 3) [symbol] -FU, | | | |
| | | | | | adenocarcinoma | | [symbol] | | | |

[symbol]

indicates data missing or illegible when filed

Demographics, disease status, and study treatment of patients with pancreatic cancer receiving subcutaneous TGFRt15-TGFRs (HCW9218) every 4 weeks.

1. A method of treating unresectable advanced/metastatic pancreatic cancer in a subject, the method comprising administering to the subject a therapeutically effective amount of a multi-chain chimeric polypeptide, wherein the multi-chain chimeric polypeptide comprises:
(a) a first chimeric polypeptide comprising:
(i) a first target-binding domain;
(ii) a soluble tissue factor domain comprising a sequence that is at least 90% identical to SEQ ID NO: 5; and
(iii) a first domain of a pair of affinity domains comprising a sequence that is at least 90% identical to SEQ ID NO: 15;
(b) a second chimeric polypeptide comprising:
(i) a second domain of a pair of affinity domains comprising a sequence that is at least 90% identical to SEQ ID NO: 29; and
(ii) a second target-binding domain,
wherein:
the first chimeric polypeptide and the second chimeric polypeptide associate through the binding of the first domain and the second domain of the pair of affinity domains; and
the first target-binding domain binds specifically to a ligand of TGF-β receptor II (TGF-βRII) and comprises a sequence that is at 90% identical to SEQ ID NO: 69, and the second target-binding domain binds specifically to a ligand of TGF-βRII and comprises a sequence that is at least 90% identical to SEQ ID NO: 69.

2. (canceled)

3. A method of increasing progression-free survival or progression-free survival rate in a subject or population of subjects having unresectable advanced/metastatic pancreatic cancer, the method comprising administering to the subject(s) a therapeutically effective amount of a multi-chain chimeric polypeptide, wherein the multi-chain chimeric polypeptide comprises:
(a) a first chimeric polypeptide comprising:
(i) a first target-binding domain;
(ii) a soluble tissue factor domain comprising a sequence that is at least 90% identical to SEQ ID NO: 5; and
(iii) a first domain of a pair of affinity domains comprising a sequence that is at least 90% identical to SEQ ID NO: 15;
(b) a second chimeric polypeptide comprising:
(i) a second domain of a pair of affinity domains comprising a sequence that is at least 90% identical to SEQ ID NO: 29; and
(ii) a second target-binding domain,
wherein:
the first chimeric polypeptide and the second chimeric polypeptide associate through the binding of the first domain and the second domain of the pair of affinity domains; and
the first target-binding domain binds specifically to a ligand of TGF-β receptor II (TGF-βRII) and comprises a sequence that is at 90% identical to SEQ ID NO: 69, and the second target-binding domain binds specifically to a ligand of TGF-βRII and comprises a sequence that is at least 90% identical to SEQ ID NO: 69.

4-7. (canceled)

8. The method of claim 1, wherein the subject(s) has/have received previous treatment with standard first-line systemic therapy for pancreatic cancer, and the subject's/subjects' pancreatic cancer had progressed on and/or was intolerant to the previous treatment.

9. (canceled)

10. The method of claim 8, wherein the standard first-line systemic therapy comprises one or more of: FOLFIRINOX, modified FOLFINIROX, gemcitabine, albumin-bound paclitaxel, cisplatin, erlotinib, capecitabine, docetaxel, fluoropyrimidine, and oxaliplatin.

11. The method of claim 10, wherein the first-line systemic therapy comprises one of:
(i) FOLFIRINOX;
(ii) modified FOLFIRINOX;
(iii) gemcitabine and albumin-bound paclitaxel;
(iv) gemcitabine and erlotinib;
(v) gemcitabine;
(vi) gemcitabine and capecitabine;
(vii) gemcitabine, docetaxel, and capecitabine; or
(viii) fluoropyrimidine and oxaliplatin.

12. The method of claim 10, wherein the subject(s) has/have previously been identified as having a BRCA1, BRCA2, or PALB2 mutation, and the first-line systemic therapy comprises one of:
(i) FOLFIRINOX;
(ii) modified FOLFIRINOX; or
(iii) gemcitabine and cisplatin.

13. The method of claim 1, wherein the subject(s) has/have received previous treatment with second- or later-line systemic therapy for pancreatic cancer, and the subject's/subjects' pancreatic cancer had progressed on and/or was intolerant to the previous treatment.

14. The method of claim 13, wherein the second- or later-line systemic therapy comprises one or more of: a different first-line systemic therapy, 5-fluorouracil, leucovorin, liposomal irinotecan, irinotecan, FOLFIRINOX, modified FOLFIRINOX, oxaliplatin, FOLFOX, capecitabine, gemcitabine, albumin-bound paclitaxel, cisplatin, erlotinib, pembrolizumab, larotrectinib, or entrectinib.

15. The method of claim 14, wherein the second- or later-line systemic therapy is a different first-line systemic therapy.

16. The method of claim 14, wherein the second- or later-line systemic therapy comprises one of:
(i) 5-fluorouracil, leucovorin, and liposomal irinotecan;
(ii) 5-fluorouracil, leucovorin, and irinotecan (FOLFIRI);
(iii) FOLFIRINOX or modified FOLFIRINOX;
(iv) oxaliplatin, 5-fluorouracil, and leucovorin (OFF);
(v) FOLFOX;
(vi) capecitabine and oxaliplatin;
(vii) capecitabine; or
(viii) continuous infusion 5-fluorouracil.

17. The method of claim 14, wherein the subject(s) was/were previously treated with fluoropyrimidine-based therapy and the second- or later-line systemic therapy comprises one of:
(i) gemcitabine;
(ii) gemcitabine and albumin-bound paclitaxel; or
(iii) gemcitabine with erlotinib.

18. The method of claim 14, wherein the subject(s) was/were previously treated with fluoropyrimidine-based therapy and was/were previously identified as having a BRCA1, BRCA2, or PALB2 mutation, and the second- or later-line systemic therapy comprises gemcitabine and cisplatin.

19. The method of claim 14, wherein the subject(s) was/were previously treated with fluoropyrimidine-based therapy and has/have not received prior treatment with irinotecan, and the second- or later-line systemic therapy comprises 5-fluorouracil, leucovorin, and liposomal irinotecan.

20. The method of claim 14, wherein the subject(s) was/were previously identified as having an MSI-H or dMMR tumor, and the second- or later-line systemic therapy comprises pembrolizumab.

21. The method of claim 14, wherein the subject(s) was/were previously identified as having a NTRK gene fusion, and the second- or later-line systemic therapy comprises larotrectinib or entrectinib.

22. The method of claim 1, wherein the subject(s) has/have distant metastatic disease.

23-28. (canceled)

29. The method of claim 1, wherein the subject(s) has/have:
an absolute neutrophil count of greater than or equal to 1,500/microliter;
a platelet count of greater than or equal to 100,000/microliter;
a hemoglobin level of greater than or equal to 9 g/dL;
a glomerular filtration rate (GFR) of greater than 40 mL/min or serum creatinine level of less than or equal to 1.5×Upper Limit of Normal (ULN);
a total bilirubin level of less than or equal to 2.0×ULN or less than or equal to 3.0×ULN for subjects having Gilbert's syndrome; or
aspartate aminotransferase (AST), alanine aminotransferase (ALT), and alkaline phosphatase (ALP) levels of less than or equal to 2.5×ULN or less than or equal to 5.0×ULN if liver metastasis is present.

30-39. (canceled)

40. The method of claim 1, wherein the subject(s) has/have not received surgery, radiotherapy, chemotherapy, other immunotherapy, or investigational therapy within 14 days prior to the administering step.

41. The method of claim 1, wherein the subject(s) does/do not have any other prior malignancy except for adequately-treated basal cell or squamous cell skin cancer, in situ cervical cancer, adequately-treated stage I or II cancer from which the subject(s) is/are currently in complete remission, or any other cancer from which the subject(s) has/have been disease-free for 3 years after surgical treatment.

42. (canceled)

43. The method of claim 1, wherein the subject(s) has/have not received prior treatment with a TGF-beta antagonist or IL-15 or analog thereof.

44-77. (canceled)

78. The method of claim 1, wherein the first chimeric polypeptide comprises a sequence that is at least 90% identical to SEQ ID NO: 70.

79-90. (canceled)

91. The method of claim 1, wherein the second chimeric polypeptide comprises a sequence that is at least 90% identical to SEQ ID NO: 74.

92-94. (canceled)

95. The method of claim 1, wherein the multi-chain chimeric polypeptide is subcutaneously administered to the subject(s).

96. The method of claim 1, wherein the subject(s) is/are administered a single dose of the multi-chain chimeric polypeptide.

97. The method of claim 96, wherein the single dose is 0.1 mg of the multi-chain chimeric polypeptide per kg of the subject's body weight (mg/kg), 0.25 mg/kg, 0.5 mg/kg, 0.8 mg/kg, or 1.2 mg/kg.

98-101. (canceled)

102. The method of claim 1, wherein the subject(s) is/are administered two or more doses of the multi-chain chimeric polypeptide over a treatment period.

103. The method of claim 102, wherein at least one of the two or more doses is 0.1 mg of the multi-chain chimeric polypeptide per kg of the subject's body weight (mg/kg), 0.25 mg/kg, 0.5 mg/kg, 0.8 mg/kg, or 1.2 mg/kg.

104-107. (canceled)

108. The method of claim 102, wherein the treatment period is about 4 weeks.


SEQUENCE LISTING (77 sequences)

SEQ ID NO: 1 - protein, 219 aa, Homo sapiens
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 2 - DNA, 657 bp, Homo sapiens
  source 1..657: mol_type = genomic DNA
agcggcacaaccaacacagtcgctgcctataacctcacttggaagagcaccaacttcaaaaccatcctcgaatgggaacccaaacccgttaaccaagtttacaccgtgcagatcagcaccaagtccggcgactggaagtccaaatgtttctataccaccgacaccgagtgcgatctcaccgatgagatcgtgaaagatgtgaaacagacctacctcgcccgggtgtttagctaccccgccggcaatgtggagagcactggttccgctggcgagcctttatacgagaacagccccgaatttaccccttacctcgagaccaatttaggacagcccaccatccaaagctttgagcaagttggcacaaaggtgaatgtgacagtggaggacgagcggactttagtgcggcggaacaacacctttctcagcctccgggatgtgttcggcaaagatttaatctacacactgtattactggaagtcctcttcctccggcaagaagacagctaaaaccaacacaaacgagtttttaatcgacgtggataaaggcgaaaactactgtttcagcgtgcaagctgtgatcccctcccggaccgtgaataggaaaagcaccgatagccccgttgagtgcatgggccaagaaaagggcgagttccgggag

SEQ ID NO: 3 - protein, 219 aa, Homo sapiens
SGTTNTVAAYNLTWKSTNFATALEWEPKPVNQVYTVQISTKSGDWKSKCFYTTDTECALTDEIVKDVKQTYLARVFSYPAGNVESTGSAGEPLYENSPEFTPYLETNLGQPTIQSFEQVGTKVNVTVEDERTLVARNNTALSLRDVFGKDLIYTLYYWKSSSSGKKTAKTNTNEFLIDVDKGENYCFSVQAVIPSRTVNRKSTDSPVECMGQEKGEFRE

SEQ ID NO: 4 - protein, 219 aa, Homo sapiens
SGTTNTVAAYNLTWKSTNFATALEWEPKPVNQVYTVQISTKSGDAKSKCFYTTDTECALTDEIVKDVKQTYLARVFSYPAGNVESTGSAGEPLAENSPEFTPYLETNLGQPTIQSFEQVGTKVNVTVEDERTLVARNNTALSLRDVFGKDLIYTLYYWKSSSSGKKTAKTNTNEFLIDVDKGENYCFSVQAVIPSRTVNRKSTDSPVECMGQEKGEFRE

SEQ ID NO: 5 - protein, 223 aa, Mus musculus
AGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGE

SEQ ID NO: 6 - protein, 224 aa, Rattus rattus
AGTPPGKAFNLTWISTDFKTILEWQPKPTNYTYTVQISDRSRNWKYKCTGTTDTECDLTDEIVKDVNWTYEARVLSVPWRNSTHGKETLFGTHGEEPPFTNARKFLPYRDTKIGQPVIQKYEQGGTKLKVTVKDSFTLVRKNGTFLTLRQVFGNDLGYILTYRKDSSTGRKTNTTHTNEFLIDVEKGVSYCFFAQAVIFSRKTNHKSPESITKCTEQWKSVLGE

SEQ ID NO: 7 - protein, 15 aa, Synthetic construct
  REGION 1..15: note = Linker
GGGGSGGGGSGGGGS

SEQ ID NO: 8 - protein, 45 aa, Synthetic construct
  REGION 1..45: note = Linker
GGCGGTGGAGGATCCGGAGGAGGTGGCTCCGGCGGCGGAGGATCT

SEQ ID NO: 9 - protein, 8 aa, Synthetic construct
  REGION 1..8: note = Linker
GGGSGGGS

SEQ ID NO: 10 - protein, 133 aa, Homo sapiens [sequence omitted]

SEQ ID NO: 11 - protein, 133 aa, Homo sapiens
APMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 12 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 13 - protein, 79 aa, Homo sapiens
EGAVLPRSAKELRCQCIKTYSKPFHPKFIKELRVIESGPHCANTEIIVKLSDGRELCLDPKENWVQRVVEKFLKRAENS

SEQ ID NO: 14 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 15 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 16 - protein, 132 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 17 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 18 - protein, 352 aa, Homo sapiens
RDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 19 - protein, 248 aa, Homo sapiens
EGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 20 - protein, 209 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 21 - protein, 360 aa, Homo sapiens
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 22 - protein, 361 aa, Homo sapiens
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTDFPYVSAAMPCFVIIIILCVPCCKKKTSAAEGPELVSLQVLDQHPVGTGDHRDAAQLGFQPLMSATGSTGSTEGA

SEQ ID NO: 23 - protein, 190 aa, Homo sapiens
WVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPG

SEQ ID NO: 24 - protein, 191 aa, Homo sapiens
GRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMS

SEQ ID NO: 25 - protein, 188 aa, Homo sapiens
DAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAPG

SEQ ID NO: 26 - protein, 233 aa, Homo sapiens
HSLCFNFTIKSLSRPGQPWCEAQVFLNKNLFLQYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPVNASDIHWSSSSLPDRWIILGAFILLVLMGIVLICVWWQNGEWQAGLWPLRTS

SEQ ID NO: 27 - protein, 193 aa, Homo sapiens
GLADPHSLCYDITVIPKFRPGPRWCAVQGQVDEKTFLHYDCGSKTVTPVSPLGKKLNVTTAWKAQNPVLREVVDILTEQLLDIQLENYIPKEPLTLQARMSCEQKAEGHGSGSWQLSFDGQIFLLFDSENRMWTTVHPGARKMKEKWENDKDMTMSFHYISMGDCTGWLEDFLMGMDSTLEPSAGAPPTMSSG

SEQ ID NO: 28 - protein, 193 aa, Homo sapiens
RRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSG

SEQ ID NO: 29 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 30 - DNA, 195 bp, Homo sapiens
  source 1..195: mol_type = genomic DNA
attacatgcccccctcccatgagcgtggagcacgccgacatctgggtgaagagctatagcctctacagccgggagaggtatatctgtaacagcggcttcaagaggaaggccggcaccagcagcctcaccgagtgcgtgctgaataaggctaccaacgtggctcactggacaacaccctctttaaagtgcatccgg

SEQ ID NO: 31 - DNA, 342 bp, Homo sapiens
  source 1..342: mol_type = genomic DNA
aactgggtgaacgtcatcagcgatttaaagaagatcgaagatttaattcagtccatgcatatcgacgccactttatacacagaatccgacgtgcacccctcttgtaaggtgaccgccatgaaatgttttttactggagctgcaagttatctctttagagagcggagacgctagcatccacgacaccgtggagaatttaatcattttagccaataactctttatccagcaacggcaacgtgacagagtccggctgcaaggagtgcgaagagctggaggagaagaacatcaaggagtttctgcaatcctttgtgcacattgtccagatgttcatcaatacctcc

SEQ ID NO: 32 - skipped

SEQ ID NO: 33 - DNA, 54 bp, Synthetic construct
  source 1..54: mol_type = other DNA
atgaaatgggtgacctttatttctttactgttcctctttagcagcgcctactcc

SEQ ID NO: 34 - DNA, 54 bp, Synthetic construct
  source 1..54: mol_type = other DNA
atgaagtgggtcacatttatctctttactgttcctcttctccagcgcctacagc

SEQ ID NO: 35 - DNA, 54 bp, Synthetic construct; identical to SEQ ID NO: 33
  source 1..54: mol_type = other DNA

SEQ ID NO: 36 - protein, 16 aa, Synthetic construct
  REGION 1..16: note = Signal sequence
MKCLLYLAFLFLGVNC

SEQ ID NO: 37 - protein, 58 aa, Synthetic construct
  REGION 1..58: note = Signal sequence
MGQIVTMFEALPHIIDEVINIVIIVLIIITSIKAVYNFATCGILALVSFLFLAGRSCG

SEQ ID NO: 38 - protein, 97 aa, Synthetic construct
  REGION 1..97: note = Signal sequence
MPNHQSGSPTGSSDLLLSGKKQRPHLALRRKRRREMRKINRKVRRMNLAPIKEKTAWQHLQALISEAEEVLKTSQTPQNSLTLFLALLSVLGPPVTG

SEQ ID NO: 39 - protein, 30 aa, Synthetic construct
  REGION 1..30: note = Signal sequence
MDSKGSSQKGSRLLLLLVVSNLLLCQGVVS

SEQ ID NO: 40 - protein, 15 aa, Synthetic construct
  REGION 1..15: note = AviTag
GLNDIFEAQKIEWHE

SEQ ID NO: 41 - protein, 26 aa, Synthetic construct
  REGION 1..26: note = calmodulin tag
KRRWKKNFIAVSAANRFKKISSSGAL

SEQ ID NO: 42 - protein, 6 aa, Synthetic construct
  REGION 1..6: note = polyglutamate tag
EEEEEE

SEQ ID NO: 43 - protein, 13 aa, Synthetic construct
  REGION 1..13: note = E-tag
GAPVPYPDPLEPR

SEQ ID NO: 44 - protein, 8 aa, Synthetic construct
  REGION 1..8: note = FLAG-tag
DYKDDDDK

SEQ ID NO: 45 - protein, 9 aa, Synthetic construct
  REGION 1..9: note = HA-tag
YPYDVPDYA

SEQ ID NO: 46 - protein, 5 aa, Synthetic construct
  REGION 1..5: note = His-tag
HHHHH

SEQ ID NO: 47 - protein, 6 aa, Synthetic construct
  REGION 1..6: note = His-tag
HHHHHH

SEQ ID NO: 48 - protein, 7 aa, Synthetic construct
  REGION 1..7: note = His-tag
HHHHHHH

SEQ ID NO: 49 - protein, 8 aa, Synthetic construct
  REGION 1..8: note = His-tag
HHHHHHHH

SEQ ID NO: 50 - protein, 9 aa, Synthetic construct
  REGION 1..9: note = His-tag
HHHHHHHHH

SEQ ID NO: 51 - protein, 10 aa, Synthetic construct
  REGION 1..10: note = His-tag
HHHHHHHHHH

SEQ ID NO: 52 - protein, 10 aa, Synthetic construct
  REGION 1..10: note = myc-tag
EQKLISEEDL

SEQ ID NO: 53 - protein, 18 aa, Synthetic construct
  REGION 1..18: note = NE-tag
TKENPRSNQEESYDDNES

SEQ ID NO: 54 - protein, 15 aa, Synthetic construct
  REGION 1..15: note = S-tag
KETAAAKFERQHMDS

SEQ ID NO: 55 - protein, 38 aa, Synthetic construct
  REGION 1..38: note = SBP-tag
MDEKTTGWRGGHVVEGLAGELEQLRARLEHHPQGQREP

SEQ ID NO: 56 - protein, 13 aa, Synthetic construct
  REGION 1..13: note = Softag 1
SLAELLNAGLGGS

SEQ ID NO: 57 - protein, 8 aa, Synthetic construct
  REGION 1..8: note = Softag 3
TQDPSRVG

SEQ ID NO: 58 - protein, 12 aa, Synthetic construct
  REGION 1..12: note = Spot-tag
PDRVRAVSHWSS

SEQ ID NO: 59 - protein, 8 aa, Synthetic construct
  REGION 1..8: note = Strep-tag
WSHPQFEK

SEQ ID NO: 60 - protein, 6 aa, Synthetic construct
  REGION 1..6: note = TC tag
CCPGCC

SEQ ID NO: 61 - protein, 10 aa, Synthetic construct
  REGION 1..10: note = Ty tag
EVHTNQDPLD

SEQ ID NO: 62 - protein, 14 aa, Synthetic construct
  REGION 1..14: note = V5 tag
GKPIPNPLLGLDST

SEQ ID NO: 63 - protein, 11 aa, Synthetic construct
  REGION 1..11: note = VSV-tag
YTDIEMNRLGK

SEQ ID NO: 64 - protein, 8 aa, Synthetic construct
  REGION 1..8: note = Xpress tag
DLYDDDDK

SEQ ID NO: 65 - protein, 10 aa, Synthetic construct
  REGION 1..10: note = myc tag
EQKLISEEDL

SEQ ID NO: 66 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 67 - DNA, 408 bp, Homo sapiens
  source 1..408: mol_type = genomic DNA
attcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgcacaatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgac

SEQ ID NO: 68 - DNA, 861 bp, Synthetic construct
  source 1..861: mol_type = other DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcacgatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgcacaatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgac

SEQ ID NO: 69 - protein, 287 aa, Synthetic construct
  REGION 1..287: note = soluble human TGFBRII
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 70 - protein, 620 aa, Synthetic construct
  REGION 1..620: note = First Chimeric Polypeptide
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 71 - DNA, 1860 bp, Synthetic construct
  source 1..1860: mol_type = other DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacagcggcacaaccaacacagtcgctgcctataacctcacttggaagagcaccaacttcaaaaccatcctcgaatgggaacccaaacccgttaaccaagtttacaccgtgcagatcagcaccaagtccggcgactggaagtccaaatgtttctataccaccgacaccgagtgcgatctcaccgatgagatcgtgaaagatgtgaaacagacctacctcgcccgggtgtttagctaccccgccggcaatgtggagagcactggttccgctggcgagcctttatacgagaacagccccgaatttaccccttacctcgagaccaatttaggacagcccaccatccaaagctttgagcaagttggcacaaaggtgaatgtgacagtggaggacgagcggactttagtgcggcggaacaacacctttctcagcctccgggatgtgttcggcaaagatttaatctacacactgtattactggaagtcctcttcctccggcaagaagacagctaaaaccaacacaaacgagtttttaatcgacgtggataaaggcgaaaactactgtttcagcgtgcaagctgtgatcccctcccggaccgtgaataggaaaagcaccgatagccccgttgagtgcatgggccaagaaaagggcgagttccgggagaactgggtgaacgtcatcagcgatttaaagaagatcgaagatttaattcagtccatgcatatcgacgccactttatacacagaatccgacgtgcacccctcttgtaaggtgaccgccatgaaatgttttttactggagctgcaagttatctctttagagagcggagacgctagcatccacgacaccgtggagaatttaatcattttagccaataactctttatccagcaacggcaacgtgacagagtccggctgcaaggagtgcgaagagctggaggagaagaacatcaaggagtttctgcaatcctttgtgcacattgtccagatgttcatcaatacctcc

SEQ ID NO: 72 - protein, 638 aa, Synthetic construct
  REGION 1..638: note = First Chimeric Polypeptide
MKWVTFISLLFLFSSAYSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 73 - DNA, 1914 bp, Synthetic construct
  source 1..1914: mol_type = other DNA
atgaagtgggtgaccttcatcagcctgctgttcctgttctccagcgcctactccatccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacagcggcacaaccaacacagtcgctgcctataacctcacttggaagagcaccaacttcaaaaccatcctcgaatgggaacccaaacccgttaaccaagtttacaccgtgcagatcagcaccaagtccggcgactggaagtccaaatgtttctataccaccgacaccgagtgcgatctcaccgatgagatcgtgaaagatgtgaaacagacctacctcgcccgggtgtttagctaccccgccggcaatgtggagagcactggttccgctggcgagcctttatacgagaacagccccgaatttaccccttacctcgagaccaatttaggacagcccaccatccaaagctttgagcaagttggcacaaaggtgaatgtgacagtggaggacgagcggactttagtgcggcggaacaacacctttctcagcctccgggatgtgttcggcaaagatttaatctacacactgtattactggaagtcctcttcctccggcaagaagacagctaaaaccaacacaaacgagtttttaatcgacgtggataaaggcgaaaactactgtttcagcgtgcaagctgtgatcccctcccggaccgtgaataggaaaagcaccgatagccccgttgagtgcatgggccaagaaaagggcgagttccgggagaactgggtgaacgtcatcagcgatttaaagaagatcgaagatttaattcagtccatgcatatcgacgccactttatacacagaatccgacgtgcacccctcttgtaaggtgaccgccatgaaatgttttttactggagctgcaagttatctctttagagagcggagacgctagcatccacgacaccgtggagaatttaatcattttagccaataactctttatccagcaacggcaacgtgacagagtccggctgcaaggagtgcgaagagctggaggagaagaacatcaaggagtttctgcaatcctttgtgcacattgtccagatgttcatcaatacctcc

SEQ ID NO: 74 - protein, 352 aa, Synthetic construct
  REGION 1..352: note = Second Chimeric Polypeptide
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIR

SEQ ID NO: 75 - DNA, 1056 bp, Synthetic construct
  source 1..1056: mol_type = other DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacattacatgcccccctcccatgagcgtggagcacgccgacatctgggtgaagagctatagcctctacagccgggagaggtatatctgtaacagcggcttcaagaggaaggccggcaccagcagcctcaccgagtgcgtgctgaataaggctaccaacgtggctcactggacaacaccctctttaaagtgcatccgg

SEQ ID NO: 76 - protein, 370 aa, Synthetic construct
  REGION 1..370: note = Second Chimeric Polypeptide
MKWVTFISLLFLFSSAYSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIR

SEQ ID NO: 77 - DNA, 1110 bp, Synthetic construct
  source 1..1110: mol_type = other DNA
atgaagtgggtgaccttcatcagcctgctgttcctgttctccagcgcctactccatccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacattacatgcccccctcccatgagcgtggagcacgccgacatctgggtgaagagctatagcctctacagccgggagaggtatatctgtaacagcggcttcaagaggaaggccggcaccagcagcctcaccgagtgcgtgctgaataaggctaccaacgtggctcactggacaacaccctctttaaagtgcatccgg